(19) United States
(12) Patent Application Publication
(10) Pub. No.: US 2007/0292849 A1
(43) Pub. Date: Dec. 20, 2007

(54) Methods for Identifying Risk of Low Bmd and Treatments Thereof

(76) Inventors: Steven Mah, New York, NY (US); Carolyn R Hoyal-Wrightson, San Diego, CA (US); Stefan Kammerer, San Diego, CA (US); Matthew R Nelson, San Diego, CA (US); Andreas Braun, San Diego, CA (US); Rikard H. Reneland, Knivsta (SE)

Correspondence Address: GRANT ANDERSON LLP;C/O PORTFOLIOIP, PO BOX 52050, MINNEAPOLIS, MN, 55402, US

(21) Appl. No.: 10/578,392

(22) PCT Filed: Dec. 29, 2005
(86) PCT No.: PCT/US05/47411

§ 371 Date: Jun. 27, 2007

Related U.S. Application Data
(60) Provisional application No. 60/640,966, filed on Dec. 31, 2004.

(51) Int. Cl. C12Q 1/68

(52) U.S. Cl. 435/6

(57) ABSTRACT

Provided herein are methods for identifying a risk of low BMD in a subject, reagents and kits for carrying out the methods, methods for identifying candidate therapeutics for treating low BMD-related disorders, such as osteoporosis, and therapeutic and preventative methods applicable to osteoporosis. These embodiments are based upon an analysis of polymorphic variations in nucleotide sequences within the human genome.

### RELATED APPLICATIONS

This patent application claims the benefit of U.S. Provisional Patent Application No. 60/640,966 filed on 31 Dec. 2004, entitled “Methods For Identifying Risk Of Low BMD And Treatments Thereof,” naming Steven Mah et al. as inventors, and designated by attorney docket no. SEQ-4095-PV. This patent application is related to U.S. Provisional Patent Application Nos. 60/640,967 and 60/640,796, each filed on 31 Dec. 2004, entitled “Methods For Identifying Risk Of Low BMD And Treatments Thereof,” naming Steven Mah et al. as inventors, and designated by attorney docket nos. SEQ-4094-PV and SEQ-4096-PV, respectively. The content and subject matter of each of these patent applications is hereby incorporated by reference in its entirety, including all text and drawings, in jurisdictions providing for such incorporation.

### FIELD OF THE INVENTION

The invention relates to genetic methods for identifying susceptibility to low bone mineral density (BMD) and/or bone damage generally associated with human diseases, and in particular to osteoporosis, and treatments that specifically target the disease.

### BACKGROUND

Osteoporosis is a common disease characterized by low bone mineral density (BMD), deterioration of bone micro-architecture and increased risk of bone damage, such as fracture. Common types of osteoporosis include postmenopausal and senile osteoporosis, which generally occur later in life, e.g., 70+ years.

Osteoporosis is a major public health problem which affects quality of life and increases costs to health care providers. It is estimated that 44 million Americans and 100 million people worldwide are at risk for osteoporosis. In the United States today, 10 million individuals are estimated to already have the disease and almost 34 million more, or 55% of the people 50 years of age and older, have low bone mass, which puts them at increased risk of developing osteoporosis and related fractures. Of the 10 million Americans estimated to have osteoporosis, eight million are women and 2 million are men. These numbers are growing as the elderly population increases. It is estimated that by the middle of the next century the number of osteoporosis sufferers will double in the West, but may increase six-fold in Asia and South America. The estimated national direct expenditures (e.g., hospitals and nursing homes) for osteoporotic and associated fractures was $17 billion in 2001 ($47 million each day)—and the cost is rising (See National Osteoporosis Foundation; http://www.nof.org/osteoporosis/stats.htm).

Fracture is the most serious endpoint of osteoporosis, particularly fracture of the hip which affects up to 1.7 million people worldwide each year. One in two women and one in four men over age 50 will have an osteoporosis-related fracture in their lifetime. It is estimated that by the year 2050, the number of hip fractures worldwide will increase to over 6 million, as life expectancy and age of the population increase (See Spangler et al. “The Genetic Component of Osteoporosis Mini-review”; http:www.csa.com.osteointro.html).

Peak bone mass is mainly genetically determined, though dietary factors and physical activity can have positive effects. Peak bone mass occurs when skeletal growth ceases, after which time bone loss starts. In contrast to the positive balance that occurs during growth, in osteoporosis, the resorbed cavity is not completely refilled by bone and BMD decreases. Based on studies of family histories, twin studies, and racial factors, some attribute 50-60% of total bone variation (e.g., Bone Mineral Density to genetic effects and suggest there may be a predisposition for osteoporosis.

Osteoporosis can be considered a complex genetic trait with variants of several genes underlying the genetic determination of the variability of the phenotype. Low BMD is an important risk factor for fractures, the clinically most relevant feature of osteoporosis. Segregation analysis in families has shown that BMD is under polygenic control. In addition, biochemical markers of bone turnover have shown to have strong genetic components. Several candidate genes have been analyzed in relation to BMD, but the most widely studied gene in this respect, the vitamin D receptor (VDR) gene, explains only a small part of the genetic effect on BMD. Numerous studies, focusing on the BsmI allele of the vitamin D receptor gene have concluded that absence of the restriction site correlates with low bone mineral density.

### SUMMARY

It has been discovered that certain polymorphic variations in human genomic DNA are associated with the occurrence of low bone mineral density (BMD) and/or bone damage generally associated with human diseases, and in particular to osteoporosis. In particular, polymorphic variants in loci containing CETP, PROL4, GRID2, PDE4D or GPX3/TNIP1/TNIP1 regions in human genomic DNA have been associated with risk of low BMD.

Thus, featured herein are methods for identifying a subject at risk of low bone mineral density (BMD) and/or bone fracture, which indicates bone damage and related conditions such as osteoporosis in a subject. The methods comprise detecting the presence or absence of one or more of the polymorphic variations described herein in a human nucleic acid sample. In an embodiment, two or more polymorphic variations are detected and in some embodiments, 3 or more, or 4, 5, 6, 7, 8, 9, 10, 11, 12, 13, 14, 15, 16, 17, 18, 19 or 20 or more polymorphic variants are detected.

Also featured are nucleic acids that include one or more polymorphic variations associated with occurrence of low BMD, as well as polypeptides encoded by these nucleic acids. In addition, provided are methods for identifying candidate therapeutic molecules for osteoporosis and other low BMD-related disorders, as well as methods for treating osteoporosis in a subject by identifying a subject at risk of low BMD and treating the subject with a suitable prophylactic, treatment or therapeutic molecule.

Also provided are compositions comprising a cell from a subject suffering from low BMD or at risk of low BMD, and a CETP, PROL4, GRID2, PDE4D or GPX3/TNIP1/TNIP1 nucleic acid, with a RNAi, siRNA, antisense DNA or RNA, or ribozyme nucleic acid designed from a CETP, PROL4, GRID2, PDE4D or GPX3/TNIP1 nucleotide sequence, or a nucleic acid that hybridizes to such a nucleotide sequence under stringent conditions. In an embodiment, the RNAi, siRNA, antisense DNA or RNA, or ribozyme nucleic acid is designed from a CETP, PROL4, GRID2, PDE4D or GPX3/TNIP1 nucleotide sequence that includes one or more low BMD associated polymorphic variations, and in some instances, specifically interacts with such a nucleotide sequence. Further, provided are arrays of nucleic acids bound to a solid surface, in which one or more nucleic acid molecules of the array have a CETP, PROL4, GRID2, PDE4D or GPX3/TNIP1 nucleotide sequence, or a fragment or substantially identical nucleic acid thereof, or a complementary nucleic acid of the foregoing. Featured also are compositions comprising a cell from a subject having low BMD or at risk of low BMD and/or a CETP, PROL4, GRID2, PDE4D or GPX3/TNIP1 polypeptide, with an antibody that specifically binds to the polypeptide. In an embodiment, the antibody specifically binds to an epitope in the polypeptide that includes a non-synonymous amino acid modification associated with low BMD (e.g., results in an amino acid substitution in the encoded polypeptide associated with low BMD). In an embodiment, the antibody specifically binds to an epitope comprising an arginine corresponding to position 120 in a PROL4 polypeptide (SEQ ID NO: 12).

### BRIEF DESCRIPTION OF THE DRAWINGS

FIGS. 1-4 show the position of each SNP in the chromosome on the x-axis, while the y-axis provides the negative logarithm of the p-value comparing the estimated allele frequency in the cases to that of the control group. Also shown in the figures are exons and introns of the genes in approximate chromosomal positions. More specifically, FIG. 1 shows proximal SNPs in a CETP region in genomic DNA. FIG. 2 shows proximal SNPs in a PROW region in genomic DNA. FIG. 3 shows proximal SNPs in a GRID2 region in genomic DNA. FIG. 4 shows proximal SNPs in a PDE4D region in genomic DNA. FIG. 5 shows proximal SNPs in a GPX3/TNIP1 region in genomic DNA.

### DETAILED DESCRIPTION

It has been discovered that polymorphic variants described in a CETP, PROL4, GRID2, PDE4D and GPX3/TNIP1 loci in human genomic DNA are associated with occurrence of low BMD in subjects. Thus, detecting genetic determinants in and around this locus associated with an increased risk of low BMD occurrence can lead to early identification of a risk of low BMD, or its associated disorders such as osteoporosis, and early application of preventative and treatment measures. Associating the polymorphic variants with low BMD also has provided new targets for diagnosing low BMD, for prognosing osteoporosis, and methods for screening molecules useful in osteoporosis treatments and osteoporosis preventatives.

Cholestery ester transfer protein (CETP) transfers cholesteryl esters between lipoproteins. The transfer of insoluble cholesteryl esters among lipoprotein particles (HDL to LDL) by CETP is a step in normal cholesterol homeostasis. The lipoprotein phenotype of CETP deficiency, which is characterized by increased levels of HDL and decreased levels of low density lipoprotein (LDL), appears to have antiatherogenic potential. Bone mineral density is reduced by atherogenic diets (increased LDL/HDL ratios), possibly as a result of a shift in the balance of bone marrow stromal cells away from osteoblasts and towards adipocytes, which could result in reduced bone synthesis during remodeling. Lipoproteins may directly regulate bone density via the LRP5, the low-density lipoprotein receptor related protein 5. LRP5 was cloned from an osteoblast cDNA library (Dong, et al.: Biochem Biophys Res Commun. 1998 Oct. 29; 251(3):784-90) and mouse knockouts show a reduced BMD phenotype (Kato et. al.: J Cell Biol. 2002 Apr. 15; 157(2):303-14). A G171V mutation in LRP5 resulted in increased numbers of osteoblasts, increased AP activity, and increased trabecular number and thickness in mice (Babij et al.: J Bone Miner Res. 2003 June; 18(6): 960-74). The human G171V mutation was found to have the same phenotype, presumably by increasing WNT signaling in osteoblasts (Mao et. al.: Mol Cell. 2001 April; 7(4):801-9). Overall, there is evidence that lipoprotein profiles play a role in osteoblast differentiation. Provided is a method for treating osteoporosis or low bone mineral density by modulating a CETP function in a subject suffering from osteoporosis or low bone mineral density. CETP inhibitors and methods of making them are described in U.S. Pat. No. 6,586,613 (Substituted tetrahydronaphthaline and analogous compounds); U.S. Pat. No. 6,562,976 (4-phenyltetrahydrochinoline utilized as an inhibitor of the cholesterol ester transfer protein); U.S. Pat. No. 6,387,929 (4-heteroaryl-tetrahydroquinolines and their use as inhibitors of the cholesterin-ester transfer protein); U.S. Pat. No. 6,291,477 (Tetrahydroquinolines, processes for their preparation, pharmaceutical compositions containing them, and their use to prevent or treat hyperlipoproteinaemia); U.S. Pat. No. 6,218,431 (Substituted biphenyls); U.S. Pat. No. 6,207,671 (Cycloalkano-pyridines); U.S. Pat. No. 6,127,383 (2-aryl-substituted pyridines); U.S. Pat. No. 6,121,330 (5-Hydroxyalkyl substituted phenyls and their use in medicaments for the treatment of arteriosclerosis and hyperlipoproteinaemia); U.S. Pat. No. 6,069,148 (Cycloalkano-pyridines); U.S. Pat. No. 6,063,788 (Bicyclic-fused pyridines); U.S. Pat. No. 5,932,587 (Heterocyclic-fused pyridines); U.S. Pat. No. 5,925,645 (2-aryl-substituted pyridines); U.S. Pat. No. 6,753,346 (CETP activity inhibitor); U.S. Pat. No. 6,706,881 (Methods for preparing CETP inhibitors); U.S. Pat. No. 6,689,897 (Intermediates of CETP inhibitors); U.S. Pat. No. 6,600,045 (Methods for preparing CETP inhibitors); U.S. Pat. No. 6,573,383 (Preparation of anhydrous CETP inhibitor); U.S. Pat. No. 6,555,113 (Modulation of cholesteryl ester transfer protein (CETP) activity); U.S. Pat. No. 6,426,365 (CETP activity inhibitors); U.S. Pat. No. 6,410,022 (Modulation of cholesteryl ester transfer protein (CETP) activity); U.S. Pat. No. 6,410,020 (Monoclonal antibody reactive to human CETP and assay method for human CETP); U.S. Pat. No. 6,140,474 (Monoclonal antibody reactive with human-origin CETP and method of quantifying human-origin CETP); U.S. Pat. No. 5,948,435 (Methods of regulating CETP genes, enzymes and other compound, and pharmaceutical composition therefor); U.S. Pat. No. 5,519,001 (CETP inhibitor polypeptide antibodies against the synthetic polypeptide and prophylactic and therapeutic anti-atherosclerosis treatments); and U.S. Pat. No. 5,512,548 (CETP inhibitor polypeptide, antibodies against the synthetic polypeptide and prophylactic and therapeutic anti-atherosclerosis treatments).

PROL4, also known as Lacrimal proline rich protein (LPRP) is a member of the proline-rich secreted protein family and contains a conserved acidic N-terminal region. It has 45.5% amino acid homology to a salivary Parotid acidic protein (PRH1). PRH1 and related proline-rich (salivary) proteins act as potent inhibitors of hydroxyapatite crystal growth and bind calcium with a strength that suggests that they are important in maintaining the concentration of ionic calcium in saliva. The N-terminal region of salivary proline-rich proteins, which is also the region of strongest homology to PROL4, mediates this binding. It is expected that PROL4 plays a role in maintaining calcium levels during bone remodeling, and that a loss of function of PROL4 facilitates bone loss. Provided is a method of treating low BMD or osteoporosis by increasing expression of PROL4 or otherwise increasing levels of active PROL4 in a patient suffering from low BMD or osteoporosis.

Human glutamate receptor delta-2 (GRID2) is a member of the family of ionotropic glutamate receptors that are excitatory neurotransmitter receptors in mammalian brain. A point mutation in mouse GRID2, associated with the phenotype named “lurcher” in the heterozygous state, leads to ataxia resulting from selective, cell-autonomous apoptosis of cerebellar Purkinje cells during postnatal development. Mice homozygous for this mutation die shortly after birth from massive loss of mid- and hindbrain neurons during late embryogenesis. Glutamate receptors have been detected in osteoblasts and osteoclasts and may regulate bone resorption. Provided is a method of treating low bone mineral density or osteoporosis by modulating GRID2 function in a patient suffering from low BMD or osteoporosis.

PDE4D encodes cyclic AMP-dependent phosphodiesterase 4D. Phosphodiesterases are a superfamily of enzymes involved in degradation of cyclic adenosine monophosphate (cAMP) and cyclic guanosine monophosphate (cGMP) (Manganiello V C, et al.: Arch Biochem Biophys 1995, 322(1):1-13; and Beavo J A: Physiol Rev 1995, 75(4):725-748). cAMP and cGMP are important second messengers participating in the response of various cells to hormones. In osteoblasts, cAMP produced in response to parathyroid hormone or prostaglandins regulates osteoblastic differentiation (Farndale R W, et al. Biochem J 1988, 252(1):263-268; Kumegawa M, et al. Calcif Tissue Int 1984, 36(1):72-76; Ishizuya T, et al. J Clin Invest 1997, 99(12):2961-2970; and Partridge N C, et al. J Cell Biochem 1994, 55(3):321-327), which leads to increases in cancellous bone volume as indicated by experiments in animal models (Jee W S, et al. Bone 1987, 8(3):171-178; High W B: et al. Bone 1987, 8(6):363-373; Reeve J: et al. J Bone Miner Res 1996, 11(4):440-445; and Finkelstein J S, et al. N Engl J Med 1994, 331(24): 1618-1623). Intracellular levels of cAMP are regulated by G protein-coupled adenylyl cyclase (Casperson G F, Bourne H R: Annu Rev Pharmacol Toxicol 1987, 27:371-384), and degradation is mediated by the phosphodiesterases. The phosphodiesterase superfamily consists of seven families, PDE1-7, distinguished by substrate specificity, chromatographic behavior during purification, and affinity for biochemical activators and inhibitors. Of these, the PDE4 family is specific for cAMP and is selectively inhibited by rolipram. Four PDE4 genes, 4A, 4B, 4C, and 4D, have been cloned from rat and humans, all of which are predicted to have multiple protein products due to alternate spicing of RNAs. PDE4 inhibitors have been shown to increase bone formation in normal mice (Kinoshita T, et al. Bone 2000, 27(6):811-817) and to ameliorate loss of bone mass in animal models of osteopenia (Miyamoto K, et al. Biochem Pharmacol 1997, 54(5):613-617; and Waki Y, et al. Jpn J Pharmacol 1999, 79(4):477-483). PDE4A and PDE4D are expressed in two common mouse osteoblastic cell lines, ST2 and MC3T3-E1, that represent different stages in the osteoblast differentiation pathway (Wakabayashi S, et al. J Bone Miner Res 2002, 17(2):249-256). PDE4 inhibition with rolipram increased BMP2-induced alkaline phosphatase activity, a marker of early osteoblast differentiation in ST2 cells. Furthermore, rolipram increased the expression of alkalic phosphatase, osteopontin, collagen type I and osteocalcin in the same osteoblast precursor cells (Wakabayashi S, et al. J Bone Miner Res 2002, 17(2):249-256).

Provided herein is a method for treating osteoporosis or low bone mineral density by modulating a PDE4 function in a human suffering from osteoporosis or low bone mineral density. Modulation of PDE4D by PDE4D inhibitors may increase bone formation and treat an osteoporotic condition. PDE4D inhibitors and methods of making them are described in the following U.S. Pat. Nos. 6,218,400 (Treatment method using a cGMP-Specific PDE inhibitor), 5,891,896 (Tri-substituted phenyl derivatives useful as PDE IV inhibitors), 5,849,770 (Tri-substituted phenyl derivatives useful as PDE IV inhibitors), 5,712,298 (Fluoroalkoxy-substituted benzamides and their use as cyclic nucleotide phosphodiesterase inhibitors), 5,491,147 (Tri-substituted phenyl derivatives and their use in pharmaceutical compositions and methods of treatment). Also provided is a method of treating osteoporosis by decreasing expression of PDE4D or otherwise decreasing levels of active PDE4D in a subject suffering from low BMD. Provided also is a method of targeting pertinent information or administering preventative or therapeutic treatments to a subject based on a subject's PDE4D genotype.

SNP rs869975 is contained within the GPX3 gene, and the TNIP3 gene may not be ruled out due to linkage disequilibrium; therefore the GPX3/TNIP3 region is considered associated with low BMD. The GPX3 gene encodes the Plasma glutathione peroxidase 3 precursor. Glutathione peroxidase catalyzes the reduction of hydrogen peroxide, organic hydroperoxide, and lipid peroxides by reduced glutathione and functions in the protection of cells against oxidative damage. This enzyme, found mainly in the cytosol of mammalian cells, is unusual in its content of a selenocysteine residue in its active site that is encoded by a TGA opal codon. Selenium deficiency causes bone loss and might contribute to lower BMD. Osteoblasts produce glutathione peroxidase, possibly as a defense against hydrogen peroxide produced by osteoclasts during bone remodeling and thus may contribute to lower BMD. Provided is a method for treating osteoporosis or low bone mineral density by modulating a GPX function in a human suffering from osteoporosis or low bone mineral density.

In the TNIP1/NAF1/ABIN-1 pathway, NAF1 was identified by a yeast two-hybrid screen as an interacting protein to the HIV protein, Nef. Subsequently, it was found to be an A20-binding protein that is critical for the A20-mediated negative feedback regulation of NF-kappa B activation in response to tumor necrosis factor (TNF). As TNF is a critical effector of the pathogenesis of rheumatoid arthritis (RA), Gallagher et. al. (FEBS Lett. 2003 Sep. 11; 551(1-3):8-12), tested TNF-alpha-modulated gene expression in cultured primary human synoviocytes in vitro. Genes upregulated included TNIP1 and implicate TNIP1 as a potential modulator of TNF-alpha bioactivity in RA. The differentiation and functions of osteoclasts are stimulated and regulated by osteoblast/stromal cell derived factors, such as receptor activator of NFKB ligand (RANKL). Provided is a method for treating osteoporosis or low bone mineral density by modulating a TINIP function in a human suffering from osteoporosis or low bone mineral density.

Low BMD and Sample Selection

The present invention is applicable to any disease in which low BMD and/or bone fracture is a factor, and is therefore particularly concerned with diseases such as osteoporosis. Low BMD is defined by the World Health Organization as 2.5 standard deviations below the age-matched mean of bone mineral density for a given population. Bone damage may be defined as any form of structural damage such as fractures or chips of the bone, and degradation or deterioration of the bone other than normal wear and tear resulting from low bone mineral density or another cause. Such low BMD and/or bone damage is associated with osteoporosis.

Osteoporosis, or porous bone, is a disease characterized by low bone mass and structural deterioration of bone tissue, leading to bone fragility and an increased susceptibility to fractures, especially of the hip, spine and wrist. In general, there are two types of osteoporosis: primary and secondary. Approximately 90% of all osteoporosis cases is idiopathic “primary osteoporosis”. Such primary osteoporosis includes postmenopausal osteoporosis, age-associated osteoporosis (affecting a majority of individuals over the age of 70 to 80), and idiopathic osteoporosis affecting middle-aged and younger men and women. “Secondary osteoporosis” is the result of an identifiable disease process or agent.

For some osteoporotic individuals, the loss of bone tissue is sufficiently great so as to cause mechanical failure of the bone structure. Bone fractures often occur, for example, in the hip and spine of women suffering from postmenopausal osteoporosis. Kyphosis (abnormally increased curvature of the thoracic spine) may also result.

The mechanism of bone loss in osteoporotics is believed to involve an imbalance in the process of “bone remodeling”. Bone remodeling occurs throughout life, renewing the skeleton and maintaining the strength of bone. This remodeling involves the erosion and filling of discrete sites on the surface of bones, by an organized group of cells called “basic multicellular units” or “BMUs”. BMUs primarily consist of “osteoclasts”, “osteoblasts”, and their cellular precursors. In the remodeling cycle, bone is resorbed at the site of an “activated” BMU by an osteoclast, forming a resorption cavity. This cavity is then filled with bone by an osteoblast.

Normally, in adults, the remodeling cycle results in a small deficit in bone, due to incomplete filling of the resorption cavity. Thus, even in healthy adults, age-related bone loss occurs. However, in osteoporotics, there is an increase in the number of BMUs that are activated. This increased activation accelerates bone remodeling, resulting in abnormally high bone loss.

Preferred methods for the treatment of osteoporosis include an initial diagnostic step to determine the presence of the disorder. Initial diagnostic steps include determination of bone mass and rate of bone remodeling. The rate of bone remodeling can be determined by the measurement of biochemical markers. See, for example, Hui et al., “The Contribution of Bone Loss to Postmenopausal Osteoporosis” Osteoporosis Int. 30 (1990). Diagnosis of those at risk of developing osteoporosis also allows more effective preventive measures. Part of diagnosis includes specialized tests called bone density tests that measure bone density in various sites of the body. Such methods include the measurement of the radiodensity of skeletal radiographs, quantitative computerized tomography, single energy photon absorptiometry, and dual-energy photon absorptiometry. Diagnostic techniques among those useful herein are described in W. A. Peck et al., Physician's Resource Manual on Osteoporosis (1987), published by the National Osteoporosis Foundation. A bone density test can detect the presence of low BMD before a fracture occurs, predict your chances of fracturing in the future, determine rate of bone loss, and monitor response to treatment.

Based in part upon selection criteria set forth above, individuals having low BMD can be selected for genetic studies. Also, individuals having a family history of low BMD or diagnosed with osteoporosis often are selected for genetic studies. Other selection criteria can include: a tissue or fluid sample derived from an individual characterized as Caucasian; sample derived from an individual of Caucasian paternal and maternal descent, case samples derived from individuals diagnosed with osteoporosis; control samples derived from individuals with normal or high BMD levels and no family history of osteoporosis; and sufficient genomic DNA for all allelotyping and genotyping reactions performed during the study. Phenotype information may included pre- or post-menopausal, familial predisposition, country or origin of mother and father, diagnosis with osteoporosis (date of primary diagnosis, age of individual as of primary diagnosis, osteoporosis-related fracture), biochemical measurements of markers of bone resorption (bone-specific alkaline phosphatase, urinary C-telopeptide of type I collagen, serum osteocalcin), current medication status (thyroid medication, hormone replacement therapy, steroid usage, bisphosphonates and cytotoxic agents for rheumatic diseases). Samples that meet the inclusion criteria and do not meet the exclusion criteria may be added to appropriate pools based on gender and disease status.

Polymorphic Variants Associated with Low BMD

A genetic analysis provided herein linked low BMD with polymorphic variant nucleic acid sequences in the human genome. As used herein, the term “polymorphic site” refers to a region in a nucleic acid at which two or more alternative nucleotide sequences are observed in a significant number of nucleic acid samples from a population of individuals. A polymorphic site may be a nucleotide sequence of two or more nucleotides, an inserted nucleotide or nucleotide sequence, a deleted nucleotide or nucleotide sequence, or a microsatellite, for example. A polymorphic site that is two or more nucleotides in length may be 3, 4, 5, 6, 7, 8, 9, 10, 11, 12, 13, 14, 15 or more, 20 or more, 30 or more, 50 or more, 75 or more, 100 or more, 500 or more, or about 1000 nucleotides in length, where all or some of the nucleotide sequences differ within the region. A polymorphic site is often one nucleotide in length, which is referred to herein as a “single nucleotide polymorphism” or a “SNP.”

Where there are two, three, or four alternative nucleotide sequences at a polymorphic site, each nucleotide sequence is referred to as a “polymorphic variant” or “nucleic acid variant.” Where two polymorphic variants exist, for example, the polymorphic variant represented in a minority of samples from a population is sometimes referred to as a “minor allele” and the polymorphic variant that is more prevalently represented is sometimes referred to as a “major allele.” Many organisms possess a copy of each chromosome (e.g., humans), and those individuals who possess two major alleles or two minor alleles are often referred to as being “homozygous” with respect to the polymorphism, and those individuals who possess one major allele and one minor allele are normally referred to as being “heterozygous” with respect to the polymorphism. Individuals who are homozygous with respect to one allele are sometimes predisposed to a different phenotype as compared to individuals who are heterozygous or homozygous with respect to another allele.

In genetic analysis that associate polymorphic variants with low BMD, samples from individuals having low BMD and individuals not having low BMD often are allelotyped and/or genotyped. The term “allelotype” as used herein refers to a process for determining the allele frequency for a polymorphic variant in pooled DNA samples from cases and controls. By pooling DNA from each group, an allele frequency for each SNP in each group is calculated. These allele frequencies are then compared to one another. The term “genotyped” as used herein refers to a process for determining a genotype of one or more individuals, where a “genotype” is a representation of one or more polymorphic variants in a population.

A genotype or polymorphic variant may be expressed in terms of a “haplotype,” which as used herein refers to two or more polymorphic variants occurring within genomic DNA in a group of individuals within a population. For example, two SNPs may exist within a gene where each SNP position includes a cytosine variation and an adenine variation. Certain individuals in a population may carry one allele (heterozygous) or two alleles (homozygous) having the gene with a cytosine at each SNP position. As the two cytosines corresponding to each SNP in the gene travel together on one or both alleles in these individuals, the individuals can be characterized as having a cytosine/cytosine haplotype with respect to the two SNPs in the gene.

As used herein, the term “phenotype” refers to a trait which can be compared between individuals, such as presence or absence of a condition, a visually observable difference in appearance between individuals, metabolic variations, physiological variations, variations in the function of biological molecules, and the like. An example of a phenotype is occurrence of low BMD or clinically diagnosed osteoporosis.

Researchers sometimes report a polymorphic variant in a database without determining whether the variant is represented in a significant fraction of a population. Because a subset of these reported polymorphic variants are not represented in a statistically significant portion of the population, some of them are sequencing errors and/or not biologically relevant. Thus, it is often not known whether a reported polymorphic variant is statistically significant or biologically relevant until the presence of the variant is detected in a population of individuals and the frequency of the variant is determined. Methods for detecting a polymorphic variant in a population are described herein, specifically in Example 2. A polymorphic variant is statistically significant and often biologically relevant if it is represented in 5% or more of a population, sometimes 10% or more, 15% or more, or 20% or more of a population, and often 25% or more, 30% or more, 35% or more, 40% or more, 45% or more, or 50% or more of a population.

A polymorphic variant may be detected on either or both strands of a double-stranded nucleic acid. Also, a polymorphic variant may be located within an intron or exon of a gene or within a portion of a regulatory region such as a promoter, a 5′ untranslated region (UTR), a 3′ UTR, and in DNA (e.g., genomic DNA (gDNA) and complementary DNA (cDNA)), RNA (e.g., mRNA, tRNA, and rRNA), or a polypeptide. Polymorphic variations may or may not result in detectable differences in gene expression, polypeptide structure, or polypeptide function.

It was determined that polymorphic variations associated with an increased risk of low BMD existed in CETP, PROL4, GRID2, PDE4D and GPX3 nucleotide sequences. In the CETP locus, polymorphic variants at positions selected from the group consisting of rs7500979, rs2217332, rs8044804, rs2270835, rs2133783, rs247609, rs952440, rs881598, rs2291955, rs2518054, rs866038, rs1436425, rs173537, rs247611, rs166017, rs173538, rs193694, rs7205692, rs8048746, rs247618, rs183130, rs6499863, rs4783961, rs3816117, rs711752, rs708272, rs1864163, rs4369653, rs1864165, rs891141, rs891143, rs7205804, rs5885, rs1532625, rs1532624, rs289712, rs7499892, rs5883, rs289714, rs158480, rs289717, rs4344729, rs289718, rs289719, rs2033254, rs4784744, rs291044, rs8053613, rs5881, rs5880, rs7198026, rs5882, rs8045701, rs289741, rs1801706, rs289742, rs289743, rs289746, rs172337, rs289747, rs1566439, rs7205459, rs289749, rs289751, rs8059220, rs8058353, rs289735, rs289737, rs291042, rs1875236, rs821466, rs821465, rs4275846, rs289707, rs821463, rs289706, rs1167741, rs2052880, rs1167742, rs1183256, rs1651665, rs1651666, rs4784751, rs1651667, rs8052091, rs1684574, rs1684575, rs1672865, rs821470, rs1549669, rs291040 and rs289754 were tested for association with low BMD. Polymorphic variants at the following positions were associated with low BMD: rs166017, rs193694, rs7205804, rs1801706, rs7205459 and rs821465. At these positions in SEQ ID NO:1, a thymine at position 14328, a thymine at position 14996, a guanine at position 37336, a guanine at position 50109, a thymine at position 57618, and a guanine at position 68805 were associated with low BMD.

In the PROL4 locus, polymorphic variants at positions selected from the group consisting of rs523051, rs693620, rs2588349, rs2588350, rs619381, rs3759252, rs3759251, rs2418107, rs7303054, rs1838345, rs620878, rs2537817, rs1548803, rs667123, rs1838346, rs2159903, rs3944035, rs3741845, rs2110096, rs759055, rs589377, rs7960194, rs7978242, rs601051, rs4262797, rs2215714, rs1373434, rs2215715, rs612456, rs612808, rs689118, rs597468, rs592864, rs640372, rs7966559, rs654834, rs4763216, rs668521, rs669503, rs3906864, rs3906863, rs7957888, rs9300230, rs7306214, rs763839, rs2418105, rs666841, rs3851578, rs7138797, rs7295252, rs2418106, rs7299578, rs621112, rs3863320, rs1373432, rs1047699, rs1063193, rs2232959, rs2227296, rs1548804, rs2232958, rs2232957, rs2232956, rs1972571, rs3759250, rs3759249, rs1541525, rs2098248, rs2900550, rs7302130, rs4763583, rs4360778, rs1607695, rs1607694, rs2192139, rs7978300, rs7397871, rs4763217, rs2159900, rs10772370, rs7398682, rs2900551, rs2900552, rs2418214, rs2418215, rs965243, rs1117548, rs1520225, rs1520226, rs1520227, rs971919, rs2159901, rs2159902, rs2110099, rs7314847, rs7296003, rs4281556, rs4763219, rs3851579, rs3851580, rs1049119, rs2298866, rs2298865, rs2298864, rs2298863, rs3180393, rs2070837, rs7956204, rs2418216, rs3741844, rs4262798, rs2418217, rs2418218, rs7137492, rs2110100, rs1013312, rs4579993, rs1013313, rs7397106, rs2215716, rs2192140, rs4763589, rs1468697, rs2070837, rs3180393 and rs2298865 were tested for association with low BMD. Polymorphic variants at the following positions were associated with an increased risk of low BMD: rs2588350, rs619381, rs620878, rs759055, rs4262797, rs612808, rs3906863, rs7957888, rs763839, rs2418105, rs666841, rs3851578, rs7299578, rs621112, rs1047699, rs1548804, rs2232956, rs1520227 and rs2215716. At these positions in SEQ ID NO:2, a cytosine at position 2424, a cytosine at position 3625, a guanine at position 7097, an adenine at position 15688, a guanine at position 22861, a cytosine at position 24138, a cytosine at position 32459, an adenine at position 35151, a guanine at position 36930, an adenine at position 37490, a cytosine at position 38432, an adenine at position 38688, a guanine at position 42665, an adenine at position 43038, a cytosine at position 49075, an adenine at position 50773, an adenine at position 52107, a cytosine at position 75246, and a guanine at position 93715 were associated with risk of low BMD.

In the GRID2 locus, polymorphic variants at positions selected from the group consisting of rs1433661, rs1485009, rs7681947, rs1816432, rs1485018, rs1485017, rs7438397, rs6834311, rs1368717, rs1017391, rs2870701, rs7679839, rs1385404, rs1368716, rs4693316, rs1905707, rs1905708, rs1905709, rs3912442, rs2082553, rs6831638, rs5860329, rs2870702, rs2870703, rs1948016, rs6835836, rs1994253, rs1905710, rs1485019, rs978191, rs1385405, rs7694361, rs1905711, rs1905734, rs1485012, rs1485013, rs4692981, rs7670552, rs7670932, rs7688091, rs7440540, rs2171000, rs2870704, rs7655758, rs7661436, rs7662289, rs7667044, rs7691929, rs5860330, rs901013, rs901012, rs901011, rs1948018, rs2870705, rs1948017, rs1905733, rs1385408, rs1385409, rs1385410, rs1485026, rs1485027, rs2904483, rs1385406, rs1905732, rs2046418, rs2200377, rs1905731, rs1905730, rs975713, rs6820985, rs7670441, rs6810794, rs7676623, rs1154861, rs1032125, rs1485022, rs1485024, rs3913651, rs4693319, rs1872383, rs2200376, rs7668090, rs7692930, rs967096, rs6822249, rs6532405, rs1017897, rs7672674, rs7694568, rs2904484, rs7340830, rs1485033, rs2870706, rs1905729, rs4693320, rs6848749, rs6532406, rs6532407, rs1905728, rs6819866, rs1905727, rs7674069, rs1905724, rs1905723, rs1485020 and rs6814101 were tested for association with low BMD. Polymorphic variants at the following positions were associated with an increased risk of low BMD: rs1433661, rs7679839, rs1368716, rs1905707, rs1905708, rs1994253, rs1485019, rs1905734, rs1485012, rs7670552, rs7691929, rs1948018, rs1948017, rs1485024, rs7694568, rs4693320, rs6848749, rs6532406, rs6532407 and rs6819866. At these positions in SEQ ID NO:3, a thymine at position 206, a guanine at position 8612, an adenine at position 9285, a thymine at position 11866, a guanine at position 11958, a cytosine at position 28773, a thymine at position 29876, a cytosine at position 35588, a guanine at position 37663, a thymine at position 39375, a cytosine at position 43705, an adenine at position 48962, a cytosine at position 49110, an adenine at position 65050, a cytosine at position 78331, a thymine at position 85405, a guanine at position 86441, an adenine at position 86967, a cytosine at position 87121, and an adenine at position 90969 were associated with risk of low BMD.

In the PDE4D locus, polymorphic variants at positions selected from the group consisting of rs6886495, rs6450498, rs1472456, rs4700315, rs4700316, rs7714708, rs7710479, rs2968013, rs2968014, rs2968015, rs1391648, rs2055297, rs2055296, rs3989138, rs4700317, rs2036220, rs7727206, rs7723432, rs1546221, rs4479801, rs4395595, rs4395596, rs4699932, rs2936201, rs7356672, rs2936200, rs1909296, rs7703131, rs7445308, rs3087748, rs4321723, rs2968016, rs5868151, rs1874858, rs1874857, rs7712922, rs4631140, rs4469166, rs1078369, rs1078368, rs2968006, rs2968005, rs2936190, rs2409613, rs4415048, rs2968004, rs2968003, rs2968002, rs2936191, rs1498610, rs6874662, rs3060393, rs7729722, rs7733884, rs7714489, rs7735570, rs2936193, rs2291851, rs2291852, rs1498602, rs1995166, rs1498603, rs1498604, rs1498605, rs1948651, rs4699934, rs4700319, rs2279737, rs7720361, rs7706419, rs1006431, rs1353747, rs1498606, rs1353748, rs1553113, rs2968012, rs2968011, rs1498608, rs2936189, rs1498609, rs2968019, rs6891238, rs2968010, rs2968009, rs2936203, rs1498601, rs1498600, rs1498599, rs2936202, rs7730070, rs6450501, rs6450502, rs6889456, rs6894618, rs7706044, rs7707541, rs7712076, rs6892860, rs6867053, rs7737269, rs6864156, rs950447, rs2936196, rs7719347, rs1391649, rs1391650, rs1391651, rs1353749, rs10682149, rs5868153, rs1363882, rs2409626, rs2968018, rs954740, rs986067, rs6869400, and rs5010782 were tested for association with low BMD. Polymorphic variants at the following positions were associated with an increased risk of low BMD: rs7714708, rs1498602, rs4699934, rs1006431, rs1353747, rs1498608, rs1498609, rs2968010, rs2936202 and rs1391649. At these positions in SEQ ID NO:4, an adenine at position 1599, a cytosine at position 39626, a thymine at position 40356, a thymine at position 43555, a thymine at position 44066, a thymine at position 49652, a cytosine at position 51103, an adenine at position 57173, a guanine at position 63980, and an adenine at position 82591 were associated with risk of low BMD.

In the GPX3/TNIP1 locus, polymorphic variants at positions selected from the group consisting of rs1478398, rs1478397, rs1160114, rs1160113, rs1382323, rs1160112, rs7709870, rs7710643, rs7730467, rs6579829, rs6579830, rs6579831, rs6896232, rs1351131, rs1038074, rs1478396, rs6880512, rs4958858, rs4958431, rs4958432, rs6898463, rs4958859, rs4130064, rs4130065, rs4133119, rs4958860, rs4958861, rs4437356, rs4958868, rs1478400, rs6889375, rs1600159, rs6875892, rs4608909, rs2345000, rs4516840, rs2054440, rs707141, rs707142, rs841236, rs707143, rs70144, rs6869405, rs707145, rs707146, rs707148, rs707150, rs5872184, rs3763015, rs2042235, rs3763013, rs2042236, rs1946234, rs1946235, rs1946236, rs8177402, rs8177403, rs8177404, rs8177405, rs8177406, rs8177407, rs8177408, rs8177409, rs6888961, rs8177410, rs8177411, rs8177412, rs8177413, rs870407, rs870406, rs6873202, rs8177414, rs8177415, rs3805435, rs8177416, rs3792799, rs3792798, rs3828599, rs8177417, rs3792797, rs8177418, rs8177419, rs8177420, rs8177421, rs4958872, rs3792796, rs8177422, rs8177423, rs4958434, rs8177424, rs8177425, rs8177426, rs8177427, rs8177429, rs6889737, rs3792795, rs8177430, rs8177431, rs4958873, rs8177432, rs8177433, rs8177434, rs8177435, rs3763011, rs8177436, rs8177437, rs4958874, rs8177439, rs8177440, rs8177441, rs8177442, rs8177443, rs869975, rs869976, rs8177444, rs8177445, rs7721469, rs8177446, rs7704191, rs8177447, rs11548, rs2230303, rs7722386, rs8177448, rs8177449, rs2070593, rs8177450, rs8177451, rs8177452, rs8177453, rs8177454, rs3763010, rs8177455, rs8177456, rs736775, rs2277940, rs8177458, rs8177834, rs3924, rs2233312, rs2233311, rs2233310, rs2233309, rs4958875, rs2233308, rs2233307, rs2233306, rs2233305, rs2233304, rs2233303, rs2233302, rs2287719, rs2287720, rs7727034, rs7727250, rs7709800, rs3840312, rs2287721, rs6875293, rs3805434, rs2080982, rs2080983, rs2287722, rs2233301, rs2233300, rs4958876, rs2233299, rs2233298, rs2287723, rs2161359, rs7734456, rs4292439, rs4958878, rs6862024, rs3834819, rs2233297, rs2233296, rs2233295, rs2233294, rs7713028, rs7713223, rs7713567, rs888989, rs2233293, rs3749657, rs2233292, rs2112635, rs871269, rs3792794, rs6579837, rs3805433, rs5872186, rs2233291, rs2233290, rs2233289, rs4958435, rs4958880, rs1422673, rs2042234, rs3805432, rs3805431, rs2233288, rs2233287, rs3815720, rs3792792, rs3792791, rs2303018, rs3792790, rs4958436, rs2233286, rs2233285, rs7732451, rs2233284, rs1422674, rs3792789, rs4562032, rs6865077, rs1559126, rs3792788, rs1559127, rs3792786, rs6880110, rs6861227, rs3805430, rs1862364, rs4958881, rs3792785, rs6869605, rs6870205, rs4246047, rs4958882, rs3792784, rs3792783 and rs5872188 were tested for association with low BMD. Polymorphic variants at the following positions were associated with an increased risk of low BMD: rs1478398, rs1160114, rs1160113, rs1160112, rs4958858, rs4958431, rs6898463, rs4958859, rs4958860, rs4608909, rs707144, rs2042235, rs3763013, rs2042236, rs8177404, rs8177426, rs8177427, rs8177429, rs3792795, rs4958873, rs8177437, rs869975, rs8177447, rs11548, rs2277940, rs8177834, rs2233311, rs2233302, rs7727034, rs7727250, rs3805434, rs7734456, rs7713028, rs7713223, rs888989, rs3792794, rs4958880, rs1422673, rs3805432 and rs4958436. At these positions in SEQ ID NO:5, an adenine at position 231, a cytosine at position 582, a guanine at position 589, an adenine at position 1066, a thymine at position 5621, a guanine at position 5735, a cytosine at position 6658, a cytosine at position 7901, a thymine at position 15803, a cytosine at position 25599, a thymine at position 31203, a thymine at position 41624, a guanine at position 41671, an adenine at position 41825, a cytosine at position 43294, an adenine at position 46650, an adenine at position 46721, a guanine at position 46808, a guanine at position 47512, an adenine at position 47806, a guanine at position 49097, a guanine at position 50082, a thymine at position 51166, a cytosine at position 51493, a thymine at position 53187, an adenine at position 53699, a thymine at position 53929, a cytosine at position 58808, a cytosine at position 59187, a cytosine at position 59361, a cytosine at position 64049, a guanine at position 70882, an adenine at position 74131, a cytosine at position 74406, a cytosine at position 74740, a guanine at position 78432, a cytosine at position 82187, a cytosine at position 82698, an adenine at position 83214 and a thymine at position 86539 were associated with risk of low BMD.

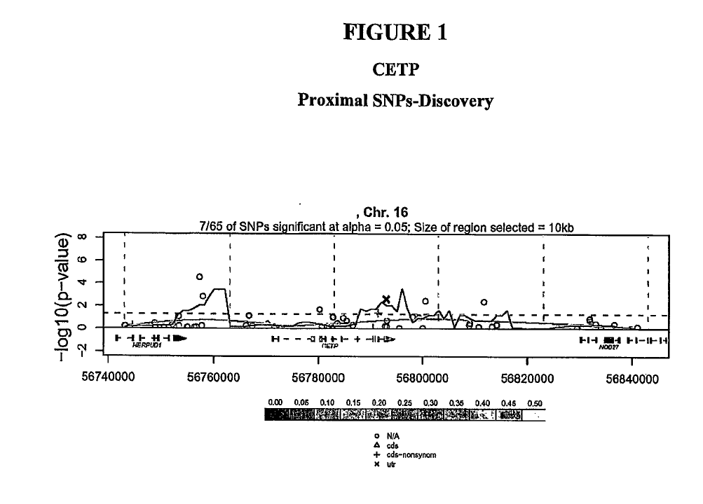

Based in part upon analyses summarized in FIGS. 1-5, regions with significant association have been identified in loci associated with low BMD. Any polymorphic variants associated with low BMD in a region of significant association can be utilized for embodiments described herein. The following table reports such regions, where “begin” and “end” designate the boundaries of the region according to chromosome positions within the genomic sequence provided in SEQ ID Nos:1-5. The locus, the chromosome on which the locus resides and an incident polymorphism in the locus also are noted.

TABLE 1
COMBINED ASSOCIATION RANGES

| Incident SNP | Locus | Chromosome | Begin | End | Size |
|---|---|---|---|---|---|
| rs1801706 | CETP | 16q21 | 14328 | 68805 | 54477 |
| rs1047699 | PROL4 | 12p13 | 2424 | 93715 | 91291 |
| rs1948017 | GRID2 | 4q22 | 206 | 90969 | 90763 |
| rs1498608 | PDE4D | 5q12 | 1599 | 82591 | 80992 |
| rs869975 | GPX3/TNIP1 | 5q23 | 231 | 86539 | 86308 |

Additional Polymorphic Variants Associated with Low BMD

Also provided is a method for identifying polymorphic variants proximal to an incident, founder polymorphic variant associated with low BMD. Thus, featured herein are methods for identifying a polymorphic variation associated with low BMD that is proximal to an incident polymorphic variation associated with low BMD, which comprises identifying a polymorphic variant proximal to the incident polymorphic variant associated with low BMD, where the incident polymorphic variant is in a CETP, PROL4, GRID2, PDE4D or GPX3/TNIP1 nucleotide sequence. The nucleotide sequence often comprises a polynucleotide sequence selected from the group consisting of (a) a polynucleotide sequence of SEQ ID NO's:1-5; (b) a polynucleotide sequence that encodes a polypeptide having an amino acid sequence encoded by a polynucleotide sequence of SEQ ID NO's:1-5; and (c) a polynucleotide sequence that encodes a polypeptide having an amino acid sequence that is 90% or more identical to an amino acid sequence encoded by a nucleotide sequence of SEQ ID NO's:1-5 or a polynucleotide sequence 90% or more identical to the polynucleotide sequence of SEQ ID NO's:1-5. The presence or absence of an association of the proximal polymorphic variant with low BMD then is determined using a known association method, such as a method described in the Examples hereafter. In an embodiment, the incident polymorphic variant is a polymorphic variant associated with low BMD described herein. In another embodiment, the proximal polymorphic variant identified sometimes is a publicly disclosed polymorphic variant, which for example, sometimes is published in a publicly available database. In other embodiments, the polymorphic variant identified is not publicly disclosed and is discovered using a known method, including, but not limited to, sequencing a region surrounding the incident polymorphic variant in a group of nucleic samples. Thus, multiple polymorphic variants proximal to an incident polymorphic variant are associated with low BMD using this method.

The proximal polymorphic variant often is identified in a region surrounding the incident polymorphic variant. In certain embodiments, this surrounding region is about 50 kb flanking the first polymorphic variant (e.g. about 50 kb 5′ of the first polymorphic variant and about 50 kb 3′ of the first polymorphic variant), and the region sometimes is composed of shorter flanking sequences, such as flanking sequences of about 40 kb, about 30 kb, about 25 kb, about 20 kb, about 15 kb, about 10 kb, about 7 kb, about 5 kb, or about 2 kb 5′ and 3′ of the incident polymorphic variant. In other embodiments, the region is composed of longer flanking sequences, such as flanking sequences of about 55 kb, about 60 kb, about 65 kb, about 70 kb, about 75 kb, about 80 kb, about 85 kb, about 90 kb, about 95 kb, or about 100 kb 5′ and 3′ of the incident polymorphic variant.

In certain embodiments, polymorphic variants associated with low BMD are identified iteratively. For example, a first proximal polymorphic variant is associated with low BMD using the methods described above and then another polymorphic variant proximal to the first proximal polymorphic variant is identified (e.g., publicly disclosed or discovered) and the presence or absence of an association of one or more other polymorphic variants proximal to the first proximal polymorphic variant with low BMD is determined.

The methods described herein are useful for identifying or discovering additional polymorphic variants that may be used to further characterize a gene, region or loci associated with a condition, a disease (e.g., osteoporosis), or a disorder. For example, allelotyping or genotyping data from the additional polymorphic variants may be used to identify a functional mutation or a region of linkage disequilibrium. In certain embodiments, polymorphic variants identified or discovered within a region comprising the first polymorphic variant associated with low BMD are genotyped using the genetic methods and sample selection techniques described herein, and it can be determined whether those polymorphic variants are in linkage disequilibrium with the first polymorphic variant. The size of the region in linkage disequilibrium with the first polymorphic variant also can be assessed using these genotyping methods. Thus, provided herein are methods for determining whether a polymorphic variant is in linkage disequilibrium with a first polymorphic variant associated with low BMD, and such information can be used in prognosis/diagnosis methods described herein.

Isolated Nucleic Acids

Featured herein are isolated CETP, PROL4, GRID2, PDE4D or GPX3/TNIP1 nucleic acid variants depicted in SEQ ID NO's:1-10, and substantially identical nucleic acids thereof. A nucleic acid variant may be represented on one or both strands in a double-stranded nucleic acid or on one chromosomal complement (heterozygous) or both chromosomal complements (homozygous)).

As used herein, the term “nucleic acid” includes DNA molecules (e.g., a complementary DNA (cDNA) and genomic DNA (gDNA)) and RNA molecules (e.g., mRNA, rRNA, siRNA and tRNA) and analogs of DNA or RNA, for example, by use of nucleotide analogs. The nucleic acid molecule can be single-stranded and it is often double-stranded. The term “isolated or purified nucleic acid” refers to nucleic acids that are separated from other nucleic acids present in the natural source of the nucleic acid. For example, with regard to genomic DNA, the term “isolated” includes nucleic acids which are separated from the chromosome with which the genomic DNA is naturally associated. An “isolated” nucleic acid is often free of sequences which naturally flank the nucleic acid (i.e., sequences located at the 5′ and/or 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb or 0.1 kb of 5′ and/or 3′ nucleotide sequences which flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized. As used herein, the term “gene” refers to a nucleotide sequence that encodes a polypeptide.

The nucleic acid often comprises a part of or all of a nucleotide sequence in SEQ ID NO's:1-5, or a substantially identical sequence thereof. Such a nucleotide sequence sometimes is a 5′ and/or 3′ sequence flanking a polymorphic variant described above that is 5-1000 nucleotides in length, or in some embodiments 5-500, 5-100, 5-75, 5-50, 5-45, 5-40, 5-35, 5-30, 5-25 or 5-20 nucleotides in length. Other embodiments are directed to methods of identifying a polymorphic variation at one or more positions in a nucleic acid (e.g., genotyping at one or more positions in the nucleic acid), such as at a position corresponding to rs1801706 in the CETP gene, rs1047699 in the PROL4 gene, rs1948017 in the GRID2 gene, rs1498608 in the PDE4D gene, or rs869975 in the GPX3 gene.

Also included herein are nucleic acid fragments. These fragments often are a nucleotide sequence identical to a nucleotide sequence of SEQ ID NO's:1-10, a nucleotide sequence substantially identical to a nucleotide sequence of SEQ ID NO's:1-10, or a nucleotide sequence that is complementary to the foregoing. The nucleic acid fragment may be identical, substantially identical or homologous to a nucleotide sequence in an exon or an intron in a nucleotide sequence of SEQ ID NO's:1-5, and may encode a domain or part of a domain of a polypeptide. Sometimes, the fragment will comprises one or more of the polymorphic variations described herein as being associated with low BMD. The nucleic acid fragment is often 50, 100, or 200 or fewer base pairs in length, and is sometimes about 300, 400, 500, 600, 700, 800, 900, 1000, 1100, 1200, 1300, 1400, 1500, 2000, 3000, 4000, 5000, 10000, 15000, or 20000 base pairs in length. A nucleic acid fragment that is complementary to a nucleotide sequence identical or substantially identical to a nucleotide sequence in SEQ ID NO's:1-5 and hybridizes to such a nucleotide sequence under stringent conditions is often referred to as a “probe.” Nucleic acid fragments often include one or more polymorphic sites, or sometimes have an end that is adjacent to a polymorphic site as described hereafter. CETP nucleic acid fragments sometimes encode the mature protein from positions 182 to 1609 of the mRNA sequence (SEQ ID NO: 6), for example.

An example of a nucleic acid fragment is an oligonucleotide. As used herein, the term “oligonucleotide” refers to a nucleic acid comprising about 8 to about 50 covalently linked nucleotides, often comprising from about 8 to about 35 nucleotides, and more often from about 10 to about 25 nucleotides. The backbone and nucleotides within an oligonucleotide may be the same as those of naturally occurring nucleic acids, or analogs or derivatives of naturally occurring nucleic acids, provided that oligonucleotides having such analogs or derivatives retain the ability to hybridize specifically to a nucleic acid comprising a targeted polymorphism. Oligonucleotides described herein may be used as hybridization probes or as components of prognostic or diagnostic assays, for example, as described herein.

Oligonucleotides are typically synthesized using standard methods and equipment, such as the ABI™3900 High Throughput DNA Synthesizer and the EXPEDITE™ 8909 Nucleic Acid Synthesizer, both of which are available from Applied Biosystems (Foster City, Calif.). Analogs and derivatives are exemplified in U.S. Pat. Nos. 4,469,863; 5,536,821; 5,541,306; 5,637,683; 5,637,684; 5,700,922; 5,717,083; 5,719,262; 5,739,308; 5,773,601; 5,886,165; 5,929,226; 5,977,296; 6,140,482; WO 00/56746; WO 01/14398, and related publications. Methods for synthesizing oligonucleotides comprising such analogs or derivatives are disclosed, for example, in the patent publications cited above and in U.S. Pat. Nos. 5,614,622; 5,739,314; 5,955,599; 5,962,674; 6,117,992; in WO 00/75372; and in related publications.

Oligonucleotides may also be linked to a second moiety. The second moiety may be an additional nucleotide sequence such as a tail sequence (e.g., a polyadenosine tail), an adapter sequence (e.g., phage M13 universal tail sequence), and others. Alternatively, the second moiety may be a non-nucleotide moiety such as a moiety which facilitates linkage to a solid support or a label to facilitate detection of the oligonucleotide. Such labels include, without limitation, a radioactive label, a fluorescent label, a chemiluminescent label, a paramagnetic label, and the like. The second moiety may be attached to any position of the oligonucleotide, provided the oligonucleotide can hybridize to the nucleic acid comprising the polymorphism.

Uses of Nucleic Acid Sequence

Nucleic acid coding sequences (e.g., SEQ ID NO: 7-12) may be used for diagnostic purposes for detection and control of polypeptide expression. Also, included herein are oligonucleotide sequences such as antisense RNA, small-interfering RNA (siRNA) and DNA molecules and ribozymes that function to inhibit translation of a polypeptide. Antisense techniques and RNA interference techniques are known in the art and are described herein.

Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. For example, hammerhead motif ribozyme molecules may be engineered that specifically and efficiently catalyze endonucleolytic cleavage of RNA sequences corresponding to or complementary to CETP, PROL4, GRID2, PDE4D or GPX3/TNIP1 nucleotide sequences. Specific ribozyme cleavage sites within any potential RNA target are initially identified by scanning the target molecule for ribozyme cleavage sites which include the following sequences, GUA, GUU and GUC. Once identified, short RNA sequences of between fifteen (15) and twenty (20) ribonucleotides corresponding to the region of the target gene containing the cleavage site may be evaluated for predicted structural features such as secondary structure that may render the oligonucleotide sequence unsuitable. The suitability of candidate targets may also be evaluated by testing their accessibility to hybridization with complementary oligonucleotides, using ribonuclease protection assays.

Antisense RNA and DNA molecules, siRNA and ribozymes may be prepared by any method known in the art for the synthesis of RNA molecules. These include techniques for chemically synthesizing oligodeoxyribonucleotides well known in the art such as solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors which incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

DNA encoding a polypeptide also may have a number of uses for the diagnosis of diseases, including low BMD, resulting from aberrant expression of a target gene described herein. For example, the nucleic acid sequence may be used in hybridization assays of biopsies or autopsies to diagnose abnormalities of expression or function (e.g., Southern or Northern blot analysis, in situ hybridization assays).

In addition, the expression of a polypeptide during embryonic development may also be determined using nucleic acid encoding the polypeptide. As addressed, infra, production of functionally impaired polypeptide is the cause of various disease states, such as osteoporosis. In situ hybridizations using polypeptide as a probe may be employed to predict problems related to low BMD. Further, as indicated, infra, administration of human active polypeptide, recombinantly produced as described herein, may be used to treat disease states related to functionally impaired polypeptide. Alternatively, gene therapy approaches may be employed to remedy deficiencies of functional polypeptide or to replace or compete with dysfunctional polypeptide.

Expression Vectors, Host Cells, and Genetically Engineered Cells

Provided herein are nucleic acid vectors, often expression vectors, which contain a CETP, PROL4, GRID2, PDE4D or GPX3/TNIP1 nucleotide sequence or a substantially identical sequence thereof. As used herein, the term “vector” refers to a nucleic acid molecule capable of transporting another nucleic acid to which it has been linked and can include a plasmid, cosmid, or viral vector. The vector can be capable of autonomous replication or it can integrate into a host DNA. Viral vectors may include replication defective retroviruses, adenoviruses and adeno-associated viruses for example.

A vector can include a CETP, PROL4, GRID2, PDE4D or GPX3/TNIP1 nucleotide sequence in a form suitable for expression of an encoded target polypeptide or target nucleic acid in a host cell. A “target polypeptide” is a polypeptide encoded by a CETP, PROL4, GRID2, PDE4D or GPX3/TNIP1 nucleotide sequence or a substantially identical nucleotide sequence thereof. The recombinant expression vector typically includes one or more regulatory sequences operatively linked to the nucleic acid sequence to be expressed. The term “regulatory sequence” includes promoters, enhancers and other expression control elements (e.g., polyadenylation signals). Regulatory sequences include those that direct constitutive expression of a nucleotide sequence, as well as tissue-specific regulatory and/or inducible sequences. The design of the expression vector can depend on such factors as the choice of the host cell to be transformed, the level of expression of polypeptide desired, and the like. Expression vectors can be introduced into host cells to produce target polypeptides, including fusion polypeptides.

Recombinant expression vectors can be designed for expression of target polypeptides in prokaryotic or eukaryotic cells. For example, target polypeptides can be expressed in E. coli, insect cells (e.g., using baculovirus expression vectors), yeast cells, or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of polypeptides in prokaryotes is most often carried out in E. coli with vectors containing constitutive or inducible promoters directing the expression of either fusion or non-fusion polypeptides. Fusion vectors add a number of amino acids to a polypeptide encoded therein, usually to the amino terminus of the recombinant polypeptide. Such fusion vectors typically serve three purposes: 1) to increase expression of recombinant polypeptide; 2) to increase the solubility of the recombinant polypeptide; and 3) to aid in the purification of the recombinant polypeptide by acting as a ligand in affinity purification. Often, a proteolytic cleavage site is introduced at the junction of the fusion moiety and the recombinant polypeptide to enable separation of the recombinant polypeptide from the fusion moiety subsequent to purification of the fusion polypeptide. Such enzymes, and their cognate recognition sequences, include Factor Xa, thrombin and enterokinase. Typical fusion expression vectors include pGEX (Pharmacia Biotech Inc; Smith & Johnson, Gene 67: 31-40 (1988)), pMAL (New England Biolabs, Beverly, Mass.) and pRIT5 (Pharmacia, Piscataway, N.J.) which fuse glutathione S-transferase (GST), maltose E binding polypeptide, or polypeptide A, respectively, to the target recombinant polypeptide.

Purified fusion polypeptides can be used in screening assays and to generate antibodies specific for target polypeptides. In a therapeutic embodiment, fusion polypeptide expressed in a retroviral expression vector is used to infect bone marrow cells that are subsequently transplanted into irradiated recipients. The pathology of the subject recipient is then examined after sufficient time has passed (e.g., six (6) weeks).

Expressing the polypeptide in host bacteria with an impaired capacity to proteolytically cleave the recombinant polypeptide is often used to maximize recombinant polypeptide expression (Gottesman, S., Gene Expression Technology: Methods in Enzymology, Academic Press, San Diego, Calif. 185: 119-128 (1990)). Another strategy is to alter the nucleotide sequence of the nucleic acid to be inserted into an expression vector so that the individual codons for each amino acid are those preferentially utilized in E. coli (Wada et al., Nucleic Acids Res. 20: 2111-2118 (1992)). Such alteration of nucleotide sequences can be carried out by standard DNA synthesis techniques.

When used in mammalian cells, the expression vector's control functions are often provided by viral regulatory elements. For example, commonly used promoters are derived from polyoma, Adenovirus 2, cytomegalovirus and Simian Virus 40. Recombinant mammalian expression vectors are often capable of directing expression of the nucleic acid in a particular cell type (e.g., tissue-specific regulatory elements are used to express the nucleic acid). Non-limiting examples of suitable tissue-specific promoters include an albumin promoter (liver-specific; Pinkert et al., Genes Dev. 1: 268-277 (1987)), lymphoid-specific promoters (Calame & Eaton, Adv. Immunol. 43: 235-275 (1988)), promoters of T cell receptors (Winoto & Baltimore, EMBO J. 8: 729-733 (1989)) promoters of immunoglobulins (Banerji et al., Cell 33: 729-740 (1983); Queen & Baltimore, Cell 33: 741-948 (1983)), neuron-specific promoters (e.g., the neurofilament promoter; Byrne & Ruddle, Proc. Natl. Acad. Sci. USA 86: 5473-5477 (1989)), pancreas-specific promoters (Edlund et al., Science 230: 912-916 (1985)), and mammary gland-specific promoters (e.g., milk whey promoter; U.S. Pat. No. 4,873,316 and European Application Publication No. 264,166). Developmentally-regulated promoters are sometimes utilized, for example, the murine hox promoters (Kessel & Gruss, Science 249: 374-379 (1990)) and the α-fetopolypeptide promoter (Campes & Tilghman, Genes Dev. 3: 537-546 (1989)).

A CETP, PROL4, GRID2, PDE4D or GPX3/TNIP1 nucleic acid may also be cloned into an expression vector in an antisense orientation. Regulatory sequences (e.g., viral promoters and/or enhancers) operatively linked to a CETP, PROL4, GRID2, PDE4D or GPX3/TNIP1 nucleic acid cloned in the antisense orientation can be chosen for directing constitutive, tissue specific or cell type specific expression of antisense RNA in a variety of cell types. Antisense expression vectors can be in the form of a recombinant plasmid, phagemid or attenuated virus. For a discussion of the regulation of gene expression using antisense genes see, e.g., Weintraub et al., Antisense RNA as a molecular tool for genetic analysis, Reviews—Trends in Genetics, Vol. 1(1) (1986).

Also provided herein are host cells that include a CETP, PROL4, GRID2, PDE4D or GPX3/TNIP1 nucleotide sequence within a recombinant expression vector or a fragment of such a nucleotide sequence which facilitate homologous recombination into a specific site of the host cell genome. The terms “host cell” and “recombinant host cell” are used interchangeably herein. Such terms refer not only to the particular subject cell but rather also to the progeny or potential progeny of such a cell. Because certain modifications may occur in succeeding generations due to either mutation or environmental influences, such progeny may not, in fact, be identical to the parent cell, but are still included within the scope of the term as used herein. A host cell can be any prokaryotic or eukaryotic cell. For example, a target polypeptide can be expressed in bacterial cells such as E. coli, insect cells, yeast or mammalian cells (such as Chinese hamster ovary cells (CHO) or COS cells). Other suitable host cells are known to those skilled in the art.

Vectors can be introduced into host cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, transduction/infection, DEAE-dextran-mediated transfection, lipofection, or electroporation.

A host cell provided herein can be used to produce (i.e., express) a target polypeptide or a substantially identical polypeptide thereof. Accordingly, further provided are methods for producing a target polypeptide using host cells described herein. In one embodiment, the method includes culturing host cells into which a recombinant expression vector encoding a target polypeptide has been introduced in a suitable medium such that a target polypeptide is produced. In another embodiment, the method further includes isolating a target polypeptide from the medium or the host cell.

Also provided are cells or purified preparations of cells which include a CETP, PROL4, GRID2, PDE4D or GPX3/TNIP1 transgene, or which otherwise misexpress target polypeptide. Cell preparations can consist of human or non-human cells, e.g., rodent cells, e.g., mouse or rat cells, rabbit cells, or pig cells. In preferred embodiments, the cell or cells include a CETP, PROL4, GRID2, PDE4D or GPX3/TNIP1 transgene (e.g., a heterologous form of a CETP, PROL4, GRID2, PDE4D or GPX3/TNIP1 gene, such as a human gene expressed in non-human cells). The transgene can be misexpressed, e.g., overexpressed or underexpressed. In other preferred embodiments, the cell or cells include a gene which misexpress an endogenous target polypeptide (e.g., expression of a gene is disrupted, also known as a knockout). Such cells can serve as a model for studying disorders which are related to mutated or mis-expressed alleles or for use in drug screening. Also provided are human cells (e.g., a hematopoietic stem cells) transformed with a CETP, PROL4, GRID2, PDE4D or GPX3/TNIP1 nucleic acid.

Also provided are cells or a purified preparation thereof (e.g., human cells) in which an endogenous CETP, PROL4, GRID2, PDE4D or GPX3/TNIP1 nucleic acid is under the control of a regulatory sequence that does not normally control the expression of the endogenous gene corresponding to a CETP, PROL4, GRID2, PDE4D or GPX3/TNIP1 nucleotide sequence. The expression characteristics of an endogenous gene within a cell (e.g., a cell line or microorganism) can be modified by inserting a heterologous DNA regulatory element into the genome of the cell such that the inserted regulatory element is operably linked to the corresponding endogenous gene. For example, an endogenous corresponding gene (e.g., a gene which is “transcriptionally silent,” not normally expressed, or expressed only at very low levels) may be activated by inserting a regulatory element which is capable of promoting the expression of a normally expressed gene product in that cell. Techniques such as targeted homologous recombinations, can be used to insert the heterologous DNA as described in, e.g., Chappel, U.S. Pat. No. 5,272,071; WO 91/06667, published on May 16, 1991.

Transyenic Animals

Non-human transgenic animals that express a heterologous target polypeptide (e.g., expressed from a CETP, PROL4, GRID2, PDE4D or GPX3/TNIP1 nucleic acid or substantially identical sequence thereof) can be generated. Such animals are useful for studying the function and/or activity of a target polypeptide and for identifying and/or evaluating modulators of the activity of CETP, PROL4, GRID2, PDE4D or GPX3/TNIP1 nucleic acids and encoded polypeptides. As used herein, a “transgenic animal” is a non-human animal such as a mammal (e.g., a non-human primate such as chimpanzee, baboon, or macaque; an ungulate such as an equine, bovine, or caprine; or a rodent such as a rat, a mouse, or an Israeli sand rat), a bird (e.g., a chicken or a turkey), an amphibian (e.g., a frog, salamander, or newt), or an insect (e.g., Drosophila melanogaster), in which one or more of the cells of the animal includes a transgene. A transgene is exogenous DNA or a rearrangement (e.g., a deletion of endogenous chromosomal DNA) that is often integrated into or occurs in the genome of cells in a transgenic animal. A transgene can direct expression of an encoded gene product in one or more cell types or tissues of the transgenic animal, and other transgenes can reduce expression (e.g., a knockout). Thus, a transgenic animal can be one in which an endogenous nucleic acid homologous to a CETP, PROL4, GRID2, PDE4D or GPX3/TNIP1 nucleic acid has been altered by homologous recombination between the endogenous gene and an exogenous DNA molecule introduced into a cell of the animal (e.g., an embryonic cell of the animal) prior to development of the animal.

Intronic sequences and polyadenylation signals can also be included in the transgene to increase expression efficiency of the transgene. One or more tissue-specific regulatory sequences can be operably linked to a CETP, PROL4, GRID2, PDE4D or GPX3/TNIP1 nucleotide sequence to direct expression of an encoded polypeptide to particular cells. A transgenic founder animal can be identified based upon the presence of a CETP, PROL4, GRID2, PDE4D or GPX3/TNIP1 nucleotide sequence in its genome and/or expression of encoded mRNA in tissues or cells of the animals. A transgenic founder animal can then be used to breed additional animals carrying the transgene. Moreover, transgenic animals carrying a CETP, PROL4, GRID2, PDE4D or GPX3/TNIP1 nucleotide sequence can further be bred to other transgenic animals carrying other transgenes.

Target polypeptides can be expressed in transgenic animals or plants by introducing, for example, a CETP, PROL4, GRID2, PDE4D or GPX3/TNIP1 nucleic acid into the genome of an animal that encodes the target polypeptide. In preferred embodiments the nucleic acid is placed under the control of a tissue specific promoter, e.g., a milk or egg specific promoter, and recovered from the milk or eggs produced by the animal. Also included is a population of cells from a transgenic animal.

Target Polypeptides

Also featured herein are isolated target polypeptides, which are encoded by a CETP, PROL4, GRID2, PDE4D or GPX3/TNIP1 nucleotide sequence (e.g., SEQ ID NO's:1-10) or a substantially identical nucleotide sequence thereof, such as the polypeptides having amino acid sequences in SEQ ID NO's:11-15. The term “polypeptide” as used herein includes proteins and peptides. An “isolated” or “purified” polypeptide or protein is substantially free of cellular material or other contaminating proteins from the cell or tissue source from which the protein is derived, or substantially free from chemical precursors or other chemicals when chemically synthesized. In one embodiment, the language “substantially free” means preparation of a target polypeptide having less than about 30%, 20%, 10% and more preferably 5% (by dry weight), of non-target polypeptide (also referred to herein as a “contaminating protein”), or of chemical precursors or non-target chemicals. When the target polypeptide or a biologically active portion thereof is recombinantly produced, it is also preferably substantially free of culture medium, specifically, where culture medium represents less than about 20%, sometimes less than about 10%, and often less than about 5% of the volume of the polypeptide preparation. Isolated or purified target polypeptide preparations are sometimes 0.01 milligrams or more or 0.1 milligrams or more, and often 1.0 milligrams or more and 10 milligrams or more in dry weight.

Further included herein are target polypeptide fragments. The polypeptide fragment may be a domain or part of a domain of a target polypeptide. In addition, the polypeptide fragment may be a full-length polypeptide or a mature polypeptide (i.e., the polypeptide minus the signal peptide). For example, a fragment sometimes is a CTEP mature protein that corresponds to amino acid positions 18493 of SEQ ID NO:11. The polypeptide fragment may have increased, decreased or unexpected biological activity. The polypeptide fragment is often 50 or fewer, 100 or fewer, or 200 or fewer amino acids in length, and is sometimes 300, 400, 500, 600, 700, or 900 or fewer amino acids in length.

Substantially identical target polypeptides may depart from the amino acid sequences of target polypeptides in different manners. For example, conservative amino acid modifications may be introduced at one or more positions in the amino acid sequences of target polypeptides. A “conservative amino acid substitution” is one in which the amino acid is replaced by another amino acid having a similar structure and/or chemical function. Families of amino acid residues having similar structures and functions are well known. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Also, essential and non-essential amino acids may be replaced. A “non-essential” amino acid is one that can be altered without abolishing or substantially altering the biological function of a target polypeptide, whereas altering an “essential” amino acid abolishes or substantially alters the biological function of a target polypeptide. Amino acids that are conserved among target polypeptides are typically essential amino acids.

Also, target polypeptides may exist as chimeric or fusion polypeptides. As used herein, a target “chimeric polypeptide” or target “fusion polypeptide” includes a target polypeptide linked to a non-target polypeptide. A “non-target polypeptide” refers to a polypeptide having an amino acid sequence corresponding to a polypeptide which is not substantially identical to the target polypeptide, which includes, for example, a polypeptide that is different from the target polypeptide and derived from the same or a different organism. The target polypeptide in the fusion polypeptide can correspond to an entire or nearly entire target polypeptide or a fragment thereof. The non-target polypeptide can be fused to the N-terminus or C-terminus of the target polypeptide.

Fusion polypeptides can include a moiety having high affinity for a ligand. For example, the fusion polypeptide can be a GST-target fusion polypeptide in which the target sequences are fused to the C-terminus of the GST sequences, or a polyhistidine-target fusion polypeptide in which the target polypeptide is fused at the N- or C-terminus to a string of histidine residues. Such fusion polypeptides can facilitate purification of recombinant target polypeptide. Expression vectors are commercially available that already encode a fusion moiety (e.g., a GST polypeptide), and a nucleotide sequence in SEQ ID NO's:1-10, or a substantially identical nucleotide sequence thereof, can be cloned into an expression vector such that the fusion moiety is linked in-frame to the target polypeptide. Further, the fusion polypeptide can be a target polypeptide containing a heterologous signal sequence at its N-terminus. In certain host cells (e.g., mammalian host cells), expression, secretion, cellular internalization, and cellular localization of a target polypeptide can be increased through use of a heterologous signal sequence. Fusion polypeptides can also include all or a part of a serum polypeptide (e.g., an IgG constant region or human serum albumin).

Target polypeptides can be incorporated into pharmaceutical compositions and administered to a subject in vivo. Administration of these target polypeptides can be used to affect the bioavailability of a substrate of the target polypeptide and may effectively increase target polypeptide biological activity in a cell. Target fusion polypeptides may be useful therapeutically for the treatment of disorders caused by, for example, (i) aberrant modification or mutation of a gene encoding a target polypeptide; (ii) mis-regulation of the gene encoding the target polypeptide; and (iii) aberrant post-translational modification of a target polypeptide. Also, target polypeptides can be used as immunogens to produce anti-target antibodies in a subject, to purify target polypeptide ligands or binding partners, and in screening assays to identify molecules which inhibit or enhance the interaction of a target polypeptide with a substrate.

In addition, polypeptides can be chemically synthesized using techniques known in the art (See, e.g., Creighton, 1983 Proteins. New York, N.Y.: W. H. Freeman and Company, and Hunkapiller et al., (1984) Nature July 12-18; 310(5973):105-11). For example, a relative short fragment can be synthesized by use of a peptide synthesizer. Furthermore, if desired, non-classical amino acids or chemical amino acid analogs can be introduced as a substitution or addition into the fragment sequence. Non-classical amino acids include, but are not limited to, to the D-isomers of the common amino acids, 2,4-diaminobutyric acid, a-amino isobutyric acid, 4-aminobutyric acid, Abu, 2-amino butyric acid, g-Abu, e-Ahx, 6-amino hexanoic acid, Aib, 2-amino isobutyric acid, 3-amino propionic acid, ornithine, norleucine, norvaline, hydroxyproline, sarcosine, citrulline, homocitrulline, cysteic acid, t-butylglycine, t-butylalanine, phenylglycine, cyclohexylalanine, b-alanine, fluoroamino acids, designer amino acids such as b-methyl amino acids, Ca-methyl amino acids, Na-methyl amino acids, and amino acid analogs in general. Furthermore, the amino acid can be D (dextrorotary) or L (levorotary).

Polypeptides and polypeptide fragments sometimes are differentially modified during or after translation, e.g., by glycosylation, acetylation, phosphorylation, amidation, derivatization by known protecting/blocking groups, proteolytic cleavage, linkage to an antibody molecule or other cellular ligand, etc. Any of numerous chemical modifications may be carried out by known techniques, including but not limited, to specific chemical cleavage by cyanogen bromide, trypsin, chymotrypsin, papain, V8 protease, NaBH4; acetylation, formylation, oxidation, reduction; metabolic synthesis in the presence of tunicamycin; and the like. Additional post-translational modifications include, for example, N-linked or O-linked carbohydrate chains, processing of N-terminal or C-terminal ends), attachment of chemical moieties to the amino acid backbone, chemical modifications of N-linked or O-linked carbohydrate chains, and addition or deletion of an N-terminal methionine residue as a result of prokaryotic host cell expression. The polypeptide fragments may also be modified with a detectable label, such as an enzymatic, fluorescent, isotopic or affinity label to allow for detection and isolation of the polypeptide.

Also provided are chemically modified derivatives of polypeptides that can provide additional advantages such as increased solubility, stability and circulating time of the polypeptide, or decreased immunogenicity (see e.g., U.S. Pat. No. 4,179,337. The chemical moieties for derivitization may be selected from water soluble polymers such as polyethylene glycol, ethylene glycol/propylene glycol copolymers, carboxymethylcellulose, dextran, polyvinyl alcohol and the like. The polypeptides may be modified at random positions within the molecule, or at predetermined positions within the molecule and may include one, two, three or more attached chemical moieties.

The polymer may be of any molecular weight, and may be branched or unbranched. For polyethylene glycol, the preferred molecular weight is between about 1 kDa and about 100 kDa (the term “about” indicating that in preparations of polyethylene glycol, some molecules will weigh more, some less, than the stated molecular weight) for ease in handling and manufacturing. Other sizes may be used, depending on the desired therapeutic profile (e.g., the duration of sustained release desired, the effects, if any on biological activity, the ease in handling, the degree or lack of antigenicity and other known effects of the polyethylene glycol to a therapeutic protein or analog).

The polymers should be attached to the polypeptide with consideration of effects on functional or antigenic domains of the polypeptide. There are a number of attachment methods available to those skilled in the art (e.g., EP 0 401 384 (coupling PEG to G-CSF) and Malik et al. (1992) Exp Hematol. September; 20(8):1028-35 (pegylation of GM-CSF using tresyl chloride)). For example, polyethylene glycol may be covalently bound through amino acid residues via a reactive group, such as a free amino or carboxyl group. Reactive groups are those to which an activated polyethylene glycol molecule may be bound. The amino acid residues having a free amino group may include lysine residues and the N-terminal amino acid residues; those having a free carboxyl group may include aspartic acid residues, glutamic acid residues and the C-terminal amino acid residue. Sulfhydryl groups may also be used as a reactive group for attaching the polyethylene glycol molecules. For therapeutic purposes, the attachment sometimes is at an amino group, such as attachment at the N-terminus or lysine group.

Proteins can be chemically modified at the N-terminus. Using polyethylene glycol as an illustration of such a composition, one may select from a variety of polyethylene glycol molecules (by molecular weight, branching, and the like), the proportion of polyethylene glycol molecules to protein (polypeptide) molecules in the reaction mix, the type of pegylation reaction to be performed, and the method of obtaining the selected N-terminally pegylated protein. The method of obtaining the N-terminally pegylated preparation (i.e., separating this moiety from other monopegylated moieties if necessary) may be by purification of the N-terminally pegylated material from a population of pegylated protein molecules. Selective proteins chemically modified at the N-terminus may be accomplished by reductive alkylation, which exploits differential reactivity of different types of primary amino groups (lysine versus the N-terminal) available for derivatization in a particular protein. Under the appropriate reaction conditions, substantially selective derivatization of the protein at the N-terminus with a carbonyl group containing polymer is achieved.

Substantially Identical Nucleic Acids and Polypeptides

Nucleotide sequences and polypeptide sequences that are substantially identical to a CETP, PROL4, GRID2, PDE4D or GPX3/TNIP1 nucleotide sequence and the target polypeptide sequences encoded by those nucleotide sequences, respectively, are included herein. The term “substantially identical” as used herein refers to two or more nucleic acids or polypeptides sharing one or more identical nucleotide sequences or polypeptide sequences, respectively. Included are nucleotide sequences or polypeptide sequences that are 55% or more, 60% or more, 65% or more, 70% or more, 75% or more, 80% or more, 85% or more, 90% or more, 95% or more (each often within a 1%, 2%, 3% or 4% variability) identical to a CETP, PROL4, GRID2, PDE4D or GPX3/TNIP1 nucleotide sequence or the encoded target polypeptide amino acid sequences. One test for determining whether two nucleic acids are substantially identical is to determine the percent of identical nucleotide sequences or polypeptide sequences shared between the nucleic acids or polypeptides.

Calculations of sequence identity are often performed as follows. Sequences are aligned for optimal comparison purposes (e.g., gaps can be introduced in one or both of a first and a second amino acid or nucleic acid sequence for optimal alignment and non-homologous sequences can be disregarded for comparison purposes). The length of a reference sequence aligned for comparison purposes is sometimes 30% or more, 40% or more, 50% or more, often 60% or more, and more often 70% or more, 80% or more, 90% or more, or 100% of the length of the reference sequence. The nucleotides or amino acids at corresponding nucleotide or polypeptide positions, respectively, are then compared among the two sequences. When a position in the first sequence is occupied by the same nucleotide or amino acid as the corresponding position in the second sequence, the nucleotides or amino acids are deemed to be identical at that position. The percent identity between the two sequences is a function of the number of identical positions shared by the sequences, taking into account the number of gaps, and the length of each gap, introduced for optimal alignment of the two sequences.

Comparison of sequences and determination of percent identity between two sequences can be accomplished using a mathematical algorithm. Percent identity between two amino acid or nucleotide sequences can be determined using the algorithm of Meyers & Miller, CABIOS 4: 11-17 (1989), which has been incorporated into the ALIGN program (version 2.0), using a PAM120 weight residue table, a gap length penalty of 12 and a gap penalty of 4. Also, percent identity between two amino acid sequences can be determined using the Needleman & Wunsch, J. Mol. Biol. 48: 444-453 (1970) algorithm which has been incorporated into the GAP program in the GCG software package (available at the http address www.gcg.com), using either a Blossum 62 matrix or a PAM250 matrix, and a gap weight of 16, 14, 12, 10, 8, 6, or 4 and a length weight of 1, 2, 3, 4, 5, or 6. Percent identity between two nucleotide sequences can be determined using the GAP program in the GCG software package (available at http address www.gcg.com), using a NWSgapdna.CMP matrix and a gap weight of 40, 50, 60, 70, or 80 and a length weight of 1, 2, 3, 4, 5, or 6. A set of parameters often used is a Blossum 62 scoring matrix with a gap open penalty of 12, a gap extend penalty of 4, and a frameshift gap penalty of 5.

Another manner for determining if two nucleic acids are substantially identical is to assess whether a polynucleotide homologous to one nucleic acid will hybridize to the other nucleic acid under stringent conditions. As use herein, the term “stringent conditions” refers to conditions for hybridization and washing. Stringent conditions are known to those skilled in the art and can be found in Current Protocols in Molecular Biology, John Wiley & Sons, N.Y., 6.3.1-6.3.6 (1989). Aqueous and non-aqueous methods are described in that reference and either can be used. An example of stringent hybridization conditions is hybridization in 6× sodium chloride/sodium citrate (SSC) at about 45° C., followed by one or more washes in 0.2×SSC, 0.1% SDS at 50° C. Another example of stringent hybridization conditions are hybridization in 6× sodium chloride/sodium citrate (SSC) at about 45° C., followed by one or more washes in 0.2×SSC, 0.1% SDS at 55° C. A further example of stringent hybridization conditions is hybridization in 6× sodium chloride/sodium citrate (SSC) at about 45° C., followed by one or more washes in 0.2×SSC, 0.1% SDS at 60° C. Often, stringent hybridization conditions are hybridization in 6× sodium chloride/sodium citrate (SSC) at about 45° C., followed by one or more washes in 0.2×SSC, 0.1% SDS at 65° C. More often, stringency conditions are 0.5M sodium phosphate, 7% SDS at 65° C., followed by one or more washes at 0.2×SSC, 1% SDS at 65° C.

An example of a substantially identical nucleotide sequence to a nucleotide sequence in SEQ ID NO's:1-10 is one that has a different nucleotide sequence but still encodes the same polypeptide sequence encoded by the nucleotide sequence in SEQ ID NO's:1-10. Another example is a nucleotide sequence that encodes a polypeptide having a polypeptide sequence that is more than 70% or more identical to, sometimes more than 75% or more, 80% or more, or 85% or more identical to, and often more than 90% or more and 95% or more identical to a polypeptide sequence encoded by a nucleotide sequence in SEQ ID NO's:1-10. As used herein, “SEQ ID NO's:1-10” typically refers to one or more sequences in SEQ ID NO: 1, 2, 3, 4, 5, 6, 7, 8, 9, and/or 10. Many of the embodiments described herein are applicable to (a) a nucleotide sequence of SEQ ID NO's:1-10; (b) a nucleotide sequence which encodes a polypeptide consisting of an amino acid sequence encoded by a nucleotide sequence of SEQ ID NO's:1-10; (c) a nucleotide sequence which encodes a polypeptide that is 90% or more identical to an amino acid sequence encoded by a nucleotide sequence of SEQ ID NO's:1-10, or a nucleotide sequence about 90% or more identical to a nucleotide sequence of SEQ ID NO's:1-10; (d) a fragment of a nucleotide sequence of (a), (b), or (c); and/or a nucleotide sequence complementary to the nucleotide sequences of (a), (b), (c) and/or (d), where nucleotide sequences of (b) and (c), fragments of (b) and (c) and nucleotide sequences complementary to (b) and (c) are examples of substantially identical nucleotide sequences. Examples of substantially identical nucleotide sequences include nucleotide sequences from subjects that differ by naturally occurring genetic variance, which sometimes is referred to as background genetic variance (e.g. nucleotide sequences differing by natural genetic variance sometimes are 90%, 91%, 92%, 93%, 94%, 95%, 96%, 97%, 98%, or 99% identical to one another).

Nucleotide sequences in SEQ ID NO's:1-10 and amino acid sequences of encoded polypeptides can be used as “query sequences” to perform a search against public databases to identify other family members or related sequences, for example. Such searches can be performed using the NBLAST and XBLAST programs (version 2.0) of Altschul et al., J. Mol. Biol. 215: 403-10 (1990). BLAST nucleotide searches can be performed with the NBLAST program, score=100, wordlength=12 to obtain nucleotide sequences homologous to nucleotide sequences in SEQ ID NO's:1-10. BLAST polypeptide searches can be performed with the XBLAST program, score=50, wordlength=3 to obtain amino acid sequences homologous to polypeptides encoded by the nucleotide sequences of SEQ ID NO's:1-10. To obtain gapped alignments for comparison purposes, Gapped BLAST can be utilized as described in Altschul et al., Nucleic Acids Res. 25(17): 3389-3402 (1997). When utilizing BLAST and Gapped BLAST programs, default parameters of the respective programs (e.g., XBLAST and NBLAST) can be used (see the http address www.ncbi.nlm.nih.gov).

A nucleic acid that is substantially identical to a nucleotide sequence in SEQ ID NO's:1-10 may include polymorphic sites at positions equivalent to those described herein when the sequences are aligned. For example, using the alignment procedures described herein, SNPs in a sequence substantially identical to a sequence in SEQ ID NO's:1-10 can be identified at nucleotide positions that match with or correspond to (i.e., align) nucleotides at SNP positions in each nucleotide sequence in SEQ ID NO's:1-10. Also, where a polymorphic variation results in an insertion or deletion, insertion or deletion of a nucleotide sequence from a reference sequence can change the relative positions of other polymorphic sites in the nucleotide sequence.

Substantially identical nucleotide and polypeptide sequences include those that are naturally occurring, such as allelic variants (same locus), splice variants, homologs (different locus), and orthologs (different organism) or can be non-naturally occurring. Non-naturally occurring variants can be generated by mutagenesis techniques, including those applied to polynucleotides, cells, or organisms. The variants can contain nucleotide substitutions, deletions, inversions and insertions. Variation can occur in either or both the coding and non-coding regions. The variations can produce both conservative and non-conservative amino acid substitutions (as compared in the encoded product). Orthologs, homologs, allelic variants, and splice variants can be identified using methods known in the art. These variants normally comprise a nucleotide sequence encoding a polypeptide that is 50% or more, about 55% or more, often about 70-75% or more or about 80-85% or more, and sometimes about 90-95% or more identical to the amino acid sequences of target polypeptides or a fragment thereof. Such nucleic acid molecules can readily be identified as being able to hybridize under stringent conditions to a nucleotide sequence in SEQ ID NO's:1-10 or a fragment of this sequence. Nucleic acid molecules corresponding to orthologs, homologs, and allelic variants of a nucleotide sequence in SEQ ID NO's:1-10 can further be identified by mapping the sequence to the same chromosome or locus as the nucleotide sequence in SEQ ID NO's:1-10.

Also, substantially identical nucleotide sequences may include codons that are altered with respect to the naturally occurring sequence for enhancing expression of a target polypeptide in a particular expression system. For example, the nucleic acid can be one in which one or more codons are altered, and often 10% or more or 20% or more of the codons are altered for optimized expression in bacteria (e.g., E. coli), yeast (e.g., S. cervesiae), human (e.g., 293 cells), insect, or rodent (e.g., hamster) cells.

Methods for Identifying Subjects at Risk of Osteoporosis and Risk of Osteoporosis in a Subject

Methods for prognosing and diagnosing low BMD and its related disorders (e.g., osteoporosis) are included herein. These methods include detecting the presence or absence of one or more polymorphic variations in a nucleotide sequence associated with low BMD, such as variants in or around the loci set forth herein, or a substantially identical sequence thereof, in a sample from a subject, where the presence of a polymorphic variant described herein is indicative of a risk of low BMD or one or more low BMD related disorders (e.g., osteoporosis). Determining a risk of osteoporosis refers to determining whether an individual is at an increased risk of osteoporosis (e.g., intermediate risk or higher risk).

Thus, featured herein is a method for identifying a subject who is at risk of osteoporosis, which comprises detecting low BMD-associated aberration in a nucleic acid sample from the subject. An embodiment is a method for detecting a risk of osteoporosis in a subject, which comprises detecting the presence or absence of a polymorphic variation associated with low BMD at a polymorphic site in a nucleotide sequence in a nucleic acid sample from a subject, where the nucleotide sequence comprises a polynucleotide sequence selected from the group consisting of: (a) a nucleotide sequence of SEQ ID NO's:1-10; (b) a nucleotide sequence which encodes a polypeptide consisting of an amino acid sequence encoded by a nucleotide sequence of SEQ ID NO's:1-10; (c) a nucleotide sequence which encodes a polypeptide that is 90% or more identical to an amino acid sequence encoded by a nucleotide sequence of SEQ ID NO's:1-10, or a nucleotide sequence about 90% or more identical to a nucleotide sequence of SEQ ID NO's:1-10; and (d) a fragment of a nucleotide sequence of (a), (b), or (c) comprising the polymorphic site; whereby the presence of the polymorphic variation is indicative of a predisposition to osteoporosis in the subject. In certain embodiments, polymorphic variants at the positions described herein are detected for determining a risk of osteoporosis, and polymorphic variants at positions in linkage disequilibrium with these positions are detected for determining a risk of osteoporosis. As used herein, “SEQ ID NO's:1-10” refers to individual sequences in SEQ ID NO: 1, 2, 3, 4, 5, 6, 7, 8, 9, 10, 11, and/or 12.

Results from prognostic tests may be combined with other test results to diagnose osteoporosis. For example, prognostic results may be gathered, an initial diagnostic test may be ordered based on a determined predisposition to low BMD, and the results of the analysis may be utilized to diagnose osteoporosis. Also osteoporosis diagnostic methods can be developed from studies used to generate prognostic/diagnostic methods in which populations are stratified into subpopulations having different progressions of osteoporosis. In another embodiment, prognostic results may be gathered; a patient's risk factors for developing osteoporosis analyzed (e.g., age, race, family history, age of menopause); and an initial diagnostic test may be ordered based on a determined predisposition to low BMD.

Risk factors believed to be associated with low BMD include personal history of fracture after age 50; current low bone mass; history of fracture in a 1° relative; being female; being thin and/or having a small frame; low body weight; advanced age; a family history of osteoporosis; estrogen deficiency, especially as a result of menopause which is early or surgically induced; abnormal absence of menstrual periods (amenorrhea); anorexia nervosa; low lifetime calcium intake; use of certain medications, such as corticosteroids and anticonvulsants; low testosterone levels in men; an inactive lifestyle; current cigarette smoking; excessive use of alcohol; being Caucasian or Asian, although African Americans and Hispanic Americans are at significant risk as well. (See National Osteoporosis Foundation; http://www.nof.org/osteoporosis/stats.htm). In an alternative embodiment, the results from predisposition analyses described herein may be combined with other test results indicative of osteoporosis, which were previously, concurrently, or subsequently gathered with respect to the predisposition testing. In these embodiments, the combination of the prognostic test results with other test results can be probative of osteoporosis, and the combination can be utilized as an osteoporosis diagnostic. The results of any test indicative of osteoporosis known in the art may be combined with the methods described herein. Examples of such tests are bone density tests that measure bone density in various sites of the body. Such methods include the measurement of the radiodensity of skeletal radiographs, quantitative computerized tomography, single energy photon absorptiometry, and dual-energy photon absorptiometry. Diagnostic techniques among those useful herein are described in W. A. Peck et al., Physician's Resource Manual on Osteoporosis (1987), published by the National Osteoporosis Foundation (incorporated by reference herein).

Risk of low BMD sometimes is expressed as a probability, such as an odds ratio, percentage, or risk factor. The risk sometimes is expressed as a relative risk with respect to a population average risk of low BMD, and sometimes is expressed as a relative risk with respect to the lowest risk group. Such relative risk assessments often are based upon penetrance values determined by statistical methods and are particularly useful to clinicians and insurance companies for assessing risk of osteoporosis (e.g., a clinician can target appropriate detection, prevention and therapeutic regimens to a patient after determining the patient's risk of osteoporosis, and an insurance company can fine tune actuarial tables based upon population genotype assessments of osteoporosis risk). Risk of osteoporosis sometimes is expressed as an odds ratio, which is the odds of a particular person having a genotype has or will develop osteoporosis with respect to another genotype group (e.g., the most disease protective genotype or population average). The risk often is based upon the presence or absence of one or more polymorphic variants described herein, and also may be based in part upon phenotypic traits of the individual being tested. In an embodiment, two or more polymorphic variations are detected in a CETP, PROL4, GRID2, PDE4D or GPX3/TNIP1 locus. In certain embodiments, 3 or more, or 4, 5, 6, 7, 8, 9, 10, 11, 12, 13, 14, 15, 16, 17, 18, 19 or 20 or more polymorphic variants are detected in the sample. Methods for calculating risk based upon patient data are well known (see, e.g., Agresti, Categorical Data Analysis, 2nd Ed. 2002. Wiley). Allelotyping and genotyping analyses may be carried out in populations other than those exemplified herein to enhance the predictive power of the prognostic method.

The nucleic acid sample typically is isolated from a biological sample obtained from a subject. For example, nucleic acid can be isolated from blood, saliva, sputum, urine, cell scrapings, and biopsy tissue. The nucleic acid sample can be isolated from a biological sample using standard techniques, such as the technique described in Example 2. As used herein, the term “subject” refers primarily to humans but also refers to other mammals such as dogs, cats, and ungulates (e.g., cattle, sheep, and swine). Subjects also include avians (e.g., chickens and turkeys), reptiles, and fish (e.g., salmon), as embodiments described herein can be adapted to nucleic acid samples isolated from any of these organisms. The nucleic acid sample may be isolated from the subject and then directly utilized in a method for determining the presence of a polymorphic variant, or alternatively, the sample may be isolated and then stored (e.g., frozen) for a period of time before being subjected to analysis.

The presence or absence of a polymorphic variant is determined using one or both chromosomal complements represented in the nucleic acid sample. Determining the presence or absence of a polymorphic variant in both chromosomal complements represented in a nucleic acid sample from a subject having a copy of each chromosome is useful for determining the zygosity of an individual for the polymorphic variant (i.e., whether the individual is homozygous or heterozygous for the polymorphic variant). Any oligonucleotide-based diagnostic may be utilized to determine whether a sample includes the presence or absence of a polymorphic variant in a sample. For example, primer extension methods, ligase sequence determination methods (e.g., U.S. Pat. Nos. 5,679,524 and 5,952,174, and WO 01/27326), mismatch sequence determination methods (e.g., U.S. Pat. Nos. 5,851,770; 5,958,692; 6,110,684; and 6,183,958), microarray sequence determination methods, restriction fragment length polymorphism (RFLP), single strand conformation polymorphism detection (SSCP) (e.g., U.S. Pat. Nos. 5,891,625 and 6,013,499), PCR-based assays (e.g., TAQMAN® PCR System (Applied Biosystems)), and nucleotide sequencing methods may be used.

Oligonucleotide extension methods typically involve providing a pair of oligonucleotide primers in a polymerase chain reaction (PCR) or in other nucleic acid amplification methods for the purpose of amplifying a region from the nucleic acid sample that comprises the polymorphic variation. One oligonucleotide primer is complementary to a region 3′ of the polymorphism and the other is complementary to a region 5′ of the polymorphism. A PCR primer pair may be used in methods disclosed in U.S. Pat. Nos. 4,683,195; 4,683,202, 4,965,188; 5,656,493; 5,998,143; 6,140,054; WO 01/27327; and WO 01/27329 for example. PCR primer pairs may also be used in any commercially available machines that perform PCR, such as any of the GENEAMP® Systems available from Applied Biosystems. Also, those of ordinary skill in the art will be able to design oligonucleotide primers based upon a CETP, PROL4, GRID2, PDE4D or GPX3/TNIP1 nucleotide sequence using knowledge available in the art.

Also provided is an extension oligonucleotide that hybridizes to the amplified fragment adjacent to the polymorphic variation. As used herein, the term “adjacent” refers to the 3′ end of the extension oligonucleotide being often 1 nucleotide from the 5′ end of the polymorphic site, and sometimes 2, 3, 4, 5, 6, 7, 8, 9, or 10 nucleotides from the 5′ end of the polymorphic site, in the nucleic acid when the extension oligonucleotide is hybridized to the nucleic acid. The extension oligonucleotide then is extended by one or more nucleotides, and the number and/or type of nucleotides that are added to the extension oligonucleotide determine whether the polymorphic variant is present. Oligonucleotide extension methods are disclosed, for example, in U.S. Pat. Nos. 4,656,127; 4,851,331; 5,679,524; 5,834,189; 5,876,934; 5,908,755; 5,912,118; 5,976,802; 5,981,186; 6,004,744; 6,013,431; 6,017,702; 6,046,005; 6,087,095; 6,210,891; and WO 01/20039. Oligonucleotide extension methods using mass spectrometry are described, for example, in U.S. Pat. Nos. 5,547,835; 5,605,798; 5,691,141; 5,849,542; 5,869,242; 5,928,906; 6,043,031; and 6,194,144, and a method often utilized is described herein in Example 2.

A microarray can be utilized for determining whether a polymorphic variant is present or absent in a nucleic acid sample. A microarray may include any oligonucleotides described herein, and methods for making and using oligonucleotide microarrays suitable for diagnostic use are disclosed in U.S. Pat. Nos. 5,492,806; 5,525,464; 5,589,330; 5,695,940; 5,849,483; 6,018,041; 6,045,996; 6,136,541; 6,142,681; 6,156,501; 6,197,506; 6,223,127; 6,225,625; 6,229,911; 6,239,273; WO 00/52625; WO 01/25485; and WO 01/29259. The microarray typically comprises a solid support and the oligonucleotides may be linked to this solid support by covalent bonds or by non-covalent interactions. The oligonucleotides may also be linked to the solid support directly or by a spacer molecule. A microarray may comprise one or more oligonucleotides complementary to a polymorphic site set forth herein.

A kit also may be utilized for determining whether a polymorphic variant is present or absent in a nucleic acid sample. A kit often comprises one or more pairs of oligonucleotide primers useful for amplifying a fragment of a nucleotide sequence of SEQ ID NO's:1-10 or a substantially identical sequence thereof, where the fragment includes a polymorphic site. The kit sometimes comprises a polymerizing agent, for example, a thermostable nucleic acid polymerase such as one disclosed in U.S. Pat. No. 4,889,818 or 6,077,664. Also, the kit often comprises an elongation oligonucleotide that hybridizes to a CETP, PROL4, GRID2, PDE4D or GPX3/TNIP1 nucleotide sequence in a nucleic acid sample adjacent to the polymorphic site. Where the kit includes an elongation oligonucleotide, it also often comprises chain elongating nucleotides, such as dATP, dTTP, dGTP, dCTP, and dITP, including analogs of dATP, dTTP, dGTP, dCTP and dITP, provided that such analogs are substrates for a thermostable nucleic acid polymerase and can be incorporated into a nucleic acid chain elongated from the extension oligonucleotide. Along with chain elongating nucleotides would be one or more chain terminating nucleotides such as ddATP, ddTTP, ddGTP, ddCTP, and the like. In an embodiment, the kit comprises one or more oligonucleotide primer pairs, a polymerizing agent, chain elongating nucleotides, at least one elongation oligonucleotide, and one or more chain terminating nucleotides. Kits optionally include buffers, vials, microtiter plates, and instructions for use.

An individual identified as being at risk of osteoporosis may be heterozygous or homozygous with respect to the allele associated with low BMD. A subject homozygous for an allele associated with low BMD is at a comparatively high risk of osteoporosis, a subject heterozygous for an allele associated with low BMD is at a comparatively intermediate risk of osteoporosis, and a subject homozygous for an allele associated with normal or high BMD levels (i.e., a decreased risk of low BMD) is at a comparatively low risk of osteoporosis. A genotype may be assessed for a complementary strand, such that the complementary nucleotide at a particular position is detected.

Also featured are methods for determining risk of osteoporosis and/or identifying a subject at risk of osteoporosis by contacting a polypeptide or protein encoded by a CETP, PROL4, GRID2, PDE4D or GPX3/TNIP1 nucleotide sequence from a subject with an antibody that specifically binds to an epitope associated with increased risk of osteoporosis in the polypeptide. In an embodiment, the antibody specifically binds to an epitope comprising an arginine at position 120 in a PROL4 polypeptide.

Applications of Prognostic and Diagnostic Results to Pharmacogenomic Methods

Pharmacogenomics is a discipline that involves tailoring a treatment for a subject according to the subject's genotype as a particular treatment regimen may exert a differential effect depending upon the subject's genotype. For example, based upon the outcome of a prognostic test described herein, a clinician or physician may target pertinent information and preventative or therapeutic treatments to a subject who would be benefited by the information or treatment and avoid directing such information and treatments to a subject who would not be benefited (e.g., the treatment has no therapeutic effect and/or the subject experiences adverse side effects).

The following is an example of a pharmacogenomic embodiment. A particular treatment regimen can exert a differential effect depending upon the subject's genotype. Where a candidate therapeutic exhibits a significant interaction with a major allele and a comparatively weak interaction with a minor allele (e.g., an order of magnitude or greater difference in the interaction), such a therapeutic typically would not be administered to a subject genotyped as being homozygous for the minor allele, and sometimes not administered to a subject genotyped as being heterozygous for the minor allele. In another example, where a candidate therapeutic is not significantly toxic when administered to subjects who are homozygous for a major allele but is comparatively toxic when administered to subjects heterozygous or homozygous for a minor allele, the candidate therapeutic is not typically administered to subjects who are genotyped as being heterozygous or homozygous with respect to the minor allele.

The methods described herein are applicable to pharmacogenomic methods for preventing, alleviating or treating low BMD conditions such as osteoporosis. For example, a nucleic acid sample from an individual may be subjected to a prognostic test described herein. Where one or more polymorphic variations associated with low BMD are identified in a subject, information for preventing or treating osteoporosis and/or one or more osteoporosis treatment regimens then may be prescribed to that subject.

In certain embodiments, a treatment or preventative regimen is specifically prescribed and/or administered to individuals who will most benefit from it based upon their risk of developing osteoporosis assessed by the methods described herein. Thus, provided are methods for identifying a subject predisposed to osteoporosis and then prescribing a therapeutic or preventative regimen to individuals identified as having a predisposition. Thus, certain embodiments are directed to a method for increasing BMD levels or otherwise treating osteoporosis in a subject, which comprises: detecting the presence or absence of a polymorphic variant associated with low BMD in a nucleotide sequence in a nucleic acid sample from a subject, where the nucleotide sequence comprises a polynucleotide sequence selected from the group consisting of: (a) a nucleotide sequence of SEQ ID NO's:1-10; (b) a nucleotide sequence which encodes a polypeptide consisting of an amino acid sequence encoded by a nucleotide sequence of SEQ ID NO's:1-10; (c) a nucleotide sequence which encodes a polypeptide that is 90% or more identical to an amino acid sequence encoded by a nucleotide sequence of SEQ ID NO's:1-10, or a nucleotide sequence about 90% or more identical to a nucleotide sequence of SEQ ID NO's:1-10; and (d) a fragment of a polynucleotide sequence of (a), (b), or (c); and prescribing or administering a treatment regimen to a subject from whom the sample originated where the presence of a polymorphic variation associated with low BMD is detected in the nucleotide sequence. In these methods, predisposition results may be utilized in combination with other test results to diagnose low BMD associated conditions, such as osteoporosis.

Certain preventative treatments often are prescribed to subjects having a predisposition to osteoporosis and where the subject is diagnosed with low BMD or is diagnosed as having symptoms indicative of early stage osteoporosis. Established diagnostic techniques use x-ray and ultrasonography to measure skeletal parameters of bone size, volume and mineral density to predict fracture risk and to assess response to therapy. Such measurements give a “static” value which can be compared to normal values to aid diagnosis of low bone mass and fracture risk (Schott, Cornier et al. 1998). The World Health Organization defines osteoporosis as a bone mineral density level more than 2.5 standard deviations below the young normal mean. The various techniques used to measure bone mineral density include, a) Dual energy X-ray absorptiometry (DXA)—used to measure bone mass at the lumbar spine and hip, but it can also be applied to measuring total skeletal bone mass, soft-tissue composition and other regional bone measurements. DXA is considered the “gold standard” for BMD measurement; b) high-resolution quantitative computed tomography QCT)—highly sensitive, accurate and specific spinal measurements. This technique is more costly, not widely available and involves higher radiation doses than other techniques; c) single-energy x-ray absorptiometry (SXA)—provides accurate radius BMD measurements; d) quantitative ultrasound QUS)—new and promising technique which may have applications in both BMD measurement and assessment or architectural deterioration of bone tissue. Recent studies suggest QUS of calcaneus bone predicts hip fracture as well as DXA (Hans, Dargent-Molina et al. 1996).

An alternative method to predict fracture independently of bone mass is to measure bone turnover. High turnover (bone resorption and formation) is associated with rapid bone loss and is likely to contribute to micro-architectural deterioration (Ross and Knowlton 1998). This is a “dynamic” measurement which is assessed with biochemical markers in urine or serum and can be used effectively in therapy monitoring in preference to BNO measurements. When used in combination with bone mass assessment, biomarkers can provide more accurate fracture predictions over bone mass measurement alone. Osteoporosis-related biomarkers for bone resorption include deoxypyridinoline crosslinks, and osteoporosis-related biomarkers for bone formation include bone alkaline phosphatase and osteocalcin. Some of these biomarkers have been developed for use in diagnostic kits. The current challenge is to reduce the variability of the measurements and improve their reliability and applicability.

The treatment sometimes is preventative (e.g., is prescribed or administered to reduce the probability that a low BMD associated condition arises or progresses), sometimes is therapeutic, and sometimes delays, alleviates or halts the progression of a low BMD associated condition. Any known preventative or therapeutic treatment for alleviating or preventing the occurrence of a low BMD associated disorder is prescribed and/or administered. Preventative treatment for osteoporosis is most effective at the time when bone loss is increasing and before the bones have become fragile and prone to fracturing. Strategies for the prevention of this disease include development of bone density in early adulthood (i.e., building strong bones during childhood and adolescence), and minimization of bone loss in later life. Changes in lifestyle, nutrition and hormonal factors have been shown to affect bone loss. Specifically, a balanced diet rich in calcium and vitamin D, weight-bearing exercise, a healthy lifestyle with no smoking or excessive alcohol intake, and bone density testing and medication when appropriate are known to help reduce the risk of osteoporosis.

As therapeutic approaches for low BMD continue to evolve and improve, the goal of treatments for low BMD related disorders is to intervene even before clinical signs (e.g., impaired glucose tolerance, or IGT) first manifest. Thus, genetic markers associated with susceptibility to low BMD prove useful for early diagnosis, prevention and treatment of low BMD.

As osteoporosis preventative and treatment information can be specifically targeted to subjects in need thereof (e.g., those at risk of low BMD or those that have early stages of osteoporosis), provided herein is a method for preventing or reducing the risk of developing osteoporosis in a subject, which comprises: (a) detecting the presence or absence of a polymorphic variation associated with low BMD at a polymorphic site in a nucleotide sequence in a nucleic acid sample from a subject; (b) identifying a subject with a predisposition to osteoporosis, whereby the presence of the polymorphic variation is indicative of a predisposition to osteoporosis in the subject; and (c) if such a predisposition is identified, providing the subject with information about methods or products to prevent osteoporosis or to delay the onset of osteoporosis. Also provided is a method of targeting information or advertising to a subpopulation of a human population based on the subpopulation being genetically predisposed to a disease or condition, which comprises: (a) detecting the presence or absence of a polymorphic variation associated with low BMD at a polymorphic site in a nucleotide sequence in a nucleic acid sample from a subject; (b) identifying the subpopulation of subjects in which the polymorphic variation is associated with low BMD; and (c) providing information only to the subpopulation of subjects about a particular product which may be obtained and consumed or applied by the subject to help prevent or delay onset of the disease or condition.

Pharmacogenomics methods also may be used to analyze and predict a response to a osteoporosis treatment or a drug. For example, if pharmacogenomics analysis indicates a likelihood that an individual will respond positively to a osteoporosis treatment with a particular drug, the drug may be administered to the individual. Conversely, if the analysis indicates that an individual is likely to respond negatively to treatment with a particular drug, an alternative course of treatment may be prescribed. A negative response may be defined as either the absence of an efficacious response or the presence of toxic side effects. The response to a therapeutic treatment can be predicted in a background study in which subjects in any of the following populations are genotyped: a population that responds favorably to a treatment regimen, a population that does not respond significantly to a treatment regimen, and a population that responds adversely to a treatment regiment (e.g., exhibits one or more side effects). These populations are provided as examples and other populations and subpopulations may be analyzed. Based upon the results of these analyses, a subject is genotyped to predict whether he or she will respond favorably to a treatment regimen, not respond significantly to a treatment regimen, or respond adversely to a treatment regimen.

The tests described herein also are applicable to clinical drug trials. One or more polymorphic variants indicative of response to an agent for treating osteoporosis or to side effects to an agent for treating osteoporosis may be identified using the methods described herein. Thereafter, potential participants in clinical trials of such an agent may be screened to identify those individuals most likely to respond favorably to the drug and exclude those likely to experience side effects. In that way, the effectiveness of drug treatment may be measured in individuals who respond positively to the drug, without lowering the measurement as a result of the inclusion of individuals who are unlikely to respond positively in the study and without risking undesirable safety problems.

Thus, another embodiment is a method of selecting an individual for inclusion in a clinical trial of a treatment or drug comprising the steps of: (a) obtaining a nucleic acid sample from an individual; (b) determining the identity of a polymorphic variation which is associated with a positive response to the treatment or the drug, or at least one polymorphic variation which is associated with a negative response to the treatment or the drug in the nucleic acid sample, and (c) including the individual in the clinical trial if the nucleic acid sample contains said polymorphic variation associated with a positive response to the treatment or the drug or if the nucleic acid sample lacks said polymorphic variation associated with a negative response to the treatment or the drug. In addition, the methods described herein for selecting an individual for inclusion in a clinical trial of a treatment or drug encompass methods with any further limitation described in this disclosure, or those following, specified alone or in any combination. The polymorphic variation may be in a sequence selected individually or in any combination from the group consisting of (i) a nucleotide sequence of SEQ ID NO's:1-10; (ii) a nucleotide sequence which encodes a polypeptide consisting of an amino acid sequence encoded by a nucleotide sequence of SEQ ID NO's:1-10; (iii) a nucleotide sequence which encodes a polypeptide that is 90% or more identical to an amino acid sequence encoded by a nucleotide sequence of SEQ ID NO's:1-10, or a nucleotide sequence about 90% or more identical to a nucleotide sequence of SEQ ID NO's:1-10; and (iv) a fragment of a polynucleotide sequence of (i), (ii), or (iii) comprising the polymorphic site. The including step (c) optionally comprises administering the drug or the treatment to the individual if the nucleic acid sample contains the polymorphic variation associated with a positive response to the treatment or the drug and the nucleic acid sample lacks said biallelic marker associated with a negative response to the treatment or the drug.

Also provided herein is a method of partnering between a diagnostic/prognostic testing provider and a provider of a consumable product, which comprises: (a) the diagnostic/prognostic testing provider detects the presence or absence of a polymorphic variation associated with low BMD at a polymorphic site in a nucleotide sequence in a nucleic acid sample from a subject; (b) the diagnostic/prognostic testing provider identifies the subpopulation of subjects in which the polymorphic variation is associated with low BMD; (c) the diagnostic/prognostic testing provider forwards information to the subpopulation of subjects about a particular product which may be obtained and consumed or applied by the subject to help prevent or delay onset of the disease or condition; and (d) the provider of a consumable product forwards to the diagnostic test provider a fee every time the diagnostic/prognostic test provider forwards information to the subject as set forth in step (c) above.

Compositions Comprising Osteoporosis-Directed Molecules

Featured herein is a composition comprising a cell from a subject having low BMD or at risk of osteoporosis and one or more molecules specifically directed and targeted to a nucleic acid comprising a CETP, PROL4, GRID2, PDE4D or GPX3/TNIP1 nucleotide sequence or amino acid sequence. Such directed molecules include, but are not limited to, a compound that binds to a CETP, PROL4, GRID2, PDE4D or GPX3/TNIP1 nucleotide sequence or amino acid sequence referenced herein; a nucleic acid that hybridizes to a CETP, PROL4, GRID2, PDE4D or GPX3/TNIP1 nucleic acid under conditions of high stringency; a RNAi or siRNA molecule having a strand complementary to a CETP, PROL4, GRID2, PDE4D or GPX3/TNIP1 nucleotide sequence; an antisense nucleic acid complementary to an RNA encoded by a CETP, PROL4, GRID2, PDE4D or GPX3/TNIP1 nucleotide sequence; a ribozyme that hybridizes to a CETP, PROL4, GRID2, PDE4D or GPX3/TNIP1 nucleotide sequence; a nucleic acid aptamer that specifically binds a polypeptide encoded by CETP, PROL4, GRID2, PDE4D or GPX3/TNIP1 nucleotide sequence; and an antibody that specifically binds to a polypeptide encoded by CETP, PROL4, GRID2, PDE4D or GPX3/TNIP1 nucleotide sequence or binds to a nucleic acid having such a nucleotide sequence. In specific embodiments, the osteoporosis directed molecule interacts with a nucleic acid or polypeptide variant associated with osteoporosis, such as variants referenced herein. In other embodiments, the osteoporosis directed molecule interacts with a polypeptide involved in a signal pathway of a polypeptide encoded by a CETP, PROL4, GRID2, PDE4D or GPX3/TNIP1 nucleotide sequence, or a nucleic acid comprising such a nucleotide sequence.

Compositions sometimes include an adjuvant known to stimulate an immune response, and in certain embodiments, an adjuvant that stimulates a T-cell lymphocyte response. Adjuvants are known, including but not limited to an aluminum adjuvant (e.g., aluminum hydroxide); a cytokine adjuvant or adjuvant that stimulates a cytokine response (e.g., interleukin (IL)-12 and/or γ-interferon cytokines); a Freund-type mineral oil adjuvant emulsion (e.g., Freund's complete or incomplete adjuvant); a synthetic lipoid compound; a copolymer adjuvant (e.g., TitreMax); a saponin; Quil A; a liposome; an oil-in-water emulsion (e.g., an emulsion stabilized by Tween 80 and pluronic polyoxyethlene/polyoxypropylene block copolymer (Syntex Adjuvant Formulation); TitreMax; detoxified endotoxin (MPL) and mycobacterial cell wall components (TDW, CWS) in 2% squalene (Ribi Adjuvant System)); a muramyl dipeptide; an immune-stimulating complex (ISCOM, e.g., an Ag-modified saponin/cholesterol micelle that forms stable cage-like structure); an aqueous phase adjuvant that does not have a depot effect (e.g., Gerbu adjuvant); a carbohydrate polymer (e.g., AdjuPrime); L-tyrosine; a manide-oleate compound (e.g., Montanide); an ethylene-vinyl acetate copolymer (e.g., Elvax 40W1,2); or lipid A, for example. Such compositions are useful for generating an immune response against a osteoporosis directed molecule (e.g., an HLA-binding subsequence within a polypeptide encoded by a CETP, PROL4, GRID2, PDE4D or GPX3/TNIP1 nucleotide sequence). In such methods, a peptide having an amino acid subsequence of a polypeptide encoded by a CETP, PROL4, GRID2, PDE4D or GPX3/TNIP1 nucleotide sequence is delivered to a subject, where the subsequence binds to an HLA molecule and induces a CTL lymphocyte response. The peptide sometimes is delivered to the subject as an isolated peptide or as a minigene in a plasmid that encodes the peptide. Methods for identifying HLA-binding subsequences in such polypeptides are known (see e.g., publication WO02/20616 and PCT application US98/01373 for methods of identifying such sequences).

The cell may be in a group of cells cultured in vitro or in a tissue maintained in vitro or present in an animal in vivo (e.g., a rat, mouse, ape or human). In certain embodiments, a composition comprises a component from a cell such as a nucleic acid molecule (e.g., genomic DNA), a protein mixture or isolated protein, for example. The aforementioned compositions have utility in diagnostic, prognostic and pharmacogenomic methods described previously and in osteoporosis therapeutics described hereafter. Certain osteoporosis directed molecules are described in greater detail below.

Compounds

Compounds can be obtained using any of the numerous approaches in combinatorial library methods known in the art, including: biological libraries; peptoid libraries (libraries of molecules having the functionalities of peptides, but with a novel, non-peptide backbone which are resistant to enzymatic degradation but which nevertheless remain bioactive (see, e.g., Zuckermann et al., J. Med. Chem. 37: 2678-85 (1994)); spatially addressable parallel solid phase or solution phase libraries; synthetic library methods requiring deconvolution; “one-bead one-compound” library methods; and synthetic library methods using affinity chromatography selection. Biological library and peptoid library approaches are typically limited to peptide libraries, while the other approaches are applicable to peptide, non-peptide oligomer or small molecule libraries of compounds (Lam, Anticancer Drug Des. 12: 145, (1997)). Examples of methods for synthesizing molecular libraries are described, for example, in DeWitt et al., Proc. Natl. Acad. Sci. U.S.A. 90: 6909 (1993); Erb et al., Proc. Natl. Acad. Sci. USA 91: 11422 (1994); Zuckermann et al., J. Med. Chem. 37: 2678 (1994); Cho et al., Science 261: 1303 (1993); Carrell et al., Angew. Chem. Int. Ed. Engl. 33: 2059 (1994); Carell et al., Angew. Chem. Int. Ed. Engl. 33: 2061 (1994); and in Gallop et al., J. Med. Chem. 37: 1233 (1994).

Libraries of compounds may be presented in solution (e.g., Houghten, Biotechniques 13: 412-421 (1992)), or on beads (Lam, Nature 354: 82-84 (1991)), chips (Fodor, Nature 364: 555-556 (1993)), bacteria or spores (Ladner, U.S. Pat. No. 5,223,409), plasmids (Cull et al., Proc. Natl. Acad. Sci. USA 89: 1865-1869 (1992)) or on phage (Scott and Smith, Science 249: 386-390 (1990); Devlin, Science 249: 404-406 (1990); Cwirla et al., Proc. Natl. Acad. Sci. 87: 6378-6382 (1990); Felici, J. Mol. Biol. 222: 301-310 (1991); Ladner supra.).

A compound sometimes alters expression and sometimes alters activity of a polypeptide target and may be a small molecule. Small molecules include, but are not limited to, peptides, peptidomimetics (e.g., peptoids), amino acids, amino acid analogs, polynucleotides, polynucleotide analogs, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

Antisense Nucleic Acid Molecules, Ribozymes, RNAi, siRNA and Modified Nucleic Acid Molecules

An “antisense” nucleic acid refers to a nucleotide sequence complementary to a “sense” nucleic acid encoding a polypeptide, e.g., complementary to the coding strand of a double-stranded cDNA molecule or complementary to an mRNA sequence. The antisense nucleic acid can be complementary to an entire coding strand (e.g., SEQ ID NO: 7-13), or to a portion thereof or a substantially identical sequence thereof. In another embodiment, the antisense nucleic acid molecule is antisense to a “noncoding region” of the coding strand of a nucleotide sequence (e.g., 5′ and 3′ untranslated regions in SEQ ID NO's:1-5).

An antisense nucleic acid can be designed such that it is complementary to the entire coding region of an mRNA encoded by a nucleotide sequence (e.g., SEQ ID NO's:1-10), and often the antisense nucleic acid is an oligonucleotide antisense to only a portion of a coding or noncoding region of the mRNA. For example, the antisense oligonucleotide can be complementary to the region surrounding the translation start site of the mRNA, e.g., between the −10 and +10 regions of the target gene nucleotide sequence of interest. An antisense oligonucleotide can be, for example, about 7, 10, 15, 20, 25, 30, 35, 40, 45, 50, 55, 60, 65, 70, 75, 80, or more nucleotides in length. The antisense nucleic acids, which include the ribozymes described hereafter, can be designed to target a CETP, PROL4, GRID2, PDE4D or GPX3/TNIP1 nucleotide sequence, often a variant associated with low BMD, or a substantially identical sequence thereof. Among the variants, minor alleles and major alleles can be targeted, and those associated with a higher risk of osteoporosis are often designed, tested, and administered to subjects.

An antisense nucleic acid can be constructed using chemical synthesis and enzymatic ligation reactions using standard procedures. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Antisense nucleic acid also can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

When utilized as therapeutics, antisense nucleic acids typically are administered to a subject (e.g., by direct injection at a tissue site) or generated in situ such that they hybridize with or bind to cellular mRNA and/or genomic DNA encoding a polypeptide and thereby inhibit expression of the polypeptide, for example, by inhibiting transcription and/or translation. Alternatively, antisense nucleic acid molecules can be modified to target selected cells and then are administered systemically. For systemic administration, antisense molecules can be modified such that they specifically bind to receptors or antigens expressed on a selected cell surface, for example, by linking antisense nucleic acid molecules to peptides or antibodies which bind to cell surface receptors or antigens. Antisense nucleic acid molecules can also be delivered to cells using the vectors described herein. Sufficient intracellular concentrations of antisense molecules are achieved by incorporating a strong promoter, such as a pol II or pol III promoter, in the vector construct.

Antisense nucleic acid molecules sometimes are *-anomeric nucleic acid molecules. An *-anomeric nucleic acid molecule forms specific double-stranded hybrids with complementary RNA in which, contrary to the usual *-units, the strands run parallel to each other (Gaultier et al., Nucleic Acids. Res. 15: 6625-6641 (1987)). Antisense nucleic acid molecules can also comprise a 2′-o-methylribonucleotide (Inoue et al., Nucleic Acids Res. 15: 6131-6148 (1987)) or a chimeric RNA-DNA analogue (Inoue et al., FEBS Lett. 215: 327-330 (1987)). Antisense nucleic acids sometimes are composed of DNA or PNA or any other nucleic acid derivatives described previously.

In another embodiment, an antisense nucleic acid is a ribozyme. A ribozyme having specificity for a CETP, PROL4, GRID2, PDE4D or GPX3/TNIP1 nucleotide sequence can include one or more sequences complementary to such a nucleotide sequence, and a sequence having a known catalytic region responsible for mRNA cleavage (see e.g., U.S. Pat. No. 5,093,246 or Haselhoff and Gerlach, Nature 334: 585-591 (1988)). For example, a derivative of a Tetrahymena L-19 IVS RNA is sometimes utilized in which the nucleotide sequence of the active site is complementary to the nucleotide sequence to be cleaved in a mRNA (see e.g., Cech et al. U.S. Pat. No. 4,987,071; and Cech et al. U.S. Pat. No. 5,116,742). Also, target mRNA sequences can be used to select a catalytic RNA having a specific ribonuclease activity from a pool of RNA molecules (see e.g., Bartel & Szostak, Science 261: 1411-1418(1993)).

Osteoporosis directed molecules include in certain embodiments nucleic acids that can form triple helix structures with a CETP, PROL4, GRID2, PDE4D or GPX3/TNIP1 nucleotide sequence or a substantially identical sequence thereof, especially one that includes a regulatory region that controls expression of a polypeptide. Gene expression can be inhibited by targeting nucleotide sequences complementary to the regulatory region of a nucleotide sequence referenced herein or a substantially identical sequence (e.g., promoter and/or enhancers) to form triple helical structures that prevent transcription of a gene in target cells (see e.g., Helene, Anticancer Drug Des. 6(6): 569-84 (1991); Helene et al., Ann. N.Y. Acad. Sci. 660: 27-36 (1992); and Maher, Bioassays 14(12): 807-15 (1992). Potential sequences that can be targeted for triple helix formation can be increased by creating a so-called “switchback” nucleic acid molecule. Switchback molecules are synthesized in an alternating 5′-3′, 3′-5′ manner, such that they base pair with first one strand of a duplex and then the other, eliminating the necessity for a sizeable stretch of either purines or pyrimidines to be present on one strand of a duplex.

Osteoporosis directed molecules include RNAi and siRNA nucleic acids. Gene expression may be inhibited by the introduction of double-stranded RNA (dsRNA), which induces potent and specific gene silencing, a phenomenon called RNA interference or RNAi. See, e.g., Fire et al., U.S. Pat. No. 6,506,559; Tuschl et al. PCT International Publication No. WO 01/75164; Kay et al. PCT International Publication No. WO 03/010180A1; or Bosher J M, Labouesse, Nat Cell Biol 2000 February; 2(2):E31-6. This process has been improved by decreasing the size of the double-stranded RNA to 20-24 base pairs (to create small-interfering RNAs or siRNAs) that “switched off” genes in mammalian cells without initiating an acute phase response, i.e., a host defense mechanism that often results in cell death (see, e.g., Caplen et al. Proc Natl Acad Sci USA. 2001 Aug. 14; 98(17):9742-7 and Elbashir et al. Methods 2002 February; 26(2): 199-213). There is increasing evidence of post-transcriptional gene silencing by RNA interference (RNAi) for inhibiting targeted expression in mammalian cells at the mRNA level, in human cells. There is additional evidence of effective methods for inhibiting the proliferation and migration of tumor cells in human patients, and for inhibiting metastatic cancer development (see, e.g., U.S. Patent Application No. US2001000993183; Caplen et al. Proc Natl Acad Sci USA; and Abdeffahmani et al. Mol Cell Biol 2001 November 21(21):7256-67).

An “siRNA” or “RNAi” refers to a nucleic acid that forms a double stranded RNA and has the ability to reduce or inhibit expression of a gene or target gene when the siRNA is delivered to or expressed in the same cell as the gene or target gene. “siRNA” refers to short double-stranded RNA formed by the complementary strands. Complementary portions of the siRNA that hybridize to form the double stranded molecule often have substantial or complete identity to the target molecule sequence. In one embodiment, an siRNA refers to a nucleic acid that has substantial or complete identity to a target gene and forms a double stranded siRNA.

When designing the siRNA molecules, the targeted region often is selected from a given DNA sequence beginning 50 to 100 nucleotides downstream of the start codon. See, e.g., Elbashir et al., Methods 26:199-213 (2002). Initially, 5′ or 3′ UTRs and regions nearby the start codon were avoided assuming that UTR-binding proteins and/or translation initiation complexes may interfere with binding of the siRNP or RISC endonuclease complex. Sometimes regions of the target 23 nucleotides in length conforming to the sequence motif AA(N19)TT (N, an nucleotide), and regions with approximately 30% to 70% G/C-content (often about 50% G/C-content) often are selected. If no suitable sequences are found, the search often is extended using the motif NA(N21). The sequence of the sense siRNA sometimes corresponds to (N19) TT or N21 (position 3 to 23 of the 23-nt motif), respectively. In the latter case, the 3′ end of the sense siRNA often is converted to TT. The rationale for this sequence conversion is to generate a symmetric duplex with respect to the sequence composition of the sense and antisense 3′ overhangs. The antisense siRNA is synthesized as the complement to position 1 to 21 of the 23-nt motif. Because position 1 of the 23-nt motif is not recognized sequence-specifically by the antisense siRNA, the 3′-most nucleotide residue of the antisense siRNA can be chosen deliberately. However, the penultimate nucleotide of the antisense siRNA (complementary to position 2 of the 23-nt motif) often is complementary to the targeted sequence. For simplifying chemical synthesis, TT often is utilized. siRNAs corresponding to the target motif NAR(N17)YNN, where R is purine (A,G) and Y is pyrimidine (C,U), often are selected. Respective 21 nucleotide sense and antisense siRNAs often begin with a purine nucleotide and can also be expressed from pol III expression vectors without a change in targeting site. Expression of RNAs from pol III promoters often is efficient when the first transcribed nucleotide is a purine.

The sequence of the siRNA can correspond to the full length target gene, or a subsequence thereof. Often, the siRNA is about 15 to about 50 nucleotides in length (e.g., each complementary sequence of the double stranded siRNA is 15-50 nucleotides in length, and the double stranded siRNA is about 15-50 base pairs in length, sometimes about 20-30 nucleotides in length or about 20-25 nucleotides in length, e.g., 20, 21, 22, 23, 24, 25, 26, 27, 28, 29, or 30 nucleotides in length. The siRNA sometimes is about 21 nucleotides in length. Methods of using siRNA are well known in the art, and specific siRNA molecules may be purchased from a number of companies including Dharmacon Research, Inc. An siRNA molecule sometimes is of a different chemical composition as compared to native RNA that imparts increased stability in cells (e.g., decreased susceptibility to degradation), and sometimes includes one or more modifications in siSTABLE RNA described at the http address www.dharmacon.com.

Antisense, ribozyme, RNAi and siRNA nucleic acids can be altered to form modified nucleic acid molecules. The nucleic acids can be altered at base moieties, sugar moieties or phosphate backbone moieties to improve stability, hybridization, or solubility of the molecule. For example, the deoxyribose phosphate backbone of nucleic acid molecules can be modified to generate peptide nucleic acids (see Hyrup et al., Bioorganic & Medicinal Chemistry 4 (1): 5-23 (1996)). As used herein, the terms “peptide nucleic acid” or “PNA” refers to a nucleic acid mimic such as a DNA mimic, in which the deoxyribose phosphate backbone is replaced by a pseudopeptide backbone and only the four natural nucleobases are retained. The neutral backbone of a PNA can allow for specific hybridization to DNA and RNA under conditions of low ionic strength. Synthesis of PNA oligomers can be performed using standard solid phase peptide synthesis protocols as described, for example, in Hyrup et al., (1996) supra and Perry-O'Keefe et al., Proc. Natl. Acad. Sci. 93: 14670-675 (1996).

PNA nucleic acids can be used in prognostic, diagnostic, and therapeutic applications. For example, PNAs can be used as antisense or antigene agents for sequence-specific modulation of gene expression by, for example, inducing transcription or translation arrest or inhibiting replication. PNA nucleic acid molecules can also be used in the analysis of single base pair mutations in a gene, (e.g., by PNA-directed PCR clamping); as “artificial restriction enzymes” when used in combination with other enzymes, (e.g., S1 nucleases (Hyrup (1996) supra)); or as probes or primers for DNA sequencing or hybridization (Hyrup et al., (1996) supra; Perry-O'Keefe supra).

In other embodiments, oligonucleotides may include other appended groups such as peptides (e.g., for targeting host cell receptors in vivo), or agents facilitating transport across cell membranes (see e.g., Letsinger et al., Proc. Natl. Acad. Sci. USA 86: 6553-6556 (1989); Lemaitre et al., Proc. Natl. Acad. Sci. USA 84: 648-652 (1987); PCT Publication No. WO88/09810) or the blood-brain barrier (see, e.g., PCT Publication No. WO 89/10134). In addition, oligonucleotides can be modified with hybridization-triggered cleavage agents (See, e.g., Krol et al., Bio-Techniques 6: 958-976 (1988)) or intercalating agents. (See, e.g., Zon, Pharm. Res. 5: 539-549 (1988)). To this end, the oligonucleotide may be conjugated to another molecule, (e.g., a peptide, hybridization triggered cross-linking agent, transport agent, or hybridization-triggered cleavage agent).

Also included herein are molecular beacon oligonucleotide primer and probe molecules having one or more regions complementary to a CETP, PROL4, GRID2, PDE4D or GPX3/TNIP1 nucleotide sequence or a substantially identical sequence thereof, two complementary regions one having a fluorophore and one a quencher such that the molecular beacon is useful for quantifying the presence of the nucleic acid in a sample. Molecular beacon nucleic acids are described, for example, in Lizardi et al., U.S. Pat. No. 5,854,033; Nazarenko et al., U.S. Pat. No. 5,866,336, and Livak et al., U.S. Pat. No. 5,876,930.

Antibody

The term “antibody” as used herein refers to an immunoglobulin molecule or immunologically active portion thereof, i.e., an antigen-binding portion. Examples of immunologically active portions of immunoglobulin molecules include F(ab) and F(ab′)₂ fragments which can be generated by treating the antibody with an enzyme such as pepsin. An antibody sometimes is a polyclonal, monoclonal, recombinant (e.g., a chimeric or humanized), fully human, non-human (e.g., murine), or a single chain antibody. An antibody may have effector function and can fix complement, and is sometimes coupled to a toxin or imaging agent.

Antibodies

A full-length polypeptide or antigenic peptide fragment encoded by a nucleotide sequence referenced herein can be used as an immunogen or can be used to identify antibodies made with other immunogens, e.g., cells, membrane preparations, and the like. An antigenic peptide often includes at least 8 amino acid residues of the amino acid sequences encoded by a nucleotide sequence referenced herein, or substantially identical sequence thereof, and encompasses an epitope. Antigenic peptides sometimes include 10 or more amino acids, 15 or more amino acids, 20 or more amino acids, or 30 or more amino acids. Hydrophilic and hydrophobic fragments of polypeptides sometimes are used as immunogens.

Epitopes encompassed by the antigenic peptide are regions located on the surface of the polypeptide (e.g., hydrophilic regions) as well as regions with high antigenicity. For example, an Emini surface probability analysis of the human polypeptide sequence can be used to indicate the regions that have a particularly high probability of being localized to the surface of the polypeptide and are thus likely to constitute surface residues useful for targeting antibody production. The antibody may bind an epitope on any domain or region on polypeptides described herein.

Also, chimeric, humanized, and completely human antibodies are useful for applications which include repeated administration to subjects. Chimeric and humanized monoclonal antibodies, comprising both human and non-human portions, can be made using standard recombinant DNA techniques. Such chimeric and humanized monoclonal antibodies can be produced by recombinant DNA techniques known in the art, for example using methods described in Robinson et al International Application No. PCT/US86/02269; Akira, et al European Patent Application 184,187; Taniguchi, M., European Patent Application 171,496; Morrison et al European Patent Application 173,494; Neuberger et al PCT International Publication No. WO 86/01533; Cabilly et al U.S. Pat. No. 4,816,567; Cabilly et al European Patent Application 125,023; Better et al., Science 240: 1041-1043 (1988); Liu et al., Proc. Natl. Acad. Sci. USA 84: 3439-3443 (1987); Liu et al., J. Immunol. 139: 3521-3526 (1987); Sun et al., Proc. Natl. Acad. Sci. USA 84: 214-218 (1987); Nishimura et al., Canc. Res. 47: 999-1005 (1987); Wood et al., Nature 314: 446-449 (1985); and Shaw et al., J. Natl. Cancer Inst. 80: 1553-1559 (1988); Morrison, S. L., Science 229:1202-1207(1985); Oi et al., BioTechniques 4: 214(1986); Winter U.S. Pat. No. 5,225,539; Jones et al., Nature 321: 552-525 (1986); Verhoeyan et al., Science 239: 1534; and Beidler et al., J. Immunol. 141: 4053-4060 (1988).

Completely human antibodies are particularly desirable for therapeutic treatment of human patients. Such antibodies can be produced using transgenic mice that are incapable of expressing endogenous immunoglobulin heavy and light chains genes, but which can express human heavy and light chain genes. See, for example, Lonberg and Huszar, Int. Rev. Immunol. 13: 65-93 (1995); and U.S. Pat. Nos. 5,625,126; 5,633,425; 5,569,825; 5,661,016; and 5,545,806. In addition, companies such as Abgenix, Inc. (Fremont, Calif.) and Medarex, Inc. (Princeton, N.J.), can be engaged to provide human antibodies directed against a selected antigen using technology similar to that described above. Completely human antibodies that recognize a selected epitope also can be generated using a technique referred to as “guided selection.” In this approach a selected non-human monoclonal antibody (e.g., a murine antibody) is used to guide the selection of a completely human antibody recognizing the same epitope. This technology is described for example by Jespers et al., Bio/Technology 12: 899-903 (1994).

An antibody can be a single chain antibody. A single chain antibody (scFV) can be engineered (see, e.g., Colcher et al., Ann. N Y Acad. Sci. 880: 263-80 (1999); and Reiter, Clin. Cancer Res. 2: 245-52 (1996)). Single chain antibodies can be dimerized or multimerized to generate multivalent antibodies having specificities for different epitopes of the same target polypeptide.

Antibodies also may be selected or modified so that they exhibit reduced or no ability to bind an Fc receptor. For example, an antibody may be an isotype or subtype, fragment or other mutant, which does not support binding to an Fc receptor (e.g., it has a mutagenized or deleted Fc receptor binding region).

Also, an antibody (or fragment thereof) may be conjugated to a therapeutic moiety such as a cytotoxin, a therapeutic agent or a radioactive metal ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, l dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thiotepa chlorambucil, melphalan, carmustine (BCNU) and lomustine (CCNU), cyclophosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

Antibody conjugates can be used for modifying a given biological response. For example, the drug moiety may be a protein or polypeptide possessing a desired biological activity. Such proteins may include, for example, a toxin such as abrin, ricin A, pseudomonas exotoxin, or diphtheria toxin; a polypeptide such as tumor necrosis factor, γ-interferon, α-interferon, nerve growth factor, platelet derived growth factor, tissue plasminogen activator; or, biological response modifiers such as, for example, lymphokines, interleukin-1 (“IL-1”), interleukin-2 (“IL-2”), interleukin-6 (“IL-6”), granulocyte macrophage colony stimulating factor (“GM-CSF”), granulocyte colony stimulating factor “G-CSF”), or other growth factors. Also, an antibody can be conjugated to a second antibody to form an antibody heteroconjugate as described by Segal in U.S. Pat. No. 4,676,980, for example.

An antibody (e.g., monoclonal antibody) can be used to isolate target polypeptides by standard techniques, such as affinity chromatography or immunoprecipitation. Moreover, an antibody can be used to detect a target polypeptide (e.g., in a cellular lysate or cell supernatant) in order to evaluate the abundance and pattern of expression of the polypeptide. Antibodies can be used diagnostically to monitor polypeptide levels in tissue as part of a clinical testing procedure, e.g., to determine the efficacy of a given treatment regimen. Detection can be facilitated by coupling (i.e., physically linking) the antibody to a detectable substance (i.e., antibody labeling). Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H. Also, an antibody can be utilized as a test molecule for determining whether it can treat osteoporosis, and as a therapeutic for administration to a subject for treating osteoporosis.

An antibody can be made by immunizing with a purified antigen, or a fragment thereof, e.g., a fragment described herein, a membrane associated antigen, tissues, e.g., crude tissue preparations, whole cells, preferably living cells, lysed cells, or cell fractions.

Included herein are antibodies which bind only a native polypeptide, only denatured or otherwise non-native polypeptide, or which bind both, as well as those having linear or conformational epitopes. Conformational epitopes sometimes can be identified by selecting antibodies that bind to native but not denatured polypeptide. Also featured are antibodies that specifically bind to a polypeptide variant associated with low BMD.

Methods for Identifying Candidate Therapeutics for Treating Osteoporosis

Current treatment for osteoporosis can help stop further bone loss and fractures, but there is still a clear need for specific anabolic agents that considerably increase bone formation in people who have already suffered substantial bone loss. There are no such drugs currently approved. Known osteoporosis treatments include bone-active phosphonates (See, e.g., U.S. Pat. No. 6,329,354), bisphosphonates, e.g., alendronate (Fosamax®) and risedronate (Actonel®); calcitonin (Niacalcin®); estrogen and hormone therapy, e.g., estrogens (Climara®, Estrace®, Estraderm®, Estratab®, Ogen®, Ortho-Est®, Vivelle®, Premarin®, and others) and estrogens and progestins (brand names, such as Activella™, FemHrt®, Premphase®, Prempro®, and others); parathyroid hormone, e.g., teriparatide {PTH 1-34} (Forteo®); as well as, estrogen and estrogen receptor modulators (SERMs), e.g., raloxifene HCl (Evista®), sodium fluoride and vitamin D metabolites. Any of the above therapeutics may be administered alone or in combination (e.g., bone-active phosphonates and estrogen and hormone therapy). Current therapeutic approaches were largely developed in the absence of defined molecular targets or even a solid understanding of disease pathogenesis. Therefore, provided are methods of identifying candidate therapeutics that target biochemical pathways related to the development of osteoporosis.

Thus, featured herein are methods for identifying a candidate therapeutic for treating osteoporosis. The methods comprise contacting a test molecule with a target molecule in a system. A “target molecule” as used herein refers to a CETP, PROL4, GRID2, PDE4D or GPX3/TNIP1 nucleic acid, a substantially identical nucleic acid thereof, or a fragment thereof, and an encoded polypeptide of the foregoing. The methods also comprise determining the presence or absence of an interaction between the test molecule and the target molecule, where the presence of an interaction between the test molecule and the nucleic acid or polypeptide identifies the test molecule as a candidate osteoporosis therapeutic. The interaction between the test molecule and the target molecule may be quantified.

Test molecules and candidate therapeutics include, but are not limited to, compounds, antisense nucleic acids, siRNA molecules, ribozymes, polypeptides or proteins encoded by a CETP, PROL4, GRID2, PDE4D or GPX3/TNIP1 nucleotide sequence, or a substantially identical sequence or fragment thereof, and immunotherapeutics (e.g., antibodies and HLA-presented polypeptide fragments). A test molecule or candidate therapeutic may act as a modulator of target molecule concentration or target molecule function in a system. A “modulator” may agonize (i.e., up-regulates) or antagonize (i.e., down-regulates) a target molecule concentration partially or completely in a system by affecting such cellular functions as DNA replication and/or DNA processing (e.g. DNA methylation or DNA repair), RNA transcription and/or RNA processing (e.g., removal of intronic sequences and/or translocation of spliced mRNA from the nucleus), polypeptide production (e.g., translation of the polypeptide from mRNA), and/or polypeptide post-translational modification (e.g., glycosylation, phosphorylation, and proteolysis of pro-polypeptides). A modulator may also agonize or antagonize a biological function of a target molecule partially or completely, where the function may include adopting a certain structural conformation, interacting with one or more binding partners, ligand binding, catalysis (e.g., phosphorylation, dephosphorylation, hydrolysis, methylation, and isomerization), and an effect upon a cellular event (e.g., effecting progression of osteoporosis). In certain embodiments, a candidate therapeutic increases BMD.

As used herein, the term “system” refers to a cell free in vitro environment and a cell-based environment such as a collection of cells, a tissue, an organ, or an organism. A system is “contacted” with a test molecule in a variety of manners, including adding molecules in solution and allowing them to interact with one another by diffusion, cell injection, and any administration routes in an animal. As used herein, the term “interaction” refers to an effect of a test molecule on test molecule, where the effect sometimes is binding between the test molecule and the target molecule, and sometimes is an observable change in cells, tissue, or organism.

There are many standard methods for detecting the presence or absence of interaction between a test molecule and a target molecule. For example, titrametric, acidimetric, radiometric, NMR, monolayer, polarographic, spectrophotometric, fluorescent, and ESR assays probative of a target molecule interaction may be utilized.

Test molecule/target molecule interactions can be detected and/or quantified using assays known in the art. For example, an interaction can be determined by labeling the test molecule and/or the target molecule, where the label is covalently or non-covalently attached to the test molecule or target molecule. The label is sometimes a radioactive molecule such as ¹²⁵I, ¹³¹I, ³⁵S or ³H, which can be detected by direct counting of radioemission or by scintillation counting. Also, enzymatic labels such as horseradish peroxidase, alkaline phosphatase, or luciferase may be utilized where the enzymatic label can be detected by determining conversion of an appropriate substrate to product. In addition, presence or absence of an interaction can be determined without labeling. For example, a microphysiometer (e.g., Cytosensor) is an analytical instrument that measures the rate at which a cell acidifies its environment using a light-addressable potentiometric sensor (LAPS). Changes in this acidification rate can be used as an indication of an interaction between a test molecule and target molecule (McConnell, H. M. et al., Science 257: 1906-1912 (1992)).

In cell-based systems, cells typically include a CETP, PROL4, GRID2, PDE4D or GPX3/TNIP1 nucleic acid, an encoded polypeptide, or substantially identical nucleic acid or polypeptide thereof, and are often of mammalian origin, although the cell can be of any origin. Whole cells, cell homogenates, and cell fractions (e.g., cell membrane fractions) can be subjected to analysis. Where interactions between a test molecule with a target polypeptide are monitored, soluble and/or membrane bound forms of the polypeptide may be utilized. Where membrane-bound forms of the polypeptide are used, it may be desirable to utilize a solubilizing agent. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, 3-[(3-cholamidopropyl)dimethylamminio]-1-propane sulfonate (CHAPS), 3-[(3-cholamidopropyl)dimethylamminio]-2-hydroxy-1-propane sulfonate (CHAPSO), or N-dodecyl-N,N-dimethyl-3-ammonio-1-propane sulfonate.

An interaction between a test molecule and target molecule also can be detected by monitoring fluorescence energy transfer (FET) (see, e.g., Lakowicz et al., U.S. Pat. No. 5,631,169; Stavrianopoulos et al. U.S. Pat. No. 4,868,103). A fluorophore label on a first, “donor” molecule is selected such that its emitted fluorescent energy will be absorbed by a fluorescent label on a second, “acceptor” molecule, which in turn is able to fluoresce due to the absorbed energy. Alternately, the “donor” polypeptide molecule may simply utilize the natural fluorescent energy of tryptophan residues. Labels are chosen that emit different wavelengths of light, such that the “acceptor” molecule label may be differentiated from that of the “donor”. Since the efficiency of energy transfer between the labels is related to the distance separating the molecules, the spatial relationship between the molecules can be assessed. In a situation in which binding occurs between the molecules, the fluorescent emission of the “acceptor” molecule label in the assay should be maximal. An FET binding event can be conveniently measured through standard fluorometric detection means well known in the art (e.g. using a fluorimeter).

In another embodiment, determining the presence or absence of an interaction between a test molecule and a target molecule can be effected by monitoring surface plasmon resonance (see, e.g., Sjolander & Urbaniczk, Anal. Chem. 63: 2338-2345 (1991) and Szabo et al., Curr. Opin. Struct. Biol. 5: 699-705 (1995)). “Surface plasmon resonance” or “biomolecular interaction analysis (BIA)” can be utilized to detect biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal which can be used as an indication of real-time reactions between biological molecules.

In another embodiment, the target molecule or test molecules are anchored to a solid phase, facilitating the detection of target molecule/test molecule complexes and separation of the complexes from free, uncomplexed molecules. The target molecule or test molecule is immobilized to the solid support. In an embodiment, the target molecule is anchored to a solid surface, and the test molecule, which is not anchored, can be labeled, either directly or indirectly, with detectable labels discussed herein.

It may be desirable to immobilize a target molecule, an anti-target molecule antibody, and/or test molecules to facilitate separation of target molecule/test molecule complexes from uncomplexed forms, as well as to accommodate automation of the assay. The attachment between a test molecule and/or target molecule and the solid support may be covalent or non-covalent (see, e.g., U.S. Pat. No. 6,022,688 for non-covalent attachments). The solid support may be one or more surfaces of the system, such as one or more surfaces in each well of a microtiter plate, a surface of a silicon wafer, a surface of a bead (see, e.g., Lam, Nature 354: 82-84 (1991)) that is optionally linked to another solid support, or a channel in a microfluidic device, for example. Types of solid supports, linker molecules for covalent and non-covalent attachments to solid supports, and methods for immobilizing nucleic acids and other molecules to solid supports are well known (see, e.g., U.S. Pat. Nos. 6,261,776; 5,900,481; 6,133,436; and 6,022,688; and WIPO publication WO 01/18234).

In an embodiment, target molecule may be immobilized to surfaces via biotin and streptavidin. For example, biotinylated target polypeptide can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical). In another embodiment, a target polypeptide can be prepared as a fusion polypeptide. For example, glutathione-S-transferase/target polypeptide fusion can be adsorbed onto glutathione sepharose beads (Sigma Chemical, St. Louis, Mo.) or glutathione derivitized microtiter plates, which are then combined with a test molecule under conditions conducive to complex formation (e.g., at physiological conditions for salt and pH). Following incubation, the beads or microtiter plate wells are washed to remove any unbound components, or the matrix is immobilized in the case of beads, and complex formation is determined directly or indirectly as described above. Alternatively, the complexes can be dissociated from the matrix, and the level of target molecule binding or activity is determined using standard techniques.

In an embodiment, the non-immobilized component is added to the coated surface containing the anchored component. After the reaction is complete, unreacted components are removed (e.g., by washing) under conditions such that a significant percentage of complexes formed will remain immobilized to the solid surface. The detection of complexes anchored on the solid surface can be accomplished in a number of manners. Where the previously non-immobilized component is pre-labeled, the detection of label immobilized on the surface indicates that complexes were formed. Where the previously non-immobilized component is not pre-labeled, an indirect label can be used to detect complexes anchored on the surface, e.g., by adding a labeled antibody specific for the immobilized component, where the antibody, in turn, can be directly labeled or indirectly labeled with, e.g., a labeled anti-Ig antibody.

In another embodiment, an assay is performed utilizing antibodies that specifically bind target molecule or test molecule but do not interfere with binding of the target molecule to the test molecule. Such antibodies can be derivitized to a solid support, and unbound target molecule may be immobilized by antibody conjugation. Methods for detecting such complexes, in addition to those described above for the GST-immobilized complexes, include immunodetection of complexes using antibodies reactive with the target molecule, as well as enzyme-linked assays which rely on detecting an enzymatic activity associated with the target molecule.

Cell free assays also can be conducted in a liquid phase. In such an assay, reaction products are separated from unreacted components, by any of a number of standard techniques, including but not limited to: differential centrifugation (see, e.g., Rivas, G., and Minton, Trends Biochem Sci August; 18(8): 284-7 (1993)); chromatography (gel filtration chromatography, ion-exchange chromatography); electrophoresis (see, e.g., Ausubel et al., eds. Current Protocols in Molecular Biology, J. Wiley: New York (1999)); and immunoprecipitation (see, e.g., Ausubel et al., eds., supra). Media and chromatographic techniques are known to one skilled in the art (see, e.g., Heegaard, J Mol. Recognit. Winter; 11(1-6): 141-8 (1998); Hage & Tweed, J. Chromatogr. B Biomed. Sci. Appl. October 10; 699 (1-2): 499-525 (1997)). Further, fluorescence energy transfer may also be conveniently utilized, as described herein, to detect binding without further purification of the complex from solution.

In another embodiment, modulators of target molecule expression are identified. For example, a cell or cell free mixture is contacted with a candidate compound and the expression of target mRNA or target polypeptide is evaluated relative to the level of expression of target mRNA or target polypeptide in the absence of the candidate compound. When expression of target mRNA or target polypeptide is greater in the presence of the candidate compound than in its absence, the candidate compound is identified as an agonist of target mRNA or target polypeptide expression. Alternatively, when expression of target mRNA or target polypeptide is less (e.g., less with statistical significance) in the presence of the candidate compound than in its absence, the candidate compound is identified as an antagonist or inhibitor of target mRNA or target polypeptide expression. The level of target mRNA or target polypeptide expression can be determined by methods described herein.

In another embodiment, binding partners that interact with a target molecule are detected. The target molecules can interact with one or more cellular or extracellular macromolecules, such as polypeptides in vivo, and these interacting molecules are referred to herein as “binding partners.” Binding partners can agonize or antagonize target molecule biological activity. Also, test molecules that agonize or antagonize interactions between target molecules and binding partners can be useful as therapeutic molecules as they can up-regulate or down-regulated target molecule activity in vivo and thereby treat osteoporosis.

Binding partners of target molecules can be identified by methods known in the art. For example, binding partners may be identified by lysing cells and analyzing cell lysates by electrophoretic techniques. Alternatively, a two-hybrid assay or three-hybrid assay can be utilized (see, e.g., U.S. Pat. No. 5,283,317; Zervos et al., Cell 72:223-232 (1993); Madura et al., J. Biol. Chem. 268: 12046-12054 (1993); Bartel et al., Biotechniques 14: 920-924 (1993); Iwabuchi et al., Oncogene 8: 1693-1696 (1993); and Brent WO94/10300). A two-hybrid system is based on the modular nature of most transcription factors, which consist of separable DNA-binding and activation domains. The assay often utilizes two different DNA constructs. In one construct, a CETP, PROL4, GRID2, PDE4D or GPX3/TNIP1 nucleic acid (sometimes referred to as the “bait”) is fused to a gene encoding the DNA binding domain of a known transcription factor (e.g., GAL-4). In another construct, a DNA sequence from a library of DNA sequences that encodes a potential binding partner (sometimes referred to as the “prey”) is fused to a gene that encodes an activation domain of the known transcription factor. Sometimes, a CETP, PROL4, GRID2, PDE4D or GPX3/TNIP1 nucleic acid can be fused to the activation domain. If the “bait” and the “prey” molecules interact in vivo, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to identify the potential binding partner.

In an embodiment for identifying test molecules that antagonize or agonize complex formation between target molecules and binding partners, a reaction mixture containing the target molecule and the binding partner is prepared, under conditions and for a time sufficient to allow complex formation. The reaction mixture often is provided in the presence or absence of the test molecule. The test molecule can be included initially in the reaction mixture, or can be added at a time subsequent to the addition of the target molecule and its binding partner. Control reaction mixtures are incubated without the test molecule or with a placebo. Formation of any complexes between the target molecule and the binding partner then is detected. Decreased formation of a complex in the reaction mixture containing test molecule as compared to in a control reaction mixture indicates that the molecule antagonizes target molecule/binding partner complex formation. Alternatively, increased formation of a complex in the reaction mixture containing test molecule as compared to in a control reaction mixture indicates that the molecule agonizes target molecule/binding partner complex formation. In another embodiment, complex formation of target molecule/binding partner can be compared to complex formation of mutant target molecule/binding partner (e.g., amino acid modifications in a target polypeptide). Such a comparison can be important in those cases where it is desirable to identify test molecules that modulate interactions of mutant but not non-mutated target gene products.

The assays can be conducted in a heterogeneous or homogeneous format. In heterogeneous assays, target molecule and/or the binding partner are immobilized to a solid phase, and complexes are detected on the solid phase at the end of the reaction. In homogeneous assays, the entire reaction is carried out in a liquid phase. In either approach, the order of addition of reactants can be varied to obtain different information about the molecules being tested. For example, test compounds that agonize target molecule/binding partner interactions can be identified by conducting the reaction in the presence of the test molecule in a competition format. Alternatively, test molecules that agonize preformed complexes, e.g., molecules with higher binding constants that displace one of the components from the complex, can be tested by adding the test compound to the reaction mixture after complexes have been formed.

In a heterogeneous assay embodiment, the target molecule or the binding partner is anchored onto a solid surface (e.g., a microtiter plate), while the non-anchored species is labeled, either directly or indirectly. The anchored molecule can be immobilized by non-covalent or covalent attachments. Alternatively, an immobilized antibody specific for the molecule to be anchored can be used to anchor the molecule to the solid surface. The partner of the immobilized species is exposed to the coated surface with or without the test molecule. After the reaction is complete, unreacted components are removed (e.g., by washing) such that a significant portion of any complexes formed will remain immobilized on the solid surface. Where the non-immobilized species is pre-labeled, the detection of label immobilized on the surface is indicative of complex. Where the non-immobilized species is not pre-labeled, an indirect label can be used to detect complexes anchored to the surface; e.g., by using a labeled antibody specific for the initially non-immobilized, species. Depending upon the order of addition of reaction components, test compounds that inhibit complex formation or that disrupt preformed complexes can be detected.

In another embodiment, the reaction can be conducted in a liquid phase in the presence or absence of test molecule, where the reaction products are separated from unreacted components, and the complexes are detected (e.g., using an immobilized antibody specific for one of the binding components to anchor any complexes formed in solution, and a labeled antibody specific for the other partner to detect anchored complexes). Again, depending upon the order of addition of reactants to the liquid phase, test compounds that inhibit complex or that disrupt preformed complexes can be identified.

In an alternate embodiment, a homogeneous assay can be utilized. For example, a preformed complex of the target gene product and the interactive cellular or extracellular binding partner product is prepared. One or both of the target molecule or binding partner is labeled, and the signal generated by the label(s) is quenched upon complex formation (e.g., U.S. Pat. No. 4,109,496 that utilizes this approach for immunoassays). Addition of a test molecule that competes with and displaces one of the species from the preformed complex will result in the generation of a signal above background. In this way, test substances that disrupt target molecule/binding partner complexes can be identified.

Candidate therapeutics for treating osteoporosis are identified from a group of test molecules that interact with a target molecule. Test molecules are normally ranked according to the degree with which they modulate (e.g. agonize or antagonize) a function associated with the target molecule (e.g., DNA replication and/or processing, RNA transcription and/or processing, polypeptide production and/or processing, and/or biological function/activity), and then top ranking modulators are selected. Also, pharmacogenomic information described herein can determine the rank of a modulator. The top 10% of ranked test molecules often are selected for further testing as candidate therapeutics, and sometimes the top 15%, 20%, or 25% of ranked test molecules are selected for further testing as candidate therapeutics. Candidate therapeutics typically are formulated for administration to a subject.

Therapeutic Formulations

Formulations and pharmaceutical compositions typically include in combination with a pharmaceutically acceptable carrier one or more target molecule modulators. The modulator often is a test molecule identified as having an interaction with a target molecule by a screening method described above. The modulator may be a compound, an antisense nucleic acid, a ribozyme, an antibody, or a binding partner. Also, formulations may comprise a target polypeptide or fragment thereof in combination with a pharmaceutically acceptable carrier.

As used herein, the term “pharmaceutically acceptable carrier” includes solvents, dispersion media, coatings, antibacterial and antifungal agents, isotonic and absorption delaying agents, and the like, compatible with pharmaceutical administration. Supplementary active compounds can also be incorporated into the compositions. Pharmaceutical compositions can be included in a container, pack, or dispenser together with instructions for administration.

A pharmaceutical composition typically is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art. Molecules can also be prepared in the form of suppositories (e.g., with conventional suppository bases such as cocoa butter and other glycerides) or retention enemas for rectal delivery.

In one embodiment, active molecules are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. Materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Molecules which exhibit high therapeutic indices are preferred. While molecules that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such molecules lies preferably within a range of circulating concentrations that include the ED₅₀ with little or no toxicity. The dosage may vary within this range depending upon the dosage form employed and the route of administration utilized. For any molecules used in the methods described herein, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of protein or polypeptide (i.e., an effective dosage) ranges from about 0.001 to 30 mg/kg body weight, sometimes about 0.01 to 25 mg/kg body weight, often about 0.1 to 20 mg/kg body weight, and more often about 1 to 10 mg/kg, 2 to 9 mg/kg, 3 to 8 mg/kg, 4 to 7 mg/kg, or 5 to 6 mg/kg body weight. The protein or polypeptide can be administered one time per week for between about 1 to 10 weeks, sometimes between 2 to 8 weeks, often between about 3 to 7 weeks, and more often for about 4, 5, or 6 weeks. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or, preferably, can include a series of treatments.

With regard to polypeptide formulations, featured herein is a method for treating osteoporosis in a subject, which comprises contacting one or more cells in the subject with a first polypeptide, where the subject comprises a second polypeptide having one or more polymorphic variations associated with low BMD, and where the first polypeptide comprises fewer polymorphic variations associated with low BMD than the second polypeptide. The first and second polypeptides are encoded by a nucleic acid which comprises a nucleotide sequence in SEQ ID NO's:1-10; a nucleotide sequence which encodes a polypeptide consisting of an amino acid sequence encoded by a nucleotide sequence referenced in SEQ ID NO's:1-10; a nucleotide sequence which encodes a polypeptide that is 90% or more identical to an amino acid sequence encoded by a nucleotide sequence of SEQ ID NO's:1-10 and a nucleotide sequence 90% or more identical to a nucleotide sequence in SEQ ID NO's:1-10. The subject often is a human.

For antibodies, a dosage of 0.1 mg/kg of body weight (generally 10 mg/kg to 20 mg/kg) is often utilized. If the antibody is to act in the brain, a dosage of 50 mg/kg to 100 mg/kg is often appropriate. Generally, partially human antibodies and fully human antibodies have a longer half-life within the human body than other antibodies. Accordingly, lower dosages and less frequent administration is often possible. Modifications such as lipidation can be used to stabilize antibodies and to enhance uptake and tissue penetration (e.g., into the brain). A method for lipidation of antibodies is described by Cruikshank et al., J. Acquired Immune Deficiency Syndromes and Human Retrovirology 14:193 (1997).

Antibody conjugates can be used for modifying a given biological response, the drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a polypeptide such as tumor necrosis factor, .alpha.-interferon, .beta.-interferon, nerve growth factor, platelet derived growth factor, tissue plasminogen activator; or, biological response modifiers such as, for example, lymphokines, interleukin-1 (“IL-1”), interleukin-2 (“IL-2”), interleukin-6 (“IL-6”), granulocyte macrophage colony stimulating factor (“GM-CSF”), granulocyte colony stimulating factor (“G-CSF”), or other growth factors. Alternatively, an antibody can be conjugated to a second antibody to form an antibody heteroconjugate as described by Segal in U.S. Pat. No. 4,676,980.

For compounds, exemplary doses include milligram or microgram amounts of the compound per kilogram of subject or sample weight, for example, about 1 microgram per kilogram to about 500 milligrams per kilogram, about 100 micrograms per kilogram to about 5 milligrams per kilogram, or about 1 microgram per kilogram to about 50 micrograms per kilogram. It is understood that appropriate doses of a small molecule depend upon the potency of the small molecule with respect to the expression or activity to be modulated. When one or more of these small molecules is to be administered to an animal (e.g., a human) in order to modulate expression or activity of a polypeptide or nucleic acid described herein, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

With regard to nucleic acid formulations, gene therapy vectors can be delivered to a subject by, for example, intravenous injection, local administration (see, e.g., U.S. Pat. No. 5,328,470) or by stereotactic injection (see e.g., Chen et al., (1994) Proc. Natl. Acad. Sci. USA 91:3054-3057). Pharmaceutical preparations of gene therapy vectors can include a gene therapy vector in an acceptable diluent, or can comprise a slow release matrix in which the gene delivery vehicle is imbedded. Alternatively, where the complete gene delivery vector can be produced intact from recombinant cells (e.g., retroviral vectors) the pharmaceutical preparation can include one or more cells which produce the gene delivery system. Examples of gene delivery vectors are described herein.

Therapeutic Methods

A therapeutic formulation described above can be administered to a subject in need of a therapeutic for inducing a desired biological response. Therapeutic formulations can be administered by any of the paths described herein. With regard to both prophylactic and therapeutic methods of treatment, such treatments may be specifically tailored or modified, based on knowledge obtained from pharmacogenomic analyses described herein.

As used herein, the term “treatment” is defined as the application or administration of a therapeutic formulation to a subject, or application or administration of a therapeutic agent to an isolated tissue or cell line from a subject with the purpose to cure, heal, alleviate, relieve, alter, remedy, ameliorate, improve or affect osteoporosis, symptoms of osteoporosis or a predisposition towards osteoporosis. A therapeutic formulation includes, but is not limited to, small molecules, peptides, antibodies, ribozymes and antisense oligonucleotides. Administration of a therapeutic formulation can occur prior to the manifestation of symptoms characteristic of low BMD, such that osteoporosis is prevented or delayed in its progression. The appropriate therapeutic composition can be determined based on screening assays described herein.

As discussed, successful treatment of osteoporosis can be brought about by techniques that serve to agonize target molecule expression or function, or alternatively, antagonize target molecule expression or function. These techniques include administration of modulators that include, but are not limited to, small organic or inorganic molecules; antibodies (including, for example, polyclonal, monoclonal, humanized, anti-idiotypic, chimeric or single chain antibodies, and Fab, F(ab′)₂ and Fab expression library fragments, scFV molecules, and epitope-binding fragments thereof); and peptides, phosphopeptides, or polypeptides.

Further, antisense and ribozyme molecules that inhibit expression of the target gene can also be used to reduce the level of target gene expression, thus effectively reducing the level of target gene activity. Still further, triple helix molecules can be utilized in reducing the level of target gene activity. Antisense, ribozyme and triple helix molecules are discussed above. It is possible that the use of antisense, ribozyme, and/or triple helix molecules to reduce or inhibit mutant gene expression can also reduce or inhibit the transcription (triple helix) and/or translation (antisense, ribozyme) of mRNA produced by normal target gene alleles, such that the concentration of normal target gene product present can be lower than is necessary for a normal phenotype. In such cases, nucleic acid molecules that encode and express target gene polypeptides exhibiting normal target gene activity can be introduced into cells via gene therapy method. Alternatively, in instances in that the target gene encodes an extracellular polypeptide, it can be preferable to co-administer normal target gene polypeptide into the cell or tissue in order to maintain the requisite level of cellular or tissue target gene activity.

Another method by which nucleic acid molecules may be utilized in treating or preventing osteoporosis is use of aptamer molecules specific for target molecules. Aptamers are nucleic acid molecules having a tertiary structure which permits them to specifically bind to ligands (see, e.g., Osborne, et al., Curr. Opin. Chem. Biol. 1(1): 5-9 (1997); and Patel, D. J., Curr. Opin. Chem. Biol. June; 1(1): 32-46 (1997)).

Yet another method of utilizing nucleic acid molecules for osteoporosis treatment is gene therapy, which can also be referred to as allele therapy. Provided herein is a gene therapy method for treating osteoporosis in a subject, which comprises contacting one or more cells in the subject or from the subject with a nucleic acid having a first nucleotide sequence. Genomic DNA in the subject comprises a second nucleotide sequence having one or more polymorphic variations associated with low BMD (e.g., the second nucleic acid has a nucleotide sequence in SEQ ID NO's:1-10). The first and second nucleotide sequences typically are substantially identical to one another, and the first nucleotide sequence comprises fewer polymorphic variations associated with low BMD than the second nucleotide sequence. The first nucleotide sequence may comprise a gene sequence that encodes a full-length polypeptide or a fragment thereof. The subject is often a human. Allele therapy methods often are utilized in conjunction with a method of first determining whether a subject has genomic DNA that includes polymorphic variants associated with low BMD.

In another allele therapy embodiment, provided herein is a method which comprises contacting one or more cells in the subject or from the subject with a polypeptide encoded by a nucleic acid having a first nucleotide sequence. Genomic DNA in the subject comprises a second nucleotide sequence having one or more polymorphic variations associated with low BMD (e.g., the second nucleic acid has a nucleotide sequence in SEQ ID NO's:1-10). The first and second nucleotide sequences typically are substantially identical to one another, and the first nucleotide sequence comprises fewer polymorphic variations associated with low BMD than the second nucleotide sequence. The first nucleotide sequence may comprise a gene sequence that encodes a full-length polypeptide or a fragment thereof. The subject is often a human.

For antibody-based therapies, antibodies can be generated that are both specific for target molecules and that reduce target molecule activity. Such antibodies may be administered in instances where antagonizing a target molecule function is appropriate for the treatment of osteoporosis.

In circumstances where stimulating antibody production in an animal or a human subject by injection with a target molecule is harmful to the subject, it is possible to generate an immune response against the target molecule by use of anti-idiotypic antibodies (see, e.g., Herlyn, Ann. Med.; 31(1): 66-78 (1999); and Bhattacharya-Chatterjee & Foon, Cancer Treat. Res.; 94: 51-68 (1998)). Introducing an anti-idiotypic antibody to a mammal or human subject often stimulates production of anti-anti-idiotypic antibodies, which typically are specific to the target molecule. Vaccines directed to osteoporosis also may be generated in this fashion.

In instances where the target molecule is intracellular and whole antibodies are used, internalizing antibodies may be preferred. Lipofectin or liposomes can be used to deliver the antibody or a fragment of the Fab region that binds to the target antigen into cells. Where fragments of the antibody are used, the smallest inhibitory fragment that binds to the target antigen is preferred. For example, peptides having an amino acid sequence corresponding to the Fv region of the antibody can be used. Alternatively, single chain neutralizing antibodies that bind to intracellular target antigens can also be administered. Such single chain antibodies can be administered, for example, by expressing nucleotide sequences encoding single-chain antibodies within the target cell population (see, e.g., Marasco et al., Proc. Natl. Acad. Sci. USA 90: 7889-7893 (1993)).

Modulators can be administered to a patient at therapeutically effective doses to treat osteoporosis. A therapeutically effective dose refers to an amount of the modulator sufficient to result in amelioration of symptoms of osteoporosis. Toxicity and therapeutic efficacy of modulators can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Modulators that exhibit large therapeutic indices are preferred. While modulators that exhibit toxic side effects can be used, care should be taken to design a delivery system that targets such molecules to the site of affected tissue in order to minimize potential damage to uninfected cells, thereby reducing side effects.

Data obtained from cell culture assays and animal studies can be used in formulating a range of dosages for use in humans. The dosage of such compounds lies preferably within a range of circulating concentrations that include the ED₅₀ with little or no toxicity. The dosage can vary within this range depending upon the dosage form employed and the route of administration utilized. For any compound used in the methods described herein, the therapeutically effective dose can be estimated initially from cell culture assays. A dose can be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma can be measured, for example, by high performance liquid chromatography.

Another example of effective dose determination for an individual is the ability to directly assay levels of “free” and “bound” compound in the serum of the test subject. Such assays may utilize antibody mimics and/or “biosensors” that have been created through molecular imprinting techniques. Molecules that modulate target molecule activity are used as a template, or “imprinting molecule”, to spatially organize polymerizable monomers prior to their polymerization with catalytic reagents. The subsequent removal of the imprinted molecule leaves a polymer matrix which contains a repeated “negative image” of the compound and is able to selectively rebind the molecule under biological assay conditions. A detailed review of this technique can be seen in Ansell et al., Current Opinion in Biotechnology 7: 89-94 (1996) and in Shea, Trends in Polymer Science 2: 166-173 (1994). Such “imprinted” affinity matrixes are amenable to ligand-binding assays, whereby the immobilized monoclonal antibody component is replaced by an appropriately imprinted matrix. An example of the use of such matrixes in this way can be seen in Vlatakis, et al., Nature 361: 645-647 (1993). Through the use of isotope-labeling, the “free” concentration of compound which modulates target molecule expression or activity readily can be monitored and used in calculations of IC₅₀. Such “imprinted” affinity matrixes can also be designed to include fluorescent groups whose photon-emitting properties measurably change upon local and selective binding of target compound. These changes readily can be assayed in real time using appropriate fiberoptic devices, in turn allowing the dose in a test subject to be quickly optimized based on its individual IC₅₀. An example of such a “biosensor” is discussed in Kriz et al., Analytical Chemistry 67: 2142-2144 (1995).

The examples set forth below are intended to illustrate but not limit the invention.

### EXAMPLES

In the following studies a group of subjects were selected according to specific parameters relating to low BMD. Nucleic acid samples obtained from individuals in the study group were subjected to genetic analysis, which identified associations between low BMD and certain polymorphic variants in CETP, PROL4, GRID2, PDE4D and GPX3 (herein referred to as “target genes”, “target nucleotides”, “target polypeptides” or simply “targets”). Methods are described for producing CETP, PROL4, GRID2, PDE4D and GPX3 polypeptides and polypeptide variants in vitro or in vivo. CETP, PROL4, GRID2, PDE4D and GPX3 nucleic acids or polypeptides and variants thereof are utilized for screening test molecules for those that interact with CETP, PROL4, GRID2, PDE4D and GPX3 molecules. Test molecules identified as being interactors with target polypeptides can be screened further as osteoporosis therapeutics.

### Example 1

### Samples and Pooling Strategies

Sample Selection

Blood samples were collected from individuals with low BMD, which were referred to as case samples. Also, blood samples were collected from individuals with high BMD (i.e., not diagnosed with osteoporosis or any form of osteoporosis-related disease); these samples served as gender and age-matched controls. All of the samples were of Caucasian (English) descent A database was created that listed all phenotypic trait information gathered from individuals for each case and control sample. Genomic DNA was extracted from each of the blood samples for genetic analyses.

DNA Extraction from Blood Samples

Six to ten milliliters of whole blood was transferred to a 50 ml tube containing 27 ml of red cell lysis solution (RCL). The tube was inverted until the contents were mixed. Each tube was incubated for 10 minutes at room temperature and inverted once during the incubation. The tubes were then centrifuged for 20 minutes at 3000×g and the supernatant was carefully poured off. 100-200 μl of residual liquid was left in the tube and was pipetted repeatedly to resuspend the pellet in the residual supernatant White cell lysis solution (WCL) was added to the tube and pipetted repeatedly until completely mixed. While no incubation was normally required, the solution was incubated at 37° C. or room temperature if cell clumps were visible after mixing until the solution was homogeneous. 2 ml of protein precipitation was added to the cell lysate. The mixtures were vortexed vigorously at high speed for 20 sec to mix the protein precipitation solution uniformly with the cell lysate, and then centrifuged for 10 minutes at 3000×g. The supernatant containing the DNA was then poured into a clean 15 ml tube, which contained 7 ml of 100% isopropanol. The samples were mixed by inverting the tubes gently until white threads of DNA were visible. Samples were centrifuged for 3 minutes at 2000×g and the DNA was visible as a small white pellet. The supernatant was decanted and 5 ml of 70% ethanol was added to each tube. Each tube was inverted several times to wash the DNA pellet, and then centrifuged for 1 minute at 2000×g. The ethanol was decanted and each tube was drained on clean absorbent paper. The DNA was dried in the tube by inversion for 10 minutes, and then 1000 μl of 1×TE was added. The size of each sample was estimated, and less TE buffer was added during the following DNA hydration step if the sample was smaller. The DNA was allowed to rehydrate overnight at room temperature, and DNA samples were stored at 2-8° C.

DNA was quantified by placing samples on a hematology mixer for at least 1 hour. DNA was serially diluted (typically 1:80, 1:160, 1:320, and 1:640 dilutions) so that it would be within the measurable range of standards. 125 μl of diluted DNA was transferred to a clear U-bottom microtitre plate, and 125 μl of 1×TE buffer was transferred into each well using a multichannel pipette. The DNA and 1×TE were mixed by repeated pipetting at least 15 times, and then the plates were sealed. 50 μl of diluted DNA was added to wells A5-H12 of a black flat bottom microtitre plate. Standards were inverted six times to mix them, and then 50 μl of 1×TE buffer was pipetted into well A1, 1000 ng/ml of standard was pipetted into well A2, 500 ng/ml of standard was pipetted into well A3, and 250 ng/ml of standard was pipetted into well A4. PicoGreen (Molecular Probes, Eugene, Oreg.) was thawed and freshly diluted 1:200 according to the number of plates that were being measured. PicoGreen was vortexed and then 50 μl was pipetted into all wells of the black plate with the diluted DNA. DNA and PicoGreen were mixed by pipetting repeatedly at least 10 times with the multichannel pipette. The plate was placed into a Fluoroskan Ascent Machine (microplate fluorometer produced by Labsystems) and the samples were allowed to incubate for 3 minutes before the machine was run using filter pairs 485 nm excitation and 538 nm emission wavelengths. Samples having measured DNA concentrations of greater than 450 ng/l were re-measured for conformation. Samples having measured DNA concentrations of 20 ng/μl or less were re-measured for confirmation.

Pooling Strategies

Samples were placed into one of two groups based on BMD levels. The two groups were made up of individuals with low BMD levels and individuals with high BMD levels. A select set of samples from each group were utilized to generate pools, and one pool was created for each group. Each individual sample in a pool was represented by an equal amount of genomic DNA. For example, where 25 ng of genomic DNA was utilized in each PCR reaction and there were 200 individuals in each pool, each individual would provide 125 pg of genomic DNA. Inclusion or exclusion of samples for a pool was based upon the following criteria: the sample was derived from an individual of Caucasian paternal and maternal descent; the database included relevant phenotype information for the individual; case samples were derived from individuals with low BMD; control samples were derived from individuals with normal or high BMD and no history of osteoporosis or osteoporosis-related diseases; and sufficient genomic DNA was extracted from each blood sample for all allelotyping and genotyping reactions performed during the study. Phenotype information included pre- or post-menopausal, familial predisposition, country or origin of mother and father, diagnosis with osteoporosis (date of primary diagnosis, age of individual as of primary diagnosis, osteoporosis-related fracture), biochemical measurements of markers of bone resorption (bone-specific alkaline Phosphatase, Urinary C-telopeptide of type I collagen, serum osteocalcin), current medication status (thyroid medication, hormone replacement therapy, steroid usage, bisphosphonates and cytotoxic agents for rheumatic diseases). Samples that met the inclusion criteria and did not meet the exclusion criteria were added to appropriate pools based on gender and disease status.

The selection process yielded the pools set forth in Table 2, which were used in the studies that follow. The average (mean) T-score in the High group is 1.56, and in the Low group −2.1 which means that on average individuals in the high BMD pool are 1.56 standard deviations above the average BMD in young females, whereas individuals in the low BMD pool are on average 2.1 standard deviations below the normal young value.

TABLE 2

| | Female High BMD | Female Low BMD |
|---|---|---|
| Pool size | 321 | 319 |
| (Number) | | |
| Mean T-score | 1.56 | −2.10 |
| (BMD leveles adjusted for | | |
| age and BMI) | | |
| Mean Age | 52.9 | 52.4 |
| (ex: years) | | |

### Example 2

### Association of Polymorphic Variants with Low BMD

A whole-genome screen was performed to identify particular SNPs associated with low BMD. As described in Example 1, two groups of samples were utilized, which included samples from female individuals having low BMD (osteoporosis cases), and samples from female individuals having high BMD levels (controls). The initial screen of each pool was performed in an allelotyping study, in which certain samples in each group were pooled. By pooling DNA from each group, an allele frequency for each SNP in each group was calculated. These allele frequencies were then compared to one another. Particular SNPs were considered as being associated with low BMD when allele frequency differences calculated between case and control pools were statistically significant. SNP disease association results obtained from the allelotyping study were then validated by genotyping each associated SNP across all samples from each pool. The results of the genotyping were then analyzed, allele frequencies for each group were calculated from the individual genotyping results, and a p-value was calculated to determine whether the case and control groups had statistically significantly differences in allele frequencies for a particular SNP. When the genotyping results agreed with the original allelotyping results, the SNP disease association was considered validated at the genetic level.

SNP Panel Used for Genetic Analyses

A whole-genome SNP screen began with an initial screen of approximately 25,000 SNPs over each set of disease and control samples using a pooling approach. The pools studied in the screen are described in Example 1. The SNPs analyzed in this study were part of a set of 25,488 SNPs confirmed as being statistically polymorphic as each is characterized as having a minor allele frequency of greater than 10%. The SNPs in the set reside in genes or in close proximity to genes, and many reside in gene exons. Specifically, SNPs in the set are located in exons, introns, and within 5,000 base-pairs upstream of a transcription start site of a gene. In addition, SNPs were selected according to the following criteria: they are located in ESTs; they are located in Locuslink or Ensemb1 genes; and they are located in Genomatix promoter predictions. SNPs in the set were also selected on the basis of even spacing across the genome, as depicted in Table 3. An additional 3088 SNPs were included with these 25,488 SNPs and these additional SNPs had been chosen on the basis of gene location, with preference to non-synonymous coding SNPs located in disease candidate genes.

TABLE 3

| General Statistics | | Spacing Statistics | |
|---|---|---|---|
| Total # of SNPs | 25,488 | Median | 37,058 bp |
| # of Exonic SNPs | >4,335 (17%) | Minimum* | 1,000 bp |
| # SNPs with | 20,776 (81%) | Maximum* | 3,000,000 bp |
| refSNP ID | | | |
| Gene Coverage | >10,000 | Mean | 122,412 bp |
| Chromosome | All | Std Deviation | 373,325 pb |
| Coverage | | | |

*Excludes outliers

Allelotyping and Genotyping Results

The genetic studies summarized above and described in more detail below identified allelic variants associated with low BMD. The allelic variants identified from the SNP panel described in Table 3 are summarized below in Table 4.

TABLE 4

| | | | | | | | | | | Low |
|---|---|---|---|---|---|---|---|---|---|---|
| | | | Position | | | | | | | BMD |
| SNP | | Chromosome | in SEQ | Contig | Contig | Sequence | | Sequence | Allelic | Assoc. |
| Reference | Chromosome | Position | ID Nos | Identification | Position | Identification | Locus | Position | Variability | Allele |
| rs1801706 | 16q21 | 57068823 | 50109 | NT_010498 | 10631861 | NM_000078 | CETP | UTR | AG | G |
| rs1047699 | 12p13 | 11143886 | 49075 | NT_009714 | 3758682 | NM_007244 | PROL4 | Exon: | CT | C |
| | | | | | | | | R120Q | | |
| rs1948017 | 4q22 | 94636365 | 49110 | NT_016354 | 18829520 | NM_001510 | GRID2 | Intron | CT | C |
| rs1498608 | 5q12 | 59895580 | 49652 | NT_006713 | 8937426 | NM_006203 | PDE4D | Intron | AT | T |
| rs869975 | 5q23 | 150475233 | 50082 | NT_029289 | 11569308 | NM_002084 | GPX3 | Intron | AG | G |

Table 4 includes information pertaining to the incident polymorphic variant associated with low BMD identified herein. Public information pertaining to the polymorphism and the genomic sequence that includes the polymorphism are indicated. The genomic sequences identified in Table 4 may be accessed at the http address www.ncbi.nih.gov/entrez/query.fcgi, for example, by using the publicly available SNP reference number (e.g., rs1801706). The chromosome position refers to the position of the SNP within NCBI's Genome Build 34, which may be accessed at the following http address: www.ncbi.nlm.nih.gov/mapview/map_search.cgi?chr=hum_chr.inf&query=. The “Contig Position” provided in Table 4 corresponds to a nucleotide position set forth in the contig sequence, and designates the polymorphic site corresponding to the SNP reference number. The sequence containing the polymorphisms also may be referenced by the “Sequence Identification” set forth in Table 4. The “Sequence Identification” corresponds to cDNA sequence that encodes associated target polypeptides (e.g., CETP) of the invention. The position of the SNP within the cDNA sequence is provided in the “Sequence Position” column of Table 4. Also, the allelic variation at the polymorphic site and the allelic variant identified as associated with low BMD is specified in Table 4. All nucleotide sequences referenced and accessed by the parameters set forth in Table 4 are incorporated herein by reference.

Assay for Verifying, Allelotyping, and Genotyping SNPs

A MassARRAY® system (Sequenom, Inc.) was utilized to perform SNP genotyping in a high-throughput fashion. This genotyping platform was complemented by a homogeneous, single-tube assay method (hME™ or homogeneous MassEXTEND™ (Sequenom, Inc.)) in which two genotyping primers anneal to and amplify a genomic target surrounding a polymorphic site of interest. A third primer (the MassEXTEND™ primer), which is complementary to the amplified target up to but not including the polymorphism, was then enzymatically extended one or a few bases through the polymorphic site and then terminated.

For each polymorphism, SpectroDESIGNER™ software (Sequenom, Inc.) was used to generate a set of PCR primers and a MassEXTEND™ primer was used to genotype the polymorphism. Table 5 shows PCR primers and Table 6 shows extension primers used for analyzing polymorphisms. The initial PCR amplification reaction was performed in a 5 A1 total volume containing 1×PCR buffer with 1.5 mM MgCl₂ (Qiagen), 200 μM each of dATP, dGTP, dCTP, dTTP (Gibco-BRL), 2.5 ng of genomic DNA, 0.1 units of HotStar DNA polymerase (Qiagen), and 200 nM each of forward and reverse PCR primers specific for the polymorphic region of interest.

TABLE 5
PCR Primers

| Reference | Forward | Reverse |
|---|---|---|
| SNP ID | PCR primer | PCR primer |
| rs1801706 | ACGTTGGATGTTGTAGCAG | ACGTTGGATGTCCATCTCCG |
| | AAGGCAAGCAC | TACTCCTAAC |
| rs1047699 | GATTACCAGAGTGGTTGCT | CCTGCAGGAAGCATCATCAT |
| | C | |
| rs1948017 | GTTTAACAGCAACCATTGA | CCCCAAAGGTATGTTAAGAG |
| | GG | |
| rs1498608 | GAATCCCTGTTCATTCCTT | ATACCTAGGTATAACCTCGG |
| | G | |
| rs869975 | AACTCACTGGTGATCCTGC | TGTCTCATCCACACCACTCC |
| | G | |

Samples were incubated at 95° C. for 15 minutes, followed by 45 cycles of 95° C. for 20 seconds, 56° C. for 30 seconds, and 72° C. for 1 minute, finishing with a 3 minute final extension at 720° C. Following amplification, shrimp alkaline phosphatase (SAP) (0.3 units in a 2 μl volume) (Amersham Pharmacia) was added to each reaction (total reaction volume was 7 μl) to remove any residual dNTPs that were not consumed in the PCR step. Samples were incubated for 20 minutes at 37° C., followed by 5 minutes at 85° C. to denature the SAP.

Once the SAP reaction was complete, a primer extension reaction was initiated by adding a polymorphism-specific MassEXTEND™ primer cocktail to each sample. Each MassEXTEND™ cocktail included a specific combination of dideoxynucleotides (ddNTPs) and deoxynucleotides (dNTPs) used to distinguish polymorphic alleles from one another. In Table 6, ddNTPs are shown and the fourth nucleotide not shown is the dNTP.

TABLE 6
Extend Primers

| Reference | Extend | Term |
|---|---|---|
| SNP ID | Probe | Mix |
| rs1801708 | CCTGGTGTCTCCTCCAGC | ACT |
| rs1047699 | TGTCTTGCTGGTCTGTCCCTC | ACG |
| rs1948017 | CAGCAACCATTGAGGGTGAAT | ACG |
| rs1498608 | CCCTAAAAACTGTTCCAGGTA | CGT |
| rs869975 | GGGCCTCAGTAGTTCCAGC | ACT |

The MassEXTEND™ reaction was performed in a total volume of 9 μl, with the addition of 1× ThermoSequenase buffer, 0.576 units of ThermoSequenase (Amersham Pharmacia), 600 nM MassEXTEND™ primer, 2 mM of ddATP and/or ddCTP and/or ddGTP and/or ddTTP, and 2 mM of dATP or dCTP or dGTP or dTTP. The deoxy nucleotide (dNTP) used in the assay normally was complementary to the nucleotide at the polymorphic site in the amplicon. Samples were incubated at 94° C. for 2 minutes, followed by 55 cycles of 5 seconds at 94° C., 5 seconds at 52° C., and 5 seconds at 72° C.

Following incubation, samples were desalted by adding 16 μl of water (total reaction volume was 25 μl), 3 mg of SpectroCLEAN™ sample cleaning beads (Sequenom, Inc.) and allowed to incubate for 3 minutes with rotation. Samples were then robotically dispensed using a piezoelectric dispensing device (SpectroJET™ (Sequenom, Inc.)) onto either 96-spot or 384-spot silicon chips containing a matrix that crystallized each sample (SpectroCHIP® (Sequenom, Inc.)). Subsequently, MALDI-TOF mass spectrometry (Biflex and Autoflex MALDI-TOF mass spectrometers (Bruker Daltonics) can be used) and SpectroTYPER RT™ software (Sequenom, Inc.) were used to analyze and interpret the SNP genotype for each sample.

Genetic Analysis

Variations identified in the target genes are provided in their respective genomic sequences (see SEQ ID NOs:1-4) Minor allelic frequencies for these polymorphisms was verified as being 10% or greater by determining the allelic frequencies using the extension assay described above in a group of samples isolated from 92 individuals originating from the state of Utah in the United States, Venezuela and France (Coriell cell repositories).

Genotyping results for the allelic variant set forth in Table 4 are shown for female pools in Table 7. In Table 7, “F case” and “F control” refer to female case (low BMD) and female control (high BMD) groups, and “AF” refers to allele frequency.

TABLE 7
Female Genotype Results

| | | | | | Low BMD |
|---|---|---|---|---|---|
| SNP | F AF case | F AF control | | Odds | Associated |
| Reference | (low) | (high) | p-value | Ratio | Allele |
| ra1801706 | A = 0.15 | A = 0.19 | 0.00765 | 0.661 | G |
| | G = 0.85 | G = 0.81 | | | |
| rs1047699 | C = 0.85 | C = 0.78 | 0.000594 | 1.65 | C |
| | T = 0.15 | T = 0.22 | | | |
| rs1948017 | C = 0.14 | C = 0.10 | 0.0188 | 1.51 | C |
| | T = 0.86 | T = 0.90 | | | |
| rs1498608 | A = 0.09 | A = 0.12 | 0.0347 | 0.672 | T |
| | T = 0.91 | T = 0.88 | | | |
| rs869975 | A = 0.05 | A = 0.10 | 0.000243 | 0.441 | G |
| | G = 0.95 | G = 0.90 | | | |

The single marker alleles set forth in Table 7 were considered validated, since the genotyping data were significantly associated with low BMD, and because the genotyping results agreed with the original allelotyping results. Particularly significant associations with low BMD are indicated by a calculated p-value of less than 0.05 for genotype results, which are set forth in bold text.

Odds ratio results are shown in Table 7 (and other Tables below). An odds ratio is an unbiased estimate of relative risk which can be obtained from most case-control studies. Relative risk (RR) is an estimate of the likelihood of disease in the exposed group (susceptibility allele or genotype carriers) compared to the unexposed group (not carriers). It can be calculated by the following equation:
RR=IA/Ia

IA is the incidence of disease in the A carriers and Ia is the incidence of disease in the non-carriers.

RR>1 indicates the A allele increases disease susceptibility.

RR<1 indicates the a allele increases disease susceptibility.

For example, RR=1.5 indicates that carriers of the A allele have 1.5 times the risk of disease than non-carriers, i.e., 50% more likely to get the disease.

Case-control studies do not allow the direct estimation of IA and Ia, therefore relative risk cannot be directly estimated. However, the odds ratio (OR) can be calculated using the following equation:
OR=(nDAnda)/(ndAnDa)=pDA(1−pdA)/pdA(1−pDA), or

OR=((case f)/(1−case f)/((control f)/(1−control f)), where f=susceptibility allele frequency.

An odds ratio can be interpreted in the same way a relative risk is interpreted and can be directly estimated using the data from case-control studies, i.e., case and control allele frequencies. The higher the odds ratio value, the larger the effect that particular allele has on the development of low BMD. Possessing an allele associated with a relatively high odds ratio translates to having a higher risk of developing or having low BMD.

### Example 3

### CETP Region Proximal SNPs

It has been discovered that a polymorphic variation (rs1801706) in a gene encoding CETP is associated with the occurrence of low BMD (see Examples 1 and 2). Ninety-one additional allelic variants proximal to rs1801706 were identified and subsequently allelotyped in low BMD case and high BMD control sample sets as described in Examples 1 and 2. The polymorphic variants are set forth in Table 8. The chromosome positions provided in column four of Table 8 are based on Genome “Build 34” of NCBI's GenBank. The “genome letter” corresponds to the particular allele that appears in NCBI's build 34 genomic sequence of the region (chromosome 16: positions 56743155-56840953), and the “deduced iupac” corresponds to the single letter IUPAC code for the CTEP polymorphic variants as they appear in SEQ ID NO:1. Also, the “genome letter” may differ from the alleles (A1/A2) provided in Table 8 (and in subsequent Tables that provide the same information) if the genome letter is on one stand and the alleles are on the complementary strand, thus having different strand orientations (i.e., reverse vs forward).

TABLE 8

| | Position in | | Chromosome | Alleles | Genome | Deduced |
|---|---|---|---|---|---|---|
| dbSNP | SEQ ID NO: 1 | Chromosome | Position | (A1/A2) | letter | iupac |
| 7500979 | 205 | 16 | 56743155 | g/a | g | R |
| 2217332 | 1595 | 16 | 56744545 | g/a | g | R |
| 8044804 | 2650 | 16 | 56745600 | c/t | a | R |
| 2270835 | 5496 | 16 | 56748446 | c/t | t | Y |
| 2133783 | 5782 | 16 | 56748732 | g/a | a | R |
| 247609 | 5908 | 16 | 56748858 | c/t | g | R |
| 952440 | 7552 | 16 | 56750502 | g/a | a | R |
| 881598 | 9191 | 16 | 56752141 | t/c | g | R |
| 2291955 | 10127 | 16 | 56753077 | g/a | t | Y |
| 2518054 | 10345 | 16 | 56753295 | g/a | g | R |
| 866038 | 10399 | 16 | 56753349 | t/c | c | Y |
| 1436425 | 12028 | 16 | 56754978 | g/a | a | R |
| 173537 | 13355 | 16 | 56756305 | a/g | t | Y |
| 247611 | 13687 | 16 | 56756637 | a/g | g | R |
| 166017 | 14328 | 16 | 56757278 | t/c | c | Y |
| 173538 | 14746 | 16 | 56757696 | c/t | t | Y |
| 193694 | 14996 | 16 | 56757946 | t/c | c | Y |
| 7205692 | 19361 | 16 | 56762311 | g/a | a | R |
| 8048746 | 21775 | 16 | 56764725 | g/a | a | R |
| 247618 | 23250 | 16 | 56766200 | g/a | g | R |
| 183130 | 23810 | 16 | 56766760 | c/t | c | Y |
| 6499863 | 24464 | 16 | 56767414 | g/a | g | R |
| 4783961 | 27341 | 16 | 56770291 | g/a | g | R |
| 3816117 | 28605 | 16 | 56771555 | c/t | t | Y |
| 711752 | 28658 | 16 | 56771608 | g/a | g | R |
| 708272 | 28735 | 16 | 56771685 | c/t | g | R |
| 1864163 | 29680 | 16 | 56772630 | g/a | g | R |
| 4369653 | 29998 | 16 | 56772948 | g/a | c | Y |
| 1864165 | 32521 | 16 | 56775471 | c/t | c | Y |
| 891141 | 36170 | 16 | 56779120 | t/g | g | K |
| 891143 | 36427 | 16 | 56779377 | c/t | t | Y |
| 7205804 | 37336 | 16 | 56780286 | g/a | g | R |
| 5885 | 37718 | 16 | 56780668 | c/t | c | Y |
| 1532625 | 37748 | 16 | 56780698 | a/g | c | Y |
| 1532624 | 37926 | 16 | 56780876 | t/g | c | M |
| 289712 | 38752 | 16 | 56781702 | g/a | c | Y |
| 7499892 | 39037 | 16 | 56781987 | c/t | c | Y |
| 5883 | 39800 | 16 | 56782750 | c/t | c | Y |
| 289714 | 39898 | 16 | 56782848 | c/t | g | R |
| 158480 | 40674 | 16 | 56783624 | c/t | g | R |
| 289717 | 41835 | 16 | 56784785 | c/t | g | R |
| 4344729 | 42325 | 16 | 56785275 | c/t | g | R |
| 289718 | 42379 | 16 | 56785329 | a/g | c | Y |
| 289719 | 42388 | 16 | 56785338 | g/a | t | Y |
| 2033254 | 42432 | 16 | 56785382 | c/t | t | Y |
| 4784744 | 43632 | 16 | 56786582 | g/a | g | R |
| 291044 | 43899 | 16 | 56786849 | c/t | g | R |
| 8053613 | 44273 | 16 | 56787223 | c/t | c | Y |
| 5881 | 44459 | 16 | 56787409 | a/g | g | R |
| 5880 | 47538 | 16 | 56790488 | c/g | g | S |
| 7198026 | 47692 | 16 | 56790642 | c/t | t | Y |
| 5882 | 48539 | 16 | 56791489 | g/a | g | R |
| 8045701 | 48749 | 16 | 56791699 | c/t | t | Y |
| 289741 | 49921 | 16 | 56792871 | a/g | g | R |
| 1801706 | 50109 | 16 | 56793059 | a/g | g | R |
| 289742 | 50209 | 16 | 56793159 | c/g | c | S |
| 289743 | 50243 | 16 | 56793193 | c/t | g | R |
| 289746 | 52652 | 16 | 56795602 | g/a | c | Y |
| 172337 | 55195 | 16 | 56798145 | c/t | t | Y |
| 289747 | 56385 | 16 | 56799335 | a/g | c | Y |
| 1566439 | 57109 | 16 | 56800059 | a/g | t | Y |
| 7205459 | 57618 | 16 | 56800568 | c/t | t | Y |
| 289749 | 58741 | 16 | 56801691 | t/c | g | R |
| 289751 | 59222 | 16 | 56802172 | t/c | g | R |
| 8059220 | 60771 | 16 | 56803721 | c/t | c | Y |
| 8058353 | 60962 | 16 | 56803912 | g/a | g | R |
| 289735 | 62009 | 16 | 56804959 | a/g | c | Y |
| 289737 | 64589 | 16 | 56807539 | t/g | a | M |
| 291042 | 66054 | 16 | 56809004 | g/a | a | R |
| 1875236 | 66143 | 16 | 56809093 | c/t | g | R |
| 821466 | 67822 | 16 | 56810772 | a/g | t | Y |
| 821465 | 68805 | 16 | 56811755 | c/g | c | S |
| 4275846 | 70075 | 16 | 56813025 | c/t | g | R |
| 289707 | 70350 | 16 | 56813300 | a/g | t | Y |
| 821463 | 71214 | 16 | 56814164 | g/a | t | Y |
| 289706 | 79549 | 16 | 56822499 | c/t | c | Y |
| 1167741 | 82760 | 16 | 56825710 | c/t | c | Y |
| 2052880 | 86463 | 16 | 56829413 | a/c | g | K |
| 1167742 | 86533 | 16 | 56829483 | c/g | c | S |
| 1183256 | 87019 | 16 | 56829969 | a/g | a | R |
| 1651665 | 88910 | 16 | 56831860 | a/c | a | M |
| 1651666 | 88955 | 16 | 56831905 | c/t | c | Y |
| 4784751 | 89021 | 16 | 56831971 | c/t | c | Y |
| 1651667 | 89056 | 16 | 56832006 | a/g | a | R |
| 8052091 | 89863 | 16 | 56832813 | g/a | g | R |
| 1684574 | 89879 | 16 | 56832829 | t/c | t | Y |
| 1684575 | 90066 | 16 | 56833016 | t/g | g | K |
| 1672865 | 90101 | 16 | 56833051 | a/g | a | R |
| 821470 | 91029 | 16 | 56833979 | g/a | a | R |
| 1549669 | 91434 | 16 | 56834384 | t/g | a | M |
| 291040 | 93636 | 16 | 56836586 | t/c | t | Y |
| 289754 | 98003 | 16 | 56840953 | c/t | c | Y |

Assay for Verifying and Allelotyping SNPs

The methods used to verify and allelotype the proximal SNPs of Table 8 are the same methods described in Examples 1 and 2 herein. The primers and probes used in these assays are provided in Table 9 and Table 10, respectively.

TABLE 9

| | | |
|---|---|---|
| dbSNP | | |
| rs# | Forward PCR primer | Reverse PCR primer |

| | | |
|---|---|---|
| 7500979 | TTGTATATGTAGGGTCCTGC | AAAATAATTCAAAACCACTG |
| 2217332 | ATCCAACAACAGCTTCCCAG | GGTGAAATGCTGACCTGTGT |
| 8044804 | ATAAATACAACCCAGCCCAC | CATTTCATTTCCCTGCACTG |
| 2270835 | CATTTACCATGTAACTGCCG | GGGTTGAGTATGAACAAATG |
| 2133783 | ACACTCATCCCTCCATTCTG | TCCCGGGAATTGAAGGGAAT |
| 247609 | GTCACTAAACTATGTATCAG | GGGTAGAGTGTAAATGACAG |
| 952440 | AGTCAGTGCCTGACTTTACC | CATACTGAGGCATGGAACAG |
| 881598 | ACTGCACTCCAGCCTGGGTGA | TGCTCCTGGCAGCAAATATC |
| 2291955 | GCTAGACATGTTTTAGCAGG | CCCCTTAGTTTAAAGTAAAGC |
| 2518054 | CTGATCATTCTTACCGGCAC | CTAGACAGTCAACAACTGAG |
| 866038 | CAAGGAACAAAGCAAGACCC | GTGCCGGTAAGAATGATCAG |
| 1436425 | GTATTCAGAGTGGTGTGTGG | GGGACATGGCAGAAATTCAG |
| 173537 | TCCCTGTCCTTGAACTCATC | CCTCTTGGGTCTTGTAGTCG |
| 247611 | AGGGTCCTCCATGATTGGAG | TTTCTGGACCTGACTGGGTG |
| 166017 | TAGATGGGCTGTTTCTACTG | AGGGAAGATTCCAGTAAAGC |
| 173538 | TCCCAAAGTGCTGGGATTAC | GGGTTTCTCAAAGGGCTAAG |
| 193694 | TCCTTCCGTGATCTCACAAC | CACACCAAAAGAACTGCATG |
| 7205692 | TCTTAGCCTGGGACTTTCTG | AAAAGCAGCTGTGACCTAAG |
| 8048746 | CAAGTTCTTCCTCCATCCAC | TTCCTCTCTCTGGGCTTATC |
| 247618 | TTTTCCCCCTTTTTGGGGC | TGGGTTGTCACAGCAAGGTGG |
| 183130 | ATAAACGGGAAAGAAGAGAC | AGGGTGGAGAATCTACAGAA |
| 6499863 | GTCTTGACAGGTTTGAGGAG | CATCAGACAGATCCCAACAG |
| 4783961 | GAAACATGAGTCGGGATGGC | AGCTTTGGTATTGGAGCAGG |
| 3816117 | ACTAGCCCAGAGAGAGGAGT | AAGAACTTGACCTTGAAGGC |
| 711752 | CACAAATCCCTATACCTGGC | TCGCCTTCAAGGTCAAGTTC |
| 708272 | AACCTGGCTCAGATCTGAAC | GCCAGGTATAGGGATTTGTG |
| 1864163 | TTAGAGGGGCTGTTGGAGAG | AGAGCCTGACACCTTCCCTA |
| 4369653 | AGTATCTTGACTTTATTTGG | GAAAAAAATATATGATAAAGG |
| 1864165 | TAAAAGGCTAGAAGTCCACC | AATTAATTCTCCCCTATAGC |
| 891141 | AGGCCCAGCCTGGGAAGTTT | TATCAGATGGTATCCACATG |
| 891143 | TCGTGCCCATCCTGTTAGTG | CACAAGCATGCCCTGTGTGG |
| 7205804 | AGGCAGCAAGCACCACAATG | ATGAACGGTGCCTGGTACAC |
| 5885 | GCAAAGAGATCAACGTCATC | ATGCAGACAGAAACGCACTCA |
| 1532625 | ACTGCTGTCTTCTGAGGCAT | CATCATGGCCGATTTTGTCC |
| 1532624 | TCTATAGACTTGCCCAACGC | TACTTTGGCAAATCTCTGCC |
| 289712 | ATTCGGCTTCTGTCATCCTC | GGGTCCAAAGCTTTTGTCAG |
| 7499892 | TGGCTGACTGGCCTGACCAC | CCCTCCATTCTGTACCACTTA |
| 5883 | TACTTCTGGTTCTCTGAGCG | TTGAACTCGTCTCCCATCAG |
| 289714 | ACACACACATACCACATGCC | TGATGGGAGACGAGTTCAAG |
| 158480 | TGAGTAGTTGGGACTATAGG | ACCCCTGTCTCTACAAAAAT |
| 289717 | CTCTGAGCCCAGAGTTGATC | CATTCCCTGCTCCATTTCCC |
| 4344729 | GCCTGAGTTCAGAAGGGAAG | GGCCGTTCTCCTGTTCTAAC |
| 289718 | GCCTGCCCAATATTGTGAGT | TCTTCCCTTCTGAACTCAGG |
| 289719 | TCTTCCCTTCTGAACTCAGG | GCCTGCCCAATATTGTGAGT |
| 2033254 | CACAACTCACAATATTGGGC | CAAAGGAACAGGACTCAGAC |
| 4784744 | ATCACATGCCCCAAGAAACC | CCGGCCCTTTCTTTCTTTTG |
| 291044 | AGGGCATCCCAGAACAGAAC | TGACTAGGTCAGGTCCCCTC |
| 8053613 | GAGTTCAGGGTAGGAATAGC | GCTGTGCAAATTAGGACTCT |
| 5881 | GACCCCTGTCTTCCACAGGT | ATCTTGGGCATCTTGAGGCA |
| 5880 | TTTCTCTCCCCAGGATATCG | CCAAGAGGCTTAAGAAGAGC |
| 7198026 | TGTTGGTGGGGAAATGTGGC | ACGAAGATTCTATCTAGGCA |
| 5882 | TCCAGGGAGGACTCACCATG | TGACTGCAGGAAGCTCTGG |
| 8045701 | TTGATACTTAGCGGTCCTGG | TAATATTCTGCAGGTAAACC |
| 289741 | TCTACCAGCTTGGCTCCCTC | AAGGGAGGGGCAGTAGGAGA |
| 1801706 | GTAGCAGAAGGCAAGCAC | AGGGAGGAGTTGGGAGCC |
| 289742 | ACTGGTGAGACAATCCCTTC | CCACTGGCATTAAAGTGCTG |
| 289743 | TGAAAAGAGGTGGACGGCAC | AGTCCTTCTTCTGTGGCTGG |
| 289746 | GAGGCTTACCAAAATGGGAC | AGAGCTTCTAGGCTTGGATG |
| 172337 | AGCTGGACTTTGAGGATGGC | AAGGAGAGGAGGGACTGGAG |
| 289747 | GTTGTTTAGGCCAAAAAGTC | CAATTACGGAAGTTACACTG |
| 1566439 | AAGCCCATGGCTTTTCAAGG | AGATCCTGGAGCCTCATTGT |
| 7205459 | GCAGAGGGGAAAGATCTTGG | AAGAGGGTATGTGTGTTGG |
| 289749 | TGAACTAAGGACCAGGCAAG | GCTCACATCCTTAGATTGCC |
| 289751 | AAATGATATGGAATTATGCG | TATCTTTCCAAATGTTTTAC |
| 8059220 | ACCTGCCATCATGAGTGCAT | TAGAGCAGGGCCTGGTGTGT |
| 8058353 | CCCTACAAGAAGGCTACATC | AGTGACTGCAGTGAAGAAGG |
| 289735 | CAAGGCATGCATGCTCCTTC | ATCGCCAGACCCTCAAGAAC |
| 289737 | GCAAGGAAGACTGATTCGTG | AGCCTCAAGTCACTGATGTC |
| 291042 | TGTCTCCCCATAACCAACAC | AAGAAACAGCTCCCAGTCTG |
| 1875236 | GAGCTGAGTGATTCTTGTCC | AATATGGTCAACTTGGTGGC |
| 821466 | AAGGAAGGAAGTGAGGGATG | GGAAAGCAAAGGAATGGCAG |
| 821465 | CACTGTTTACAATAGCCAAG | TGGAATTACAGGCGTGAGCT |
| 4275846 | GCAGCTGAGCAAAGATATGG | TGGACCTCTTCTATCATCAC |
| 289707 | CTCCTTCCTTCTGCTATCTC | ACCAAGATTCAGGCACTGAG |
| 821463 | CGTCTGCTGGGACACTGAAA | GCACCTGGAAGATTTATGGG |
| 289706 | TTAAGTCCTTTACCAAAGGG | TGAATGTGGCTTTACCAAGG |
| 1167741 | GCCCTATTTACGTGGATTTG | GCTCTGATTGTGTCTCTGTG |
| 2052880 | CATCAAGACTCCATGGAGAG | AGTCAAGAACCAGTCCCTAC |
| 1167742 | CCCTAGAAACTCCCTTATCC | TGAGTGGGGTACAGATGAAC |
| 1183256 | AGCTCAACTCCCCCCAAGTT | GAAGATAGGTGAGTTGAGGG |
| 1651665 | AAGTGAGAACAGGCTTCCTG | TCAAGGCTTTAGCTTGTCCC |
| 1651666 | TCTAACCTGGAACCCCTCAG | ATGAAGCCTGACACTTTGGG |
| 4784751 | CAAAGTGTCAGGCTTCATGG | AGCTGTGGGTGAGTGTCAAG |
| 1651667 | TCCCGACTTGTACACCTTGG | AGAAAATCAGCCAAAGGCTC |
| 8052091 | AGTCTTGCAGTCCAGGATG | TTCCCTGATGGACAGATGGC |
| 1684574 | TCTTGCAGTCCAGGATGCAG | GGCCATTTAGTGCTTGGAGC |
| 1684575 | TTTCCAGTCCCTGCATGTG | GAGGGGTTACCCAGAACCTT |
| 1672865 | AAAGGTTCTGGGTAACCCCT | CAGGCCTGAAAAAGCAAAGG |
| 821470 | CCTCTTTCTGTAATATCTGG | CTGGGCATTGCAGAACTGAA |
| 1549869 | CTGCAATAGTACACGTGGTG | AGATGTTGCAAAGGAGGTGC |
| 291040 | TACACTTGCCCAAAGTCCAC | TCTCTCTGTCAATCATGGGC |
| 289754 | ACTGCTTAGGTTGGCAAAGG | ATGCTTCCTTCCACAGGGAC |

TABLE 10

| | | |
|---|---|---|
| dbSNP rs# | Extend Primer | Term Mix |

| | | |
|---|---|---|
| 7500979 | TCAACCAATAGAAAAGGC | ACG |
| 2217332 | AACCTCTGGTCCTCTGGA | ACG |
| 8044804 | TAAGCCTTGGTATGATAC | ACG |
| 2270835 | GCCGTAAATTCCATTCTTC | ACG |
| 2133783 | CTGAACTTTACAGGTAACA | ACG |
| 247609 | AAACTATGTATCAGACAAAAGCAC | ACG |
| 952440 | CCTGGAAGGCAGCTGTGG | ACG |
| 881598 | CAGCCTGGGTGACAGAGC | ACT |
| 2291955 | GACTTTTCTAGGAAAGACTTA | ACG |
| 2518054 | TACCGGCACAAACAGTC | ACG |
| 866038 | CCCATCTCAAAAACAAAAAC | ACT |
| 1436425 | GGTGTGTGGCCCATGAT | ACG |
| 173537 | CATCACACCTGTCTGCCATC | ACT |
| 247611 | CTCCATGATTGGAGACTGACA | ACT |
| 166017 | CTACTGGCAGGGAACAT | ACT |
| 173538 | TTACAGGCGTAAGCCAC | ACG |
| 193694 | CTTAAAATCTACTCCCATACAT | ACT |
| 7205692 | CAAAAGAGTTAGGGGAG | ACG |
| 8048746 | CATGCCAAAATCTCGCC | ACG |
| 247618 | TTGGGGCCCCATGTAAA | ACG |
| 183130 | CTTCTGTGCAGGAGAAT | ACG |
| 6499863 | TTGAGGAGCAGTGGTCA | ACG |
| 4783961 | GGGTCCTGCCCTAGTCC | ACG |
| 3816117 | GAGGGAGATGGGCTGAG | ACG |
| 711752 | CAATGCAGCTAGGACCTTCT | ACG |
| 708272 | CTGGCTCAGATCTGAACCCTAACT | ACG |
| 1864163 | GGCTGTTGGAGAGGTTGATA | ACG |
| 4369653 | CAGGTGCTTTTACAAACAA | ACG |
| 1864165 | GTCCACCATGGCCCTCC | ACG |
| 891141 | CTGGGAAGTTTGCAGGG | ACT |
| 891143 | AGTGTGTCCACGGCTCC | ACG |
| 7205804 | CCTGTGCATCCATGGAG | ACG |
| 5885 | CATCTCTAACATCATGGC | ACG |
| 1532625 | TGAGGCATGCAGACAGAAAC | ACT |
| 1532624 | CAACGCCACACAGCTTGTGA | ACT |
| 289712 | CAGTGGATTGTGGCCCCC | ACG |
| 7499892 | AGCCCGTTGGCCTGAAC | ACG |
| 5883 | GTTCTCTGAGCGAGTCTT | ACG |
| 289714 | CATACCACATGCCATCTGGAT | ACG |
| 158480 | GGGACTATAGGTATGCAC | ACG |
| 289717 | TTCCAGCCCCCTACAAGTCACG | ACG |
| 4344729 | CAGAAGGGAAGAGGGAC | ACG |
| 289718 | TATGTGCAAGGAGAGAG | ACT |
| 289719 | CTGTGATGCCTCTCTCC | ACG |
| 2033254 | GGCAGGCCCCTGACCGGC | ACG |
| 4784744 | CCAAGAAACCACTGAAC | ACG |
| 291044 | AGTATTTAAAGGAGAGACACAC | ACG |
| 8053613 | GATGAGAAACTGAGGCC | ACG |
| 5881 | TTCCACAGGTTGTCGGC | ACT |
| 5880 | ATATCGTGACTACCGTCCAG | ACT |
| 7198026 | GAAATGTGGCCCCTTTC | ACG |
| 5882 | CAGAGCAGCTCCGAGTCC | ACT |
| 8045701 | CCTCTCCCTGCTGGTGG | ACG |
| 289741 | TGGGAGTCAGCCCAGCTC | ACT |
| 1801706 | CCTGGTGTCTCCTCCAGC | ACT |
| 289742 | GCCACAGAAGAAGGACTCC | ACT |
| 289743 | TGAGACAATCCCTTCCCCC | ACG |
| 289746 | TACCAAAATGGGACTGACCTC | ACG |
| 172337 | TTTGAGGATGGCCCCTGACACG | ACG |
| 289747 | TAGGCCAAAAAGTCTAAATTGC | ACT |
| 1566439 | CCTTAGAGAAACGGAAGGTG | ACT |
| 7205459 | ACTTTATTCCTGGATCAGA | ACG |
| 289749 | CAGGCAAGCTAATGCAA | ACT |
| 289751 | TGGAATTATGCGTTAAAGG | ACT |
| 8059220 | TGCATTCAGCTTCTCCA | ACG |
| 8058353 | CTACATCTGCCTCTCTATC | ACG |
| 289735 | GAGGGACAGAAGGGACC | ACT |
| 289737 | CTTACTTGTGTGGTTTAGAAT | ACT |
| 291042 | CCCATAACCAACACTCAGCT | ACG |
| 1875236 | GATTCTTGTCCTCTGAGAGC | ACG |
| 821466 | GAGGACAGGAGTGGAGCC | ACT |
| 821465 | ACAGAGAAAATGTGGCA | ACT |
| 4275846 | GATATGGTTTCAAAAGCCT | ACG |
| 289707 | TGCCATTCTCTAGGCCCGTC | ACT |
| 821463 | TGGGACACTGAAACCAGGG | ACG |
| 289706 | ACCAAAGGGATTGACTT | ACG |
| 1167741 | GAACTCTGAGTTACATTCC | ACG |
| 2052880 | CTTCCCTGCCTATTTTATGTC | ACT |
| 1167742 | CTTATCCCCCAACTCACCTT | ACT |
| 1183256 | TCATCTGCCCCCAGTTT | ACT |
| 1651665 | AGGGGTTCCAGGTTAGACCCTT | ACT |
| 1651666 | AGGAAGCCTGTTCTCACTTTC | ACG |
| 4784751 | TCATGGGGAAGCCCTAA | ACG |
| 1651667 | CTTGTACACCTTGGACTTGAC | ACT |
| 8052091 | CAACAGCCAGCAGCCCC | ACG |
| 1684574 | GATGCAGGAAGCAGGGCC | ACT |
| 1684575 | CACCTGTCTGTGGACTGGA | ACT |
| 1672865 | GGGTAACCCCTCAGCCCAG | ACT |
| 821470 | TCTGGATCCCCAGTGCC | ACG |
| 1549669 | TAGTACACGTGGTGTAAGGG | ACT |
| 291040 | CCAAAGTCCACCAGCCTCTACT | ACT |
| 289754 | CAAAGGATTTTAACTTCCTCTAG | ACG |

Genetic Analysis

Allelotyping results are shown for female cases and controls in Table 11. The allele frequency for the A2 allele is noted in the fifth and sixth columns for control pools and case pools, respectively, where “AF” is allele frequency. Some SNPs do not have an allele frequency disclosed because of failed assays.

TABLE 11

| | Position in | | | | | | | Low BMD |
|---|---|---|---|---|---|---|---|---|
| dbSNP | SEQ ID | Chromosome | A1/A2 | | | | | Associated |
| rs# | NO: 1 | Position | Allele | High AF | Low AF | p-Value | OR | Allele |
| 7500979 | 205 | 56743155 | G/A | G = 0.71 | G = 0.72 | 0.6029 | 1.07 | G |
| | | | | A = 0.29 | A = 0.28 | | | |
| 2217332 | 1595 | 56744545 | G/A | G = 0.85 | G = 0.84 | 0.5390 | 0.90 | A |
| | | | | A = 0.15 | A = 0.16 | | | |
| 8044804 | 2650 | 56745600 | C/T | C = 0.47 | C = | | | |
| | | | | T = 0.53 | T = | | | |
| 2270835 | 5496 | 56748446 | C/T | C = 0.00 | C = 0.00 | | | |
| | | | | T = 1.00 | T = 1.00 | | | |
| 2133783 | 5782 | 56748732 | G/A | G = 0.41 | G = 0.44 | 0.3412 | 1.14 | G |
| | | | | A = 0.59 | A = 0.56 | | | |
| 247609 | 5908 | 56748858 | C/T | C = 0.70 | C = 0.70 | 0.8844 | 1.02 | C |
| | | | | T = 0.30 | T = 0.30 | | | |
| 952440 | 7552 | 56750502 | G/A | G = 0.31 | G = 0.30 | 0.8410 | 0.97 | A |
| | | | | A = 0.69 | A = 0.70 | | | |
| 881598 | 9191 | 56752141 | T/C | T = 0.23 | T = 0.25 | 0.4703 | 1.11 | T |
| | | | | C = 0.77 | C = 0.75 | | | |
| 2291955 | 10127 | 56753077 | G/A | G = 0.00 | G = 0.00 | | | |
| | | | | A = 1.00 | A = 1.00 | | | |
| 2518054 | 10345 | 56753295 | G/A | G = 0.87 | G = 0.91 | 0.0835 | 1.47 | G |
| | | | | A = 0.13 | A = 0.09 | | | |
| 866038 | 10399 | 56753349 | T/C | T = 0.49 | T = 0.51 | 0.5835 | 1.07 | T |
| | | | | C = 0.51 | C = 0.49 | | | |
| 1436425 | 12028 | 56754978 | G/A | G = 0.39 | G = 0.38 | 0.7660 | 0.96 | A |
| | | | | A = 0.61 | A = 0.62 | | | |
| 173537 | 13355 | 56756305 | A/G | A = 0.80 | A = 0.81 | 0.8011 | 1.04 | A |
| | | | | G = 0.20 | G = 0.19 | | | |
| 247611 | 13687 | 56756637 | A/G | A = 0.38 | A = 0.39 | 0.7529 | 1.04 | A |
| | | | | G = 0.62 | G = 0.61 | | | |
| 166017 | 14328 | 56757278 | T/C | T = 0.29 | T = 0.43 | 0.00001 | 1.80 | T |
| | | | | C = 0.71 | C = 0.57 | | | |
| 173538 | 14746 | 56757696 | C/T | C = 0.68 | C = 0.67 | 0.5702 | 0.93 | T |
| | | | | T = 0.32 | T = 0.33 | | | |
| 193694 | 14996 | 56757946 | T/C | T = 0.14 | T = 0.24 | 0.0015 | 2.02 | T |
| | | | | C = 0.86 | C = 0.76 | | | |
| 7205692 | 19361 | 56762311 | G/A | G = 0.01 | G = 0.01 | | | |
| | | | | A = 0.99 | A = 0.99 | | | |
| 8048746 | 21775 | 56764725 | G/A | G = 0.00 | G = 0.00 | | | |
| | | | | A = 1.00 | A = 1.00 | | | |
| 247618 | 23250 | 56766200 | G/A | G = 0.28 | G = 0.29 | 0.5339 | 1.08 | G |
| | | | | A = 0.72 | A = 0.71 | | | |
| 183130 | 23810 | 56766760 | C/T | C = 0.64 | C = 0.59 | 0.0720 | 0.80 | T |
| | | | | T = 0.36 | T = 0.41 | | | |
| 6499863 | 24464 | 56767414 | G/A | G = 0.83 | G = 0.84 | 0.5810 | 1.09 | G |
| | | | | A = 0.17 | A = 0.16 | | | |
| 4783961 | 27341 | 56770291 | G/A | G = 0.48 | G = 0.46 | 0.3803 | 0.90 | A |
| | | | | A = 0.52 | A = 0.54 | | | |
| 3816117 | 28605 | 56771555 | C/T | C = 0.56 | C = 0.55 | 0.8019 | 0.97 | T |
| | | | | T = 0.44 | T = 0.45 | | | |
| 711752 | 28658 | 56771608 | G/A | G = 0.59 | G = 0.58 | 0.6887 | 0.95 | A |
| | | | | A = 0.41 | A = 0.42 | | | |
| 708272 | 28735 | 56771685 | C/T | C = 0.62 | C = 0.62 | 0.9629 | 1.01 | C |
| | | | | T = 0.38 | T = 0.38 | | | |
| 1864163 | 29680 | 56772630 | G/A | G = 0.74 | G = 0.74 | 0.9938 | 1.00 | G |
| | | | | A = 0.26 | A = 0.26 | | | |
| 4369653 | 29998 | 56772948 | G/A | G = 0.37 | G = 0.37 | 0.8163 | 0.97 | G |
| | | | | A = 0.63 | A = 0.63 | | | |
| 1864165 | 32521 | 56775471 | C/T | C = 1.00 | C = 1.00 | | | |
| | | | | T = 0.00 | T = 0.00 | | | |
| 891141 | 36170 | 56779120 | T/G | T = 1.00 | T = 1.00 | | | |
| | | | | G = 0.00 | G = 0.00 | | | |
| 891143 | 36427 | 56779377 | C/T | C = 1.00 | C = 1.00 | | | |
| | | | | T = 0.00 | T = 0.00 | | | |
| 7205804 | 37336 | 56780286 | G/A | G = 0.72 | G = 0.78 | 0.0212 | 1.41 | G |
| | | | | A = 0.28 | A = 0.22 | | | |
| 5885 | 37718 | 56780668 | C/T | C = 1.00 | C = 1.00 | | | |
| | | | | T = 0.00 | T = 0.00 | | | |
| 1532625 | 37748 | 56780698 | A/G | A = 0.60 | A = 0.61 | 0.6329 | 1.06 | A |
| | | | | G = 0.40 | G = 0.39 | | | |
| 1532624 | 37926 | 56780876 | T/G | T = 0.64 | T = 0.64 | 0.9807 | 1.00 | T |
| | | | | G = 0.36 | G = 0.36 | | | |
| 289712 | 38752 | 56781702 | G/A | G = 0.66 | G = 0.67 | 0.6644 | 1.06 | G |
| | | | | A = 0.34 | A = 0.33 | | | |
| 7499892 | 39037 | 56781987 | C/T | C = 0.88 | C = 0.88 | 0.9632 | 1.01 | C |
| | | | | T = 0.12 | T = 0.12 | | | |
| 5883 | 39800 | 56782750 | C/T | C = 0.95 | C = 0.96 | 0.8994 | 1.04 | C |
| | | | | T = 0.05 | T = 0.04 | | | |
| 289714 | 39898 | 56782848 | C/T | C = 0.19 | C = 0.23 | 0.0972 | 1.28 | C |
| | | | | T = 0.81 | T = 0.77 | | | |
| 158480 | 40674 | 56783624 | C/T | C = 0.59 | C = 0.56 | 0.3315 | 0.86 | T |
| | | | | T = 0.41 | T = 0.44 | | | |
| 289717 | 41835 | 56784785 | C/T | C = 0.67 | C = 0.72 | 0.1148 | 1.25 | C |
| | | | | T = 0.33 | T = 0.28 | | | |
| 4344729 | 42325 | 56785275 | C/T | C = 1.00 | C = 1.00 | | | |
| | | | | T = 0.00 | T = 0.00 | | | |
| 289718 | 42379 | 56785329 | A/G | A = | A = 0.20 | | | |
| | | | | G = | G = 0.80 | | | |
| 289719 | 42388 | 56785338 | G/A | G = | G = 0.39 | | | |
| | | | | A = | A = 0.61 | | | |
| 2033254 | 42432 | 56785382 | C/T | C = 0.34 | C = 0.38 | 0.1947 | 1.17 | C |
| | | | | T = 0.66 | T = 0.62 | | | |
| 4784744 | 43632 | 56786582 | G/A | G = 0.63 | G = 0.64 | 0.5113 | 1.08 | G |
| | | | | A = 0.37 | A = 0.36 | | | |
| 291044 | 43899 | 56786849 | C/T | C = 0.65 | C = 0.65 | 0.9666 | 1.01 | C |
| | | | | T = 0.35 | T = 0.35 | | | |
| 8053613 | 44273 | 56787223 | C/T | C = 1.00 | C = 1.00 | | | |
| | | | | T = 0.00 | T = 0.00 | | | |
| 5881 | 44459 | 56787409 | A/G | A = 0.00 | A = 0.00 | | | |
| | | | | G = 1.00 | G = 1.00 | | | |
| 5880 | 47538 | 56790488 | C/G | C = 0.09 | C = 0.09 | 0.9339 | 1.02 | C |
| | | | | G = 0.91 | G = 0.91 | | | |
| 7198026 | 47692 | 56790642 | C/T | C = 0.00 | C = 0.00 | | | C |
| | | | | T = 1.00 | T = 1.00 | | | |
| 5882 | 48539 | 56791489 | A/G | A = 0.76 | A = 0.80 | 0.0958 | 1.27 | A |
| | | | | G = 0.24 | G = 0.20 | | | |
| 8045701 | 48749 | 56791699 | C/T | C = 0.00 | C = 0.00 | | | |
| | | | | T = 1.00 | T = 1.00 | | | |
| 289741 | 49921 | 56792871 | A/G | A = 0.85 | A = 0.86 | 0.5974 | 1.09 | A |
| | | | | G = 0.15 | G = 0.14 | | | |
| 1801706 | 50109 | 56793059 | A/G | A = 0.31 | A = 0.23 | 0.0025 | 0.67 | G |
| | | | | G = 0.69 | G = 0.77 | | | |
| 289742 | 50209 | 56793159 | C/G | C = 0.91 | C = 0.92 | 0.6298 | 1.12 | C |
| | | | | G = 0.09 | G = 0.08 | | | |
| 289743 | 50243 | 56793193 | C/T | C = 0.27 | C = 0.24 | 0.1869 | 0.84 | T |
| | | | | T = 0.73 | T = 0.76 | | | |
| 289746 | 52652 | 56795602 | G/A | G = 0.72 | G = 0.72 | 0.8916 | 0.98 | G |
| | | | | A = 0.28 | A = 0.28 | | | |
| 172337 | 55195 | 56798145 | C/T | C = 0.94 | C = 0.91 | 0.0886 | 0.67 | T |
| | | | | T = 0.06 | T = 0.09 | | | |
| 289747 | 56385 | 56799335 | A/G | A = 0.49 | A = 0.54 | 0.0981 | 1.22 | A |
| | | | | G = 0.51 | G = 0.46 | | | |
| 1566439 | 57109 | 56800059 | A/G | A = 0.66 | A = 0.65 | 0.8578 | 0.98 | G |
| | | | | G = 0.34 | G = 0.35 | | | |
| 7205459 | 57618 | 56800568 | C/T | C = 0.16 | C = 0.10 | 0.0035 | 0.59 | T |
| | | | | T = 0.84 | T = 0.90 | | | |
| 289749 | 58741 | 56801691 | T/C | T = | T = 0.42 | | | |
| | | | | C = | C = 0.58 | | | |
| 289751 | 59222 | 56802172 | T/C | T = 0.98 | T = 0.94 | | | |
| | | | | C = 0.02 | C = 0.06 | | | |
| 8059220 | 60771 | 56803721 | C/T | C = 1.00 | C = 1.00 | | | |
| | | | | T = 0.00 | T = 0.00 | | | |
| 8058353 | 60962 | 56803912 | G/A | G = 0.98 | G = 0.95 | | | |
| | | | | A = 0.02 | A = 0.05 | | | |
| 289735 | 62009 | 56804959 | A/G | A = | A = 0.39 | | | |
| | | | | G = | G = 0.61 | | | |
| 289737 | 64589 | 56807539 | T/G | T = | T = 0.28 | | | |
| | | | | G = | G = 0.72 | | | |
| 291042 | 66054 | 56809004 | G/A | G = 0.80 | G = 0.82 | 0.4548 | 1.12 | G |
| | | | | A = 0.20 | A = 0.18 | | | |
| 1875236 | 66143 | 56809093 | C/T | C = 0.91 | C = 0.90 | 0.3106 | 0.80 | T |
| | | | | T = 0.09 | T = 0.10 | | | |
| 821466 | 67822 | 56810772 | A/G | A = 0.77 | A = 0.76 | 0.6743 | 0.94 | G |
| | | | | G = 0.23 | G = 0.24 | | | |
| 821465 | 68805 | 56811755 | C/G | C = 0.93 | C = 0.87 | 0.0041 | 0.54 | G |
| | | | | G = 0.07 | G = 0.13 | | | |
| 4275846 | 70075 | 56813025 | C/T | C = 1.00 | C = 1.00 | | | |
| | | | | T = 0.00 | T = 0.00 | | | |
| 289707 | 70350 | 56813300 | A/G | A = 0.44 | A = 0.46 | 0.6117 | 1.07 | A |
| | | | | G = 0.56 | G = 0.54 | | | |
| 821463 | 71214 | 56814164 | G/A | G = 0.38 | G = 0.41 | 0.4281 | 1.10 | G |
| | | | | A = 0.62 | A = 0.59 | | | |
| 289706 | 79549 | 56822499 | C/T | C = 0.00 | C = 0.00 | | | |
| | | | | T = 1.00 | T = 1.00 | | | |
| 1167741 | 82760 | 56825710 | C/T | C = 0.45 | C = 0.46 | 0.7635 | 1.04 | C |
| | | | | T = 0.55 | T = 0.54 | | | |
| 2052880 | 86463 | 56829413 | A/C | A = 0.67 | A = 0.68 | 0.8378 | 1.03 | A |
| | | | | C = 0.33 | C = 0.32 | | | |
| 1167742 | 86533 | 56829483 | C/G | C = 0.70 | C = 0.69 | 0.7322 | 0.96 | G |
| | | | | G = 0.30 | G = 0.31 | | | |
| 1183256 | 87019 | 56829969 | A/G | A = 1.00 | A = 1.00 | | | |
| | | | | G = 0.00 | G = 0.00 | | | |
| 1651665 | 88910 | 56831860 | A/C | A = 0.30 | A = 0.35 | 0.1423 | 1.24 | A |
| | | | | C = 0.70 | C = 0.65 | | | |
| 1651666 | 88955 | 56831905 | C/T | C = 0.71 | C = 0.67 | 0.1294 | 0.81 | T |
| | | | | T = 0.29 | T = 0.33 | | | |
| 4784751 | 89021 | 56831971 | C/T | C = 0.70 | C = 0.73 | 0.1971 | 1.18 | C |
| | | | | T = 0.30 | T = 0.27 | | | |
| 1651667 | 89056 | 56832006 | A/G | A = 0.44 | A = 0.47 | 0.2902 | 1.13 | A |
| | | | | G = 0.56 | G = 0.53 | | | |
| 8052091 | 89863 | 56832813 | G/A | G = 1.00 | G = 1.00 | | | |
| | | | | A = 0.00 | A = 0.00 | | | |
| 1684574 | 89879 | 56832829 | T/C | T = 0.01 | T = | | | |
| | | | | C = 0.99 | C = | | | |
| 1684575 | 90066 | 56833016 | T/G | T = 0.65 | T = 0.67 | 0.3473 | 1.12 | T |
| | | | | G = 0.35 | G = 0.33 | | | |
| 1672865 | 90101 | 56833051 | A/G | A = 0.49 | A = 0.50 | 0.6959 | 1.05 | A |
| | | | | G = 0.51 | G = 0.50 | | | |
| 821470 | 91029 | 56833979 | G/A | G = 0.37 | G = 0.38 | 0.8340 | 1.03 | G |
| | | | | A = 0.63 | A = 0.62 | | | |
| 1549669 | 91434 | 56834384 | T/G | T = 0.51 | T = 0.50 | 0.9075 | 0.99 | G |
| | | | | G = 0.49 | G = 0.50 | | | |
| 291040 | 93636 | 56836586 | T/C | T = 0.76 | T = 0.74 | 0.4056 | 0.89 | C |
| | | | | C = 0.24 | C = 0.26 | | | |
| 289754 | 98003 | 56840953 | C/T | C = 0.70 | C = 0.69 | 0.6508 | 0.94 | T |
| | | | | T = 0.30 | T = 0.31 | | | |

Allelotyping results were considered particularly significant with a calculated p-value of less than or equal to 0.05 for allelotype results. These values are indicated in bold. The allelotyping p-values were plotted in FIG. 1. The position of each SNP on the chromosome is presented on the x-axis. The y-axis gives the negative logarithm (base 10) of the p-value comparing the estimated allele in the case group to that of the control group. The minor allele frequency of the control group for each SNP designated by an X or other symbol on the graphs in FIG. 1 can be determined by consulting Table 11. For example, the left-most X on the left graph is at position 56743155. By proceeding down the table from top to bottom and across the graphs from left to right the allele frequency associated with each symbol shown can be determined.

To aid the interpretation, multiple lines have been added to the graph. The broken horizontal lines are drawn at two common significance levels, 0.05 and 0.01. The vertical broken lines are drawn very 20 kb to assist in the interpretation of distances between SNPs. Two other lines are drawn to expose linear trends in the association of SNPs to the disease. The light gray line (or generally bottom-most curve) is a nonlinear smoother through the data points on the graph using a local polynomial regression method (W. S. Cleveland, E. Grosse and W. M. Shyu (1992) Local regression models. Chapter 8 of Statistical Models in S eds J. M. Chambers and T. J. Hastie, Wadsworth & Brooks/Cole). The black line provides a local test for excess statistical significance to identify regions of association. This was created by use of a 10 kb sliding window with 1 kb step sizes. Within each window, a chi-square goodness of fit test was applied to compare the proportion of SNPs that were significant at a test wise level of 0.01, to the proportion that would be expected by chance alone (0.05 for the methods used here). Resulting p-values that were less than 10⁻⁸ were truncated at that value.

Finally, the exons and introns of the genes in the covered region are plotted below each graph at the appropriate chromosomal positions. The gene boundary is indicated by the broken horizontal line. The exon positions are shown as thick, unbroken bars. An arrow is place at the 3′ end of each gene to show the direction of transcription.

### Example 4

### PROL4 Proximal SNPs

It has been discovered that a polymorphic variation (rs1047699) in a gene encoding PROL4 is associated with the occurrence of low BMD (see Examples 1 and 2). One hundred twenty-five additional allelic variants proximal to rs1047699 were identified and subsequently allelotyped in low BMD case and high BMD control sample sets as described in Examples 1 and 2. The polymorphic variants are set forth in Table 12. The chromosome position provided in column four of Table 12 is based on Genome “Build 34” of NCBI's GenBank.

TABLE 12

| | Position in | | | | | |
|---|---|---|---|---|---|---|
| | SEQ ID | | Chromosome | Alleles | | |
| dbSNP | NO: 2 | Chromosome | Position | (A1/A2) | genome_letter | deduced_iupac |
| 523051 | 229 | 12 | 10842129 | t/c | g | R |
| 693620 | 368 | 12 | 10842268 | t/c | a | R |
| 2588349 | 583 | 12 | 10842483 | g/a | g | R |
| 2588350 | 2424 | 12 | 10844324 | t/c | t | Y |
| 619381 | 3625 | 12 | 10845525 | c/t | c | Y |
| 3759252 | 3709 | 12 | 10845609 | c/a | g | K |
| 3759251 | 3750 | 12 | 10845650 | t/a | t | W |
| 2418107 | 4724 | 12 | 10846624 | g/c | g | S |
| 7303054 | 4781 | 12 | 10846681 | t/c | c | Y |
| 1838345 | 6161 | 12 | 10848061 | a/g | g | R |
| 620878 | 7097 | 12 | 10848997 | t/g | g | K |
| 2537817 | 8025 | 12 | 10849925 | t/c | t | Y |
| 1548803 | 8398 | 12 | 10850298 | c/t | c | Y |
| 667123 | 10144 | 12 | 10852044 | g/a | a | R |
| 1838346 | 10384 | 12 | 10852284 | a/g | g | R |
| 2159903 | 11116 | 12 | 10853016 | a/g | g | R |
| 3944035 | 11132 | 12 | 10853032 | a/g | g | R |
| 3741845 | 11482 | 12 | 10853382 | t/c | a | R |
| 2110096 | 14544 | 12 | 10856444 | c/t | c | Y |
| 759055 | 15688 | 12 | 10857588 | t/a | t | W |
| 589377 | 17311 | 12 | 10859211 | g/c | g | S |
| 7960194 | 17831 | 12 | 10859731 | g/t | t | K |
| 7978242 | 20012 | 12 | 10861912 | a/g | g | R |
| 601051 | 21997 | 12 | 10863897 | g/a | g | R |
| 4262797 | 22861 | 12 | 10864761 | a/g | a | R |
| 2215714 | 23470 | 12 | 10865370 | a/g | g | R |
| 1373434 | 23515 | 12 | 10865415 | g/a | g | R |
| 2215715 | 23863 | 12 | 10865763 | g/a | g | R |
| 612456 | 24108 | 12 | 10866008 | c/a | c | M |
| 612808 | 24138 | 12 | 10866038 | t/c | c | Y |
| 689118 | 26469 | 12 | 10868369 | t/c | t | Y |
| 597468 | 27769 | 12 | 10869669 | a/g | g | R |
| 592864 | 29683 | 12 | 10871583 | t/c | t | Y |
| 640372 | 30491 | 12 | 10872391 | a/c | c | M |
| 7966559 | 30745 | 12 | 10872645 | a/t | a | W |
| 654834 | 31429 | 12 | 10873329 | t/c | t | Y |
| 4763216 | 31779 | 12 | 10873679 | c/g | c | S |
| 668521 | 32194 | 12 | 10874094 | a/c | c | M |
| 669503 | 32441 | 12 | 10874341 | t/c | c | Y |
| 3906864 | 32454 | 12 | 10874354 | t/c | g | R |
| 3906863 | 32459 | 12 | 10874359 | c/t | a | R |
| 7957888 | 35151 | 12 | 10877051 | a/t | t | W |
| 9300230 | 35362 | 12 | 10877262 | a/t | t | W |
| 7306214 | 35630 | 12 | 10877530 | g/c | g | S |
| 763839 | 36930 | 12 | 10878830 | g/c | g | S |
| 2418105 | 37490 | 12 | 10879390 | g/a | g | R |
| 666841 | 38432 | 12 | 10880332 | c/t | c | Y |
| 3851578 | 38688 | 12 | 10880588 | a/g | g | R |
| 7138797 | 39524 | 12 | 10881424 | t/c | c | Y |
| 7295252 | 41580 | 12 | 10883480 | a/c | a | M |
| 2418106 | 42531 | 12 | 10884431 | t/c | t | Y |
| 7299578 | 42665 | 12 | 10884565 | a/g | a | R |
| 621112 | 43038 | 12 | 10884938 | a/g | a | R |
| 3863320 | 44183 | 12 | 10886083 | g/a | g | R |
| 1373432 | 46271 | 12 | 10888171 | a/t | t | W |
| 1047699 | 49075 | 12 | 10890975 | c/t | t | Y |
| 1063193 | 49147 | 12 | 10891047 | c/t | c | Y |
| 2232959 | 49180 | 12 | 10891080 | c/g | g | S |
| 2227296 | 50301 | 12 | 10892201 | a/g | a | R |
| 1548804 | 50773 | 12 | 10892673 | g/a | g | R |
| 2232958 | 51243 | 12 | 10893143 | g/a | c | Y |
| 2232957 | 51530 | 12 | 10893430 | t/c | a | R |
| 2232956 | 52107 | 12 | 10894007 | a/g | c | Y |
| 1972571 | 52821 | 12 | 10894721 | a/g | a | R |
| 3759250 | 53341 | 12 | 10895241 | t/c | a | R |
| 3759249 | 53376 | 12 | 10895276 | c/g | g | S |
| 1541525 | 54047 | 12 | 10895947 | c/t | t | Y |
| 2098248 | 54392 | 12 | 10896292 | t/c | t | Y |
| 2900550 | 54482 | 12 | 10896382 | t/a | a | W |
| 7302130 | 55468 | 12 | 10897368 | c/a | a | M |
| 4763583 | 56990 | 12 | 10898890 | a/g | a | R |
| 4360778 | 57712 | 12 | 10899612 | a/t | a | W |
| 1607695 | 59667 | 12 | 10901567 | c/t | a | R |
| 1607694 | 59684 | 12 | 10901584 | t/c | g | R |
| 2192139 | 62043 | 12 | 10903943 | a/c | a | M |
| 7978300 | 63293 | 12 | 10905193 | t/c | t | Y |
| 7397871 | 63485 | 12 | 10905385 | g/t | g | K |
| 4763217 | 63778 | 12 | 10905678 | c/t | t | Y |
| 2159900 | 64222 | 12 | 10906122 | a/g | a | R |
| 10772370 | 65722 | 12 | 10907622 | a/g | a | R |
| 7398682 | 66315 | 12 | 10908215 | c/t | c | Y |
| 2900551 | 66829 | 12 | 10908729 | a/g | a | R |
| 2900552 | 66966 | 12 | 10908866 | c/t | c | Y |
| 2418214 | 66971 | 12 | 10908871 | a/c | c | M |
| 2418215 | 67013 | 12 | 10908913 | t/c | c | Y |
| 965243 | 70375 | 12 | 10912275 | a/t | t | W |
| 1117548 | 74118 | 12 | 10916018 | t/a | t | W |
| 1520225 | 75224 | 12 | 10917124 | c/t | c | Y |
| 1520226 | 75236 | 12 | 10917136 | t/c | t | Y |
| 1520227 | 75246 | 12 | 10917146 | g/c | g | S |
| 971919 | 75812 | 12 | 10917712 | c/a | a | M |
| 2159901 | 78968 | 12 | 10920868 | c/t | t | Y |
| 2159902 | 78998 | 12 | 10920898 | t/c | t | Y |
| 2110099 | 79328 | 12 | 10921228 | a/c | c | M |
| 7314847 | 80922 | 12 | 10922822 | t/c | t | Y |
| 7296003 | 81055 | 12 | 10922955 | t/c | c | Y |
| 4281556 | 81412 | 12 | 10923312 | g/a | a | R |
| 4763219 | 81785 | 12 | 10923685 | a/g | g | R |
| 3851579 | 82079 | 12 | 10923979 | g/a | a | R |
| 3851580 | 82087 | 12 | 10923987 | g/a | g | R |
| 1049119 | 82958 | 12 | 10924858 | a/g | c | Y |
| 2298866 | 83351 | 12 | 10925251 | t/g | a | M |
| 2298865 | 83442 | 12 | 10925342 | c/t | a | R |
| 2298864 | 83472 | 12 | 10925372 | a/c | t | K |
| 2298863 | 83966 | 12 | 10925866 | a/g | t | Y |
| 3180393 | 84414 | 12 | 10926314 | t/c | t | Y |
| 2070837 | 86563 | 12 | 10928463 | c/g | c | S |
| 7956204 | 86788 | 12 | 10928688 | g/a | g | R |
| 2418216 | 86796 | 12 | 10928696 | t/g | t | K |
| 3741844 | 87634 | 12 | 10929534 | g/a | c | Y |
| 4262798 | 88530 | 12 | 10930430 | g/a | a | R |
| 2418217 | 89202 | 12 | 10931102 | a/g | a | R |
| 2418218 | 89632 | 12 | 10931532 | t/c | t | Y |
| 7137492 | 89697 | 12 | 10931597 | t/c | t | Y |
| 2110100 | 89723 | 12 | 10931623 | g/a | g | R |
| 1013312 | 91063 | 12 | 10932963 | c/g | c | S |
| 4579993 | 91335 | 12 | 10933235 | c/g | g | S |
| 1013313 | 91504 | 12 | 10933404 | t/c | t | Y |
| 7397106 | 91619 | 12 | 10933519 | c/t | t | Y |
| 2215716 | 93715 | 12 | 10935615 | a/g | g | R |
| 2192140 | 93945 | 12 | 10935845 | t/c | c | Y |
| 4763589 | 94235 | 12 | 10936135 | t/c | t | Y |
| 1468697 | 95851 | 12 | 10937751 | t/c | c | Y |
| 2070837 | 130860 | 12 | 10972760 | c/g | c | S |
| 3180393 | 132815 | 12 | 10974715 | t/c | a | R |
| 2298865 | 133778 | 12 | 10975678 | c/t | t | Y |

Assay for Verifying and Allelotyping SNPs

The methods used to verify and allelotype the proximal SNPs of Table 12 are the same methods described in Examples 1 and 2 herein. The primers and probes used in these assays are provided in Table 13 and Table 14, respectively.

TABLE 13

| | | |
|---|---|---|
| dbSNP | Forward | Reverse |
| rs# | PCR primer | PCR primer |

| | | |
|---|---|---|
| 523051 | CTCTGCTCAGAGCATAGATG | TTCCATGCATTTTAACCCCG |
| 693820 | GTAGGTCAATAAAAGAGGAGG | TTAGGCCGAGATTTTCAGAG |
| 2588349 | GTTCAAGAGTTTGATGTCAAG | CAGGGCATCTAAATTGAACG |
| 2588350 | TGTATCTGGGACATACCATC | GTTAGTACAGGGTTGATCAC |
| 619381 | AAAGCATAGTTTCTCTTCAG | GCATCTCTAAAGGTGATTTGG |
| 3759252 | GCTGTGATTTTTGGTGAGTC | TATTGTTCCCCAGTATTAGG |
| 3759251 | TTGTCCTTTCTCATTGCCAC | ATTAGAGCTATGGACTCACC |
| 2418107 | AGGTAAATGGGCATACAGTG | TGAAGACTGGAGCTATCTTC |
| 7303054 | AGGGAATTGGCCCAATTTGC | CCATTTACCTACTCAGCAGG |
| 1838345 | CCCAGGTTCAAGTGATTCTC | AATTAGCCAGGCATGGTTGC |
| 620878 | CCAGACCAAACGTTTCAATAA | AAGAAGAAACGAATGTTTAC |
| 2537817 | GTCAGAATGCTGACATGTAG | GGGGAAAAATGAGAATAGAC |
| 1548803 | ATGGGACAATTGCAAACAGG | ACTGAAATGTTCCATGTGAG |
| 667123 | TAAATACAAGAAGCCCTAGG | AGGGAAAGTACAACTTAGCC |
| 1838346 | GTTAGAAGGCTAGAGAGAAC | TTCATGAGTACTCTAGCTGG |
| 2159903 | AGTCAGGATACTCTTTAGGG | GGGAAGCTTTTCTGAAGATG |
| 3944035 | GGGTCTCTATGGAACAAAAG | TGAGGGAAGCTTTTCTGAAG |
| 3741845 | TGCTTGGTGTGTCTAACTAG | CAAACAGTTAACCCTGAACC |
| 2110096 | ATCAAGAACAGGATACTGCG | GGGTAGGAGATAAAGTCAGC |
| 759055 | CCCCTATCTTTTTGTTGGATG | GCTCTGTTTTCATGAGGTTC |
| 589377 | TTCACTGTTCAGATTGCTGG | ACTCCAGTTGTAGGTAGAAG |
| 7960194 | TGCTATCTTCCATGGAAGAA | TTTTTTTCCCTGTATGCCTC |
| 7978242 | GACGCTAAGCATCATTAGTC | TAGCCATTCTAGTGGATGTG |
| 601051 | TTTTTACGTCTGTGTGAACC | CACAGAAACCGAAAACCAAC |
| 4262797 | ATTTTAGTGCACCCATCACC | GTCTTTAGAGACTCAAAAGGG |
| 2215714 | GAGTTAAACATCAGTCGTTGT | TTATTATATAACGTAGGAA |
| 1373434 | CGTTTAAGATGATAGATCTTC | CTACGCAATAGGCTATTTTC |
| 2215715 | ACATGGATGGAAAGGAAGAC | TATGAGCGAGAATATGTGGC |
| 612456 | TATCAACAAGCCAAGATCCC | CTTTAGCCTTTTGTTGGACG |
| 612808 | GGCTGAGTGTTAAGTATTCC | AAGGCAAACCCTTAATAGGC |
| 689118 | GAAATGTTCTGTCTTCAGGC | AACGTTTCTGTGAGTGCAAG |
| 597468 | TACATGTTGAGATCCCAGAC | TGCATACATTGCGAAGATTC |
| 592864 | AAATTGTGTTTCTTGTAAGC | AATGAACTAAATACTCCAAG |
| 640372 | ATCTGAAACTCTTTGAGCGC | TCCAAAATGTTCCAATGAGC |
| 7966559 | CCTAATTCTTTTCTATCGGTG | GAAGCTAGAAAGCTAAGAGTG |
| 654834 | CTGTCCTTTTTCTGGCTTTC | CAAACAAAATAGAGTCCAGCG |
| 4763216 | TTGTAACCATAGAAAGCAGG | TCCCTCTATTTTTCTACTAC |
| 668521 | TGCTGCTGATGTAACTGCAG | GTGCAATAGTAACTGGGCTC |
| 669503 | GTAATCCACTCATTCAGATC | TACCTAGAGGAAATAAGTGG |
| 3906864 | TTTCTAACATCCACCTTCTG | GGTGTGAATAAGTTGTAATCC |
| 3906863 | CAGGACTGACCTCTAAAATAG | ACATCCACTTTCTGTAGATC |
| 7957888 | ACCTTAACAAAGTTCTGTGG | GAGCCACGAAAAAAAAAATG |
| 9300230 | CCAGACAAGCTAAAATACAG | CAAATACACACAGACACATC |
| 7306214 | CTATAGCACAGTAGGGTGAC | TCTTCGTGTTGGGCACATTC |
| 763839 | TATTCATGTAACACCGAAGG | GGAACAGCAAACTGAAACAG |
| 2418105 | GGAGGCATTCCATTCTTTTC | GTGTATCCAAAGCTTTAGTG |
| 666841 | TAATCCCCAAGAGAGAGGAC | AACTAACACAGATGGCCACC |
| 3851578 | GTGTGTATTTGCCCTTCTGC | GGCATGTGATAGGAATGTGC |
| 7138797 | GTTCTGAAGTACATGTGCAG | TTAATACCTAGGTGATGGGC |
| 7295252 | CAAGTTTACAATCACAGCTG | GGTAAATGTATTTGACAGTTG |
| 2418106 | CAGCCCAGAATGGCTTTGAA | ACTAACGATGGGTGATGAGC |
| 7299578 | ATCTGCCCCATGATCCAATC | CCCCTGAAAACCTCATGTTG |
| 621112 | TATGTTGGGTCACAAACCTG | AGCAAGGCGTACAAAACAAC |
| 3863320 | GTTGCTCACAATACTGGAGG | TGTCTTCAGCAGAACTCATC |
| 1373432 | TAAGGCAGAGGGCTACTTAC | GCATTTTACAAGACTTAAATC |
| | | G |
| 1047699 | ATTACCAGAGTGGTTGCTCC | ATTTCCTTCTGTCAGCCTGC |
| 1063193 | AGGCTGACAGAAGGAAATCG | TCAGCAGAGACCACCAAAAC |
| 2232959 | TCAGCAGAGACCACCAAAAC | GGTGGTCGTTGCTGATTTTG |
| 2227296 | TGAGAAGAAGACACTGAAGG | ATCCCAATGATCCTCAATCC |
| 1548804 | CCCATTTTTCTTCCAATCAC | GGAGCAACCAGGAGTAAATA |
| 2232958 | TCTTCTCAACCTCCTTCCTC | ATGACTCTTCTAGGGCCTCA |
| 2232957 | TTTATAAACATGAGCAGAAC | CGCATTCAACTTTATGAGAGG |
| 2232956 | GCCAATTTTGCCTTACTAACC | CTAGCATTAACCAGAGATAC |
| 1972571 | ATCACCAGCTGCCACTTTTC | CCTCAAATTATCACCAGCCC |
| 3759250 | TTCTGCCTAGGTGGCTTATG | AATCATGTCTGCATGGCACG |
| 3759249 | CACACAGTGAATCATGTCTG | ATGGTAGACCATCAGGTAGG |
| 1541525 | TATCTGTAGACGTGCATCCC | CTGACACCTGTCCTCATAAG |
| 2098248 | GGGAGAATCATGGCTTGA | GTACTGCCCTTAGCAAATCC |
| 2900550 | TCAACAGTCTCCAGCTGATC | ACTCTGGCTGTTGTAAGGAG |
| 7302130 | TACACCTGTGAGCACTGACA | CCTTTTCTCAAGGTGTGTGC |
| 4763583 | AATGGTTTAGCACCATCCCC | GGGAGGTGACACACACTTTT |
| 4360778 | AGGCTGCAAGTTTTCCAAAC | GCCTAAGCAAAGAAATGAGC |
| 1607695 | TTCTTCTCACTGACCTATTC | ATCAAACAGAAACGCTGGAG |
| 1607694 | ATCAAACAGAAACGCTGGAG | CTCTTTTTTTGTCTGACTGGG |
| 2192139 | GGTCTTTTGTTTGCTAGGAC | GTAACCTGCCAAGATTGAAC |
| 7978300 | TCTGAGTCTTTTGTGGTACC | TGCAGATTCAATGCAATCCC |
| 7397871 | AGGAGCTGAAACGATGGTTC | CAAAATGGCATGACACTGGC |
| 4763217 | ACATTGTGTCTGTGCTTCTC | GCAGTCTCTTATTGTCTGAG |
| 2159900 | TTACAACAAGTGCTCAAGGG | GTGTCTTCTCTAACAGTGAG |
| 10772370 | GAATGACCATATGATCCAGC | TAGGTATCCCTTTGATGTCC |
| 7398682 | GACAGATACCCTAACTTGATC | GCAGTAAAGCTATCAATCCTG |
| 2900551 | GTACCTCAACACAAAAAGGC | GAGGAAATCTTTCACCTTTCC |
| 2900552 | GCTAGATATACTAGGCAAGAG | TGTCATCCGAAAAGATGAAC |
| 2418214 | TGTCATCCGAAAAGATGAAC | GCTAGATATACTAGGCAAGAG |
| 2418215 | CTATTCTGCAACTTTACCAG | TAGAAGGAAGTCAATTGTTC |
| 965243 | GGCTGTGTAGGTTTGTCCTC | TTCATCCCATTCCATCCAGC |
| 1117548 | GTGGTACATCACATTAACAG | TTCCTTCTTTGTGTAGTTCG |
| 1520225 | CGTGACTCTCTGTACAGCAT | CAGCACTAGGCTGTGAAAAG |
| 1520226 | TAGGGTAAAATGTGCACAGC | GCATTGTTAGTGGTTGCTCC |
| 1520227 | TAGGGTAAAATGTGCACAGC | GCATTGTTAGTGGTTGCTCC |
| 971919 | TTAGTGACCTTCATAGAACC | CAGGCATTCTTAGAAGACAG |
| 2159901 | CATATCTACTTGTGAACTGC | GAAGGTAAAACCACTGATGC |
| 2159902 | CCCCTATAAGAGCAAAATAC | GAACTTTTCAGAAAGCATCAG |
| 2110099 | ATTCCTTCTCAACCCACATC | ACATTACAGGAAGGCCTTTC |
| 7314847 | CCTCTGCATAGATACAGTAG | CTCTTATACACCCTGATTGG |
| 7296003 | TGGTTACTTAGGTTGATGCC | CTACTGTATCTATGCAGAGG |
| 4281556 | GGCCTTAGGGTTTCCATAAC | AAGCCTTGGAAGAGTGAGTG |
| 4763219 | AGGAGAATCGCTTGAACCTG | TGGAGTCTCATTCTGTCACC |
| 3851579 | AGGCTTTCAAACAAAATAGG | TTACGGGTCTGATAAGAAAG |
| 3851580 | AGGCTTTCAAACAAAATAGG | TTACGGGTCTGATAAGAAAG |
| 1049119 | GATAACAGTGTTTCAAATGC | GAAATTGCAAGCTGATTGTT |
| 2298866 | TAACCAATCCCTGTCACTGG | ACAAAGATGGGCACTGCAAC |
| 2298865 | CCTGAGTTCATTTAGATCTC | ACGGAACAACCAAACAATAG |
| 2298864 | CCAGTAAACACTGAAGAGATC | GATTTGTGTCTTACTCACTGG |
| 2298863 | AGCTCTTGAAGGCAATTCTG | TATGTCCTCATTGTCAACCC |
| 3180393 | AACAAGGTCCACCACCTCCT | TTGTGGCCTTCCTTGAGGAG |
| 2070837 | ACACACCCACACAAACTCAC | TGGAAAGACTGCTATTCTGC |
| 7956204 | TTTGGACAATTCTTGTAGCC | TGTGAGATGTGTGAGGACAG |
| 2418216 | CCCAGGCACACATATATGAG | GCCTGAGTGTAGTGAGATTC |
| 3741844 | GCTGATTGCTCTGTGATACC | AAGTGCAGCTGGTGATTCTG |
| 4262798 | TCTAGGTACCCAAGCTCCTG | AAGAGCTGAAAGGGACACTG |
| 2418217 | GCCTGGAACACTAAAGATGG | CTCACTGACTCTCAGAGAAG |
| 2418218 | AAATTAGTCAGTCATGGTGG | TTCAAGCAATTCTCCTGCCT |
| 7137492 | TCCTCATTTACTACAGTGAC | CCCATCTCTACTAAAAATAC |
| 2110100 | CTGGAATTACAAAGAGAAGAG | CCCATCTCTACTAAAAATAC |
| 1013312 | ATTGTGTTTGCCCACTTTCC | GTGGAACATCAAGAATGAAG |
| 4579993 | ATTTGTGCTCCTTTCTACTG | AGCAGAAAGAAGGAAATGAC |
| 1013313 | TTCCAAACACAGCAAAGAGC | TTGGTTGTATTCTGGTTGGG |
| 7397106 | TCTGAAACTCAGAATGCATG | ATACTCTAAACAATACAGGG |
| 2215716 | GTACTAGCAGTAGTCAGAGG | GACACCACTACTTGCACATG |
| 2192140 | CCTTGGGATTTAGAAATAGGG | GTAAGCAAATATCTCTGGAG |
| 4763589 | TTGGCAACTGTGTAACCTTG | TCAAACATACTGTTTGCTCAC |
| 1468697 | GAACACAATCGCAAGTTTAAG | GTTCTTCAAAATCTGCTTCC |
| 2070837 | ACACACCCACACAAACTCAC | TGGAAAGACTGCTATTCTGC |
| 3180393 | AACAAGGTCCACCACCTCCT | TTGTGGCCTTCCTTGAGGAG |
| 2298865 | CCTGAGTTCATTTAGATCTC | ACGGAACAACCAAACAATAG |

TABLE 14

| | | |
|---|---|---|
| dbSNP | Extend | Term |
| rs# | Primer | Mix |

| | | |
|---|---|---|
| 523051 | TCAGAGCATAGATGATGGCAA | ACT |
| 693620 | AGAGGAGGAAACCTTAATTTCT | ACT |
| 2588349 | AGAGTTTGATGTCAAGGAAATG | ACG |
| 2588350 | TACCATCAAAAGCACATCATTC | ACT |
| 619381 | GAATTTTCTTCCTTTTAGAATAGA | ACG |
| 3759252 | TGGTGAGTCCATAGCTCTAAT | CGT |
| 3759251 | CCAGCTACTTTATGCCAGAG | CGT |
| 2418107 | TGGCCAGGCCAAGGATCG | ACT |
| 7303054 | CAGCCTAAATTAACCGTATGTG | ACT |
| 1838345 | CCTGCCTGAGCCTGCCAA | ACT |
| 620878 | TTCAATAATATTATAGTGAGGATG | ACT |
| 2537817 | GACATGTAGAAAAATTGCCTGC | ACT |
| 1548803 | TTGCAAACAGGTTAAAGAGAGT | ACG |
| 667123 | AAGCCCTAGGGTATTGTGATT | ACG |
| 1838346 | AAGGCTAGAGAGAACATTCCA | ACT |
| 2159903 | AGGGGTCTCTATGGAACAAAA | ACT |
| 3944035 | AACAAAAGGCTTTCTTCTTCTAA | ACT |
| 3741845 | CAAAGGATAAAGGGAACCATC | ACT |
| 2110096 | CAGGATACTGCGATACTGTC | ACG |
| 759055 | GTAATCTTGTTTGTTGTATTTTTC | CGT |
| 589377 | CTGGTGTGTAGAAACACAACA | ACT |
| 7960194 | GCCCAGGGCTCAGGGAAG | CGT |
| 7978242 | GCAAACCAAAACCACAGTCAC | ACT |
| 601051 | CCCGCTTATAAGCAAGAGCA | ACG |
| 4262797 | TTATCCCTATCCTCCCTTCC | ACT |
| 2215714 | GCATCACTCTTTCTACGCAATA | ACT |
| 1373434 | TGTTTCAAAACATCACTATGTAC | ACG |
| 2215715 | GGAAAGGAAGACATTATGGTAC | ACG |
| 612456 | CCAAGATCCCTGAAAGGCAAA | CGT |
| 612808 | CTACTTTAGCCTTTTGTTGGAC | ACT |
| 689118 | GCTTCATACACACACACACAT | ACT |
| 597468 | CCCAGACTGTGTCATTCTTC | ACT |
| 592864 | GTGTTTCTTGTAAGCAGTATAC | ACT |
| 640372 | AAACTCTTTGAGCGCTGACAT | ACT |
| 7966559 | TCTATCGGTGATTTCTGCTGT | CGT |
| 654834 | CACAATTGGCTTTTAAAACTCC | ACT |
| 4763216 | CCATAGAAAGCAGGACTGGTT | ACT |
| 668521 | AAATTAGTCTAGAACTTGTGGAA | ACT |
| 669503 | CCACTCATTCAGATCTACAGAA | ACT |
| 3906864 | ACATCCACCTTCTGTAGATCT | ACT |
| 3906863 | GTGTGAATAAGTTGTAATCCAC | ACG |
| 7957888 | TGTGGAAATATTTGAAGACTCTT | CGT |
| 9300230 | ATACAGAGAAGTAGAGGACAAA | CGT |
| 7306214 | CACAGTAGGGTGACTACAATTA | ACT |
| 763839 | TAACACCGAAGGTTCCTCAG | ACT |
| 2418105 | CATTCTTTTCCAGTGTCAATCAA | ACG |
| 666841 | GGACTTGGCAGCATTATTTATTA | ACG |
| 3851578 | CAGCTAATTGTGCTCCCTCA | ACT |
| 7138797 | AAGTACATGTGCAGAATGTGC | ACT |
| 7295252 | ATCACAGCTGATAATGTCATAAT | ACT |
| 2418106 | GAATGGCTTTGAATATGACCCA | ACT |
| 7299578 | CAGGTGCTATCACAACATCG | ACT |
| 621112 | CACAAACCTGCCATTTTACTTT | ACT |
| 3863320 | AATACTGGAGGCTGGAAGAC | ACG |
| 1373432 | CAGAGGGCTACTTACAAGAATT | CGT |
| 1047699 | TCTTGCTGGTCTGTCCCTC | ACG |
| 1063193 | AGTTGACGGTGTCCTCGT | ACG |
| 2232959 | ATCACCGCCATCCTCCCC | ACT |
| 2227296 | GAAATTCTAGTGGAAAAAG | ACT |
| 1548804 | AAAATTTCAATATGTTGCAGGCAG | ACG |
| 2232958 | CCTCCCCTCCTGCTCTTC | ACG |
| 2232957 | GGACCTCACCACCTGAAAG | ACT |
| 2232956 | TGCCTTACTAACCTCCAGG | ACT |
| 1972571 | CTGCCACTTTTCATTACAGGC | ACT |
| 3759250 | TAGGTGGCTTATGGTAGACC | ACT |
| 3759249 | TCTGCATGGCACGCCCCT | ACT |
| 1541525 | CCCTCTGTGGTCTGTGCTAA | ACG |
| 2098248 | GAATCATGGCTTGACTCAGG | ACT |
| 2900550 | GCCCCTCACCACATCCCA | CGT |
| 7302130 | CCCACCCAGCAGGAAGAC | CGT |
| 4763583 | CCTGCGTACTATTCTTATGAC | ACT |
| 4360778 | TTTTACACTCTGCTTCCCTTTT | CGT |
| 1607695 | TTCTCACTGACCTATTCTATTTT | ACG |
| 1607694 | AGCTTCAAAAATAGAATAGGTCA | ACT |
| 2192139 | CTTTGATCTCATCACTTGTTATT | ACT |
| 7978300 | TTCTGTTTCTGTGAAGAATGTC | ACT |
| 7397871 | GCCAGGCTCACAGTCCAAA | CGT |
| 4763217 | TTGGACGTAGGACAAAGAACT | ACG |
| 2159900 | GCTCAAGGGAGTTCTACATCT | ACT |
| 10772370 | GACCATATGATCCAGCAATTTC | ACT |
| 7398682 | GCTTATAACAAAATATCATGTGTC | ACG |
| 2900551 | GCCATATATGACAAACCCACA | ACT |
| 2900552 | ATATACTAGGCAAGAGATAGAAA | ACG |
| 2418214 | CTTCCTTCTATTCAATTTCAATTT | ACT |
| 2418215 | CTATAAATAAGATCATGTCATCC | ACT |
| 965243 | CAGACCCATCAGAGAAGTAC | CGT |
| 1117548 | CCAAATAAGCATATGAATAGATG | CGT |
| 1520225 | CAGCATTGTTAGTGGTTGCTC | ACG |
| 1520226 | GCTGTGAAAAGCTATGTATGC | ACT |
| 1520227 | CAGCACTAGGCTGTGAAAAG | ACT |
| 971919 | ATAAGATAGGTCTGTTGGAAAG | CGT |
| 2159901 | TGATTTCTTCAGCCATTATCCA | ACG |
| 2159902 | TTAAGCTTGCATGAAAGAAGGT | ACT |
| 2110099 | TTATGTCAGCAGAAAAACAGAAA | ACT |
| 7314847 | GAGTTTTAGTTCTTTGAGAAATC | ACT |
| 7296003 | GGTTGATGCCATATCTTTGCT | ACT |
| 4281556 | GGTTTCCATAACAAGAATAACAAA | ACG |
| 4763219 | CCGATATCTTGCCACTGCAG | ACT |
| 3851579 | CAAAATAGGAACACACTGAGTAT | ACG |
| 3851580 | CTTTCAAACAAAATAGGAACACA | ACG |
| 1049119 | TGGCATCATGCTCTAACTTCA | ACT |
| 2298866 | CCTGTCACTGGATATTAAGGC | ACT |
| 2298865 | CATTTAGATCTCTTCAGTGTTTA | ACG |
| 2298864 | CTGAAGAGATCTAAATGAACTCA | ACT |
| 2298863 | CAATTCTGATTTTGAGAATCACT | ACT |
| 3180393 | GCCACAAGGACCACCCCA | ACT |
| 2070837 | CCACACAAACTCACATACACA | ACT |
| 7956204 | AGGCTGCCAGCACCTTTCT | ACG |
| 2418216 | GCACACATATATGAGAGAAAGG | ACT |
| 3741844 | GTGATACCTAGAAATTCCCTG | ACG |
| 4262798 | CGTCTCCCTTTCACCCACC | ACG |
| 2418217 | TGCCAGGAAAGATGAACATTG | ACT |
| 2418218 | CAGTCATGGTGGCGAGGG | ACT |
| 7137492 | GCCATGTGACTTAATTTCTTTAG | ACT |
| 2110100 | TCCTCATTTACTACAGTGACC | ACG |
| 1013312 | GCCCACTTTCCCCCTTTCT | ACT |
| 4579993 | ACTGTTATTTCTGGTTTCTGGT | ACT |
| 1013313 | ACAGCAAAGAGCATAAACCTAC | ACT |
| 7397106 | CTCAGAATGCATGAATAGTACA | ACG |
| 2215716 | CGTACTATTTCTTCAAGCTTCA | ACT |
| 2192140 | TAGGGTATCCCTCTTGGTCA | ACT |
| 4763589 | CTGTGTAACCTTGATCTTGGG | ACT |
| 1468697 | CAATCGCAAGTTTAAGGTAACA | ACT |
| 2070837 | CCACACAAACTCACATACACA | ACT |
| 3180393 | GCCACAAGGACCACCCCA | ACT |
| 2298865 | CATTTAGATCTCTTCAGTGTTTA | ACG |

Genetic Assay

Allelotyping results are shown for female cases and controls in Table 15. The allele frequency for A2 allele is noted in the fifth and sixth columns for control pools and case pools, respectively, where “AF” is allele frequency. Some SNPs do not have an allele frequency disclosed because of failed assays.

TABLE 15
Female Allelotyping Results

| | Position in | | | | | | | Low BMD |
|---|---|---|---|---|---|---|---|---|
| dbSNP | SEQ ID | Chromosome | A1/A2 | Controls (high | Cases (Low | | | Associated |
| rs# | NO: 2 | Position | Allele | BMD) AF | BMD) AF | p-Value | OR | Allele |
| 523051 | 229 | 10842129 | T/C | T = 0.00 | T = 0.00 | | | |
| | | | | C = 1.00 | C = 1.00 | | | |
| 693620 | 368 | 10842268 | T/C | T = 1.00 | T = 1.00 | | | |
| | | | | C = 0.00 | C = 0.00 | | | |
| 2588349 | 583 | 10842483 | G/A | G = 1.00 | G = 1.00 | | | |
| | | | | A = 0.00 | A = 0.00 | | | |
| 2588350 | 2424 | 10844324 | T/C | T = 0.34 | T = 0.28 | 0.0295 | 0.76 | C |
| | | | | C = 0.66 | C = 0.72 | | | |
| 619381 | 3625 | 10845525 | C/T | C = 0.86 | C = 0.91 | 0.0065 | 1.67 | C |
| | | | | T = 0.14 | T = 0.09 | | | |
| 3759252 | 3709 | 10845609 | C/A | C = 1.00 | C = 1.00 | | | |
| | | | | A = 0.00 | A = 0.00 | | | |
| 3759251 | 3750 | 10845650 | T/A | T = 0.01 | T = 0.01 | | | |
| | | | | A = 0.99 | A = 0.99 | | | |
| 2418107 | 4724 | 10846624 | G/C | G = 1.00 | G = 1.00 | | | |
| | | | | C = 0.00 | C = 0.00 | | | |
| 7303054 | 4781 | 10846681 | T/C | T = 0.00 | T = 0.00 | | | |
| | | | | C = 1.00 | C = 1.00 | | | |
| 1838345 | 6161 | 10848061 | A/G | A = | A = | | | |
| | | | | G = | G = | | | |
| 620878 | 7097 | 10848997 | T/G | T = 0.07 | T = 0.03 | 0.0098 | 0.37 | G |
| | | | | G = 0.93 | G = 0.97 | | | |
| 2537817 | 8025 | 10849925 | T/C | T = 0.19 | T = 0.16 | 0.4485 | 0.85 | C |
| | | | | C = 0.81 | C = 0.84 | | | |
| 1548803 | 8398 | 10850298 | C/T | C = 0.47 | C = 0.41 | 0.0564 | 0.78 | T |
| | | | | T = 0.53 | T = 0.59 | | | |
| 667123 | 10144 | 10852044 | G/A | G = 1.00 | G = 1.00 | | | |
| | | | | A = 0.00 | A = 0.00 | | | |
| 1838346 | 10384 | 10852284 | A/G | A = 0.76 | A = 0.80 | 0.1329 | 1.26 | A |
| | | | | G = 0.24 | G = 0.20 | | | |
| 2159903 | 11116 | 10853016 | A/G | A = 0.00 | A = 0.00 | | | |
| | | | | G = 1.00 | G = 1.00 | | | |
| 3944035 | 11132 | 10853032 | A/G | A = 0.00 | A = 0.00 | | | |
| | | | | G = 1.00 | G = 1.00 | | | |
| 3741845 | 11482 | 10853382 | T/C | T = 0.54 | T = 0.49 | 0.0877 | 0.81 | C |
| | | | | C = 0.46 | C = 0.51 | | | |
| 2110096 | 14544 | 10856444 | C/T | C = 1.00 | C = 1.00 | | | |
| | | | | T = 0.00 | T = 0.00 | | | |
| 759055 | 15688 | 10857588 | T/A | T = 0.45 | T = 0.37 | 0.0084 | 0.72 | A |
| | | | | A = 0.55 | A = 0.63 | | | |
| 589377 | 17311 | 10859211 | G/C | G = 0.01 | G = 0.00 | | | |
| | | | | C = 0.99 | C = 1.00 | | | |
| 7960194 | 17831 | 10859731 | G/T | G = 1.00 | G = 1.00 | | | |
| | | | | T = 0.00 | T = 0.00 | | | |
| 7978242 | 20012 | 10861912 | A/G | A = 0.62 | A = 0.68 | 0.0611 | 1.27 | A |
| | | | | G = 0.38 | G = 0.32 | | | |
| 601051 | 21997 | 10863897 | G/A | G = 0.01 | G = 0.00 | | | |
| | | | | A = 0.99 | A = 1.00 | | | |
| 4262797 | 22861 | 10864761 | A/G | A = 0.61 | A = 0.54 | 0.0391 | 0.76 | G |
| | | | | G = 0.39 | G = 0.46 | | | |
| 2215714 | 23470 | 10865370 | A/G | A = 0.00 | A = 0.00 | | | |
| | | | | G = 1.00 | G = 1.00 | | | |
| 1373434 | 23515 | 10865415 | G/A | G = 1.00 | G = 1.00 | | | |
| | | | | A = 0.00 | A = 0.00 | | | |
| 2215715 | 23863 | 10865763 | G/A | G = 1.00 | G = 1.00 | | | |
| | | | | A = 0.00 | A = 0.00 | | | |
| 612456 | 24108 | 10866008 | C/A | C = 0.03 | C = 0.02 | | | |
| | | | | A = 0.97 | A = 0.98 | | | |
| 612808 | 24138 | 10866038 | T/C | T = 0.20 | T = 0.11 | 0.0001 | 0.52 | C |
| | | | | C = 0.80 | C = 0.89 | | | |
| 689118 | 26469 | 10868369 | T/C | T = 0.68 | T = 0.71 | 0.3485 | 1.15 | T |
| | | | | C = 0.32 | C = 0.29 | | | |
| 597468 | 27769 | 10869669 | A/G | A = 0.99 | A = 1.00 | | | |
| | | | | G = 0.01 | G = 0.00 | | | |
| 592864 | 29683 | 10871583 | T/C | T = 0.10 | T = 0.11 | 0.9292 | 1.04 | T |
| | | | | C = 0.90 | C = 0.89 | | | |
| 640372 | 30491 | 10872391 | A/C | A = 0.96 | A = 0.96 | 0.6780 | 1.21 | A |
| | | | | C = 0.04 | C = 0.04 | | | |
| 7966559 | 30745 | 10872645 | A/T | A = 0.79 | A = | | | |
| | | | | T = 0.21 | T = | | | |
| 654834 | 31429 | 10873329 | T/C | T = 0.04 | T = 0.03 | 0.3922 | 0.74 | C |
| | | | | C = 0.96 | C = 0.97 | | | |
| 4763216 | 31779 | 10873679 | C/G | C = 0.50 | C = 0.45 | 0.0951 | 0.82 | G |
| | | | | G = 0.50 | G = 0.55 | | | |
| 668521 | 32194 | 10874094 | A/C | A = 0.98 | A = 0.99 | | | |
| | | | | C = 0.02 | C = 0.01 | | | |
| 669503 | 32441 | 10874341 | T/C | T = 0.96 | T = 0.96 | 0.4685 | 1.26 | T |
| | | | | C = 0.04 | C = 0.04 | | | |
| 3906864 | 32454 | 10874354 | T/C | T = 0.99 | T = 1.00 | | | |
| | | | | C = 0.01 | C = 0.00 | | | |
| 3906863 | 32459 | 10874359 | C/T | C = 0.34 | C = 0.41 | 0.0288 | 1.35 | C |
| | | | | T = 0.66 | T = 0.59 | | | |
| 7957888 | 35151 | 10877051 | A/T | A = 0.87 | A = 0.93 | 0.0155 | 2.12 | A |
| | | | | T = 0.13 | T = 0.07 | | | |
| 9300230 | 35362 | 10877262 | A/T | A = 1.00 | A = 1.00 | | | |
| | | | | T = 0.00 | T = 0.00 | | | |
| 7306214 | 35630 | 10877530 | G/C | G = 1.00 | G = 1.00 | | | |
| | | | | C = 0.00 | C = 0.00 | | | |
| 763839 | 36930 | 10878830 | G/C | G = 0.82 | G = 0.88 | 0.0047 | 1.61 | G |
| | | | | C = 0.18 | C = 0.12 | | | |
| 2418105 | 37490 | 10879390 | G/A | G = 0.21 | G = 0.16 | 0.0198 | 0.69 | A |
| | | | | A = 0.79 | A = 0.84 | | | |
| 666841 | 38432 | 10880332 | C/T | C = 0.89 | C = 0.93 | 0.0180 | 1.72 | C |
| | | | | T = 0.11 | T = 0.07 | | | |
| 3851578 | 38688 | 10880588 | A/G | A = 0.89 | A = 0.93 | 0.0243 | 1.67 | A |
| | | | | G = 0.11 | G = 0.07 | | | |
| 7138797 | 39524 | 10881424 | T/C | T = 0.97 | T = 0.97 | 0.9815 | 0.99 | T |
| | | | | C = 0.03 | C = 0.03 | | | |
| 7295252 | 41580 | 10883480 | A/C | A = 1.00 | A = 1.00 | | | |
| | | | | C = 0.00 | C = 0.00 | | | |
| 2418106 | 42531 | 10884431 | T/C | T = 0.35 | T = 0.30 | 0.1784 | 0.80 | C |
| | | | | C = 0.65 | C = 0.70 | | | |
| 7299578 | 42665 | 10884565 | A/G | A = 0.30 | A = 0.23 | 0.0036 | 0.67 | G |
| | | | | G = 0.70 | G = 0.77 | | | |
| 621112 | 43038 | 10884938 | A/G | A = 0.87 | A = 0.92 | 0.0086 | 1.69 | A |
| | | | | G = 0.13 | G = 0.08 | | | |
| 3863320 | 44183 | 10886083 | G/A | G = 0.52 | G = 0.49 | 0.2271 | 0.87 | A |
| | | | | A = 0.48 | A = 0.51 | | | |
| 1373432 | 46271 | 10888171 | A/T | A = 0.00 | A = 0.00 | | | |
| | | | | T = 1.00 | T = 1.00 | | | |
| 1047699 | 49075 | 10890975 | C/T | C = 0.79 | C = 0.85 | 0.0062 | 1.53 | C |
| | | | | T = 0.21 | T = 0.15 | | | |
| 1063193 | 49147 | 10891047 | C/T | C = 0.75 | C = | | | |
| | | | | T = 0.25 | T = | | | |
| 2232959 | 49180 | 10891080 | C/G | C = 1.00 | C = 1.00 | | | |
| | | | | G = 0.00 | G = 0.00 | | | |
| 2227296 | 50301 | 10892201 | A/G | A = 0.77 | A = 0.77 | 0.9934 | 1.00 | A |
| | | | | G = 0.23 | G = 0.23 | | | |
| 1548804 | 50773 | 10892673 | G/A | G = 0.24 | G = 0.15 | 0.0005 | 0.54 | A |
| | | | | A = 0.76 | A = 0.85 | | | |
| 2232958 | 51243 | 10893143 | G/A | G = 1.00 | G = 1.00 | | | |
| | | | | A = 0.00 | A = 0.00 | | | |
| 2232957 | 51530 | 10893430 | T/C | T = 1.00 | T = 1.00 | | | |
| | | | | C = 0.00 | C = 0.00 | | | |
| 2232956 | 52107 | 10894007 | A/G | A = 0.87 | A = 0.92 | 0.0170 | 1.64 | A |
| | | | | G = 0.13 | G = 0.08 | | | |
| 1972571 | 52821 | 10894721 | A/G | A = 0.13 | A = 0.13 | 0.8117 | 1.04 | A |
| | | | | G = 0.87 | G = 0.87 | | | |
| 3759250 | 53341 | 10895241 | T/C | T = 0.99 | T = 1.00 | | | |
| | | | | C = 0.01 | C = 0.00 | | | |
| 3759249 | 53376 | 10895276 | C/G | C = 1.00 | C = 1.00 | | | |
| | | | | G = 0.00 | G = 0.00 | | | |
| 1541525 | 54047 | 10895947 | C/T | C = 0.00 | C = 0.00 | | | |
| | | | | T = 1.00 | T = 1.00 | | | |
| 2098248 | 54392 | 10896292 | T/C | T = 0.00 | T = 0.00 | | | |
| | | | | C = 1.00 | C = 1.00 | | | |
| 2900550 | 54482 | 10896382 | T/A | T = 0.89 | T = 0.93 | 0.0535 | 1.59 | T |
| | | | | A = 0.11 | A = 0.07 | | | |
| 7302130 | 55468 | 10897368 | C/A | C = 0.36 | C = 0.36 | 0.9550 | 1.01 | C |
| | | | | A = 0.64 | A = 0.64 | | | |
| 4763583 | 56990 | 10898890 | A/G | A = | A = | | | |
| | | | | G = | G = | | | |
| 4360778 | 57712 | 10899612 | A/T | A = 0.00 | A = 0.01 | | | |
| | | | | T = 1.00 | T = 0.99 | | | |
| 1607695 | 59667 | 10901567 | C/T | C = 0.00 | C = 0.01 | | | |
| | | | | T = 1.00 | T = 0.99 | | | |
| 1607694 | 59684 | 10901584 | T/C | T = 0.81 | T = 0.83 | 0.3469 | 1.17 | T |
| | | | | C = 0.19 | C = 0.17 | | | |
| 2192139 | 62043 | 10903943 | A/C | A = 0.30 | A = 0.28 | 0.4555 | 0.89 | C |
| | | | | C = 0.70 | C = 0.72 | | | |
| 7978300 | 63293 | 10905193 | T/C | T = 0.36 | T = | | | |
| | | | | C = 0.64 | C = | | | |
| 7397871 | 63485 | 10905385 | G/T | G = 0.00 | G = 0.00 | | | |
| | | | | T = 1.00 | T = 1.00 | | | |
| 4763217 | 63778 | 10905678 | C/T | C = 0.89 | C = 0.90 | 0.8698 | 1.05 | C |
| | | | | T = 0.11 | T = 0.10 | | | |
| 2159900 | 64222 | 10906122 | A/G | A = 0.02 | A = 0.02 | | | |
| | | | | G = 0.98 | G = 0.98 | | | |
| 10772370 | 65722 | 10907622 | A/G | A = 0.31 | A = 0.27 | 0.1478 | 0.82 | G |
| | | | | G = 0.69 | G = 0.73 | | | |
| 7398682 | 66315 | 10908215 | C/T | C = 1.00 | C = 1.00 | | | |
| | | | | T = 0.00 | T = 0.00 | | | |
| 2900551 | 66829 | 10908729 | A/G | A = 0.00 | A = 0.01 | | | |
| | | | | G = 1.00 | G = 0.99 | | | |
| 2900552 | 66966 | 10908866 | C/T | C = 0.61 | C = 0.59 | 0.5150 | 0.92 | T |
| | | | | T = 0.39 | T = 0.41 | | | |
| 2418214 | 66971 | 10908871 | A/C | A = 0.51 | A = 0.57 | 0.0565 | 1.27 | A |
| | | | | C = 0.49 | C = 0.43 | | | |
| 2418215 | 67013 | 10908913 | T/C | T = 0.82 | T = 0.86 | 0.1232 | 1.29 | T |
| | | | | C = 0.18 | C = 0.14 | | | |
| 965243 | 70375 | 10912275 | A/T | A = 0.82 | A = 0.85 | 0.1637 | 1.25 | A |
| | | | | T = 0.18 | T = 0.15 | | | |
| 1117548 | 74118 | 10916018 | T/A | T = 1.00 | T = 0.99 | | | |
| | | | | A = 0.00 | A = 0.01 | | | |
| 1520225 | 75224 | 10917124 | C/T | C = 0.27 | C = 0.22 | 0.0956 | 0.79 | T |
| | | | | T = 0.73 | T = 0.78 | | | |
| 1520226 | 75236 | 10917136 | T/C | T = 0.99 | T = 0.98 | | | |
| | | | | C = 0.01 | C = 0.02 | | | |
| 1520227 | 75246 | 10917146 | G/C | G = 0.91 | G = 0.82 | 0.00001 | 0.46 | C |
| | | | | C = 0.09 | C = 0.18 | | | |
| 971919 | 75812 | 10917712 | C/A | C = 0.00 | C = 0.00 | | | |
| | | | | A = 1.00 | A = 1.00 | | | |
| 2159901 | 78968 | 10920868 | C/T | C = 0.99 | C = 0.99 | | | |
| | | | | T = 0.01 | T = 0.01 | | | |
| 2159902 | 78998 | 10920898 | T/C | T = 0.54 | T = 0.51 | 0.3599 | 0.90 | C |
| | | | | C = 0.46 | C = 0.49 | | | |
| 2110099 | 79328 | 10921228 | A/C | A = 0.60 | A = 0.63 | 0.4080 | 1.11 | A |
| | | | | C = 0.40 | C = 0.37 | | | |
| 7314847 | 80922 | 10922822 | T/C | T = 0.51 | T = 0.49 | 0.6059 | 0.94 | C |
| | | | | C = 0.49 | C = 0.51 | | | |
| 7296003 | 81055 | 10922955 | T/C | T = 0.63 | T = 0.65 | 0.4799 | 1.09 | T |
| | | | | C = 0.37 | C = 0.35 | | | |
| 4281556 | 81412 | 10923312 | G/A | G = 0.85 | G = 0.87 | 0.5327 | 1.17 | G |
| | | | | A = 0.15 | A = 0.13 | | | |
| 4763219 | 81785 | 10923685 | A/G | A = | A = | | | |
| | | | | G = | G = | | | |
| 3851579 | 82079 | 10923979 | G/A | G = | G = 0.34 | | | |
| | | | | A = | A = 0.66 | | | |
| 3851580 | 82087 | 10923987 | G/A | G = 0.56 | G = 0.54 | 0.5618 | 0.93 | A |
| | | | | A = 0.44 | A = 0.46 | | | |
| 1049119 | 82958 | 10924858 | A/G | A = 0.00 | A = 0.00 | | | |
| | | | | G = 1.00 | G = 1.00 | | | |
| 2298866 | 83351 | 10925251 | T/G | T = 0.72 | T = 0.69 | 0.3240 | 0.87 | G |
| | | | | G = 0.28 | G = 0.31 | | | |
| 2298865 | 83442 | 10925342 | C/T | C = 0.95 | C = 0.97 | 0.1804 | 1.67 | C |
| | | | | T = 0.05 | T = 0.03 | | | |
| 2298864 | 83472 | 10925372 | A/C | A = 0.36 | A = 0.34 | 0.5936 | 0.93 | C |
| | | | | C = 0.64 | C = 0.66 | | | |
| 2298863 | 83966 | 10925866 | A/G | A = 1.00 | A = 1.00 | | | |
| | | | | G = 0.00 | G = 0.00 | | | |
| 3180393 | 84414 | 10926314 | T/C | T = 1.00 | T = 1.00 | | | |
| | | | | C = 0.00 | C = 0.00 | | | |
| 2070837 | 86563 | 10928463 | C/G | C = 0.43 | C = 0.43 | 0.9403 | 0.99 | C |
| | | | | G = 0.57 | G = 0.57 | | | |
| 7956204 | 86788 | 10928688 | G/A | G = 0.86 | G = | | | |
| | | | | A = 0.14 | A = | | | |
| 2418216 | 86796 | 10928696 | T/G | T = 1.00 | T = 1.00 | | | |
| | | | | G = 0.00 | G = 0.00 | | | |
| 3741844 | 87634 | 10929534 | G/A | G = 0.50 | G = 0.48 | 0.3827 | 0.90 | A |
| | | | | A = 0.50 | A = 0.52 | | | |
| 4262798 | 88530 | 10930430 | G/A | G = 0.60 | G = 0.62 | 0.4130 | 1.10 | G |
| | | | | A = 0.40 | A = 0.38 | | | |
| 2418217 | 89202 | 10931102 | A/G | A = 0.53 | A = 0.52 | 0.6562 | 0.95 | G |
| | | | | G = 0.47 | G = 0.48 | | | |
| 2418218 | 89632 | 10931532 | T/C | T = | T = | | | |
| | | | | C = | C = | | | |
| 7137492 | 89697 | 10931597 | T/C | T = 0.75 | T = 0.76 | 0.7043 | 1.07 | T |
| | | | | C = 0.25 | C = 0.24 | | | |
| 2110100 | 89723 | 10931623 | G/A | G = 0.53 | G = 0.52 | 0.5403 | 0.93 | A |
| | | | | A = 0.47 | A = 0.48 | | | |
| 1013312 | 91063 | 10932963 | C/G | C = 0.29 | C = 0.26 | 0.1928 | 0.84 | G |
| | | | | G = 0.71 | G = 0.74 | | | |
| 4579993 | 91335 | 10933235 | C/G | C = 0.00 | C = 0.00 | | | |
| | | | | G = 1.00 | G = 1.00 | | | |
| 1013313 | 91504 | 10933404 | T/C | T = 0.61 | T = 0.58 | 0.3893 | 0.90 | C |
| | | | | C = 0.39 | C = 0.42 | | | |
| 7397106 | 91619 | 10933519 | C/T | C = 0.02 | C = 0.04 | | | |
| | | | | T = 0.98 | T = 0.96 | | | |
| 2215716 | 93715 | 10935615 | A/G | A = 0.45 | A = 0.36 | 0.0372 | 0.69 | G |
| | | | | G = 0.55 | G = 0.64 | | | |
| 2192140 | 93945 | 10935845 | T/C | T = 0.00 | T = 0.00 | | | |
| | | | | C = 1.00 | C = 1.00 | | | |
| 4763589 | 94235 | 10936135 | T/C | T = 0.53 | T = 0.52 | 0.6806 | 0.95 | C |
| | | | | C = 0.47 | C = 0.48 | | | |
| 1468697 | 95851 | 10937751 | T/C | T = 0.02 | T = 0.01 | | | |
| | | | | C = 0.98 | C = 0.99 | | | |
| 2070837 | 130860 | 10972760 | C/G | C = 0.43 | C = 0.43 | 0.9403 | 0.99 | C |
| | | | | G = 0.57 | G = 0.57 | | | |
| 3180393 | 132815 | 10974715 | T/C | T = 1.00 | T = 1.00 | | | |
| | | | | C = 0.00 | C = 0.00 | | | |
| 2298865 | 133778 | 10975678 | C/T | C = 0.95 | C = 0.97 | 0.1804 | 1.67 | C |
| | | | | T = 0.05 | T = 0.03 | | | |

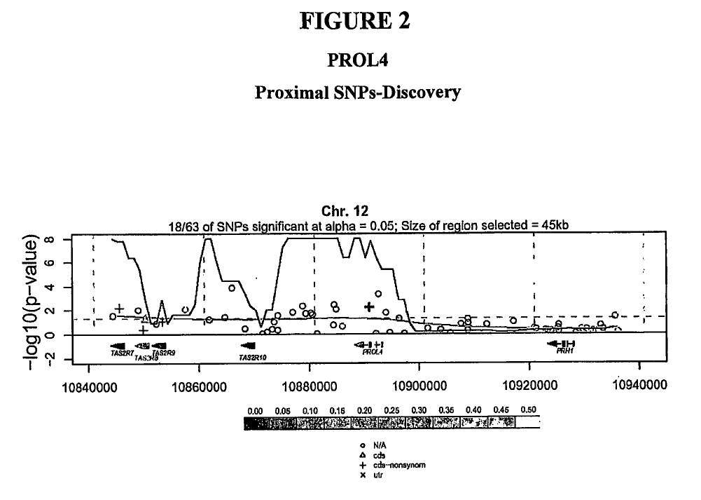

Allelotyping results were considered particularly significant with a calculated p-value of less than or equal to 0.05 for allelotype results. These values are indicated in bold. The allelotyping p-values were plotted in FIG. 2. The position of each SNP on the chromosome is presented on the x-axis. The y-axis gives the negative logarithm (base 10) of the p-value comparing the estimated allele in the case group to that of the control group. The minor allele frequency of the control group for each SNP designated by an X or other symbol on the graphs in FIG. 2 can be determined by consulting Table 15. For example, the left-most X on the left graph is at position 10842129. By proceeding down the Table from top to bottom and across the graphs from left to right the allele frequency associated with each symbol shown can be determined.

To aid the interpretation, multiple lines have been added to the graph. The broken horizontal lines are drawn at two common significance levels, 0.05 and 0.01. The vertical broken lines are drawn every 20 kb to assist in the interpretation of distances between SNPs. Two other lines are drawn to expose linear trends in the association of SNPs to the disease. The light gray line (or generally bottom-most curve) is a nonlinear smoother through the data points on the graph using a local polynomial regression method (W. S. Cleveland, E. Grosse and W. M. Shyu (1992) Local regression models. Chapter 8 of Statistical Models in S eds J. M. Chambers and T. J. Hastie, Wadsworth & Brooks/Cole.). The black line provides a local test for excess statistical significance to identify regions of association. This was created by use of a 10 kb sliding window with 1 kb step sizes. Within each window, a chi-square goodness of fit test was applied to compare the proportion of SNPs that were significant at a test wise level of 0.01, to the proportion that would be expected by chance alone (0.05 for the methods used here). Resulting p-values that were less than 10⁻⁸ were truncated at that value.

Finally, the exons and introns of the genes in the covered region are plotted below each graph at the appropriate chromosomal positions. The gene boundary is indicated by the broken horizontal line. The exon positions are shown as thick, unbroken bars. An arrow is place at the 3′ end of each gene to show the direction of transcription.

### Example 5

### GRID2 Proximal SNPs

It has been discovered that a polymorphic variation (rs1948017) in a gene encoding GRID2 is associated with the occurrence of low BMD (see Examples 1 and 2). One hundred five additional allelic variants proximal to rs1948017 were identified and subsequently allelotyped in low BMD case and high BMD control sample sets as described in Examples 1 and 2. The polymorphic variants are set forth in Table 16. The chromosome position provided in column four of Table 16 is based on Genome “Build 34” of NCBI's GenBank.

TABLE 16

| | Position in | | | | | |
|---|---|---|---|---|---|---|
| | SEQ ID | | Chromosome | | | |
| dbSNP | NO: 3 | Chromosome | Position | Alleles (A1/A2) | genome_letter | deduced_iupac |
| 1433661 | 206 | 4 | 94744306 | t/c | g | R |
| 1485009 | 243 | 4 | 94744343 | a/g | g | R |
| 7681947 | 2467 | 4 | 94746567 | t/c | t | Y |
| 1816432 | 3550 | 4 | 94747650 | a/g | t | Y |
| 1485018 | 4994 | 4 | 94749094 | c/g | g | S |
| 1485017 | 5167 | 4 | 94749267 | g/t | c | M |
| 7438397 | 5193 | 4 | 94749293 | c/t | c | Y |
| 6834311 | 5273 | 4 | 94749373 | g/a | g | R |
| 1368717 | 5733 | 4 | 94749833 | a/g | c | Y |
| 1017391 | 7817 | 4 | 94751917 | a/c | g | K |
| 2870701 | 7818 | 4 | 94751918 | t/a | a | W |
| 7679839 | 8612 | 4 | 94752712 | t/g | t | K |
| 1385404 | 9158 | 4 | 94753258 | a/g | a | R |
| 1368716 | 9285 | 4 | 94753385 | g/a | c | Y |
| 4693316 | 10680 | 4 | 94754780 | g/a | a | R |
| 1905707 | 11866 | 4 | 94755966 | t/c | c | Y |
| 1905708 | 11958 | 4 | 94756058 | a/g | a | R |
| 1905709 | 12044 | 4 | 94756144 | a/t | t | W |
| 3912442 | 12753 | 4 | 94756853 | g/t | t | K |
| 2082553 | 15585 | 4 | 94759685 | a/g | c | Y |
| 6831638 | 17299 | 4 | 94761399 | c/t | c | Y |
| 5860329 | 18816 | 4 | 94762916 | —/g | g | N |
| 2870702 | 24022 | 4 | 94768122 | c/t | t | Y |
| 2870703 | 24994 | 4 | 94769094 | c/t | t | Y |
| 1948016 | 26637 | 4 | 94770737 | g/t | t | K |
| 6835836 | 27635 | 4 | 94771735 | c/g | g | S |
| 1994253 | 28773 | 4 | 94772873 | t/c | t | Y |
| 1905710 | 29430 | 4 | 94773530 | a/t | a | W |
| 1485019 | 29876 | 4 | 94773976 | t/a | t | W |
| 978191 | 30364 | 4 | 94774464 | c/t | a | R |
| 1385405 | 31057 | 4 | 94775157 | c/a | t | K |
| 7694361 | 31782 | 4 | 94775882 | c/t | t | Y |
| 1905711 | 33400 | 4 | 94777500 | a/c | c | M |
| 1905734 | 35588 | 4 | 94779688 | a/c | t | K |
| 1485012 | 37663 | 4 | 94781763 | c/g | c | S |
| 1485013 | 37865 | 4 | 94781965 | t/c | c | Y |
| 4692981 | 38218 | 4 | 94782318 | a/t | a | W |
| 7670552 | 39375 | 4 | 94783475 | t/c | c | Y |
| 7670932 | 39559 | 4 | 94783659 | c/t | c | Y |
| 7688091 | 39833 | 4 | 94783933 | a/g | g | R |
| 7440540 | 40135 | 4 | 94784235 | a/g | g | R |
| 2171000 | 41698 | 4 | 94785798 | g/a | t | Y |
| 2870704 | 42249 | 4 | 94786349 | t/c | t | Y |
| 7655758 | 42571 | 4 | 94786671 | g/c | c | S |
| 7661436 | 42977 | 4 | 94787077 | g/a | g | R |
| 7662289 | 43548 | 4 | 94787648 | t/c | c | Y |
| 7667044 | 43631 | 4 | 94787731 | g/a | a | R |
| 7691929 | 43705 | 4 | 94787805 | t/c | t | Y |
| 5860330 | 43817 | 4 | 94787917 | tt/t— | t— | N |
| 901013 | 44374 | 4 | 94788474 | a/c | g | K |
| 901012 | 44464 | 4 | 94788564 | a/c | g | K |
| 901011 | 44788 | 4 | 94788888 | t/c | g | R |
| 1948018 | 48962 | 4 | 94793062 | a/g | c | Y |
| 2870705 | 48993 | 4 | 94793093 | t/g | t | K |
| 1948017 | 49110 | 4 | 94793210 | c/t | a | R |
| 1905733 | 49434 | 4 | 94793534 | c/t | a | R |
| 1385408 | 49523 | 4 | 94793623 | t/g | t | K |
| 1385409 | 49742 | 4 | 94793842 | c/g | c | S |
| 1385410 | 49907 | 4 | 94794007 | g/c | g | S |
| 1485026 | 50028 | 4 | 94794128 | g/c | g | S |
| 1485027 | 50089 | 4 | 94794189 | g/t | t | K |
| 2904483 | 51588 | 4 | 94795688 | c/a | a | M |
| 1385406 | 52899 | 4 | 94796999 | c/a | a | M |
| 1905732 | 54088 | 4 | 94798188 | a/c | t | K |
| 2046418 | 56538 | 4 | 94800638 | c/t | a | R |
| 2200377 | 59071 | 4 | 94803171 | t/c | g | R |
| 1905731 | 59110 | 4 | 94803210 | a/c | t | K |
| 1905730 | 59178 | 4 | 94803278 | a/g | c | Y |
| 975713 | 61087 | 4 | 94805187 | t/c | a | R |
| 6820985 | 61300 | 4 | 94805400 | c/g | c | S |
| 7670441 | 62171 | 4 | 94806271 | t/c | c | Y |
| 6810794 | 62783 | 4 | 94806883 | t/a | t | W |
| 7676623 | 62983 | 4 | 94807083 | t/c | c | Y |
| 1154861 | 63908 | 4 | 94808008 | c/t | t | Y |
| 1032125 | 64088 | 4 | 94808188 | g/t | g | K |
| 1485022 | 64941 | 4 | 94809041 | a/t | a | W |
| 1485024 | 65050 | 4 | 94809150 | a/g | g | R |
| 3913651 | 68953 | 4 | 94813053 | c/t | c | Y |
| 4693319 | 70093 | 4 | 94814193 | t/c | c | Y |
| 1872383 | 71308 | 4 | 94815408 | c/a | g | K |
| 2200376 | 73009 | 4 | 94817109 | t/a | t | W |
| 7668090 | 74002 | 4 | 94818102 | a/g | g | R |
| 7692930 | 74294 | 4 | 94818394 | t/c | t | Y |
| 967096 | 74879 | 4 | 94818979 | g/c | g | S |
| 6822249 | 76936 | 4 | 94821036 | g/t | t | K |
| 6532405 | 77195 | 4 | 94821295 | g/a | g | R |
| 1017897 | 77683 | 4 | 94821783 | t/c | c | Y |
| 7672674 | 78283 | 4 | 94822383 | t/c | t | Y |
| 7694568 | 78331 | 4 | 94822431 | c/t | c | Y |
| 2904484 | 79362 | 4 | 94823462 | g/c | c | S |
| 7340830 | 80357 | 4 | 94824457 | c/t | c | Y |
| 1485033 | 80653 | 4 | 94824753 | t/c | g | R |
| 2870706 | 80840 | 4 | 94824940 | a/g | g | R |
| 1905729 | 83203 | 4 | 94827303 | a/g | c | Y |
| 4693320 | 85405 | 4 | 94829505 | t/c | t | Y |
| 6848749 | 86441 | 4 | 94830541 | g/t | t | K |
| 6532406 | 86967 | 4 | 94831067 | g/a | g | R |
| 6532407 | 87121 | 4 | 94831221 | t/c | t | Y |
| 1905728 | 89617 | 4 | 94833717 | t/c | g | R |
| 6819866 | 90969 | 4 | 94835069 | t/a | a | W |
| 1905727 | 94249 | 4 | 94838349 | g/c | g | S |
| 7674069 | 95811 | 4 | 94839911 | t/g | t | K |
| 1905724 | 96690 | 4 | 94840790 | t/g | c | M |
| 1905723 | 96731 | 4 | 94840831 | a/g | c | Y |
| 1485020 | 97267 | 4 | 94841367 | c/g | c | S |
| 6814101 | 97414 | 4 | 94841514 | t/g | g | K |

Assay for Verifying and Allelotyping SNPs

the methods used to verify and allelotype the proximal SNPs of Table 16 are the same methods described in Examples 1 and 2 herein. The primers and probes used in these assays are provided in Table 17 and Table 18, respectively.

TABLE 17

| | | |
|---|---|---|
| DbSNP | | |
| rs# | Forward PCR primer | Reverse PCR primer |

| | | |
|---|---|---|
| 1433661 | CTCCCAAAGCCATGGGATTC | CAGGAATGCAATCTAAGGCC |
| 1485009 | CCTAACCCTCTTCTGGAAAA | GACCTATAAAGACAGGAATGC |
| 7681947 | ACCAGAACAGGACGAATTAG | CCCCAATGTTATGCATACTC |
| 1816432 | GTTCTAGCCTTACTGAGATAG | GCTTAAAGACCTTCAAACTAC |
| 1485018 | AAAGATCGCTTGAAACTGGG | TTGCTCTGTTGTCAAGGCTG |
| 1485017 | TGACCAGGCTGGTCTAAAAC | TAATCCCAGCAGTTTGGGAG |
| 7438397 | GAGATCTAGCAATCCTCCTG | GAAACCTAATTTTGAGCCTG |
| 6834311 | TCATTGTGTTACAGATGCTC | CCAGGCTCAAAATTAGGTTTC |
| 1368717 | AAATCTGCAGACAAGGACAG | AGCAACCTGGTTTTAAGAGC |
| 1017391 | ATGCACAAATGACAGTAGGG | GGACATTGTAGTAGTCAGGG |
| 2870701 | ATGCACAAATGACAGTAGGG | GGACATTGTAGTAGTCAGGG |
| 7679839 | CCACAGGAACTATTTGCTGG | GGAACAGCACAAGCAAAAAC |
| 1385404 | CCCAGCATCCTTGCTAATAC | CCTGTCAAGATGCATAGGAG |
| 1368716 | TGGGAGAACTGGGAAAATGG | TGCTACATACTGTAGCAGCC |
| 4693316 | ATCCTGTACTGCACTTCAGC | ACCCTCCATGGGAGTTTTTC |
| 1905707 | TTTTCTGTAGTTTCCCGAGC | AATGCAATCCACAGAGCAAG |
| 1905708 | GGAACAAATGAAGATAGATG | GGACCTATGTTTTATGAGTTC |
| 1905709 | TAGAATAGTTTCTCAGTGTC | GAACTCATAAAACATAGGTCC |
| 3912442 | CATAACTCAGGCAAAACAAC | GTTGAAAAAGCTCTATATTGG |
| 2082553 | TGGAGCCCACTCAGATATTC | CCCTGGAACATGTAAATGTG |
| 6831638 | CCATATACTTACCTTTACTG | AATGTGAGTTCTGAAGATGG |
| 5860329 | CCATGCTGGCTTTTGTGAAC | TCCTGACCCTTAGTCCAATG |
| 2870702 | CAAAGAATGAGAGGGAAGGC | TGTGGCCTACCAGAATTTGC |
| 2870703 | AATAAAGAGGACAGCAGGAG | CACAGTTGTACTCCTAGCTC |
| 1948016 | GAATGAATGCAGTGTGCCAG | TGAACCTATCTTGCAGGTGG |
| 6835836 | TTATTTAGGAAGCACTCAGC | GAGTCCAAGACTAGTAAAGAG |
| 1994253 | TTCACCTTTCTGTGTTCTCC | AAGAAGAAGAACAACTGTAC |
| 1905710 | CATTAAGTGAAGAGCTATTCC | CTTAAAAGCTATTCGTTAAGC |
| 1485019 | TTTAAGCTCCCCAAAAGGTG | TACTGTGACATTCCCTTCTC |
| 978191 | AGGTCTTATCTCTTGGAATG | CCCATTCAGATATATAGACAG |
| 1385405 | CATTCAGACCTGTGACAGGA | TGATGGGACTGTTCTTCAGG |
| 7694361 | CTAGTGATTCTGTGAATGTTG | CAATGTGATTGTGTTGGAAG |
| 1905711 | CAGTTAACACTCCATATCCAC | GAGCCTCAGTTTGTTGAATC |
| 1905734 | AATTTCAAGATCAGAGATCC | CCTAACATGAGTCCATTCTG |
| 1485012 | ATACACCTATGCTAAGAAGC | GCAGAAAAACTAGTACGATAC |
| 1485013 | ACACATCATAGTTTTACTGC | GGATGAAACTATGTGAAAGC |
| 4692981 | GCTAGCCTGGAATCAAACAC | CCGAAAACAAAAGCACCCTG |
| 7670552 | TTTGGCTGCAATCAACCATG | ATCCCAAGAGAAATGAAAAC |
| 7670932 | CCTTGAAAGCATGCTAAGGG | TGAGTCCCTATAGATTTGTC |
| 7688091 | TGGATTGTGCACTTTGGATG | ACCAGACTCTCTCTTTTTTA |
| 7440540 | TGCACAGACTGTTAAGGGAG | TCATCCTTTAAGCCTTGCCG |
| 2171000 | TGAGCCAAGTATGGAAAAAC | CTGGGATAATTGTTGGGATC |
| 2870704 | ATGCTCTTGGAGCTCAGAAG | ACATATTTCCCTGACCTCCC |
| 7655758 | GTCAAAACCTCTTACTGTGC | CATGGCAGCCTTACCAAAGA |
| 7661436 | ACTGAAATTCCTTGCTGAAC | TCTCCAGGTAAACTTCCAAG |
| 7662289 | AAAACAGTGAAAGACCTTGC | TATTGAGGCCGAGTAGTCTG |
| 7667044 | ACTGATAAAACAAAGGAGGG | GAAAGCAAGGTCTTTCACTG |
| 7691929 | TTTCTCAGCATATGTTCCAG | ATCAGTCTCAGGGAAGATGC |
| 5860330 | CTGCCTAGATGAAACAAATG | CTGCTGGAACATATGCTGAG |
| 901013 | AAGGTGCATCCCTCAGAAAG | GCCTGGCTGATCTAAGGTTT |
| 901012 | CTGAAATAGTGAGGGTAGTG | AAGATGTGGAGAGAACAAGG |
| 901011 | AAAAGGGAAGGGAAGTGTGG | ACTCTCCTCCAAGAGCTTTC |
| 1948018 | TTTATCAGCAAGCATGTGAG | GTTTTCTGGAAAACTTTTACC |
| 2870705 | TAGTGCATGAGAAAGTCTAC | TTGCTTATCTGACTGGCAAG |
| 1948017 | GTTTAACAGCAACCATTGAGG | CCCCAAAGGTATGTTAAGAG |
| 1905733 | TGGAAGAGTATTAACTTCTG | TTAGTCTATTGATTGAAAGC |
| 1385408 | ACTCTTCCAAAGACTGATGC | GGATGTGGGGTTATAAGAAC |
| 1385409 | TTGCCTCTTCCAGTCTATTG | CAGAGGAGAGACATGATCAG |
| 1385410 | AGAGACAGAAGAGACACTAG | CCCCATCCAAATACATTGCC |
| 1485026 | GAGAAGAAGGCATATGTGAG | CTCACTCTTTCTTCTGTCTG |
| 1485027 | TGGTGATGATAGGTGCTGTG | ACACAGGTGCTTTCTAATTG |
| 2904483 | CTTCTCTCCATGAAACTCAG | AGGAGATATCAAGACACCAG |
| 1385406 | CTCCATTATCTGTTTGCAGT | CTCTGAGTAGTGTTTTCTGG |
| 1905732 | CTGTTCCTTGTTTCTTCTGC | GTGGTGTTTGCCATTAAAAG |
| 2046418 | AGGAGAGAAGTCTATGTCCC | TTCTTACATTCTAGAGCCTT |
| 2200377 | GGATGGACTATTGTTTTCTT | CCTATCTTGATTAGATTACAG |
| 1905731 | TCACTGCTTAGAAACTAATC | AAACACCTTTCTGAGAATTG |
| 1905730 | AAACTGAAGCAGATTCTGAC | CTTTGTTCTTTCAATTCTCAG |
| 975713 | GCTAATTTTCCTAGGATCTC | AGTGAGCCCCAAAATCTGTG |
| 6820985 | CTAAGCCTAATAAAGGAGGG | ATATATAGAAAAGACTGGAG |
| 7670441 | CAATAATGACATGCTCTTAC | CGTATAGCAATCATACACAAG |
| 6810794 | ATGATGGAGCTCCAGAAAAG | CAGAGATATTTAGTGGCAAC |
| 7676623 | TTCAGAGCTCACTTTCCAAC | GGGGCTACCAAACTTAAAAG |
| 1154861 | GACACTGAATCCATAGATCG | CCTTTTTAAATAACCAGTGAC |
| 1032125 | CCTCATGCTTTAATAGGTAG | CACACATTTCTATAGTACTTG |
| 1485022 | AAATTAGGCTGAAGGAACAG | GCTTGATGGTTATTACAATGG |
| 1485024 | CGTTTCCACCACCTGGAAAT | CTGGCTTTCTTCCATTGTTG |
| 3913651 | CCTTGAGTATCTAAGAAAGGC | AAAATATACTTGTTTTGAGC |
| 4693319 | TCAAGAGTGGAAAGGGAGAG | CAGCTCCACTCACTATCTTG |
| 1872383 | TGGCCTCAAATGCATGTGTC | CTTTGGAGCTATCCAAATGG |
| 2200376 | GTGTTTGAGAAAGAAGTGTG | GAGTTCAGACAGAGTATGAG |
| 7668090 | GTAGGCCTGAGAATGCATTG | GACATTCCATTATTCCTCCC |
| 7692930 | ATGAGAACACATGGACACGG | TCTCCTAATGCTATCCCTCC |
| 967096 | GATTGGAAAAGGGCAACAGG | TCCATGAGAATGTTCCTCAG |
| 6822249 | AAAGGATGTTTCCATTTCTC | CCTAGAAAGGTAGTTGATGC |
| 6532405 | CTCAGGATCATTGAGACTTAG | CTTGAAAGATAACTGCGGAC |
| 1017897 | TGCCCAAACTGCAAATACAC | GCTACATTAAGTGAATTCTAC |
| 7672674 | CTACAACAGACAAGGATGGG | CAATGCCTTCAGCATTTTCC |
| 7694568 | CCATTTCTAATGGGTACCTC | TGCCATACACTACAACAGAC |
| 2904484 | ATTTTCACATTGCTTTGCCC | GAACAAGCAGAGCAAGTAAG |
| 7340830 | CAATCTCAAAACAGTGTTTAC | ACTAAGAAAGGAGAGAACAG |
| 1485033 | CTGGCTGGAATGTTAATAGG | TGTGCTCCTCAGTACATAGC |
| 2870706 | CCTGCAGGAAGAAAATAGGC | AGGGAAGCAAAACTAAGATG |
| 1905729 | GGGAATTACTCTAGCTCTTC | AGGAAAGAGTAGGCCAAATG |
| 4693320 | TCAAACTAGTAGCCCACAAA | GTATAGATTACAGTAGATGTG |
| 6848749 | CCATCTTTTTGTCTGCATTC | CAGTCAATTTTCATATTGCC |
| 6532406 | GCATGGCTCTTAGGAGAAAG | GCTGGAAGGGAAAATGGTAC |
| 6532407 | TTGTTTTCCTAGGCTCCAGC | TGGCTACAATAGGGAGACTG |
| 1905728 | GCAAGTTCATTTTCATATAGC | TAAAACAGTGTGAAATTTTG |
| | G | |
| 6819866 | CACACATAGCTCTTCTGTAAG | GCCTCGAGGAAAAAAAAATAG |
| 1905727 | AAGGGATAACAAGACAAATG | ACCCCATGATCTACTATTTC |
| 7674069 | CAGTTACTCCAAACTTACGG | GGTAACAAAGGCACTCAAGG |
| 1905724 | GCTCTCAGTGTCTTTTAATG | CGCATATGATTAGCTACTTC |
| 1905723 | GGGTTTATAAGCCTTTTCTTC | CTTGCTAACAATGAAAAGGTG |
| 1485020 | GTTGTCATTTTATGTCCTCCG | TAGAGTCACTGCCAATAAAC |
| 8814101 | GAGGACTTCAATGACTTTGC | TCAGACACAAACATCTGAAG |

TABLE 18

| | | |
|---|---|---|
| dbSNP | Extend | Term |
| rs# | Primer | Mix |

| | | |
|---|---|---|
| 1433661 | CATGGGATTCCAGGTGT | ACT |
| 1485009 | CCTCTTCTGGAAAATCTTAA | ACT |
| 7681947 | ACGAATTAGTCCAAGGA | ACT |
| 1816432 | TGAGATAGTTAATTTTGTTTTCCA | ACT |
| 1485018 | CAGTGAGCCATGACTGT | ACT |
| 1485017 | GGTCTAAAACTCCTGAGAT | CGT |
| 7438397 | CTCCTGCCTTGGCCTCC | ACG |
| 6834311 | GAAGAGAAAGAGATGGC | ACG |
| 1368717 | AGGGATCGGAAATTTAAGAAGAA | ACT |
| 1017391 | AATGACAGTAGGGATTATAGTAA | ACT |
| 2870701 | GACAGTAGGGATTATAGTAAG | CGT |
| 7679839 | ATTTGCTGGCCCTTTAA | ACT |
| 1385404 | CCTAAAGCAGCCACTGT | ACT |
| 1368716 | CAATGAGAAATGCCAGC | ACG |
| 4693316 | GCGATAGAGTGAGATTCTG | ACG |
| 1905707 | TGTAGTTTCCCGAGCTAGATT | ACT |
| 1905708 | AGATAGATGAATGGAGAACCC | ACT |
| 1905709 | GTTTCTCAGTGTCATCAAATA | CGT |
| 3912442 | AAAAAGACATATCTTCTTTTAGTG | CGT |
| 2082553 | CCACTCAGATATTCCATAAC | ACT |
| 6831638 | CCTTTACTGTGATATTTATTTCTT | ACG |
| 5860329 | GCTCCTGAAACATATTCATG | ACT |
| 2870702 | GAAGGCATTATGACATGAAT | ACG |
| 2870703 | TTGTTTATTCAAATCTGCCA | ACG |
| 1948016 | GCCAGTAATCTCTCCAATGAT | CGT |
| 6835836 | CACTCAGCTGAATAGACA | ACT |
| 1994253 | CTGACTCATACATCCTTTG | ACT |
| 1905710 | GAGCTATTCCAATGTGCT | CGT |
| 1485019 | AAGCTCCCCAAAAGGTGTTTAATT | CGT |
| 978191 | TGTACAAATCTGAGGGC | ACG |
| 1385405 | CCTGTGACAGGATTCCAGCA | CGT |
| 7694361 | GGATTACAGTAGTTTCCC | ACG |
| 1905711 | CCATATCCACAGGTTCT | ACT |
| 1905734 | CTTAGCCACTCTGATAATCT | ACT |
| 1485012 | ACACTGCAAAAAGCACT | ACT |
| 1485013 | TGCAGAGATAATGTATGTAGAA | ACT |
| 4692981 | TCAAACACAGTTTATATGAGATAA | CGT |
| 7670552 | CAACCATGCTGCTATGA | ACT |
| 7670932 | CATGCTAAGGGAAAGAAG | ACG |
| 7688091 | GGATGGGTGAATTGTATATTAT | ACT |
| 7440540 | TTAAGGGAGAGCATGAAA | ACT |
| 2171000 | GGTAGAAATGGACTTTGA | ACG |
| 2870704 | GCAGCTTCCTAACAAAAA | ACT |
| 7655758 | GCCTTTGAAAGAATCCAA | ACT |
| 7661436 | CTTTTTTGTTTCTATCCAGG | ACG |
| 7662289 | TGCTTCTTTATTCCCCA | ACT |
| 7667044 | GCTTTGTTTTTGATGAGTG | ACG |
| 7691929 | TTAATTCCTGAGACGTGT | ACT |
| 5860330 | CTTGCAAGTGATTAAAAAAAAAA | ACT |
| 901013 | GAAAGCATTCATCTCACTA | ACT |
| 901012 | GTAGTGTCTACAAAGGGTATA | ACT |
| 901011 | GTTGATCTCCTTCCTGG | ACT |
| 1948018 | GAGAAAGTAGACTTTCTCAT | ACT |
| 2870705 | CTTTCTCACATGCTTGC | ACT |
| 1948017 | CAGCAACCATTGAGGGTGAAT | ACG |
| 1905733 | TGTCCTTGACTGATTTTTAG | ACG |
| 1385408 | TCTAGGAAGTATGAGATGG | ACT |
| 1385409 | CAATAGTAACTGTCAACTGT | ACT |
| 1385410 | CAAGGAAGCTAGAGCCA | ACT |
| 1485026 | CTTCAATTAGAAAGCACCT | ACT |
| 1485027 | GTGCTGTGAAGACAAATTA | CGT |
| 2904483 | GGATTGTTTCTTCCTCT | CGT |
| 1385408 | TGTATACAGAAAAAAGCATGA | CGT |
| 1905732 | GTTGGTGCAAAAGTAACT | ACT |
| 2046418 | GTCTATCTCCCTGACAC | ACG |
| 2200377 | AAAAATTAAAACATTCACTGCTTA | ACT |
| 1905731 | TTCTGTAATCTAATCAAGATAGG | ACT |
| 1905730 | TCTGACAGGACTAAACAA | ACT |
| 975713 | GGTCACCTAAGGATTTTACA | ACT |
| 6820985 | AAAGGAGGCCTCTACCC | ACT |
| 7670441 | CATGCTCTTACATGCAAATA | ACT |
| 6810794 | CCACAAAACCAAGCTTATTA | CGT |
| 7676623 | TCTTCTTTAATGTGATGGTAC | ACT |
| 1154861 | GAATCCATAGATCGTATACTAAT | ACG |
| 1032125 | ATTTATATTCCGCCCCA | CGT |
| 1485022 | TCCAAAACGACCAGTCA | CGT |
| 1485024 | TTCATGCTAACTGATTATCAAAT | ACT |
| 3913651 | GGCAAAATTCCATGGCC | ACG |
| 4693319 | GAGTAATTGGACCTCTAC | ACT |
| 1872383 | CCATTCCATATCCTTACC | CGT |
| 2200376 | TTTTACATCTCACCCCA | CGT |
| 7668090 | GAATGCATTGGAGTGAG | ACT |
| 7692930 | ATCACACTCCGGGGACT | ACT |
| 967096 | AGGGCAACAGGGACACA | ACT |
| 6822249 | GTATTCACCATTGCAAAAA | CGT |
| 6532405 | GAACAACTGAAATCTGAAGTA | ACG |
| 1017897 | AACTGCAAATACACATTTCA | ACT |
| 7672674 | GTTTTGAACACTTAATGTTTG | ACT |
| 7694568 | GATTTCACCCCTTTCCC | ACG |
| 2904484 | AAATGTTCTCCAAGAAAGAT | ACT |
| 7340830 | GTTTACATATGAGGAAATGTAG | ACG |
| 1485033 | GCCCTCTAAAGACATGA | ACT |
| 2870706 | GAAGAAAATAGGCTGATTTTAT | ACT |
| 1905729 | CTAGCTCTTCAGAATTAATTGG | ACT |
| 4693320 | CCCACAAAGTCTTATGCA | ACT |
| 6848749 | CCAACAGAGAGAGGTATTTA | CGT |
| 6532406 | CATGGTCTACACACCTTTA | ACG |
| 6532407 | CCTTGTAAGACTACCTGAA | ACT |
| 1905728 | CAGTTAATAATTGTAGATCCATG | ACT |
| 6819866 | GCTCTTCTGTAAGAAGTCT | CGT |
| 1905727 | ATGATTGTAGATCATTTTGATGTA | ACT |
| 7674069 | TTCCTAACTCTTCACCTT | ACT |
| 1905724 | CATTGTTAGCAAGTGGAA | ACT |
| 1905723 | GAGCATAAAGATGCTCTCAGT | ACT |
| 1485020 | CTCCGTTATCTCCATGT | ACT |
| 6814101 | GCAAATGTAGTTGTATGTAATTT | ACT |

Genetic Analysis

Allelotyping results are shown for female cases and controls in Table 19. The allele frequency for the A2 allele is noted in the fifth and sixth columns for control pools and case pools, respectively, where “AF” is allele frequency. Some SNPs do not have an allele frequency disclosed because of failed assays.

TABLE 19

| | Position in | | | | | | | Low BMD |
|---|---|---|---|---|---|---|---|---|
| | SEQ ID | Chromosome | A1/A2 | Control AF | Case AF | | | Associated |
| dbSNP rs# | NO: 3 | Position | Allele | (High BMD) | (Low BMD) | p-Value | OR | Allele |
| 1433661 | 206 | 94744306 | T/C | T = 0.11 | T = 0.17 | 0.0089 | 1.64 | T |
| | | | | C = 0.89 | C = 0.83 | | | |
| 1485009 | 243 | 94744343 | A/G | A = 0.00 | A = 0.00 | | | |
| | | | | G = 1.00 | G = 1.00 | | | |
| 7681947 | 2467 | 94746567 | T/C | T = | T = 0.89 | | | |
| | | | | C = | C = 0.11 | | | |
| 1816432 | 3550 | 94747650 | A/G | A = 0.56 | A = 0.61 | 0.0779 | 1.23 | A |
| | | | | G = 0.44 | G = 0.39 | | | |
| 1485018 | 4994 | 94749094 | C/G | C = 1.00 | C = 0.99 | | | |
| | | | | G = 0.00 | G = 0.01 | | | |
| 1485017 | 5167 | 94749267 | G/T | G = | G = | | | |
| | | | | T = | T = | | | |
| 7438397 | 5193 | 94749293 | C/T | C = 0.94 | C = 0.94 | 0.8804 | 0.96 | C |
| | | | | T = 0.06 | T = 0.06 | | | |
| 6834311 | 5273 | 94749373 | G/A | G = 0.51 | G = 0.56 | 0.1212 | 1.20 | G |
| | | | | A = 0.49 | A = 0.44 | | | |
| 1368717 | 5733 | 94749833 | A/G | A = 0.10 | A = 0.13 | 0.1573 | 1.31 | A |
| | | | | G = 0.90 | G = 0.87 | | | |
| 1017391 | 7817 | 94751917 | A/C | A = 0.64 | A = 0.63 | 0.6402 | 0.94 | C |
| | | | | C = 0.36 | C = 0.37 | | | |
| 2870701 | 7818 | 94751918 | T/A | T = 0.00 | T = 0.00 | | | |
| | | | | A = 1.00 | A = 1.00 | | | |
| 7679839 | 8612 | 94752712 | T/G | T = 0.97 | T = 0.90 | 0.0000 | 0.31 | G |
| | | | | G = 0.03 | G = 0.10 | | | |
| 1385404 | 9158 | 94753258 | A/G | A = | A = 1.00 | | | |
| | | | | G = | G = 0.00 | | | |
| 1368716 | 9285 | 94753385 | G/A | G = 0.89 | G = 0.83 | 0.0251 | 0.62 | A |
| | | | | A = 0.11 | A = 0.17 | | | |
| 4693316 | 10680 | 94754780 | G/A | G = 0.57 | G = 0.53 | 0.2431 | 0.87 | A |
| | | | | A = 0.43 | A = 0.47 | | | |
| 1905707 | 11866 | 94755966 | T/C | T = 0.07 | T = 0.13 | 0.0008 | 1.99 | T |
| | | | | C = 0.93 | C = 0.87 | | | |
| 1905708 | 11958 | 94756058 | A/G | A = 0.97 | A = 0.92 | 0.0003 | 0.35 | G |
| | | | | G = 0.03 | G = 0.08 | | | |
| 1905709 | 12044 | 94756144 | A/T | A = 0.05 | A = 0.06 | 0.8882 | 1.08 | A |
| | | | | T = 0.95 | T = 0.94 | | | |
| 3912442 | 12753 | 94756853 | G/T | G = 0.00 | G = 0.00 | | | |
| | | | | T = 1.00 | T = 1.00 | | | |
| 2082553 | 15585 | 94759685 | A/G | A = 0.00 | A = 0.11 | | | |
| | | | | G = 1.00 | G = 0.89 | | | |
| 6831638 | 17299 | 94761399 | C/T | C = 1.00 | C = 1.00 | | | |
| | | | | T = 0.00 | T = 0.00 | | | |
| 5860329 | 18816 | 94762916 | —/G | — = 0.00 | — = 0.00 | | | |
| | | | | G = 1.00 | G = 1.00 | | | |
| 2870702 | 24022 | 94768122 | C/T | C = 0.66 | C = 0.65 | 0.8596 | 0.98 | T |
| | | | | T = 0.34 | T = 0.35 | | | |
| 2870703 | 24994 | 94769094 | C/T | C = 0.63 | C = 0.62 | 0.7613 | 0.96 | T |
| | | | | T = 0.37 | T = 0.38 | | | |
| 1948016 | 26637 | 94770737 | G/T | G = 0.52 | G = 0.47 | 0.1367 | 0.84 | T |
| | | | | T = 0.48 | T = 0.53 | | | |
| 6835836 | 27635 | 94771735 | C/G | C = 0.00 | C = 0.00 | | | |
| | | | | G = 1.00 | G = 1.00 | | | |
| 1994253 | 28773 | 94772873 | T/C | T = 0.92 | T = 0.87 | 0.0134 | 0.61 | C |
| | | | | C = 0.08 | C = 0.13 | | | |
| 1905710 | 29430 | 94773530 | A/T | A = 0.39 | A = 0.34 | 0.2784 | 0.83 | T |
| | | | | T = 0.61 | T = 0.66 | | | |
| 1485019 | 29876 | 94773976 | T/A | T = 0.14 | T = 0.19 | 0.0338 | 1.42 | T |
| | | | | A = 0.86 | A = 0.81 | | | |
| 978191 | 30364 | 94774464 | C/T | C = 0.00 | C = 0.00 | | | |
| | | | | T = 1.00 | T = 1.00 | | | |
| 1385405 | 31057 | 94775157 | C/A | C = 0.68 | C = 0.66 | 0.6519 | 0.94 | A |
| | | | | A = 0.32 | A = 0.34 | | | |
| 7694361 | 31782 | 94775882 | C/T | C = 0.21 | C = 0.24 | 0.1629 | 1.22 | C |
| | | | | T = 0.79 | T = 0.76 | | | |
| 1905711 | 33400 | 94777500 | A/C | A = 0.00 | A = 0.00 | | | |
| | | | | C = 1.00 | C = 1.00 | | | |
| 1905734 | 35588 | 94779688 | A/C | A = 0.92 | A = 0.88 | 0.0229 | 0.61 | C |
| | | | | C = 0.08 | C = 0.12 | | | |
| 1485012 | 37663 | 94781763 | C/G | C = 0.96 | C = 0.91 | 0.0032 | 0.47 | G |
| | | | | G = 0.04 | G = 0.09 | | | |
| 1485013 | 37865 | 94781965 | T/C | T = 0.00 | T = 0.00 | | | |
| | | | | C = 1.00 | C = 1.00 | | | |
| 4692981 | 38218 | 94782318 | A/T | A = 0.68 | A = 0.72 | 0.1741 | 1.19 | A |
| | | | | T = 0.32 | T = 0.28 | | | |
| 7670552 | 39375 | 94783475 | T/C | T = 0.11 | T = 0.22 | 0.0000 | 2.26 | T |
| | | | | C = 0.89 | C = 0.78 | | | |
| 7670932 | 39559 | 94783659 | C/T | C = 1.00 | C = 1.00 | | | |
| | | | | T = 0.00 | T = 0.00 | | | |
| 7688091 | 39833 | 94783933 | A/G | A = 0.00 | A = 0.13 | | | |
| | | | | G = 1.00 | G = 0.87 | | | |
| 7440540 | 40135 | 94784235 | A/G | A = 0.00 | A = 0.00 | | | |
| | | | | G = 1.00 | G = 1.00 | | | |
| 2171000 | 41698 | 94785798 | G/A | G = 0.00 | G = 0.00 | | | |
| | | | | A = 1.00 | A = 1.00 | | | |
| 2870704 | 42249 | 94786349 | T/C | T = 0.53 | T = 0.53 | 0.8418 | 1.02 | T |
| | | | | C = 0.47 | C = 0.47 | | | |
| 7655758 | 42571 | 94786671 | G/C | G = 0.21 | G = 0.25 | 0.0531 | 1.32 | G |
| | | | | C = 0.79 | C = 0.75 | | | |
| 7661436 | 42977 | 94787077 | G/A | G = 0.44 | G = 0.45 | 0.7243 | 1.04 | G |
| | | | | A = 0.56 | A = 0.55 | | | |
| 7662289 | 43548 | 94787648 | T/C | T = 0.24 | T = 0.29 | 0.1625 | 1.27 | T |
| | | | | C = 0.76 | C = 0.71 | | | |
| 7667044 | 43631 | 94787731 | G/A | G = 0.18 | G = 0.22 | 0.0669 | 1.31 | G |
| | | | | A = 0.82 | A = 0.78 | | | |
| 7691929 | 43705 | 94787805 | T/C | T = 0.93 | T = 0.90 | 0.0439 | 0.64 | C |
| | | | | C = 0.07 | C = 0.10 | | | |
| 5860330 | 43817 | 94787917 | T/— | T = 0.48 | T = 0.52 | 0.1449 | 1.19 | T |
| | | | | — = 0.52 | — = 0.48 | | | |
| 901013 | 44374 | 94788474 | A/C | A = 0.00 | A = 0.00 | | | |
| | | | | C = 1.00 | C = 1.00 | | | |
| 901012 | 44464 | 94788564 | A/C | A = 0.00 | A = 0.00 | | | |
| | | | | C = 1.00 | C = 1.00 | | | |
| 901011 | 44788 | 94788888 | T/C | T = 1.00 | T = 1.00 | | | |
| | | | | C = 0.00 | C = 0.00 | | | |
| 1948018 | 48962 | 94793062 | A/G | A = 0.10 | A = 0.19 | 0.0002 | 2.20 | A |
| | | | | G = 0.90 | G = 0.81 | | | |
| 2870705 | 48993 | 94793093 | T/G | T = 1.00 | T = 1.00 | | | |
| | | | | G = 0.00 | G = 0.00 | | | |
| 1948017 | 49110 | 94793210 | C/T | C = 0.12 | C = 0.17 | 0.0068 | 1.60 | C |
| | | | | T = 0.88 | T = 0.83 | | | |
| 1905733 | 49434 | 94793534 | C/T | C = 0.02 | C = 0.03 | 0.5881 | 1.41 | C |
| | | | | T = 0.98 | T = 0.97 | | | |
| 1385408 | 49523 | 94793623 | T/G | T = 1.00 | T = 1.00 | | | |
| | | | | G = 0.00 | G = 0.00 | | | |
| 1385409 | 49742 | 94793842 | C/G | C = 1.00 | C = 1.00 | | | |
| | | | | G = 0.00 | G = 0.00 | | | |
| 1385410 | 49907 | 94794007 | G/C | G = 0.53 | G = 0.51 | 0.5313 | 0.93 | C |
| | | | | C = 0.47 | C = 0.49 | | | |
| 1485026 | 50028 | 94794128 | G/C | G = 1.00 | G = 1.00 | | | |
| | | | | C = 0.00 | C = 0.00 | | | |
| 1485027 | 50089 | 94794189 | G/T | G = 0.00 | G = 0.00 | | | |
| | | | | T = 1.00 | T = 1.00 | | | |
| 2904483 | 51588 | 94795688 | C/A | C = 0.68 | C = 0.65 | 0.4125 | 0.90 | A |
| | | | | A = 0.32 | A = 0.35 | | | |
| 1385406 | 52899 | 94796999 | C/A | C = 0.00 | C = 0.00 | | | |
| | | | | A = 1.00 | A = 1.00 | | | |
| 1905732 | 54088 | 94798188 | A/C | A = 0.48 | A = 0.47 | 0.8078 | 0.97 | C |
| | | | | C = 0.52 | C = 0.53 | | | |
| 2046418 | 56538 | 94800638 | C/T | C = 0.64 | C = 0.65 | 0.9229 | 1.01 | C |
| | | | | T = 0.36 | T = 0.35 | | | |
| 2200377 | 59071 | 94803171 | T/C | T = 0.00 | T = 0.04 | | | |
| | | | | C = 1.00 | C = 0.96 | | | |
| 1905731 | 59110 | 94803210 | A/C | A = 1.00 | A = 1.00 | | | |
| | | | | C = 0.00 | C = 0.00 | | | |
| 1905730 | 59178 | 94803278 | A/G | A = 0.74 | A = 0.73 | 0.9270 | 0.99 | G |
| | | | | G = 0.26 | G = 0.27 | | | |
| 975713 | 61087 | 94805187 | T/C | T = 1.00 | T = 1.00 | | | |
| | | | | C = 0.00 | C = 0.00 | | | |
| 6820985 | 61300 | 94805400 | C/G | C = 0.99 | C = 0.99 | | | |
| | | | | G = 0.01 | G = 0.01 | | | |
| 7670441 | 62171 | 94806271 | T/C | T = 0.67 | T = 0.66 | 0.7167 | 0.96 | C |
| | | | | C = 0.33 | C = 0.34 | | | |
| 6810794 | 62783 | 94806883 | T/A | T = 1.00 | T = 1.00 | | | |
| | | | | A = 0.00 | A = 0.00 | | | |
| 7676623 | 62983 | 94807083 | T/C | T = 0.00 | T = 0.00 | | | |
| | | | | C = 1.00 | C = 1.00 | | | |
| 1154861 | 63908 | 94808008 | C/T | C = 0.73 | C = 0.70 | 0.2406 | 0.84 | T |
| | | | | T = 0.27 | T = 0.30 | | | |
| 1032125 | 64088 | 94808188 | G/T | G = 0.50 | G = 0.51 | 0.9342 | 1.01 | G |
| | | | | T = 0.50 | T = 0.49 | | | |
| 1485022 | 64941 | 94809041 | A/T | A = 1.00 | A = 0.98 | | | |
| | | | | T = 0.00 | T = 0.02 | | | |
| 1485024 | 65050 | 94809150 | A/G | A = 0.04 | A = 0.12 | 0.0000 | 3.18 | A |
| | | | | G = 0.96 | G = 0.88 | | | |
| 3913651 | 68953 | 94813053 | C/T | C = 0.40 | C = 0.41 | 0.6884 | 1.05 | C |
| | | | | T = 0.60 | T = 0.59 | | | |
| 4693319 | 70093 | 94814193 | T/C | T = 0.81 | T = 0.80 | 0.7260 | 0.95 | C |
| | | | | C = 0.19 | C = 0.20 | | | |
| 1872383 | 71308 | 94815408 | C/A | C = 0.35 | C = 0.38 | 0.4769 | 1.10 | C |
| | | | | A = 0.65 | A = 0.62 | | | |
| 2200376 | 73009 | 94817109 | T/A | T = 0.65 | T = 0.63 | 0.6117 | 0.92 | A |
| | | | | A = 0.35 | A = 0.37 | | | |
| 7668090 | 74002 | 94818102 | A/G | A = 0.61 | A = 0.59 | 0.4762 | 0.92 | G |
| | | | | G = 0.39 | G = 0.41 | | | |
| 7692930 | 74294 | 94818394 | T/C | T = 0.00 | T = 0.00 | | | |
| | | | | C = 1.00 | C = 1.00 | | | |
| 967096 | 74879 | 94818979 | G/C | G = 0.00 | G = 0.00 | | | |
| | | | | C = 1.00 | C = 1.00 | | | |
| 6822249 | 76936 | 94821036 | G/T | G = 0.20 | G = | | | |
| | | | | T = 0.80 | T = | | | |
| 6532405 | 77195 | 94821295 | G/A | G = 0.44 | G = 0.46 | 0.5246 | 1.08 | G |
| | | | | A = 0.56 | A = 0.54 | | | |
| 1017897 | 77683 | 94821783 | T/C | T = 0.75 | T = 0.72 | 0.3873 | 0.89 | C |
| | | | | C = 0.25 | C = 0.28 | | | |
| 7672674 | 78283 | 94822383 | T/C | T = 0.81 | T = 0.82 | 0.6661 | 1.07 | T |
| | | | | C = 0.19 | C = 0.18 | | | |
| 7694568 | 78331 | 94822431 | C/T | C = 0.82 | C = 0.91 | 0.0004 | 2.33 | C |
| | | | | T = 0.18 | T = 0.09 | | | |
| 2904484 | 79362 | 94823462 | G/C | G = 0.67 | G = 0.66 | 0.8007 | 0.97 | C |
| | | | | C = 0.33 | C = 0.34 | | | |
| 7340830 | 80357 | 94824457 | C/T | C = 1.00 | C = 1.00 | | | |
| | | | | T = 0.00 | T = 0.00 | | | |
| 1485033 | 80653 | 94824753 | T/C | T = 0.74 | T = 0.73 | 0.6809 | 0.95 | C |
| | | | | C = 0.26 | C = 0.27 | | | |
| 2870706 | 80840 | 94824940 | A/G | A = 0.65 | A = 0.65 | 0.9576 | 1.01 | A |
| | | | | G = 0.35 | G = 0.35 | | | |
| 1905729 | 83203 | 94827303 | A/G | A = 0.66 | A = 0.62 | 0.1186 | 0.82 | G |
| | | | | G = 0.34 | G = 0.38 | | | |
| 4693320 | 85405 | 94829505 | T/C | T = 0.67 | T = 0.73 | 0.0335 | 1.32 | T |
| | | | | C = 0.33 | C = 0.27 | | | |
| 6848749 | 86441 | 94830541 | G/T | G = 0.14 | G = 0.22 | 0.0016 | 1.68 | G |
| | | | | T = 0.86 | T = 0.78 | | | |
| 6532406 | 86967 | 94831067 | G/A | G = 0.98 | G = 0.95 | 0.0460 | 0.44 | A |
| | | | | A = 0.02 | A = 0.05 | | | |
| 6532407 | 87121 | 94831221 | T/C | T = 0.95 | T = 0.91 | 0.0301 | 0.57 | C |
| | | | | C = 0.05 | C = 0.09 | | | |
| 1905728 | 89617 | 94833717 | T/C | T = 0.00 | T = 0.00 | | | |
| | | | | C = 1.00 | C = 1.00 | | | |
| 6819866 | 90969 | 94835069 | T/A | T = 0.51 | T = 0.42 | 0.0157 | 0.70 | A |
| | | | | A = 0.49 | A = 0.58 | | | |
| 1905727 | 94249 | 94838349 | G/C | G = 0.06 | G = 0.09 | 0.1689 | 1.48 | G |
| | | | | C = 0.94 | C = 0.91 | | | |
| 7674069 | 95811 | 94839911 | T/G | T = 1.00 | T = 1.00 | | | |
| | | | | G = 0.00 | G = 0.00 | | | |
| 1905724 | 96690 | 94840790 | T/G | T = 0.06 | T = 0.07 | 0.3032 | 1.31 | T |
| | | | | G = 0.94 | G = 0.93 | | | |
| 1905723 | 96731 | 94840831 | A/G | A = 0.71 | A = 0.70 | 0.7768 | 0.96 | G |
| | | | | G = 0.29 | G = 0.30 | | | |
| 1485020 | 97267 | 94841367 | C/G | C = 1.00 | C = 1.00 | | | C |
| | | | | G = 0.00 | G = 0.00 | | | |
| 6814101 | 97414 | 94841514 | T/G | T = | T = 0.00 | | | G |
| | | | | G = | G = 1.00 | | | |

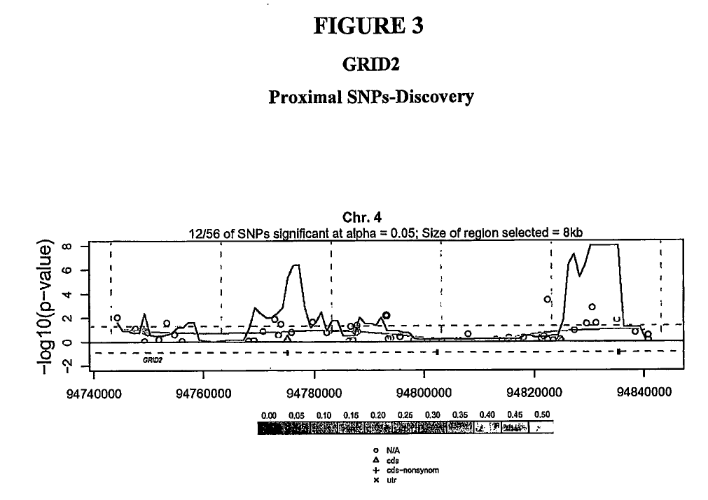

Allelotyping results were considered particularly significant with a calculated p-value of less than or equal to 0.05 for allelotype results. These values are indicated in bold. The allelotyping p-values were plotted in FIG. 3. The position of each SNP on the chromosome is presented on the x-axis. The y-axis gives the negative logarithm (base 10) of the p-value comparing the estimated allele in the case group to that of the control group. The minor allele frequency of the control group for each SNP designated by an X or other symbol on the graphs in FIG. 3 can be determined by consulting Table 19. For example, the left-most X on the left graph is at position 44917643. By proceeding down the Table from top to bottom and across the graphs from left to right the allele frequency associated with each symbol shown can be determined.

To aid the interpretation, multiple lines have been added to the graph. The broken horizontal lines are drawn at two common significance levels, 0.05 and 0.01. The vertical broken lines are drawn every 20 kb to assist in the interpretation of distances between SNPs. Two other lines are drawn to expose linear trends in the association of SNPs to the disease. The light gray line (or generally bottom-most curve) is a nonlinear smoother through the data points on the graph using a local polynomial regression method (W. S. Cleveland, E. Grosse and W. M. Shyu (1992) Local regression models. Chapter 8 of Statistical Models in S eds J. M. Chambers and T. J. Hastie, Wadsworth & Brook/Cole). The black line provides a local test for excess statistical significance to identify regions of association. This was created by use of a 10 kb sliding window with 1 kb step sizes. Within each window, a chi-square goodness of fit test was applied to compare the proportion of SNPs that were significant at a test wise level of 0.01, to the proportion that would be expected by chance alone (0.05 for the methods used here). Resulting p-values that were less than 10⁻⁸ were truncated at that value.

Finally, the exons and introns of the genes in the covered region are plotted below each graph at the appropriate chromosomal positions. The gene boundary is indicated by the broken horizontal line. The exon positions are shown as thick, unbroken bars. An arrow is place at the 3′ end of each gene to show the direction of transcription.

### Example 8

### PDE4D Proximal SNPs

It has been discovered that a polymorphic variation (rs1498608) in a gene encoding PDE4D is associated with the occurrence of low BMD (see Examples 1 and 2). One hundred sixteen additional allelic variants proximal to rs1498608 were identified and subsequently allelotyped in low BMD case and high BMD control sample sets as described in Examples 1 and 2. The polymorphic variants are set forth in Table 20. The chromosome position provided in column four of Table 20 is based on Genome “Build 34” of NCBI's GenBank.

TABLE 20

| | Position in | | | | | |
|---|---|---|---|---|---|---|
| | SEQ ID | | Chromosome | | | |
| dbSNP | NO: 4 | Chromosome | Position | Alleles (A1/A2) | genome_letter | deduced_iupac |
| 6886495 | 249 | 5 | 58309549 | g/c | g | S |
| 6450498 | 543 | 5 | 58309843 | a/t | a | W |
| 1472456 | 973 | 5 | 58310273 | g/a | c | Y |
| 4700315 | 1076 | 5 | 58310376 | a/g | a | R |
| 4700316 | 1276 | 5 | 58310576 | g/c | g | S |
| 7714708 | 1599 | 5 | 58310899 | a/g | a | R |
| 7710479 | 2755 | 5 | 58312055 | c/t | c | Y |
| 2968013 | 2911 | 5 | 58312211 | g/c | c | S |
| 2968014 | 4466 | 5 | 58313766 | a/g | a | R |
| 2968015 | 5754 | 5 | 58315054 | t/c | c | Y |
| 1391648 | 5762 | 5 | 58315062 | a/g | c | Y |
| 2055297 | 5967 | 5 | 58315267 | a/g | c | Y |
| 2055296 | 5972 | 5 | 58315272 | a/g | t | Y |
| 3989138 | 6390 | 5 | 58315690 | —/aa | a | N |
| 4700317 | 6984 | 5 | 58316284 | c/t | t | Y |
| 2036220 | 7234 | 5 | 58316534 | g/a | c | Y |
| 7727206 | 8196 | 5 | 58317496 | g/t | t | K |
| 7723432 | 8369 | 5 | 58317669 | a/g | a | R |
| 1546221 | 9565 | 5 | 58318865 | c/t | g | R |
| 4479801 | 11084 | 5 | 58320384 | c/t | c | Y |
| 4395595 | 11153 | 5 | 58320453 | c/t | c | Y |
| 4395596 | 11187 | 5 | 58320487 | t/c | c | Y |
| 4699932 | 11290 | 5 | 58320590 | g/a | a | R |
| 2936201 | 11386 | 5 | 58320686 | a/g | t | Y |
| 7356672 | 11441 | 5 | 58320741 | c/t | c | Y |
| 2936200 | 12373 | 5 | 58321673 | a/g | c | Y |
| 1909296 | 12602 | 5 | 58321902 | c/a | g | K |
| 7703131 | 13763 | 5 | 58323063 | c/g | c | S |
| 7445308 | 18697 | 5 | 58327997 | a/t | t | W |
| 3087748 | 18854 | 5 | 58328154 | t/c | g | R |
| 4321723 | 19107 | 5 | 58328407 | c/t | g | R |
| 2968016 | 19310 | 5 | 58328610 | c/t | c | Y |
| 5868151 | 20074 | 5 | 58329374 | c/— | c | N |
| 1874858 | 20145 | 5 | 58329445 | t/c | g | R |
| 1874857 | 20281 | 5 | 58329581 | a/c | t | K |
| 7712922 | 23117 | 5 | 58332417 | c/t | t | Y |
| 4631140 | 23585 | 5 | 58332885 | g/a | a | R |
| 4469166 | 23906 | 5 | 58333206 | t/c | t | Y |
| 1078369 | 24046 | 5 | 58333346 | c/g | g | S |
| 1078368 | 24450 | 5 | 58333750 | g/a | c | Y |
| 2968006 | 24619 | 5 | 58333919 | a/g | t | Y |
| 2968005 | 24637 | 5 | 58333937 | g/t | c | M |
| 2936190 | 24894 | 5 | 58334194 | c/t | c | Y |
| 2409613 | 25030 | 5 | 58334330 | g/c | g | S |
| 4415048 | 25732 | 5 | 58335032 | g/c | g | S |
| 2968004 | 27106 | 5 | 58336406 | a/c | g | K |
| 2968003 | 27395 | 5 | 58336695 | g/a | t | Y |
| 2968002 | 28971 | 5 | 58338271 | a/g | c | Y |
| 2936191 | 29755 | 5 | 58339055 | t/a | t | W |
| 1498610 | 30988 | 5 | 58340288 | a/g | g | R |
| 6874662 | 31827 | 5 | 58341127 | c/a | a | M |
| 3060393 | 31843 | 5 | 58341143 | a/aca | a | N |
| 7729722 | 32773 | 5 | 58342073 | g/a | a | R |
| 7733884 | 32787 | 5 | 58342087 | t/c | t | Y |
| 7714489 | 33099 | 5 | 58342399 | t/c | c | Y |
| 7735570 | 36854 | 5 | 58346154 | t/c | c | Y |
| 2936193 | 38026 | 5 | 58347326 | g/t | g | K |
| 2291851 | 38397 | 5 | 58347697 | t/c | c | Y |
| 2291852 | 38680 | 5 | 58347980 | a/g | a | R |
| 1498602 | 39626 | 5 | 58348926 | t/c | t | Y |
| 1995166 | 39682 | 5 | 58348982 | t/c | t | Y |
| 1498603 | 39710 | 5 | 58349010 | g/t | t | K |
| 1498604 | 39745 | 5 | 58349045 | c/a | a | M |
| 1498605 | 39901 | 5 | 58349201 | g/a | g | R |
| 1948651 | 39925 | 5 | 58349225 | c/t | t | Y |
| 4699934 | 40356 | 5 | 58349656 | g/t | t | K |
| 4700319 | 40393 | 5 | 58349693 | c/t | c | Y |
| 2279737 | 41230 | 5 | 58350530 | a/g | a | R |
| 7720361 | 41733 | 5 | 58351033 | c/t | c | Y |
| 7706419 | 41877 | 5 | 58351177 | g/t | g | K |
| 1006431 | 43555 | 5 | 58352855 | t/g | g | K |
| 1353747 | 44066 | 5 | 58353366 | g/t | t | K |
| 1498606 | 44134 | 5 | 58353434 | c/t | c | Y |
| 1353748 | 44181 | 5 | 58353481 | t/g | g | K |
| 1553113 | 45022 | 5 | 58354322 | a/c | a | M |
| 2968012 | 46856 | 5 | 58356156 | t/g | c | M |
| 2968011 | 48231 | 5 | 58357531 | c/a | t | K |
| 1498608 | 49652 | 5 | 58358952 | a/t | t | W |
| 2936189 | 50393 | 5 | 58359693 | t/a | a | W |
| 1498609 | 51103 | 5 | 58360403 | c/t | t | Y |
| 2968019 | 51733 | 5 | 58361033 | t/c | c | Y |
| 6891238 | 54733 | 5 | 58364033 | t/c | t | Y |
| 2968010 | 57173 | 5 | 58366473 | a/t | t | W |
| 2968009 | 58192 | 5 | 58367492 | c/t | g | R |
| 2936203 | 58506 | 5 | 58367806 | g/c | g | S |
| 1498601 | 59572 | 5 | 58368872 | g/a | t | Y |
| 1498600 | 59738 | 5 | 58369038 | a/g | t | Y |
| 1498599 | 61617 | 5 | 58370917 | g/a | t | Y |
| 2936202 | 63980 | 5 | 58373280 | c/g | c | S |
| 7730070 | 64161 | 5 | 58373461 | c/g | g | S |
| 6450501 | 66871 | 5 | 58376171 | a/g | g | R |
| 6450502 | 67063 | 5 | 58376363 | a/t | a | W |
| 6889456 | 67084 | 5 | 58376384 | a/g | a | R |
| 6894618 | 67477 | 5 | 58376777 | c/t | t | Y |
| 7706044 | 69282 | 5 | 58378582 | t/c | t | Y |
| 7707541 | 70363 | 5 | 58379663 | a/t | a | W |
| 7712076 | 70647 | 5 | 58379947 | c/t | t | Y |
| 6892860 | 71834 | 5 | 58381134 | c/t | c | Y |
| 6867053 | 72130 | 5 | 58381430 | c/g | c | S |
| 7737269 | 73495 | 5 | 58382795 | c/t | t | Y |
| 6864156 | 74542 | 5 | 58383842 | t/c | t | Y |
| 950447 | 75280 | 5 | 58384580 | t/c | a | R |
| 2936196 | 80740 | 5 | 58390040 | a/g | g | R |
| 7719347 | 82579 | 5 | 58391879 | t/c | t | Y |
| 1391649 | 82591 | 5 | 58391891 | c/a | a | M |
| 1391650 | 82976 | 5 | 58392276 | t/c | c | Y |
| 1391651 | 83040 | 5 | 58392340 | g/a | a | R |
| 1353749 | 85894 | 5 | 58395194 | g/a | a | R |
| 10682149 | 86020 | 5 | 58395320 | g/gcct | g | N |
| 5868153 | 86947 | 5 | 58396247 | g/ag | g | N |
| 1363882 | 88922 | 5 | 58398222 | c/g | g | S |
| 2409626 | 89662 | 5 | 58398962 | t/c | t | Y |
| 2968018 | 92367 | 5 | 58401667 | g/a | c | Y |
| 954740 | 93154 | 5 | 58402454 | a/g | c | Y |
| 986067 | 94979 | 5 | 58404279 | t/c | c | Y |
| 6869400 | 97598 | 5 | 58406898 | t/c | c | Y |
| 5010782 | 98532 | 5 | 58407832 | t/a | a | W |

Assay for Verifying and Allelotyping SNPs

The methods used to verify and allelotype the proximal SNPs of Table 20 are the same methods described in Examples 1 and 2 herein. The primers and probes used in these assays are provided in Table 21 and Table 22, respectively.

TABLE 21

| | | |
|---|---|---|
| DbSNP | | |
| rs# | Forward PCR primer | Reverse PCR primer |

| | | |
|---|---|---|
| 6886495 | AGTTTGCTTCCTGAACAATC | ATGCAGTTGAATCTCAATAC |
| 6450498 | GGACAGCTTTATGTTTAATAC | ACAAATACTCCATTGATGGT |
| 1472456 | GGGAGGTAAGGAATCATGAC | TTTTGAAGGACAAGTTCCCC |
| 4700315 | GGGAACTTGTCCTTCAAAAAG | CATTTTCTGTTACTCTGAGG |
| 4700316 | ATTGTGTCCATGCTTGGCAG | CCTCCTTCTATTGTGGAGAC |
| 7714708 | GCAACATACTAACTGGAATCC | GTTCTTCTGTTTGTCATGTGG |
| 7710479 | TGCTCCAAAAATGGCAATCG | ATTTCTACATCACCAGGGAG |
| 2968013 | CACCCCTAGATTTTGAAGGT | CAATGACACATTCCCTCACC |
| 2968014 | CCTTCCCTCCTTCATTCAAC | TGGATTCAGATTACGGGAGG |
| 2968015 | TGTCAGCTGTATGGTGAAGG | AACGCCAAGCCTATTTTCAG |
| 1391648 | AATTTGGTAAGACAGAGATG | TTGCTCTGTTCCTGAAAGAC |
| 2055297 | AAGCTTTGTTTCTCTTTCTC | AACGATTCTACATCTGCCCC |
| 2055296 | AAGCTTTGTTTCTCTTTCTC | AACGATTCTACATCTGCCCC |
| 3989138 | GCCTTCCCCCTCCTTTAAAA | CATTTCAGGATGAATACTTG |
| 4700317 | TTTTCCTGATATGATGAATG | CTAGTAATGCAGCAATAGTG |
| 2036220 | TGTATCCCAGGACATCTAGC | AACATTCAGGAAATTTCAGG |
| 7727206 | AAGGACTGGGTTTGCATTTC | CTAATCTTGGCAACATTCTG |
| 7723432 | TGAATCTAGGAGTGGATTTG | AGATCTGACCATGGTGATAG |
| 1546221 | GGAAAACTCCTTATGTTGGA | GCTGTATATTATATTACTGTG |
| 4479801 | ACTTCTAGAAGAAAACACAG | TATGCTTCATGCTTTTGTGG |
| 4395595 | AAACCACAAAAGCATGAAGC | TGGCTTGTTGTTCTGTTTTC |
| 4395596 | ATATTCCCTTCTCAAGTCTG | CTGGACTTTACCAAATTTCTC |
| 4699932 | CCATCTTTTCACTGGATTGTC | CAGACTTGAGAAGGGAATATG |
| 2936201 | GCATCTCACTGTGGTTTTAC | CACAATAAGAGGACAATCCAG |
| 7356672 | GACCATCATTAGACATTAGGG | TTAATTTGCTCTGCATCCAG |
| 2936200 | TCACAACTGGGAATTCACTG | GAGCTCCACCATTATTTCCC |
| 1909296 | CCGAGTAGCTGGGATTACAT | TGGTGAAACCCTGTCTCTAC |
| 7703131 | AGAGAAGAAGGCTTCAGACG | TTTAGCTTCTTTGCGATGGG |
| 7445308 | ATACAGCGGTTGGGACTATC | GCACATTGTGCACATGTACC |
| 3087748 | TAGTCCATAGGAATCTGCTG | GCTGCTGTACATTACAACAC |
| 4321723 | GAATTTTACTTGGAACCCTGG | CCTATCTTACTACTGAACTC |
| 2968016 | GTAAACCTTGGACTTATGGG | ACGATGTTACCCTCTTTTCC |
| 5868151 | TAGAATAGACTACATCCATC | GGATAAGGAAGTTTCTTAGG |
| 1874858 | GCCTAAGAAACTTCCTTATCC | ATATTGCCAACTAGGAGTAC |
| 1874857 | ATGAAGACTTTACTGAAGGC | CATACACTACTAACCTGTTGC |
| 7712922 | CTCTTTCTAAGGGCTTCTGG | AGAGGAGTCGGACTTTGTTC |
| 4631140 | TTGAAGTTGAGAGGGTCTCC | AAGCAAAGCACAAGCAACAG |
| 4469166 | TTAGGAGGGATGAGGAATGG | CTCCAATTGCACTGGGTTAC |
| 1078369 | TCCCTGAGCCTCTGTTTTCC | ATATGTCCCCACCACACTAC |
| 1078368 | ATGACTCATGGAGGCAACAG | GGAGAGCGATTTATGGATGG |
| 2968006 | CAGGGTTCATTTGGTGAAAC | ACTGACCTGTCTGGGATTTC |
| 2968005 | ACTGACCTGTCTGGGATTTC | CGCAGGGTTCATTTGGTGAA |
| 2936190 | AACAGACCTCCAACACAACC | TAGGATAAACTCACGGAAGG |
| 2409613 | AATAGGGTCAGTGGGATGAG | CCCACCTCCAAATCCTTTAC |
| 4415048 | CACCCAGCCTCAGAATATTT | TTCCCCACCAATACAACATC |
| 2968004 | CCCAAACATTATCTTCTGGC | GATCTCCATAAGGGTAAGTG |
| 2968003 | ACAAGAGGACAAGTGTTTAG | GGCCAGAGCTGTTCATAAAT |
| 2968002 | CCTTTGACTTTCCACAGAAC | ACACTCACTGGGTTGGGCTA |
| 2936191 | TGACCTTGATAACCTGGCTG | AGCTGTGCATATTGACTTCC |
| 1498610 | CATTAAATCACCACAGCAAC | TCCTTAGGCAGAATGGTC |
| 6874662 | ACTCTAGCCTGGGCAACAGA | ATGTCTTGTAGTTTCCAGTG |
| 3060393 | CCAGTGTATAGATCTTTCACC | AGAGCGAGACTCCATCACAA |
| 7729722 | GAGGAAAGTGTGTCTATTCAG | CCATACATCTGATAAGAGGC |
| 7733884 | TGTATCTCTGTTGGCCATTC | CCATACATCTGATAAGAGGC |
| 7714489 | AGGGTCTTGCTCTTTTGTTC | ATTGAGCCCGAGAATTTGAG |
| 7735570 | AGGATCTTCATAGAAGTGGC | AACTGAGGCTGTTTTCTCTC |
| 2936193 | CTTAGCATACAATGGGCACC | ACAGTGCCTACTATTGTCAG |
| 2291851 | GTTTCTTGCTTGTTGATGGC | TAGCCTTGGCCAAGAATTCA |
| 2291852 | ATCCTGATCTCTTACTAGTG | TTCTGTGTTCTCTCTTGAGG |
| 1498602 | CAGTAGTTTTTCAGCTAAATG | ATCCAAACATAGATCTCAAC |
| 1995166 | GCCACTTAGCAATGTGCAAG | GGGAAAGTAACTAGCTGCTC |
| 1498603 | GTGTGGTTGATTCTGTGTAAG | ACTACTGTCGAATCACCTTG |
| 1498604 | GCTTGCACATTGCTAAGTGG | CCATGGGCCTGAGTTCTTAA |
| 1498605 | CTGTGGCATTAGGCACCTTT | AGGACACTGGACATATTCAG |
| 1948651 | CAGATGAGAATAAAGGTGCC | TATTGCTCTCTTACTGGGAC |
| 4699934 | AGGACTGGTAATGTTGTGAG | GACCTTGTAAATAGGTGGCC |
| 4700319 | GACCTTGTAAATAGGTGGCC | GCTGAAGGATTCAGCCAGTA |
| 2279737 | AAAACCTCTCAATTTATTTC | ATGGCCTCCAACAAGGTAAG |
| 7720361 | ACTATGCGACTTTTCAACTG | ACCCCCTCGCCATCCGCTA |
| 7706419 | ACCCGGCCGCGGCTGATTCAT | GCCCCGCCTGCCGAGCCTT |
| 1006431 | CCTTGCCAGGTGAATTAAAG | CTATACAGAGCAGGTATTTT |
| 1353747 | TGAGAAAGTTGGAGTGCAGG | AATCATTGGTTACAATGAAG |
| 1498606 | ACTCCAACTTTCTCAAAGCC | TTGGTGAAGATGGAGGAAGG |
| 1353748 | CTCCATCTTCACCAAGTTCC | GAAATATAATGTGTGGAGCC |
| 1553113 | AGCCTTTAGGGAGTTTAGCC | GCACCAAATCTTGCTAAGTC |
| 2968012 | CAGTTCTATCTACTGTAGAC | CAGTTCCAGCTTCTCTTCTC |
| 2968011 | ATAATGGTTGCACTGACTTC | ACTGTGTGACATGGGATCTG |
| 1498608 | GAATCCCTGTTCATTCCTTG | ATAACCTCGGGGTCCAGAAA |
| 2936189 | TGATCTCAGACTTCCAGCTC | TTTGTCTTAGCTCAAGCTGC |
| 1498609 | TAGCTCAACTGTTCTTCAGG | CAGAGTGAGTGTAAATATAC |
| 2968019 | CCCAGGCCAGTATTACTGTT | TGACATTTACAGGCACTCAG |
| 6891238 | CCTTTATTCAGGCTGCAGAC | TGTGGTTTTAATGGCTGTGG |
| 2968010 | ATGTATGATTCACTCTGATG | CAAACCAATTGGTAGATTTC |
| 2968009 | GTAGGCAAAGATGAATCACG | TGTTTTACCTAGCAAGGAGC |
| 2936203 | AGTTCAGAGGTTCCAGACAG | AGCCAAGCTTGCAAACTCTG |
| 1498601 | TTGGTTAGATCCAGCTCTGC | TAAGAGGAACAGGGATCTGC |
| 1498600 | ATATAGGTACTGCTTTCTCC | CCTTGTTTCCAAATCTGAGC |
| 1498599 | GGAACATTTGGCTACATCATG | CCACAGAGCTGATTTAATTTC |
| 2936202 | TGTAAGAGGAGGGTGTGATG | TGACTCTGCAGGACTGTCTG |
| 7730070 | AAATATACTTGGGTAGAGAG | GATAGCTAACACATTTCTGAC |
| 6450501 | AGCTAGACTACATAGCCTCC | TGTAGGACATGACAGCAAAC |
| 6450502 | TAACAAGTCAAAACAGATGG | ATTTTCAATATTCTGCACAC |
| 6889456 | GGATCTCTATTAAACCTCTC | TTCAAGGGTTACTGATACTC |
| 6894618 | CCATCAACAGATGAATGGAC | TCCTTCCTTTTTTAGGGCTG |
| 7706044 | GACTTGGTATTTTGTGAGGG | TCCACCATGTGAGGACAAAG |
| 7707541 | TTTTGAGTCATGAAAAACTG | CACATGTATTAATTAAGTAGG |
| 7712076 | AAGCCATACCACAAGCATTC | GTTCACGTGAAATACACTACC |
| 6892860 | CAGAAGATGCAATGAAAAGAC | AGAACAAAATGTTAGGACGC |
| 6867053 | TTATCCGCATTCCACTCTTG | ACACTGGTCCTCATAACAAG |
| 7737269 | TTTAAAGGCTCGCACCTCAG | TCCGAAGGAAAGTGATTCTC |
| 6864156 | GTTGGATCATCAAAAAGTGG | GGCTAGAGTACTAAAAATCAG |
| 950447 | GTGAGTAGTCTGAATTTGTC | AGGCTTCAGAATCGGTCATC |
| 2936196 | CTCCAAAACATAGTAAGTGC | TGAACATTTAGAATTAGGGG |
| 7719347 | ACATTCTGAGGTCATGCAGG | CTCAGAGCCTGCATTATCTC |
| 1391649 | GCTAATGCTTTACTCTGGTC | CACACTCCAAAATGTGCAAC |
| 1391650 | TTCAGCTGATACTTGCTCCC | CTCTACCTACCAACATCGTC |
| 1391651 | TTGACGTGATAGAAGTTTGG | CTGAAAAGGGTGGCTTTCTC |
| 1353749 | TATAGAGCAAAAAGCCAAGG | CCACTTCTACCACATTCTTC |
| 10682149 | GGGAGAATATAACCATTAAGT | GAATTGCATTTTATCCAATC |
| 5868153 | CATAGGGACTATTTAAACTTC | GAGTGTTCTTTAGAGATTAAG |
| 1363882 | CTCACACAGGCAATGAGTAG | AGCCACTACTTCTCAATCTC |
| 2409626 | CCTGGTCTCAAGCAATCATC | ATAAGGCCAGATGTGGTGAC |
| 2968018 | GCAGAGAGATGAGAGGAAAC | CCTCATATCTAATCTCTCCC |
| 954740 | ATACTTGGGAGCACTCAACG | CAACAAGACGGAATCCAAAG |
| 986067 | CTACAAATTGCTTAAGCAGGG | TGATAGAGTAGAGAGACTCC |
| 6869400 | ACAAGATCGTTGAATGGTGG | GACTTTGTATACTGCCACTC |
| 5010782 | AATCTGTGGGAGTTAGTGGT | TTCCTCATCCTCATCTTCCC |

TABLE 22

| | | |
|---|---|---|
| dbSNP | Extend | Term |
| rs# | Primer | Mix |

| | | |
|---|---|---|
| 6886495 | CCTGAACAATCTTAAATGC | ACT |
| 6450498 | GTCTTATGCATTTTGAAGG | CGT |
| 1472456 | GGAATCATGACTACTTGGA | ACG |
| 4700315 | GAAACAAAGCAAACGAC | ACT |
| 4700316 | TGCTTGGCAGGCTTTTT | ACT |
| 7714708 | CAGTATAAGTAATTTGGCCC | ACT |
| 7710479 | GAGCATTTTAATTGCTTCC | ACG |
| 2968013 | AGCTGAGAGCAGCCATG | ACT |
| 2968014 | GAGCTGTTTCTTTCAGTTT | ACT |
| 2968015 | ATGGTGAAGGTGGAGGA | ACT |
| 1391648 | TCAGCTGTATGGTGAAG | ACT |
| 2055297 | GTGCCTGACATAGAGTAG | ACT |
| 2055296 | GGCAGTGCCTGACATAG | ACT |
| 3989138 | CCCCTCCTTTAAAAAAAAAAA | CGT |
| 4700317 | CAAATGTAACAAAGTGCCA | ACG |
| 2036220 | GCCTCCCTGGCCTCTGC | ACG |
| 7727206 | AATCCTGTCACCTATGG | CGT |
| 7723432 | GTGCCTTGAATTAAAGAATC | ACT |
| 1546221 | CTTATGTTGGAGAGGTC | ACG |
| 4479801 | TAGAAAGTAGTTGTGATCTTG | ACG |
| 4395595 | AAAAATGGTATACTGGACTTTA | ACG |
| 4395596 | CTGTGGCTTGTTGTTCT | ACT |
| 4699932 | CACTGGATTGTCCTCTTA | ACG |
| 2936201 | CCTAATGTCTAATGATGGTC | ACT |
| 7356672 | CACAGTGAGATGCCACA | ACG |
| 2936200 | CTTGGATAACTGTATGCCT | ACT |
| 1909296 | TTACATGAGTGCACCAC | CGT |
| 7703131 | GGTAATAAACTTTTCCGAG | ACT |
| 7445308 | GTTGGGACTATCTACTTTTTT | CGT |
| 3087748 | CATAGGAATCTGCTGCCTCAC | ACT |
| 4321723 | ACTTGGAACCCTGGTATTTT | ACG |
| 2968016 | GGGGCAAAAAGACAATAAA | ACG |
| 5868151 | GAATAGACTACATCCATCAAAATA | ACG |
| 1874858 | GAAACTTCCTTATCCAAGGCC | ACT |
| 1874857 | GCAATTAAAAGTGGTACAATAA | ACT |
| 7712922 | CAATAAAAGGAAAGAGACTTC | ACG |
| 4631140 | AGAGGGTCTCCTGTAGG | ACG |
| 4469166 | GAGGAATGGAACTATTTTTAAAC | ACT |
| 1078369 | CTCTGTTTTCCCTGTAAAT | ACT |
| 1078368 | GCAACAGGTGAAGCTGA | ACG |
| 2968006 | GACTGAACCTTTAGGAGA | ACT |
| 2968005 | TTCTCCTAAAGGTTCAGT | CGT |
| 2936190 | CCAACACAACCTCTTAA | ACG |
| 2409613 | CCAAGTGACTAAAGCAGAT | ACT |
| 4415048 | AAAACCTAATGCTCAGTTAAA | ACT |
| 2968004 | TCTGGCATAAAGTAAACTAATC | ACT |
| 2968003 | GGGGACTAGTCAACAAA | ACG |
| 2968002 | TCTGATGAGGTCTTCTAC | ACT |
| 2936191 | GGCTGAAGTAGTGATAGG | CGT |
| 1498610 | GAAGCACAGAAGGCAAC | ACT |
| 6874662 | CAGAGCGAGACTCCATC | CGT |
| 3060393 | GATTTGCTCCTAAGTTTTTT | ACT |
| 7729722 | GTGTCTATTCAGATCCTTTG | ACG |
| 7733884 | TTGAGGAAAGTGTGTCT | ACT |
| 7714489 | GGCTGGAGTGCAGTGGC | ACT |
| 7735570 | TCCACATTATGCAACTACA | ACT |
| 2936193 | AAAAGTCAGTGTAAGAGTTC | CGT |
| 2291851 | TGATGGCATTGAAGCAG | ACT |
| 2291852 | GTTTTTATTGTGCTTTTCAATCT | ACT |
| 1498602 | GAATTTGAGCAGCTAGTT | ACT |
| 1995166 | TGTGCAAGCATTAGTGTAT | ACT |
| 1498603 | GAGCAGAATCAAAAGCC | CGT |
| 1498604 | GCTCTTCATTTTCTTACACAG | CGT |
| 1498605 | AGGCACCTTTATTCTCAT | ACG |
| 1948651 | GAATAAAGGTGCCTAATGC | ACG |
| 4699934 | GTAATAGTTGCCTCGTC | CGT |
| 4700319 | TTACTAATTGTCTCACAACATTA | ACG |
| 2279737 | TCGCCGGCATGGGAATC | ACT |
| 7720361 | AGTCCCAGTCCAGGAGC | ACG |
| 7706419 | TTCATTCACTTCAAGTGC | CGT |
| 1008431 | GTGAATTAAAGTTTCAGATTGAA | ACT |
| 1353747 | GGAGAGGAGCCACAGAA | CGT |
| 1498606 | CAAGTGATTTCCCAGTAG | ACG |
| 1353748 | ACCAAGTTCCCAAAGCT | ACT |
| 1553113 | GGAGTTTAGCCAATAGTTTTTGC | ACT |
| 2968012 | GACAGTATCAATTCCTTCC | ACT |
| 2968011 | TGACTTCAACTTTACTTTCTT | CGT |
| 1498608 | CCCTAAAAACTGTTCCAGGTA | CGT |
| 2936189 | CCAGAACTGTGAGAAATAAA | CGT |
| 1498609 | GTTTTCTATTTGAAATTGAGGTAT | ACG |
| 2968019 | CCAGTATTACTGTTTGAATCTC | ACT |
| 6891238 | CTGCAGACATTTCCTAC | ACT |
| 2968010 | TTCACTCTGATGTTTTCTATTT | CGT |
| 2968009 | CATAACCTCTGGTTTCC | ACG |
| 2936203 | GGAGGATATCCATGCCCC | ACT |
| 1498601 | AGCTCTGCCTCCCCCATTT | ACG |
| 1498600 | AGGTGCCAAGCTAGTCA | ACT |
| 1498599 | GGCTACATCATGTTTTGG | ACG |
| 2936202 | CCCTGGTAACTGTAGTG | ACT |
| 7730070 | TAGGCATGCTCAAACTC | ACT |
| 6450501 | GCCTCCTAAAAAAGCAC | ACT |
| 6450502 | GTTTAATGACATTTTAGAGAGG | CGT |
| 6889456 | AACCTCTCTAAAATGTCATTAA | ACT |
| 6894618 | GGACAAAGGAAATGTGATATATA | ACG |
| 7706044 | GGCTCATTTCCTGGGTC | ACT |
| 7707541 | GAAAAACTGACAACATATGAG | CGT |
| 7712076 | CCCAGAGGATATAGATTTCAT | ACG |
| 6892860 | GGATATTTCAGTGGTGCT | ACG |
| 6867053 | CACCATTAACCTGGTCTA | ACT |
| 7737269 | ACTCTTGATCAATAGTTTGG | ACG |
| 6864156 | GTAGTTGACAAATCTTACCT | ACT |
| 950447 | TTGTTAGTGTATTAGCCATTG | ACT |
| 2936196 | GTGCAAAGATCAATCACA | ACT |
| 7719347 | GGTCATGCAGGAAATAG | ACT |
| 1391649 | TCTGGTCTATTTCCTGC | CGT |
| 1391650 | CCCACAGGTTTTTCACA | ACT |
| 1391651 | GGGCCCCCGTTTTGCCCTG | ACG |
| 1353749 | CAAAAAGCCAAGGATATAAAATA | ACG |
| 10682149 | GTATGATCTGATTTCCATAAATAG | ACT |
| 5868153 | GAAATGAATAAATGGCAAAAAA | ACT |
| 1363882 | GCAATGAGTAGACTAAAAAAA | ACT |
| 2409626 | AGCAATCATCCCACCTC | ACT |
| 2968018 | CAGACACCCACTGACCA | ACG |
| 954740 | ACGAAGGTCCCGTCTGA | ACT |
| 986067 | CAGGGATTTAAAATGAAACC | ACT |
| 6869400 | AATGGTGGGTCATTAGTT | ACT |
| 5010782 | GCACCTACAGAGGAAAT | CGT |

Genetic Analysis

Allelotyping results are shown for female cases and controls in Table 23. The allele frequency for the A2 allele is noted in the fifth and sixth columns for control pools and case pools, respectively, where “AF” is allele frequency. Some SNPs do not have an allele frequency disclosed because of failed assays.

TABLE 23

| | Position in | | | | | | | Low BMD |
|---|---|---|---|---|---|---|---|---|
| dbSNP | SEQ ID | Chromosome | A1/A2 | Control AF | Case AF | | | Associated |
| rs# | NO: 4 | Position | Allele | (High BMD) | (Low BMD) | p-Value | OR | Allele |
| 6886495 | 249 | 58309549 | G/C | G = | G = 0.87 | | | |
| | | | | C = | C = 0.13 | | | |
| 6450498 | 543 | 58309843 | A/T | A = 0.81 | A = 0.85 | 0.1780 | 1.29 | A |
| | | | | T = 0.19 | T = 0.15 | | | |
| 1472456 | 973 | 58310273 | G/A | G = 1.00 | G = 1.00 | | | |
| | | | | A = 0.00 | A = 0.00 | | | |
| 4700315 | 1076 | 58310376 | A/G | A = | A = 0.95 | | | |
| | | | | G = | G = 0.05 | | | |
| 4700316 | 1276 | 58310576 | G/C | G = 0.87 | G = 0.84 | 0.1520 | 0.75 | C |
| | | | | C = 0.13 | C = 0.16 | | | |
| 7714708 | 1599 | 58310899 | A/G | A = 0.35 | A = 0.46 | 0.0013 | 1.60 | A |
| | | | | G = 0.65 | G = 0.54 | | | |
| 7710479 | 2755 | 58312055 | C/T | C = 1.00 | C = 1.00 | | | |
| | | | | T = 0.00 | T = 0.00 | | | |
| 2968013 | 2911 | 58312211 | G/C | G = 0.84 | G = 0.80 | 0.1256 | 0.79 | C |
| | | | | C = 0.16 | C = 0.20 | | | |
| 2968014 | 4466 | 58313766 | A/G | A = 0.22 | A = 0.25 | 0.2834 | 1.16 | A |
| | | | | G = 0.78 | G = 0.75 | | | |
| 2968015 | 5754 | 58315054 | T/C | T = 0.88 | T = 0.86 | 0.2038 | 0.80 | C |
| | | | | C = 0.12 | C = 0.14 | | | |
| 1391648 | 5762 | 58315062 | A/G | A = 0.25 | A = 0.24 | 0.8566 | 0.97 | G |
| | | | | G = 0.75 | G = 0.76 | | | |
| 2055297 | 5967 | 58315267 | A/G | A = 0.00 | A = 0.00 | | | |
| | | | | G = 1.00 | G = 1.00 | | | |
| 2055296 | 5972 | 58315272 | A/G | A = 0.94 | A = 0.94 | 0.8857 | 1.04 | A |
| | | | | G = 0.06 | G = 0.06 | | | |
| 3989138 | 6390 | 58315690 | —/AA | — = 0.53 | — = 0.50 | 0.3703 | 0.90 | A |
| | | | | AA = 0.47 | AA = 0.50 | | | |
| 4700317 | 6984 | 58316284 | C/T | C = 0.00 | C = 0.00 | | | |
| | | | | T = 1.00 | T = 1.00 | | | |
| 2036220 | 7234 | 58316534 | G/A | G = 0.44 | G = 0.44 | 0.9272 | 1.01 | G |
| | | | | A = 0.56 | A = 0.56 | | | |
| 7727206 | 8196 | 58317496 | G/T | G = 0.27 | G = 0.25 | 0.3954 | 0.89 | T |
| | | | | T = 0.73 | T = 0.75 | | | |
| 7723432 | 8369 | 58317669 | A/G | A = 0.82 | A = 0.82 | 0.7671 | 1.05 | A |
| | | | | G = 0.18 | G = 0.18 | | | |
| 1546221 | 9565 | 58318865 | C/T | C = 0.83 | C = 0.84 | 0.7282 | 1.06 | C |
| | | | | T = 0.17 | T = 0.16 | | | |
| 4479801 | 11084 | 58320384 | C/T | C = 0.86 | C = 0.87 | 0.7440 | 1.07 | C |
| | | | | T = 0.14 | T = 0.13 | | | |
| 4395595 | 11153 | 58320453 | C/T | C = 0.93 | C = 0.93 | 0.7241 | 1.09 | C |
| | | | | T = 0.07 | T = 0.07 | | | |
| 4395596 | 11187 | 58320487 | T/C | T = 0.01 | T = 0.02 | | | |
| | | | | C = 0.99 | C = 0.98 | | | |
| 4699932 | 11290 | 58320590 | G/A | G = 0.13 | G = 0.10 | 0.1440 | 0.76 | A |
| | | | | A = 0.87 | A = 0.90 | | | |
| 2936201 | 11386 | 58320686 | A/G | A = 0.17 | A = 0.19 | 0.3609 | 1.15 | A |
| | | | | G = 0.83 | G = 0.81 | | | |
| 7356672 | 11441 | 58320741 | C/T | C = 0.88 | C = 0.89 | 0.4757 | 1.17 | C |
| | | | | T = 0.12 | T = 0.11 | | | |
| 2936200 | 12373 | 58321673 | A/G | A = 1.00 | A = 1.00 | | | |
| | | | | G = 0.00 | G = 0.00 | | | |
| 1909296 | 12602 | 58321902 | C/A | C = 0.98 | C = 0.99 | | | |
| | | | | A = 0.02 | A = 0.01 | | | |
| 7703131 | 13763 | 58323063 | C/G | C = | C = | | | |
| | | | | G = | G = | | | |
| 7445308 | 18697 | 58327997 | A/T | A = 0.00 | A = 0.00 | | | |
| | | | | T = 1.00 | T = 1.00 | | | |
| 3087748 | 18854 | 58328154 | T/C | T = | T = 0.21 | | | |
| | | | | C = | C = 0.79 | | | |
| 4321723 | 19107 | 58328407 | C/T | C = 0.19 | C = 0.24 | 0.1033 | 1.30 | C |
| | | | | T = 0.81 | T = 0.76 | | | |
| 2968016 | 19310 | 58328610 | C/T | C = 0.45 | C = 0.44 | 0.7959 | 0.96 | T |
| | | | | T = 0.55 | T = 0.56 | | | |
| 5868151 | 20074 | 58329374 | C/— | C = 0.44 | C = 0.43 | 0.6659 | 0.95 | — |
| | | | | — = 0.56 | — = 0.57 | | | |
| 1874858 | 20145 | 58329445 | T/C | T = 0.12 | T = 0.17 | 0.1558 | 1.43 | T |
| | | | | C = 0.88 | C = 0.83 | | | |
| 1874857 | 20281 | 58329581 | A/C | A = 0.96 | A = 0.96 | 0.5485 | 0.83 | A |
| | | | | C = 0.04 | C = 0.04 | | | |
| 7712922 | 23117 | 58332417 | C/T | C = 0.05 | C = 0.00 | | | |
| | | | | T = 0.95 | T = 1.00 | | | |
| 4631140 | 23585 | 58332885 | G/A | G = 0.10 | G = 0.09 | 0.4903 | 0.87 | A |
| | | | | A = 0.90 | A = 0.91 | | | |
| 4469166 | 23906 | 58333206 | T/C | T = 0.97 | T = 0.98 | 0.6510 | 1.30 | T |
| | | | | C = 0.03 | C = 0.02 | | | |
| 1078369 | 24046 | 58333346 | C/G | C = 0.88 | C = 0.88 | 0.8279 | 0.96 | C |
| | | | | G = 0.12 | G = 0.12 | | | |
| 1078368 | 24450 | 58333750 | G/A | G = 0.50 | G = 0.47 | 0.3033 | 0.89 | A |
| | | | | A = 0.50 | A = 0.53 | | | |
| 2968006 | 24619 | 58333919 | A/G | A = 0.34 | A = 0.35 | 0.5216 | 1.08 | A |
| | | | | G = 0.66 | G = 0.65 | | | |
| 2968005 | 24637 | 58333937 | G/T | G = 0.37 | G = 0.36 | 0.7332 | 0.96 | T |
| | | | | T = 0.63 | T = 0.64 | | | |
| 2936190 | 24894 | 58334194 | C/T | C = 0.22 | C = 0.22 | 0.9071 | 1.02 | C |
| | | | | T = 0.78 | T = 0.78 | | | |
| 2409613 | 25030 | 58334330 | G/C | G = 1.00 | G = 1.00 | | | |
| | | | | C = 0.00 | C = 0.00 | | | |
| 4415048 | 25732 | 58335032 | G/C | G = 0.94 | G = 0.96 | 0.2666 | 1.36 | G |
| | | | | C = 0.06 | C = 0.04 | | | |
| 2968004 | 27106 | 58336406 | A/C | A = 0.92 | A = 0.88 | 0.1210 | 0.70 | C |
| | | | | C = 0.08 | C = 0.12 | | | |
| 2968003 | 27395 | 58336695 | G/A | G = 0.61 | G = 0.66 | 0.2364 | 1.23 | G |
| | | | | A = 0.39 | A = 0.34 | | | |
| 2968002 | 28971 | 58338271 | A/G | A = 0.81 | A = 0.81 | 0.8196 | 1.04 | A |
| | | | | G = 0.19 | G = 0.19 | | | |
| 2936191 | 29755 | 58339055 | T/A | T = 1.00 | T = 1.00 | | | |
| | | | | A = 0.00 | A = 0.00 | | | |
| 1498610 | 30988 | 58340288 | A/G | A = 0.00 | A = 0.00 | | | |
| | | | | G = 1.00 | G = 1.00 | | | |
| 6874662 | 31827 | 58341127 | C/A | C = 0.00 | C = 0.00 | | | |
| | | | | A = 1.00 | A = 1.00 | | | |
| 3060393 | 31843 | 58341143 | —/CA | — = 1.00 | — = 1.00 | | | |
| | | | | CA = 0.00 | CA = 0.00 | | | |
| 7729722 | 32773 | 58342073 | G/A | G = 0.01 | G = 0.01 | | | |
| | | | | A = 0.99 | A = 0.99 | | | |
| 7733884 | 32787 | 58342087 | T/C | T = 0.96 | T = 0.97 | 0.5282 | 1.26 | T |
| | | | | C = 0.04 | C = 0.03 | | | |
| 7714489 | 33099 | 58342399 | T/C | T = 0.15 | T = 0.14 | 0.7668 | 0.95 | C |
| | | | | C = 0.85 | C = 0.86 | | | |
| 7735570 | 36854 | 58346154 | T/C | T = 0.08 | T = 0.09 | 0.4593 | 1.18 | T |
| | | | | C = 0.92 | C = 0.91 | | | |
| 2936193 | 38026 | 58347326 | G/T | G = 0.25 | G = 0.28 | 0.3638 | 1.14 | G |
| | | | | T = 0.75 | T = 0.72 | | | |
| 2291851 | 38397 | 58347697 | T/C | T = 0.05 | T = 0.06 | 0.2114 | 1.40 | T |
| | | | | C = 0.95 | C = 0.94 | | | |
| 2291852 | 38680 | 58347980 | A/G | A = 0.98 | A = 0.98 | | | |
| | | | | G = 0.02 | G = 0.02 | | | |
| 1498602 | 39626 | 58348926 | T/C | T = 0.67 | T = 0.61 | 0.0496 | 0.77 | C |
| | | | | C = 0.33 | C = 0.39 | | | |
| 1995166 | 39682 | 58348982 | T/C | T = 0.54 | T = 0.55 | 0.7840 | 1.03 | T |
| | | | | C = 0.46 | C = 0.45 | | | |
| 1498603 | 39710 | 58349010 | G/T | G = 0.03 | G = 0.02 | | | |
| | | | | T = 0.97 | T = 0.98 | | | |
| 1498604 | 39745 | 58349045 | C/A | C = 0.16 | C = 0.13 | 0.2329 | 0.79 | A |
| | | | | A = 0.84 | A = 0.87 | | | |
| 1498605 | 39901 | 58349201 | G/A | G = 0.88 | G = 0.89 | 0.3713 | 1.18 | G |
| | | | | A = 0.12 | A = 0.11 | | | |
| 1948651 | 39925 | 58349225 | C/T | C = 0.58 | C = 0.56 | 0.5199 | 0.93 | T |
| | | | | T = 0.42 | T = 0.44 | | | |
| 4699934 | 40356 | 58349656 | G/T | G = 0.13 | G = 0.05 | 0.0043 | 0.35 | T |
| | | | | T = 0.87 | T = 0.95 | | | |
| 4700319 | 40393 | 58349693 | C/T | C = | C = 0.98 | | | |
| | | | | T = | T = 0.02 | | | |
| 2279737 | 41230 | 58350530 | A/G | A = 0.82 | A = 0.80 | 0.3520 | 0.87 | G |
| | | | | G = 0.18 | G = 0.20 | | | |
| 7720361 | 41733 | 58351033 | C/T | C = 1.00 | C = 1.00 | | | |
| | | | | T = 0.00 | T = 0.00 | | | |
| 7706419 | 41877 | 58351177 | G/T | G = 0.89 | G = 0.92 | 0.1509 | 1.38 | G |
| | | | | T = 0.11 | T = 0.08 | | | |
| 1006431 | 43555 | 58352855 | T/G | T = 0.22 | T = 0.30 | 0.0077 | 1.49 | T |
| | | | | G = 0.78 | G = 0.70 | | | |
| 1353747 | 44066 | 58353366 | G/T | G = 0.23 | G = 0.15 | 0.0025 | 0.61 | T |
| | | | | T = 0.77 | T = 0.85 | | | |
| 1498606 | 44134 | 58353434 | C/T | C = 0.85 | C = 0.87 | 0.3475 | 1.17 | C |
| | | | | T = 0.15 | T = 0.13 | | | |
| 1353748 | 44181 | 58353481 | T/G | T = 0.65 | T = 0.66 | 0.8645 | 1.02 | T |
| | | | | G = 0.35 | G = 0.34 | | | |
| 1553113 | 45022 | 58354322 | A/C | A = 0.95 | A = 0.97 | 0.1730 | 1.54 | A |
| | | | | C = 0.05 | C = 0.03 | | | |
| 2968012 | 46856 | 58356156 | T/G | T = 0.66 | T = 0.66 | 0.9069 | 1.01 | T |
| | | | | G = 0.34 | G = 0.34 | | | |
| 2968011 | 48231 | 58357531 | C/A | C = 0.67 | C = | | | |
| | | | | A = 0.33 | A = | | | |
| 1498608 | 49652 | 58358952 | A/T | A = 0.19 | A = 0.13 | 0.0118 | 0.64 | T |
| | | | | T = 0.81 | T = 0.87 | | | |
| 2936189 | 50393 | 58359693 | T/A | T = 0.63 | T = 0.65 | 0.6021 | 1.08 | T |
| | | | | A = 0.37 | A = 0.35 | | | |
| 1498609 | 51103 | 58360403 | C/T | C = 0.34 | C = 0.40 | 0.0314 | 1.30 | C |
| | | | | T = 0.66 | T = 0.60 | | | |
| 2968019 | 51733 | 58361033 | T/C | T = 0.62 | T = 0.64 | 0.5417 | 1.08 | T |
| | | | | C = 0.38 | C = 0.36 | | | |
| 6891238 | 54733 | 58364033 | T/C | T = 1.00 | T = 1.00 | | | |
| | | | | C = 0.00 | C = 0.00 | | | |
| 2968010 | 57173 | 58366473 | A/T | A = 0.90 | A = 0.95 | 0.0012 | 2.28 | A |
| | | | | T = 0.10 | T = 0.05 | | | |
| 2968009 | 58192 | 58367492 | C/T | C = 0.65 | C = 0.70 | 0.0959 | 1.24 | C |
| | | | | T = 0.35 | T = 0.30 | | | |
| 2936203 | 58506 | 58367806 | G/C | G = 0.17 | G = 0.14 | 0.1730 | 0.80 | C |
| | | | | C = 0.83 | C = 0.86 | | | |
| 1498601 | 59572 | 58368872 | G/A | G = 0.81 | G = 0.78 | 0.3899 | 0.83 | A |
| | | | | A = 0.19 | A = 0.22 | | | |
| 1498600 | 59738 | 58369038 | A/G | A = 0.48 | A = 0.46 | 0.5991 | 0.94 | G |
| | | | | G = 0.52 | G = 0.54 | | | |
| 1498599 | 61617 | 58370917 | G/A | G = 0.69 | G = 0.69 | 0.9739 | 1.00 | G |
| | | | | A = 0.31 | A = 0.31 | | | |
| 2936202 | 63980 | 58373280 | C/G | C = 0.20 | C = 0.14 | 0.0286 | 0.68 | G |
| | | | | G = 0.80 | G = 0.86 | | | |
| 7730070 | 64161 | 58373461 | C/G | C = 0.74 | C = 0.73 | 0.5744 | 0.93 | G |
| | | | | G = 0.26 | G = 0.27 | | | |
| 6450501 | 66871 | 58376171 | A/G | A = 0.00 | A = 0.09 | | | |
| | | | | G = 1.00 | G = 0.91 | | | |
| 6450502 | 67063 | 58376363 | A/T | A = 1.00 | A = 1.00 | | | |
| | | | | T = 0.00 | T = 0.00 | | | |
| 6889456 | 67084 | 58376384 | A/G | A = | A = 1.00 | | | |
| | | | | G = | G = 0.00 | | | |
| 6894618 | 67477 | 58376777 | C/T | C = 0.69 | C = 0.73 | 0.1542 | 1.21 | C |
| | | | | T = 0.31 | T = 0.27 | | | |
| 7706044 | 69282 | 58378582 | T/C | T = | T = 1.00 | | | |
| | | | | C = | C = 0.00 | | | |
| 7707541 | 70363 | 58379663 | A/T | A = 0.63 | A = 0.61 | 0.5874 | 0.93 | T |
| | | | | T = 0.37 | T = 0.39 | | | |
| 7712076 | 70647 | 58379947 | C/T | C = 0.00 | C = 0.00 | | | |
| | | | | T = 1.00 | T = 1.00 | | | |
| 6892860 | 71834 | 58381134 | C/T | C = 0.81 | C = 0.83 | 0.4091 | 1.14 | C |
| | | | | T = 0.19 | T = 0.17 | | | |
| 6867053 | 72130 | 58381430 | C/G | C = 0.46 | C = 0.44 | 0.4563 | 0.91 | G |
| | | | | G = 0.54 | G = 0.56 | | | |
| 7737269 | 73495 | 58382795 | C/T | C = 0.00 | C = 0.00 | | | |
| | | | | T = 1.00 | T = 1.00 | | | |
| 6864156 | 74542 | 58383842 | T/C | T = 0.99 | T = 0.99 | | | |
| | | | | C = 0.01 | C = 0.01 | | | |
| 950447 | 75280 | 58384580 | T/C | T = | T = 1.00 | | | |
| | | | | C = | C = 0.00 | | | |
| 2936196 | 80740 | 58390040 | A/G | A = 0.00 | A = 0.00 | | | |
| | | | | G = 1.00 | G = 1.00 | | | |
| 7719347 | 82579 | 58391879 | T/C | T = 1.00 | T = 1.00 | | | |
| | | | | C = 0.00 | C = 0.00 | | | |
| 1391649 | 82591 | 58391891 | C/A | C = 0.42 | C = 0.35 | 0.0162 | 0.74 | A |
| | | | | A = 0.58 | A = 0.65 | | | |
| 1391650 | 82976 | 58392276 | T/C | T = 0.75 | T = 0.75 | 0.9568 | 0.99 | T |
| | | | | C = 0.25 | C = 0.25 | | | |
| 1391651 | 83040 | 58392340 | G/A | G = 0.58 | G = 0.59 | 0.8831 | 1.02 | G |
| | | | | A = 0.42 | A = 0.41 | | | |
| 1353749 | 85894 | 58395194 | G/A | G = 0.43 | G = 0.39 | 0.1596 | 0.85 | A |
| | | | | A = 0.57 | A = 0.61 | | | |
| 10682149 | 86020 | 58395320 | —/CCT | — = 0.37 | — = 0.39 | 0.5382 | 1.08 | — |
| | | | | CCT = 0.63 | CCT = 0.61 | | | |
| 5868153 | 86947 | 58396247 | A/— | A = 0.72 | A = 0.71 | 0.7470 | 0.96 | — |
| | | | | — = 0.28 | — = 0.29 | | | |
| 1363882 | 88922 | 58398222 | C/G | C = 0.45 | C = 0.43 | 0.5228 | 0.93 | G |
| | | | | G = 0.55 | G = 0.57 | | | |
| 2409626 | 89662 | 58398962 | T/C | T = 0.84 | T = 0.85 | 0.8581 | 1.03 | T |
| | | | | C = 0.16 | C = 0.15 | | | |
| 2968018 | 92367 | 58401667 | G/A | G = 0.75 | G = 0.75 | 0.8411 | 0.97 | G |
| | | | | A = 0.25 | A = 0.25 | | | |
| 954740 | 93154 | 58402454 | A/G | A = 0.00 | A = 0.00 | | | |
| | | | | G = 1.00 | G = 1.00 | | | |
| 986067 | 94979 | 58404279 | T/C | T = 0.04 | T = 0.06 | 0.2475 | 1.43 | T |
| | | | | C = 0.96 | C = 0.94 | | | |
| 6869400 | 97598 | 58406898 | T/C | T = 0.00 | T = 0.00 | | | |
| | | | | C = 1.00 | C = 1.00 | | | |
| 5010782 | 98532 | 58407832 | T/A | T = 0.68 | T = 0.69 | 0.8644 | 1.02 | T |
| | | | | A = 0.32 | A = 0.31 | | | |

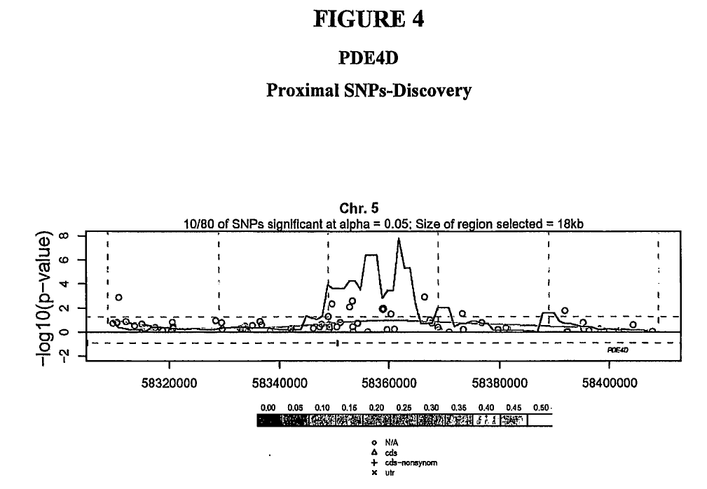

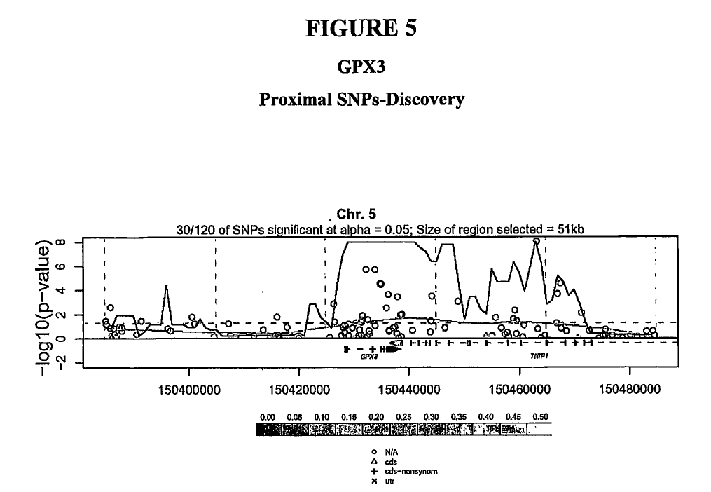

Allelotyping results were considered particularly significant with a calculated p-value of less than or equal to 0.05 for allelotype results. These values are indicated in bold. The allelotyping p-values were plotted in FIG. 4. The position of each SNP on the chromosome is presented on the x-axis. The y-axis gives the negative logarithm (base 10) of the p-value comparing the estimated allele in the case group to that of the control group. The minor allele frequency of the control group for each SNP designated by an X or other symbol on the graphs in FIG. 4 can be determined by consulting Table 23. For example, the left-most X on the left graph is at position 58309549. By proceeding down the Table from top to bottom and across the graphs from left to right the allele frequency associated with each symbol shown can be determined.

To aid the interpretation, multiple lines have been added to the graph. The broken horizontal lines are drawn at two common significance levels, 0.05 and 0.01. The vertical broken lines are drawn every 20 kb to assist in the interpretation of distances between SNPs. Two other lines are drawn to expose linear trends in the association of SNPs to the disease. The light gray line (or generally bottom-most curve) is a nonlinear smoother through the data points on the graph using a local polynomial regression method (W. S. Cleveland, E. Grosse and W. M. Shyu (1992) Local regression models. Chapter 8 of Statistical Models in S eds J. M. Chambers and T. J. Hastie, Wadsworth & Brooks/Cole.). The black line provides a local test for excess statistical significance to identify regions of association. This was created by use of a 10 kb sliding window with 1 kb step sizes. Within each window, a chi-square goodness of fit test was applied to compare the proportion of SNPs that were significant at a test wise level of 0.01, to the proportion that would be expected by chance alone (0.05 for the methods used here). Resulting p-values that were less than 10⁻⁸ were truncated at that value.

Finally, the exons and introns of the genes in the covered region are plotted below each graph at the appropriate chromosomal positions. The gene boundary is indicated by the broken horizontal line. The exon positions are shown as thick, unbroken bars. An arrow is place at the 3′ end of each gene to show the direction of transcription.

### Example 9

### GPX3 Proximal SNPs

It has been discovered that a polymorphic variation (rs869975) in a gene encoding GPX3 is associated with the occurrence of low BMD (see Examples 1 and 2). Two hundred thirty-three additional allelic variants proximal to rs869975 were identified and subsequently allelotyped in low BMD case and high BMD control sample sets as described in Examples 1 and 2. The polymorphic variants are set forth in Table 24. The chromosome position provided in column four of Table 24 is based on Genome “Build 34” of NCBI's GenBank.

TABLE 24

| | Position in | | | | | |
|---|---|---|---|---|---|---|
| | SEQ ID | | Chromosome | | | |
| dbSNP | NO: 5 | Chromosome | Position | Alleles (A1/A2) | genome_letter | deduced_iupac |
| 1478398 | 231 | 5 | 150385031 | a/g | t | Y |
| 1478397 | 330 | 5 | 150385130 | t/c | a | R |
| 1160114 | 582 | 5 | 150385382 | g/c | g | S |
| 1160113 | 589 | 5 | 150385389 | a/g | c | Y |
| 1382323 | 1060 | 5 | 150385860 | a/g | c | Y |
| 1160112 | 1066 | 5 | 150385866 | g/a | t | Y |
| 7709870 | 1311 | 5 | 150386111 | g/a | a | R |
| 7710643 | 1556 | 5 | 150386356 | g/t | g | K |
| 7730467 | 1655 | 5 | 150386455 | t/c | t | Y |
| 6579829 | 1692 | 5 | 150386492 | a/c | a | M |
| 6579830 | 1802 | 5 | 150386602 | g/a | g | R |
| 6579831 | 2061 | 5 | 150386861 | t/a | a | W |
| 6896232 | 2112 | 5 | 150386912 | t/a | t | W |
| 1351131 | 2153 | 5 | 150386953 | t/c | g | R |
| 1038074 | 2667 | 5 | 150387467 | c/t | g | R |
| 1478396 | 3115 | 5 | 150387915 | t/c | a | R |
| 6880512 | 3186 | 5 | 150387986 | g/a | a | R |
| 4958858 | 5621 | 5 | 150390421 | t/c | t | Y |
| 4958431 | 5735 | 5 | 150390535 | t/g | g | K |
| 4958432 | 5829 | 5 | 150390629 | g/c | g | S |
| 6898463 | 6658 | 5 | 150391458 | a/c | c | M |
| 4958859 | 7901 | 5 | 150392701 | g/c | c | S |
| 4130064 | 11447 | 5 | 150396247 | g/a | g | R |
| 4130065 | 11466 | 5 | 150396266 | a/g | a | R |
| 4133119 | 11984 | 5 | 150396784 | t/c | g | R |
| 4958860 | 15803 | 5 | 150400603 | t/g | t | K |
| 4958861 | 16257 | 5 | 150401057 | t/g | t | K |
| 4437356 | 17604 | 5 | 150402404 | c/a | c | M |
| 4958868 | 19762 | 5 | 150404562 | c/t | t | Y |
| 1478400 | 22367 | 5 | 150407167 | a/g | g | R |
| 6889375 | 22709 | 5 | 150407509 | a/g | g | R |
| 1600159 | 23631 | 5 | 150408431 | g/c | c | S |
| 6875892 | 23686 | 5 | 150408486 | t/a | t | W |
| 4608909 | 25599 | 5 | 150410399 | t/c | t | Y |
| 2345000 | 26973 | 5 | 150411773 | a/c | c | M |
| 4516840 | 28457 | 5 | 150413257 | g/t | t | K |
| 2054440 | 28669 | 5 | 150413469 | a/g | a | R |
| 707141 | 29908 | 5 | 150414708 | c/t | a | R |
| 707142 | 30105 | 5 | 150414905 | a/g | t | Y |
| 841236 | 30711 | 5 | 150415511 | a/g | a | R |
| 707143 | 30851 | 5 | 150415651 | g/a | t | Y |
| 707144 | 31203 | 5 | 150416003 | t/c | g | R |
| 6869405 | 31446 | 5 | 150416246 | a/c | c | M |
| 707145 | 31638 | 5 | 150416438 | c/t | g | R |
| 707146 | 33064 | 5 | 150417864 | c/t | g | R |
| 707148 | 33958 | 5 | 150418758 | c/a | t | K |
| 707150 | 35182 | 5 | 150419982 | a/t | t | W |
| 5872184 | 38332 | 5 | 150423132 | a/ac | a | N |
| 3763015 | 40875 | 5 | 150425675 | g/a | c | Y |
| 2042235 | 41624 | 5 | 150426424 | t/c | t | Y |
| 3763013 | 41671 | 5 | 150426471 | a/g | c | Y |
| 2042236 | 41825 | 5 | 150426625 | g/a | g | R |
| 1946234 | 42920 | 5 | 150427720 | c/a | a | M |
| 1946235 | 42935 | 5 | 150427735 | t/c | t | Y |
| 1946236 | 43001 | 5 | 150427801 | t/a | a | W |
| 8177402 | 43012 | 5 | 150427812 | t/c | c | Y |
| 8177403 | 43203 | 5 | 150428003 | c/t | c | Y |
| 8177404 | 43294 | 5 | 150428094 | t/c | t | Y |
| 8177405 | 43295 | 5 | 150428095 | t/c | c | Y |
| 8177406 | 43344 | 5 | 150428144 | c/t | t | Y |
| 8177407 | 43509 | 5 | 150428309 | t/c | c | Y |
| 8177408 | 43549 | 5 | 150428349 | g/c | c | S |
| 8177409 | 43560 | 5 | 150428360 | t/a | a | W |
| 6888961 | 43578 | 5 | 150428378 | a/t | t | W |
| 8177410 | 43640 | 5 | 150428440 | a/g | g | R |
| 8177411 | 43792 | 5 | 150428592 | g/c | g | S |
| 8177412 | 43797 | 5 | 150428597 | c/t | t | Y |
| 8177413 | 43964 | 5 | 150428764 | c/g | g | S |
| 870407 | 44297 | 5 | 150429097 | c/t | a | R |
| 870406 | 44311 | 5 | 150429111 | c/t | g | R |
| 6873202 | 44588 | 5 | 150429388 | a/g | a | R |
| 8177414 | 44775 | 5 | 150429575 | c/t | c | Y |
| 8177415 | 44921 | 5 | 150429721 | c/t | c | Y |
| 3805435 | 45006 | 5 | 150429806 | g/a | t | Y |
| 8177416 | 45098 | 5 | 150429898 | t/c | c | Y |
| 3792799 | 45185 | 5 | 150429985 | c/g | c | S |
| 3792798 | 45475 | 5 | 150430275 | t/c | g | R |
| 3828599 | 45506 | 5 | 150430306 | t/c | a | R |
| 8177417 | 45543 | 5 | 150430343 | g/c | g | S |
| 3792797 | 45601 | 5 | 150430401 | t/g | a | M |
| 8177418 | 45652 | 5 | 150430452 | t/c | c | Y |
| 8177419 | 45756 | 5 | 150430556 | ag/g | g | N |
| 8177420 | 45826 | 5 | 150430626 | t/c | t | Y |
| 8177421 | 45974 | 5 | 150430774 | c/g | g | S |
| 4958872 | 46044 | 5 | 150430844 | t/c | c | Y |
| 3792796 | 46200 | 5 | 150431000 | c/g | g | S |
| 8177422 | 46218 | 5 | 150431018 | a/g | a | R |
| 8177423 | 46221 | 5 | 150431021 | c/t | c | Y |
| 4958434 | 46280 | 5 | 150431080 | c/t | a | R |
| 8177424 | 46330 | 5 | 150431138 | —/gagtcctgg | gagtcctgg | N |
| 8177425 | 46583 | 5 | 150431383 | t/c | c | Y |
| 8177426 | 46650 | 5 | 150431450 | a/g | a | R |
| 8177427 | 46721 | 5 | 150431521 | a/g | a | R |
| 8177429 | 46808 | 5 | 150431608 | g/c | g | S |
| 6889737 | 47242 | 5 | 150432042 | c/a | c | M |
| 3792795 | 47512 | 5 | 150432312 | g/a | c | Y |
| 8177430 | 47600 | 5 | 150432400 | t/c | c | Y |
| 8177431 | 47706 | 5 | 150432506 | a/g | g | R |
| 4958873 | 47806 | 5 | 150432606 | a/g | a | R |
| 8177432 | 47978 | 5 | 150432778 | t/g | t | K |
| 8177433 | 48021 | 5 | 150432821 | t/c | c | Y |
| 8177434 | 48025 | 5 | 150432825 | g/a | a | R |
| 8177435 | 48093 | 5 | 150432893 | t/g | g | K |
| 3763011 | 48413 | 5 | 150433213 | t/c | g | R |
| 8177436 | 48933 | 5 | 150433733 | c/t | t | Y |
| 8177437 | 49097 | 5 | 150433897 | c/g | g | S |
| 4958874 | 49105 | 5 | 150433905 | t/c | t | Y |
| 8177439 | 49570 | 5 | 150434370 | g/a | g | R |
| 8177440 | 49591 | 5 | 150434391 | c/t | c | Y |
| 8177441 | 49704 | 5 | 150434504 | g/c | g | S |
| 8177442 | 49705 | 5 | 150434505 | a/t | a | W |
| 8177443 | 49798 | 5 | 150434598 | c/t | c | Y |
| 869975 | 50082 | 5 | 150434882 | a/g | g | R |
| 869976 | 50147 | 5 | 150434947 | g/a | a | R |
| 8177444 | 50356 | 5 | 150435156 | a/t | a | W |
| 8177445 | 50725 | 5 | 150435525 | t/c | t | Y |
| 7721469 | 50968 | 5 | 150435768 | c/a | a | M |
| 8177446 | 51029 | 5 | 150435829 | a/c | a | M |
| 7704191 | 51086 | 5 | 150435886 | t/c | t | Y |
| 8177447 | 51166 | 5 | 150435966 | t/c | t | Y |
| 11548 | 51493 | 5 | 150436293 | c/t | c | Y |
| 2230303 | 51539 | 5 | 150436339 | g/t | t | K |
| 7722386 | 51562 | 5 | 150436362 | g/a | a | R |
| 8177448 | 51645 | 5 | 150436445 | g/a | g | R |
| 8177449 | 51649 | 5 | 150436449 | t/c | c | Y |
| 2070593 | 51650 | 5 | 150436450 | t/c | g | R |
| 8177450 | 51656 | 5 | 150436456 | g/a | a | R |
| 8177451 | 51657 | 5 | 150436457 | t/c | c | Y |
| 8177452 | 52009 | 5 | 150436809 | g/a | a | R |
| 8177453 | 52143 | 5 | 150436943 | a/c | c | M |
| 8177454 | 52349 | 5 | 150437149 | g/c | g | S |
| 3763010 | 52421 | 5 | 150437221 | c/t | c | Y |
| 8177455 | 52532 | 5 | 150437332 | a/g | g | R |
| 8177456 | 52682 | 5 | 150437482 | a/g | g | R |
| 736775 | 53058 | 5 | 150437858 | t/c | t | Y |
| 2277940 | 53187 | 5 | 150437987 | t/c | t | Y |
| 8177458 | 53377 | 5 | 150438177 | g/a | g | R |
| 8177834 | 53699 | 5 | 150438499 | g/a | c | Y |
| 3924 | 53845 | 5 | 150438645 | a/g | g | R |
| 2233312 | 53920 | 5 | 150438720 | a/g | c | Y |
| 2233311 | 53929 | 5 | 150438729 | t/g | c | M |
| 2233310 | 55473 | 5 | 150440273 | t/c | g | R |
| 2233309 | 55690 | 5 | 150440490 | a/g | c | Y |
| 4958875 | 55850 | 5 | 150440650 | a/g | g | R |
| 2233308 | 56761 | 5 | 150441561 | c/t | g | R |
| 2233307 | 56840 | 5 | 150441640 | c/g | c | S |
| 2233306 | 57000 | 5 | 150441800 | c/t | g | R |
| 2233305 | 57116 | 5 | 150441916 | t/g | a | M |
| 2233304 | 58419 | 5 | 150443219 | t/g | c | M |
| 2233303 | 58420 | 5 | 150443220 | c/t | g | R |
| 2233302 | 58808 | 5 | 150443608 | g/c | c | S |
| 2287719 | 58906 | 5 | 150443706 | a/g | g | R |
| 2287720 | 59048 | 5 | 150443848 | c/t | c | Y |
| 7727034 | 59187 | 5 | 150443987 | c/g | c | S |
| 7727250 | 59361 | 5 | 150444161 | c/t | c | Y |
| 7709800 | 61218 | 5 | 150446018 | g/a | g | R |
| 3840312 | 61700 | 5 | 150446500 | ag/a | a | N |
| 2287721 | 62290 | 5 | 150447090 | g/a | g | R |
| 6875293 | 62596 | 5 | 150447396 | t/c | c | Y |
| 3805434 | 64049 | 5 | 150448849 | g/c | g | S |
| 2080982 | 66077 | 5 | 150450877 | g/t | t | K |
| 2080983 | 66079 | 5 | 150450879 | g/t | g | K |
| 2287722 | 66086 | 5 | 150450886 | t/c | c | Y |
| 2233301 | 66115 | 5 | 150450915 | t/g | c | M |
| 2233300 | 66150 | 5 | 150450950 | c/g | c | S |
| 4958876 | 66475 | 5 | 150451275 | c/a | a | M |
| 2233299 | 69177 | 5 | 150453977 | a/g | g | R |
| 2233298 | 69210 | 5 | 150454010 | a/g | c | Y |
| 2287723 | 69312 | 5 | 150454112 | t/g | t | K |
| 2161359 | 70244 | 5 | 150455044 | a/g | g | R |
| 7734456 | 70882 | 5 | 150455682 | g/c | g | S |
| 4292439 | 71905 | 5 | 150456705 | t/c | c | Y |
| 4958878 | 72294 | 5 | 150457094 | a/t | a | W |
| 6862024 | 72581 | 5 | 150457381 | a/g | g | R |
| 3834819 | 72786 | 5 | 150457589 | —/ca | ca | N |
| 2233297 | 72950 | 5 | 150457750 | g/a | t | Y |
| 2233296 | 73106 | 5 | 150457906 | g/a | c | Y |
| 2233295 | 73162 | 5 | 150457962 | t/c | g | R |
| 2233294 | 73273 | 5 | 150458073 | g/t | a | M |
| 7713028 | 74131 | 5 | 150458931 | g/a | g | R |
| 7713223 | 74406 | 5 | 150459206 | t/c | c | Y |
| 7713567 | 74665 | 5 | 150459465 | c/t | c | Y |
| 888989 | 74740 | 5 | 150459540 | t/c | c | Y |
| 2233293 | 75382 | 5 | 150460182 | a/g | c | Y |
| 3749657 | 75400 | 5 | 150460200 | g/t | c | M |
| 2233292 | 75460 | 5 | 150460260 | g/a | c | Y |
| 2112635 | 75863 | 5 | 150460663 | c/t | c | Y |
| 871269 | 76098 | 5 | 150460898 | t/c | c | Y |
| 3792794 | 78432 | 5 | 150463232 | a/g | c | Y |
| 6579837 | 78604 | 5 | 150463404 | g/t | g | K |
| 3805433 | 79190 | 5 | 150463990 | c/g | c | S |
| 5872186 | 79870 | 5 | 150464670 | —/a | a | N |
| 2233291 | 79928 | 5 | 150464728 | g/c | c | S |
| 2233290 | 80213 | 5 | 150465013 | g/c | g | S |
| 2233289 | 80227 | 5 | 150465027 | c/t | g | R |
| 4958435 | 81994 | 5 | 150466794 | t/g | g | K |
| 4958880 | 82187 | 5 | 150466987 | c/a | c | M |
| 1422673 | 82698 | 5 | 150467498 | t/c | c | Y |
| 2042234 | 82841 | 5 | 150467641 | a/g | a | R |
| 3805432 | 83214 | 5 | 150468014 | a/g | c | Y |
| 3805431 | 83249 | 5 | 150468049 | t/c | g | R |
| 2233288 | 83485 | 5 | 150468285 | t/c | g | R |
| 2233287 | 83807 | 5 | 150468607 | c/t | g | R |
| 3815720 | 83907 | 5 | 150468707 | g/a | g | R |
| 3792792 | 84216 | 5 | 150469016 | a/g | t | Y |
| 3792791 | 84656 | 5 | 150469456 | a/g | c | Y |
| 2303018 | 85448 | 5 | 150470248 | g/a | g | R |
| 3792790 | 85881 | 5 | 150470681 | g/t | a | M |
| 4958436 | 86539 | 5 | 150471339 | t/c | t | Y |
| 2233286 | 86796 | 5 | 150471596 | t/c | g | R |
| 2233285 | 87057 | 5 | 150471857 | g/a | c | Y |
| 7732451 | 87922 | 5 | 150472722 | t/c | a | R |
| 2233284 | 88098 | 5 | 150472898 | t/c | g | R |
| 1422674 | 89319 | 5 | 150474119 | g/t | t | K |
| 3792789 | 89678 | 5 | 150474478 | c/t | g | R |
| 4562032 | 90026 | 5 | 150474826 | c/a | c | M |
| 6865077 | 90033 | 5 | 150474833 | g/a | g | R |
| 1559126 | 90114 | 5 | 150474914 | c/g | c | S |
| 3792788 | 90326 | 5 | 150475126 | t/c | g | R |
| 1559127 | 90463 | 5 | 150475263 | t/c | t | Y |
| 3792786 | 90548 | 5 | 150475348 | a/g | t | Y |
| 6880110 | 90800 | 5 | 150475600 | a/g | a | R |
| 6861227 | 90838 | 5 | 150475638 | g/t | t | K |
| 3805430 | 91400 | 5 | 150476200 | c/g | c | S |
| 1862364 | 92086 | 5 | 150476886 | a/g | a | R |
| 4958881 | 93946 | 5 | 150478746 | t/c | t | Y |
| 3792785 | 95360 | 5 | 150480160 | a/g | t | Y |
| 6869605 | 96576 | 5 | 150481376 | a/c | a | M |
| 6870205 | 96721 | 5 | 150481521 | a/g | g | R |
| 4246047 | 98316 | 5 | 150483116 | t/a | t | W |
| 4958882 | 98497 | 5 | 150483297 | c/g | c | S |
| 3792784 | 99382 | 5 | 150484182 | c/t | a | R |
| 3792783 | 99442 | 5 | 150484242 | t/c | a | R |
| 5872188 | 99764 | 5 | 150484565 | —/ag | ag | N |

Assay for Verifying and Allelotyping SNPs

The methods used to verify and allelotype proximal SNPs of Table 24 are the same methods described in Examples 1 and 2 herein. The primers and probes used in these assays are provided in Table 25 and Table 26, respectively.

TABLE 25

| | | |
|---|---|---|
| DbSNP | | |
| rs# | Forward PCR primer | Reverse PCR primer |

| | | |
|---|---|---|
| 1478398 | TTCCCTCCCTTCCTTTCTTC | AAGATTACCCCACTGCACTC |
| 1478397 | CACATACTTGTCTGAGGATC | AGCACTCAGAATACTACCTG |
| 1160114 | AGCTGGATTCAGAGCCTTTG | TCTGCTATCACCTTGTTCCC |
| 1160113 | TCTGCTATCACCTTGTTCCC | AGCTGGATTCAGAGCCTTTG |
| 1382323 | AGAAGGACAGTTGCAACAGG | GTTCCTCATCCAGTCCTTAC |
| 1160112 | GTTCCTCATCCAGTCCTTAC | AGAAGGACAGTTGCAACAGG |
| 7709870 | TTATGACTCATAACTTTTTG | CTTTCTTGAAATGCTCAGGG |
| 7710643 | ATAAAGTCTGGGCTCAGATG | ATAGACCAGGGAACTGAGAC |
| 7730467 | TGTGGCATATCTTCTATTGC | AATGTGGCTGTTATGGTAGG |
| 6579829 | GGAGGCTTTTTCACATTCAC | AATGTGGCTGTTATGGTAGG |
| 6579830 | TGAGTATGGATTCTCAGGTG | ACCATAAGAGAGAAAAGCCC |
| 6579831 | GTGTGTGATGAATATGGGAG | TTTCTCTCCTGTGTGTATTC |
| 6896232 | GTCACACCTCTTTGTACATC | CACTGAAAGCTCTCTCACAC |
| 1351131 | AAGTGTGAGAGAGCTTTCAG | GTATAAGGTTTCTCTCTAGTG |
| 1038074 | TCATATCTTGTGGGTTGTCC | TTCACTGTGGACCACAAAGG |
| 1478396 | CATGGCCTCTTACAAGACTC | GGGTAATTGTACCCACCAAG |
| 6880512 | GAGGTATTGGTGATTAGGGC | TAAAAAGGAATTCTTGGTGG |
| 4958858 | GCAAGTGAGATCTTGGTAGG | CAGACTGGCAGGAGTACATT |
| 4958431 | CAGAGGTGTCACTTCAGGAA | CCATCTTGCTTCTTTCCCTG |
| 4958432 | TGCCTTAATTCATGCAACCC | TTATATCCAGGGAAAGAAGC |
| 6898463 | CAAATCTACTTTTGAGTACC | GACTCATCAAATGGAGAATG |
| 4958859 | GTGGTATAACTCCTCAGAAG | ATGATGAAGTTGTCTTCAGG |
| 4130064 | AATTCCTCACATTAACCCAG | CCACAGCAAAGACAAAAGGG |
| 4130065 | CCACAGCAAAGACAAAAGGG | TCCTCACATTAACCCAGGAG |
| 4133119 | AGTGAGGTATAATGCCAGCC | AAAATCTCCCTGTGTCTGCC |
| 4958860 | TATGCGGTGGAGAAGAAAGG | TCAACCCGGACAACAAAACC |
| 4958861 | ACTGGGATTTTCTGGTGAAG | ATGGGTGAGTCTCCCTTTAC |
| 4437356 | AGGTTTCTGGGTTTTCTCTC | ATGGTAGTGTGTTTGTTTGC |
| 4958868 | TTGTTGGCCTTGTTCATGTC | CCTGTAATCCCTGCACTTTG |
| 1478400 | TGGACTCCTGTGGGATAATG | CAAGACTTCCAAGTGCATCC |
| 6889375 | TACCTGTCCACAGAAATAGC | CCTGAGACCCTATGACTTTA |
| 1600159 | GGTTCATGTGATTTGGAAGC | TGACTCTCCTAGTTCTTCAG |
| 6875892 | GGGACTTCTCAGCTTCCAAA | AGGGTTCTCCAGAGAAACAG |
| 4608909 | CTGATAGAGGTTTATCTCAG | TCTAGCCAAACTCCTATGTG |
| | C | |
| 2345000 | TAACAAGCCCCTAACTAGTC | TCAGGTGAAACCAACTGAAG |
| 4516840 | GTGCAGTGGATCCTTTTTCC | CTAGACTCTAGGTAGGACAC |
| 2054440 | CCCTTGAATGAGATGGAGAC | TGGGAAAGGAAGGGAATGTC |
| 707141 | ACAGTGCTAAGCACTTTCCC | ACAGTAGACACACAGGATAA |
| 707142 | AGCCTGGCTTCTTCGTCTAC | ATTTCCACCCTGGGCACTTC |
| 841236 | ATGTGTGCATGAGAGGAGAG | CCAAAAAGGAGGAATGTGGG |
| 707143 | CTTTTAAAGCCAGATGGACC | CCACCCTGTTCTTACTGTAG |
| 707144 | ACTGCACCAGACGCTTTCAC | TTGGGCAAGGCGTGTTTCCT |
| 6869405 | TCTCTCATCATGGCTTGTGG | CTGGCCAAGCGATAACACTA |
| 707145 | TAAAGTCTGGGGAGGTCAGG | AGTGGCTGCTTTCGGAACAG |
| 707146 | GCAACAAGAGCGAAACTCTG | AAATGGGCAACATGGCAAAG |
| 707148 | ACCTTTCATATACAGCTGGG | CTGTGTTCCAGCATCATCTG |
| 707150 | ACTGATCTGGGCTAGGAATC | AAGGACAGCCAATTAACACG |
| 5872184 | TAAAATGTTGTTTACCAGGG | AAGGATGCCTATGTGACCAG |
| 3763015 | GCCAAATATTGTTTTCCTTG | ACATCGACCCTTTGTTGTAC |
| 2042235 | TCCTTCTCTGATCTCAGTTG | TTAGGAATGAAAAGCCACAG |
| 3763013 | AGAGACCTGAGATGCTACCC | ACAGGTGGACAACTGAGATC |
| 2042236 | AAAGGTGTTTCCAGCAGATA | AAAATGTAGAGAGATGCCCG |
| 1946234 | AACAAGTCATCTAGCCAGAG | CTGGCTTAGAAGCAGAAAAT |
| 1946235 | CCTGGCTTAGAAGCAGAAAA | GGCACTGGAGATCACTAATC |
| 1946236 | GCACTGGAGATCACTAATC | CAAACCACCTGGCTTAGAAG |
| 8177402 | TATCTGTGGCCAAACCACCT | CACAGGGAGCAAATTTATT |
| 8177403 | TTTCCTTGAGTCTTTGGGTC | GCCTGTAGAAAAAGGTTAGC |
| 8177404 | ATTAGGACTGTAGGGACAGA | GCTAACCTTTTTCTACAGGC |
| 8177405 | ATTAGGACTGTAGGGACAGA | TTTTCTACAGGCATGTCAGC |
| 8177406 | ATGTCAGCTGTGATCCTTAT | GAAAACCCCATTCTGGGTAG |
| 8177407 | TGATGGAAGTCTCCATTCTG | AATTTCCAGAGGACCATCGC |
| 8177408 | GGGCTAATAGCTCCCTAGAA | CGCCAGGTGTTTTTAAAACC |
| 8177409 | ATGCTTCCCAGAATGGAGAC | TCCATTACAGCCAGGGCAAG |
| 6888961 | TCCTCTTTTGGCTCCAAAGT | TTCCATCAGTTCTAGGGAGC |
| 8177410 | TCCTTTCGCACTTTGGAGCC | ATAAGCAAATCTGGGCAGAG |
| 8177411 | TGTGACCAATCCGCGGCCAA | TGAAATCCCAGCCGCCTAGC |
| 8177412 | TGAAATCCCAGCCGCCTAG | TTTCAAGCCCTCGGGTGTGAC |
| 8177413 | TGCAGGCGTCCTGCCTGCT | TCACTCACCTTCGACTTCTC |
| 870407 | GTGACATAGATGTAGCAAGG | AGAATGACTAAGGGAGGAAG |
| 870406 | CGGAGGTGACATAGATGTAG | AGAATGACTAAGGGAGGAAG |
| 6873202 | TAGAAATGGCCACTTGGATT | AGTGTTCTGCACTGTCACTC |
| 8177414 | GGGATGCTTGGAGAAGCTGA | AATGAGGACAGTGGGTCCTG |
| 8177415 | CCTCAGTCTATTTCCAGCAG | ATAAGCATGCTGGGTCATCC |
| 3805435 | GACAGTATGTACTGGGGTGG | TTTGAGTCTATCCCTACACC |
| 8177416 | GAAGACCCAGAGGATTTTAG | AGGGATAGACTCAAAGCGAG |
| 3792799 | GGACAGAATTTTGGAAACGG | CTAAAATCCTCTGGGTCTTC |
| 3792798 | ATTTTCCAGGTCAGGAATGG | CCAAAACTGAAGGTTGGGAC |
| 3828599 | AGTCAGTCCCAACCTTCAGT | CAGGGCCCAATTGTATCTTC |
| 8177417 | AGTTATTCATGGAGTCCACG | TTTTGGAAGGGTCAAAGAAG |
| 3792797 | GAGTCCATTTATCCTTCCCC | ACGTGGACTCCATGAATAAC |
| 8177418 | AAGTCCTGACTGGTTGTATG | CTCAGAGAGAATAGGATGGG |
| 8177419 | ATGCCCATCTCTGCTCCCAA | TCCTAACCCAGAGCTAAGAC |
| 8177420 | ATTCCATTCTCCACATGCCC | TAAGAGTTCAGATGTCTTAG |
| 8177421 | ATTGAAAGGGAGATGCTAGG | TTGTAGAGACAAGGAGCGTG |
| 4958872 | AGTCACCTAGAAACCAGCAG | ACAAAAGGACTGCAGAAGCC |
| 3792796 | CAATATGATGCTGTGAGAAG | ATCTTACAATGGAGGAGCTG |
| 8177422 | ATCTTACAATGGAGGAGCTG | TATGATGCTGTGAGAAGTGG |
| 8177423 | ATCTTACAATGGAGGAGCTG | TATGATGCTGTGAGAAGTGG |
| 4958434 | CTGCTGGTCTTGTGTCTTTC | AGACCACTTCTCACAGCATC |
| 8177424 | GGCTTGAGAGTTGGATGATG | GGAGAGGAGAAAGACACAAG |
| 8177425 | AGAGCAAAACAGGCCTGGAG | ACCCATGGGCCACCTTCTGA |
| 8177426 | ATTTCGTTCCAAGAGACTCC | ATATGTCCAGGGAGATAAGC |
| 8177427 | TTATCTCCCTGGACATATGC | GGCAAGGAGAACCCTATTTC |
| 8177429 | CAGGTTGTCCTAATAATGGG | ATGAAATAGGGTTCTCCTTG |
| 6889737 | CATGACAGAGGCAGCTCTTC | TGAAGGGAGTGATATATGCT |
| 3792795 | GGCCACAAAGGACCTTAAAG | TGACACAATTTGGCAGGTGG |
| 8177430 | AGTAGGATGTGGCCTATGAG | ATTGTGTCAATGATGCAATG |
| 8177431 | CATGATCCTATCAAGCCATC | GACCTGAGTTTCCTCATTTA |
| 4958873 | GCCAGTCCAAGACCACACAG | TGGGAGACCTGGGCTCCAT |
| 8177432 | CCCGGAACATTAACACATGC | TTACCAAATAGGGAAGCAGG |
| 8177433 | GGACAGGAAAGATGAAGCTG | ATGCAGGTACTGTTGCTTTT |
| 8177434 | GGACAGGAAAGATGAAGCTG | ATGCAGGTACTGTTGCTTTT |
| 8177435 | ATTGGGAGCCTCTGAATCAC | GGCCAGTGTGAAGGAAAAAC |
| 3763011 | GTGCTTTTTAAGCACTTAAT | CTCCTAATTCCTTGTTTAAG |
| 8177436 | CACTGTGTTCCGTGGTTTTG | CCTAGAACATGTCCAAAGGC |
| 8177437 | GAGATGGTAACTGAAGCTCC | TGCCAAGAGAAGCACCAAAG |
| 4958874 | TGCCAAGAGAAGCACCAAAG | GAGATGGTAACTGAAGCTCC |
| 8177439 | AGAGAAGCATCCCAGAAAAA | TCCATGCTCTGCCTGGCAAT |
| 8177440 | GCAATCACAGGCGAATTCCC | GCATCCCAGAAAAAGCACTC |
| 8177441 | TCCCCAGTTTCCTCAGTGTC | AACAGAGCAGAGTGCGTTTG |
| 8177442 | TCCCCAGTTTCCTCAGTGTC | AACAGAGCAGAGTGCGTTTG |
| 8177443 | TGCCAAAGATTATGGGAGGG | GAGCAAGAACTATCCATCCC |
| 869975 | CTTTCTCGGGCCTCAGTAGT | CTGTCTCATCCACACCACTC |
| 869976 | AGTGCATTCAGTTCTGGGCC | AGTGGTGTGGATGAGACAGG |
| 8177444 | AGGAGAGTAGTTCTTGGTGG | AGAAAGCTCCTCTCACATGG |
| 8177445 | TTCTCTCCATTGACATCCCC | TCAACAGGTATGTCCGACCA |
| 7721469 | ACTATTGTTCCAACTAGAGG | ATGTTCACTCTGGGTCCTTC |
| 8177446 | AGTTTGGGAGTTAGAAGACT | CAGAAGGACCCAGAGTGAAC |
| 7704191 | TTCCTGGGGAATAGCCTGGC | CGCTGGCAGTCTTCTAACTC |
| | C | |
| 8177447 | TGGGAGGACAGGAGTTCTAG | GCCTCAAGCAAGGTTGACAC |
| 11548 | TGTTCAGGAAGAAATCCGTG | GGGGCCTTGAGTGATAGGA |
| | T | |
| 2230303 | TGCCTGGCAGTACACAGAAC | ACTATCTACCCATCACAGAC |
| 7722386 | CCTGTAGGCATGTGTGTAAA | GCACAAATGGATGCATACAG |
| 8177448 | CAGATGGTACACATTCCCAG | GCCTACAGGTATGCGTGATT |
| 8177449 | CAGATGGTACACATTCCCAG | GCCTACAGGTATGCGTGATT |
| 2070593 | ACACATGCCTACAGGTATGC | CAGATGGTACACATTCCCAG |
| 8177450 | CAGGCACACAGATGGTACAC | GCCTACAGGTATGCGTGATT |
| 8177451 | CAGGCACACAGATGGTACAC | GCCTACAGGTATGCGTGATT |
| 8177452 | TGGCCCTTGCTGTCACATCT | TACATCCCCACCCCACAGTT |
| 8177453 | AGGGGTGGCATCCCTGCCCA | TGAGGGGCCAGCCCTTAGTG |
| 8177454 | TGCACCCACTGTAAAAAGT | AAGTGAGGAGGAGAAGGTGG |
| 3763010 | AGTGGGTGCAAAGGTAATTG | TGATCTCAGGGAGAATTGTG |
| 8177455 | ATGTAGCCCTATTAGGGGTG | AGCTAACTATCAGGTTGTTG |
| 8177456 | CCCGGGAGTTTGAGGCTATA | AGTGCAGTGGCTCTTCACAG |
| 736775 | TACTCTTGAGTAATGGTGAC | TGGCCTAGGCTCCTTCCACT |
| 2277940 | CGAATCATGGACATAAATCC | TCAGGGAAAGGAAGAAAAGG |
| 8177458 | ACAGTAGCCTTGCTGAAGCC | CAGCTTCAGGCTGGCGCCC |
| 8177834 | TCTTAGGATTGCTGCTCCTG | GAAAAACTCCTTCCCTGCTG |
| 3924 | TCCCCAGTCCTGTAAACAGC | GAGCCTCAGCTGGATGAGAG |
| 2233312 | ATTGTGTCATTTGGCTCCAC | CACACCGTGCAAGTGGCTTC |
| 2233311 | TTGTGTCATTTGGCTCCACC | CGTGCAAGTGGCTTCTAGTT |
| 2233310 | CACTGTTTTGGAAAGCTGGG | TTATCTGCCAGCATTGCCTG |
| 2233309 | ATTTAGTATTGTCCTGGCAC | TATTCCGGCTGCGACAAAAC |
| 4958875 | GGGATAACTTGCTGAAGGTC | GCTTAAGAGATTGGCTCTGG |
| 2233308 | AGACACCTTCCAGAGAGGA | AGAGCCAGGCCCTGGACAC |
| 2233307 | TCTTCCATCTGGTGAGTCTG | TCCTCTCTGGAAGGTGTCTG |
| 2233306 | CCACCCAGAACATCTCTGC | ATGGTGTGGCACCATGGCTG |
| 2233305 | CTAGCCTTCAGAGAGCTAAC | TCTGGCTTTGGCCTGCAACA |
| 2233304 | TGGCCCAGAGGGAAGCATCA | CCTGGATCCAGGGCCTAAC |
| 2233303 | TGGCCCAGAGGGAAGCATCA | CCTGGATCCAGGGCCTAAC |
| 2233302 | ACCAGGGCCCTCCAATCCAT | AAATGCCCAGGTAAGAGTGA |
| 2287719 | CTGCAGCTTCTCCACTTGCT | TCGTGAGCGCATGAATGAGG |
| 2287720 | AGGGAGGGTGAAGAGAGGGA | ATCTGTGACCCCAGCAGGAG |
| 7727034 | AGAGTAGTTCTGTGTCCCTC | TGGTTGATGGCTTTCAACGT |
| 7727250 | TTGGATCTGCTGGTTCTGAG | ATGTGAAACTACAGGCTAAG |
| 7709800 | TTGCTCATGACCTAGGCATC | CTTTATCTTCCTACTCCACC |
| 3840312 | GAAATGTGCTGGAAAGCAGG | CCTCTCCAGTTCAGTTTTGC |
| 2287721 | CCTTGAGTCCAGCTGCAATG | GGGTTACCCTTCCTAAGCTG |
| 6875293 | ATGGACATTACTGTACCGAC | TGGCCCAGCCCTTACGTTCT |
| 3805434 | AAATAAGGCCCCTGGTGTCC | GGGCATGGTCTTTTGCCTAG |
| 2080982 | CTGCCTCTTTAGCAGCTTTG | AACTTCTCCTCTGGGCCATA |
| 2080983 | CTGCCTCTTTAGCAGCTTTG | AACTTCTCCTCTGGGCCATA |
| 2287722 | CTGCCTCTTTAGCAGCTTTG | AACTTCTCCTCTGGGCCATA |
| 2233301 | CACAGGGCATGCCTGGTCTT | CCACTCCCCAAGGTTCAAAG |
| 2233300 | TGAACAAGCAGTGGGACCAG | AAGACCAGGCATGCCCTGTG |
| 4958876 | TTTTACCTTGCCAGGAAAGC | CAAAGAGGTGTGTGTGTTCC |
| 2233299 | CCAGCATCTTCACCTTCTTC | TTTTGGTGAAGGCTCCTCTG |
| 2233298 | TTTTGGTGAAGGCTCCTCTG | AGCATCTTCACCTTCTTCTC |
| 2287723 | AATTATCTCACCCAGTCTCG | TGGGAGGAATAGGGAGACAG |
| 2161359 | ACAGCTCTTCCTTACAAAAG | TTAGTGACCATTGGTGGATC |
| 7734456 | AATAGTCTAGAGAGTTCCCC | CCTGGGTTCTATAAGATGTTG |
| 4292439 | CCCTGGACAGGTCTCTTTAC | AGGAAGAGCATCTGGCTCGG |
| 4958878 | AGAGTTTACCCAGGACATGC | TAGAATGGAATACTAGTCCC |
| 6862024 | GTGCTGGGACTAGAGGATAG | TGTTGGCTAGTGTCTGTGTG |
| 3834819 | TGGGAACCACTCTCTAAGAC | GTGTTGAGTCGTTTGCTCAG |
| 2233297 | ATGAAGCCACAGTGACTACC | GTGGTAGAAGGTTCTAGCTG |
| 2233296 | AACCAAGGATGCTACAGAAG | ATGGAAGGGACAGGCAAGAA |
| 2233295 | TTGCCTGTCCCTTCCATCTT | ATCCCAGGGAGGAAAATTTG |
| 2233294 | TGGGATGGAGGTAAACAGAG | CAACTCATGCGATAGTGAGC |
| 7713028 | CTGGTGAAATACATCACCAC | TTGAGTAGCTGGGACTACAG |
| 7713223 | CTTTTGGATGGCCAAGGATG | CCAGCTTCAGTTTCTGAATC |
| 7713567 | AGCTGGTACCCACCAGATGA | ATCCTGTGAGTGGCCACAATT |
| 888989 | CAGGGACTTGATTGGAGTCT | ACTCACAGGATGGAGAGCAA |
| 2233293 | TCAGAGAAGGGGTGAGGAGT | AACCCAGAAATCAGTGCTGC |
| 3749657 | AGAAATCAGTGCTGCACACC | TAAGTTCAGGCCCCTCAGAGA |
| 2233292 | TGAACTTACCGCAGCCTCTC | CCAAGTTGGATAAGGGCCTG |
| 2112635 | TCCATCCCTTCTCCTTCAT | GGACTTCTCTGAGGAGATGG |
| 871269 | TTCCACAAACTTTGAGCTGG | TAATTAATTCCTGAGCTCCC |
| 3792794 | TCTGCTAGAAGGTAAGCTTG | AGTGTGTATGAAGTGCCACG |
| 6579837 | ATAGATCCTAGGCCCCTTAA | AGGAAATAAACAAGGAGGGG |
| | A | |
| 3805433 | CTTAGCCTTGTGGGCTTTAG | AAGCCTCCGTTTTGCCATCT |
| 5872186 | TCCCGTATTTCCCCATTTCC | GCTTTTTGTTCAGGGTGATG |
| 2233291 | CCTTATCCTGTTCATCACCC | AATCCACTGGTACCATGGGG |
| 2233290 | CTGCAGGTGCAGCATCAGG | TACTCAGGCCTTGGTGAAGA |
| 2233289 | TCAGGCCTTGGTGAAGAGTG | ATCAGGTTGCCGTCCTCAC |
| 4958435 | AAGTTCTGCTCATCCTTCCC | GATCTCTGAGGCTCCCTGTA |
| 4958880 | TCTGGAGGAATGGCAATGAC | ATTTCCTCTTGTTAGAATGC |
| 1422673 | ATGCTTGTGTTCACTGGTTG | CATGCTAGTTAGCCAGACAG |
| 2042234 | GCACCTATTGCTCGCATAAC | TCTACCTTCTCCATTCTCCC |
| 3805432 | AAGGCCCAGGGCCCTGTAAG | AACCTCCTTCCTGTGTGCTCC |
| | A | |
| 3805431 | AAATCACCTGTCTTACAGGG | TTTGGGTCTGTGGGCTTCCA |
| 2233288 | ATATGTGAGCGAGAAGCACC | ACAGAACCTGTTCAGATCCC |
| 2233287 | ATGACAGGCTTGTCGAGAAT | TTTTGAGTACACAGGGACCG |
| 3815720 | TGCTGGTTCAAGGTCAGCTA | TAATGTGAGGTCATATCCCC |
| 3792792 | TTCTCAGATCAGTTCACTCC | AACACAGATGGCAGCTGTTAC |
| 3792791 | GATTGCATAGTTCAGCATCC | CATTACCCGTGTAACCTCAG |
| 2303018 | CAAATGTCACAGCATCTCCC | TAAAACTTACACTGGATGGA |
| 3792790 | CATAAAAGCCCCATATCCCC | AGGGAGGGCCAAGTCTCAGT |
| 4958436 | GCCTTGGGGTCTTTATCAGC | ATATGCCAGCTCCAAGTGAC |
| 2233286 | ATGAATGGAAGGGTAGCCTG | ATCCTTCACAGTGAACTCCC |
| 2233285 | TTCCAAAAGCTCCCCTAGAG | ACGGGGAGAAGCAGCACAC |
| 7732451 | CATCTCTCGTGCCCCAGAAA | GCTGGAAGGCCCAGTTAATG |
| 2233284 | TACATGGTGCATAGAGACAG | ATATGCCTCTGCCCAAGTCA |
| 1422674 | TGCTGATAATTCCTCAGCAC | GTCCATTCACTCATCCATTC |
| 3792789 | GAGGATTCCTGATGAAACAG | TTCACTGGGGACCTCAGAAG |
| 4562032 | ATTTTACAAGGGAGAAACTG | CTGACTTTGCATCTTGGCTC |
| 6865077 | CTGACTTTGCATCTTGGCTC | ATTTTACAAGGGAGAAACTG |
| 1559126 | CACCATACTCACACAGCCAG | ATGGTGCTGCCCTACAAAGA |
| 3792788 | CCCTTCTCCCAAAGACTGAC | AGTGTGGGTAGCTGTCCTTT |
| 1559127 | ACTGTTTCTTAGTTCGGTGG | GGTGACTTGTTTTTAACCTG |
| 3792786 | TGAGGACAGAGGCAAGCAGA | AACAGTGTGGGGGCTGCACTT |
| 6880110 | TCAAGAACAGTGAAACAGAG | TGGTGGACAAGAACCAGGAC |
| 6861227 | CATGCCTTCATGTGGTGTTG | AGCAGTGTCCTGGTTCTTGT |
| 3805430 | TCCAGAGAGGGTTTTCCCAC | GGGAGAAAGAAAGGCAGTGG |
| 1862364 | AATCCCATGGCACACCACAG | TGTAGTTTGTGGAACCAGGC |
| 4958881 | GGACTGGAGGTCATCATAAC | TTCCACCCAAGGATGAAAGG |
| 3792785 | TTTGTTCTACCACTAACCAG | CTACATGGATGAGGAAGCAG |
| 6869605 | TTTGTCCAGCTTCTGTGTAC | AGGTGTGAGTTACCTAAGGG |
| 6870205 | GAATCATCAAGAAGGGACTG | ACCCCATTCTTTTTGATGGC |
| 4246047 | AAGCTGGTCTCAAACTCCTG | TGTTATTTAGGCTGAGCGTG |
| 4958882 | TTGTTGGTGGCTGATGAAGG | GCTCACTCATCTGGTTTAAG |
| 3792784 | ATGATTTTCCCATGACTGGC | AAACAGCCAGCTGCTGCTCA |
| 3792783 | AATCTGAAGAGTGACTGTCC | AAAGACCTTGGCGGCCACCAT |
| 5872188 | CCTTTGCCGACTTTGGTTTC | AGACTTGGAAGAGAATGCCC |

TABLE 26

| | | |
|---|---|---|
| dbSNP | Extend | Term |
| rs# | Primer | Mix |

| | | |
|---|---|---|
| 1478398 | TGAGACAGGGCCTCACTCT | ACT |
| 1478397 | GTCTGAGGATCAGTAATAATAC | ACT |
| 1160114 | CTTAATTGCAATGCCTCTT | ACT |
| 1160113 | CTTGTTCCCTACCTAGC | ACT |
| 1382323 | CTGACCAGGGTAACAAC | ACT |
| 1160112 | TCTCCAATCACCGTTGT | ACG |
| 7709870 | CCTACAAGAAATATCAAGGC | ACG |
| 7710643 | GAGCATTACAGTTGTCAC | CGT |
| 7730467 | TTGCCACTAAATAAATCCAA | ACT |
| 6579829 | CATTCACTATGCTCTTTTTTT | ACT |
| 6579830 | ATTCCCTGCTGAAGGTT | ACG |
| 6579831 | AATATGGGAGAGCCTTC | CGT |
| 6896232 | AATACACACAGGAGAGAAA | CGT |
| 1351131 | GAGAGCTTTCAGTGAAGT | ACT |
| 1038074 | GTGGGTTGTCCACATAT | ACG |
| 1478396 | CAAGACTCACTGCAATTTA | ACT |
| 6880512 | GGGCTTCAACATATGAAT | ACG |
| 4958858 | GAAGATGAAGGCCTGAG | ACT |
| 4958431 | CTTCAGGAAAATCCGGC | ACT |
| 4958432 | AAATACAGTCAGCCCCC | ACT |
| 6898463 | CTTCATTTCAGGTATTCTACT | ACT |
| 4958859 | TCAGAAGAGGAGAGCAC | ACT |
| 4130064 | CCTTCCAAAATTTCCCT | ACG |
| 4130065 | GGGAAATTTTGGAAGGT | ACT |
| 4133119 | GCCAAGTATCCTGTATCAA | ACT |
| 4958860 | AGGGCAACAATAAGGGA | ACT |
| 4958861 | AAGGAGCAGAAGCATGA | ACT |
| 4437356 | CTGAGCAGCCCTAGTGA | CGT |
| 4958868 | CTCCTGACCTCGTGATC | ACG |
| 1478400 | TGGGTGAAAAAATTGGTA | ACT |
| 6889375 | CTGCTGATGATGTAGGAAT | ACT |
| 1600159 | GAAGCTGAGAAGTCCCG | ACT |
| 6875892 | CCAATTCATTATAGGAAATTTTTT | CGT |
| 4608909 | GGTTTATCTCAGCATTGTTTAT | ACT |
| 2345000 | CTAGTCTCCTTGTTCTCTTA | ACT |
| 4516840 | TCCTTTTTCCACCTTGC | CGT |
| 2054440 | GTAAACATCCAAAAGCATTTC | ACT |
| 707141 | ACATCCTGAGATGAAGG | ACG |
| 707142 | CAGGCAGCAGCAGCACA | ACT |
| 841236 | ACCTTCTCTGAGGGTATTCTA | ACT |
| 707143 | CCCTTAGAGGTCATCTGGTC | ACG |
| 707144 | CCATACAAAATCCCCTTG | ACT |
| 6869405 | TGACTGGGACCCAGGCT | ACT |
| 707145 | GGCCCAGCAGCCAGAAG | ACG |
| 707146 | GAAAAGAAAGGCTAAGTGA | ACG |
| 707148 | ACTGAGGCCCAGAGAGG | CGT |
| 707150 | GTCTGCGCCACCTTCCC | CGT |
| 5872184 | GGAGACTCAATGCCAGG | ACT |
| 3763015 | CTGTGCTGGTGCTGGTG | ACG |
| 2042235 | ATCTCAGTTGTCCACCT | ACT |
| 3763013 | TACCCCTGGATTGCTAC | ACT |
| 2042236 | AGGCACCTAGGGTCACA | ACG |
| 1946234 | CACTGGAGATCACTAATC | CGT |
| 1946235 | CCTCAGTTTCCTCAGCT | ACT |
| 1946236 | ACACAGGGAGCAAATTT | CGT |
| 8177402 | CCAAACCACCTGGCTTA | ACT |
| 8177403 | GTCTTTGGGTCTTCATTT | ACG |
| 8177404 | TCCTCCCCATCATAAGG | ACT |
| 8177405 | TTCCTCCCCATCATAAG | ACT |
| 8177406 | CTCTGTCCCTACAGTCC | ACG |
| 8177407 | GCATTTCTTATTCCTATCGAG | ACT |
| 8177408 | GCTCCCTAGAACTGATG | ACT |
| 8177409 | GAGACTTCCATCAGTTCT | CGT |
| 6888961 | CCATTACAGCCAGGGCA | CGT |
| 8177410 | CTTTGGAGCCAAAAGAG | ACT |
| 8177411 | GCGGCCAAGCCGAGACC | ACT |
| 8177412 | GATTGGCTGCAAGGGTC | ACG |
| 8177413 | TCCTGCCTGCTTTCCCT | ACT |
| 870407 | GACCCTCGAGGTGGCAG | ACG |
| 870406 | AGATGTAGCAAGGCGAC | ACG |
| 6873202 | CAGAGATCTGTGCTAGAA | ACT |
| 8177414 | TGTTCTCTGAAATTTCCTC | ACG |
| 8177415 | AGCAGGTGTGTGGCCCA | ACG |
| 3805435 | TACTGGGGTGGGCTCTG | ACG |
| 8177416 | TGCATGTTGGGAAGTTG | ACT |
| 3792799 | CGGAATCCTAGACTCATAA | ACT |
| 3792798 | ATAGACAGGCCAGCACC | ACT |
| 3828599 | CAACCTTCAGTTTTGGAA | ACT |
| 8177417 | ACGTTCTCCCCACTAGC | ACT |
| 3792797 | ATCCTTCCCCAGAGTGC | ACT |
| 8177418 | CTCACCCTAAAACTTTTCTA | ACT |
| 8177419 | CTGCTCCCAAGTTCTTA | ACT |
| 8177420 | ACATGCCCCATAACCCT | ACT |
| 8177421 | TCCCCAGTCCAACTTCA | ACT |
| 4858872 | GCAGTTACTAGGATCCC | ACT |
| 3792796 | TTGCTGTGTCTCTGATG | ACT |
| 8177422 | CATCAGAGACACAGCAA | ACT |
| 8177423 | CAGAGACACAGCAAAGC | ACG |
| 4958434 | TCTCCTCTCCCGTCTTAT | ACG |
| 8177424 | TGGATGATGGGACTCAG | ACG |
| 8177425 | GAACGAAATCTCATGTCAA | ACT |
| 8177426 | AGCAGATTCCCACAACC | ACT |
| 8177427 | GACTTGCTCAGGGCCAC | ACT |
| 8177429 | CCTAATAATGGGAACTTGTAAA | ACT |
| 6889737 | GGCAGCTCTTCTGCACT | CGT |
| 3792795 | TTCAAGGTTTCTCCTTTC | ACG |
| 8177430 | ATGAGCCTACTCTGCCC | ACT |
| 8177431 | CATGGTTGACCTGGTTG | ACT |
| 4958873 | ACAGGGAGTTAATGGCA | ACT |
| 8177432 | TGTTGCTTTTCCCTACC | ACT |
| 8177433 | GCTGTTGTTACCAAATAGG | ACT |
| 8177434 | GAAGCTGTTGTTACCAAA | ACG |
| 8177435 | GAATCACCATGTCCATAC | ACT |
| 3763011 | AAGCACTTAATATTAAGTACCC | ACT |
| 8177436 | TGTGAAGATGATTATATAAGCC | ACG |
| 8177437 | AAGCTCCATCTTGCTGA | ACT |
| 4958874 | TGGGGCTGAGGGAAAAT | ACT |
| 8177439 | TTTCACTTCCGGGAATT | ACG |
| 8177440 | TCCCGGAAGTGAAAGGA | ACG |
| 8177441 | TCAGTGTCCCCTGGTCT | ACT |
| 8177442 | CTCAGTGTCCCCTGGTC | CGT |
| 8177443 | GAGGGTTGTAACCTCAC | ACG |
| 869975 | GGGCCTCAGTAGTTCCAGC | ACT |
| 869976 | CCAAAGAGAAAGAGCAGA | ACG |
| 8177444 | GATAAATGTCCACCATGA | CGT |
| 8177445 | CTGGAAATTAGGGACAA | ACT |
| 7721469 | ACCCTGACTAGGGTCTC | CGT |
| 8177446 | GCTGTGGCTCTAGAAGA | ACT |
| 7704191 | ATAGCCTGGCCCTGAGA | ACT |
| 8177447 | AGTTCTAGAGCAGGGAT | ACT |
| 11548 | CACACTATCTACCCATCA | ACG |
| 2230303 | GTACACAGAACTGTATGC | CGT |
| 7722386 | ACCCACACCCACATGCC | ACG |
| 8177448 | CATAGGTAGACACGTGG | ACG |
| 8177449 | AAGACACATAGGTAGACAC | ACT |
| 2070593 | GCATGGGTGTACAGCCAC | ACT |
| 8177450 | CCAGAAAGACACATAGG | ACG |
| 8177451 | CCCAGAAAGACACATAG | ACT |
| 8177452 | TGTCACATCTGCCTTGG | ACG |
| 8177453 | CCTGCCCAGGGGCCTTA | ACT |
| 8177454 | TCTTTTCTCATCCTCCC | ACT |
| 3763010 | AAAGGTAATTGCGGTTTT | ACG |
| 8177455 | GGTGGCATCTTCATGAG | ACT |
| 8177456 | TGAGGCTATAGTGTGCC | ACT |
| 736775 | TCCCTGAGGGTAGGGCA | ACT |
| 2277940 | CATGTTCATTTATGCGCT | ACT |
| 8177458 | GCCATGCCAGCCACGTC | ACG |
| 8177834 | CCTGGAGGCTTCTGCAA | ACG |
| 3924 | CTCAGTTCAGGGACTGGT | ACT |
| 2233312 | TGATCTCAGATTGCCAA | ACT |
| 2233311 | CAGAGCCAGCTGATCTCA | ACT |
| 2233310 | CCAAGAGCAGAAACTAAC | ACT |
| 2233309 | TGCTATTGTAATTTTTGGGT | ACT |
| 4958875 | CTGAAGGTCATCTAGCAA | ACT |
| 2233308 | CTTCTGAACTAAACAGCAC | ACG |
| 2233307 | GCTCCAGGGCACACACA | ACT |
| 2233306 | CTACCCCTACGCCTACC | ACG |
| 2233305 | GATGAGATGGGTGTCCT | ACT |
| 2233304 | TCAGCCGGTCAGTCCTC | ACT |
| 2233303 | ATCAGCCGGTCAGTCCT | ACG |
| 2233302 | TCCATGCCCCCTCTCCC | ACT |
| 2287719 | CTCCACTTGCTTCTTCA | ACT |
| 2287720 | TTCTCCTCAGGCCCAGA | ACG |
| 7727034 | ACATTCTGGGCTTCAAG | ACT |
| 7727250 | AGAGACCCTACAAACTC | ACG |
| 7709800 | CCTAGGCATCTCCCTGT | ACG |
| 3840312 | CTAGAGCCGTTCCCACC | CGT |
| 2287721 | CCCATCACTGGCACCCC | ACG |
| 8875293 | GAGCATTGTGAAGTGATG | ACT |
| 3805434 | TGCTGACGGGAGGAACT | ACT |
| 2080982 | AACCTTGGGGAGTGGCC | CGT |
| 2080983 | TGAACCTTGGGGAGTGG | CGT |
| 2287722 | GCAGCTTTGAACCTTGG | ACT |
| 2233301 | TGCCTGGTCTTCATCTC | ACT |
| 2233300 | GGTAATGTGGGGTTCCT | ACT |
| 4958876 | CTAAGACATGGAAACTACAT | CGT |
| 2233299 | TCACCTTCTTCTCGGCTGC | ACT |
| 2233298 | CCCCAGGCTAGTGTGAC | ACT |
| 2287723 | AAGACCCCTCACCCAAG | ACT |
| 2181359 | CATTCCATTACTACAGTAATAACT | ACT |
| 7734456 | CCCTTCCTTTACTTCCC | ACT |
| 4292439 | CCCTTTAATCTCCACTC | ACT |
| 4958878 | CAGGACATGCAAATCTATT | CGT |
| 6862024 | CTAGAGGATAGGGGAGT | ACT |
| 3834819 | CTTGACAAACTGGAATGT | ACT |
| 2233297 | ATGGAGGTCAGGCCAAG | ACG |
| 2233296 | ACATACCTGCTGCTGTC | ACG |
| 2233295 | AGCCTGGCCGCCCAGAC | ACT |
| 2233294 | CCCTGAGTTAAGAAACCT | CGT |
| 7713028 | TACATCACCACCCCTCC | ACG |
| 7713223 | GGGGTTTTTATAGCCCTA | ACT |
| 7713567 | ACCAGATGACTCATCAC | ACG |
| 888989 | GATTGGAGTCTTACAACA | ACT |
| 2233293 | GGGGCTGCAGGAGGAGG | ACT |
| 3749657 | CCTCCTCCTGCAGCCCC | CGT |
| 2233292 | TCTCGGCAGCCTGATCC | ACG |
| 2112635 | CCCCAGCTGACTTCTAC | ACG |
| 871269 | CTGGCAGTGAACAAGAG | ACT |
| 3792794 | TGTTGTATTCACTGTTGC | ACT |
| 6579837 | AGGGGTCCTGCCAACCT | CGT |
| 3805433 | TGGGCTTTAGCAGCGGG | ACT |
| 5872186 | CCTCATCCCTCCCCACA | CGT |
| 2233291 | GTCTGCCCACAACACAG | ACT |
| 2233290 | GTTGCCGTCCTCACGGG | ACT |
| 2233289 | CCCTTTCCCTCTCCAGG | ACG |
| 4958435 | ATGGGAATCACAGGACA | ACT |
| 4958880 | GAATGGCAATGACTATAACC | CGT |
| 1422673 | TGTCAGTGCCCAGTGCA | ACT |
| 2042234 | CTCGCATAACAACAATTAAAG | ACT |
| 3805432 | CCTGTAAGACAGGTGATTT | ACT |
| 3805431 | GGGCCCTGGGCCTTCCT | ACT |
| 2233288 | CCCCAAGTGACTCCAGG | ACT |
| 2233287 | AATCCTGCTTTGTGATCT | ACG |
| 3815720 | AAGGTCAGCTAGTCCCA | ACG |
| 3792792 | GGGAAGGCCAGCACAGG | ACT |
| 3792791 | AGGTGCAGGGAAAGAAG | ACT |
| 2303018 | CAGCATCTCCCACAGCC | ACG |
| 3792790 | GCTTGGGAAGGTCATCA | CGT |
| 4958436 | TGTGTGCCAGGCATCGC | ACT |
| 2233286 | GATAAACGAGAGAATGTGG | ACT |
| 2233285 | GGAAGCAAAAGCATTTACT | ACG |
| 7732451 | GTGCCCCAGAAAGAGGAG | ACT |
| 2233284 | GTGAGGCAGGAGCCAGC | ACT |
| 1422674 | CACCCAGACTGGTGCCT | CGT |
| 3792789 | CCTTGGGAGGAAGGTGC | ACG |
| 4562032 | TAACTCAGCCACGATGA | CGT |
| 6865077 | GCATCTTGGCTCTTATTG | ACG |
| 1559126 | ACAGCCAGGACACAGAG | ACT |
| 3792788 | CAGATACAAGATGAATACACC | ACT |
| 1559127 | TCGGTGGAAACATCTGC | ACT |
| 3792786 | GACCCCCAATGTCTGCC | ACT |
| 6880110 | GCCACCTTGCATGACAA | ACT |
| 6861227 | GAGTTCCATTTAGGGTG | CGT |
| 3805430 | CAATAGGATATTTCTCCTGC | ACT |
| 1862364 | CCCCACCACATCTTCTC | ACT |
| 4958881 | GCAATGTGATATCATGGC | ACT |
| 3792785 | TCTCAGGCAAATGACTT | ACT |
| 6869605 | CTCTAGATTCCTAGATAGGG | ACT |
| 6870205 | ACAAACCACCATTCATTC | ACT |
| 4246047 | TGCTCTGCCTCCCAAAG | CGT |
| 4958882 | TGATGAAGGAGAAATTTCAA | ACT |
| 3792784 | TCACTCTTCAGATTGGAA | ACG |
| 3792783 | AAATCATCAAGAACTTCCC | ACT |
| 5872188 | GAATGAAAATGTTTCACTCT | ACT |

Genetic Analysis

Allelotyping results are shown for female cases and controls in Table 27. The allele frequency for A2 allele is noted in the fifth and sixth columns for control pools and case pools, respectively, where “AF” is allele frequency. Some SNPs do not have an allele frequency disclosed because of failed assays.

TABLE 27

| | Position in | | | | | | | Low BMD |
|---|---|---|---|---|---|---|---|---|
| dbSNP | SEQ ID | Chromo-some | A1/A2 | Control AF | Case AF | | | Associated |
| rs# | NO: 5 | Position | Allele | (High BMD) | (Low BMD) | p-Value | OR | Allele |
| 1478398 | 231 | 150385031 | A/G | A = 0.28 | A = 0.34 | 0.0334 | 1.31 | A |
| | | | | G = 0.72 | G = 0.66 | | | |
| 1478397 | 330 | 150385130 | T/C | T = 0.61 | T = 0.67 | 0.0621 | 1.29 | T |
| | | | | C = 0.39 | C = 0.33 | | | |
| 1160114 | 582 | 150385382 | G/C | G = 0.96 | G = 0.92 | 0.0034 | 0.44 | C |
| | | | | C = 0.04 | C = 0.08 | | | |
| 1160113 | 589 | 150385389 | A/G | A = 0.96 | A = 0.93 | 0.0169 | 0.51 | G |
| | | | | G = 0.04 | G = 0.07 | | | |
| 1382323 | 1060 | 150385860 | A/G | A = 0.67 | A = 0.63 | 0.1288 | 0.83 | G |
| | | | | G = 0.33 | G = 0.37 | | | |
| 1160112 | 1066 | 150385866 | G/A | G = 0.91 | G = 0.85 | 0.0025 | 0.57 | A |
| | | | | A = 0.09 | A = 0.15 | | | |
| 7709870 | 1311 | 150386111 | G/A | G = 0.11 | G = 0.12 | 0.5796 | 1.11 | G |
| | | | | A = 0.89 | A = 0.88 | | | |
| 7710643 | 1556 | 150386356 | G/T | G = 0.87 | G = 0.86 | 0.7100 | 0.93 | T |
| | | | | T = 0.13 | T = 0.14 | | | |
| 7730467 | 1655 | 150386455 | T/C | T = 1.00 | T = 1.00 | | | |
| | | | | C = 0.00 | C = 0.00 | | | |
| 6579829 | 1692 | 150386492 | A/C | A = 0.92 | A = 0.89 | 0.1423 | 0.74 | C |
| | | | | C = 0.08 | C = 0.11 | | | |
| 6579830 | 1802 | 150386602 | G/A | G = 0.91 | G = 0.89 | 0.4377 | 0.86 | A |
| | | | | A = 0.09 | A = 0.11 | | | |
| 6579831 | 2061 | 150386861 | T/A | T = 0.31 | T = 0.32 | 0.7948 | 1.04 | T |
| | | | | A = 0.69 | A = 0.68 | | | |
| 6896232 | 2112 | 150386912 | T/A | T = 0.90 | T = 0.89 | 0.7880 | 0.94 | A |
| | | | | A = 0.10 | A = 0.11 | | | |
| 1351131 | 2153 | 150386953 | T/C | T = 0.51 | T = 0.49 | 0.4152 | 0.91 | C |
| | | | | C = 0.49 | C = 0.51 | | | |
| 1038074 | 2667 | 150387467 | C/T | C = 0.23 | C = 0.27 | 0.1062 | 1.25 | C |
| | | | | T = 0.77 | T = 0.73 | | | |
| 1478396 | 3115 | 150387915 | T/C | T = 0.92 | T = 0.90 | 0.1123 | 0.71 | C |
| | | | | C = 0.08 | C = 0.10 | | | |
| 6880512 | 3186 | 150387986 | G/A | G = 0.64 | G = 0.60 | 0.2313 | 0.86 | A |
| | | | | A = 0.36 | A = 0.40 | | | |
| 4958858 | 5621 | 150390421 | T/C | T = 0.06 | T = 0.13 | ˜0.00001 | 2.54 | T |
| | | | | C = 0.94 | A = 0.87 | | | |
| 4958431 | 5735 | 150390535 | T/G | T = 0.98 | T = 0.95 | 0.0132 | 0.44 | G |
| | | | | G = 0.02 | G = 0.05 | | | |
| 4958432 | 5829 | 150390629 | G/C | G = 0.86 | G = 0.85 | 0.4357 | 0.87 | C |
| | | | | C = 0.14 | C = 0.15 | | | |
| 6898463 | 6658 | 150391458 | A/C | A = 0.86 | A = 0.82 | 0.0341 | 0.71 | C |
| | | | | C = 0.14 | C = 0.18 | | | |
| 4958859 | 7901 | 150392701 | G/C | G = 0.95 | G = 0.90 | 0.0030 | 0.48 | C |
| | | | | C = 0.05 | C = 0.10 | | | |
| 4130064 | 11447 | 150396247 | G/A | G = 0.26 | G = 0.30 | 0.1514 | 1.21 | G |
| | | | | A = 0.74 | A = 0.70 | | | |
| 4130065 | 11466 | 150396266 | A/G | A = 0.16 | A = | | | |
| | | | | G = 0.84 | G = | | | |
| 4133119 | 11984 | 150396784 | T/C | T = 0.95 | T = 0.94 | 0.2216 | 0.72 | C |
| | | | | C = 0.05 | C = 0.06 | | | |
| 4958860 | 15803 | 150400603 | T/G | T = 0.10 | T = 0.17 | 0.0163 | 1.88 | T |
| | | | | G = 0.90 | G = 0.83 | | | |
| 4958861 | 16257 | 150401057 | T/G | T = 0.13 | T = 0.17 | 0.0581 | 1.38 | T |
| | | | | G = 0.87 | G = 0.83 | | | |
| 4437356 | 17604 | 150402404 | C/A | C = 0.10 | C = | | | |
| | | | | A = 0.90 | A = | | | |
| 4958868 | 19762 | 150404562 | C/T | C = 0.55 | C = 0.53 | 0.6623 | 0.94 | T |
| | | | | T = 0.45 | T = 0.47 | | | |
| 1478400 | 22367 | 150407167 | A/G | A = 0.96 | A =0.94 | 0.0584 | 0.57 | G |
| | | | | G = 0.04 | G = 0.06 | | | |
| 6889375 | 22709 | 150407509 | A/G | A = 0.59 | A = 0.61 | 0.5378 | 1.09 | A |
| | | | | G = 0.41 | G = 0.39 | | | |
| 1600159 | 23631 | 150408431 | G/C | G = 0.62 | G = 0.61 | 0.7340 | 0.96 | C |
| | | | | C = 0.38 | C = 0.39 | | | |
| 6875892 | 23686 | 150408486 | T/A | T = 1.00 | T = 1.00 | | | |
| | | | | A = 0.00 | A = 0.00 | | | |
| 4608909 | 25599 | 150410399 | T/C | T = 0.98 | T = 0.92 | 0.0008 | 0.27 | C |
| | | | | C = 0.02 | C = 0.08 | | | |
| 2345000 | 26973 | 150411773 | A/C | A = 0.57 | A = 0.55 | 0.5708 | 0.93 | C |
| | | | | C = 0.43 | C = 0.45 | | | |
| 4516840 | 28457 | 150413257 | G/T | G = 0.00 | G = 0.00 | | | |
| | | | | T = 1.00 | T = 1.00 | | | |
| 2054440 | 28669 | 150413469 | A/G | A = 0.62 | A = 0.66 | 0.1835 | 1.18 | A |
| | | | | G = 0.38 | G = 0.34 | | | |
| 707141 | 29908 | 150414708 | C/T | C = 0.45 | C = 0.46 | 0.6490 | 1.06 | C |
| | | | | T = 0.55 | T = 0.54 | | | |
| 707142 | 30105 | 150414905 | A/G | A = 0.54 | A = 0.53 | 0.7349 | 0.96 | G |
| | | | | G = 0.46 | G = 0.47 | | | |
| 841236 | 30711 | 150415511 | A/G | A = 0.55 | A = 0.54 | 0.7731 | 0.97 | G |
| | | | | G = 0.45 | G = 0.46 | | | |
| 707143 | 30851 | 150415651 | G/A | G = 0.64 | G = 0.66 | 0.6049 | 1.07 | G |
| | | | | A = 0.36 | A = 0.34 | | | |
| 707144 | 31203 | 150416003 | T/C | T = 0.69 | T = 0.75 | 0.0163 | 1.38 | T |
| | | | | C = 0.31 | C = 0.25 | | | |
| 6869405 | 31446 | 150416246 | A/C | A = 0.08 | A = 0.09 | 0.5734 | 1.13 | A |
| | | | | C = 0.92 | C = 0.91 | | | |
| 707145 | 31638 | 150416438 | C/T | C = 0.62 | C = 0.61 | 0.8223 | 0.97 | T |
| | | | | T = 0.38 | T = 0.39 | | | |
| 707146 | 33064 | 150417864 | C/T | C = 0.53 | C = 0.48 | 0.1214 | 0.83 | T |
| | | | | T = 0.47 | T = 0.52 | | | |
| 707148 | 33958 | 150418758 | C/A | C = 0.46 | C = 0.48 | 0.6249 | 1.06 | C |
| | | | | A = 0.54 | A = 0.52 | | | |
| 707150 | 35182 | 150419982 | A/T | A = 0.67 | A = 0.67 | 0.9445 | 0.99 | A |
| | | | | T = 0.33 | T = 0.33 | | | |
| 5872184 | 38332 | 150423132 | —/C | — = 0.00 | — = 0.00 | | | |
| | | | | C = 1.00 | C = 1.00 | | | |
| 3763015 | 40875 | 150425675 | G/A | G = 0.61 | G = 0.60 | 0.7976 | 0.97 | A |
| | | | | A = 0.39 | A = 0.40 | | | |
| 2042235 | 41624 | 150426424 | T/C | T = 0.13 | T = 0.20 | 0.0014 | 1.70 | T |
| | | | | C = 0.87 | C = 0.80 | | | |
| 3763013 | 41671 | 150426471 | A/G | A = 0.97 | A = 0.82 | ˜0.00001 | 0.14 | G |
| | | | | G = 0.03 | G = 0.18 | | | |
| 2042236 | 41825 | 150426625 | G/A | G = 0.90 | G = 0.86 | 0.0420 | 0.69 | A |
| | | | | A = 0.10 | A = 0.14 | | | |
| 1946234 | 42920 | 150427720 | C/A | C = 0.00 | C = 0.00 | | | |
| | | | | A = 1.00 | A = 1.00 | | | |
| 1946235 | 42935 | 150427735 | T/C | T = | T = 0.87 | | | |
| | | | | C = | C = 0.13 | | | |
| 1946236 | 43001 | 150427801 | T/A | T = 0.19 | T = 0.20 | 0.5482 | 1.10 | T |
| | | | | A = 0.81 | A = 0.80 | | | |
| 8177402 | 43012 | 150427812 | T/C | T = 0.00 | T = 0.00 | | | |
| | | | | C = 1.00 | C = 1.00 | | | |
| 8177403 | 43203 | 150428003 | C/T | C = 1.00 | C = 1.00 | | | |
| | | | | T = 0.00 | T = 0.00 | | | |
| 8177404 | 43294 | 150428094 | T/C | T = 0.97 | T = 0.94 | 0.0363 | 0.52 | C |
| | | | | C = 0.03 | C = 0.06 | | | |
| 8177405 | 43295 | 150428095 | T/C | T = 0.00 | T = 0.00 | | | |
| | | | | C = 1.00 | C = 1.00 | | | |
| 8177406 | 43344 | 150428144 | C/T | C = 0.09 | C = 0.11 | 0.1222 | 1.36 | C |
| | | | | T = 0.91 | T = 0.89 | | | |
| 8177407 | 43509 | 150428309 | T/C | T = 0.00 | T = 0.00 | | | |
| | | | | C = 1.00 | C = 1.00 | | | |
| 8177408 | 43549 | 150428349 | G/C | G = 0.00 | G = 0.00 | | | |
| | | | | C = 1.00 | C = 1.00 | | | |
| 8177409 | 43560 | 150428360 | T/A | T = 0.23 | T = 0.28 | 0.0779 | 1.29 | T |
| | | | | A = 0.77 | A = 0.72 | | | |
| 6888961 | 43578 | 150428378 | A/T | A = 0.08 | A = 0.07 | 0.3176 | 0.79 | T |
| | | | | T = 0.92 | T = 0.93 | | | |
| 8177410 | 43640 | 150428440 | A/G | A = | A = 0.00 | | | |
| | | | | G = | G = 1.00 | | | |
| 8177411 | 43792 | 150428592 | G/C | G = 1.00 | G = 1.00 | | | |
| | | | | C = 0.00 | C = 0.00 | | | |
| 8177412 | 43797 | 150428597 | C/T | C = 0.15 | C = 0.18 | 0.0943 | 1.31 | C |
| | | | | T = 0.85 | T = 0.82 | | | |
| 8177413 | 43964 | 150428764 | C/G | C = | C = 0.01 | | | |
| | | | | G = | G = 0.99 | | | |
| 870407 | 44297 | 150429097 | C/T | C = 0.07 | C = 0.11 | 0.0641 | 1.49 | C |
| | | | | T = 0.93 | T = 0.89 | | | |
| 870406 | 44311 | 150429111 | C/T | C = 0.89 | C = 0.88 | 0.5675 | 0.87 | T |
| | | | | T = 0.11 | T = 0.12 | | | |
| 6873202 | 44588 | 150429388 | A/G | A = 1.00 | A = 1.00 | | | |
| | | | | G = 0.00 | G = 0.00 | | | |
| 8177414 | 44775 | 150429575 | C/T | C = 1.00 | C = 1.00 | | | |
| | | | | T = 0.00 | T = 0.00 | | | |
| 8177415 | 44921 | 150429721 | C/T | C = 1.00 | C = 1.00 | | | |
| | | | | T = 0.00 | T = 0.00 | | | |
| 3805435 | 45006 | 150429806 | G/A | G = 0.10 | G = 0.07 | 0.1318 | 0.71 | A |
| | | | | A = 0.90 | A = 0.93 | | | |
| 8177416 | 45098 | 150429898 | T/C | T = 0.00 | T = 0.00 | | | |
| | | | | C = 1.00 | C = 1.00 | | | |
| 3792799 | 45185 | 150429985 | C/G | C = 0.00 | C = 0.00 | | | |
| | | | | G = 1.00 | G = 1.00 | | | |
| 3792798 | 45475 | 150430275 | T/C | T = 0.05 | T = 0.10 | 0.0906 | 2.06 | T |
| | | | | C = 0.95 | C = 0.90 | | | |
| 3828599 | 45506 | 150430306 | T/C | T = 0.55 | T = 0.55 | 0.9993 | 1.00 | T |
| | | | | C = 0.45 | C = 0.45 | | | |
| 8177417 | 45543 | 150430343 | G/C | G = 1.00 | G = 1.00 | | | |
| | | | | C = 0.00 | C = 0.00 | | | |
| 3792797 | 45601 | 150430401 | T/G | T = 0.46 | T = 0.52 | 0.0520 | 1.27 | T |
| | | | | G = 0.54 | G = 0.48 | | | |
| 8177418 | 45652 | 150430452 | T/C | T = 0.00 | T = 0.00 | | | |
| | | | | C = 1.00 | C = 1.00 | | | |
| 8177419 | 45756 | 150430556 | A/— | A = 0.00 | A = 0.00 | | | |
| | | | | — = 1.00 | — = 1.00 | | | |
| 8177420 | 45826 | 150430626 | T/C | T = 1.00 | T = 1.00 | | | |
| | | | | C = 0.00 | C = 0.00 | | | |
| 8177421 | 45974 | 150430774 | C/G | C = 0.07 | C = 0.02 | | | |
| | | | | G = 0.93 | G = 0.98 | | | |
| 4958872 | 46044 | 150430844 | T/C | T = 0.81 | T = 0.81 | 0.9167 | 0.98 | T |
| | | | | C = 0.19 | C = 0.19 | | | |
| 3792796 | 46200 | 150431000 | C/G | C = 0.40 | C = 0.41 | 0.5382 | 1.08 | C |
| | | | | G = 0.60 | G = 0.59 | | | |
| 8177422 | 46218 | 150431018 | A/G | A = 1.00 | A = 1.00 | | | |
| | | | | G = 0.00 | G = 0.00 | | | |
| 8177423 | 46221 | 150431021 | C/T | C = 1.00 | C = 1.00 | | | |
| | | | | T = 0.00 | T = 0.00 | | | |
| 4958434 | 46280 | 150431080 | C/T | C = 0.86 | C = 0.83 | 0.1864 | 0.79 | T |
| | | | | T = 0.14 | T = 0.17 | | | |
| 8177424 | 46330 | 150431138 | —/ | — = 1.00 | — = 0.98 | | | |
| | | | GAGTCCTGG | GAGTCCTGG = 0.00 | GAGTCCTGG = 0.02 | | | |
| 8177425 | 46583 | 150431383 | T/C | T = 0.02 | T = 0.03 | 0.5080 | 1.30 | T |
| | | | | C = 0.98 | C = 0.97 | | | |
| 8177426 | 46650 | 150431450 | A/G | A = 0.31 | A = 0.36 | 0.0447 | 1.29 | A |
| | | | | G = 0.69 | G = 0.64 | | | |
| 8177427 | 46721 | 150431521 | A/G | A = 0.26 | A = 0.32 | 0.0296 | 1.33 | A |
| | | | | G = 0.74 | G = 0.68 | | | |
| 8177429 | 46808 | 150431608 | G/C | G = 0.91 | G = 0.95 | 0.0132 | 1.89 | G |
| | | | | C = 0.09 | C = 0.05 | | | |
| 6889737 | 47242 | 150432042 | C/A | C = 1.00 | C = 1.00 | | | |
| | | | | A = 0.00 | A = 0.00 | | | |
| 3792795 | 47512 | 150432312 | G/A | G = 0.89 | G = 0.97 | 0.0000 | 3.53 | G |
| | | | | A = 0.11 | A = 0.03 | | | |
| 8177430 | 47600 | 150432400 | T/C | T = 0.01 | T = 0.01 | | | |
| | | | | C = 0.99 | C = 0.99 | | | |
| 8177431 | 47706 | 150432506 | A/G | A = 0.71 | A = 0.70 | 0.6316 | 0.94 | G |
| | | | | G = 0.29 | G = 0.30 | | | |
| 4958873 | 47806 | 150432606 | A/G | A = 0.31 | A = 0.38 | 0.0279 | 1.32 | A |
| | | | | G = 0.69 | G = 0.62 | | | |
| 8177432 | 47978 | 150432778 | T/G | T = 1.00 | T = 1.00 | | | |
| | | | | G = 0.00 | G = 0.00 | | | |
| 8177433 | 48021 | 150432821 | T/C | T = 0.26 | T = 0.23 | 0.2375 | 0.85 | C |
| | | | | C = 0.74 | C = 0.77 | | | |
| 8177434 | 48025 | 150432825 | G/A | G = 0.00 | G = 0.00 | | | |
| | | | | A = 1.00 | A = 1.00 | | | |
| 8177435 | 48093 | 150432893 | T/G | T = 0.81 | T = 0.79 | 0.4469 | 0.89 | G |
| | | | | G = 0.19 | G = 0.21 | | | |
| 3763011 | 48413 | 150433213 | T/C | T = 0.01 | T = 0.02 | | | |
| | | | | C = 0.99 | C = 0.98 | | | |
| 8177436 | 48933 | 150433733 | C/T | C = 0.00 | C = 0.00 | | | |
| | | | | T = 1.00 | T = 1.00 | | | |
| 8177437 | 49097 | 150433897 | C/G | C = 0.17 | C = 0.07 | ˜0.00001 | 0.38 | G |
| | | | | G = 0.83 | G = 0.93 | | | |
| 4958874 | 49105 | 150433905 | T/C | T = 0.44 | T = 0.49 | 0.0880 | 1.24 | T |
| | | | | C = 0.56 | C = 0.51 | | | |
| 8177439 | 49570 | 150434370 | G/A | G = | G = 1.00 | | | |
| | | | | A = | A = 0.00 | | | |
| 8177440 | 49591 | 150434391 | C/T | C = 1.00 | C = 1.00 | | | |
| | | | | T = 0.00 | T = 0.00 | | | |
| 8177441 | 49704 | 150434504 | G/C | G = 1.00 | G = 1.00 | | | |
| | | | | C = 0.00 | C = 0.00 | | | |
| 8177442 | 49705 | 150434505 | A/T | A = 1.00 | A = 1.00 | | | |
| | | | | T = 0.00 | T = 0.00 | | | |
| 8177443 | 49798 | 150434598 | C/T | C = 1.00 | C = 1.00 | | | |
| | | | | T = 0.00 | T = 0.00 | | | |
| 869975 | 50082 | 150434882 | A/G | A = 0.37 | A = 0.25 | ˜0.00001 | 0.57 | G |
| | | | | G = 0.63 | G = 0.75 | | | |
| 869976 | 50147 | 150434947 | G/A | G = 0.00 | G = 0.00 | | | |
| | | | | A = 1.00 | A = 1.00 | | | |
| 8177444 | 50356 | 150435156 | A/T | A = 1.00 | A = 1.00 | | | |
| | | | | T = 0.00 | T = 0.00 | | | |
| 8177445 | 50725 | 150435525 | T/C | T = 1.00 | T = 1.00 | | | |
| | | | | C = 0.00 | C = 0.00 | | | |
| 7721469 | 50968 | 150435768 | C/A | C = 0.00 | C = 0.00 | | | |
| | | | | A = 1.00 | A = 1.00 | | | |
| 8177446 | 51029 | 150435829 | A/C | A = 1.00 | A = 1.00 | | | |
| | | | | C = 0.00 | C = 0.00 | | | |
| 7704191 | 51086 | 150435886 | T/C | T = 1.00 | T = 1.00 | | | |
| | | | | C = 0.00 | C = 0.00 | | | |
| 8177447 | 51166 | 150435966 | T/C | T = 0.27 | T = 0.35 | 0.0030 | 1.50 | T |
| | | | | C = 0.73 | C = 0.65 | | | |
| 11548 | 51493 | 150436293 | C/T | C = 0.90 | C = 0.95 | 0.0002 | 2.40 | C |
| | | | | T = 0.10 | T = 0.05 | | | |
| 2230303 | 51539 | 150436339 | G/T | G = 0.00 | G = 0.01 | | | |
| | | | | T = 1.00 | T = 0.99 | | | |
| 7722386 | 51562 | 150436362 | G/A | G = 0.00 | G = 0.00 | | | |
| | | | | A = 1.00 | A = 1.00 | | | |
| 8177448 | 51645 | 150436445 | G/A | G = 0.99 | G = 0.99 | | | |
| | | | | A = 0.01 | A = 0.01 | | | |
| 8177449 | 51649 | 150436449 | T/C | T = 0.17 | T = 0.14 | 0.2009 | 0.80 | C |
| | | | | C = 0.83 | C = 0.86 | | | |
| 2070593 | 51650 | 150436450 | T/C | T = 0.31 | T = 0.28 | 0.2540 | 0.86 | C |
| | | | | C = 0.69 | C = 0.72 | | | |
| 8177450 | 51656 | 150436456 | G/A | G = 0.00 | G = 0.00 | | | |
| | | | | A = 1.00 | A = 1.00 | | | |
| 8177451 | 51657 | 150436457 | T/C | T = 0.00 | T = 0.00 | | | |
| | | | | C = 1.00 | C = 1.00 | | | |
| 8177452 | 52009 | 150436809 | G/A | G = 0.00 | G = 0.00 | | | |
| | | | | A = 1.00 | A = 1.00 | | | |
| 8177453 | 52143 | 150436943 | A/C | A = 0.00 | A = 0.00 | | | |
| | | | | C = 1.00 | C = 1.00 | | | |
| 8177454 | 52349 | 150437149 | G/C | G = 0.99 | G = 0.98 | | | |
| | | | | C = 0.01 | C = 0.02 | | | |
| 3763010 | 52421 | 150437221 | C/T | C = 0.76 | C = 0.80 | 0.1337 | 1.30 | C |
| | | | | T = 0.24 | T = 0.20 | | | |
| 8177455 | 52532 | 150437332 | A/G | A = 0.00 | A = 0.00 | | | |
| | | | | G = 1.00 | G = 1.00 | | | |
| 8177456 | 52682 | 150437482 | A/G | A = 0.45 | A = 0.50 | 0.1098 | 1.22 | A |
| | | | | G = 0.55 | G = 0.50 | | | |
| 736775 | 53058 | 150437858 | T/C | T = 0.56 | T = 0.59 | 0.3945 | 1.12 | T |
| | | | | C = 0.44 | C = 0.41 | | | |
| 2277940 | 53187 | 150437987 | T/C | T = 0.92 | T = 0.97 | 0.0004 | 2.75 | T |
| | | | | C = 0.08 | C = 0.03 | | | |
| 8177458 | 53377 | 150438177 | G/A | G = 0.98 | G = 0.98 | 0.9445 | 1.03 | G |
| | | | | A = 0.02 | A = 0.02 | | | |
| 8177834 | 53699 | 150438499 | G/A | G = 0.93 | G = 0.89 | 0.0113 | 0.58 | A |
| | | | | A = 0.07 | A = 0.11 | | | |
| 3924 | 53845 | 150438645 | A/G | A = 0.75 | A = 0.74 | 0.7350 | 0.95 | G |
| | | | | G = 0.25 | G = 0.26 | | | |
| 2233312 | 53920 | 150438720 | A/G | A = 0.00 | A = 0.00 | | | |
| | | | | G = 1.00 | G = 1.00 | | | |
| 2233311 | 53929 | 150438729 | T/G | T = 0.15 | T = 0.20 | 0.0106 | 1.49 | T |
| | | | | G = 0.85 | G = 0.80 | | | |
| 2233310 | 55473 | 150440273 | T/C | T = 0.00 | T = 0.00 | | | T |
| | | | | C = 1.00 | C = 1.00 | | | |
| 2233309 | 55690 | 150440490 | A/G | A = 0.00 | A = 0.00 | | | |
| | | | | G = 1.00 | G = 1.00 | | | |
| 4958875 | 55850 | 150440650 | A/G | A = 0.56 | A = 0.53 | 0.2131 | 0.86 | G |
| | | | | G = 0.44 | G = 0.47 | | | |
| 2233308 | 56761 | 150441561 | C/T | C = 1.00 | C = 1.00 | | | |
| | | | | T = 0.00 | T = 0.00 | | | |
| 2233307 | 56840 | 150441640 | C/G | C = 0.00 | C = 0.00 | | | |
| | | | | G = 1.00 | G = 1.00 | | | |
| 2233306 | 57000 | 150441800 | C/T | C = 1.00 | C = 1.00 | | | |
| | | | | T = 0.00 | T = 0.00 | | | |
| 2233305 | 57116 | 150441916 | T/G | T = | T = 0.27 | | | |
| | | | | G = | G = 0.73 | | | |
| 2233304 | 58419 | 150443219 | T/G | T = 0.00 | T = 0.01 | | | |
| | | | | G = 1.00 | G = 0.99 | | | |
| 2233303 | 58420 | 150443220 | C/T | C = 1.00 | C = 1.00 | | | |
| | | | | T = 0.00 | T = 0.00 | | | |
| 2233302 | 58808 | 150443608 | G/C | G = 0.98 | G = 0.87 | ˜0.00001 | 0.14 | C |
| | | | | C = 0.02 | C = 0.13 | | | |
| 2287719 | 58906 | 150443706 | A/G | A = 0.00 | A = 0.00 | | | |
| | | | | G = 1.00 | G = 1.00 | | | |
| 2287720 | 59048 | 150443848 | C/T | C = 0.39 | C = 0.42 | 0.2852 | 1.14 | C |
| | | | | T = 0.61 | T = 0.58 | | | |
| 7727034 | 59187 | 150443987 | C/G | C = 0.24 | C = 0.30 | 0.0374 | 1.36 | C |
| | | | | G = 0.76 | G = 0.70 | | | |
| 7727250 | 59361 | 150444161 | C/T | C = 0.33 | C = 0.43 | 0.0003 | 1.57 | C |
| | | | | T = 0.67 | T = 0.57 | | | |
| 7709800 | 61218 | 150446018 | G/A | G = 1.00 | G = 1.00 | | | |
| | | | | A = 0.00 | A = 0.00 | | | |
| 3840312 | 61700 | 150446500 | G/— | G = 0.54 | G = 0.50 | 0.1346 | 0.84 | — |
| | | | | — = 0.46 | — = 0.50 | | | |
| 2287721 | 62290 | 150447090 | G/A | G = 1.00 | G = 1.00 | | | |
| | | | | A = 0.00 | A = 0.00 | | | |
| 6875293 | 62596 | 150447396 | T/C | T = 0.00 | T = 0.00 | | | |
| | | | | C = 1.00 | C = 1.00 | | | |
| 3805434 | 64049 | 150448849 | G/C | G = 0.93 | G = 0.87 | 0.0009 | 0.51 | C |
| | | | | C = 0.07 | C = 0.13 | | | |
| 2080982 | 66077 | 150450877 | G/T | G = 0.02 | G = 0.01 | | | |
| | | | | T = 0.98 | T = 0.99 | | | |
| 2080983 | 66079 | 150450879 | G/T | G = 1.00 | G = 1.00 | | | |
| | | | | T = 0.00 | T = 0.00 | | | |
| 2287722 | 66086 | 150450886 | T/C | T = 0.00 | T = 0.00 | | | |
| | | | | C = 1.00 | C = 1.00 | | | |
| 2233301 | 66115 | 150450915 | T/G | T = 0.00 | T = 0.00 | | | |
| | | | | G = 1.00 | G = 1.00 | | | |
| 2233300 | 66150 | 150450950 | C/G | C = 0.00 | C = 0.00 | | | |
| | | | | G = 1.00 | G = 1.00 | | | |
| 4958876 | 66475 | 150451275 | C/A | C = | C = 0.95 | | | |
| | | | | A = | A = 0.05 | | | |
| 2233299 | 69177 | 150453977 | A/G | A = 0.48 | A = 0.49 | 0.7917 | 1.03 | A |
| | | | | G = 0.52 | G = 0.51 | | | |
| 2233298 | 69210 | 150454010 | A/G | A = 0.00 | A = 0.00 | | | |
| | | | | G = 1.00 | G = 1.00 | | | |
| 2287723 | 69312 | 150454112 | T/G | T = 1.00 | T = 1.00 | | | |
| | | | | G = 0.00 | G = 0.00 | | | |
| 2161359 | 70244 | 150455044 | A/G | A = 0.53 | A = 0.51 | 0.5813 | 0.94 | G |
| | | | | G = 0.47 | G = 0.49 | | | |
| 7734456 | 70882 | 150455682 | G/C | G = 0.53 | G = 0.60 | 0.0188 | 1.32 | G |
| | | | | C = 0.47 | C = 0.40 | | | |
| 4292439 | 71905 | 150456705 | T/C | T = 0.87 | T = 0.84 | 0.1343 | 0.77 | C |
| | | | | C = 0.13 | C = 0.16 | | | |
| 4958878 | 72294 | 150457094 | A/T | A = | A = 0.12 | | | |
| | | | | T = | T = 0.88 | | | |
| 6862024 | 72581 | 150457381 | A/G | A = 0.80 | A = 0.78 | 0.4512 | 0.89 | G |
| | | | | G = 0.20 | G = 0.22 | | | |
| 3834819 | 72786 | 150457589 | —/CA | — = 0.00 | — = 0.00 | | | |
| | | | | CA = 1.00 | CA = 1.00 | | | |
| 2233297 | 72950 | 150457750 | G/A | G = 0.04 | G = 0.05 | 0.3215 | 1.32 | G |
| | | | | A = 0.96 | A = 0.95 | | | |
| 2233296 | 73106 | 150457906 | G/A | G = 1.00 | G = 1.00 | | | |
| | | | | A = 0.00 | A = 0.00 | | | |
| 2233295 | 73162 | 150457962 | T/C | T = 0.00 | T = 0.00 | | | |
| | | | | C = 1.00 | C = 1.00 | | | |
| 2233294 | 73273 | 150458073 | G/T | G = 0.24 | G = 0.23 | 0.7059 | 0.95 | T |
| | | | | T = 0.76 | T = 0.77 | | | |
| 7713028 | 74131 | 150458931 | G/A | G = 0.9 | G = 0.85 | 0.0238 | 0.66 | A |
| | | | | A = 0.1 | A = 0.15 | | | |
| 7713223 | 74406 | 150459206 | T/C | T = 0.93 | T = 0.88 | 0.0047 | 0.56 | C |
| | | | | C = 0.07 | C = 0.12 | | | |
| 7713567 | 74665 | 150459465 | C/T | C = 0.61 | C = 0.63 | 0.4135 | 1.10 | C |
| | | | | T = 0.39 | T = 0.37 | | | |
| 888989 | 74740 | 150459540 | T/C | T = 0.90 | T = 0.86 | 0.0352 | 0.67 | C |
| | | | | C = 0.10 | C = 0.14 | | | |
| 2233293 | 75382 | 150460182 | A/G | A = 0.00 | A = 0.00 | | | |
| | | | | G = 1.00 | G = 1.00 | | | |
| 3749657 | 75400 | 150460200 | G/T | G = 1.00 | G = 1.00 | | | |
| | | | | T = 0.00 | T = 0.00 | | | |
| 2233292 | 75460 | 150460260 | G/A | G = | G = 1.00 | | | |
| | | | | A = | A = 0.00 | | | |
| 2112635 | 75863 | 150460663 | C/T | C = 0.35 | C = 0.36 | 0.7381 | 1.04 | C |
| | | | | T = 0.65 | T = 0.64 | | | |
| 871269 | 76098 | 150460898 | T/C | T = 0.47 | T = 0.42 | 0.0796 | 0.81 | C |
| | | | | C = 0.53 | C = 0.58 | | | |
| 3792794 | 78432 | 150463232 | A/G | A = 0.21 | A = 0.10 | ˜0.00001 | 0.41 | G |
| | | | | G = 0.79 | G = 0.90 | | | |
| 6579837 | 78604 | 150463404 | G/T | G = 0.89 | G = 0.91 | 0.1630 | 1.33 | G |
| | | | | T = 0.11 | T = 0.09 | | | |
| 3805433 | 79190 | 150463990 | C/G | C = 0.28 | C = 0.28 | 0.9484 | 0.99 | C |
| | | | | G = 0.72 | G = 0.72 | | | |
| 5872186 | 79870 | 150464670 | —/A | — = 0.33 | — = 0.35 | 0.5526 | 1.08 | — |
| | | | | A = 0.67 | A = 0.65 | | | |
| 2233291 | 79928 | 150464728 | G/C | G = 1.00 | G = 1.00 | | | |
| | | | | C = 0.00 | C = 0.00 | | | |
| 2233290 | 80213 | 150465013 | G/C | G = 0.14 | G = 0.13 | 0.8704 | 0.97 | C |
| | | | | C = 0.86 | C = 0.87 | | | |
| 2233289 | 80227 | 150465027 | C/T | C = 1.00 | C = 0.99 | | | |
| | | | | T = 0.00 | T = 0.010 | | | |
| 4958435 | 81994 | 150466794 | T/G | T = 0.68 | T = 0.63 | 0.0549 | 0.79 | G |
| | | | | G = 0.32 | G = 0.37 | | | |
| 4958880 | 82187 | 150466987 | C/A | C = 0.75 | C = 0.84 | 0.0002 | 1.76 | C |
| | | | | A = 0.25 | A = 0.16 | | | |
| 1422673 | 82698 | 150467498 | T/C | T = 0.45 | T = 0.32 | ˜0.00001 | 0.59 | C |
| | | | | C = 0.55 | C = 0.68 | | | |
| 2042234 | 82841 | 150467641 | A/G | A = 0.91 | A = 0.93 | 0.1485 | 1.37 | A |
| | | | | G = 0.09 | G = 0.07 | | | |
| 3805432 | 83214 | 150468014 | A/G | A = 0.04 | A = 0.07 | 0.0188 | 1.93 | A |
| | | | | G = 0.96 | G = 0.93 | | | |
| 3805431 | 83249 | 150468049 | T/C | T = 0.06 | T = 0.02 | | | |
| | | | | C = 0.94 | C = 0.98 | | | |
| 2233288 | 83485 | 150468285 | T/C | T = 0.00 | T = 0.00 | | | |
| | | | | C = 1.00 | C = 1.00 | | | |
| 2233287 | 83807 | 150468607 | C/T | C = 0.90 | C = 0.92 | 0.2555 | 1.32 | C |
| | | | | T = 0.10 | T = 0.08 | | | |
| 3815720 | 83907 | 150468707 | G/A | G = 0.99 | G = 0.97 | | | |
| | | | | A = 0.01 | A = 0.03 | | | |
| 3792792 | 84216 | 150469016 | A/G | A = 1.00 | A = 0.99 | | | |
| | | | | G = 0.00 | G = 0.01 | | | |
| 3792791 | 84656 | 150469456 | A/G | A = 0.00 | A = 0.00 | | | |
| | | | | G = 1.00 | G = 1.00 | | | |
| 2303018 | 85448 | 150470248 | G/A | G = 1.00 | G = 1.00 | | | |
| | | | | A = 0.00 | A = 0.00 | | | |
| 3792790 | 85881 | 150470681 | G/T | G = 0.49 | G = 0.49 | 0.9686 | 1.00 | G |
| | | | | T = 0.51 | T = 0.51 | | | |
| 4958436 | 86539 | 150471339 | T/C | T = 0.88 | T = 0.92 | 0.0083 | 1.76 | T |
| | | | | C = 0.12 | C = 0.08 | | | |
| 2233286 | 86796 | 150471596 | T/C | T = 0.00 | T = 0.00 | | | |
| | | | | C = 1.00 | C = 1.00 | | | |
| 2233285 | 87057 | 150471857 | G/A | G = 1.00 | C = 1.00 | | | |
| | | | | A = 0.00 | A = 0.00 | | | |
| 7732451 | 87922 | 150472722 | T/C | T = 0.90 | T = 0.92 | 0.2349 | 1.29 | T |
| | | | | C = 0.10 | C = 0.08 | | | |
| 2233284 | 88098 | 150472898 | T/C | T = 0.00 | T = 0.00 | | | |
| | | | | C = 1.00 | C = 1.00 | | | |
| 1422674 | 89319 | 150474119 | G/T | G = | G = | | | |
| | | | | T = | T = | | | |
| 3792789 | 89678 | 150474478 | C/T | C = 0.42 | C = 0.42 | 0.9873 | 1.00 | C |
| | | | | T = 0.58 | T = 0.58 | | | |
| 4562032 | 90026 | 150474826 | C/A | C = | C = 1.00 | | | |
| | | | | A = | A = 0.00 | | | |
| 6865077 | 90033 | 150474833 | G/A | G = 1.00 | G = 1.00 | | | |
| | | | | A = 0.00 | A = 0.00 | | | |
| 1559126 | 90114 | 150474914 | C/G | C = 1.00 | C = 1.00 | | | |
| | | | | G = 0.00 | G = 0.00 | | | |
| 3792788 | 90326 | 150475126 | T/C | T = 0.00 | T = 0.00 | | | |
| | | | | C = 1.00 | C = 1.00 | | | |
| 1559127 | 90463 | 150475263 | T/C | T = 0.94 | T = 0.94 | 0.6080 | 1.14 | T |
| | | | | C = 0.06 | C = 0.06 | | | |
| 3792786 | 90548 | 150475348 | A/G | A = 0.98 | A = 0.99 | | | |
| | | | | G = 0.02 | G = 0.01 | | | |
| 6880110 | 90800 | 150475600 | A/G | A = 0.91 | A = 0.92 | 0.5433 | 1.14 | A |
| | | | | G = 0.09 | G = 0.08 | | | |
| 6861227 | 90838 | 150475638 | G/T | G = 0.26 | G = 0.23 | 0.1843 | 0.83 | T |
| | | | | T = 0.74 | T = 0.77 | | | |
| 3805430 | 91400 | 150476200 | C/G | C = 0.02 | C = 0.05 | | | |
| | | | | G = 0.98 | G = 0.95 | | | |
| 1862364 | 92086 | 150476886 | A/G | A = 0.91 | A = 0.92 | 0.5575 | 1.13 | A |
| | | | | G = 0.09 | G = 0.08 | | | |
| 4958881 | 93946 | 150478746 | T/C | T = 0.91 | T = 0.91 | 0.9701 | 1.01 | T |
| | | | | C = 0.09 | C = 0.09 | | | |
| 3792785 | 95360 | 150480160 | A/G | A = 0.92 | A = 0.93 | 0.5448 | 1.16 | A |
| | | | | G = 0.08 | G = 0.07 | | | |
| 6869605 | 96576 | 150481376 | A/C | A = 0.90 | A = 0.89 | 0.7189 | 0.93 | C |
| | | | | C = 0.10 | C = 0.11 | | | |
| 6870205 | 96721 | 150481521 | A/G | A = 0.00 | A = 0.07 | | | |
| | | | | G = 1.00 | G = 0.93 | | | |
| 4246047 | 98316 | 150483116 | T/A | T = 0.94 | T = 0.96 | 0.2613 | 1.37 | T |
| | | | | A = 0.06 | A = 0.04 | | | |
| 4958882 | 98497 | 150483297 | C/G | C = 0.88 | C = 0.89 | 0.7723 | 1.06 | C |
| | | | | G = 0.12 | G = 0.11 | | | |
| 3792784 | 99382 | 150484182 | C/T | C = 0.00 | C = 0.04 | | | |
| | | | | T = 1.00 | T = 0.96 | | | |
| 3792783 | 99442 | 150484242 | T/C | T = 0.85 | T = 0.87 | 0.2391 | 1.22 | T |
| | | | | C = 0.15 | C = 0.13 | | | |
| 5872188 | 99764 | 150484565 | —/AG | — = 0.90 | — = 0.91 | 0.5908 | 1.11 | — |
| | | | | AG = 0.10 | AG = 0.09 | | | |

Allelotyping results were considered particularly significant with a calculated p-value of less than or equal to 0.05 for allelotype results. These values are indicated in bold. The allelotyping p-values were plotted in FIG. 4. The position of each SNP on the chromosome is presented on the x-axis. The y-axis gives the negative logarithm (base 10) of the p-value comparing the estimated allele in the case group to that of the control group. The minor allele frequency of the control group for each SNP designated by an X or other symbol on the graphs in FIG. 4 can be determined by consulting Table 27. For example, the left-most X on the left graph is at position 150385031. By proceeding down the Table from top to bottom and across the graphs from left to right the allele frequency associated with each symbol shown can be determined.

To aid the interpretation, multiple lines have been added to the graph. The broken horizontal lines are drawn at two common significance levels, 0.05 and 0.01. The vertical broken lines are drawn every 20 kb to assist in the interpretation of distances between SNPs. Two other lines are drawn to expose linear trends in the association of SNPs to the disease. The light gray line (or generally bottom-most curve) is a nonlinear smoother through the data points on the graph using a local polynomial regression method (W. S. Cleveland, E. Grosse and W. M. Shyu (1992) Local regression models. Chapter 8 of Statistical Models in S eds J. M. Chambers and T. J. Hastie, Wadsworth & Brooks/Cole.). The black line provides a local test for excess statistical significance to identify regions of association. This was created by use of a 10 kb sliding window with 1 kb step sizes. Within each window, a chi-square goodness of fit test was applied to compare the proportion of SNPs that were significant at a test wise level of 0.01, to the proportion that would be expected by chance alone (0.05 for the methods used here). Resulting p-values that were less than 104 were truncated at that value.

Finally, the exons and introns of the genes in the covered region are plotted below each graph at the appropriate chromosomal positions. The gene boundary is indicated by the broken horizontal line. The exon positions are shown as thick, unbroken bars. An arrow is place at the 3′ end of each gene to show the direction of transcription.

### Example 10

### In Vitro Production of Target Polypeptides

cDNA is cloned into a pIVEX 2.3-MCS vector (Roche Biochem) using a directional cloning method. A cDNA insert is prepared using PCR with forward and reverse primers having 5′ restriction site tags (in frame) and 5-6 additional nucleotides in addition to 3′ gene-specific portions, the latter of which is typically about twenty to about twenty-five base pairs in length. A Sal I restriction site is introduced by the forward primer and a Sma I restriction site is introduced by the reverse primer. The ends of PCR products are cut with the corresponding restriction enzymes (i.e., Sal I and Sma I) and the products are gel-purified. The pIVEX 2.3-MCS vector is linearized using the same restriction enzymes, and the fragment with the correct sized fragment is isolated by gel-purification. Purified PCR product is ligated into the linearized pIVEX 2.3-MCS vector and E. coli cells transformed for plasmid amplification. The newly constructed expression vector is verified by restriction mapping and used for protein production.

E. coli lysate is reconstituted with 0.25 ml of Reconstitution Buffer, the Reaction Mix is reconstituted with 0.8 ml of Reconstitution Buffer; the Feeding Mix is reconstituted with 10.5 ml of Reconstitution Buffer; and the Energy Mix is reconstituted with 0.6 ml of Reconstitution Buffer. 0.5 ml of the Energy Mix was added to the Feeding Mix to obtain the Feeding Solution. 0.75 ml of Reaction Mix, 50 μl of Energy Mix, and 10 μg of the template DNA is added to the E. coli lysate.

Using the reaction device (Roche Biochem), 1 ml of the Reaction Solution is loaded into the reaction compartment. The reaction device is turned upside-down and 10 ml of the Feeding Solution is loaded into the feeding compartment. All lids are closed and the reaction device is loaded into the RTS500 instrument. The instrument is run at 30° C. for 24 hours with a stir bar speed of 150 rpm. The pIVEX 2.3 MCS vector includes a nucleotide sequence that encodes six consecutive histidine amino acids on the C-terminal end of the target polypeptide for the purpose of protein purification. Target polypeptide is purified by contacting the contents of reaction device with resin modified with Ni²⁺ ions. Target polypeptide is eluted from the resin with a solution containing free Ni²⁺ ions.

### Example 11

### Cellular Production of Target Polypeptides

Nucleic acids are cloned into DNA plasmids having phage recombination cites and target polypeptides are expressed therefrom in a variety of host cells. Alpha phage genomic DNA contains short sequences known as attP sites, and E. coli genomic DNA contains unique, short sequences known as attB sites. These regions share homology, allowing for integration of phage DNA into E. coli via directional, site-specific recombination using the phage protein Int and the E. coli protein IHF. Integration produces two new aft sites, L and R, which flank the inserted prophage DNA. Phage excision from E. coli genomic DNA can also be accomplished using these two proteins with the addition of a second phage protein, Xis. DNA vectors have been produced where the integration/excision process is modified to allow for the directional integration or excision of a target DNA fragment into a backbone vector in a rapid in vitro reaction (Gateway™ Technology (Invitrogen, Inc.)).

A first step is to transfer the nucleic acid insert into a shuttle vector that contains attL sites surrounding the negative selection gene, ccdB (e.g. pENTER vector, Invitrogen, Inc.). This transfer process is accomplished by digesting the nucleic acid from a DNA vector used for sequencing, and to ligate it into the multicloning site of the shuttle vector, which will place it between the two attL sites while removing the negative selection gene ccdB. A second method is to amplify the nucleic acid by the polymerase chain reaction (PCR) with primers containing attB sites. The amplified fragment then is integrated into the shuttle vector using Int and IHF. A third method is to utilize a topoisomerase-mediated process, in which the nucleic acid is amplified via PCR using gene-specific primers with the 5′ upstream primer containing an additional CACC sequence (e.g., TOPO® expression kit (Invitrogen, Inc.)). In conjunction with Topoisomerase I, the PCR amplified fragment can be cloned into the shuttle vector via the attL sites in the correct orientation.

Once the nucleic acid is transferred into the shuttle vector, it can be cloned into an expression vector having attR sites. Several vectors containing attR sites for expression of target polypeptide as a native polypeptide, N-fusion polypeptide, and C-fusion polypeptides are commercially available (e.g., pDEST (Invitrogen, Inc.)), and any vector can be converted into an expression vector for receiving a nucleic acid from the shuttle vector by introducing an insert having an attR site flanked by an antibiotic resistant gene for selection using the standard methods described above. Transfer of the nucleic acid from the shuttle vector is accomplished by directional recombination using Int, IHF, and Xis (LR clonase). Then the desired sequence can be transferred to an expression vector by carrying out a one hour incubation at room temperature with Int, IHF, and Xis, a ten minute incubation at 37° C. with proteinase K, transforming bacteria and allowing expression for one hour, and then plating on selective media. Generally, 90% cloning efficiency is achieved by this method. Examples of expression vectors are pDEST 14 bacterial expression vector with att7 promoter, pDEST 15 bacterial expression vector with a T7 promoter and a N-terminal GST tag, pDEST 17 bacterial vector with a T7 promoter and a N-terminal polyhistidine affinity tag, and pDEST 12.2 mammalian expression vector with a CMV promoter and neo resistance gene. These expression vectors or others like them are transformed or transfected into cells for expression of the target polypeptide or polypeptide variants. These expression vectors are often transfected, for example, into murine-transformed a adipocyte cell line 3T3-L1, (ATCC), human embryonic kidney cell line 293, and rat cardiomyocyte cell line H9C2.

Representative Nucleotide and Amino Acid Sequences

Following is a genomic nucleotide sequence for a CTEP region. The genomic nucleotide sequence is set forth in SEQ ID NO: 1. The following nucleotide representations are used throughout: “A” or “a” is adenosine, adenine, or adenylic acid; “C” or “c” is cytidine, cytosine, or cytidylic acid; “G” or “g” is guanosine, guanine, or guanylic acid; “T” or “t” is thymidine, thymine, or thymidylic acid; and “I” or “i” is inosine, hypoxanthine, or inosinic acid. Exons are indicated in italicized lower case type, introns are depicted in normal text lower case type, and polymorphic sites are depicted in bold upper case type. SNPs are designated by the following convention: “R” represents A or G, “M” represents A or C; “W” represents A or T; “Y” represents C or T; “S” represents C or G; “K” represents G or T; “V” represents A, C or G; “H” represents A, C, or T; “D” represents A, G, or T; “B” represents C, G, or T; and “N” represents A, G, C, or T.

>16: 56742951-56841200

| | |
|---|---|
| 1 | tgtctaaaaa ttattatggc tgaaacatac ttatcatttg |
| | ttttctgtag tagaaaatgg |
| 61 | cataatatga gaaggcctac tttgtaatag ttgtttgctt |
| | tacttaatat ttttcaaatc |
| 121 | agccatctgt gtgaagactc aatatcaagt taggccttta |
| | aaagggggaa aataattcaa |
| 181 | aaccactggc agctgtggtt ttccRgcctt ttctattggt |
| | tgaactctgt aataaatgca |
| 241 | ggaccctaca tatacaaaag gtactgtttt ccaaattcac |
| | gtatgtatta gcatttacct |
| 301 | ttaaagttct catccgtcag aaaaacggtg aacacagcat |
| | tcagtatatt tattttggtt |
| 361 | tagttacaca gtgtcaaaaa gttctgatgt atatggataa |
| | gtagatgcac tttttgctgt |
| 421 | tttgtttgtt taaacagctt gctttaatta tctcaagcta |
| | cttggcagga acacctttat |
| 481 | tccaggtttc gcataatcag ttattctggc aagagtagat |
| | taacacgtta gtgatctcca |
| 541 | atagacagat tatatgttaa tattatttga ggccattatt |
| | tttaagtaca atttagacgg |
| 601 | agtttcaaat agattttaaa aatgagattt gtataaaaga |
| | gtgtgcagtc cctatggtgt |
| 661 | taaaatcact gatatggagg agaagagggg ttggaattgt |
| | gacactgtgg cagcagatga |
| 721 | ccttggacaa gtcagttttt cagactttcc tcccttaaaa |
| | tagggatact aaatctcaca |
| 781 | gggtggttgt gtataatgtc acccacacgc catctcctct |
| | ataactgcat ttgtatgcca |
| 841 | ggaaattgtc tgtgaaggct tcagtttaga atagcttcat |
| | attattttgg ggcactgctg |
| 901 | ggtgattttg gttaaggctg gccttgggag ttctctgagt |
| | tttctctgaa accaaactca |
| 961 | atttggttgt aggttccctg tttgctgttt gttgctttta |
| | gtaataggag tcagtcagtt |
| 1021 | tacctgggag ttaagtttgg actggagata atttgccatt |
| | cttttttttt tttttttttt |
| 1081 | ttttttttga gatagggtct cactctgtcg cccaggctgg |
| | agtgcagtga catgatcttg |
| 1141 | gctcactgca acctctgcct ccctggttca agcgattccc |
| | ctgcctcagc ctcctgagta |
| 1201 | gtttgggtta caggcacaag ccaccacgcc cagctaattt |
| | ttgcattttt agtagagatg |
| 1261 | gggtttcacc atgttggcca ggctggtctc gaacccctga |
| | cctcaggtga tctgcccgcc |
| 1321 | ttggcctccc acagtgcttg gattacaggc atgagccacc |
| | atgcccagcc gataatttgc |
| 1381 | cattctgtat ttatttgcgt gttagagacc agttaaagtt |
| | aagacacttt ctaaatagaa |
| 1441 | gtcttgtata tggggctttt gagaaagtgt tttcccgtga |
| | tgctgggggg aaacacacat |
| 1501 | attcaaatta cttgtataaa acagtttatt ctgtgtttag |
| | gaacttgtag gggtgaaatg |
| 1561 | ctgacctgtg ttactctttc tttccatgat cagcRtccag |
| | aggaccagag gttaatttat |
| 1621 | tctgggaagc tgttgttgga tcaccaatgt ctcagggact |
| | tgcttccaaa ggtacatcac |
| 1681 | ttacacatta acttctgaat gtttttaagc actcaccagg |
| | ttaggttcag gtccttaagt |
| 1741 | accttcgtat tacttttagc aggaaaaacg gcatgttttg |
| | catctggtgt gcaatgtgaa |
| 1801 | gagtccttca aaaatgccag aaatcaacgc caaggtgtgt |
| | ctgcctcttc atgatggtaa |
| 1861 | caatttgtat cattcagact ttcaggggag taataaaaga |
| | agttggataa aacgtttatt |
| 1921 | gagagactgt atggtgttat taccaagtaa tattttgttt |
| | ctttcctagg aattagaaac |
| 1981 | tgtattatca acacaaagtg atgagttaat agtagcagat |
| | tgttccgtta gtgctgtagt |
| 2041 | agaaaggtgg agttacatat tgcactatct tatgggatat |
| | gatttgatct atggttactg |
| 2101 | tgtatctaga ttactgaact aacactaatc gggtttttat |
| | tgaatttagc acatagcaag |
| 2161 | tttcttcaaa ggagtcacta atttttataa agagtacaaa |
| | agtgaatata tttcctttgg |
| 2221 | aaattttcat tgtgattagt tggataggaa aagatcagag |
| | tttttatcaa gtgatcttta |
| 2281 | aagaattttt tttttttaaa aatggtctcg ctgtgtggcc |
| | caggcttttc tcaaactcct |
| 2341 | gagggcaagc gatcctccca cctcagcctc ctgagtagct |
| | gggactacag gcatgtgcca |
| 2401 | ctagacctgg ctctaaagac atatatgaca cacgaaacca |
| | tttatttttc atttcacaat |
| 2461 | gtttattcac atatatggta ttagtattct aatgtagtga |
| | tgcactctaa atttgcatta |
| 2521 | tatttcctag aacatctgaa cagagcatag gaaattccct |
| | attttgccat tatcagttct |
| 2581 | aacaaaaaac ttaaaagcac tttatcattt catttccctg |
| | cactgtaatt tttttaaatg |
| 2641 | atcaaaaacR gtatcatacc aaggcttact tatattggaa |
| | tactatttta gaaagttgtg |
| 2701 | ggctgggttg tatttataaa tcttgttggt cagatgtctg |
| | caatgagtaa atttagcacc |
| 2761 | attatcagga agctttctca ccaatgacaa cttcattgga |
| | agattttaat gaaagtgtag |
| 2821 | catactctag ggaaaaaata tgaatatttt agcatctatg |
| | tattgaaaat tatgttgaat |
| 2881 | aaatgtcaga ctatttttta cataacgttg cttctgttta |
| | attttgtcac gttcagaggt |
| 2941 | ggggggtagg agatgtaagc ccttgacagc aaaataattc |
| | cttttgcttg atttcagaca |
| 3001 | gttgcatcag ctcctttgtt ctgtgttcat gttacactta |
| | tttaggtggc tgaatccaca |
| 3061 | gaggagcctg ctggttctaa tcggggacag tatcctgagg |
| | attcctcaag tgatggttta |
| 3121 | aggcaaaggg aagttcttcg gaacctttct tcccctggat |
| | gggaaaacat ctcaaggtga |
| 3181 | gtgttataat aaagatcttg gcttatgcaa catgaatgtt |
| | cctcgtttgc atcaatttaa |
| 3241 | gaataaggta tgtttacacg tatataatca gaacttttaa |
| | acatacagaa ttttgcttta |
| 3301 | taaatagctt cgctttaaag atctcttata tatttaactt |
| | ttcttaatac acagcctttt |
| 3361 | agtacacaca aatttaaaaa gtaggtaatg catatattga |
| | aaaaaaaaag aaaatgtagg |
| 3421 | cgttttatcc ttccattgtg ctgaccgctt ggttgccgtc |
| | atgataggaa attagtgctg |
| 3481 | ctgcaggaga aaacagctgt cgtaagcatt gtgcagctgc |
| | tttgctgagt ggctttgtgc |
| 3541 | tttattgtaa caatgggtga caacaaggga gagactgttt |
| | aagaagtgtc cttccaaaga |
| 3601 | cttgggggta ctaggaaaat tggccaattt cttataacta |
| | ttaaagcttt tctttagagc |
| 3661 | aaaagtcaga actaaatgct ctgttatttg gatttttata |
| | gctcttggaa tacattgttt |
| 3721 | ttggggaaaa aftcattaac tgcaagttgc aattctgtaa |
| | ctctccaatt ctccgtcccc |
| 3781 | ctttttctag tactttttat acacaatatt ttataaagcc |
| | aggtgtttta ggaatgagtt |
| 3841 | tttcttcctt ttccccctta atggaacttt caaatataca |
| | caacagtggg gactggataa |
| 3901 | ttaatcccca caccatcacc caggcctaat aaataacaat |
| | cagagtttta ccatagctca |
| 3961 | tctattaccc tttccctttt ttttaaaaaa agagtattta |
| | aaagtaattc ccaggcatca |
| 4021 | tgttatttca tccctatatt cttcagtata taactatgaa |
| | aaccttttag ttatcttgta |
| 4081 | tatctagaat gccattatca gatctaacag aaatcttaaa |
| | agcactttat cattttattt |
| 4141 | ccatgcactg taattttctt aaatgatcaa aaacagcatc |
| | ataccaaagc ttacttgtat |
| 4201 | tggaatacta ttttagaaat actctgtgta tggaatttag |
| | ttaaaagatt gtcagcatat |
| 4261 | aaattggata attattggat agaaaattat tggttagaaa |
| | aacttaaaac tacttccctt |
| 4321 | attctgattc aactattctt aacttgagaa ttgaggctca |
| | tatttgactc tctgaatctc |
| 4381 | accatattta aacttgattt cctttaacaa atatttattg |
| | aacagccagt acatacaact |
| 4441 | ttgcttaagg atatgagttg acctgcagga atgaccacat |
| | aaccaatttc tgatctttgg |
| 4501 | gcaattctat tatggtttca attttgtgat gtgctactga |
| | agaaattgac tgtgtaagaa |
| 4561 | gcacaaagcc aaagatgact cgcatacacc tgccctgtga |
| | agttgagggc tgtgtggcct |
| 4621 | gattaaagaa gcatgcattt ggccgggcgc ggtggctcac |
| | acctgtaatc ccagcacttt |
| 4681 | gggaggccaa ggtgggcgga tcacgaggtc aggagattga |
| | gaccatcctg gctaacacgg |
| 4741 | tgaaaccccg tctgtactaa aagtacaaaa aattagccag |
| | gtgtggtggc gtgtgcccgt |
| 4801 | agtcccagct actggggagg ctgaggcagg agaatggtgt |
| | gaatccggga ggtggagctt |
| 4861 | gcagtgagcc gagattgcgc cattgcactc caggctgggc |
| | aacagagcga gactccatct |
| 4921 | caaaaaaaaa aaaaaagaag catgcatttg acatcagacc |
| | agctctgaaa tccagccctg |
| 4981 | ctacttacta gctcagtgac cttgtgcaaa gcccctgtct |
| | tccctgagct gtacttgggt |
| 5041 | tccccttgta aaatctcctt catgagattg ctgtggaccc |
| | agcagcctgt acaggacccc |
| 5101 | cggtggtcag taagcatgca caggtgatac cacatgcaca |
| | cttcactata gattcagatg |
| 5161 | gaggatggag gaagaagtgg atattgggag gatgggagag |
| | atttccttca ggaaagactg |
| 5221 | ggagaaatgt ggcttttgag ctgctctcta aaagatgaac |
| | agtgggttga gacctgcagt |
| 5281 | gagactcaac aagtcaaaac tgaggtggga tctacttaac |
| | ccaaaaaggt tgtttatata |
| 5341 | tctgtgcact cagcaagcag gtggcaaggg ctgttgtttt |
| | cctgtgggat gggactgtgg |
| 5401 | tagaagtgct gtgtcattta gtaccttctg tgcttgtggt |
| | acatgggtgc gtcatacagt |
| 5461 | catttaccat gtaactgccg taaattccat tcttcYgtct |
| | cttcgatttg aattcttgat |
| 5521 | tttaatttaa acttagtttt catttgttca tactcaaccc |
| | actcaaaatg agtgtttttt |
| 5581 | caaatctatg tataggcctg aagctgccca gcaggcattc |
| | caaggcctgg gtcctggttt |
| 5641 | ctccggttac acaccctatg ggtggcttca gctttcctgg |
| | ttccagcaga tatatgcacg |
| 5701 | acagtactac atgcaatagt gagtccttcc cgccatgctg |
| | ggtgtggcca gggctcccgg |
| 5761 | gaattgaagg gaattttatc cRtgttacct gtaaagttca |
| | gaatggaggg atgagtgtat |
| 5821 | tttcatcagt ctgcagagcc ctgctctgtt tggtctgtgg |
| | gtagagtgta aatgacagct |
| 5881 | gctacctgat ttgtgtattg acacaagRgt gcttttgtct |
| | gatacatagt ttagtgactt |
| 5941 | tatttagaac atgttacttt cttttcaatt tgtgctatct |
| | tcaaagtttt gtgaggattt |
| 6001 | taatttgcta ctgtgccata atcttacagt gggcttgatg |
| | tttaatttca ggtgaaattc |
| 6061 | accttgtggc ttttcttaga aaaggcattt atagatatag |
| | ttagtaagga agttcttcac |
| 6121 | taaattgaag aaaaatcaaa ggcatatcta cctacttgaa |
| | attcttaaca gtaaaagact |
| 6181 | gctgtgtaaa taagccacag acttcacggt gctttggtct |
| | caacagtatc tgcctctgtc |
| 6241 | gtttttattt tagtttagca gccactgctg catcaggggc |
| | ttttgttcca ccaccaagtg |
| 6301 | cacaagagat acctgtggtc tctgcacctg ctccagcccc |
| | tattcacaac cagtttccag |
| 6361 | ctgaaaacca gcctgccaat cagaatgctg ctcctcaagt |
| | ggttgttaat cctggagcca |
| 6421 | atcaaaattt gcggatgaat gcacaaggtg gccctattgt |
| | ggaagaagat gatgaaataa |
| 6481 | atcgagattg gttggattgg acctattcag cagctacatt |
| | ttctgttttt ctcagtatcc |
| 6541 | tctacttcta ctcctccctg agcagattcc tcatggtcat |
| | gggggccacc gttgttatgt |
| 6601 | acctgtaagc agatggtttc tctaatataa attacactac |
| | actgtgttca cactaagcag |
| 6661 | attttgctct ttttgtttct tgtttttttt gagatggagt |
| | ctcgctctgt cattcaggct |
| 6721 | ggagtacaat ggcacaatca ctgcaatctc cgcttcccag |
| | gttcaagcga ttctcctgcc |
| 6781 | tcagcctcct gggtagttgg gattacaggc gcccaccacc |
| | atgcctggct aatttttata |
| 6841 | tttctagtag agacggggtt tggtcatgtt ggccaggtgg |
| | tctcgaactc ctgacctcaa |
| 6901 | gtgatctgcc caccttggcc tcccaaagtg ctggtattac |
| | aggcgtgagc caccactgcg |
| 6961 | cctggccaga ttttgctctt ttttgagcag tctcagttac |
| | tgtagaagga gatgtgttta |
| 7021 | aatagtatat cactctgtgg ctgggcgcag tggctcacac |
| | ctgtaatccc agcagtttgg |
| 7081 | gaggccgagg caggaggatc acatgaggcc aggagtttga |
| | gaccatcctg gccaatatgg |
| 7141 | tgaaaccccg tctctactaa aaatacaaaa aattagccgg |
| | gtgtggtggc acgggcctgt |
| 7201 | aatttacttg ggtggctaag gcaggagaat tgcttgaact |
| | ggggaggcag aggttgcagt |
| 7261 | gagccaagat cgcaccactg cactccagcc tgggtgacag |
| | agcaagaccc tgtcttagaa |
| 7321 | taaatacata ataaatagta tgtattctgg cactttcgat |
| | acaaggaatt catggcttgg |
| 7381 | ttgtatggtc ccaagaacat atcaatcctg tgttaatata |
| | agaatattat cttgtcctct |
| 7441 | agataagcta ccttaccttc caggctcaca aaccacaagt |
| | atgtagccat actgaggcat |
| 7501 | ggaacagaaa actgtatttg ttttgaatga gaacacattt |
| | gcctttatca gRccacagct |
| 7561 | gccttccagg gtaaagtcag gcactgactg ttagcatggc |
| | cctgaaaggc aagggaacct |
| 7621 | tcacatacct gacatttggt tttagctgtg gcccgaagca |
| | gtagttctca actgggggtg |
| 7681 | gttttgtacc ctctctcccc aggggacatt tggcaatgtg |
| | tagatatttt tggttgtcac |
| 7741 | atctagggga agtggtcctg ctggcgtcta gttagtagaa |
| | gcaagagtgc tgctgaattt |
| 7801 | tctacaatgc acaggacaac tcccacagta aatatttggc |
| | tcaatatgtc actagtgcca |
| 7861 | aagctgagaa agcctggcct agagtgatgg gtcttcgtgg |
| | ttgaaactaa aagaagaaat |
| 7921 | tttcctgtat agtaaaaatg gatttttatt ttagctttta |
| | aaaataaaaa aggaataatt |
| 7981 | agaatataat gaacactcaa atatccacaa ttagtcattg |
| | ttaatatttt attatattta |
| 8041 | agacttggga tttaattggt ttcttcaaat aaaacgttta |
| | gttaatattt tctgtagtct |
| 8101 | ctagaatcat atatttaaaa gaccaaatgt aggttgtcat |
| | tatgttatat ataattatag |
| 8161 | ctttgtaggt atatgcattc tccccgctct gttcaggttt |
| | tcaagggtaa ggtttagttc |
| 8221 | gtcataagca tttattgagt gcatactatg tgccaggtgc |
| | tgtttatatg gctgggtgtg |
| 8281 | caatgatgaa taaaacacag tccctgcctt caaggacctt |
| | acagactggt gagaatgtct |
| 8341 | gggagacagt gtgatcaaat gcctctgtag atgggtcttg |
| | cagtgagctt atcagggtgc |
| 8401 | tgctgtgatt agaggtgggg agccttggat tcttgagtaa |
| | gtctcactgt gacatcagct |
| 8461 | aagatggact tttatgtgct tcctttgaag gcatcacgtt |
| | gggtggtttc catttagacc |
| 8521 | gaggccggtt cagaacttcc caaatgatgg tcctcctcct |
| | gacgttgtaa atcaggaccc |
| 8581 | caacaataac ttacaggtat ggagcctccc acgaagccca |
| | ggcgagcttg acgtgatatg |
| 8641 | ccaggctctc caatcctcaa cctttagatt gccagcttgc |
| | ggtttaccat tttattgggc |
| 8701 | taaatatttc atgatttgtt cacatttctt cttagagcag |
| | cagctatttt taaaacgtcg |
| 8761 | aatgtgccat cacattctat cacatatttt tgacgtggca |
| | atttgcattt tggcttaagt |
| 8821 | aaataacatt tttttaaacc cactattttg agcgttcagt |
| | ggtctgtaac agtgtgttat |
| 8881 | accataagaa ctggtatgaa gtggttaact actagtttaa |
| | taatagttga agcctgggcg |
| 8941 | tggtggctca cgcctgtaat cccagcgctt tgggaggctg |
| | aggcaggtag atcacctgag |
| 9001 | gtcaggagtt taagaccagc ctggccaaca tggtgacacc |
| | ccgtctctac taaaattaca |
| 9061 | aaaattagtt gggcgtgcct gtattctcag ctgctaggga |
| | ggctgaggcg gaaggatggc |
| 9121 | ttgaccttgg gaggtggagg ttgcagtaag ccaagatcac |
| | accactgcac tccagcctgg |
| 9181 | gtgacagagc Ragactctgt ctcaaaaata ataataatag |
| | ttgcagatct agttgtttca |
| 9241 | tttgatattt gctgccagga gcagtcaaaa ctatgacaac |
| | atcaacacag ttgtgctgtg |
| 9301 | gaagcctgag aaacagccct atccagccta gggcatcatt |
| | ccctggggtc ctggctgcct |
| 9361 | gctgctgtgc ctatggggca gggggcaggg atttaccctg |
| | ccctttccta acattatttg |
| 9421 | gtgttcatca tagccctaat tgttttctca ttgtttcatt |
| | acctcattgt ttcattacct |
| 9481 | gtaggaaggc actgatcctg aaactgaaga ccccaaccac |
| | ctccctccag acagggatgt |
| 9541 | actagatggc gagcagacca gcccctcctt tatgagcaca |
| | gcatggcttg tcttcaagac |
| 9601 | tttctttgcc tctcttcttc cagaaggccc cccagccatc |
| | gcaaactgat ggtgtttgtg |
| 9661 | ctgtagctgt tggaggcttt gacaggaatg gactggatca |
| | cctgactcca gctagattgc |
| 9721 | ctctcctgga catggcaatg atgagttttt aaaaaacagt |
| | gtggatgatg atatgctttt |
| 9781 | gtgagcaagc aaaagcagaa acgtgaagcc gtgatacaaa |
| | ttggtgaaca aaaaatgccc |
| 9841 | aaggcttctc atgtctttat tctgaagagc tttaatatat |
| | actctatgta gtttaataag |
| 9901 | cactgtacgt agaaggcctt aggtgttgca tgtctatgct |
| | tgaggaactt ttccaaatgt |
| 9961 | gtgtgtctgc atgtgtgttt gtacatagaa gtcatagatg |
| | cagaagtggt tctgctggta |
| 10021 | cgatttgatt cctgttggaa tgtttaaatt acactaagtg |
| | tactacttta tataatcaat |
| 10081 | gaaattgcta gacatgtttt agcaggactt ttctaggaaa |
| | gacttaYgta taattgcttt |
| 10141 | ttaaaatgca gtgctttact ttaaactaag gggaactttg |
| | cggaggtgaa aacctttgct |
| 10201 | gggttttctg ttcaataaag ttttactatg aatgaccctg |
| | gcagagactc ctgtcatcct |
| 10261 | agcagtttac tctgcgtttg ttgtatctag acagtcaaca |
| | actgagttgt cggtgtttaa |
| 10321 | cctgaatgct tgtttttcag aagaRgactg tttgtgccgg |
| | taagaatgat caggtaaggc |
| 10381 | catgaaagtt tttgttggyg tttttgtttt tgagatgggg |
| | tcttgctttg ttccttgggc |
| 10441 | cagagtacat tggctactca caagtgggct ggtagctggc |
| | tacagcccca gactcctggc |
| 10501 | ttaagccacc tcctgcctca gccaccctgg cagctgggac |
| | tacaggcatg cgccatcaca |
| 10561 | cttagcttga aagttttaat ttactaagaa tatacctgtg |
| | tttcccccca tttcctgatt |
| 10621 | taaacagtac tggcttatat aggaacccat caaagttaaa |
| | ttccccaaat ttaaatttag |
| 10681 | taaatttagt ggtttcacct tggcaaatct gcaatagttt |
| | caccagctca aatttcatgc |
| 10741 | ttttgtaagc tgagcttatg tttgtgattt taatccttta |
| | agtactactg tggtaactaa |
| 10801 | tcattttttt gttttttttg gtggtgtttt tgtttttgtt |
| | ttgagatgga gtctcactct |
| 10861 | gttgcccagg ctggagtgcc gtggcgcgat ctcggctcac |
| | tgcaacctct acctcctgag |
| 10921 | ttcaagcgat tctcctgcct cagcctcccg aatagctggg |
| | attacaggtg cccaccacaa |
| 10981 | tgtctggcta attttttgta tttttagtag agacgaggtt |
| | tcacccatgt tggccaggct |
| 11041 | ggtctcaaac ccctgacctc aggtgatctg cccgcctcgg |
| | cctcccaaag tgctgggatt |
| 11101 | acagatgtga gccaccgcac ccagccaggg actaatcttt |
| | aaagcaaagt tttatatatt |
| 11161 | tttacgtgag taatgttatg tgtaggtgtt ctatttggca |
| | aaataaatca gccttttcta |
| 11221 | tcatgattgt ggtcatttaa attaatcctc atcggaaaca |
| | ttgtttacct aagataaaca |
| 11281 | ttcactaaac aactcatagc aagacgctgg gtaggtacaa |
| | agatgtctta tgtgtggtta |
| 11341 | tgctttcaaa gccaactgaa acccttttgt aaggagttaa |
| | atagcaaaaa gtttcaaata |
| 11401 | actgtgtgcc tctagaacag aactatcaga aggcaatgaa |
| | tattcacaaa ttgtgaagag |
| 11461 | atagtttgct tcaaggaaga aatgacttgt aactagagta |
| | ggtatgaaat gatgggggaa |
| 11521 | aaatgatgag gtatgaaatg actcaccttc tctccagctc |
| | aaggtaagaa gggtggcagc |
| 11581 | aggagtaaaa gctcagccac aggacgtgct cttcctgcca |
| | aacagctgga ttctgcacat |
| 11641 | gcttgggaat gaggtcctgc tggtttagga aaacaaacat |
| | taagaattcc caaagtcttg |
| 11701 | ggtggaaaag cctgttgctt ttcagaggca aggccatcac |
| | catttggcaa ggggcccaag |
| 11761 | gccccaggca gctgtggtac catctgtttc tgaaggagtg |
| | gggattttac cccctgaaat |
| 11821 | gtcagtttgt ggcttaaact ctgggttcta caggcccaaa |
| | ataaatagtg ccaggaaggt |
| 11881 | gcccctgaac agagctaacc agggttcaga ttggcacccc |
| | atccccactg gggcaagcag |
| 11941 | gactcttagc aattaaggcc taagacggct gacaggtggg |
| | acatggcaga aattcagtgc |
| 12001 | ttccaccaca gctactcaat gtgccacRat catgggccac |
| | acaccactct gaatacttag |
| 12061 | aaaattattg gtttgggcct ttattattaa gtagcaagta |
| | aataactgaa aaagccccag |
| 12121 | atttcagctc tggctatagc agctttctaa gtctgtgttt |
| | ttaaggtgca atttcttgaa |
| 12181 | tcctagttct gtagaagaaa cactctaccc ccccgccccc |
| | cccacccccc ccccccaccc |
| 12241 | ctaaaaaatg agcttcatga tcaagtacat tttggaaccc |
| | tcaggctgat ccaagttcat |
| 12301 | agatttcttc actgcaggcc ttctgggtcc tttaatatgc |
| | ctgaagctgg ggctttccct |
| 12361 | tcctgagaat ggcccaggac tatccatgtg cctcattttg |
| | ggaaatttag taatgagtta |
| 12421 | ctaaatcaac accacctttg gcattctaca tagcaaacat |
| | actttgttag atgtgacatt |
| 12481 | ttacacacta accaatggag ataaaatttt ttaaatttat |
| | ttttggccag gcacggtggc |
| 12541 | tcatgcctgt aataccagca ctttgggagg ccgaggcagg |
| | cggatcactt gaggtcaaga |
| 12601 | gtttgagacc agcctggcca acgtggcgaa atcctttctc |
| | tactaaaaat acaaaaatta |
| 12661 | gctgggcgtc gtcgtgggca cctgtaatcc cagctactcg |
| | ggaggctgag ggaggagaat |
| 12721 | tgcttgaacc cgggaggggg aggttgcagt gagccaagac |
| | cacaccactg cactccagcc |
| 12781 | tgggcgtcag agtgagattc tgtctcaaaa aataaataaa |
| | tgatttctta tttatttttt |
| 12841 | tttaccagtg ctctacatgt tcagagaaac ttctctggta |
| | accaaccaca gaaatgatcc |
| 12901 | ctgaaagtat agtcttagga gataaatatt ctttacatag |
| | ccaggaggga gatgagaagc |
| 12961 | catacctgtc tacagtctga ctgacaaagg aggaggctcc |
| | aggtcccctg gagaatccca |
| 13021 | ggttttatgt aataattact tggtcctttt caaagtactt |
| | ggatgagtgt aggatagtct |
| 13081 | ctttgtctta tcagctagtc ctgcaatcta ggtgttctcc |
| | cattttgtag aggcttgcag |
| 13141 | agatgagaga cctgtccaag gtctcaaacc cctaactggc |
| | agttctgaga ctagaaatac |
| 13201 | tccctctcag gagtggacct cctctacaat tttttttagc |
| | agtcctcatg tgccaaatat |
| 13261 | ggcgtggggg ccaggagaca gaatgaggca taggttgggc |
| | gtggcccctc ccctcttggg |
| 13321 | tcttgtagtc ggctgtgggt ggcaagtaca gcagYgatgg |
| | cagacaggtg tgatgagttc |
| 13381 | aaggacaggg aaataacatc cacagcccca tcccagtact |
| | cctgtgcagc aatggaagtg |
| 13441 | tgccaccgac acaggggcta cagggtgagc aggagtgaca |
| | ggcgaagaga ggaagagagc |
| 13501 | tctgcctgtg caaaggcgga gggaacatgg tgtgggagga |
| | actgaagaga attccacata |
| 13561 | agtatgaaga gggtagggag attgagactg gagaaaaggg |
| | gaagccgggg ctcccctttt |
| 13621 | gaccatgcaa cgcactggaa gcacagcact cccagcctca |
| | gggtcctcca tgattggaga |
| 13681 | ctgacaRtga attcagagga gcttgtggaa gtcacccagt |
| | caggtccaga aatcctgcag |
| 13741 | tgcatccctt ccgttacccc tgtcataact gggagaagac |
| | accacccaca tgacgcaggg |
| 13801 | gagagtgcay gcatgagcta gcacccatat tgtcccctgc |
| | atctgtcctt ggctatgttt |
| 13861 | tacacccccc accccttaga acattgtagt caaactagga |
| | tgtattaaat tggctggttg |
| 13921 | ctcccaacta cactggacaa agtggagaaa aaaaaaaaag |
| | gaggagctca aggtttcaaa |
| 13981 | ttcccagctc aagctctgca taaaaactgc tgtgacttcc |
| | ttagaagaga cccctatctc |
| 14041 | ctgtagctgc agggttgaga tttctgagct caagtgatct |
| | gcctgcctca ccctcccaaa |
| 14101 | gtgctgggat tacagttgtg agccaccctg cctggccagg |
| | gctgagattt gtgaaaagga |
| 14161 | aacccaaagt ctcatcttgc atgtggctga attatgtcgc |
| | aaatccaatt cccaaattcc |
| 14221 | agccttgcag ggtgtcttca gttaaagtga ggacaacttg |
| | aaaatcggaa tgggaacata |
| 14281 | agggaagatt ccagtaaagc tggggacatt gaatccctaa |
| | attctgtYat gttccctgcc |
| 14341 | agtagaaaca gcccatctac ccctccctgg gtccagctgt |
| | gtggacacag attggcagcg |
| 14401 | agtgtgcagg tgttatttcc ctacccttgt aatcatcaca |
| | ctttcttttt ctttttcttt |
| 14461 | cttatttttt tgagtcagag tcttgtgctg ctgcccaggc |
| | tggagtacag tggtgcaatc |
| 14521 | tcagctcact gcaacctccg cctcctgggt tcaagcaatt |
| | ctcctgcctc agcctcccga |
| 14581 | gtagctggga ctacaggcgc gcaccaccac acccagctaa |
| | tttttttgta tttttagtag |
| 14641 | agacagggtt tcacccccaa tagccaggtt ggtctcgaac |
| | tcctgacctc aagagattca |
| 14701 | cccacctcgg cctcccaaag tgctgggatt acaggcgtaa |
| | gccacYgtgc ctgacctcat |
| 14761 | cacaatttct tagccctttg agaaaccctg tggccttccc |
| | tgaggtagct gtcttgcggg |
| 14821 | gcacttctga ttcttcttgg atcctatgcc cttcccctct |
| | ttgctggacc tgtaactaga |
| 14881 | ctcaagtccc accaggcccc agagagaaag gtacacactg |
| | tgacccatga agaggtgtgt |
| 14941 | tacacaccaa aagaactgca tgatttttcc aatttataca |
| | gacaaatcca agagaYatgt |
| 15001 | atgggagtag attttaaggt tgtgagatca cggaaggacc |
| | atagtgttgg atctggctga |
| 15061 | acgtaatgat atgggcccac taagcagaga ttcttaactc |
| | agtgctttag catgaggggt |
| 15121 | tagaaagggc tctaacttgg ttggttggtt gtttggttcg |
| | ttggttggtt ggctggctga |
| 15181 | aacatggacc aaaaggtagg tggccgacac cgttccttat |
| | tttgttatta atatatttgt |
| 15241 | atattcatat acaaacttgt atgctatgat atctaataca |
| | tattacagat ataaaacaaa |
| 15301 | tctttaggcc aggcatggtg gctcactcct gtaatcccag |
| | cacttcggga ggctgaagca |
| 15361 | ggtggatcac gaggtcagga gttcaagacc agcctggcca |
| | acagagtgaa atcccatatc |
| 15421 | tactaaaaat acaaaaatta gccggtcacg gtgtcaggca |
| | cctgtaatcc cagctactca |
| 15481 | agaggctgag gcaggagaat tgcttgaacc cggaaggtgg |
| | aggttgcagt gagctgagac |
| 15541 | cccatcactg cactccagcc tgggtgacag agcaagactc |
| | cgtctcaaaa aaaaaaaaaa |
| 15601 | tcttttgttt ctttcctctc tcattccttt atcacctaac |
| | ataagatgga tcatcacagc |
| 15661 | attgaagtga caggatgtca agcagaagag taaacatcac |
| | ccaaggacgt tgcagcctct |
| 15721 | ctggggaaag gattggcatg ttttctgttg tatgcaggat |
| | ggttgagtca tgttaggtgg |
| 15781 | aagtatggct ttgttactgt ctttatctgg agattaagtg |
| | tggtttaagg agatgcatac |
| 15841 | aggtgccagg ttgacaaaga gagtactgta atgattagtt |
| | ttatgtgtga acttggctag |
| 15901 | gcccagttat gtaaacacga atctaggccg ggctttggtg |
| | gctcacgcct gtaatcccag |
| 15961 | caatttggga ggctgaggca ggcggatcac ctgaggtcag |
| | gagtttgaga ccagcctggc |
| 16021 | aaacatggtg aaaccccatc tctaccaaaa atacaaaaat |
| | tagcctggcg tggtggtgtg |
| 16081 | tgcctgtagt ctcagctact cgggaggctg aggcaggaga |
| | atcgcttgaa cctgggaggt |
| 16141 | ggatgctgca gtgagtggag atcatgccac tgcactccag |
| | cctgggcaac agagtgagac |
| 16201 | tgtatcgcaa aaaaacataa aagaaaaaac aacaaaaacc |
| | ccgtgaatct aggtgttact |
| 16261 | atgaaggtat tttgcagatg tgattaacct gcatgggcct |
| | catctaatta tctgaaagac |
| 16321 | cttaattgca aaactcaggg ttccccaagg acagagcaat |
| | taagtctatg gcctgcagcc |
| 16381 | tcagcttccg cctgagtttc cagcccacca tcctacccta |
| | cagcttcaga cgcgtctagc |
| 16441 | cagcccctac aactgtgtag gacaatttct agaagttagt |
| | taatgaatac gctattggat |
| 16501 | ctgtttctct ggaggaaccc tgactgacac aggggccggc |
| | tctggaggtt gtggggtcca |
| 16561 | gttccttatc cctgtgtagt gtattggcca gttggggtgg |
| | gaattgcttc caggcaggct |
| 16621 | gtgtcatttg ttcaggcagg gtcccctccc cttggctctg |
| | cctgagagca aaggcaggtg |
| 16681 | ggggtggccc tggggagggc ctgaagcttt gctctgtgcc |
| | tcctaaagct ttcccactcc |
| 16741 | agggaccagg ccaggcaggc tggcaggaag aggcaggctt |
| | catctggaag gatgtctcca |
| 16801 | gggtggaaat gatgagattt aaatggtgta gggacccgga |
| | gggtggggtg ggacagcacc |
| 16861 | actgacccag ctgcagagtt ggaccctctg aagcccattg |
| | tgtacatgag aatctgtgtg |
| 16921 | cctgattgcg taggtgccta tacatggata ggctgcgaat |
| | ctgtgtgcac acagctggct |
| 16981 | ggagagcaca tgagtaggta tatgtaccac attgtcagca |
| | ctcacccatt agctcctggg |
| 17041 | gactcacttg ctgcaggtga tccctgtcct cgtgggctct |
| | acaccctggc aggtcaacac |
| 17101 | agactacaaa gaaacaagcg gacataaaat gacaagggct |
| | aggacaaaaa gcatggaaag |
| 17161 | gagaggggat aacgtggcag gggacatttt agaatgcagt |
| | ggtttcgaca ccaggaggag |
| 17221 | gagtggttag acagtggtcg gggagggcct ctgaagaggt |
| | gacacctgaa tgatgagatg |
| 17281 | gagcaaacca agcaaagatt ggaggaagag cctccaagca |
| | gaaagaactg caaggacaaa |
| 17341 | ggccctgggg caggagcggg tacgtgtgtc ctgtatgtgt |
| | gtgtcctgca cggtggcagg |
| 17401 | tgtctgaagc tgagcccaga ggagctgtgg agatcaggcc |
| | cagaagtgga gccacagcac |
| 17461 | gtagggccag agacctgcag taagaaatgg gatgtggtcg |
| | agatgggatg ggagccaccg |
| 17521 | cagcaccatg gggcgggagc agcagagtct gatgttgtgt |
| | acttcaggga gctggagttc |
| 17581 | tatgaaggaa gagcgaggag gcatgtggga ggaagaacag |
| | ccccactgag gcctgcaggg |
| 17641 | aaggcagcag accccaggtc agagcatgaa ggtagagggg |
| | aggttccaag aagtgggttg |
| 17701 | gggaataagc actggatggc tgatgacaga gggcagggtg |
| | gaggatgcgg ggccagggag |
| 17761 | ggctgcacag agtcctggga gggtgagcgg tggccctggc |
| | tggctaggag gcgccacctt |
| 17821 | gaggtgctac tggaggatac gccgcaggga aggtggatgg |
| | tctcttctga tgacaacctt |
| 17881 | cttgctggca ttagactgaa aggggaggtg atttgtaggg |
| | atgaggccag aggggttgtc |
| 17941 | cccagggaag ggcaccagat gtggggcagg agagctggtg |
| | tgagttgcag ccgtggggcc |
| 18001 | tagaggaagt cacagcaccc cactgaattc tcacttcctc |
| | atcataacag accctgtgac |
| 18061 | atataaaacc tgctctgccc accgcacagg gatcacaagg |
| | ctggagtgaa ataatggagc |
| 18121 | acttccccag agtggcacac gggacccagg gacgggtgtg |
| | caaaaccgtt ctaggtggaa |
| 18181 | gaacactttt tttttctttt tctttttctt tcttttcctt |
| | tttttttttt ttttttttga |
| 18241 | gatggagtct ccgtctgttg cccaggctgg agtgcagtgg |
| | tgcaatcttg gcccactgta |
| 18301 | gtccctggct cccaggttca agtaattctc ctgcctcagc |
| | cttctgagta gctgggacta |
| 18361 | caggcaccca ccacaacatc tagctaattt ttgtattttt |
| | agtagagaca gggttttacc |
| 18421 | atgttgacca gactggtctt gaactcctga cgtcaaggga |
| | tccatctgcc ttggcctacc |
| 18481 | aaagtagctg ggattacaag catgagccac cgtgcaaagc |
| | caaaaacact tgattataat |
| 18541 | agcatgctat ttttcaatgt attagataaa tgtattttcc |
| | atttaaagtg gggatataaa |
| 18601 | gctccccttt aaaatccaca taagtaaaaa agtcagtctg |
| | tttaaaaggt taagtaaata |
| 18661 | acagtctagg gcagatgcac aaaagacagc atcatgaccg |
| | tggttcctga aggattaagg |
| 18721 | ttgcaggaaa cactgaatgc taaaagcttt gtgaatgtat |
| | agtccatgaa aatgtctttt |
| 18781 | aaaaaatgag gtgcatacca agatgtttga gtgtgtgtat |
| | ttggggtagg tgctgtgggc |
| 18841 | atacgggtgt acctgtgtgt gtctcacaag caggtgtagc |
| | tgggtgcttg tgtgtatgtg |
| 18901 | gtgtgtgtgt atctgtgcac acgtgcatat ctgtgcccac |
| | atatccatgc acacgtcctc |
| 18961 | ataaggctct ctgcggccag gtgcggtggc tcacgcctgt |
| | aatcccaaca ctttgggagg |
| 19021 | ccgaggcggg gggatcacga agtctgaagt tcgagaccag |
| | cctggccaag atggtgaaac |
| 19081 | cccatctcta ctgaaaatac aaaaaattaa gacagtgagt |
| | gttgatcgtg aagaagggaa |
| 19141 | gatgaatgtg gggattaggt gggaggaatg gggtgctgtc |
| | aagatgaaaa taaacttagg |
| 19201 | gtggagaggc tggggcaaaa gagaggccca acagtatgat |
| | ttgccccagt gcggggactt |
| 19261 | tgtgcctggt gatttgaggt ggcagggctt ctacccaggc |
| | ctgatgggag ctgggattaa |
| 19321 | aagcagctgt gacctaaggc aggccattgc tctgtggcct |
| | Rctcccctaa ctcttttgca |
| 19381 | gaaagtccca ggctaagaga tgggggatgg ttgtaatgac |
| | ccctcccaga aatcctggct |
| 19441 | ctgtcctgca gaggtggggc ccggggatca agacgcctct |
| | aggttcaaga gagaggcaga |
| 19501 | atgaggggtc agccagctgg ggcaggaagg cttcactcta |
| | ggctgcaagg agagaccttt |
| 19561 | cgccttgact ggaaggacgt gaggagcctg tggcctcctg |
| | tttgcaacca agggctgagg |
| 19621 | aaggggccgg gcattcctgc tgtaggtcaa tgacctggag |
| | aaacacatgc ttgaggacag |
| 19681 | agctcagatc attgtcccag cgcaaacctt ggtgggatgt |
| | aaggtttgga gcttcttaag |
| 19741 | ttccaagagg cagtctcatg cagttcaagg agggctggag |
| | aagcctggtc tgctcatggg |
| 19801 | agtcaattgt taaattttca ggaattttgt gagccaatta |
| | ttgaacacag ctattattaa |
| 19861 | aaacaaaatt ataggctggg cgtgatggct catgtctgta |
| | atcccagcac tttgggaggc |
| 19921 | caaggcggtg gatcacttga ggtcaggagt tcaagaccag |
| | cctagccaac atagcaaaat |
| 19981 | cctgtctcta ctaaaaatac aaaaacatta gtcgggtgtg |
| | gtggctcaca tctgtaactc |
| 20041 | cagctacttg tgaggctgag gcatgagaat tgcttgagcc |
| | caggaggcag aggttgcaat |
| 20101 | gggctgagac cacgccattg cactccagcc tgggcaacag |
| | agggagattg tgtctttcaa |
| 20161 | aataataaaa taaaataaaa taaaataata ggctgggtgc |
| | cgtgactcac gcttgtaatc |
| 20221 | tcagcacttt gggagaccaa ggctggaaga tcgcttgagc |
| | ccagaagttg gagatcagcc |
| 20281 | tggggaacac agggagaccc tggtctgtac aaaaaataaa |
| | aacaaaatta gccaggcatg |
| 20341 | gtggcacatc cctgtggtcc cagttactta ggaggctgag |
| | gtgaacaaag ttaacactca |
| 20401 | aaactcatca cttgtgaatt actttgctac agtgcactat |
| | tattattatt tatttatttt |
| 20461 | tactgggcag cctcttgagc ctaagtaggc ccagagactg |
| | tccagcatac tattattgat |
| 20521 | gctttgtggt tattgatatt gattggatcc gtatagtgga |
| | aatggctata ctgtagaatt |
| 20581 | gtgtgctact gtgtatttct taccaattct gcattcagtg |
| | gcatcacatt catagcttga |
| 20641 | agttggccat ggtgggaggt aaatatgtgt ggtataaatt |
| | ggcaaagact acagattggg |
| 20701 | gttcctgtct ggccccccaa gagctggttg ttaaactttt |
| | accagcacac cactgagttg |
| 20761 | gagtctggaa agcaggcttc gtccaggttc tgccctgcta |
| | gctcaactgg ctctcaaagc |
| 20821 | tttgctccac caactggcct atggctgctt ccactcacca |
| | attctccctt ttgccctcta |
| 20881 | aggccacctc ttccaggaag tttcccctga tttctttgcc |
| | tggacttcta aaattctttc |
| 20941 | tttgccaggc gtggtggctc acacctgtaa tcccagcact |
| | ttgggaggac gaggtgggca |
| 21001 | gatcacttga ggtcaggaga tagagaccag cctggccaac |
| | acggcgaaac cccgtctcta |
| 21061 | ctaaaaatac aaagattagc tggacatggt gacgcgcacc |
| | tgtaatccca gctactcagg |
| 21121 | tggctgaggc aggagaatca cttgaacccg ggaggcagag |
| | gttgcagtga gccaagatca |
| 21181 | caccattgca ctccagcctg ggcaacagag ggagactcca |
| | tctcaaaata aataaataat |
| 21241 | taattctttc ttacctcttc cgttcatatt catacagaat |
| | aatctccaca tctcgaaatt |
| 21301 | cttaactgat tagctagttc ttgctattaa ttaaatacgt |
| | gatgcatatg gccaagcttg |
| 21361 | gatggatgga atccctgtat tggtaaggtg ggaatttttc |
| | ttttcttttt tttttttttg |
| 21421 | agaccaagtc tcactctgtc acccatgctg gagtgcaatg |
| | gtgtgatctt ggctcaccac |
| 21481 | aacctctgcc tcccaggttc aagccattct cctgcctcag |
| | ccttcctagt agctgggatt |
| 21541 | acaggcacac accaccaagc ccagctaatt tttgtatttt |
| | tagtagtgac agggttttgc |
| 21601 | catgttcgcc aggctggtct ggaactcctg gcctcaagtg |
| | atctgcccac ctcagcctcc |
| 21661 | caaagtgctg ggatcacagg tgtgagccac cacgcccacc |
| | tagggtggga attttccacc |
| 21721 | tgggttctgg gctcagctct gccacagatt cctctctctg |
| | ggcttatcct ctgaRggcga |
| 21781 | gattttggca tgactgagtg gatggaggaa gaacttgggg |
| | tcagcgggct gtcaccctgc |
| 21841 | tctgccgtgg aaccgactca acaacagggc cacaacaggc |
| | cttgttggac atggaagcaa |
| 21901 | atgagaaaat tagtaatact gattcagtgt ttacttaagg |
| | ttctgatact ttgtccatta |
| 21961 | tagacctttt gcattaattt tgatttttta ttatataaaa |
| | tatatatata acataaaatt |
| 22021 | gatcatttta gccattcgaa gtgtacagtt ttgtggcgtt |
| | taatacattc acattattat |
| 22081 | acaacccatc tccagaactc ttttcacctt gaaaaactga |
| | aactctattt ccattaaata |
| 22141 | tgaactcact ccccgttctc tcttcctcca gcctctggtg |
| | acctccattc tattattatt |
| 22201 | attattatta ttattattag agagagtgtc cctctgttgt |
| | ccatgctgga gtgcagtgat |
| 22261 | gcaatcacag ctcactgtag cctcaaactc ctgggctcaa |
| | gggatcttcc cacttcagcc |
| 22321 | tcctgagtag ctgagattat aggcatacgc caccacactc |
| | tgctaattca cttatttttt |
| 22381 | ggaaagatgg ggtcttgctg catggtccaa gccagactta |
| | cattcctggg ctcaagcgat |
| 22441 | cctcccacct caacctccca aaacattgag attacaggtc |
| | tgagccaccg caccctgccc |
| 22501 | attctacttc ctggctctta aatatgacta ctctagatac |
| | ctcatataag tggaatcata |
| 22561 | cagtatttgt ctttgtaact ggttcatttt acttggcata |
| | atgtcctgaa ggttcatcct |
| 22621 | catcgtagtg tgtgtcagaa tgcccttcct tttaaaggat |
| | gaataatatt cccttgtgtg |
| 22681 | gatatatcac atgttgttta tccactcatc attgatggat |
| | gcctgtgttg cttccacttt |
| 22741 | tggctgctat ggaaaatgct gctatgaata cgggtgtgca |
| | aatattcatt tgagtccata |
| 22801 | attttgattg ttttttcttt ttgagacaga gtctcacact |
| | gctgtccagg ctacagttca |
| 22861 | gtggcgcaat cttggctcat tgcaacctcc acttcccggg |
| | ttgaagtcat tctcctgcct |
| 22921 | tagcctcccg agtagctggg attacaggta tccgccatca |
| | cacctggcca atttttgtat |
| 22981 | ttttagtaga gacagggttt caccatgttg gagactggtc |
| | tcaaattcct ggcctcaaat |
| 23041 | gatccaccca cctcggtctc ccaaagtgct gggattacag |
| | tgagccacca tgccgagtcc |
| 23101 | ataattttga tttttaaatg gtggctacta agtttttctg |
| | gcacccctta aattttgtca |
| 23161 | ggcacttgcc ttaccctagt cctggcccca gtttgggttg |
| | tcacagcaag gtggaagtgt |
| 23221 | actgaaaagc tcgtgtttta tctttatacR tttacatggg |
| | gccccaaaaa gggggaaaac |
| 23281 | ccactttgat agggatatga cacagggacg caggagccaa |
| | ccgaaagagc ccccagtggc |
| 23341 | caaagatsga gcaacttgag caacaaaata aataaagtaa |
| | gcatcaaatt atagcccaaa |
| 23401 | gtataaaata agtgtctatg agtctatact cacatagatg |
| | atttaataca ttcatattaa |
| 23461 | taaatgggag agcactttga gaggccaagg tgggcagatc |
| | acttgagccc aggagtctga |
| 23521 | gaccagatta ggcactgtga tgaaaccctg tctctaccaa |
| | aaacacaaaa attagctggg |
| 23581 | catggtgggg catgcctata gttccagtta cttgggaggc |
| | tggggaggga ggattgcttg |
| 23641 | agcctgggag acggaggttg cagtgagctg tgatctcacc |
| | actgcactcc agcctgggca |
| 23701 | acagagtgag gccctgtctc aaaataaata aatatataaa |
| | taagataaat atataaataa |
| 23761 | aaataaaaat aaacgggaaa gaagagacaa atcttctgtg |
| | caggagaatY ccaaatgaat |
| 23821 | tctgtagatt ctccacccta caggagggca cacacaactc |
| | caggctgcct tagtgacctt |
| 23881 | cttccaaaga gtacagtacg ggaaggggga gcggggagaa |
| | ttaacttcac agtggagaaa |
| 23941 | tctgacaaat acgacctccg ccaagtgatc gaggtcacat |
| | cagctgtcat gaattatgtt |
| 24001 | gatagtgggc atcctggata tgatgggatg aaatggcact |
| | ttacctccac gacccttttc |
| 24061 | acaacagccc gtaatgccag tataaggaaa acatcagacc |
| | aggagtggtg gctcacgctt |
| 24121 | gtaatccgag cactttggga ggccaaggtg ggtggatcac |
| | aggtcaggag tttgagacca |
| 24181 | gcctggacaa catggtgaaa tcctgtctct actaaaaata |
| | cacaaactag ccaggcacgg |
| 24241 | tggcgggtgc ctgtaatccc agctacttgg gaggctgagg |
| | cagaagaacc gcttgaaccc |
| 24301 | gggaggcgga ggttgcagtg agctgagatc gcgccactgc |
| | actccagcca ggttgacaga |
| 24361 | gtgagactcc atctcaaaaa aaaaaaaaaa aaaagaaaag |
| | aaaagaaaaa gagaaaagaa |
| 24421 | aaacatcaga cagatcccaa cagaagggca tcctacagta |
| | tacRtgacca ctgctcctca |
| 24481 | aacctgtcaa gactatcaga aacaagagaa actgtcacag |
| | ctacaaggag atgtgacaat |
| 24541 | taattgtgat tttttttttt gagatgtggt ctcgctttgt |
| | cacccaggct gcagtacagt |
| 24601 | ggcacaatca cagctcactg cagcctcgaa ctctggggct |
| | caagtgatcc tcccatccat |
| 24661 | ctcagcctcc caagtagctg gggctatatg cgtgcaccac |
| | cacacccagc taatttttgt |
| 24721 | atttttttag agacggtatt tcgccatgtt gcccaggctg |
| | gtctcgaact cctggattca |
| 24781 | agcaatccac ctaacttggc ctcccaaagt gctgggatta |
| | caggcatgag ccaccatgcc |
| 24841 | ttgccaaatg tgatgtattc ttgatgggat cctggaagag |
| | gaaaaagata ttaggtaaaa |
| 24901 | actaaggaca tctgaataac catggatttc agtaatgtat |
| | ccacactgat tcattaattg |
| 24961 | taacaaatat accgtatgaa tgcaagatgt tcataatgag |
| | gccggggcgg tggctcatgc |
| 25021 | ctgtaattcc agcactttgg gaggctgagg cggaaggatc |
| | tcttgagctc aggagttcga |
| 25081 | gaccagcctg ggtaacacag tgagatcccc atctctacaa |
| | aaaatttttt gaaattagcc |
| 25141 | aggtgtggtg gtggcacatg cctgtagccc cagctactca |
| | gaagactgag acaggaggat |
| 25201 | tgcttgagcc gggaggttga ggctgcagta agccaggatc |
| | acgccactac actgcagtct |
| 25261 | gagcaacaga gcgacaccct gtctcaatga taataataac |
| | aataataata ataatggaaa |
| 25321 | ctcagtatgg gttgtatatg ggaaacttgc tcagtttctc |
| | tgaaaatcta aaattcttct |
| 25381 | gaaaatcaaa gtctgcttaa aaattcacat ggggtttgca |
| | ttctcctcca tgacaaggtt |
| 25441 | ttgcaggtta tgattacaga ggctggagag ggtgcaggtt |
| | atcccgcccc tccctgtccc |
| 25501 | agggattccc ttccccagga gctgtgtctc ccctgtgaga |
| | gagggtgagc ttccatgacc |
| 25561 | ccaagcctct tgccctctga ctccggtatt cttagaagct |
| | gggaccagca ctgagcccaa |
| 25621 | attcccgaag cgctcaaata ctggctttct gtccctatgt |
| | gacctggagc ttgtagttta |
| 25681 | acttctctct gcctcactgt ttgacctata aagcagggca |
| | atcaaggcat cccgggggtg |
| 25741 | gctatgaaga gtgaatgaga tagcagacaa cccagatgcc |
| | taccgacagg tgaagggacc |
| 25801 | aacacagtgc ggtataggcg tataagggaa tggagtatgg |
| | acacagccta caacacagac |
| 25861 | aaaccttgaa gacattcctc taagggacat aggccaggca |
| | tggtggctca cacctgtaat |
| 25921 | cctagcattc tgggaggccg aggcgggcag atcacttgag |
| | gtcaggagtt gagaccagcc |
| 25981 | tggccaacat ggcaaaatcc cgcctctact aaaaatacaa |
| | acattagctg ggtgtggtgg |
| 26041 | taggtacctg taatcctgga tactcgggag actgaggcaa |
| | gagagttgct ggaacccggg |
| 26101 | aggtggaggt tgcagtgagc cgagattgtg ccactgcact |
| | tcagcctggg cgacagagcg |
| 26161 | agactctgtc gaaagaaaga aagggaggaa ggaaggaaga |
| | aagaaaggga agggaaggga |
| 26221 | agggagggga ggggagggga ggggagggaa gggagagaga |
| | aagaagaaag agaaagaaag |
| 26281 | gaagaaagaa aaagaaggaa agaaagaaag aaagaaagaa |
| | agaaagaaag aaagaaagaa |
| 26341 | agaaagaaag aaagaaagaa agaaaagagt gaacccagga |
| | acgaaagatc acacacactg |
| 26401 | tatgactcta tttacatgaa atgttcagag taggcaaatc |
| | catagagaca gaaagcacat |
| 26461 | ttatggttgc caggagctgg gaaagggcag gatggggaat |
| | gactgtttat tggatgtggg |
| 26521 | gctctatttt ggggtgatga gaatgttctg gaattaaatt |
| | catggctgca taacactgtg |
| 26581 | aacatactaa atgcccctga attgtacact ttaaaatggt |
| | taaagtggca agttttcact |
| 26641 | aagcagtaaa ttaaattcta ctacaatttt aaaaagacta |
| | aaaaataatt taaaaaagat |
| 26701 | taaatgagat aacgcaaaaa agcattatct cgaaaataca |
| | gctgatatta gtataattct |
| 26761 | tactaagttt taagagtcta aggtgcagga ttctaagttt |
| | aaagggatag gctcttttgg |
| 26821 | ttttttggtt tagttatttg gttttttttt ttaatccatt |
| | atccccaccc ttgggaggcc |
| 26881 | cccagcaccc agtctgcact agaggatggg gcccacctcc |
| | cttttctctc caggcccagc |
| 26941 | cactgaccac cagtaccctg gccaggggca ccctcggtca |
| | ttgccctccg tggcccaagg |
| 27001 | aagggaacag aaacaacagc caagaagaca atagccgccg |
| | ggaagtcctc acatttctgg |
| 27061 | agaaatagag cccattaatg aatgaagttc ctccagcctg |
| | atcggaggac ggggtgctgg |
| 27121 | ggaggcctgg gctaaagggc tcacctccag cccccaccct |
| | ggcagggccg atggtacatg |
| 27181 | ctcactcagt gagggggctc cagaggtctg tgggtacgaa |
| | cccaagggct ggtgcccagg |
| 27241 | ggcaatcagc ttatgtctct gagccttggg aaacagtgag |
| | ggtcagcccg gctccccacg |
| 27301 | tgcttctggg cagctttggt attggagcag gtgcaaactc |
| | Rggactaggg caggaccccc |
| 27361 | tgagaggcga ctgagcaagg ccatcccgac tcatgtttcc |
| | ttggccctgc ccggggcaca |
| 27421 | gcatcctgcc cacatccctg cagccctggc tccttcctag |
| | gggctctgag gaggcagcac |
| 27481 | ttggtcatct ggtcacagtt gctgcagggc agttcttggc |
| | cccagctgta ggtaaagtac |
| 27541 | tgtatgttgt aattttttga aagataacac gttcacacaa |
| | ctcagaattg aaatgccaca |
| 27601 | gacattcccc ctgctccgcc cctttccccc ggatacccag |
| | tttctcccgg aggcagccaa |
| 27661 | tgatctcaga ggctgtatac ccccccagag ttattttatg |
| | catatcaagg aaagtctaca |
| 27721 | tagaggactg tttctggggt acccagatgc agcgtcaaat |
| | gccatggaat actacagtga |
| 27781 | ggacattatc ctttcaagct ttcaaatcag agcaagggaa |
| | aggtcgatgc tagagtttct |
| 27841 | ctagcaccca tgaagccctc tccctttttc tactgagttt |
| | tactttacag gcaacagcag |
| 27901 | gcttcaagct tggggtcatt gtcgggcaac agtatctggc |
| | aagaattcaa tgtctttttc |
| 27961 | tcatagtcat tgtattttgg cctctttcta tttatggcaa |
| | ctgagagaga aagcttattc |
| 28021 | ctagatatat gtatttaagt aaaaaataaa tgaattcatg |
| | gaaacatatt aagcaattat |
| 28081 | ccagataaca taagggatgg caaaaatggt gcagatggtg |
| | gaggggagac aagtagaagt |
| 28141 | tggggtgctc ttgttgaatg tctggctctg aactctagag |
| | gaggccgcag gggctgggca |
| 28201 | ggaaggaggt gaatctctgg ggccaggaag accctgctgc |
| | ccggaagagc ctcatgttcc |
| 28261 | gtgggggctg ggcggacata catatacggg ctccaggctg |
| | aacggctcgg gccacttaca |
| 28321 | caccactgcc tgataaccat gctggctgcc acagtcctga |
| | ccctggccct gctgggcaat |
| 28381 | gcccatgcct gctccaaagg cacctcgcac gaggcaggca |
| | tcgtgtgccg catcaccaag |
| 28441 | cctgccctcc tggtgtgtaa gtatcagtgc atctgtctgc |
| | cctgccaggg gtcttttcat |
| 28501 | ggacacccac tatgccagga gcctccctgg cctgaagcca |
| | gccctgaagc cggctgccac |
| 28561 | actagcccag agagaggagt gccctgggag ggagatgggc |
| | tgagYggagc tgtcatcacc |
| 28621 | ccctcctgac ctcgccttca aggtcaagtt ctttggtRag |
| | aaggtcctag ctgcattgca |
| 28681 | aacagccagg tatagggatt tgtgtttgtc tgagacccag |
| | aatcactggg gttcRagtta |
| 28741 | gggttcagat ctgagccagg ttagggggtt gagtcagggg |
| | gtaaagatta ggaggttggt |
| 28801 | gtatatttgg tgttgggggt cactctatgg ccaaagtcag |
| | gggttgccat gagctcaggt |
| 28861 | gacggaggct ccatcactga ctgttgtgtg actttggcca |
| | gctcccctgc cctctctggg |
| 28921 | cctcagtctc ttgctcaata taataagggt atagggaggc |
| | taaatgatac aatttctaaa |
| 28981 | atagagtatc gccaagttca aaagccagaa ttatagaccc |
| | ccaggactac agacagtgtc |
| 29041 | acagcatcgt ctgggtgagg ctagggttag tgtgcggctg |
| | ggctcagggc tgccccattt |
| 29101 | gctaggatcg tggggttccc atgtgtcagg atccagaggc |
| | tagggtatga tcaggatctc |
| 29161 | tagctggggt cagggtcaga gctctctgtg tcccctagaa |
| | ttgccatcaa ccttaaaccc |
| 29221 | agagggaggc ccagtccaac ccctcagctt taagacctgc |
| | tgggagcctc atctcagaga |
| 29281 | ggctgagtca tggccaaggc cagttggggg tgggagcagg |
| | gggcttggtg tgggcctgca |
| 29341 | gcccctcatc cactgccctc ccctctagtg aaccacgaga |
| | ctgccaaggt gatccagacc |
| 29401 | gccttccagc gagccagcta cccagatatc acgggcgaga |
| | aggccatgat gctccttggc |
| 29461 | caagtcaagt atgggttgca caagtgagtc gggcctcggg |
| | tgtgaccagg ctgggggtag |
| 29521 | ggaggcggga ggaacagcct ggggcttgcc cccagcccac |
| | agggaggaaa ggcagcagct |
| 29581 | gggggactca ggtctctccc cttgatttgg aaccagagcc |
| | tgacaccttc cctaccccca |
| 29641 | ccctccatac cctggtgccc tggggggatt tattggagtR |
| | tatcaacctc tccaacagcc |
| 29701 | cctctaagag tcaggcttca aagggtcctt tcccactgcc |
| | ctgggaagat ggaggtctta |
| 29761 | tttcggggtg aatggggggt agggtagaaa aatctcaaca |
| | aaataagtat tttttaaaaa |
| 29821 | aatgtataaa tgttgttttc ttatagtaca gacaggtctt |
| | gctatgttgg ccaggttggt |
| 29881 | cttgaactct tggcttagcc agtcccccac cccaagccta |
| | aaattagtat cttgacttta |
| 29941 | tttgggatga tggtaacagt caagggtcgg ttgtgggcca |
| | ggtgctttta caaacaaYat |
| 30001 | cctcaccttt agcatatatt tttttctttc ttgtttttca |
| | ttttttgaga cagagtctcc |
| 30061 | ctctgtcact taggctggag tgcagtggca caatctcagc |
| | tcgttgcaac ctccgtctct |
| 30121 | gggttcaagg aattttcgtg cctcagcctc cagagtagct |
| | ggaattacag gcacacgtca |
| 30181 | ccacatccgc gtaatttttg tatttttagt agagatgggg |
| | tttcatcatg ttggccagac |
| 30241 | tggtctccaa ctcctgacct caagtgatcc gcccacctca |
| | gtctcccgaa gtgctggggt |
| 30301 | tacaggcgtg agccaccacg cctggccctt tatctatctt |
| | aatctttatt ttatttattt |
| 30361 | atttatttat ttatttattt atttatttat ttatttattt |
| | attgagatgg agtctctcac |
| 30421 | tgtcacccag gctggagtgc agtggcacga tctcagctca |
| | ctgcaacctc cacctcccgg |
| 30481 | gttcaagacg ttctcctgcc tcagcctccc tagtagctgg |
| | gattacaggt gcccaccacc |
| 30541 | acgcccggct aatttttgta ttcttagtag agacggggtt |
| | tcaccatgtt gacctggttg |
| 30601 | gtcttaaact cctgacctca agtgatccac ccgcctcggc |
| | ctcccaaagt cctgggattg |
| 30661 | caggcgtgag ccacagcgtc cggcctatct taatctttat |
| | catatatttt agcctcttct |
| 30721 | gtaaaagagg ataatgccat cgcccggatg accctgctta |
| | acaaacacgg aaacaggagc |
| 30781 | cgatagaggt ttagcagcct ggcggagctc atgtgcaaac |
| | tcagctcagt atcggaaact |
| 30841 | cagccacctt catggcacct caggctgccc tggagcctgg |
| | gtcccggtct gtgtgtcggt |
| 30901 | ctgaggcccc aagggtgggg cagacattgt tcagggacct |
| | tgtctttgtc ttaaagccaa |
| 30961 | tctcctcctc ctccccggcc ctgctaaaca cgctccccag |
| | gggtggtctc tgctacccgc |
| 31021 | gcacattggg ccttgagtct tggtaaatgt ttctgagcca |
| | ccagggggcg ccagcggtgc |
| 31081 | tggatgccac ttcgcctttt gcaggaaaac tcccgccgct |
| | agcgggtgct gtggggcgtc |
| 31141 | ccaggtctcg cgcagccacg ccctctgtgt gaccttgggc |
| | ttcagtttag gcttctgtaa |
| 31201 | aagagaataa tgggcaaggt ccagtggctc atgcctgtga |
| | tcccagcact ttgggaggcc |
| 31261 | gaggtaggag gattacctca gccagggttc aagaccacct |
| | gtctctataa aaaatttaaa |
| 31321 | aattagctgg gcatggtggt gcgtgcctgc agtcccagct |
| | actcaggagg ccaggggagg |
| 31381 | tcaaggctgc ggtgagctat gattgcatca ctgcattcca |
| | gcctgggtga cagagcaaga |
| 31441 | ctctgttgct aaaaaataaa agaggataat gctatcctag |
| | acctatgtgt tgcaagaggt |
| 31501 | cagacatggc aaaagcgttg agaagttaaa gaaatagtat |
| | ccttaataaa tcagcaataa |
| 31561 | ttcagtaaga ccagactgcc atagactaga tgtcatagtc |
| | attaatatat aactgttatc |
| 31621 | ccaaacgtat atgagtgggg tagaaataaa atatgattgg |
| | ccataactat gattggtcat |
| 31681 | gattaatgtt agatgaagtt ggataatggg taagatgaga |
| | attcttcaca ctattctctc |
| 31741 | taaagcgaat ggaataaaat agaacagaac agaccgggaa |
| | agccagatat atgtggattc |
| 31801 | tccttgctgg gcacagtgtg tcatagtggc ttataatatg |
| | ggatttggag tgggatggac |
| 31861 | ccaggatcaa attcccttct actgaggttt tctgaagcca |
| | cataagctgt ctgagccatt |
| 31921 | tctgtctcag ttcagtgggt acagcaatag tgactacctc |
| | gtgggtagct gtgaggatta |
| 31981 | aaccaggaaa tgtgtataaa ctctaagcac agtgcctgcc |
| | acacagcatg tgtttggtac |
| 32041 | gtgatggccg cttctattgt gtcatattcc atgtgctcaa |
| | gccgggccct gggggagagt |
| 32101 | tcagcaaggg tagagggata tgagagtgaa tgtggcccac |
| | tggcctccct gcctgcagtc |
| 32161 | tctcctctct gatccttgct tcctaagggt cttcactgag |
| | ccccacctct gaccaagtcc |
| 32221 | ccggcggcac aggcagcaca gatcctctgc tgtacctaca |
| | ccaagacccc agggccaacg |
| 32281 | cttctcccat tgtgcagctc cacctctgcc cccggggcct |
| | agctgaggcc tcctccaagc |
| 32341 | tgagccacct actatcttcc aaactcccac cctccatctg |
| | gtgtcacctc ctcccagaag |
| 32401 | ccctctgtat cagtttcctg ttttctgcca taaaaaaaat |
| | cactccaagt ttagtggctt |
| 32461 | aacatgacac aagtttacca ttttacagtt taaaaggcta |
| | gaagtccacc atggccctcc |
| 32521 | Ytgagctaag atcaagctgt cagcagaacc agattccttc |
| | tgggggctat aggggagaat |
| 32581 | taattccctc gctttctgca gcttctagag gcttccccca |
| | ttccttggct tccctccatc |
| 32641 | tacttttatt tagggcgagg gaagcttcct gctctgactt |
| | tctcatttat ggtattctcc |
| 32701 | cctgctttcc agcagctgtg attgtcccaa gtcctgtcct |
| | ctggttctct aggccagaaa |
| 32761 | aactatgatc taccagagct caagaacttg aaagggcctt |
| | ttcccactgt cccttctcac |
| 32821 | agtgagagcc ggagcccagc ttcgtgctag aatctgtgaa |
| | aatggagaaa tgcaccctgt |
| 32881 | gctgttctct tcctccaccc ggcatctccc ctgcagcccg |
| | gctgggtttt tttgtttgtt |
| 32941 | tgtttgtggg tttttttgtt tttttgtttg tttttgagac |
| | agagttttgc acttgtcgcc |
| 33001 | caggctggag tgcaatggca cgatctcgac tcactgcaac |
| | ttctgcctcc tgggttcaag |
| 33061 | cgattctcct gcctcaacct cccgagcagc tggaaccaca |
| | agcatatgcc accactcccg |
| 33121 | gctaatcttt tttttttttt tttatgcatt tttagtagag |
| | atggggtttc accatgctgg |
| 33181 | ccaggctggt ctcgaactcc tgacctcaga tgatccacct |
| | gcctcggcct tccaaagtgc |
| 33241 | tgggattata ggcgtgagcc acggcctggc ccagtctgac |
| | tgttttcact tactctctaa |
| 33301 | tgtctcaggt agtgtttatt gtattctgtg cggaggtcaa |
| | gaatttagtc atccaaggag |
| 33361 | gttagtgtat taggagctta cgcagctatt accagaagag |
| | actccactca aaaatttggt |
| 33421 | acgtggctgg gcgcagtggc tcaaacctat aatcccagca |
| | ctttgggagg ccgaggcggg |
| 33481 | cagatcacct gaggtcagga gttcaagacc agcctggcca |
| | acatggtgaa accctatctc |
| 33541 | tattaaaagt acaaaattag ctgggcatga tggtgcatgc |
| | ccataatctc agctacttgg |
| 33601 | gaggccaagg caggagaatc actgaaccca ggaggtggag |
| | actgcagtga gctgagattg |
| 33661 | cccgctgcac tccagcctgg gcagcaagag caaaattctg |
| | tcacacaaac aaacaaacaa |
| 33721 | acaaaaaaaa cgggttgggc atggtggctc atgcctgtaa |
| | ccccaacact ttgggaggct |
| 33781 | gaggtggggg aatcacctga ggtcaggagt tcaagaccag |
| | cctggcaaca tggtgaaacc |
| 33841 | ccctctctac taaaagtaca aaaattagct ggatgtcgtg |
| | gcgggagcct gtaatttcag |
| 33901 | ctactaggga ggctgaggca ggagaatcac ttgaacccag |
| | gaggcagagg ttgcagtgag |
| 33961 | ccgagatcgt gccactgcac tccagcctga gcaacagagc |
| | cagactcagt ctcaaaaaaa |
| 34021 | aaaaaaaaaa caatggtatg agctttaaat ttctgtagaa |
| | ctgaaagagg attggagatt |
| 34081 | gtagtagggt ctaattctaa attagagcat ttagaataat |
| | gtggagtctg gtcaacatag |
| 34141 | tgagaccccc ccaatctgca caaaaaagac aaaaattagc |
| | caggcagtgt tggtgcatgc |
| 34201 | ctgtggtccc agctattcca gaggctgagg caggaagatc |
| | gttggaattc gaggctgcag |
| 34261 | tgaactgtga ttacacaact gcactctggc ctgggcaaca |
| | gagagccacc ctatctcaaa |
| 34321 | caagaaagaa caaactaaaa aaaaaaaaag aagaagaaga |
| | atttggagca atctagttgt |
| 34381 | ttcctatacc agaagtcttt gccaatctat ttttttttaa |
| | ttttattttt aatttttgtg |
| 34441 | agtacatagt aggagtatat atttatgggg tacatgagag |
| | tacatagtag gagtatatat |
| 34501 | ttatggggta catgagatgt tttgatatag acctgcaacg |
| | ttttgtcaat ctattttttt |
| 34561 | tttggcgaca gtcttgctct gttgcccagg ctggagtgta |
| | atggcgcgat ctcagctcac |
| 34621 | tgcaacttcc gcctcgtggg ctcaggctat tctcctgcct |
| | tagcctccaa actagctggg |
| 34681 | actacagacg cccaccatca tgcccggtta atttttttgt |
| | atttttgtgg agacgagatt |
| 34741 | tcaccatgtt ggccaggctg gtcatgaact cctgacctca |
| | ggtgatctgc cctcctcggc |
| 34801 | ctcccaaagt gctaggatta caggtgtgag ccaccacgct |
| | ggcccaccaa cctatttttg |
| 34861 | atctaacttt acctctggtg gtgtattgat actcttccga |
| | atcaattttc acccttcttc |
| 34921 | atataccatt tacatttcta agaatttgtc catttcattt |
| | catctagtta acctcatttg |
| 34981 | ttgacataca attgttcgta gtattctctc ataattcctt |
| | ttatttctgt aagactggta |
| 35041 | ataatggcct tgcttttatt tctgttttta gtaatttgag |
| | tcttcttctt tttttttttt |
| 35101 | tttgccagtc tagctaaagg tttgttgatt ttgttgatct |
| | tttggaaaaa ccagctattg |
| 35161 | ttttcaccga ttgttgtttt tgttctatta tttcattcat |
| | aggcactaag atcctgatta |
| 35221 | cttccttcct tctgctagct ttgggcttag tttgccctta |
| | tttttcaagt tccataaggt |
| 35281 | ggaaagtttg attattgagt tgagatcttt ctctttttta |
| | aagataggca tttctagata |
| 35341 | taaatctccc tgtgagcacg gttccctcca tcttcagcac |
| | accagggttg actctctccg |
| 35401 | ggcgttcttc cctggtcacc tctccccttc ctctcctctt |
| | ctgcctcctc ttccactttt |
| 35461 | cggtaccctg tgattgtatt gggaccaccc agataaccta |
| | ggatcatctc cccacctacc |
| 35521 | ccaaggtcct taacttaacc atactttcat atgggtaaca |
| | cgagttgagt gtggtaccca |
| 35581 | gggtttgaca tgttgggtaa catatttgca ggttctgtgg |
| | attaggagga cattttgggg |
| 35641 | gccatgattc tatcttccac cctcgcctag acaaaattgg |
| | aggctcactc cttgggctcc |
| 35701 | ctggatgacc cccaacatcc ttcctcactt ccattccttc |
| | ccagcatcca gatcagccac |
| 35761 | ttgtccatcg ccagcagcca ggtggagctg gtggaagcca |
| | agtccattga tgtctccatt |
| 35821 | cagaacgtgt ctgtggtctt caaggggacc ctgaagtatg |
| | gctacaccac tgcctggtgg |
| 35881 | taagcattcc tgtcagctga tgccccatgc cctggccctc |
| | tctgggctgg agggctgaat |
| 35941 | gagggtcctg ggtccttggc tctttccagg ctgggtattg |
| | atcagtccat tgacttcgag |
| 36001 | atcgactctg ccattgacct ccagatcaac acacagctga |
| | gtatgtgtca agcgtcctct |
| 36061 | ggggaagtgg gagctggact ccagggcttg gcctcagcag |
| | agggggaggt tgtgcaggca |
| 36121 | gagggttctg gggccaccaa aggaggccca gcctgggaag |
| | tttgcagggK tggggacccc |
| 36181 | agagctggcc aagctcttga ctggcctggg cagcatgtgg |
| | ataccatctg atagcggagg |
| 36241 | ctgccctgag gtcatgtcgg gtctccctgc agcctgtgac |
| | tctggtagag tgcggaccga |
| 36301 | tgcccctgac tgctacctgt ctttccataa gctgctcctg |
| | catctccaag gggagcgaga |
| 36361 | gtaagtacac caccctgtgg cccccattcc tgctcgtgcc |
| | catcctgtta gtgtgtccac |
| 36421 | ggctccYtcc aggctcaacc ccacacaggg catgcttgtg |
| | ggtggccaaa cctgagggca |
| 36481 | gcaatacctt cagtggggtc acttcctacc ccctcccatc |
| | aatacaccct caaaggctgg |
| 36541 | aaacaacaat aaccaacagc tagtaactaa cagctattaa |
| | gaacttgctg tgtgcaaagc |
| 36601 | actattccaa gcccttttca tgaattaatt gattttgtcc |
| | ttaaaaccaa ccctaggata |
| 36661 | tagattctgt tatcatcccc tttttacata tgggtaaact |
| | gagtcacaga gaggttagaa |
| 36721 | aggaaaagct catatctacg gagtgcatcc tgcattccaa |
| | gcaccacact aactcagaga |
| 36781 | taaaactcta gccaagctaa gtaacttgct gaggacacac |
| | aactcgccac taagggatgg |
| 36841 | gagtaggatt tgaacccagc attctctgac cccagaagct |
| | gagttcctag atactttact |
| 36901 | ctcctgcttc ccagggtggg gctttttgtc ttggccaaca |
| | ccctctgtca aggagctgtg |
| 36961 | ggtaacccca ttgcacagag gaagataaca aggtttggag |
| | agtccctagt catgttacca |
| 37021 | atgccaaacc tggaaggcag aagggaactg gtgggtgggg |
| | tctggagagg agccctctat |
| 37081 | tcaggccatt ttctgactct ggagcagacg gatacatgta |
| | tgaatttgga ctctagacac |
| 37141 | gttctcgtgt gtgtgacagg tgtgagcgtc acaggagctg |
| | ggccctcccg aggaattctg |
| 37201 | ggatggtgcc acagttaatt cttgggtctg aggctccgtg |
| | ttctcagctg caaaatggga |
| 37261 | gtgataattc ttacttcctg agctacaaga gtcagggcca |
| | acagagccat gaacggtgcc |
| 37321 | tggtacacac taggcRctcc atggatgcac aggactggtc |
| | aggggctcat tgtggtgctt |
| 37381 | gctgccttca ggcctgggtg gatcaagcag ctgttcacaa |
| | atttcatctc cttcaccctg |
| 37441 | aagctggtcc tgaagggaca ggtgagtgag gctggctgac |
| | tccctgtggt ccagggccat |
| 37501 | gcccaggagg ctggatccct ttcctccctg cctttccctg |
| | agaaggtgcc actcccacct |
| 37561 | tctccatgtg gccagtcccc tgtgccggtc cccagcactg |
| | ccaccaccac gcagctggaa |
| 37621 | ggaggcactg cctctggcct cctttcctgc ctggaaagca |
| | cctgctctgt ctgccccaga |
| 37681 | tctgcaaaga gatcaacgtc atctctaaca tcatggcYga |
| | ttttgtccag acaagggctg |
| 37741 | gtgagtgYgt ttctgtctgc atgcctcaga agacagcagt |
| | gggagccaga aagccacctg |
| 37801 | ctgcactatg tggccttggg actgtcactc ttcctgtcta |
| | ggtcccatgg gctctatctg |
| 37861 | gctctgacac ttgatgatta gttatgagca tactttggca |
| | aatctctgcc cctttgggct |
| 37921 | gcagcMtcac aagctgtgtg gcgttgggca agtctataga |
| | actcaggaca aatgggtgat |
| 37981 | taagtccaag aggactccaa gattctcctg gaagtagatt |
| | aggaaaaaag ataattagat |
| 38041 | tgctcacatg gctgggcact catccatgta ctgtactctc |
| | ctatgcagta cagagcagag |
| 38101 | ctgggtttca gcccaagtct tggactctgc tctgaaccaa |
| | ccttctagaa gggctctacc |
| 38161 | tacccagaca gacagacttg ggaaaagaga gaatgaaaaa |
| | gtgccacacc cctccccgca |
| 38221 | cacccaggtc ccactttaca gaggggaaca ctgaggctgg |
| | agggttgggt agctgtgtgg |
| 38281 | atgcagggga gcggtgactc agggcaattc ccccatcctg |
| | aggccctgcg ttgatctttt |
| 38341 | cctcctgcag ccagcatcct ttcagatgga gacattgggg |
| | tggacatttc cctgacaggt |
| 38401 | gatcccgtca tcacagcctc ctacctggag tcccatcaca |
| | aggtaggagt tgtgggaggg |
| 38461 | tggggcaggg cccagcttcc ccaggggagt tggtcctttt |
| | ttgtgctctg acaaccccgt |
| 38521 | cccccagctt caaccttatg gcagccaaga gtcctggggc |
| | gctcctcctc attcctgatg |
| 38581 | ctcctccgca ttcctgatgc tgcgaggagg gcaggccaca |
| | gcgacgtgcc cctgacccct |
| 38641 | ctctgcaggc accagggctg cccactacaa ggatcccagc |
| | aaagcaccag ctccttccta |
| 38701 | gagggcttat tcggcttctg tcatcctcta cagcagtgga |
| | ttgtggcccc cYcccagggg |
| 38761 | gtactgacaa aagctttgga ccctctatta cttaggatat |
| | agatttctgt aacaaagaaa |
| 38821 | tccgaaataa tagctgctta aacacaataa acagttattt |
| | ccctctgttg cgttctgagc |
| 38881 | atctgcagcc ccgcgggatc aggaggctgc agggtgtcag |
| | gcacgcaggc cccttccacg |
| 38941 | tgttctcagc cagccccagg catgacctca tcccagggtc |
| | ctggctgact ggcctgacca |
| 39001 | ccacacccac tctgctcaca gcccgttggc ctgaacYtga |
| | tcgcaggacc ccaccctagc |
| 39061 | tgcaagggaa actgaggaat ttatttcagc taaatattga |
| | gaccagctaa aagttaagtg |
| 39121 | gtacagaatg gagggagaga ttggagaaga ctttagggcg |
| | gacccagcca tctttgtaac |
| 39181 | agagcctctc cctggaaaac tggacagaag gatcatttca |
| | gaggttcatg gccactcctg |
| 39241 | aagcctgttc acaacccatg ggctggatga tctagtgggg |
| | cgggggactg ggcagcagct |
| 39301 | tgttttcctg attttggcct ccaggagccg atggtcaatg |
| | gactgccctc tgcaggggca |
| 39361 | gggctggtgg tcagctgggg cggggtggga gctggaggtc |
| | cgtggtcacc agctgccctg |
| 39421 | actaatgtcg ttacttgaat ataaccctgt gaaggcagga |
| | accacgtctg tctggttcac |
| 39481 | ttcccacggt ggttgagaca tagtgggcac tccggaagta |
| | tttgttgaat gagtgaaagc |
| 39541 | cccgctgggg gaaactgggt acagctcttt cctcagtttc |
| | cccatctgca ctctgggctg |
| 39601 | aatgctgggg ctcctcccaa tctccctgaa gctggacctg |
| | agcccagtag ggacacacag |
| 39661 | ggtccagcca gcgtcctggc ttcctccagg gtcatttcat |
| | ctacaagaat gtctcagagg |
| 39721 | acctccccct ccccaccttc tcgcccacac tgctggggga |
| | ctcccgcatg ctgtacttct |
| 39781 | ggttctctga gcgagtcttY cactcgctgg ccaaggtagc |
| | tttccaggat ggccgcctca |
| 39841 | tgctcagcct gatgggagac gagttcaagg tgagtgggtg |
| | gggctgggct gctagggRat |
| 39901 | ccagatggca tgtggtatgt gtgtgtgtgc acacgcatgg |
| | ggaggaggga ggaaactcgg |
| 39961 | aaacttggtg gtgggcaaaa gaactaagct ggagcaatag |
| | cagtgaagtc cagactgggc |
| 40021 | acagtggctc acacctgtaa tcccaatcct ttgggaggct |
| | gagatgtagc aggacgaacc |
| 40081 | gcagacaaaa ctcctcagac actgagttaa agaaggaaag |
| | agtttattca gccgggagca |
| 40141 | tgggtaagac tcctgtctca agagcggagc tctccgagtg |
| | agcaattcct gtccctttta |
| 40201 | agggctcaca actctaaggg ggtctgcatg agagggtcgt |
| | gatctattga gcaagtagca |
| 40261 | ggtacgtgac tgggggctgc atgcaccggt aatcagaacg |
| | aaacagaaca ggacagggat |
| 40321 | ttttacaatg ctctttcatg caatgtctgg aatctataga |
| | taacataact ggttaggtca |
| 40381 | ggggtccatc tttaactacc aggcttaggt caggtaggcc |
| | caggcctggt ttcgggtctg |
| 40441 | gttccttggt ttcgggtctg gtctctaggc gttgggctac |
| | ctgcctttag tttcgcttct |
| 40501 | ctttcttttt ctgagtataa aacaatataa aacaatatga |
| | gagggtctgt ctctcttctc |
| 40561 | tcagagacag gagggtccct ggaggccagg agttcaagac |
| | cagcctgggc aacatgggga |
| 40621 | gacccctgtc tctacaaaaa taaaaataag tttaaaaatc |
| | agctgtgcac ggcRgtgcat |
| 40681 | acctatagtc ccaactactc aggtggctga ggagggagga |
| | tcacttgagc ccaagaggtt |
| 40741 | gaggctgcag tgagctatga tggcaccact gcactccagt |
| | gtgggtgaca gagtaggact |
| 40801 | ccatctcaag aaaaaaatac agtccagcat ttattggagt |
| | caactatgtg ttgccagata |
| 40861 | gatagatgga tagatagaca gagtgatcaa tagatttagg |
| | tatagatata acttggcacg |
| 40921 | tagtaactgt gcattaaata taagttgata ttagacttac |
| | atttattgca gttatatttg |
| 40981 | ctacactttg ttttgttgca ctcatcctgt gaaataagta |
| | ctgttacctc tgttttgcaa |
| 41041 | acaaaaaaac cacaaagctc agagaaggta ggtgacttac |
| | tgaagatcac acagcctgta |
| 41101 | agaggtggcc ccaaagcctg tactcttcac ctatactgta |
| | ctagaaatgc ttaggtagtt |
| 41161 | tccaggctgt gcttgtcatt gaactcatga ggaaactgag |
| | gcccacagag gggaagagac |
| 41221 | ttgtgcgagg tcacacagca tgtgtggggc acagtgggga |
| | gcagaagcca ggcctccagc |
| 41281 | cgggacaggg gttccctgtt ccacaccctg cccagagcat |
| | ctcacatgtt gtctgggagg |
| 41341 | ttgggagttg cgtctgagga ggggtccagt ccttgaaact |
| | gcccttggtc cctgcgaagt |
| 41401 | tttcttctga ggagtggact ttactccacc caccctccaa |
| | cttcctcatt tcttttcagg |
| 41461 | cagtgctgga gacctggggc ttcaacacca accaggaaat |
| | cttccaagag gtaactgccc |
| 41521 | cctgcccctg tgtggggttt atctcacgta ccccaatcct |
| | gctctggctt caagagcccc |
| 41581 | cacacagcca ataacaccac caatggcaac aattataaca |
| | gcgaacacag ctcctactcg |
| 41641 | gttgttcggc atggagtgaa gcacttagtg tgtgtgactt |
| | ccttcagtgc tcacaccgac |
| 41701 | cctatgagtg gggcggtcaa actgtcccca ttttacacac |
| | agggaaactt agtgaatggc |
| 41761 | aaggctgggt ttgagcccag ctctattgcc cccaaagata |
| | aggctccatt ccctgctcca |
| 41821 | tttcccaggc atagRgactt gtagggggct ggaaccccag |
| | gatcaactct gggctcagag |
| 41881 | ggccccagca ataagtgact gttgattact cctgatccca |
| | aagctgactt caggcaagct |
| 41941 | ccttggaggt cgcagcccct tcttgctatg cccagtggca |
| | atgatgttca taatcccact |
| 42001 | cctcagtgca gggttccact aagaacccat gatctcctac |
| | ctcaaatgga cctcatgctt |
| 42061 | tctgagtaag cctccctcag ctttctggtc acctcactcc |
| | ccccacccac tgcaatgact |
| 42121 | tcttcaggcc ttccctgcca tcctcaaatc tccagctgcc |
| | ccctcctgtc taccttccac |
| 42181 | ttccctctcc acacacaacc tgcttaccag agagctgagc |
| | agagccacca acagaacttc |
| 42241 | ccccccacgt cgctgctccc agtcgcaacc cactcaccca |
| | cacctgcgcc ttcctgcggc |
| 42301 | cgttctcctg ttctaacaga ggacRgtccc tcttcccttc |
| | tgaactcagg ccgtccagcc |
| 42361 | ttccccggct ctgtgatgYc tctctccYtg cacatacaca |
| | actcacaata ttgggcaggc |
| 42421 | ccctgaccgg cYcttctccc ttagcccctt gtctgagtcc |
| | tgttcctttg atgtcctatt |
| 42481 | tctagtcttt ccatttctct ctgtctctac ctcctcccca |
| | ggctaccatt atctctagtg |
| 42541 | gggacccatc tgtagctccc ggtgggtctc cctgtgtcct |
| | gataccctcc aataatctgt |
| 42601 | tccccacagc agccagaaca atcttttcaa gaaataaaac |
| | tggtcattca cctgcctgct |
| 42661 | taaagccggt gtaggataca ggctaaacgc tctcccatgg |
| | cctccagggc cctgcatttt |
| 42721 | ctcatcctgt ctacttcttc atctagttgt ttcccaccca |
| | gcgtaattgt aactgtttag |
| 42781 | ttgaaagttg acgaggtctt ttgtcagctc tagaaatttc |
| | ttttcttttc tttctttttt |
| 42841 | ttttttttaa gacagagtct tgctctgtgg cccaggctgg |
| | agtgcagtag cacaatctca |
| 42901 | actcactgca acctccacct tccgggttca agcaattctc |
| | ctgcctcagt gctgaaatta |
| 42961 | caggtgtgag ccacagcacc cagcccatca gctctggaaa |
| | tttcttcatc actctctctt |
| 43021 | ccatgattgc cccatcccta ttttctccat gcctacctcc |
| | tggaatttcc atgagacaca |
| 43081 | cgttgggcca tctcattcca ttttccgtgt ctcttagttg |
| | ctctgtcgtg ttttccatct |
| 43141 | cttttatctc tatcctaaat tctggatagt ttcttcaagt |
| | atctttttag ttcattaatt |
| 43201 | ctttttacag ctgtgtctta tttgtttact tacccactga |
| | gattttcatt ttattttatt |
| 43261 | taattaatta atttatttat ttttgagaca gagtctcgct |
| | cttgttcccc aggctggagt |
| 43321 | gcagtggcgc aatctcagct cactgcaacc tacgcctcct |
| | gtgttcaagc aattctcgtg |
| 43381 | cctcagcctc ccgattggct ggggttacag gggtgcacca |
| | ctgtgcccag ctcatttttt |
| 43441 | tgtattttta gtagagacgg ggtttcacca tgttggccag |
| | gctggtcttg acctcctgac |
| 43501 | ctcaaatgat cctcctgcct cagcctccca aagtgccggg |
| | attacaggtg tgagctcccg |
| 43561 | caccccgccg gccctttctt tcttttgagc tgagctatgt |
| | atttaatgaa accttttctt |
| 43621 | gtgtatccag cRgttcagtg gtttcttggg gcatgtgatc |
| | tccttgagcc cagtcccaca |
| 43681 | ggctgtaatc gaaggtcctc aggccacact ccccagcccc |
| | gctggctgcc tgcgcattcc |
| 43741 | ttatcttggc catgttcctt ccacagggct cttgtccaca |
| | ctgtcccctc tgccagaatg |
| 43801 | gtttgccctc ccccatgcca cctgctcctc ctcctgccac |
| | ctcagggaag gcctctccca |
| 43861 | cctccctgac taggtcaggt cccctcgtgc cacagcctRg |
| | tgtgtctctc ctttaaatac |
| 43921 | tcctcagctg cagttttacg ttctgttctg ggatgccctg |
| | gaaaatgctg ggctcctgca |
| 43981 | gatatggccc gcagctcctg cgctctctgc tctatcccca |
| | cccctagccc agctgctaca |
| 44041 | gcagcttccc tcggagggtt accatgcagt aggtcctgtt |
| | ctaagctctt tccacagatt |
| 44101 | atctcattcc atcctcagga caaccctatg aggtaggatc |
| | tatgattatc cccattttac |
| 44161 | agatgaggaa agtgaggccc agaaggctaa gtgaaggcat |
| | cctcagcaca tcctgagaga |
| 44221 | ggagttcagg gtaggaatag cttcactagc acacagatga |
| | gaaactgagg ccYagagagg |
| 44281 | acaagagtcc taatttgcac agcctggggc ctgggcacta |
| | ccccgagcta cttccttttc |
| 44341 | cccagccctg gggcccggag ccagctttgt ccttcccatc |
| | tccgagggca tggactgctg |
| 44401 | ctgccctgat gggcccctgt cctggccatg ggacccctgt |
| | cttccacagg ttgtcggcRg |
| 44461 | cttccccagc caggcccaag tcaccgtcca ctgcctcaag |
| | atgcccaaga tctcctgcca |
| 44521 | aaacaaggga gtcgtggtca attcttcagt gatggtgaaa |
| | ttcctctttc cacgcccaga |
| 44581 | ccagcaacat tctgtagctt acacatttga agaggtgagg |
| | cgggtgcagg gagaggtggt |
| 44641 | ggtgggggaa cctgactcac atatgggccg cagagggcag |
| | gggcctgggg gtctctgaag |
| 44701 | cctccagatc cttctcacca cctctgctgg cactggttgt |
| | ctcttgcaca tggctcctta |
| 44761 | caatcaaaat cacatcatgc aagtaacgag ggggtacaca |
| | cgtggtttcc acagcttagg |
| 44821 | taatattttc tctcttttct tatttttatt ttttttagag |
| | acagggtctc actctgtcat |
| 44881 | ccaggctgga gtgcagcctc acactcatag ctcactgcag |
| | cctcgaatcc tgggctcgag |
| 44941 | tgatcctccc acctcagccc tcctgagtag ctgggaccac |
| | agacaaacac caccacacct |
| 45001 | ggctaattta aaaaaaaaaa tttccttaga ggcggggtct |
| | tgttatgttg tccaggctgt |
| 45061 | tctcaaactc ctggcctcaa gcaattctcc tgccttggcc |
| | tcctaagttg ctggggttat |
| 45121 | aggcatgagc caccatgcca agcatatgtt ctttcattct |
| | tatttttatt tattatttat |
| 45181 | ttatttactt atttattttt ttgacacaga gtctcactgt |
| | gttgcccagg ctggagtgcg |
| 45241 | gtggcacaat ctcggctcac tacaacctct gcctcccagg |
| | ttccagcgat tctcatgcct |
| 45301 | cagcctccca ggtagctggg attacaggca tgcgctacca |
| | cgcctgacta attttttgta |
| 45361 | tttttagtag agacagggtt tcgctatgtg ggcccagctg |
| | gtctgagact cctggtttca |
| 45421 | agtgatctgc ccgcctcagc ctcccaaagt gctgggatta |
| | caggcatgag ccaccacgcc |
| 45481 | cagccaatat tctcctcctt aagctgagca gtggacaatg |
| | gtgtttatta cccgagtcca |
| 45541 | caacccttta ttcattaggg ccacaagggt ttccaaattc |
| | agaacttctg tgaatttgta |
| 45601 | aaagtagtat agtgtataga cactggatat attgcagaac |
| | accccaatag ggtgtggggt |
| 45661 | ggcaccccac aatcaattct cctcaaatgc ctcaattggc |
| | atttctcctc atatgaagca |
| 45721 | agataaatac catacatagc ttcctagaca atggccaggt |
| | tttgccaaca aaacttaaga |
| 45781 | aaaaacttgt agttttcaga tcctgtttga ttttataatt |
| | acaaataagg aattaagaat |
| 45841 | cagtattatc tttaatgctc cttagaatat cttcaatttt |
| | tttttttgag acagagtctc |
| 45901 | actctgtcac ccaggctgga gtgcagtggt gcaatctcgg |
| | ctcactgcaa cctccacctt |
| 45961 | cctggttcaa gcgattcgcg tgcctcagcc tccccagtag |
| | ctagctggca ctacaggcgc |
| 46021 | gtgccaccac gcccagctaa tttttagtag aggcggggtt |
| | tcaccaggtt ggccaggcca |
| 46081 | gtttcaaagt cctgacctcc agtgatccac ctgcctcagc |
| | ctccaaaagt gctgggatta |
| 46141 | caggcatgag ccactgtgct cggccaatat ttcttaatac |
| | attaagaact aaagacaaga |
| 46201 | gcgatacaat cacattgtgg aaaatataga aaaatagaaa |
| | gaagaaaacc actgtcacca |
| 46261 | taaccccctt ctccaggggc cttcgatgtg gtgactgggt |
| | gcatttcctt ctggtgcttt |
| 46321 | gttattttta aataaatttt ttatcttgga gtaactttat |
| | atttgaataa aagttgcaaa |
| 46381 | atacagagtg ttcccatata tcccccaccc acttcccgct |
| | ttgtccacat cttacataac |
| 46441 | catgctacag tgtcatagcg gagagattaa cattggtgca |
| | tgactgttaa ccaaactcta |
| 46501 | aactttattt gaatctcccc agtttttcca ctgatgatac |
| | cctttttctt ttccaagatc |
| 46561 | tgatccagga tagcacattg tatttagccg ttcttttttg |
| | ttgttgtttg tttgtttgtt |
| 46621 | tgtctgtttg tttctgccca ggctggagtg cagcggtgat |
| | cctcccacct cagcttctcg |
| 46681 | actagagatg tgcactacca cgtatttttt tgtagagaca |
| | gggttttgcc acattgccca |
| 46741 | agctggtctc aaatccctgg gctcaagtga tcctcccacc |
| | ttggcctccc aaagtgttgg |
| 46801 | gattataggc atgagccact gtgcccagtt tatttagcct |
| | tttaaatatc tctttaattt |
| 46861 | ttgtattact ttccttttca tttttctgtt ttattccttt |
| | tggctttctc atgtggatgt |
| 46921 | agacatatta ttttatttac ttattttttt gttgttaaga |
| | atctgaaaca ttgagcattt |
| 46981 | gttaaaaaaa agaattattc sgccgggcac ggtggctcac |
| | gcctgtaatc ccagcacttt |
| 47041 | gagaggctga ggtgggtgga tcacatggtc aggagttcaa |
| | gaccagcctg gccaatatga |
| 47101 | tgaaacccca tctctactaa aaatacaaaa attagccgga |
| | catggtggca catgcctgta |
| 47161 | gtcctagcta cttgggaggc tgaggtagga caatcgcttg |
| | aacctgggac gtggaggttg |
| 47221 | cagtgagctg agatcgtgcc actgccctcc agcctgggca |
| | acagagtgag actgtctcaa |
| 47281 | aaacaaaaaa agaaaagaaa agaaaaagaa agtgacttct |
| | caggtcctaa ccccaaagcc |
| 47341 | acaggtgctg gggaactttc ctcggttttc agaagagcag |
| | tagctaagcc tggttcccgt |
| 47401 | gtcatccttg cctctccagt ccctcagtgg aaagaatcag |
| | gggccctgag ctaggagggt |
| 47461 | tgctctctgc ttcgggaaga gccctggctc acagcaaatt |
| | tggtttctct ccccaggata |
| 47521 | tcgtgactac cgtccagScc tcctattcta agaaaaagct |
| | cttcttaagc ctcttggatt |
| 47581 | tccagtatgt gctgcagaga agagaggggg cggtcaactc |
| | cgcaaacctc tccctggccc |
| 47641 | cttggagtca ggcacagggc ggggtgttgg tggggaaatg |
| | tggccccttt cYtctggggc |
| 47701 | atatgggctg actgcaggga gataagaccc tgcctagata |
| | gaatcttcgt ggggaagaag |
| 47761 | gggctccagg aagaatggag ggctgccagg aagaagggcc |
| | tggcaggagg agagcgctgc |
| 47821 | ccgagcaaag gcctggccgc cagaatagca aatctcaagg |
| | gaatagcaaa tctcaagaga |
| 47881 | gtgccccaaa gggcctgagc tatgagacag aagcactggc |
| | tgctattctt agagtttctt |
| 47941 | tcccagggga tgttacagga gggggcccaa tggagggtca |
| | aattatcatc gcttttttat |
| 48001 | ttcaggatta caccaaagac tgtttccaac ttgactgagg |
| | taggtagtct tggatagact |
| 48061 | gggggaaata agtcctgtgg gacctcctgc cttaaagaaa |
| | gcaggcggag ggccctaaag |
| 48121 | gaaatcaggc aaccagacca aaagaatgtg gaccaggtgg |
| | tccatgctgt gtctcttgtg |
| 48181 | acccttcttc tccctgccat gtcttttggg agagcccttg |
| | tgttgcaaaa atgagagtgt |
| 48241 | ggtggtatgg attggggttt aggcagaaca gtactggcca |
| | agcagcgcct ccctggacct |
| 48301 | caattttccc tctgtggaat gggctagcaa tcctgggcct |
| | ccccagggcg aaggaaagac |
| 48361 | cactcaggaa gggcaccgtc tggggcagga aaacggagtg |
| | ggttggatgt atttttttca |
| 48421 | cggatgggca tgaggatgaa tgcttgtcca ggccgtgcag |
| | catctgcctt gtgggtcact |
| 48481 | tctgtgctcc agggaggact caccatgggc atttgattgg |
| | cagagcagct ccgagtccRt |
| 48541 | ccagagcttc ctgcagtcaa tgatcaccgc tgtgggcatc |
| | cctgaggtca tgtctcgtaa |
| 48601 | gtgtgggctg gaggggaaac tgggtgccga ggctgacaga |
| | gcttcccatt tcaccttgtg |
| 48661 | ggcccttccc aggcagagct tcaggtgccc ctcttcccag |
| | tcattgatac ttagcggtcc |
| 48721 | tggccccctt tcctctccct gctggtggYa ttgcacgcca |
| | atgactcggc cagatgccca |
| 48781 | gacccctgtt cttggtttac ctgcagaata ttatctttgc |
| | caccccgcgg gatggctcaa |
| 48841 | cccactttca ggatgcaggt ctcctaatag caacctgata |
| | tagcagaaag acccctgggc |
| 48901 | tgggagtctg agacctagtt ctagcccagc cctgaacctc |
| | agtttccctt tctgtgaaac |
| 48961 | aagaatgttg aacttgatga ttcccaattt tccttttgac |
| | cttgaaatgg tagaatattt |
| 49021 | atccctttga ggtgactcgg atggtagact ctcagacacc |
| | atagcacacg tgtgctgggg |
| 49081 | gtattttgga ccaggctctg ctgagagctt tctgctccct |
| | tccccacaga gtatgttgag |
| 49141 | ctgactgtgc tttccttttg aacatctgct tgtccacatg |
| | gcttaggtag gagaggaagg |
| 49201 | gcgtggaaac tggaatgatc ctagtggggt gtcttggcat |
| | ctcttggcct cattttcccc |
| 49261 | atctgaacca tgaagctaaa actaggggat gtggattaaa |
| | tggttcctac aactacttgc |
| 49321 | aaggagacca ctctgtgtgg ttgcaaagaa cactttgaga |
| | agctgtgtgg gaaagtttcc |
| 49381 | ttcctagcag ggtagactca gctaactgca ggtcatgtgg |
| | ccattgtgga tgggttggga |
| 49441 | gctcaagttt ggggcagaag ggaatttttt ttggcagcag |
| | agtggcaagc cctgccgcca |
| 49501 | ggcaaactct gctcttcctc atcctcagaa gcacttgctc |
| | actctgctaa atcaaagtga |
| 49561 | aacgcatgtt tacagaatat tggtccaaaa gggtctcagc |
| | atctcccact acccagggtg |
| 49621 | gcagagcctc gggccggcct tgctccccaa gaagggctga |
| | ctggggctct gtcccctgcc |
| 49681 | ccagggctcg aggtagtgtt tacagccctc atgaacagca |
| | aaggcgtgag cctcttcgac |
| 49741 | atcatcaacc ctgagattat cactcgagat gtgagtacaa |
| | agcccccctc accagcccct |
| 49801 | gttcctgggg agagaggccc agacaggatt cctggggtga |
| | ctgggggctg ttggggagac |
| 49861 | agacagaggg gcctctacca gcttggctcc ctcctggtgg |
| | cctgggagtc agcccagctc |
| 49921 | Rcccctctct cctactgccc ctcccttcag ggcttcctgc |
| | tgctgcagat ggactttggc |
| 49981 | ttccctgagc acctgctggt ggatttcctc cagagcttga |
| | gctagaagtc tccaaggagg |
| 50041 | tcgggatggg gcttgtagca gaaggcaagc accaggctca |
| | cagctggaac cctggtgtct |
| 50101 | cctccagcRt ggtggaagtt gggttaggag tacggagatg |
| | gagattggct cccaactcct |
| 50161 | ccctatccta aaggcccact ggcattaaag tgctgtatcc |
| | aagagctgSg gagtccttct |
| 50221 | tctgtggctg gcgggtagag ggRgggggaa gggattgtct |
| | caccagtgcc gtccacctct |
| 50281 | tttcagccct tccaaggcag ctgcccccaa accctccaag |
| | cttcatgatg actggaggaa |
| 50341 | gaaatccaaa cttctctcct tgggactcac gatcctccct |
| | gatcaggtcc ctggatacct |
| 50401 | tccaaattta tcccctttaa cccagcactc tccttgtctg |
| | accagcttcc tgcaggagct |
| 50461 | gtcacacact ctctgtccct tggggttcca gtgctggaca |
| | gaaagtgagg gaggaccagg |
| 50521 | gcttcagatc ctgaggatgg tgggagggga aggggccctc |
| | agaggtctgg tttctggtag |
| 50581 | aaatggagga agacagagcc tttaaaaagg caaacgactt |
| | tattccagaa gataaactgc |
| 50641 | tcacgagcct gagcttattt ccagctcacc tttttttttt |
| | tttttttttt tgagacggag |
| 50701 | tctctctgtg acccaggctg gcatgcagtg gcacaatccc |
| | agctcactgc aacctccact |
| 50761 | tcccaggttc aagcaattct cctgcctcag cctctcaagt |
| | atttaggatt acaggcaccc |
| 50821 | gccaccacac ctagctagtt tttgtatttt tagtagagac |
| | ggggtttcac catgttggcc |
| 50881 | aggctggtct cgaactcctg acctgcagtg atccacctgc |
| | ctcaaccccc caaagtgctc |
| 50941 | atgttaaata tggtgaaccc caagtttttc ttcaaagaat |
| | cagtatgtct gtatgttcag |
| 51001 | ctctcttatt ctttgattct ccattttaaa gtttaacttc |
| | ctggttctct tcgccccctt |
| 51061 | gcttctaatt tcagtaaaca accttttcca tcagttttat |
| | tcagtagttc acacctgttc |
| 51121 | ccctggtcac ttgctccatc ctgactcatc ccagtcacct |
| | gctttgacct gagtcacccc |
| 51181 | tggtcacctg ctctgatgta agtcaccttt agttacccgt |
| | tcctaactgt ccttcctgcc |
| 51241 | aaactactca cctcgccact ctggctcata cctctgctct |
| | ctttaaaata gccaatcgga |
| 51301 | attagattag accgtgcggt ccaaccttag ccaacagggg |
| | aacaacatag cagcagggat |
| 51361 | tacctgggtc aggaataaga accccttccc ctcccttgtt |
| | caactgtgct ctcaccattg |
| 51421 | ttccatctat gaggagcacc ctttctacag aaactaaaaa |
| | ttgcttgctg agaaaattaa |
| 51481 | atttatgttt gagtgctatt tctttgcagc agtggagaac |
| | aagcattcct aacactggga |
| 51541 | ttgcaggcat aagccaccgt gcccagcttc cagctcacct |
| | tcttatgctc aaattgtgtc |
| 51601 | ttctccttcc caccctcaag ctacaataca aaaagtttct |
| | tccaaccctc tcagtcttgg |
| 51661 | aaggtagcag gctctatttt ctctgggtgt gtaaagaaaa |
| | tgcctctccc acattagcca |
| 51721 | attgagacgg gaggaagcgc agcaagatgc tggctagtgc |
| | aaggtccctg gggtagaaag |
| 51781 | aatcatgatg gattcaagaa cagtcaagag ggctggtgtg |
| | gccctgaaag caaagaacta |
| 51841 | ggggcgagag atgaggccca accagcaggc agggtcacat |
| | aacaaaggtg ataaaagggc |
| 51901 | ttggccttta tttcagcagc caggagagac agccaggagg |
| | ctggggctgt ggggagctca |
| 51961 | gaagtaagat gccccttcca gtctgggaat atgtgagggg |
| | agggcaccat cttctgagcc |
| 52021 | aggtttcttc ccagcccttc cctgcccttc caagtgcttt |
| | ctgaggcctg tttctcttcc |
| 52081 | tcccctgggg cctaatgcaa gtgtctgact cactaagcag |
| | gagcatcatg ggcgggatgg |
| 52141 | gcggctctta aagggccctg gcagaaggag tcagtgtgat |
| | ctcgccagtc cctgagtcca |
| 52201 | acgtgggaat tctgggtctt gggatattgg cctttgaggt |
| | ggtgacacca gttaggagcc |
| 52261 | acatcccacc caagaatgtc cccttgaaga ggctgggaag |
| | gcccaaggcc cccaccttga |
| 52321 | tgctacaatg acagaatcag gataacttgg cagccctaag |
| | gcagctcttc cttgaatgga |
| 52381 | aacttttatc tgcatgaggg gcagacagag aagtcacttg |
| | gagtcttaac ccttgtagac |
| 52441 | cccagcctga ctgggactcc agaaggtccc agggctctgc |
| | tccagaaaga agggtagggg |
| 52501 | catgccccca gcttccttca gtgaccttgg agaggtctcc |
| | ccagggctga gctgggaggc |
| 52561 | acttccatgt cactgtctca tccaaggagt gcgattagca |
| | ggagatatgg agtccagagg |
| 52621 | gtgagaggct taccaaaatg ggactgacct cYgagcccct |
| | ctcccaaatt ccagcccctc |
| 52681 | catccaagcc tagaagctct gacctagtcc tattgaagtc |
| | ctcctggcct gggtgtgtgt |
| 52741 | tggggggaat cctggctcca acccagttag gatcatttcc |
| | ataaagaagg ccacacctgc |
| 52801 | tgcttcctct ggggaaaaac aagtgctccc caaggccagc |
| | accaggcaga agcacatcct |
| 52861 | gccacctggg ctgctgacct tgggcaggat ggggcccggg |
| | tgtttggcaa ataggaaggg |
| 52921 | tagagggaag gcctggcttt cagacctggg tctaatccca |
| | gctcccccca accccagtta |
| 52981 | ctaattctgc catcctgggg cccctctgtg cctccatttc |
| | ctcttctatt caatgggatg |
| 53041 | ataatacttt ccatgtagga ttctggcaag ggctgggtta |
| | gcccagagga ggagcccatg |
| 53101 | gtggccaaga gcaggggctg cagagctggc ctgcctgggt |
| | ccacccttct gctcctctgc |
| 53161 | ttctgtgtga tcttggccaa attatttagc ctctctgtgc |
| | ctcccttttc tcatctgtaa |
| 53221 | aaagaaagat aacagttgcc atgaagagta aaggagaggc |
| | cgggcatggt ggctcagacc |
| 53281 | tgtaattcca gcactttggg aggctgaggc aggggtatct |
| | caggagttca agaccagcct |
| 53341 | ggccaacatg gtgacccccc ccatctctac taaaaataca |
| | aaaaaattag ctgaatatgg |
| 53401 | tggtgtacac ctgtagtccc agctactctg gaggctgtgg |
| | caggagaact gcttgaacct |
| 53461 | gggaagtgga ggttgcagtg agctgatatt gtgccactgc |
| | actgcagcct gggcgacaga |
| 53521 | gtgagactgt cttaaaaaca aacaaacaaa aagattaaag |
| | gagagagcgg ctgcattttg |
| 53581 | ggaggccaag gcgggctgat tgcctgagct caggagttgg |
| | agaccagcct gggcaacatg |
| 53641 | gtgaaacccg gtctctacta aaatatgttt ttaaaaaaaa |
| | ttagctgggc atgcacctgt |
| 53701 | agtcccagct actcgggagg ctgaggcagg agaattgctt |
| | gaaccggaga ggtggaggtt |
| 53761 | gcagtgagct gagatcatgc caatgcactc cagcgtgggt |
| | gacagagcaa gactccatct |
| 53821 | caaaaaaaag gagagagcac agtgtctggt atatcctaag |
| | tgctccacga gtgccagaca |
| 53881 | ttggttatac ttcatggagg tgaggtacac agaggtgtct |
| | aggtcatgcg gactcactcc |
| 53941 | atcctccagt taaccatgac tccctcccca cccacctcct |
| | gagttctctc tgctgtcaaa |
| 54001 | ctttcctctc atatctccag tttcccaccc aaaagtccaa |
| | acactcaaga ttccttctca |
| 54061 | tctccaaccc ataaaatctg atcggcttct ccacactcaa |
| | gtgtaaatga ctgatggcca |
| 54121 | tttgattgac actggagggg ctggcacatt tttagcacat |
| | ttttagccaa aggtatgatg |
| 54181 | tgctgatagc ggactctcag gccacaagcc agtctgagga |
| | cagggaaagt ttgaggaggt |
| 54241 | cactcttcca gaaccctttg gtgataacct ttctggcagg |
| | cctggtaccc ccagagtgaa |
| 54301 | aagggctcca gggtcaagac ccagctctgc ccctcattgc |
| | tgtgtgacct tcagcaactc |
| 54361 | acttgattta tctggacctc agcttcttca tctgtctaat |
| | gggataataa tctttgtcct |
| 54421 | gccctcctca cagggctgtt cagaattaaa tgcatctgaa |
| | aatgatctgc atttgtgtct |
| 54481 | tggactgtgg ttatttgttc aagtgtctgt ctaccctgcc |
| | tgcacaggga gcgggtcagg |
| 54541 | gtcttccctg ggacttcatg cacaggtctt tcctgggact |
| | tcatgtagga ccagccatcc |
| 54601 | cgtcagtgct cagtgaacat gagctgcttc cctgtgggat |
| | gtctgggagg tgagtggaag |
| 54661 | gccttcctag caggcacata ctggaacata atcaatccct |
| | cccatgcata cagctcacac |
| 54721 | ccacattctt atccattccc accatgcccc tcagtggcct |
| | ggggagacta ggaatacccc |
| 54781 | acttgacaga taaggaagct gaggccaggg gaggtcaagg |
| | catttgtctg aggttgcaca |
| 54841 | accaggagtg gtgaagctgg gagtcacaca caaggaatca |
| | ctgcaaagtc tgtgtccttc |
| 54901 | ccactctgag taggacttga accagggatg tccgatgctg |
| | atgcttgtgt cctgaccact |
| 54961 | accttgcagc gtcctcaccc acacccaccc caggcctggg |
| | aggcagggga gcaccagcgg |
| 55021 | tctgggagtg aaagttgctt ccctgtggga tgtctgggag |
| | gtgggtagaa ggccttccta |
| 55081 | gcaggcacag ctgcccaccg gatgagtatg tccaggggga |
| | gaggagcccc ctgtcccagg |
| 55141 | atgtgggcaa aaacctggag aagctgaagc tggactttga |
| | ggatggcccc tgacYccatc |
| 55201 | ctgggacagc cacctcccct tccttctgcc ccctccagtc |
| | cctcctctcc ttccctgctc |
| 55261 | tccctcctac ctctttccat ctcccccttt ctgcctgtga |
| | ttcctcctgt gactggggca |
| 55321 | agatccttca cctctgtcct ccctgagcct cagtggctca |
| | cctggaacat agggtgatgc |
| 55381 | ctgcagaagt attttagggt tactttatga gataaattgg |
| | tagactgttt ttcccgggtc |
| 55441 | agccagccag gggttcggtg aataggagcg aacgctgctg |
| | ccattccctt ctcccctagt |
| 55501 | tcatcctgga ggctctccgc tctccccctg gttctaagtc |
| | ccctcctcct gcaccgtatc |
| 55561 | ccccctccca tccaacccag tccccactga ggcactgaga |
| | cagggtcttg gcccagggtc |
| 55621 | cccacccacc tgagctccgc atgtgagccg accttcaccc |
| | gcctgcctca gtttcccgtc |
| 55681 | cacgaaaggg gtgagcgccc ccacgtctcc tgggactgtc |
| | cttgggtccg atgcggggat |
| 55741 | gtggggaggt gccagggtgt gggcgcagca gggagcgggt |
| | gtgttaggcc cccgcccatc |
| 55801 | ccgcgcccga gccccatctg gctcgggctg gcacctcgaa |
| | tccacgtgat ttctcggcag |
| 55861 | cagccgccag ttccatgcac tggcggagca gctctaggcg |
| | gcggcttcta ctttcagttt |
| 55921 | cgtgcagagc gcggaggagc cgcgagcgct gagggtgagt |
| | gccgggagct ctgagggtga |
| 55981 | gtgccgggcg tgccgcgggg ctgcgggacc cgggctgggg |
| | cgagcggaag ggagaggatc |
| 56041 | ggggttcgaa ttctgcacgg agaggggtgg aggggatgtc |
| | agaggccctg gagcgggagg |
| 56101 | tgcgggtggc cgggtggctg gccgatggat agctgggtaa |
| | gcggacaggg cgccagtggc |
| 56161 | ccggcagcgc gcacagcatg cgccccggca gtttggagga |
| | agagttgccg gcccgcagag |
| 56221 | gatggcagat gagggtcgcc tggatggggg gacacagtaa |
| | gcggctgtgc tcccctccct |
| 56281 | gcgcagctcc tgtcacctac cggatccacc cactctctct |
| | gtcttttact ttgcaacttt |
| 56341 | tcattgattt taaaacaatt acggaagtta cactgttcat |
| | tgtaYgcaat ttagactttt |
| 56401 | tggcctaaac aactttttat tgtggaaaat tccaaacata |
| | tacaatagta gaaggaatag |
| 56461 | aattaggttg agccttctgc acctgcgggt tttcatataa |
| | tgaagcccca cctatccacg |
| 56521 | ttcagcttgg acagtcacga ccccaaggcc acctactcac |
| | gaccccaagg ccacctaggt |
| 56581 | agcccaatct ccaccgctgg ttattttgaa acaaatggca |
| | gacagcatag catctcaact |
| 56641 | gaggtatttc agaatgtact aaaagataag gcatctttta |
| | aaaacaaaac cacagtacca |
| 56701 | ccagcatact acaaacattt taatagttat tccttaatgt |
| | caaatctcca gattatttct |
| 56761 | aaggaacatt taacctcata agtgataggt gaatacgtgg |
| | gtgttgtttt gttacttgct |
| 56821 | ttctgcattc ccatgtggtt ttcggtcact attctcaccc |
| | agaaagccag gacctgaaac |
| 56881 | tggctggggt gatcattcga ggaggtcgga agaggctcct |
| | accttcagtg ccgtttgcgt |
| 56941 | ccattttata agttggagtt cttatgtttc cttttttaaa |
| | aggttgaagt ttctgcagac |
| 57001 | ctaaaaagca gtttgaaaac tggcagttcc cagcctctaa |
| | aacccggggg tagcaggagg |
| 57061 | cagagagtac cccggggcca gatcctggag cctcattgtc |
| | ccttcaaaYc accttccgtt |
| 57121 | tctctaaggg agaaataaac cttgaaaagc catgggcttt |
| | tcagatgtat ctagatgtat |
| 57181 | ctgatgcttt tcaggtgtat ctagactccc atccagcgtg |
| | ctttctgcaa aacttactga |
| 57241 | accatagccc agaatggcca tccatgatgc ccacccgcct |
| | tgtctcctga agcacattgc |
| 57301 | caggtggacc tgcacatggt gtggacggca gacagactgg |
| | ggctctcaac aactggtatt |
| 57361 | tgctggatga ccatgaacta accattcaat ttctcgaaac |
| | tttggtttct cgtctgtaaa |
| 57421 | atgaacatat gcaaatctat cctacaaagt gtgttttgtg |
| | gattgaatga gatgatacac |
| 57481 | gcccacccag tgccttgatc aaccttaaaa tgctgcacaa |
| | gtgtgagggg agatggatcc |
| 57541 | ttgaagaaga cagacaagca gaggggaaag atcttggaag |
| | agtttcacaa tatgctgaac |
| 57601 | tttattcctg gatcagaYac agagctgtcc gggtgtccac |
| | agggccgggt cagcctcctc |
| 57661 | agaccccaac acacataccc tcttcccctc tgaggaaaat |
| | tcagcaaaac ttcttttgct |
| 57721 | gttgttgttc tttattcctt tttttttttt tttttttttt |
| | gacgaagtct cactctgttg |
| 57781 | ctcaggctgg agtgtagtgg cacgatcttg gctcactgca |
| | acctccatct cctgggttca |
| 57841 | agcgattctc ctgcctcagc ctcccgagta gctgggatta |
| | taggcgcccg ccgctacacc |
| 57901 | cagctaattt ttgtattttt agtagagatg gggtttcact |
| | atgttcgcca ggctggtgtc |
| 57961 | aaactcccaa cctcagatga tccacccgcc tcggcctcct |
| | gtgggattat aggcgtgagc |
| 58021 | catcttgccc ggactccttt tttttttttt tttttttttt |
| | aatagacatg aggtctcaaa |
| 58081 | ctcctggcct caagtgatca gtctgcctct gcctcccaaa |
| | gtgttgagat tatgggcgtg |
| 58141 | agccaccaca cccagccata acttcttagc atgacttttc |
| | ccatttgtta tgagttcaat |
| 58201 | tgtgcctccc cacaaaagat atgctgaagg cctaaccctc |
| | ggcgcctcct aacgtgacct |
| 58261 | tatttggaaa tagggctgtt gcagatataa ctggtgaaga |
| | tgaggtcata ctggagtagg |
| 58321 | atgggccttg aatccaatag gaccggtgtc cttataagag |
| | aggagatgga cacatgaaga |
| 58381 | cagagacata cggggatcag acaatcatgg acggaggagg |
| | aagagagtgc ggcgatgcat |
| 58441 | ctaggagcca caggacacct agaatggcca gcagccggtg |
| | ggaaccagga gaggcaagga |
| 58501 | cggatcctct cctccagcct tcagagggag cccggccctg |
| | accacacctc gatctcagac |
| 58561 | tctgacctcc agaactgcga gagaagaaat ctcagttgtt |
| | ttaatgtacg cagcttgtgg |
| 58621 | cactttgtga cagcagatac agcagtactg tgacaacact |
| | aggaaacgaa tacaccatcc |
| 58681 | cagcatctct gctctgtttt ccatacagaa ttgaactaag |
| | gaccaggcaa gctaatgcaa |
| 58741 | Rtccaaacac tgtaaaaact ttggaggcaa tctaaggatg |
| | tgagccagat ttgtttgcca |
| 58801 | gatctttttt attttattta tttatttatt ttattttatt |
| | tttttttttt agtgagaggg |
| 58861 | agtctcactc tgttgcctag gctggagtgc agtgatatga |
| | tctcggctca cagcaacctc |
| 58921 | cacctcccag gtttaagtga ttctcctgcc tcagcttcct |
| | gagtagctgg gattataggc |
| 58981 | acctgccccc atgcccagct agtttttgta tttttagtac |
| | agacaggatt tcaccatgtt |
| 59041 | ggccaggctg gtcttgaact cctgacctca ggtgatccac |
| | ctgccttggc ctcccaaaat |
| 59101 | gctaggatta caggcatgag ccaccaacac ccagcccata |
| | tattcatatt tttaatggac |
| 59161 | ttatacatga aaatggtatc tcacttttaa aaataaatga |
| | tatggaatta tgcgttaaag |
| 59221 | gRaaattaaa tcttattata tcatgtttta aaatttttaa |
| | tttttcatag ctaatacatt |
| 59281 | tatgtagttt taaaaatgta aaacatttgg aaagatataa |
| | tgaaaaataa gtctccttcc |
| 59341 | atctcctgtc tcccagccac ccaattgccc tccccgaaga |
| | caaccaatgt tatcaggttc |
| 59401 | ttgtgtctcc ttccaaagac agtctaagtg tgtttttaca |
| | aacatggata tatacatata |
| 59461 | tatatacacg tatatatgtg tatatatata tacacacaca |
| | cacacacaca catatacaca |
| 59521 | tatatagaca cacacacaca tgtgtacttt tcccttctca |
| | caaatggcag tgtactatat |
| 59581 | gtagtgttct ctaacttggg tttttttttt ttaaacttag |
| | tgcaatttgg aaatctttct |
| 59641 | acatcaaaat tttaaaatct gccctttccc tctttctttt |
| | taacagctgc ctagtatttc |
| 59701 | accgtatgac tgctctgtaa ttgatctaac agttgtagaa |
| | cacttaggtt atttcctgtc |
| 59761 | ttttgctatt tcaacagtgc tgtaatgaat atccttggtc |
| | aggcatcatt tttcatacgt |
| 59821 | gggagtgaat ctttaagaaa accaggagtg gatttgcaag |
| | gtcaagggga atatgcagtt |
| 59881 | tgaacttgga taaataaggc aaattatccc cccttaaatg |
| | ttgtactaat ttttgctccc |
| 59941 | accagctgga gggggagggg ccaaatttcc atatttgcaa |
| | atctgggaga cgaacaatgg |
| 60001 | tgtgtttttt atgcctctta ttacgaatga gtttgaacat |
| | cttttcaaat atttaagagt |
| 60061 | cacctgtagc tcattttcca taaactgtca gttcatatcc |
| | tttgcccact tttttattgg |
| 60121 | cttttggtct ttttcctgtt gagttgtaaa agcacttttc |
| | atgttaaggg aatttgctct |
| 60181 | ttgtctatta tatggttata ctgtcattta aaatggtggc |
| | atagttgctt atagaatgtc |
| 60241 | tgaaccatat gcgtcattgt tagatattta tattgggtct |
| | cattttcttg tattatatat |
| 60301 | gggatgtttt ccctgttttt tgtaaaaaaa aaaaaaaaag |
| | caagatatga cattttaatt |
| 60361 | cttccaatag ttctcaccat aatggtgaag gaaaggaaat |
| | acgtttggga aaatacttcg |
| 60421 | ataggatttg gggtggctgg aacctgtgtg caaaatggcc |
| | aacagggacc aggaccattt |
| 60481 | tgggtgggaa gagagtgctt tgcatggtgt atgtgtatgg |
| | ggcggagggg ctgtatttgc |
| 60541 | tcctgaaaga aaagggggtg gagggagggg agaagaaagg |
| | aaggagagaa ggtggaggga |
| 60601 | ggttagccac agagggagat gactcccaga aacttcttag |
| | gggaaggatg gcaaaagggc |
| 60661 | attttcggtg agtccaggaa tgccctctgc tccgtgtgag |
| | gcaggtacca gcactgcaga |
| 60721 | caccggctct ggctcaccac ctgccatcat gagtgcattc |
| | agcttctcca Ytgagcttcc |
| 60781 | acacaccagg ccctgctcta ggcactgggc tacagccggg |
| | aacacaacca actccctgcc |
| 60841 | ttgtggagct gatgaggcag tagaaacaga caaacaagta |
| | aacaaagatg tgcatgtcat |
| 60901 | atgaggtcag gtggaagtga ctgcagtgaa gaaggcaagg |
| | caggcgaggg tggagcatgc |
| 60961 | tRgatagaga ggcagatgta gccttcttgt agggatgttg |
| | ggacatttga gagagaactt |
| 61021 | gaagggagcg tggcagggaa ccatgggcca tggcatctgg |
| | aggaagaggg aaccagtcag |
| 61081 | aggggatggc aagaaacaaa aaagtgggga gaagaagaaa |
| | gcagagaaga ggagaggagg |
| 61141 | gagaagagga gaaaatgctg cttaaagtct gcttttgtgc |
| | tcccagatgc tcattgtgaa |
| 61201 | tgacattttt gcaatttttg caattttgtt ttgtaattta |
| | ataatgggct gggctgggcg |
| 61261 | cagtggctca cgcctgtaat tccagcactt tgggaggctg |
| | agatgggcag atcacttgag |
| 61321 | gtctgcagtt tgagaccagc ctggccaaca tggagaagcc |
| | ccgtctctcc taaaaataca |
| 61381 | aaaatagcca ggtgtggtgg cacgtgactg taatctcagc |
| | tactctggag gctgaggcag |
| 61441 | gagaatcgct tgaacctgag aggtggaggt tgtagtgagt |
| | tgagatcgtg ccatgctctc |
| 61501 | cagcctgagt tacagaggga gactcggtct caaaaaaagc |
| | aacaaactgc ttgaattaaa |
| 61561 | gcaggagccc tcagctagag gagtgcccac ccacctcctc |
| | cccaggaatc gcagagctcc |
| 61621 | caggagaggc catagcctcc tataggtcat ggcattgcag |
| | gtgggttggt gtcccagctg |
| 61681 | gctgtttctg tctcaagaaa ggaagggctg ctcccgctga |
| | catccaggcg taagcaacgc |
| 61741 | cctggtgaac atcctcgtag ctagagcttt gctcacattc |
| | tggatggctt ccttaggctc |
| 61801 | aatcctcaaa gtggactcct aaagggatat atgaactttt |
| | attttttaaa tgtttgaaac |
| 61861 | cgtgttgtca aatttctctc ctaaaagatt gtgccaatgt |
| | agagaggctg ccgaccgctg |
| 61921 | gaagcttccc aatcccccag cacctatccc ttgtgggtgg |
| | gatggatcct gtaccatcgc |
| 61981 | cagaccctca agaacccctt gtcctgcaYg gtcccttctg |
| | tccctcctgg tgaaatggaa |
| 62041 | ggagcatgca tgccttggga agctggtgct ttgaggcatt |
| | cagatcttac cgtggcatca |
| 62101 | tgtttgaaga gagaaatagc ttgctgtttt gtgtgtctct |
| | caatagtctt attcactgag |
| 62161 | ttcttgaagg tgtcctgtgt gtttctggac tgcagaatcc |
| | ttttattgaa atggacaggt |
| 62221 | cagtgttttc ccagagaaag acctatccag taggagttca |
| | caagaggatt ttaggaggta |
| 62281 | tgtggagagg gcatccgatt ccattgaatc gtaaagtgac |
| | aaatttgttc ccttttcagt |
| 62341 | tctttttcat tcttccagac tagtgaaaga gaaagcctca |
| | gtcaggtgct aacgcatctg |
| 62401 | taaacctctc acactcttga tattttcagc aaggggaggc |
| | caggcctccg gtcagagccc |
| 62461 | tgggtctgca gcaataccca gctacagttc aacagcatca |
| | tagtgttttt tgttttcctt |
| 62521 | ggctttcttt ctttcttttt ttttctagac aaggtctcac |
| | tctgtcaccc aggctggagt |
| 62581 | gcactggttg cacagctccg tgcatagttt cctgcagcct |
| | ctatttccca ggctcaatcg |
| 62641 | atcctcctgc ctcagcctcc caagtagatg ggactacagg |
| | tgcgcaccac cacacctggc |
| 62701 | taattgtttt ttgtttttat ttttgataga gacgaggtct |
| | cattacgttg cccaggctga |
| 62761 | tctcaaactc ctgagctcaa gcagttttcc tgccttagcc |
| | tcccaaagtg ctgggattac |
| 62821 | aggtgtgagc ctccatgccc cacccttgag tttcttttcg |
| | atgatacctt cagttcgtgg |
| 62881 | caagtggtac ttcaaaaaat aaagttaaaa gtgacatggt |
| | ttctagaaaa gctttaagga |
| 62941 | aatggtgttc tggctttcag ataagtcaaa aaaaatctca |
| | aacatgagta aagagttttg |
| 63001 | gaaattatac acttacccct agagggtgag tttgtggggg |
| | aaatttcata atgtatactt |
| 63061 | ccagaatatt tagaactttc caaatgagta caggttatgt |
| | tcgtaaacag gaaaaataat |
| 63121 | aaagacataa taaaatgata atttagtttt aaaagacgac |
| | catttgttga aagcagtatt |
| 63181 | atatttcagt taggaaatgg cctctggaat cagcatagat |
| | tttattgttt tatttatttt |
| 63241 | gtttttgaga cagtgtctca ctctgttgcc cacactggag |
| | tgcagtagtg tgatcacagc |
| 63301 | ccactgcagc ctcaacttcc tgggctcagg tgatccttct |
| | gcctcagcct cccaaatagc |
| 63361 | tgggactata ggtgcacacc actatgccag gctaattttt |
| | ttgcattttt tgtaaagaca |
| 63421 | gattttcacc atgttgccct ggctggtctc gaacttccag |
| | gttcaagcaa tctgcctgtc |
| 63481 | tcggcctccc aaagtgttgg aattacaggt gtgagccact |
| | gtgcccggcc agcatagatt |
| 63541 | ttaaatccac cttttgttgc ttgacttctt ggagcctgtt |
| | tcttcatcta tgaaatgcag |
| 63601 | gaaacgtgcc tggaaatatt gcttgagatt ctaatgggga |
| | ggtgcaggtg gaatactgta |
| 63661 | gcctgatgcc cagcctatgc cgggaactca gtacataaca |
| | gccaatgagc attggctgcg |
| 63721 | gactggacat gtcccctcct tgtgctgtag tattaatgtc |
| | tccaaccctc acagtgatcc |
| 63781 | tgtgaagtaa atgtaaaatc accacccaga tgaggaaaac |
| | agtggcacag agaggttaag |
| 63841 | aaaactgtcc aaggttgcac agcactaaaa tgttgaaatc |
| | aggatttaaa cccagccctc |
| 63901 | ctgaccccag aggatctttt ttcaaaaatt atgttctttt |
| | attttatttc gagatggggg |
| 63961 | tctttcacag tcacccaggc tggagtgcag tcacaatatc |
| | acggcttact gcgcctcaac |
| 64021 | ctcccaggct caagtgatcc tcccacctct gtcccccaac |
| | ccccagttgc ttaggagcac |
| 64081 | aggcacacga caccatgcct ggctaatttt tgtacttttt |
| | gtagagatgg ggtttctcca |
| 64141 | tgttgcccag gctggtctca aactactggg ctcaagcgat |
| | cctcctgctt cagcctccca |
| 64201 | aagtactggg atcacaggtg tgagccacta tgcccagcat |
| | tagaggctct tttaacagct |
| 64261 | ccacaattcc ctgtgagatg ggctgcatac aaagcattac |
| | tcctgcttga catatacaca |
| 64321 | aaccatggcc caaagggagc tgggacttgt ccggatgtca |
| | gcctctaagt cagttttggg |
| 64381 | tcagtgagac ccaaaaccaa ggctctagcc gtctagcccc |
| | tccaaccttt cttctgcccc |
| 64441 | agttttatgg gtttggcaat gtctgccagg atgggaggga |
| | gcagttcaga tgatccagtc |
| 64501 | tggaggcaaa attctgctaa attcaaaggg cccagcctca |
| | agtcactgat gtcttttttt |
| 64561 | tttttttttt actttaaaaa aatgtattMa ttctaaacca |
| | cacaagtaag acacgaatca |
| 64621 | gtcttccttg cagaaagtta aactttttat acataaagct |
| | gatatctatt ttactcactg |
| 64681 | cttccagccc catgcctytc ccctctcctg cagtaacccc |
| | tatttgatat gtttccagaa |
| 64741 | ctttatctaa accttcacat aataaaaata attcttatat |
| | agggcttact aacttccaag |
| 64801 | cactgcttta agagctttac atatttaagc cccacaataa |
| | ctctgtgagg taggtgctgc |
| 64861 | tcttatcccc attttacaga tgagaacatt gaggcccaga |
| | gaggctatgt aactggcctg |
| 64921 | gcgtcaccca gcctgcagtt ggtggaaagg tcagtcccaa |
| | atctaggcag ccttgctccg |
| 64981 | aaattcatgc tcttagcctc taagttataa aacatgctat |
| | tatcgacctg tgggaagaaa |
| 65041 | cagtattgct gtttgttgat gtgtattttg agacaagtga |
| | tatcatgttg tgcatatcat |
| 65101 | tatacaatgt gtcttgttta ttccatgata catcttgaag |
| | atacagccac gtcaacaaaa |
| 65161 | aataagtccc tttcctgtac ataatcccac agcgtggtta |
| | caccaaactt tattatggct |
| 65221 | gtctagggtg tttaaagacc gctgctggaa tttctttatc |
| | caagactctg tgtgtcttag |
| 65281 | cccattgggg ctgctgtaac aaagtgccag agactggctg |
| | gattctaaac aatagaaatt |
| 65341 | tatttctcac agttctggag actaaagtct gagatcagag |
| | tgccagcatg gtcgggttct |
| 65401 | ggcaagagcc cttttccggg ttgccgatgg cggacttttc |
| | attgtatcct cacatggtgc |
| 65461 | aaagcaggct agagcattct cttgggtctc ttttataagg |
| | gcatcaatcc cattcactac |
| 65521 | ggctcctccc tcctgaccta attacctccc ccagacccca |
| | gctcctaata ccctcctatt |
| 65581 | aggggttaga gtttcaacat gtgaatttca ggggagcaca |
| | aatattcagt cgatcacatt |
| 65641 | gtgtattctg ggccccaagg ctgtccattt tgctttctgc |
| | tgagaagttt agtggtaagg |
| 65701 | tcaaagggta gaagcgcagt aagatctaga agtcactgct |
| | gatttgccct acaagatggg |
| 65761 | caaaccactt cccagcctgc agctctgagc cctggatcca |
| | ggccttctct cctttatgtc |
| 65821 | tcgagcttac tctgtcccct gccttactta ccaaaccaaa |
| | gcaagactcc tctgggacat |
| 65881 | tcctgccagg ccccatggca aggttggagc tgaggagggg |
| | acttccaaac cttggtttta |
| 65941 | ttttcacttt ggcttgggtg tgccaagccc tggcagagcc |
| | acagagttgg ggcacgaggt |
| 66001 | gccaagaaac agctcccagt ctgaggaagg tgctctgccc |
| | tgggaagggg ataRagctga |
| 66061 | gtgttggtta tggggagaca tcctcaaggt ctccatctaa |
| | atatggtcaa cttggtggcc |
| 66121 | ttcctgtccc cagggccttt ggRgctctca gaggacaaga |
| | atcactcagc tctgatttgc |
| 66181 | tggagaagca gagccccagg tgggaggtag cgaggagcct |
| | gtctctcccc actgggagtg |
| 66241 | gagagctggg tgccacagcc tcaaacagtt gttcccagac |
| | atcggcacat ggggaacccc |
| 66301 | ccgggagctt ccaaaagtgc cagtgcctgc gttctacctc |
| | ctgggatggt gctgtgatgg |
| 66361 | gtctggagtg tggcccattg ggatttttta aagaccctct |
| | tcccaggtga tgccgtcgtg |
| 66421 | cagacaagca tgggaaccac tggcctggaa gccccaggga |
| | gaggcctctg gggcccggga |
| 66481 | cacgcaagcg ggagccttct cctagcagcc ttgctagcca |
| | cccacccctt cctgtgggtg |
| 66541 | ctagacccaa actgccaggg ctgagcagga gaagcaaggc |
| | caccagctgg ccacctgctg |
| 66601 | tcccagccag gctcaggctg gcagtagggc accccagtcc |
| | tggaggaggg tgcatctgga |
| 66661 | aagcttgggg gctcttggtg gaaagttcaa gcctttagca |
| | taggaaggtg gctggggtga |
| 66721 | aggaggcagt ttaaagacta gtgatgtggg ctgggaccca |
| | gattgctggg tttccaatga |
| 66781 | ccagaagaag atggccttgt tggactgggg acagggacca |
| | cccaggctca ggcctgaggc |
| 66841 | tgggagtgca ggcatgaatt tgagtgcagg ctcaggaaca |
| | tccccagggc cgccaggcag |
| 66901 | ggagacacct cttcaactcc tggctcctcg actctagctg |
| | gtgacctagg caggtccctt |
| 66961 | tcccactgtg agcctcaatt tcctcatgtg taaagcatca |
| | tgactgtgtg ttttaagtcc |
| 67021 | tttagggccc tatctttgtg agggacaaca gagacgatgg |
| | ttttcacagg caacagtcac |
| 67081 | ccctgcatca tcactgactc acagaaatgg atatgcccta |
| | taaggacatc tcttgcttgc |
| 67141 | cctgccagca tttgtgggcg ggggaggaaa tggccactaa |
| | ccattcctga aggcacctcc |
| 67201 | caagggaccc ctgaaaggag ggtgacgcct cccagaaaga |
| | gaccagtggc ccagcccagc |
| 67261 | cctgctctta gttttgtttc ctctgctttg aatgccagca |
| | aggcctttcc ctcccagggc |
| 67321 | ctcagtttcc ccatctttac aatgggcaca gcatagaccc |
| | catcactggg gctctttctg |
| 67381 | ctctgccacc ctgtgcatcc aggctcaggt gttatccgaa |
| | gccctgacat catcagccat |
| 67441 | tcccgggggg aaggcagagg cgctgggttc tctgtgtccc |
| | cctggtaggg cttggtgtag |
| 67501 | gattcaggga ggattggagg gcctctgtgg cctcaggtcc |
| | tcccccaggg tccccaggcc |
| 67561 | ctgacctgtc cacctggacc taaagaacca acaacagcag |
| | cactttctgt ttctttgagg |
| 67621 | tatttctgtc caaatgtctg tagtcatgac tggggtgcct |
| | ggctatcacc ccacaaactg |
| 67681 | catcctttct gatctgcaca gatgagaaaa ctgaggtctt |
| | gtttcccctc catcccaaga |
| 67741 | cctaatggga cctcctcctt aatctaatcc tcaagaggca |
| | gaaacaggat ggaaagcaaa |
| 67801 | ggaatggcag ccagaagtct gYggctccac tcctgtcctc |
| | tctgcatccc tcacttcctt |
| 67861 | ccttacacca ggcgtcaggg gctggagaga cggaagaagc |
| | gatagcaacg acaaccacac |
| 67921 | tagtggtaat catagcttgt gtggattgaa cctttttttt |
| | atttttcatt tttaattttt |
| 67981 | gtgggtacat agtaggtgta tatatttatg ggttacatga |
| | gatgttttga tataggcgtc |
| 68041 | cagtgtgtaa tcacatcagg gtaaatgggg tttccatcac |
| | ctcaagcatt tatcctttgt |
| 68101 | gttgcaaaca attcaattat actttttgtt atttttaaat |
| | gtacagttaa attattactg |
| 68161 | actatagtta ccctcttgtg ctattgaata ctaggtttta |
| | ttcattctaa ctattttttg |
| 68221 | tacccactaa ccatccccac ctcccccacc ctggcctcct |
| | actatacttc ccatcctctg |
| 68281 | gtaaccatcc ttctactctc tatctccatg acttaaattg |
| | ttttgatttt tagatcccac |
| 68341 | aaataagtga gaacaagtga tgtgtgtctt tctgtacctg |
| | gcttatttca cttaacataa |
| 68401 | tggcctccag tgccatccat gtccttgcaa atgatgggat |
| | ctcattcttt ttgatggctg |
| 68461 | aatagtactc cattttgtat gtgtgccaca ttttctttta |
| | ctcttttatt cttttctttc |
| 68521 | tttcgagatg gactcttgct ctgtcaccca ggctggagtg |
| | cagtggcacg atcttggctc |
| 68581 | actgcaacct ctgcttccca ggttcaagca attctcctgc |
| | ctcagcctcc caagtagctg |
| 68641 | ggattacagg catgccccac catgccctgt taatttttgt |
| | acttttagta gaggaggggt |
| 68701 | tttgccatgt tagccaggtt ggtctccaat gcctgacctc |
| | aggtgatcca cctgcctcag |
| 68761 | cctcccacag tgctggaatt acaggcgtga gctgccgtgc |
| | ccagstgcca cattttctct |
| 68821 | gttcattcat ctgtcgatga acacttagat taattccaaa |
| | tcttggctat tgtaaacagt |
| 68881 | gctgtcataa acatagtagt gcagatatct ctttgagaca |
| | ctgattcctt tcttttgggt |
| 68941 | atatacctag cagtgggatt gctggatcat atggtagctc |
| | taaatttagt ttagtttttt |
| 69001 | tttgtttttt tttttttttt gagacagagt ctcactctgt |
| | ctcaaggctg gagtgcagtg |
| 69061 | cgtgatctca gttcactgca acctctgcct cctgggttca |
| | agcgattctc ctgcctcagc |
| 69121 | ctcccaagta gctgggacta caggcatggg gcgccactac |
| | gcccagctaa tttttgtatt |
| 69181 | tttagtagag acagggtttc accatgttgg cgaggatggt |
| | ctcaatctct tgacctcatg |
| 69241 | atctgcccac ctcggcctcc caaagtgctg ggattatagg |
| | tgtgagcctc cgtgcccagc |
| 69301 | cctattttta gttttttgag gtacctccga actgtcctgt |
| | agtggctgta ctaatttaca |
| 69361 | ttcccaccag tgtatgaggt ctcccttttc tccacataga |
| | ttgaggtgtt tgtttgtttg |
| 69421 | tttgtttgtt tgtttggaga tgaagtctcg ctctgtcgcc |
| | cagactggag tgcagtggcg |
| 69481 | tgatctcagc tcactgccgc ctccgtctct tgggttcaag |
| | ctattctccc acctcagcct |
| 69541 | gggattacag gcgcctgcca ccacgcctgg ctaatttttt |
| | ttattttaag tagagacgag |
| 69601 | gtttcctcat gttgcccagg ctgctctcaa actccggacc |
| | tcaggtgatc tgcctgcctc |
| 69661 | agccttccaa agtgctggga ttacaggctt gagccaccat |
| | gcccagcctg gattgagttt |
| 69721 | gtatcatgtg ccaggttcca tgtcagggat tccacatgcc |
| | ccatcccaca accatcttgc |
| 69781 | caggtgggca ttatgatgac cccccatagc agacactgtc |
| | agagctcccc caaccccatc |
| 69841 | tctgcccgga tgcccccact cactgtgggt gacacctgca |
| | tccttcttcc ggggtcgcct |
| 69901 | ttgcctggtg ggcattgcct tgctcagaga tgcctgggca |
| | gtcatgcctc cctcccccgc |
| 69961 | ccaatggggg tgcagtgacg tgaagtgtga ccgaccaccc |
| | agaccccttg cacatgcgca |
| 70021 | ggatggacct cttctatcat cacgctccag ttctcccatg |
| | tggcaggctg aagcRaggct |
| 70081 | tttgaaacca tatctttgct cagctgcctc tgctacccta |
| | tctttcttcc accaccccct |
| 70141 | caataaacaa cttccacagg aacccccttg ggctctgctt |
| | ctagggacct gacctagaag |
| 70201 | atcctcattt tacagatgag gacgctgagg tacgaagagg |
| | ctgcgtaacc taccagggtc |
| 70261 | acacagcttt ggtgtggagt agggactcaa atccagggcc |
| | ctggtacgga ggcatttgcc |
| 70321 | ctcaccaaga ttcaggcact gagagaagcY gacgggccta |
| | gagaatggca agacttgaga |
| 70381 | tagcagaagg aaggagagag ggtgagaaat ggatccccgt |
| | gctgaggtct ggtctggaag |
| 70441 | gagaagtctg caaggtagga aaaagaggaa aggggacgtg |
| | aggtagggaa gcaggatgtg |
| 70501 | caaggcgggg acttgtgaaa gagaatgccg gggggtggtg |
| | agcagcatct catgggcagg |
| 70561 | tcagcaggtg caggagaggc ctaggagggc cggggactgg |
| | atgatgaaag ggcatttgtt |
| 70621 | tatttatcct ttgtttttgt tttttgttgt ttaaattgag |
| | accagctctc acaatgttgc |
| 70681 | ccaggctcgt ctcgccaact gtctgcccac tcctggcctc |
| | aaacgatctt cctgcctcag |
| 70741 | cctccaaaag tgttgggatt acaggcataa gccaatgttt |
| | attcgtcctg agccttccct |
| 70801 | ggacacctga tatgtagctg ctttgtggtg gtcacagttc |
| | catggtgtga ctttatcctg |
| 70861 | gaggcagtgg gatgctgata gcaagttcag acaggcatga |
| | ggccagacca gagctgagtt |
| 70921 | ttcaagataa ctccagccag aggtatgcgt gcatgtgcac |
| | atgtgggtac atgtatgtgt |
| 70981 | gtgcacgtgc atgtgtgtat gagtgtggac tagagatgga |
| | atggctttct gagcaaaggg |
| 71041 | aacagccaag gcaaaggcct gtgggctgca gggagagaac |
| | aaggtttgat ctctgcactc |
| 71101 | cctcctctaa gctctgtgtc aacagcattt tcaggacagc |
| | aggaaggatt cccaaagagc |
| 71161 | acagcagcga gggcagttgg agggaggcgt ctgctgggac |
| | actgaaacca gggYggggta |
| 71221 | aactcccctc gcacctcctc agactcagag cccccataaa |
| | tcttccaggt gcaccccagc |
| 71281 | ccttttccct ctctctgtct gaactggggt ggcatgatcc |
| | caggactgca actccacgtg |
| 71341 | tctggattcc ccttactgac tgcgtgaccc tggccatttt |
| | ccctccacct ttctctcagc |
| 71401 | gcccccattt gattactcgg cctcaggggt gcactgggga |
| | ttcttagagg taacacttcg |
| 71461 | aacggaaagc cagaggtcct ggtgccaaaa tgcaaatgtg |
| | agggcttgaa agttcatcgg |
| 71521 | gttttatttt ctctgttccg tttgcagtag ctcttctttc |
| | ttgttttcta tagaacagta |
| 71581 | aaaaacttta tgcatttgaa tatcgtgaac cttgtaattc |
| | atatgcaata taaaaactac |
| 71641 | tagctatgca tggagaaatc aattgaagaa cagtggctta |
| | gcatgtcttt ctcattcttt |
| 71701 | gatggctacc tcaaatcaaa tcaatatgaa cttggggtca |
| | ggcatggtgg ctcacacctg |
| 71761 | taatcccaac acttggggag gccaaggcag gaggatcact |
| | tgagcgcagg aatttgagac |
| 71821 | tagcttgggc aacatagcgg gacctcatct taatattaaa |
| | aaaaaaaaaa agagaaagaa |
| 71881 | aaaaagtgaa cttgggaggt ttgaatgaca actcgtatag |
| | ctgaagtaat agatatattc |
| 71941 | aacttttaaa acttaatttt cattagataa cacatgcata |
| | tggtataaca ttctaggcca |
| 72001 | ggtgcagtgg ctcacatctg taatcccagc actttgggag |
| | gccaaggtgg gcagattgct |
| 72061 | tgagcccagg agttgaagtc tagcttgggc aagatggcaa |
| | aaccccatct ctgcaaaaaa |
| 72121 | cacaaaaaat tagctaggtg tggtggtgtg cacttgtagt |
| | cccagctact caggaagctg |
| 72181 | aggtcgggga atcatataag cccaggaggt ggagactgca |
| | gtgagccacg accacaccac |
| 72241 | tgcactccag cctgggtgac agagcaatac cctgtctcaa |
| | aaaaaaaagg aaaaaaaatt |
| 72301 | cctttcactg gggacaatta cctttatgaa tttcttccag |
| | aaatattaat gtatttgtat |
| 72361 | acacattttt ttttgagaca gggtctcact ctgtcaccca |
| | ggctggagtg cagggatgca |
| 72421 | atcttggctc actgcagcct ggataccccc aggctcaagc |
| | aatcctccca cctcaacctt |
| 72481 | gcgggtagct tggacaacat gtgtgcacca ccactcctgg |
| | ctaatttttg tattttttgt |
| 72541 | caagatgggg ttttgccatg ttacccaggc tcgtctcaaa |
| | ctcctgacct caagtgatct |
| 72601 | gcccgccttt gcccctcaaa gtgctgggat tacaggcatg |
| | agccaccata tttatttttt |
| 72661 | ctttacacaa aggaagcata ttctacaccc tgttttgccc |
| | cggctctctt ttaaaaaaca |
| 72721 | gtgtatcttg gataccattc catatcattc cataaagttc |
| | atcttcatcc tctttttttt |
| 72781 | tttttttttt tttttgagac agggtttcac tctgtcgccc |
| | aggctggggt gcagtggcac |
| 72841 | tatctcagct tacctcaacc tcccgggctc aagtgatcct |
| | cccacctcag cctcctgagt |
| 72901 | ggctggatgc tgcttctttc tttttgatgg ctgcaaagaa |
| | ttccattgta tagatgtgcc |
| 72961 | ataatttaat cataatatgt cctctttgat catttaggtt |
| | tttccaatct tttgctatta |
| 73021 | caagcaaggc cacgttaaat atccctgggc atattatttt |
| | gcacattatg aagatatctg |
| 73081 | taggctaaat acctgtgtat ggaattgctc agtgtgatta |
| | catggcattt accactttgg |
| 73141 | tagataaagg ttgtatcctg ttacatgtcc acggatgaat |
| | ttcttccaga aatattcatg |
| 73201 | tatttgtata cacatttttt gagacagggt cacaaaaccg |
| | ttaacacatt attattatta |
| 73261 | ttattattat tattatttca cattatttag ggaagtttca |
| | tttttttgtg agctatcact |
| 73321 | gataatttct tatttactca tcatcttgta tttctcttcc |
| | ttgcacattg gggaaatgtc |
| 73381 | tattctagtt tttaatgtaa atgttcttgt ggctttttta |
| | gaaaaaatca aaatgtgcac |
| 73441 | attaaaaagt ttgaatggtt caaaaggtcc taaaaagggt |
| | ccacagtgga aaaggctctc |
| 73501 | ctttcccagc aggcccagat ccctcctcag acacaagcct |
| | gtcgtcagtt tcctgtgtag |
| 73561 | cttcccagag ttggtcacta tgtatgcaaa tacacacaca |
| | tttaaaatat acacactcca |
| 73621 | cgtccacgtg gaatgatgca aagcatctaa gtgcccgtta |
| | attcacaacc ttgccaactc |
| 73681 | agtaatatcc ttgcgaatct aatagctaga aaatggtgta |
| | tctttttagt tctaatgtat |
| 73741 | tatatttatt ctattttctg tgagcttgaa catcttttca |
| | tatatttaaa agtcattggg |
| 73801 | gccaggggtg gtggctcatg cctgtaaccc cagcactttg |
| | ggaggccgag gcaggtagat |
| 73861 | cacctgaggt cagaagttcg agaccagcct ggtcaacatg |
| | gtgaaactca gtctctatta |
| 73921 | aaaatacaaa aaaaaaaaat taatcaggca tggtggctcg |
| | tgcctgtaat cccagctatt |
| 73981 | gggaggctga ggcagaagaa ttgcttgaac tggggaggtg |
| | gaagttgcag tgagtggaga |
| 74041 | tcgtgccact gtactccagc ctgggcaaca gagtgagact |
| | ctgtctcaaa aaaaaatttt |
| 74101 | taaatgtcat tggtgtttcc cttttttgta aaccttttat |
| | gttctttgcc tagttttcta |
| 74161 | tgaggtcatt ggctttttca gtgttggttt ttagtaactc |
| | tcgatttatt aaaacattta |
| 74221 | tacatttata gctttgttta tgatgtatac atttataacc |
| | ttgtctgtga tgcagatatt |
| 74281 | tttccaattt gtcatctggc ttttgacctt atctgtggta |
| | ttctttgaca atacaaaaat |
| 74341 | tttaatttat atctgatcag tttcatccag ttgttttttt |
| | cttttttcat gatttctgag |
| 74401 | ttttgtgtca tgcttagcaa gatgctctct actctgaaat |
| | tattttctgt aaaatatttt |
| 74461 | tttcagccag gcatagtggc tcacacctgc aatcccagaa |
| | ctttgggagg tagaggtgga |
| 74521 | aggattattt gagtccagga gttcaaggcc agcctgggca |
| | acagagcaag accccgcctc |
| 74581 | tattttttaa aagagaaatt tttttaaaat actttttccc |
| | atgttttgtt tgagcactta |
| 74641 | cattttctta acttttttgt cgtgaaacat aacacataga |
| | aaaatgtaat gtaagtgtac |
| 74701 | aacttcatga attgttataa attgaacata tagctttctg |
| | gtactttcat ggtttaactt |
| 74761 | cttacattta aattttggtt tcatctagac tttattttga |
| | tataattgtg aaatagaaat |
| 74821 | ccatttttat tctaaaagaa cactttttaa atgttcatga |
| | aatgttttaa aatttattat |
| 74881 | ataccaagca agaaaaaatc tcaacatagt cctcaaagca |
| | gaaattgcac aggacttatt |
| 74941 | ctttgtacag tgtaatgcac aaaaactaat aagaaagcct |
| | taaaaacaac cctttattaa |
| 75001 | attactccat tccaaattat aatttcaatc tacttagcac |
| | ataagtattg atatacattt |
| 75061 | atcgaaattg gccaaaagtg ttttattatt ttttaaaaga |
| | aaaagcctag caatggctta |
| 75121 | agcaataaag tttttaattt taaagaataa caaacagcct |
| | cactaaatca gaggaaagaa |
| 75181 | ataaaaagat aaaagcaaaa attacagaac tagaaaaggg |
| | accattccag caaaattgat |
| 75241 | aaatgaaaac aaaaatttgt ttttcaaaca taagagttac |
| | caacggctgg tgaatctaaa |
| 75301 | aagaagaaaa aacaaagtaa actaaattaa atgtgaagcc |
| | atgaagagat ttaaaattat |
| 75361 | aagaaaatag gccgtgcacg gtggctcacg cctgtaatcc |
| | cagcactttg ggaggccgag |
| 75421 | gtgggtggat catgaggtca ggagcttgag acccgcctgg |
| | ccaacatggt gaaaccccgt |
| 75481 | ctctaccaaa aatacaaaaa ttagctggct gtggtagcat |
| | gagcctgtag tcccagctac |
| 75541 | ttaggaggct gaggcgggag aatcacttga acccaagagg |
| | tggaggttgc agtgagccga |
| 75601 | gaccttgcca ttgctctcca gcctgggtgg cagagtgaga |
| | ttccgtctca aaaaataaat |
| 75661 | aaataaataa agctgggcgc agtggctcat gcctctaatc |
| | acagcacttt gggaggccga |
| 75721 | ggtgggcgga tcacaaggtt aggagatcga gaccatcctg |
| | gctgacatgg tgaaaccccg |
| 75781 | tctctactaa aaatacaaaa aatgagctgg gcatggtggc |
| | gtgtgcctgt ggtcccagct |
| 75841 | actcgggagg ctgaggaaag agaatggcgt gaacctggga |
| | ggtggaggtt gcagtgagct |
| 75901 | gagatcacgc cactgcactc cagcctgggc gacagagtga |
| | gactccattt caaaaaaaaa |
| 75961 | aatacaagaa catattatat acaactgtga gctacaccat |
| | gtttgaacat cttgacaaaa |
| 76021 | tgagcaactt tctcaaaact aaattgccaa atgttcattt |
| | ttaaaaagcc aaaaataacc |
| 76081 | aaccaacaca atgaggataa atttaaagtg atcaagtttg |
| | atttttttaa aaaactgttc |
| 76141 | aaacatcttg gtagtgctgg gcaggaagtt ggctttcatt |
| | ctgggtgaga tgagaaggct |
| 76201 | ctaaggggca taggacctga tgtcatggga cctgatgcag |
| | gctctgtctt aatagctgac |
| 76261 | tcctgcgtgg agtaggttat tgggggatgg gtggacagag |
| | accagggagg aggctggtgc |
| 76321 | aggaatccca gtgagaaatg atggtgtcca ggctaggcca |
| | tagcagtaga ggaagcaaga |
| 76381 | ttctggaatg cacttggaaa atgaagccat ctttgggctt |
| | gagctcaagg aagcatgaag |
| 76441 | ctgcccctaa ctgaagtggg gaaggtggtc tggggagcag |
| | tttaggagga gagatcagga |
| 76501 | gctcagtttc ggacttggaa attttgaaac gcacatgagg |
| | catccaggtg gagttgggag |
| 76561 | aattaaataa tgctgaaagg tactcacttg gcaagtagca |
| | aatattcgct atattgtctg |
| 76621 | ttcaactttg tcaactgctt ttccagtaga aatgtagtgt |
| | ccttatcaca catgcataaa |
| 76681 | tgttaaactc aaaagtaaaa tgaaatattt tctcagtaga |
| | tatgaataac tttttaattt |
| 76741 | ttttgttttt tgacatgcgg tctcactctg ttgcccaggc |
| | tggagtgggg tggcgagatc |
| 76801 | atagctcact gcagcctcaa actcctgggc tcaagcaatc |
| | ctctgccaca gcctcccgat |
| 76861 | tagctgggac tacaggcaca caccaccacg cccagctaat |
| | cttttatttt ttgtagagac |
| 76921 | tgagttgcac aatgtagccc aagctggtct ccaactcctg |
| | agctcaagct atcctcccac |
| 76981 | ctcggcctcc caaagtgttg ggattacagg cgtgagccac |
| | tgcacctggc tgctttttaa |
| 77041 | ttgtaatatt catcctccat ccatcatact tagtgcaaat |
| | atttttcccc acctattgcc |
| 77101 | ttcctatttc tcttttattt cacgtcattt agggaagttt |
| | catttttttg tgagctatca |
| 77161 | ctgataattt cttatttact catcatcttg tatttctctt |
| | ccttacacat tggggaaatg |
| 77221 | tctattctag tttttaatgt aaatgttctt gtggcttttt |
| | ttaaaaaaaa atcaaaatgt |
| 77281 | gcacattaaa aagtttgaat ggttcaaagg gtcctaaaaa |
| | gggtccacag tgggaaaggc |
| 77341 | tctcctgtcc cagcaggccc agacccctcc tcagacacaa |
| | gcctgtcatc agtttcctgt |
| 77401 | gtagcttccc agagttggtc tctatgtatg caaatacaca |
| | cacatttaaa atatacacat |
| 77461 | gccacagctt tttaaaattt gaatttatgg gccatgtgca |
| | gtggctcacg cctgtaaccc |
| 77521 | caacactttg ggaggccgag gcaggtggat cacctgaggt |
| | caggagttcg agaccaacct |
| 77581 | gaccaatatg gtgaaacccc gtctctacta aaaatacaaa |
| | aattagctgg gcatggtggt |
| 77641 | gggtgcctat aatcccagct tctcaggagg ttgagacagg |
| | agaatcgctt ggacccagga |
| 77701 | ggcagaggtt gcagtgagct gagatcgtgc cactacactc |
| | aagtctaggc gacagaggga |
| 77761 | gactccgtct caaaaaaaaa aaaaaaaatt gaatttagac |
| | tcaggtgcag aggctcacgc |
| 77821 | ctgtaatccc agcacttacg gaggctgatg aggggagaat |
| | cacttgagtc cagtagttca |
| 77881 | agaccagcct gggcaacaca gggaaactat gtctctacaa |
| | aaaaattaaa attagcctgg |
| 77941 | catggtggag tgtgcctgtt gtctcaccta ctcaggaggg |
| | tgaggcaggg gaatcacttg |
| 78001 | aactcaggag taggaggctg catgagctat gattgcacca |
| | ctgcactcca gcctgggtga |
| 78061 | cagagcaaga ccctgtgtca aaaaaaaaaa aaaagaaaag |
| | aaaattagac taccatcata |
| 78121 | gatggtacaa acacttccca gtttgttgct ttcgttttgt |
| | ttgttgccat ttttactata |
| 78181 | cagaaaaaaa aggcaataaa gaatacatta ttataatatg |
| | ttaatttcta ctgcattatt |
| 78241 | tttaacaact tcattgaggt gtaatttaca taccgtaaaa |
| | tctatccatt ttaggtgtag |
| 78301 | gattcagtga tttctggtaa atttactgag ttatgtgacc |
| | atcaccacaa tctggttttg |
| 78361 | ggacattttc attactgcca taatattcct caaacccatt |
| | tagaatcatt cctatttctg |
| 78421 | ctcccaggcc caggcaacca gtaatctact tgctgtttca |
| | atagatttgc cttttctgga |
| 78481 | tgttttacat aaatagaatc atacaatacg tgatctttag |
| | tgatcttttt tcacatggca |
| 78541 | taaacttggt tcctttttat gttgaataat attccattgt |
| | atagatatgt cacagtttgt |
| 78601 | ttctccattc atgagctttt gggtatttgg gttgatttca |
| | ctttttgact gttatggata |
| 78661 | atgctggtac aaacattcat atacaagttt ttgtgtagat |
| | gtatattttc atttctcttg |
| 78721 | gaatagacct aggcgtggaa ttgctggatt agatggtaat |
| | tctattgttt tacattctaa |
| 78781 | ggcaggggtc cccaaccccc aggctacaga tcagtgtcgg |
| | ttcatggcct gttaggaact |
| 78841 | gggccgcaca gcaggtggtg agcggccatc gggcgagtat |
| | tactgcctga gctctgcctc |
| 78901 | ctgtcggatc agcggtgcca ttagattctc ataggagcac |
| | aaacccagtt gtgaaatgcg |
| 78961 | catgtgaggg atctaggttg cgagtttctg atgagaatct |
| | aatgataaat gtaatgagct |
| 79021 | tgaatcatcc cgaatccatc ccccaacccc aacccccgtc |
| | cggggaaaaa atgatctctc |
| 79081 | atgaaatcag tccctaggcc aaaaagtctg gggactgctg |
| | ttctaaggaa ctgccaaaac |
| 79141 | tgccaaactg tttttcagag tggctggacc attttacatt |
| | cccaccggca atatatacgg |
| 79201 | tgtcctgttt ctttacatcc tcatcaacat gtgtcttttt |
| | cattacagcc attctatgtg |
| 79261 | ggtgtgaaat gctatgtggc tgtgtttttc atttccatgc |
| | agaaattttt aatttaattg |
| 79321 | cttagttaaa aataattctt taaatttaaa acactcatat |
| | tgccatatgt atatttttat |
| 79381 | actacaaagt gatacaattt ttgttaattt tcttgccttg |
| | attactcttt acctgtatag |
| 79441 | tatttaaatc tttacataag atcacaatta tagtgaatag |
| | taatttattc aactcaaaat |
| 79501 | cttcatagga tttagaaaat ttaagtcctt taccaaaggg |
| | attgacttYt aacttcatta |
| 79561 | aaggttgcga acagtatcaa atccttggta aagccacatt |
| | caatctttgg cactccagga |
| 79621 | ttttgagcta taactgtagt gatcactaat atgtcatttc |
| | taagctgacg gtcataatga |
| 79681 | ctaaaacctc tcatagattt taaaatactt ccttcaaagt |
| | cagtaaacat tccaagttac |
| 79741 | taagctgttg aacaatttct tattttgaag attttttctg |
| | ataagttcca aagtgtttct |
| 79801 | gatgaaggaa ataaaagctg tccagagaaa tctgagtcat |
| | tgaggtgagc acagattcca |
| 79861 | ttggctgctt gtgctttcat cattacagta caattaactt |
| | cagaagtcga gagatgctgc |
| 79921 | agaattttaa gtacccaaag tttctcaaca agttgatttt |
| | caagcaaggt cattaactgg |
| 79981 | gccattgttt ttgccaatcc tccaacttca gccatttgaa |
| | tcttgtatga tgaactcact |
| 80041 | tgctggtaac ctggaatatg ctcctttggt ggttgtgcct |
| | gataaaagtc aacaatacgt |
| 80101 | ttacaatttt ttatcctcaa ttcagaactt ggtattttca |
| | ttaagtcacc cagaagcgca |
| 80161 | actgaactag cagtatctcc agcataagtt atttcatccg |
| | atactttctt tttcaaaaat |
| 80221 | ggcaagccac actattttat gatatcatat gcagattcta |
| | caaaatgcgg ctggttttca |
| 80281 | atttttactg cacacagatt cagaatttca aatgtctgta |
| | ctaaatccct taagggaagt |
| 80341 | tcattctgat aacactgtac cagtttcttg acagatttaa |
| | gttgcttttc ttctaagcct |
| 80401 | tctttagcag tctcttcaaa aagtttgatg acaccattaa |
| | ggtccacagg cttcaaaaca |
| 80461 | ccagttccac atggcgaggg aggccttggg aaacatcatc |
| | atgatggaag gcaaagggga |
| 80521 | agtaaagacc ttcttcatgg tggcaggaaa gagacagcat |
| | attgctatgt attttaacac |
| 80581 | cacttatttc cccccttttt tctttcaata tcatggatta |
| | aatagtgtat tttaaatgag |
| 80641 | tgtgaggctc acttggactc tattccatcc ctttggccaa |
| | tttttttcca tttttaatcc |
| 80701 | aagaatctta aagtttgaat gatsgtcacc ttgttatata |
| | ttttattatg tttggaagct |
| 80761 | tttttgctat tgtctgatcc tcccaacaaa tagccttttg |
| | atagaattgc ctttattcgt |
| 80821 | ttctttatat ctaattctaa cttaagatta gacttaaaag |
| | taacttcagc ggttttatta |
| 80881 | tgaaatattt aaggcatata aatactgcct cttttctcta |
| | aatgactgct aatataattt |
| 80941 | taccaagttc tgttaaagta tcctgttgta cataaagtgc |
| | tgttgttttt tatctgcctg |
| 81001 | ttaacttaga gtgggtttta ttttctttca ctttctgtgg |
| | tcgaaggcaa gtatgtctta |
| 81061 | gtcagtttgg gctgttataa tgaaacactg tagactgggt |
| | ggcttaaaca acagaaactt |
| 81121 | gtttctcacg ttctgaaagc cgggaagtcc ccaatcaggg |
| | agccagcagg cctggtgtct |
| 81181 | ggagaggatg cgcttcctgg tttgcagacg gcaccttctc |
| | attgtgtcct cacagggcag |
| 81241 | agagcaaaaa gaaagggcaa gcgctctcca gcctcttcct |
| | ttttcctttt tttttttgtt |
| 81301 | tgtttgagat ggagtcttgc tctgttgccc agactggagt |
| | gcggtggcat gatcttggct |
| 81361 | caatgcaacc tctgcctcct ggcttcaagt gattctcctg |
| | cctcagcctc ctgagtagct |
| 81421 | gggactacag gtgcctgcca ccatgcctga ctaattttta |
| | tgtttttatt agagatgggg |
| 81481 | tttcatcatg ttggccagac tggttttaga ctcctgacct |
| | caagtgatct acctaccttg |
| 81541 | gcctcccaaa gtgctgggat tacaggcgtg aaccaccgca |
| | cccggcctcc agcctcttct |
| 81601 | tgtgagggca gtaatcccat cataaggggt ccaccctctt |
| | gacctaatca cttcgcaaag |
| 81661 | gtcccacttc caaataccat cacactgggt atttaggctt |
| | caccaggtga attggtgggg |
| 81721 | gaggtgggac acaaacattc agtccacggc aagtttttat |
| | ctcaatttgc taaagcctct |
| 81781 | ctttatttct cctgttacag tgtgataatg ttttttaaaa |
| | tatcatttag ctggctgtgg |
| 81841 | tgacccatgc ctgcagtccc agctactcag gaggctaagg |
| | caaaaggatc acatgaacct |
| 81901 | aggagtttga atccagcctg ggcaacatag aaagaccctg |
| | tctcttaaaa aaataaataa |
| 81961 | ataaataaat aaataaataa ataaataaat aaataaagga |
| | aagaggctgg gcacagtggc |
| 82021 | tcatgcctat aatcccagca ctttgggagg ccgaggcggg |
| | cagatcacct gaagtcggga |
| 82081 | gttcaagacc agcctgacca acatggagaa accccttctc |
| | tactaaaaat acaaaattag |
| 82141 | ccgggtgtag tggcacatgc ctgtaatccc actacttggg |
| | aggctgaggc aagagaattg |
| 82201 | cttgaaccag aaggtggagg ttgtggtgag ccgagatcat |
| | gccattgcac tccagcctgg |
| 82261 | gcaacaagaa Igaaactcca tctcaaaaaa aaaaaaaaaa |
| | aaaaaaagaa agaaaagaaa |
| 82321 | agaagcactt aaagaggctg aggcaggcag atcacttgaa |
| | gtcaggagtt caagaccagc |
| 82381 | ctggccaaca tggtgaaacc tagtctctac taaaaatact |
| | agaattagcc agacgtggtg |
| 82441 | gcacatgcct gtaatcccag ctactcggga ggctgaggca |
| | tgagacttgc ttgaacctag |
| 82501 | gaggtggagg ttgcagtgag ctgagaacat gccattgcac |
| | tccagcctgg gcaacagagc |
| 82561 | aagactccat ctcaagaaaa aagaaagaga gagaaagaga |
| | aatatagaca tatgttatta |
| 82621 | gtcccccagg acctcttcaa aagatctctc ttttcctttc |
| | ttatttctaa ttaccatact |
| 82681 | acataccaca tccattatgt cagaatgtgc cctatttacg |
| | tggatttgtc taaagtcatg |
| 82741 | gaactctgag ttacattccY cgtatttcac agagacacaa |
| | tcagagcaca cgctgatctt |
| 82801 | ccgcctctct tcatttcctc catgtactct gtagctacct |
| | gttctcttct ttgttcgtag |
| 82861 | gaagatctct gttgcgtggt gtgggagttt gtgccagctc |
| | atctttttat tgtgctttct |
| 82921 | gtgagcagct atcaatgcct ccaggtcttc tctttaagaa |
| | gaatgtttac tctcaattgt |
| 82981 | ttaaaaaact aactaaatag gaagaagatt gcaaatccta |
| | acctttttgg ggttttatat |
| 83041 | tgggggcaaa agttggcatc tgcagtaagg attatttcat |
| | tttcatctga ataaatgagc |
| 83101 | agtaataact ttgctgactt gcagggctca ttgtgatgcc |
| | aaaggacatt gtaaggtgca |
| 83161 | tatctgaatt ggaggaggaa gaggggcatc ctctgagcca |
| | ggtgtggcag gaggtgacat |
| 83221 | caaggagggt taggctgcgt aaacccaggg acacaaggag |
| | ctgcagtcgg ggaagaacaa |
| 83281 | ggtctagcgg atggagttgg ggggactgtg tctggagcca |
| | atgttctcag atgtgtctcc |
| 83341 | ggcaggcagg ggcaagggct ggtgcctggt ttacagcatt |
| | ccttttggag tctggaatag |
| 83401 | tggggaaaac tccctgtctt ctctcccctt aggagtctgc |
| | actatggaaa caacctgtca |
| 83461 | atccagctca aggcacacat agcccagaca cccatgagac |
| | cctctccgtg gggaccctag |
| 83521 | agcacctatc atgaacgagg agaccaagta ggtgattttt |
| | ctctcccctt cacctcctct |
| 83581 | tttcccctcc tatctacttc gtcacttttc tcctactgcc |
| | aagactggtc cctacccaaa |
| 83641 | tctgccttac tgggctgccc ccaacacaac aaacctgccc |
| | ttgaccatca ttcagcatga |
| 83701 | gaatgtaatg atcagacaca gaaaatcagg aagagttttg |
| | agacagcatc taatctaccc |
| 83761 | attttagaga tggtatgact gaggcttata taggaaaaag |
| | gcttgctcaa ggtcacacta |
| 83821 | tgagtgtatc agctaggata ggccaggtta cactgtagta |
| | catgcaccct ccaaagtccc |
| 83881 | agtgactcat ggcaacaaaa gtttatttcc tgctcttgct |
| | tcctgtctcg tgtggattga |
| 83941 | ctgggattgc tcctccacat catcctgtct cccacactca |
| | ggctaacagg gtagccatga |
| 84001 | tttgcaatat ttctggtccc tggagtagaa aaaaagaaaa |
| | agtactgtca attacacctt |
| 84061 | ggttcttaaa acaactacag gcatgacatg agtctttttt |
| | agcatgtttc attgaccaaa |
| 84121 | gcaagtttca tggctgtgcc caatttcaag gaggcagaga |
| | agtgtaatcc caccaaaggc |
| 84181 | ctagaaagag gagaaagagt atctgtgaac agccctaatg |
| | cctaccacag agaattagta |
| 84241 | acagagctgg gccatggatc tttcaaccat cgtcctctgt |
| | ctccaagatt attccccatg |
| 84301 | gaaatctcaa tgactgataa tgatgccatg tgctttttct |
| | cctgaggatg gttagcctta |
| 84361 | aagagatttg tgtcatatcc taaggggata gaacactaat |
| | gatggaaaca aaggtattgt |
| 84421 | gttagactgg ctggaggaga aatttgagga tgagaaacat |
| | caccagaaat cacaccatcc |
| 84481 | agycactact tacttccatc tggtgccatg ctgccctgga |
| | ccatttcttc atgggtagca |
| 84541 | tggatgctgg acaccatagc ctggaaaaga agttatgccc |
| | taggctatgg ccacatcctt |
| 84601 | ttgccgagat cacacaaaat ccagggactt agccttgtct |
| | ggtgccatct aggcctccaa |
| 84661 | ggccttggaa atttactggc ctgggaggca agaaacctca |
| | gttctcctcc tgcctctgtc |
| 84721 | atsgacttgc tgtgtgactg tgggcacacc ctttgccctt |
| | ccttggccat ggtctttccg |
| 84781 | tctgcttagt ggaggtggtg atccctgcct gaggatcatg |
| | gtgtgagcat gtttcataaa |
| 84841 | ttctaagact ctctgcctga gtgaggacct attgctatta |
| | gcccagttga ggagaaatga |
| 84901 | ccagtggggc tgacagtaca tccaagtatg tctctactgc |
| | atgcccaggg agcagctggg |
| 84961 | ctctaaaccc caggacgcat agtactggcc tcagcctctg |
| | ggaattggga aggctggctt |
| 85021 | ctgggtggac ctcctactct tttcagagtt agaggtggac |
| | tctctggagt tgtgaggata |
| 85081 | aaataaggta tataaagcac ttagacagtg cctaatacac |
| | agtgagtgct ttccaagtgt |
| 85141 | gtactgctat gaggaaggac atagtaccag aaagtacatc |
| | cggaaatgtg atagcatagt |
| 85201 | ccactagatg atggaccacc atttagttgg ccaatcccca |
| | actgttggac atttaggttg |
| 85261 | tttccaattt ttaataatta taaataatct tgcagtgagc |
| | atctttgcaa ttaaatattg |
| 85321 | tatatacgtt catgatcgtt tgccaaggtt agagtcctag |
| | aaatggaatt actgaatcaa |
| 85381 | aggaactgca cattatttag gcttttgctg tgtaaatgcc |
| | ccgtgccctc tattggtact |
| 85441 | tctagaagga ggtggtttaa gagttccagt tcggccatca |
| | tgatggctca cacctgtaat |
| 85501 | cccagcactt tgggaggccg aggtgggcag atcaccctga |
| | ggtcaggagt ttgagcccag |
| 85561 | cctggccaac atggtgaaac cccatctgta ctaaaaatta |
| | caaaaattag ctgggcattg |
| 85621 | tggtgtgcac ctgtaatccc agctactcgg gaggctgagg |
| | cagaagaatc gcttgaaccc |
| 85681 | gggaggcgga ggctgcagtg agccgagatt gcaccattgc |
| | actccagcct gggtgacaga |
| 85741 | gcgagactct gtctcaaaaa aaaaagagtt ccagttcaga |
| | agggaagaat ttgcttagca |
| 85801 | aaagaggtga tggtctgtgt tagctaggat tagagttcac |
| | tgtgagtagc agaaagcact |
| 85861 | aaacaacagt ggcttaacca gaatggaagt gtatttccct |
| | gccatgtaac agaagtccaa |
| 85921 | agtcagtgag cacagggcag atgggcagct ttaccttcca |
| | ccttcctcag tatgggttcc |
| 85981 | acctcatgat tcaatatggc tgcccaagat ccagccatcc |
| | catcaacaat ccagacaaca |
| 86041 | ggaaggagca aagaagaata gacaacattt ctacttacat |
| | tccactggcc aaattacatg |
| 86101 | gctacatata actgcaaggg agactgggat ataataatct |
| | attctagatg accagagaat |
| 86161 | atttagtagt tctaatactg aggaagaagg gaagaacaga |
| | ttttgggaga caactagttg |
| 86221 | tctctgccat gaggttagct cagactgaga gcagcataaa |
| | tggttcatgg aaataagaat |
| 86281 | gagtaatgca tcctcacggg actgtccaca ggggatgaac |
| | aagaccattc cagagagcca |
| 86341 | gggtgcaggc acaaatggag tagggagggg gtgcagaaga |
| | aacagactga tctctggagc |
| 86401 | tgatccctgg gcagagacat caagactcca tggagaggcc |
| | acttccctgc ctattttatg |
| 86461 | tcKctcaaat gtagggactg gttcttgact gcttgacttc |
| | cctagaaact cccttatccc |
| 86521 | ccaactcacc ttScccagat caccttcccc ctagttcatc |
| | tgtaccccac tcacctgccc |
| 86581 | tcaggtcacc tgccccacaa ctcacctgtt ctccatctaa |
| | tcttcctccc agctcccctg |
| 86641 | tcccccaaat cacctgcccc caatttgctt gtcctccagc |
| | tcaccggttc cccagctcat |
| 86701 | cttccttcca gttcccctgt cccccagctc acctgcctgc |
| | cctcagttca cctgtccccc |
| 86761 | agttcatctt cctcctagct cccctgtccc ctagctcacc |
| | tgtgccccag ctcccctgtc |
| 86821 | ccccagttcc cctgtccccc agctcacctc cccctcagct |
| | ctcctgtccc ccagctcatc |
| 86881 | tgcccccagt ttacctgtcc cctagctcac ctccccccca |
| | gtttacctgt cccccagctc |
| 86941 | acctcccccc agtttgcctg tcccccagct caactccccc |
| | caagttcccc tgtccctcag |
| 87001 | ctcatctgcc cccagtttRc ctgtccccct caactcacct |
| | atcttccctg tccctccagg |
| 87061 | gctggctcct catggacccc gttggcctcc agctcggcaa |
| | caagaacctg tggagctgtc |
| 87121 | ttgtgaggct gctcaccaaa gacccagaat ggctgaacgc |
| | caagatgaag ttcttcctcc |
| 87181 | ccaacacgga cctggattcc aggaacgaga ccttggaccc |
| | tgaacagaga gtcatcctgc |
| 87241 | aactcaacaa gctgcatgtc cagggttcgg acacctggca |
| | gtctttcatt cattgtgtgt |
| 87301 | gcatgcagct ggaggtgcct ctggacctgg aggtgctgct |
| | gctgagtact tttggctatg |
| 87361 | atgatggtaa gggcaggtgt gagagacgca aagcagccgg |
| | gccagtacct gcgtcatggg |
| 87421 | gagccgggag gagcccaggg acccacctaa gtcagagaga |
| | gggtgtagcc cagccacgat |
| 87481 | ctcaactcta tatttctgga cacttcccag aacaaatttc |
| | ctaatgcgcc caaatctgct |
| 87541 | ggggcctgga ggacccctcc ttctacctgg cttcactcga |
| | attgctcgaa ttgctgagtg |
| 87601 | actgcttcca gccacaaaat cccacatagc ccataatgcc |
| | cgagagattt atttcttctt |
| 87661 | ttttggcggg ggtggggggg tgttttttgt ttgttgttgc |
| | tcttgttggg ttttgtttgt |
| 87721 | ttgtttttta tttttgagac aaggtcttgc tctgttgcct |
| | aggctggagt gcagtggcac |
| 87781 | aatcatagct cactacagca tccaactcca ggactcaggt |
| | gatccttccc acttcagcct |
| 87841 | cccaagtagc tgagactaca gtagaactat aggtgcatgc |
| | caccacatta ggctaatttt |
| 87901 | tcattctttt gtagagacag gacatcactg tgttgcccag |
| | gctggtcttg aactcctggc |
| 87961 | cttaagtgat cctcctccct tggcctccca aagtgctggg |
| | attatagatg tgagccatca |
| 88021 | cacctggcta gaggtgtttt tgtttgtttg tttgttttgc |
| | aacatggtca gaaacagtga |
| 88081 | cggggagcgc cttctgccct ctactgatcc gtggggattt |
| | cttgggcacc ttctgtttgc |
| 88141 | acagcactga ggggaaacac aggacattga ggggcaacag |
| | cgtttttgga gagaaaatgg |
| 88201 | gaactcactt aagaacccta cttcaaggca gagtgtggga |
| | attgcggggg gcggggcggc |
| 88261 | gcatgcacat atgtgcagac atatgcagat acacacagtg |
| | catatacatg tgggtatgtg |
| 88321 | cgtaaaccca tgtacacatg catgcaagtg tgcccacatg |
| | cataacacat gcatgcctgt |
| 88381 | gcacacacat acataaactt gcatgcactt gcatatacac |
| | acatttctgc ccagcactgc |
| 88441 | cttcaaagct caccaccaca ttttctccac aacagataac |
| | tgagtgttcc atttagttct |
| 88501 | cccttgctgg gtatctccct gtgagatgag ccctgagcca |
| | ggcgccaggc cagagctggt |
| 88561 | ccccagcatc cctcgacagc cccataccac attatactct |
| | cccctctctt gcagggttca |
| 88621 | ccagccagct gggagctgag gggaaaagcc aacctgaatc |
| | tcagctccac catggtgagg |
| 88681 | actggagttg gggggtggga agggggtggt gagcactgtg |
| | aagtccccac ttccaagctg |
| 88741 | gaggaggctg tggaggctgt ggtggctggt cacagccatt |
| | tctcataggc catgctcttc |
| 88801 | gtctgtccct gacccatttg caagtgtgtc aagcccctaa |
| | ctgtgtctcc cagccccacc |
| 88861 | tcctcctgca gtgactttca aggctttagc ttgtcccgtt |
| | accaattctM aagggtctaa |
| 88921 | cctggaaccc ctcaggaagc ctgttctcac tttaYtgccc |
| | gcaacccgca gggcctcatc |
| 88981 | tcccagcccc aaagtgtcag gcttcatggg gaagccctaa |
| | Ycaccccctc ccgacttgta |
| 89041 | caccttggac ttgacRctca cccacagctg ctgtcttcag |
| | cgggggagcc tttggctgat |
| 89101 | tttctgaagg agctcggaag ggccagtgat taaataatga |
| | ccacctactg attcgtgggt |
| 89161 | cacactttca tctcagcacc ttgatttcta cagaagcatt |
| | gactttactt atcgctgtgc |
| 89221 | atggcagtaa tgccaacaga aaacccaccc tataacctca |
| | gctccccagt gaatcaaaac |
| 89281 | tgtccaagtc cctggtccaa taggcccgca tcactgagct |
| | ggcatcatta tctcatggct |
| 89341 | gtaaccagat ctgattgata tttggccagt gagtcccttt |
| | ttaatatagg atttgttaag |
| 89401 | atattgaagt gctctcgaac tggttccagc ctcctagctg |
| | gacaaggagc ccagggccct |
| 89461 | gctccagggg ctgttcctat tggtcactta gcttagctgt |
| | aggaggaccc tgcttgactt |
| 89521 | cttaaaattc tttcctgagt cctctttcct ttctccctcc |
| | cttcctttct gggtcccctt |
| 89581 | ccttcttgtt ttccttcttc ctttctcaaa ctcttccaga |
| | aaggcattaa agtgattgtt |
| 89641 | gaaaatccat acaatgagac aagactccag aaactattaa |
| | gggaagtgag gaaatgtggg |
| 89701 | tgataagaaa tcgggatgat aaaaccctaa cagaggaggg |
| | aggttgcagg cctggccaaa |
| 89761 | ctcactggga acttcactta tatgaccacc ggcggcttcc |
| | atagagcttg gtccttccct |
| 89821 | gatggacaga tggccattta gtgcttggag ctgagtgttt |
| | ctRggggctg ctggctgtYg |
| 89881 | gccctgcttc ctgcatcctg gactgcaaga ctgccaggcc |
| | cagaacatcc accttccaat |
| 89941 | gggggtgcct agaagtccac tctgcaaata gaagctggtg |
| | ttgacccagc ctctgcctgc |
| 90001 | tgcctgcctt tccagtccct gcatgtgggt gtacttggct |
| | gcctgccacc tgtctgtgga |
| 90061 | ctggaKttcc ccaaaggttc tgggtaaccc ctcagcccag |
| | Rtccccagac cccactcctc |
| 90121 | caccccttcc tttgcttttt caggcctgaa gcgcccacat |
| | cagagctgtg ggtcctcacc |
| 90181 | ccgccggaag cagtgcaaga agcagcagct aggtgggtac |
| | cagtgtgggg aggaacataa |
| 90241 | acagagagat ggggttgcct gggggccaag acccggaccc |
| | tggaccttgt cccctgccaa |
| 90301 | taagcctcca gaggcccagt gactcttgtc tcgcaagttt |
| | ccaggagaaa aggactggag |
| 90361 | gtcttgcaca ggcagaggag aggcctgggc tggcagcgaa |
| | ggggctcccc acccatctgg |
| 90421 | ctgcatgggg acttctctga agagggctcc gctgaaggag |
| | ctggaagcac catacaggtc |
| 90481 | tcagcttcgc tgtccaggct tttccaggga caccctatgt |
| | cctcagtgtt gaaggagacc |
| 90541 | cagggaggag accagtttgg gggtgagcag ggatggggag |
| | gggtgaggag gcctgggaga |
| 90601 | aaccttcaag acagaggccc tgggggccca tggggagaca |
| | tggagctgct tagggcaggc |
| 90661 | caggccagtg agagaagaaa ggggacttct ctcattcttg |
| | accagttctc agaatggtcc |
| 90721 | tcgttctcca cgagttgtcc cactaactag agccttctct |
| | gcccccactg ctgccaacca |
| 90781 | tggctataac cagaggattc acatccgctc aaaatagttc |
| | actaccttgg cctttcccca |
| 90841 | tagggttccc ccaaaagcag ccccagccct ttccctgggt |
| | cacctgagac ctctcaatga |
| 90901 | cccaggaatt aaaaggttaa catctggcac tgtaggggcc |
| | cagctcccac agttccgatc |
| 90961 | ggtaatgcct aggcctacag gttgttagtg ctcacccctg |
| | ggcattgcag aactgaaata |
| 91021 | ttgggaagRg gcactgggga tccagatatt acagaaagag |
| | gttgtttcaa taagatattg |
| 91081 | caaagtagct gggcgcggtg gctcacacct gtaatcccag |
| | cactttggag ggctgaggca |
| 91141 | ggtggatcag ttgagcccag gaattcaata ctagcctggc |
| | caacgtgaca aaaccttgtc |
| 91201 | tctactaaaa atacaaaaat tagctgggtg tggtggtgtg |
| | cacctgtagt cccagctact |
| 91261 | caggaggctg aggcaggagg atcacttgag accaggagtt |
| | tgcggctaca gtaagctagg |
| 91321 | atcgtgccac tgcactggag cctgggcaag actgagactc |
| | tgtctctggg aaaaaagaaa |
| 91381 | aaaaagagat gttgcaaagg aggtgcccta ttcccctccc |
| | cccatcttac tgaMccctta |
| 91441 | caccacgtgt actattgcag aatggataga accgtgaccc |
| | acacttcccc attcccacct |
| 91501 | gcctgtttaa atagctaccg gagagcctat gctcccttag |
| | aactgattcc ttctggaagt |
| 91561 | tggggtggca cagatccccc tatggtgctc ttgacacccc |
| | caccctcacc ccatcataca |
| 91621 | tacacatctg gggctacagg ctcccaacag cccaatactg |
| | gggcaagaag acatgagcag |
| 91681 | agggccgggg ggtcctctcc tcatgccatg tccctgcccc |
| | ttgcagagtt ggccaagaag |
| 91741 | tacctgcagc tcctgcggac ctctgcccag cagcgctaca |
| | ggagccaaat ccctgggtca |
| 91801 | gggcagcccc acgccttcca ccaggtctat gtccctccaa |
| | tcctgcgccg ggccacagca |
| 91861 | tccttagaca ctccggaggg ggccattatg ggggacgtca |
| | aggtggaaga tggtgctgac |
| 91921 | gtgagcatct cggacctctt caacaccagg gttaacaagg |
| | gcccgagggt gaccgtgctt |
| 91981 | ttggggaagg ctggcatggg caagaccacg ctggcccacc |
| | ggctctgcca gaagtgggca |
| 92041 | gagggccatc tgaactgttt ccaggccctg ttcctttttg |
| | aattccgcca gctcaacttg |
| 92101 | atcacgaggt tcctgacacc gtccgagctc ctttttgatc |
| | tgtacctgag ccctgaatcg |
| 92161 | gaccacgaca ctgtcttcca gtacctggag aagaacgctg |
| | accaagtcct gctgatcttt |
| 92221 | gatgggctag atgaggccct ccagcctatg ggtcctgatg |
| | gcccaggccc agtcctcacc |
| 92281 | cttttctccc atctctgcaa tgggaccctc ctgcctggct |
| | gccgggtgat ggctacctcc |
| 92341 | cgtccaggga agctgcctgc ctgcctgcct gcagaggcag |
| | ccatggtcca catgttgggc |
| 92401 | tttgatgggc cacgggtgga agaatatgtg aatcacttct |
| | tcagcgccca gccatcgcgg |
| 92461 | gagggggccc tggtggagtt acagacaaat ggacgtctcc |
| | gaagcctgtg tgcggtgccc |
| 92521 | gcactgtgcc aagtcgcctg tctctgcctc caccatctgc |
| | ttcctgacca cgccccaggc |
| 92581 | cagtctgtgg ccctcctgcc caacatgact cagctctata |
| | tgcagatggt gctcgccctc |
| 92641 | agcccccctg ggcacttgcc cacctcgtcc ctactggacc |
| | tgggggaggt ggccctgagg |
| 92701 | ggcctggaga cagggaaggt tatcttctat gcaaaagata |
| | ttgctccacc cttgatagct |
| 92761 | tttggggcca ctcacagcct gctgacttcc ttctgcgtct |
| | gcacaggccc tgggcaccag |
| 92821 | cagacaggct atgctttcac ccacctcagc ctgcaggagt |
| | ttcttgctgc cctgcacctg |
| 92881 | atggccagcc ccaaggtgaa caaagacaca cttacccagt |
| | atgttaccct ccattcccgc |
| 92941 | tgggtacagc ggaccaaagc tagactgggc ctctcagacc |
| | acctccccac cttcctggcg |
| 93001 | ggcctggcat cctgcacctg ccgccccttc cttagccacc |
| | tggcgcaggg caatgaggac |
| 93061 | tgtgtgggtg ccaagcaggc tgctgtagtg caggtgttga |
| | agaagttggc cacccgcaag |
| 93121 | ctcacagggc caaaggttgt agagctgtgt cactgtgtgg |
| | atgagacaca ggagcctgag |
| 93181 | ctgyccagtc tcaccgcaca aagcctcccc tatcaactgc |
| | ccttccacaa tttcccactg |
| 93241 | acctgcaccg acctggccac cctgaccaac atcctagagc |
| | acayggaggc ccccatccac |
| 93301 | ctggattttg atggctgtcc cctggagccc cactgccctg |
| | aggctctggt aggctgtggg |
| 93361 | cagatagaga atctcaggtg agtaagagtg gaggaggacc |
| | ggggagggga ttgggctttt |
| 93421 | gggggagcag aggccaacca gtctagcaaa cctctgccaa |
| | gaggactgga taagaatctt |
| 93481 | gaatatcaga acataatggc ctgcaatgca agagaggaag |
| | ctccatacct tgacaggacc |
| 93541 | tctggagtcc aaatggatcc tcctcttgtc tgtggtttct |
| | ggactctcag tctctctgtc |
| 93601 | aatcatgggc tgaaaccaca gatccaggct gtctaYagag |
| | gctggtggac tttgggcaag |
| 93661 | tatatggagc acacacatgc acactgtcta ggccagagtt |
| | tcttcgttcc taaaacgaat |
| 93721 | ctgtaacaat agccaactca tatggtcatt gcaagaatga |
| | gatgggatgg tgcatgaaaa |
| 93781 | acattttgta gcacagtgcc tagcacagag caaaggctca |
| | gtgtgcatat tagctagaga |
| 93841 | aaaggctagg ctgcagtaac agagaaacct cctccccaaa |
| | tacagtaggt taaataatgt |
| 93901 | agaattttat ttctctctca tggaagagtc agaggtaggt |
| | ggtccaggtc agtggaatgg |
| 93961 | ctcagctcca cgcagtcatg caggtaccca ggtccctccc |
| | attttgctcc attatcccct |
| 94021 | tgggaatcct atctgcacag tcaaagctac gtcatgggta |
| | tatctgtgct ctggcttggg |
| 94081 | aacaaggaaa gagcatggag gaacatgtaa atgcattaaa |
| | gttcagtcct gaaactagga |
| 94141 | tacatccctt ccactcacat tccattggtg agaacatagt |
| | cacctggcca cactgagctg |
| 94201 | caagggaggc tgggaaatgt gatctctggc tgggaaacca |
| | tgttcctcat gatagctcta |
| 94261 | ttgctgtaga tgaagtggag aatgggcttt ggtggacgat |
| | tagaagtctc ggccataata |
| 94321 | tgtatttgtt agtactactg tattaagccc catctctctg |
| | aggggatggc gatgacttct |
| 94381 | cagctctgcc cagcactgat cctctgacac ttcgcttctt |
| | cttatggcag ctttaagagc |
| 94441 | aggaagtgtg gggatgcctt tgcagaagcc ctctccagga |
| | gcttgccgac aatggggagg |
| 94501 | ctgcagatgc tggggtgagc caggccttgg agctgagaag |
| | ggtcttcagc tggggatgcc |
| 94561 | tggtactctc ctccctctcc ttaatcctac acaaggcaga |
| | ttcctggccc cgcccttttc |
| 94621 | ccctcctcgt tcctccccac catctttgct tactctgtag |
| | gttagcagga agtaaaatca |
| 94681 | ctgcccgagg catcagccac ctggtgaaag ctttgcctct |
| | ctgtccacag ctgaaagaag |
| 94741 | tcaggtgagt gatctccagg agggctcact gactggggag |
| | atgagccact gcccaggtcc |
| 94801 | cagagtcccc ctggggcctg catgttcctc caagtctgac |
| | cagtagtttt tcagtgagac |
| 94861 | ccagagaggg aaggcatcag tccaagtact cagtagggct |
| | gagtccaaac ccaaggctcc |
| 94921 | cagagtcccc agagcaaggc ttccttcatc taatatttta |
| | gactgtcttg gaggagcatg |
| 94981 | agtgttaagg ctccctccct ctttctgaaa tcccaggaga |
| | agggatagca gtgcgtaggc |
| 95041 | aggaatcagg cactgtgggt acacataact acatgaagca |
| | ccacacacct tcacatttgt |
| 95101 | tcacacatgt ctacacttgc acagaagtac acaggcatat |
| | gcaagaatat gcacccacac |
| 95161 | atgcccactc ataggtgtgc acatgtatgc agcgcacaca |
| | tgcaccttta actacatgta |
| 95221 | cacattttga tgccagcaca ctctaactaa ctaagcttgt |
| | atgcacaaat atactcctgc |
| 95281 | cctgtgtctg ggactcagta ccatggggag gagtgggacc |
| | agtgaccctc ctccctggca |
| 95341 | gaacgctgct agtttatatg gcacctttat gacatttaga |
| | gaaggcacca cttcatccac |
| 95401 | acagatgcag cctgtcacta aggttcagca cttgttgagt |
| | gaaaataata agttggacca |
| 95461 | cagtccctat tgtcgtctcg gctgggcatc tgtgaaccgt |
| | gattaagcca aggttcagaa |
| 95521 | gtactgaact tgttgacatt tttcttccct ggctattgaa |
| | agtgacttta attttgtctt |
| 95581 | tcatcaccca ccattgttct taatcaccct cactcccctc |
| | aactcatctg tctccccagc |
| 95641 | taggaaccca ggcaaggctg gcttgtgaac aggtggggaa |
| | agtgaggtca gagaggaatg |
| 95701 | cgcttggctc agggctctgg gtgagtcagg gccacagccg |
| | aggctggaag cgatgtctgc |
| 95761 | agcgtcccag tctagacttt actcattgcc tggggggcct |
| | atcccaggca catcccaggc |
| 95821 | ataagccctg ggagcagggt gctaggtctc ctcattgtgc |
| | ctcatgaaga ggccgtcagt |
| 95881 | ctgtcttcta gatcccttga gccgccaggc aaaaacttcc |
| | ccataggaac agaatagacc |
| 95941 | cagaattgag gccagcagag ggtctttcct cctagccgtg |
| | cctgctggaa tccggcgagc |
| 96001 | aggccatcag ctctttctgg gcccctgtct tatgtctccc |
| | acatgagggt ggccatcctg |
| 96061 | tcacttgcta actggggctt gcaagtgccc caaccccagg |
| | gcaaagggac tgggccttgg |
| 96121 | tctcctcgca gttttcggga caaccagctc agtgaccagg |
| | tggtgctgaa cattgtggag |
| 96181 | gttctccctc acctaccacg gctccggaag cttgagtaag |
| | tgatctttcc actgcctctg |
| 96241 | cagcccagtc cctctctata cctgattcac cccactccct |
| | tgcaaggcaa ggaaggtctg |
| 96301 | gggcccctgg ggtaggggat tccactcctg acacctttgc |
| | cacaatcttg cagcctgagc |
| 96361 | agcaacagca tctgcgtgtc aaccctactc tgcttggcaa |
| | gggtggcagt cacgtgtcct |
| 96421 | accgtcagga tgcttcaggc caggtgagca gaaggaaagg |
| | gatcttggcc ttatgggcct |
| 96481 | tgagaaaaga ggaggggaaa gtgggatggg aaatggagga |
| | ggcccctcgt ctgcaggata |
| 96541 | aatctgtgca taccctactg ttttactcac agctttacct |
| | tcagaaccta gctcaggctt |
| 96601 | ggcacctggt acttggtata tattgaatta atggatggat |
| | gaaaagatgg atgtatggat |
| 96661 | ggatggctga aaagatggat gggtggatga aaagatggat |
| | ggatgaatgg atggctgaaa |
| 96721 | agatggatgg gtggatgaaa agatggatgg atggatggat |
| | ggatggatgg ctgaaaagat |
| 96781 | ggatgggtgg atgaaaagat ggatggatgg atggatggat |
| | ggatgaaaag atggatgggt |
| 96841 | ggatgaaaag atggatggat ggatggatgg atggaaagat |
| | tgatgggtgg atgaaaagat |
| 96901 | gaatggatgg atggatagat gaatggatgg atggatagat |
| | ggctagctga aaagatggat |
| 96961 | aggtgtgtga aaagatggat ggatggatgg atggatggat |
| | ggatggatgg atggatggat |
| 97021 | ggatgaagga atgagtcaac aagtctacga agcacaggca |
| | tagccaaagg aataattagc |
| 97081 | tgtgtgttgc cctccatggt gctgaaccaa tatagccagt |
| | tttgatcatg gggtaggtta |
| 97141 | ggtgtcattt ttcttactca taaaaatttc tattaaaaaa |
| | aattcagccg ggctcagtgg |
| 97201 | ctcatgcctg taattccaac actttggaag gccaaggcaa |
| | gaggattact tgagctcagg |
| 97261 | agttcgagac caacctgggc aacatagtct ctacaaaaaa |
| | tgaacaaaat taatggggca |
| 97321 | tggtggtgca tgcctgtggt tccagctatt tgggaggctg |
| | aggtgggaag attgcttgag |
| 97381 | ccgtggaggt tgaggctgca gtgaggtgtg aacacaccac |
| | tgcactccag cctgagtagc |
| 97441 | agagtgagat ctccatctca aatttaaaaa gaaaggaaaa |
| | aaaaaaactc acaacattct |
| 97501 | taatttttat tatatttctt gtgaaaattg tttttctgac |
| | tttttaaaac actcttgcag |
| 97561 | gattcatcaa gcaaagacat aatcagtatt tcacatgcct |
| | gatttaaaac acttttgcta |
| 97621 | gttccgttat gtttccctgt caattgcaaa atcagcttta |
| | tctggatgtt tgaacatttg |
| 97681 | gggcagaaaa gggtgtgcct gtggtgggtg atgtgctggt |
| | ttccagtctc tgggtttcat |
| 97741 | ggcttggtat ctgatcctgc agggaggcgg acctcatctt |
| | ccttctttcc ccgcccacag |
| 97801 | agacaactgc agagctacaa aggtaagaag ccaagaggcg |
| | gtgggcctgg ggccatcctt |
| 97861 | agaagcaact tgggctcagg cagttgaggg gaaaggtgac |
| | caagagacta ggaagagctg |
| 97921 | gggtatggga attcttcctg ctactgctgc actgaggaat |
| | gaattactgc ttaggttggc |
| 97981 | aaaggatttt aacttcctct agYgtgtcca gaccacatct |
| | ggtccctgtg gaaggaagca |
| 98041 | tccccagaag actcagagga aggcaaaccc ttggggagaa |
| | cataaagata ctgttagact |
| 98101 | ggcattacta gatggtgaaa actctggaaa agcccttaca |
| | actgaaaaaa gacagggagg |
| 98161 | gcaattatgg tacctacatc ttagggttgt tctgagaatt |
| | aaatgagtta ctacacttaa |
| 98221 | ggagtttaga gcactgttgg catgcagtgg |

following is a genomic nucleotide sequence of a PROL4 region (SEQ ID NO: 2).

| >12:10841901-10975900 | |
|---|---|
| 1 | tgaggagctt gaccagccct ttaaaccaac tcagcaagtc tgaatgatgt gcaaagggac |
| 61 | aattagatat gggtaagaag tccccaacct caacccataa aggactgtgt ctgaggttct |
| 121 | tacagcatca agggagatgg ataccatggc ttccatcaag gtgctgctag tcctgctctc |
| 181 | aaaggctttg gtgaccctga gctctgctca gagcatagat gatggcaaRa ggaggaaatg |
| 241 | gggacaatac aatggataac ggggttaaaa tgcatggaaa gtactagagt tagattgacc |
| 301 | ctttcagaaa ttaagcactt tattttcaga gagtaggtca ataaaagagg aggaaacctt |
| 361 | aatttctRtt tttcactgat tgcagatact tctctgaaaa tctcggccta agtcttaagt |
| 421 | caacgtcttc atgatgtaat tttgccgttc taatgtatat ggattttcag ttgttgattc |
| 481 | tgtttgtgtg aatcagggca tctaaattga acggaaaatc tatatttttc atttctataa |
| 541 | tctataatag aattctataa ttctataatt catctagata tcRcatttcc ttgacatcaa |
| 601 | actcttgaac atttaaaaat attgataatt ttgattaaat ttgtacttaa tcaaaatttt |
| 661 | gatgctaagc aggcactatt aaattctttc tattccattc cttaatcctt taaaatacag |
| 721 | gtcacccctc attcagagtc aatgattggg actagggttc tactttggga aactttggtt |
| 781 | tattgtatcc aaatctttat aatttttttt ccctaaagct ctcttaggca tttttaaatc |
| 841 | tcttgtgtct aaacctaagt gtctttctag aaattcacag ctggggtttt atcaaatcat |
| 901 | gttttaaatt tgtcttcttt ttcatttttt tttttctctt aaaatcctaa gtactgttgc |
| 961 | tgaagagttc tttcaagagt aaggtaaacc tttattcagg gatgtgttct tttctttgtt |
| 1021 | cttcagaagc taccatagtt ttcttttttg tttaataaac attctcaaaa ttctcaaaca |
| 1081 | ttctcaaaat tgagtttagg gagtcccaaa gggaaaaaga gaattacaaa atctgaaagt |
| 1141 | tttagtggtt ctcaataata acattgattg attgagacaa gcctcctctg tcacccagat |
| 1201 | tggagtgcag tggcatgaac acagctcact gcagcctcca cctcctgggc tcaagctatc |
| 1261 | ctcctacctc accctcctaa gtagctggga ctataggcac acaccatcac acccagctaa |
| 1321 | ttttaaaatt ttttatagag atggagtctc accatgttgc ccaggcttgt cttgaactcc |
| 1381 | tgggctgaag atatcctccc acctcgtcct cctgaagtac tgggactaca ggcatgagcc |
| 1441 | accatacttg gcccaataat gttaaatatg gaccatctcc ttcattttca cattagaatc |
| 1501 | tcttgtgtca gagttgtaat tagcatttga acatcagcag aatatagaca cagtacttat |
| 1561 | ttgtaaatgc ttttctctaa atctttctac aaccaatggc taatgattga ataacacaaa |
| 1621 | tctcccattt ttgcaagagc aataactccc ttgacatata actaatttga agcataaaga |
| 1681 | tacataaaat ttgcctacat tattgtagtc aatcagtgaa tgtatcattg cactttcttt |
| 1741 | tgccactctg aattaaaaca caaatgcttc attctttagt tttcaagtcc tgataactgc |
| 1801 | ttaaacacta atttgtcaca gagtaagtga aatttaaagc caccattagt cacacacagt |
| 1861 | ttacatgaaa aatttgaagt ccattctaat ggcctgggga gagactctgt gtaggaggaa |
| 1921 | ggatataata catgcccaga gtacatgaga gagctaatga attatgccac caaagaagtg |
| 1981 | gcagaattga gtaagtgagt caaccaacaa ttttgttgtt caaagaaaac ttttatgctt |
| 2041 | aatgattttt ttcccctgtt tctgacttgt caatgaccct tctactgagt ttatgatgtc |
| 2101 | atttcaaact tcattctttt tctcttcttt ctttgaagga ggctttcaag aggaagatga |
| 2161 | aaagacctaa ctaccatgtg agctattttg agttcaatta agttattaag aattgttcta |
| 2221 | aatggattca acgaacctct gggttactgt tctaataaaa catttctaca agtttttact |
| 2281 | gtaatacaaa taatcatgtt taaccatgaa aacttttttt ctctctttat ttacaaatat |
| 2341 | tttctttttt gtacacaaag ttcctggcta ctctaggctg gttagtacag ggttgatcac |
| 2401 | cctaaaatat ctttgccaca ataYgaatga tgtgcttttg atggtatgtc ccagatacat |
| 2461 | atttcctttt gtggcctgac atatggcatt gtctggagaa tattcaatat atgcttgaaa |
| 2521 | agagaatgtg ttgtctcttc cttttatgga atctttcata aatatcagat caggttgctt |
| 2581 | gatagtgttt gttaatgtat tctatattct tacagatgtt ctgttgttca acaaattact |
| 2641 | gaaatagaat cattgaaccc tttaaaaaaa gatgtcttat atacaatcca aacaatattt |
| 2701 | ctctgggatg tagagatgat ctacattgag gcaatatatc ctttgagagt tttgacattt |
| 2761 | tagcataacc atgtattagc ttttgattga atgtcttgtc acagcatctt gataatcatt |
| 2821 | gcctggtgca gtccgtggac atacgtcttt tgtggagaaa ttttcttcaa taaataatta |
| 2881 | atagttcaat ttaggcttct tattttatag acattttcta cataattttt ctcactttta |
| 2941 | cttatagatt gtccttcatt cattgatctg caattctcaa ataatctttg aacttactgt |
| 3001 | gacaatatat ttagccataa tatatacctt ttctgtgatt ataagattat tcatagaatt |
| 3061 | ccacagcatt tattaagaac tgtcacttta ttgagtcttt cccaaaagta gagctttgtt |
| 3121 | ggatgatcca agaggaacca tctaacgcgg actatgagaa ggaactagtt gcttttcata |
| 3181 | aagatgcaaa taacagcaag atgctgttac agttgttttg aattagtcac acaactttgc |
| 3241 | atctggaaac acggtttaga actatgaaaa tgccctctac tcttgatagt tgatattagg |
| 3301 | atattaattt ctatttgctc tgaaacatgt tttaaaaaac agcaactcta aagaaaggaa |
| 3361 | gagttacaga agagagaaac agggatacac agacagagtc agaaacagag acagagataa |
| 3421 | agagatccag ccagctgtac ataagattcc ctacattaca attacagata cactaattaa |
| 3481 | gttttaggag taattaatca cataaaacat taatgcaaga actgaagtta atttagaaac |
| 3541 | atcttatctt tgtttgtcct agaaaagcat agtttctctt cagatttgtt tatgttgttg |
| 3601 | gaattttctt ccttttagaa tagaYattac tttccaaatc acctttagag atgcatgtct |
| 3661 | taatttattg ttccccagta ttaggataaa tgaatgactt gaggggtaKa ttagagctat |
| 3721 | ggactcacca aaaatcacag ctaattccgW ctctggcata aagtagctgg aggtggcaat |
| 3781 | gagaaaggac aaatagtagg caataaagag gagaaggaag gaaatgacag ctttcagggc |
| 3841 | tctcacatgg gcttctgtgc tggggtctct gcaccctgtg gcactgagct gcattcgcct |
| 3901 | gatatgtctc cgcagggaga ggatcaagag gaaaaaggac attaggcaca cacaaaaggg |
| 3961 | gagcagcgtt gccaggttga gaaataactt ggtagaagca tgttgagttt tatttactct |
| 4021 | gcaactccaa gttaagtttg ttttcctctt tgccttcaca caaaacctga aatcagcgtt |
| 4081 | caaattctca gtggctggaa ggctaataaa cacagagaga accacgcacc ccagtagaat |
| 4141 | ccaggaaatc accctgtcaa ttctccactt catccagagg aaaagtgggt gaaagaaatt |
| 4201 | acctatcttg aagaaatagt aaatgctgag gcaggttgca aaccagatac ttaaatgatt |
| 4261 | ggttagtgtc cagaagaagt caatgattct catttcttta ccagtggcat agacatctgg |
| 4321 | atatagcacc aatataaaac aatctaatag tattacgcac aatagacaaa ttctggatat |
| 4381 | ggccagactt gtgaggatta aatcaatgga ggcaattttc ctcttcttga cccagtccat |
| 4441 | gcagtttacc aatccaatga atgcattccc taagatcccc actgaaaact ctccaactgc |
| 4501 | taagaacaat aaagtagtct gcactttatc tgccatgttt aaatatgcaa ttagtttcta |
| 4561 | gttgacctga tggagtttga catccacacc tgcttcttag attttgatgt agttttcttt |
| 4621 | acctctttgt tgtagtctgt tttgtgatgg agactggaat gataaatgaa gactggagct |
| 4681 | atcttcatta aatcctaatt agtcttgaca aacaatgtgt ttgScgatcc ttggcctggc |
| 4741 | cactgtatgc ccatttacct actcagcagg ttgtaatttt Ycacatacgg ttaatttagg |
| 4801 | ctgagctact gtttactaag atgcaaattg ggccaattcc ctttcatggt cctgcattgt |
| 4861 | ctttctgagg ctaaggtgta cgcattttgg attcattaat ttgtgatgta tgctagtaac |
| 4921 | ctcaataggg ttgctagatt tagcgactaa aaattcagga cactcagtta aacttgaatt |
| 4981 | acaggtaaaa aaatcaataa ttttttagtg tatgtccctg attacttaag atcagtgctt |
| 5041 | tcaggaagta ctgaagttca aattagattg agtgttccat tctggcaacc tgaacatcaa |
| 5101 | gaatttggta gcaagtataa aactggtaaa attggaaagt tcttgaagaa tttttatttt |
| 5161 | gctgaatgtt aatattgata attagtaaag aatccaaagt ttggttccat agtgagagtt |
| 5221 | atatattttc aggaacttta aaaagttaac actaaatgta ggttatttga ttccaaagaa |
| 5281 | actttaggtc attattttgg ctgttcagat gaacaattac atggcatagt gtctcttaat |
| 5341 | acaacacatt gtcttactga ctaaatgact tttaatggtt atttagcaat gttatatata |
| 5401 | ataaggtaga ggacctgtaa taaaaaaaga ctattgatta ccttaaattt ttcattttca |
| 5461 | cttaaaatgt tctcaaggaa actttgagag gtattagata tgtccattac cttgtttgtg |
| 5521 | gtgaagatat catgggtgtt tgaaatttta tacattaaat atatgcggtt ctttttatac |
| 5581 | caattatact ttcataaaga tttcaaaaat ttaaaaaatg ttctcaaggg tagttgtatg |
| 5641 | catgagagag gagatagtaa aattctgtat ttcattctct ccactgtcct ccatacaaat |
| 5701 | tgcatgagta tctagatcct caacccttct agcaggagac tgaagattaa actttccaca |
| 5761 | tctctagaga tgaattttta gtttgaaata gcttctctaa tttagagtta ctgagaagcg |
| 5821 | ctttactaca tggggttcca tagaatagtt cttgggtatt cacaccttcc tgggaatgca |
| 5881 | tgtcttctct tatccttcct taaaggacaa accatcattc cggagatgta gccttaactt |
| 5941 | ttggacaatc tgtatgatta tgtagccatg ttcacaggca actggaacat cacctatgat |
| 6001 | aggacgatct tggatatgat ccctgaccta gacttatcat ttcttttgtt ttttttttga |
| 6061 | gatggaattt cgttcttgtt gctcaggctg gagtgctatt ttggctcacc gcaacctcca |
| 6121 | tctcccaggt tcaagtgatt ctcctgcctg agcctgccaa Rtagctggga ttacaggcag |
| 6181 | gtgcaaccat gcctggctaa ttttgtattt ttagtagatt ccgggtttct ccatgttggt |
| 6241 | caggctggtc ttgaactccc gacctcaggc gatccgtgtg ccttggcctc ccaaagttct |
| 6301 | gggattacag gtgtgagcca ctgtgcctgg cctagactca tcatttttac cctacagttt |
| 6361 | atcctggtaa atgaagtgct catgggaact atgatgatgt attttctcaa ataattttta |
| 6421 | gacttgtttt tgtttagttt tggaaataaa tgtagatttt atgaatacca atgttgtata |
| 6481 | gcgtgaagtt atgtgaatat atagttaaaa aaaaagttct atgagagagc attttgtcaa |
| 6541 | aagcttacta ctttgaaccc cagtcactgt ggtcactaga gatgcttgaa acattagaga |
| 6601 | ggcaggaaac aaaatatgga agaaaaaaac ttaaccctaa tggggtaaaa ccttcccaaa |
| 6661 | agtactaatt caataaaacg tttatttcac ttaaacctta ggaaaaatat aaagttcatc |
| 6721 | ctagtataaa atctacctgt attttgtaga atacattaat atatttcatc aagagaacat |
| 6781 | gaattcttgt gtcttgtaaa acaaagagta acatatacag ctagcatatt agaaatatag |
| 6841 | ttcataaaat ttatatactt ggggctataa atattagtac tttacagagt gagttttaca |
| 6901 | taataataag acaaacaact gataattttc cggtaattag tattttagtt ataataaaag |
| 6961 | gaaattagta gattgaaata catcagaatt aaacagaaag taatgggagt aaaagcaata |
| 7021 | atgtacagcc atattagtac atacacaacc tgagatatgc cagaccaaac gtttcaataa |
| 7081 | tattatagtg aggatgKatt tcatgttcac agaaaaatta aaaattttag tgacttattt |
| 7141 | tccttttttt tttaataaaa atttatctgt attttttgta aacattcgtt tcttcttcca |
| 7201 | gggacagtat gtgcttattc catggtatac gctattttct tcccaattta tttcttttgt |
| 7261 | taagtcctga gtgaaaacct ttgcagggtc tggttagaga aaagaacctg attgagcagt |
| 7321 | ctgactaata tttgttatcc ccattctttt atccagattg tgaagagggc ttaggaacac |
| 7381 | cccttggtgg gtggcacttg aatgggttcg attcccacct aggtgctgca tgaaaagcaa |
| 7441 | gttgtttgag aggaaattta attccaagga aaatcaatag aaatcatcag aataaggtca |
| 7501 | accagaaaat attacctagg agaccatgga aatcatcatt cgggaaaacc aaagacagac |
| 7561 | cagagaacat ctgagttcct aatcaataag ccaaatgggg gtgactcagc agagccatga |
| 7621 | aaggagcaca attaaggagc ggcaataaaa atgtatgaca aagaaaaatg agaaaccagt |
| 7681 | tactgtagct caggtccact ttaccatgag ccaaagtaaa atctaaaaag actttaacta |
| 7741 | ggcaatacag gcctaacact tgcgttgagg agacaagaag agaagcatga gctctgaaat |
| 7801 | tcttcgagga aagggagtgt aaagatgagc atttctgagt tgtaaaggga tagaaaagat |
| 7861 | gtattcagta gtttgctact aggtcctaat aatatttaaa gatatgcatc caaaataaat |
| 7921 | gagataactg gggaaaaatg agaatagact attaagaaaa ctatattttt tgttagagaa |
| 7981 | gatttggttt taaattccat ttatacagct aagatttcat atcaYgcagg caatttttct |
| 8041 | acatgtcagc attctgacaa atgtctgcct cagtttatta tttaaaacaa ttaaaataag |
| 8101 | tgagtgaccc aaggggtaga gaattgctgc aatctctcca aactccacag ctaacttgta |
| 8161 | ttttgtcata agatagctaa aggtcatcaa aatagaagaa atatagtata ggaaaaaaaa |
| 8221 | gaagataaat gaagtcatag ttttaatggc tctcacatga acttctgtgc tggggtctct |
| 8281 | actgccggta gcatagagtt ttatttgctt ggtatgtctc cataaagatc ttactaaaag |
| 8341 | gaaaaatgat atcagtgaca caataaatgg gacaattgca aacaggttaa agagagtYaa |
| 8401 | gggttcaaag tatggtattt tactcacatg gaacatttca gtaatgtttc ttttatgttt |
| 8461 | ggcaattgca tgaaacctat aatcacaact cagtactatt gctgctataa ggctgaccaa |
| 8521 | caaggaaatg gcaaagcatc ccagcaggat ccagtgcacc accatatcaa ttttccactt |
| 8581 | cagccagaga aaaagtggat gagaggaact ggctatcttc agaaaataga agacattaag |
| 8641 | gcaggtggta atccacatat ttaagtagtt ggcaaatgtc cagaaggtaa aaatgactat |
| 8701 | ctgttgttta ttttttgtat aaacatctgg gttcagtact attacaatgc catttacaac |
| 8761 | cattacactg atcaaacaaa ttctggcgat aactaaattg gtaaggatgt agtcaactgt |
| 8821 | ggaaatcttt ttcttcttaa tccagtcaat ccagttgact agtgcaatgt atccattccc |
| 8881 | caatattcct agtatgaatt ctccagttat taggattata aagatgttat ctgcaggact |
| 8941 | gaacatgttt gtagagagaa caatctgatt tcaaatatca ctgtagatga gctaatttac |
| 9001 | aggtgacctg ttaaagcatg atagatcaat tcttcgaatc atgcaataat gttttcatct |
| 9061 | gctgttattt cgacgttgtt attcttccgg aagtgttcag ctctcctctg aaataggatt |
| 9121 | gtctctacac tcctgaagat gaaaccaact aatgagcagc ttgactagtt atttatagcc |
| 9181 | tggaaaaatt ataattatta tcctttagtg agtgatacct gaaatgtttg gctgaaattg |
| 9241 | ctcccatgca aattcaaaga gattttatat catgaatttg caacaggctt cttgctgtag |
| 9301 | ctctatattt ggatcttcaa attgagtttt gaatagggag tcataaaaaa agagataaat |
| 9361 | gtttaataaa aacccaactg tgtaaataaa acttcatttt catcatgtaa gttataaata |
| 9421 | agccacaaat cagttctccc agatgcaaag aaattttcac tttggaagta attagtacaa |
| 9481 | ttagattgta gatatttaac atttagtcag aggataataa ttcttgtctt catttaaacg |
| 9541 | gctaattagg gaaggatata atacaaatct aattagaaat gacttttttt taggcaggca |
| 9601 | catgataata tttactaaga atttgctttc ttttcaattg ttcctcagaa gtgaaatatg |
| 9661 | ccaggtaaag atttgttatc tttagttttt ttttaatgtg acaaataccc ttgaagacaa |
| 9721 | ttcatttcct gaatcactgt ctcttgtata ataagcattg taatctaaaa ttaagaaaat |
| 9781 | ttaaatttta cattattcta gttccaatca actcctttta taagatttgt aatctcacag |
| 9841 | tgcatattat agtcatattt ctagataggg caaacacata aaaaagtaac tacttgggag |
| 9901 | actgaggcag ggggatcatt tgaaccggga ggcagaggtt gcagtgagcc aagatcgtgc |
| 9961 | cactgcactc caacctgggc gatacagtga gactctgtct caaaataata ataataattt |
| 10021 | ttttttttta cataccagta tttccaagga ataggactaa ttctactatt aaattagatt |
| 10081 | tatttgtttt ggggtaaggg aaagtacaac ttagcccaaa atacatttga atgtaggtaa |
| 10141 | tacRaatcac aataccctag ggcttcttgt atttaaatag ttctacaatg tatacaacac |
| 10201 | atgcaggatt ttttctattt cttacatgtg tgtaatgtgt aacaggaaag cataaactta |
| 10261 | ataacttaaa gaaacctagt tatctataaa ctactgctgg aaacaaaata gaaaagaaaa |
| 10321 | tgatgcagag ggaaaaaaat atgtcagaga tttctgagtt agaaggctag agagaacatt |
| 10381 | ccaRggaatc tttgggaaat gacttctaaa attgccatca ccagctagag tactcatgaa |
| 10441 | cacgctgact actgggaccc tcaaccttct ttctcaaggt ggttctgcta tacataactg |
| 10501 | tgcctcctct ctctgtcaaa ctttcattct tgagattata ctccttttcc cacatcatat |
| 10561 | atcatcttag atagaatctc attaaagatt gtttttctcc cttcttgatc aagacaaacc |
| 10621 | cttccagaaa tggagaggaa agggcggtga agataaggac cggccttcat ttcaaagtag |
| 10681 | agagttgttt tgcatgattg attttatagc aagggttgat gagttctaaa atcttttccc |
| 10741 | caaagcctgt ttgcctgaca ttcttctact cattgagttc aaggcctttg ccagtatttg |
| 10801 | tcagaaaaga agtagaacct ctttgtattt agtcaatgac ataaaatgat gaaatgttag |
| 10861 | tgtgggattt agatgcattt tttttttctg caacgacaaa gaatatacgg tgcaatgata |
| 10921 | acattagatt atccattaaa caagacaaaa gtgtgaagga acacaaaaca aatcttcaga |
| 10981 | atcatgacag atattaatga cttccaatag catataagag aaaatgaatg caaacaagag |
| 11041 | aagggagaat gccttttgtt gatttctttc atccttgtag tcaggatact ctttaggggt |
| 11101 | ctctatggaa caaaaRgctt tcttcttcta aRgaaacact tcacaaatct taacatcttc |
| 11161 | agaaaagctt ccctcaactt gctatttccc ataattagaa tgaatgaatg gcttgatggg |
| 11221 | aaaatgacag ttactatgtc accaatcatc aacactaatt ttccctgagg aatcagagcg |
| 11281 | ctagaggtca taacaagaaa gactgggtag tacacgatga ggaggagcag aaagatgatc |
| 11341 | actgccttta tggccctcat gtgggcctct gtactggggt ctctgaaccc tgtagcatgc |
| 11401 | agtcgaatct gcttggtgtg tctaactagg gagaaaagta acaagaaaaa tgagatcagg |
| 11461 | caaaggataa agggaaccat cRcccccagg ttcagggtta actgtttgaa agtacctgga |
| 11521 | attttactca ctttgaattt ccaagtaatg ttttcttcat gactgacttt gaaaaggtga |
| 11581 | taccacatat catcattctt tggaacacta ataattaaag agataagaaa ggaccccaga |
| 11641 | agaatcgcaa gcatgacctt gttgatcttt agcttcagcc agaagaaaaa tgggtgcgat |
| 11701 | atattggcta tcttgagtaa atagaagata ctgaggcaag aagtaaacca gagacttgaa |
| 11761 | ttattggcaa atgtccagac aacattcaca atgcttacta gcacgctatt gccatatgta |
| 11821 | cctggaaaga gcagcataaa gaagccatct aatgatatta cacacagcag acagattctg |
| 11881 | gagatggcca agctgatcag gatgatgtca atcaaggaaa tatctcttct tttgagccag |
| 11941 | tcaatgcagt taactagtac aatgaatcca tttccccaaa tccctatggt caattcacca |
| 12001 | gcaattaaaa taatatatat tgcctctatt gcacttggca tgccagcaaa gacttgtgat |
| 12061 | ttttgaatat cactgatgat gaattgactt tcagctgacc agtgaagaac aatgtatctg |
| 12121 | tctgcctctt agaccatgat atagttgtct ttatctctcc tttttgttag ctgtgatgca |
| 12181 | gatgaagaag atagccagtc ctgctctggg ctagagctgc ctaaagactt caatgattgg |
| 12241 | ggccagtaat ttcctttggt gcagtgatga ctgaaagcct ctaggatgca aatgggaatg |
| 12301 | gatctgattt cttaaatttg cacactcttt cttattttaa gctctgtata ttttggattt |
| 12361 | atcaatctga gctgttgacc aggaacttca agaaggtatc aattacttgg aaaatatggc |
| 12421 | tagaatttaa tttgctggat gactatccat ccttgttagt atattgacat agttattgct |
| 12481 | attacagatt ctaggtttac tgtgttagct tgtgtggcca gtttgcttgc ttggttgact |
| 12541 | aaagctttaa acaaattatt attaacatta aatgaggctt aaatggcata actggtggaa |
| 12601 | tgtagaggaa agaatctaaa ggtgtaggaa gataggagta ttaaggtgaa tttatcatat |
| 12661 | gcgaacttca ccttctcccc attatatgac ttaagagggc caaaaggaca ctccattcac |
| 12721 | tagggcattg agaaatacat taattaggta attcccaata tcctcaaaaa gctctgcagt |
| 12781 | gtttgtcttc tcaactgaag atacaatgag ggaaataaac atgacattgt agtgttgtca |
| 12841 | gcaaatgaga cacggaaaat tctggtttag atgttagggt ttcttagagt gttcttgctt |
| 12901 | tgtacctgaa tgtagcttcc atacatagaa aaaatagtca aaatactggt cacttatctt |
| 12961 | tagaatggaa acagatcagc ctgaacagtg aggaggacaa agagcaagaa agtgatattt |
| 13021 | ttggaactaa aaagcgtgat agatatacaa gactcttcag tctgaactga gtgataagaa |
| 13081 | tacatgctct gaattttctt agctctattt tttggcagca ggtgtattat aaatattagt |
| 13141 | atgctaatta tacacacatt ggaaacacac tgtttcattg ggacatattg aaacatgtca |
| 13201 | ttgggcatag tagaacatat ttgaaataat agaaaaaggc acacagcaat acacagttcc |
| 13261 | cagaaatatt attttgaaac taatattaaa ttttaaggca atattaaata ttgcttatca |
| 13321 | aacaaacaca acaatacaaa gtatttaatg agataatgtg tcatatgttc agggttttag |
| 13381 | acaaaataaa gagataaaaa ttaatcttgc agatttttat gagaaagtaa atagctattg |
| 13441 | ttattctatt cagtgaaatt tggggtttct ttcatgcttc tctgtgaatt ttgtttctga |
| 13501 | agaaatatca gattatacaa ctcaaaaata cacactgtgc ttgtcatgac caaagccata |
| 13561 | aaacaggtaa tattctgaga aggacttcac tagagtaaag aaattgattt gcatgcaact |
| 13621 | gacagacaag gtcgcaccat gttcacatct cagggaatgc aatatttcat tattttaatt |
| 13681 | aaactttttg aaagataaaa ttttgtggag gggaataaag taggttcaag gttattattt |
| 13741 | ggaaatggaa ctactctggg atcaatttgt aatgtccaga aaagatgaag atgaaaatat |
| 13801 | atcatgtagg agggtgatta ataactgaca tattaaaaat tcctggtaaa tccagatata |
| 13861 | gaatccaagc ttgccttctg aatcatggtc tttcccacta cctcacttct tttccttagc |
| 13921 | cgtgtggaat actgatcaat ttaagaaagt tatgcctata ttcaaatgta aacgaaaata |
| 13981 | tgctatagga tatgaccaga gttacctaag ggcctatact tactaaaatg ccaccctctt |
| 14041 | ttatctttcc ttgctataaa taaaacccaa atgatctcaa tttatttgac accgttgaat |
| 14101 | atgttttttt tgtgtgtatt gtgtgtgtgt gtgagtttat gtgttatttg ccctttggat |
| 14161 | tttatcttag cattgagaag ttaaagaggg atcctagctg acagacttta acatctattt |
| 14221 | tcattgcaga aacactttaa tctagaatgt tagtttattg attatttctg tctctactag |
| 14281 | aaaacagatg aaatactggg aaacttatga catcatttcc tgtatgtctt tctatgagaa |
| 14341 | gtgagcaaac acaaacctac ttaggcagat atttcttaaa tttcattttc tttcctttgt |
| 14401 | ttaaatactt tttaccattc tttagaacct tacatctttt acaggcattt gtccttgagt |
| 14461 | tatgccactg attattactg atcaagagaa atgactatac tcttcagatt tataaatcaa |
| 14521 | gaacaggata ctgcgatact gtcYtatttt ctgacaactt atattttgaa tagttgctga |
| 14581 | ctttatctcc taccccattc taaactcttc actatatcca ttgggatccc tagtaaacca |
| 14641 | ttagcagaag ccatatattc tcatttgttt ctctaacagt tcactttatt ttcttcattt |
| 14701 | gaactcgagg ttctggaact tttttgttca atggactact ttgccagtct ggtgcatgct |
| 14761 | ataatttcag aataatgttt ttaagtgcat gaaataaaaa tatttgaaat gtataaggaa |
| 14821 | tccatatata tatgcctttt tcttgcagta tgaaatatat tgttaatata atataggaat |
| 14881 | ttatgtgctt tttaattgac ataacaaaaa tatctggcag ataaagacta gtggtgaaag |
| 14941 | taaatgacaa taggaggtac ctgtaacgac aaaaattttt ggcttcctga agtgaaaaaa |
| 15001 | ttacagatac tgctggtatg gtatgtgctt gttacttaca ttcaagacag aagaaatact |
| 15061 | agacttaagc taaggattaa taaaaataag tatataattt ggtgcccctc ccagttcatt |
| 15121 | catcactcat gtccatgaat cttttcattt taatactcta caattaaatc ctgtggcagc |
| 15181 | tgttttctct ctcacaccct catagaagtg ggtctaaagc tgggatgggt gtttttcttt |
| 15241 | ccctctcgta actttcagat tactctactc tctttccttt ttcctttttt tttttttttt |
| 15301 | ttttgagaca aagtttcact gtgttgctta ggctgttctt gaactcctgg cctctagtga |
| 15361 | tcctcctgcc ttggcctccc aaaatgttaa gattacaagt gtgagtcact gtgcctggcc |
| 15421 | agattattct ccgtctaatt cctcacaaga catacagcag ttcattatct cctgtcatca |
| 15481 | ctctatacaa aatattacct ttccttgtca caatcataag ccagtcttcc ttccttgatt |
| 15541 | tttgctttct acctgaaatt aagtagttaa atatactaaa tttcaccacc attctttttg |
| 15601 | ctaaagttcc cctatctttt tgttggatga aataatcttc tattttttaa aaaaattcat |
| 15661 | tcagtaatct tgtttgttgt atttttcWtt ttttaaaatt taattatgga catataatag |
| 15721 | ttgtacagat gtatgggggt acatgtgaac ctcatgaaaa cagagcaata taaagacccc |
| 15781 | tgttgagaag ggataacatg aatgagaaat atgcatgtct ctttaagaag aggaacagtc |
| 15841 | ttaattaatg gataactata tagagtcaat aattcaagca taaaacactt tattataata |
| 15901 | gcatattggg aatagtcttg tctatttgta taaaaatggt gaagaaatag aatatttggg |
| 15961 | atccatggag acaaataatt tatttgctaa ataacaaaat ttggacaagg cttgaaatat |
| 16021 | atataacagt taagagcttc agctctggtg ccagacagct ggagttgaag cttattctca |
| 16081 | gttttaatga ttgtgtgatt taaattgtgc tgaatctttt ctttctccaa tgtgtaaaac |
| 16141 | attgatcata gtgctatcta tctcatagaa atattatact taaataaagt cttgcattta |
| 16201 | aaatacttag aatagttctt accatatata ctcaaaaaat tttaggtact attattattg |
| 16261 | aattttttgg cattccattc aataaatcac gattctctta tatagttgtt ttgctataga |
| 16321 | gcctttatta atgtgtctta attacaattt cctatccata atatttggcc aaatttcaca |
| 16381 | ttttatgatt gccatagttt gaatgtttgt attttcttca aaattcatgt ggaaatgtaa |
| 16441 | tccctaatgc aacaatatta agaggtgggg ccttttagga gatgattatg atgggtttgc |
| 16501 | cattatgatg ggattaaaag gcctgaaggg aactaaatag gtcttttaac ctttcccctc |
| 16561 | ttctgccatg aaaagataca gcattcaagg ttccatgttg gaagcagtga cgaagccctc |
| 16621 | accagacacc aaacctgcca ctgccttagt cttggacttt ctagccttca gcactatgaa |
| 16681 | aaaataaatt tctgttattt ataaactacc tcatctcaga tactttttat agcagcaaaa |
| 16741 | tggactaaga caatgccagt cacatgaaat cataaaatat ggcaacataa tagttatggt |
| 16801 | ttaacaattt gttttgtttt ccattactca ctgaaaatgt aaacaaactt aaatggactt |
| 16861 | agttgttttt ttccttcata tgtagttctt ttctttattc aatattaatg cccagtgcat |
| 16921 | ctcataatat taatggacag gtgactattt tgtttctctc tgcaatggca taaataaaat |
| 16981 | gattttaaca gcatttcaac ttaaacacaa taaattattt atttatattt gacaacacat |
| 17041 | ctaaatgttc aggttaagta aaaatattct cagatatata ggtttcagac gtttactaaa |
| 17101 | catagaactt ttttcaacat tttggaggct acattaacaa aaactataat ggggtgagga |
| 17161 | gatacctata tgaatatgtg gagtaaagtt tataagaaat tttgtaaggc ttaggatgtc |
| 17221 | aaaaaatttt ttaaaaataa gaaaagaaca tttgtatact ataaaaatct taaaactcca |
| 17281 | gttgtaggta gaagatcaac acataaaact Stgttgtgtt tctacacacc agcaatctga |
| 17341 | acagtgaata taaaaggtaa attaagaaaa caattccaaa ataatgaaat aactaggaat |
| 17401 | aaatttcacc aaagaagcaa aatacttata tacaaaaatt gatgaaacat tgctgaaaga |
| 17461 | cattaaggaa gacacaagta agtagaaaga catcccatgt tcatggataa gatgacttga |
| 17521 | tactgttaag atatcaatac tacccaaagc aatctaaaga ttcattaaaa ttcctatcaa |
| 17581 | aaatttctac agacttcgtt tcagaaagag gaaaactaat gctcaaattc aggtgaaatt |
| 17641 | acaagggaat ccgaataccc aaaacaatct ttaaaaagga gaacaaagtt ggaaaactca |
| 17701 | catttaatat ttttatttta tatatatata tatacatata tatatataca tatatatata |
| 17761 | tatacacata tatatatata tatatatata tatatatttt tttttttttt tccctgtatg |
| 17821 | cctcattaag Kcttccctga gccctgggct ggcttcttcc atggaagata gcagaagaag |
| 17881 | caaggcttgt ccctgttcac agaagatgct agccagatca ggagctgaag atagcaaaag |
| 17941 | agggagaagt ccaacccaca gtcccaggcc ctggtgagta aggtctggtg cgaggagaag |
| 18001 | gctgatgata tggaagggtg ctccagtcct gcttcttctg cttggaaatg gagaatccgg |
| 18061 | agaacctggg ggatgcaaga tggacaggct gctccttgag aacatccagt ctcctagaaa |
| 18121 | aaatgggtgc tctcaaggtg actgaatgaa ctcaactatg ccttgttatt ttaaaaccca |
| 18181 | ctgtctgctt tctgaattga caatcaagaa tccatgtgtg tcttaaacaa caattgttca |
| 18241 | ttgtagtaga acccactggg agaagccttt tggctcagga actccatcta gccacagctg |
| 18301 | attgaattag gggtggatac ctaacccaag tggagccaat cttattctgt gcccactgca |
| 18361 | atgtaagatg taaaataggt atttttgtct ccttggatca gtgctgtatc tttagtgtct |
| 18421 | agaattctgc ctaccacaca gtaggggcta catgaatatt taatgaacaa atgaatgaat |
| 18481 | aaataagtct ctgtgaggaa tttggaattt tgaccttgga aacacagaga atggcagttg |
| 18541 | tggaaactga gtcacattgg ctgcataagc tagaaagaac acccactcct tcccatgtct |
| 18601 | gaggtcttcg ggtctatctc agtcatgact gagtcctgac tctttctaaa gatgattgga |
| 18661 | ctcctcttaa ctttaggaca gatggctcaa catcctaact ataaaactcc ttcctccttt |
| 18721 | cctgcttaca ttcatttctg ttgcaaccaa aaacctttaa tgacatgctg agaaattttg |
| 18781 | caccttgaca tattctgttc cccaccttag gagcttctct aggagtcata atcactcctg |
| 18841 | gctgtacctt gggctgcatc cagatctgcc ccaatgccac cacccccagc tctgtgggct |
| 18901 | ctggctctaa gactacaggg acccagaagc aggaaagagt ggcatcatgg aattccatgt |
| 18961 | cctttagggc aggtgggagg tactatgcct cttctccatc caagaccgct cactacatcc |
| 19021 | tgtcctcaga tatggctctt gggaccctca agtcagatac tcctaatttt tcatctgctt |
| 19081 | ttgctctaca gacagaaaga ggaggtgcca aagaaatcta cagattcatt caaatcctta |
| 19141 | ttaaaaattc tcacagactt tttttttcag aaacagaaaa attgatcctc aaatccataa |
| 19201 | gaaattacaa gaaaccttga atttccaaaa caatcttaaa aatggagaac aaagttggaa |
| 19261 | ggctcacact taatatttta aaaattcact acattacttc tcagcctttt ggctaagatc |
| 19321 | aagtataaaa cttactacaa agctatggta atcaagactg tgttttattg gcataaggat |
| 19381 | agacatatag atcaatgtaa tggaattgag agtcttgaag taagcccata tatctatgac |
| 19441 | taattgattt tgacaagtgc gtaaaaattc attagggcat aaagagtctt tttgacaaat |
| 19501 | gatgctagga caattagata tccacatgca agagaatgaa gttggacccc taactcacgt |
| 19561 | catatacaaa acttagctca atatggatca atgacctaaa tataacaact aaaactaaat |
| 19621 | acacctagaa gaaaatttag tggtaatctt tatgaccttg gatttggcaa tggattctta |
| 19681 | catatgacat gaaaaaagta agccacaaaa gaaaaaatac ttaaattgga cttcacgcaa |
| 19741 | aataaaactt ttgtgtatca caagccactc caagaaagtg aaaagacgat gtatagactg |
| 19801 | ggagaaaata tttgcagatc atatatctgc gaagggctta aaattcagaa tatgtaaaga |
| 19861 | acctctacaa cccaacaaaa aatccaattt taaattgggc aaaagatctc aacggacatt |
| 19921 | tctccattga tttacaaatg tctagtaaac acataaaaag acgctaagca tcattagtca |
| 19981 | ttggggaaat gcaaaccaaa accacagtaa cRtattactt cacatccact agaatggcta |
| 20041 | tagtaaaaat cctggaaaat aacaagggtg ggcaaggatg tggaaaattt ggaactctcg |
| 20101 | tacattcttg gtgggaatgt acaacgataa atctactgtg gaaaacagtt tggtgactcc |
| 20161 | acaagaagct aagcatagga attgctgtat tatccagtaa tttcactcct agtatacatt |
| 20221 | gaaaacagga actcaaacag atacttatgt gccagtgtat attgcagcat aattcacaat |
| 20281 | agcccaaagg tagaaatcac ccaagtgttt ttcaactgat gaatatctaa acaaattgtg |
| 20341 | gtgtatacat acaataaaat attattcagt catacaaaag aatgatggtc tgatacatgt |
| 20401 | taaaatattg gtgagcctta aaaacatgct tagtaaacta aaccagacat aaaatgagaa |
| 20461 | aatctgtgtg attctactca tatgaattgc ctagactagg ccaattcata aagccaaaga |
| 20521 | ggagatcaga ggtcaccagg aactgtggga aaggaaaaat agggagttac tgcctaatga |
| 20581 | gtccagagtt tctatctgtg gtaatgtaaa agttttggac acagaggtga tgcttgcaca |
| 20641 | acattgtgaa tgtagttagt gccacagaat tgtacactta aaatggttaa aatgacaaat |
| 20701 | ttgttatata aatgttatca caataaaatt aaaacaaaat aaatgtgtaa attaaaaaat |
| 20761 | taatattcca gaccataaaa acatgcagca tggaaatgac tgtcattaga taagaacagg |
| 20821 | ttaaactttt aattttatat gaaatgaaca taaagatacc aattttaaac ccagcacatg |
| 20881 | aaaatttgat atcaatagaa acaggaaagg aaaaagatct gcaataaagc attaaaatag |
| 20941 | agtaaaaaga agaaatgaga agatagaaaa caatgcaatc acaattgtat tgagtgtaaa |
| 21001 | aatgtttctc aatagtaatt ttgagattac attattaaat aaagtgtcaa aaatgaaata |
| 21061 | tttgttttct acaagagaca tttaaaaata aaatgcagaa gtgttgaaaa tgcagaggtg |
| 21121 | gaaaacacac aatttagaaa tgataaatgc ttgaagtgat ggatcctaaa tatcttgact |
| 21181 | tttattacac attatataca tgaaacaaaa tatcacatat accttacaaa gttgaacaaa |
| 21241 | tattatgtat caaaataaaa taaaataaaa tgtaacaaaa ttagggaatt taccaagggt |
| 21301 | gaaataaaat ttaaagcaaa aattttcatg gcaaactttt ttaatatcag taaaaataac |
| 21361 | cataaaaaag taatgaccat gaataaaaat gcaattatca acattgcttc aaattaaatg |
| 21421 | caactacaaa taaaataatt atgagcataa agaaataaat cagtaattgt aagtgtaggt |
| 21481 | ttactaattt aactattgga aattgtcata tattaaagac gtaaacaagt aatatcatta |
| 21541 | atattgttca tctaatgcat atacatttaa ctttaacaca tcaaactcat ttaaagaaaa |
| 21601 | tcattcacaa aaatggatca tgtattgggc caaatgaatc ttatcctagg ccaaatatca |
| 21661 | tacacatatg ctgtctgact caaataaaat aagatatcaa tacaaaagtt acctttaaaa |
| 21721 | atctcattat tgttaaatat ctcttgcatt aaaaggaaag tgtaaagaat attttgaaat |
| 21781 | tatttgaatt gaatacaata taagaaccat gtgccaacat ttgtggaata catataagtt |
| 21841 | gatatttggg cagaaaacat acaattttga atacctttac taaaaaaaaa aagaaagatt |
| 21901 | ggacaaagct agtgaaacaa acaacttttg ccttcaaaag tatggcaaac cattatccta |
| 21961 | agcaaattaa cacagaaacc gaaaaccaac tatcacRtgc tcttgcttat aagcgggagc |
| 22021 | taaacattgg ttcacacaga cgtaaaaatg ggaacaatag acactggcga ctaagtggag |
| 22081 | agggagggat ggcaggggtc agggggcaag gactgtcaaa ctacctattg agtaccatgc |
| 22141 | tcactacctg ggtgatgggt tcaattatac cccaaacctc agcatcaagg aatataacct |
| 22201 | actaacaaac ctgcacctgt accccttaat ctaaaataga agttaaaatt atttttataa |
| 22261 | attaaaaaaa tctgtataaa aatatatgaa aaaccagatg tctggaaaaa tccttccagt |
| 22321 | caggaaaaca taaagatact gaaaaagaag ccttcagaaa tatttttaaa gtgtatggtc |
| 22381 | cagctgtcaa gagagttaaa aaagaatcct cagatgccaa aaatagtagg aaagtgcaaa |
| 22441 | tcatgagagg aaacagtgct gaagatagag ttagtcccta actaactatc ttggagttgg |
| 22501 | atagaggagt ttatcgagga gacggcattg tcactctcct ttaaaccagt gtgtaagtat |
| 22561 | tttttaattt tgttatgttt ttaaattgat gttccgcagt gaaaaggtat atgcaaaact |
| 22621 | atccctaaag gctgagggag ctgagaggcc aaagaaagag tctaacaatt ccagtttctc |
| 22681 | aaaaagaaat atttattgat ttatttataa tttttacttc aacaactttt tgggtacaag |
| 22741 | tgtttttttg tcacgtggat gagttatata gtggtgaatt ctgagatttt agtgcaccca |
| 22801 | tcacctgagt agtgtaaatt gtacctaata tgtagctttt ttatccctat cctcccttcc |
| 22861 | Rgcctccccc ttttgagtct ctaaagacca tgatatcact ctgtatgtct ttgtacactc |
| 22921 | atagcttagc tcccgcttat aactgaaaca tacagttttg gttttccagc catgtgtgtt |
| 22981 | acttcactta gaataatggc ctccaggtct atccaagttg ctgcaaaaga tatatatatc |
| 23041 | ttatacatat atatatctat cttatacata tatatatctt atacatatat atatcttata |
| 23101 | catatatatc ttatacatat atatacacac acaatctttt ttaaaagaaa gcaatattta |
| 23161 | ataggaactt ataaacagag gcaatgtctt gggtggccgc aagatattgg atccccacag |
| 23221 | ccgccctcca gaaaatatcc tttatatatt aagctttttg ggtaaaacat gtgcagctct |
| 23281 | tcatacctca ggctctcttg ctaaaactta tgaccattaa gaaggttaga taagcatctt |
| 23341 | tatgaggggc tctctatgtc acaggaatta tttaaagaac ttgctgcaga acacattggc |
| 23401 | atgcaggagt taaacatcag tcgttgtggt agtttcgctt caggatggca tcactctttc |
| 23461 | tacgcaataR gctattttcc tacgttatat aataattact gtacacattt atcaRgtaca |
| 23521 | tagtgatgtt ttgaaacata caatgtatag tgattcaata ggttaataag aagatctatc |
| 23581 | atcttaaacg tttatcattt ctttgttggg aatattaaaa tcctccttcc tggtatttaa |
| 23641 | aactatgtat tattgttagc tatagtcatc ctatagtcct ataaaacact agaacgtatt |
| 23701 | cctcctatct agctgtattt tgtatacttt tacaaacccc tatccttccc agcctctggt |
| 23761 | atactctgct cttcttttta cttctatgag atcttctttt tttagtttcc ttatatgagc |
| 23821 | gagaatatgt ggcgcttaac tttttgtttc tggcttattt caRgtaccat aatgtcttcc |
| 23881 | tttccatcca tgttgtagca aatgacattt cattttttaa aaaaaactgt tatggctaaa |
| 23941 | cagtattcca ttgtgtacat atacagcatt ttcttcatcc attcataaac cagtgcttat |
| 24001 | gttttaaaag accatatgca tgttggataa gatacctggt ttggacttct ccaaagttag |
| 24061 | aagtgagatt ggatactatc aacaagccaa gatccctgaa aggcaaaMcc ttaataggca |
| 24121 | aatgagaaaa taatataYgt ccaacaaaag gctaaagtag gaatacttaa cactcagcca |
| 24181 | gggcagtgaa ttgaaagggg aatgtaaagg ggaatgaaag aggaatgttg ctctaagaat |
| 24241 | ttgttataat agcctttcac tcatgtatat ttggggctag aagttgaatt ctacatagcc |
| 24301 | taaatcatta caaactgaaa agtttagctt aaagaagttg atactggaag tgatcccagt |
| 24361 | cagcctttac aaagtcaaac tttttctggc agacgtcact gtaaacctga tctcaaagaa |
| 24421 | ttcccacaga taatattcta ataaaactga gttcaaaata aaatcataaa acatgtgagg |
| 24481 | accaaaccaa tataattgaa agttagtaca aacaacaaag ttagtaatca gagctacaaa |
| 24541 | ggcagcagat atcagaatta gagcttctgc agtctgtgca aatgctaagg aaggttcttt |
| 24601 | ctctttccct tgtttctcaa agcaagtatg actaaacaga tattttttca caggcctcat |
| 24661 | cttttattta attatatttt tcatatgaca taaattattg catagaaaat tacataatac |
| 24721 | tacaagaaaa taaaaattat ctataatccc aatatctaca ggtaaagaac ctagacatcc |
| 24781 | agtttatgta taaatctaat caaaacagat tttttaagta acatgcttat tatctgtcat |
| 24841 | ctatctatgt tgagatctat tttacaacgt atgtagaaac atgttaacaa tggttagttg |
| 24901 | agtactgcaa atgtgcataa tcaatttctt ctttatatat ttttgtgttt gctaatttta |
| 24961 | gatgaaactg tattttatag tacaaaatgg agcttttgat gaaaggaaca ggtatcagta |
| 25021 | aaaataagct aatattggca aaaatagcag aagaaaacat aaatcaatat gagatcatgt |
| 25081 | aaattaccca cctattgccc caaacattac aaactagtta cttactaaac agcaaattaa |
| 25141 | gaaaaaatca taacacatat atggcatagt gttaatgtat aaactagtca caaaattggt |
| 25201 | aagaaaaact ataagacatg aaaaaataag gagaaaacaa atgaacaatt cccaaaaagt |
| 25261 | atatgctatt ggttaatccc gtagtaatgc ttaacttcac tagtaatcaa atatatgtga |
| 25321 | agtaaagata tttttgcaat caaagtagtg acattaaaaa taataacctg tttgattttg |
| 25381 | agactgcaac aggacggata ttttttcact attaacatat aggtttatta tatctacaga |
| 25441 | gcaatttatc aattctgcaa ggaatttatc aagtttctcc ccattggaat tttccttaaa |
| 25501 | aataatttac atactgctgt ctctatatat tagtcacact ggacctttag tttctggact |
| 25561 | accctctact ccttcttgct tcaaggcctt gacatgatgg tttcccaccc caaatttctc |
| 25621 | ctcttttgtc caccatactc ctacacatta ttaagatatc aaattaaacc atcttaacca |
| 25681 | cagtgacatc ttgtccattc tcatgaccag gtcaggtcag catcctatag tttttctctc |
| 25741 | ccaatatcat aaatatcatt gtttaagtta gctagctatt catttgtttt ctattagaat |
| 25801 | atattatcca ttatattggt atgttgttca ctactatatc ctcaatacct agccaagtta |
| 25861 | gcattcaaat agtattcata tgaatgaata gtattcactc tctataatac tcatattatg |
| 25921 | tttatttcta aaagtggaaa actgaagaca acttaaacgt aaacgatatg ataaacacta |
| 25981 | agtatattat tgtatatttg caaggggagt tattatgtac cccttaaaaa gattattaca |
| 26041 | aataattttt gaaaacatgg gaaaatgaat aaattctaca taatcaacat aatctataca |
| 26101 | gagaacatag ttctttatac aatataattt caaatgagcc aacctggtat atttggttga |
| 26161 | aataggcagc agcattaggt ttttttttct gttatctctt aactatctgt acattctaaa |
| 26221 | cacaatagtt taaaaatata aaaatattga tttggaaacc tgacatagaa aattaattaa |
| 26281 | gtccttatat ttggaaatat tccactcatt aggccataag tatgagatca aggcactttc |
| 26341 | ggaagttgcc aaaataagtc actgagttta taacctgggt tggacataaa agttaacttt |
| 26401 | aaaactcaaa atttgaaacg tttctgtgag tgcaagtttg tgtatatgaa tgtgtgtgtg |
| 26461 | tgcatgttYa tgtgtgtgtg tgtatgaagc ctgaagacag aacatttcat atttcaccca |
| 26521 | gattctatcc tgtgttcatc ttgtgtcctt tggtcaggga ctgtgttgaa tcttgcagcc |
| 26581 | tttggatcga tgcttctaga aaaatcttta ctcaacttta attggttcat aacctgcctt |
| 26641 | atctcaaatc actgattttg aagagcatat ttatttcatc agtgttttga tcccatatcc |
| 26701 | ctcaactaga cccttgtttc cctaactata tttcacagaa ctacatctac tcctctgact |
| 26761 | acttgctaca catcgtacac cagtaaaaaa ggatgttaga ttctaaaaaa tgaaaaaaaa |
| 26821 | acctttaaat aattaaaata gcattctgta aagaagtacc accatctatc ctcacttcca |
| 26881 | tgttctaaat gtcaagattg atctaatttt ctttctttct tttttttcct ttgggttttc |
| 26941 | tgggagaatg ggtgtccttg cttcagaaaa gtcaggtaaa gcacaacaag aagttttatt |
| 27001 | ttagagctta ttctcaccgt tataatcttt acagcgaata gtctccaggc tgatggtctt |
| 27061 | cctctaggaa gtttgtagga agaagttgca gggtggggaa gctgactaag ccactgaatt |
| 27121 | tgcatgtgag attaaaatct cctcaaagac tgagattccc tgaaacatag tcaaaacaat |
| 27181 | tatggaagat ttcagtgatc tttaatgaca cagttaaaaa gacaatgcaa gaatgcagtg |
| 27241 | caatgaaaca gatcttcaag gatttattca ctggattcct ttcgtggaac atccatctct |
| 27301 | tcccaaggtg tctatgtgac tctgagattt ttccttttct cacagcactt caattgctgc |
| 27361 | agtaccctca aagaggcttg ctttagcttg ctgtttccta gaattaagat aaatgagtga |
| 27421 | ccccagggat agatggctgt ggttgtcatt ccaaacataa gcagcagttt gttttctcgc |
| 27481 | acagtaaaac atgatatttc tatggccatg cctataaaat acaagataaa gaggatgatg |
| 27541 | aaagatatca aaactttcat tgccttcaca tgagcttctg tgttggagtc tctcaatcct |
| 27601 | gtcacattcg attgcatctg cctgttgtgt ctccaaaggg aaatgattaa aaaaatacat |
| 27661 | gtaattaggg atagtgtaaa gaagaaaatg actcccagat ttagcaaaat ctgtttaata |
| 27721 | aagtattcac ttttatacat gttgagatcc cagactgtgt cattcttcRt tttataatca |
| 27781 | ttaagaatct tcgcaatgta tgcaaaatta agtaacgatg aaataagtaa gaatactatc |
| 27841 | atgaagggaa gaaccatatt tgttctgctc ttcaaccaga gaaatatgta gttggaaaaa |
| 27901 | tttgctatct tcaggaaata gaagatgctg aggctggtgg caaaccacat acttgattga |
| 27961 | ttaccaatta cccaaaagta actaatatat tcaattaggt taccggaggc atatatattt |
| 28021 | ggagagaata tctgtataaa tccatctgta attattatcc atatcagaaa aattcttgaa |
| 28081 | atagctaagc cggtgagaat aaagccaatc gtagataact tattcttggc acagtcaatg |
| 28141 | cagtttacaa gtccaataaa tccattcccc aaaaccccaa acactgactc actaactaca |
| 28201 | acaaaaatga agatgccttc cactacacgt agcatatctg ctaattctta atattgcttc |
| 28261 | tgttacatct atcttagatt acctgctgca gaatgaggca tatattggct gctcgacgga |
| 28321 | agtgtgactt tcttctcttt cattatataa agacttgaaa ttgccccagt aatggcttga |
| 28381 | gatggaatca gaaatttctt aatgaagacc tttccaaata tgtctacaaa acatagctag |
| 28441 | tctggagaat gtatgttcag actgaatgcc tatctataat ttgttaaaat gcaaatgaaa |
| 28501 | gtttctgttt catggatttt acatgtcctc ctgagactat tttgcattta ataaattgaa |
| 28561 | ttgtggagga ggagtgccaa acatatacct atagaatgta atgcaaaaca gaaacaaaaa |
| 28621 | ttttgacttt atcttttaaa atatcaaatc attatttatg tcatttataa tcaatttata |
| 28681 | tatgtacaga aaatactata tagaaatgaa aaacaaattt agccactgta gatacttact |
| 28741 | ctaaatgatc gatttaattt ttaattaaaa tttaagctca actctaggta tgacaagttt |
| 28801 | cacaaatatg tcacaaatct gttgctgttg ttttatactt atggttatta cacccttgaa |
| 28861 | tgcagaagat tatgttgact acccttgaca tgaaaggctc ttaggaataa tctcttcatt |
| 28921 | taacacatta ctgtttgctg gcacccattc atttatccaa caagtattta ctgaatgtct |
| 28981 | agatacaaca agctctcttt tgatctttga gggaatatag tggtagacag aatttcaaaa |
| 29041 | cttgtgtgat ttatggagtg tatattctaa tgagggaggt gggaaacaat taagtaaatc |
| 29101 | aacatactga tctaagaatt aaaagtgaaa ttaaaagtga aatctaacat ttattgagct |
| 29161 | tttattatat tcctagcgct atgttaagta tctttttctc atcttaattt cacaagcctt |
| 29221 | caatccttta aaagaggggc tatttttgta tttacattaa atatgaataa actaagcttt |
| 29281 | ggagaaaaga agaccttctc tcacttatcc tggcatgact accataaata aaattgcaca |
| 29341 | atgatttcaa aatgaagtta aattaagctt aggaatagac tacaaatgat agcaaaacaa |
| 29401 | aacaaaccaa ccaaaagcaa gtagaaaact taaattctca tgggctgcac atactgaaat |
| 29461 | tgacagataa gaatatgtat agataaatta gctaatgtat acactgttta caaaaaacaa |
| 29521 | gatgcaattt cagagaagag cagcaagaga ctgttagaaa gaaattggaa catttgggga |
| 29581 | aaaaacttct agaaatggag aatataatta tataaaatgt aaatgaacta aatactccaa |
| 29641 | gtaaaaaatt aatagtaaca aagaagaatt taaaactctg acYgtatact gcttacaaga |
| 29701 | aacacaattt aaatatagaa aaggaaaact ttaaagtaaa attatggaaa aataagccat |
| 29761 | gcaaacatca tccaaaagaa atctggcata attataacaa tattctataa agcagacttt |
| 29821 | aaggtaagaa gcagcattag agaagaaatg gggagtgtac tttatagtga taaaaatgct |
| 29881 | caaatctaca gaaaggtagg gtaatcccaa aaatttatga accaaataat ctaccaaaca |
| 29941 | tttacagaaa aggaaaaata agacaaatct acaatcacaa tgaaagactg taaaatacat |
| 30001 | aactcaataa ctcatcttag aaacaccaaa aatcaataga tggaaaatac atgaaaaaac |
| 30061 | aattgacaaa cttgtgttca acggtaaaat aattcatatt cttttcaagt gcacatagac |
| 30121 | tatttttacc aagactgatc atgaatccaa aagaatatct cacaagtgtt gaatgttctc |
| 30181 | tgaattcaaa ggaataaaat gaagttcaat aataaaaaga taactagaga aagaaacaat |
| 30241 | ttactaagac aaaaaacctg taaatactct tgagtcaaag aagaaataac aaaaattaca |
| 30301 | aaaatatttt taagtaaata atacaggttg tgtatccctt atctgaaatg tgtgagacca |
| 30361 | gagagctttg gatttcggat ttttttttct ttttttttaa gttttggaac atttgcatca |
| 30421 | tactgattaa gcatctcgaa ttcaaaaatt tgaaatccaa aatgttccaa tgagcatatc |
| 30481 | ctttgagcgt Matgtcagcg ctcaaagagt ttcagatttt ggagcatttc aaatttcaga |
| 30541 | tttttggatt agggatactc aacgtgtaat aaagaaatga cctattaaaa tttacataat |
| 30601 | ccaattaaag ccattctttg aaggaaattt gtagccttga atgcatcttt tagaaaggca |
| 30661 | gggtaaaaac agatgaattt tgttttcatc tcaagaagct agaaagctaa gagtgaaaga |
| 30721 | aaatagaagt caagaaatta tagtWacagc agaaatcacc gatagaaaag aattaggtac |
| 30781 | agtaatatac taatgttggt caaaagaaaa tacaaattac tacagtcctt agagatatca |
| 30841 | gaaatatggc ctgggtgtgg tagctaatgc ctgtcatttc agcactttgg gaggcccagg |
| 30901 | tgtgagaatc acttgagtcc aggagtttga gaccagcctg ggaaacatag caagacctta |
| 30961 | tctctactaa aaaaaataaa taaataaaaa taatttagca gggcatggtg gtgtgcacct |
| 31021 | gtagtcccag ctacttggga ggccgagcag gaggattgcc tgagcccagg agtttgaact |
| 31081 | tgcagtgagc tataattggg ccactgcact ccagcctggg tgacagagtg agacctcatc |
| 31141 | tctctctctc tctttctctg tctctctctc tctctctcta tacatacata tatatacata |
| 31201 | tatatatata catatacata tatatatata tataatacta tataatatag ttacatatcc |
| 31261 | ttgtatataa tcttttatta taagacaata ctgtgaacaa ctttatacaa aatttcatca |
| 31321 | agaatactta taagactatt acaggctaat atagcctgaa catagatgca aaaattccaa |
| 31381 | acaaaataga gtccagcgat atataaaaga ataattcatt ataacaaaYg gagttttaaa |
| 31441 | agccaattgt gagattaaaa tgaaataatt gaagatacag tcaattaaca caaaagaaag |
| 31501 | ccagaaaaag gacagaggga caaaaataag tgagataaat gaaaaacatt gagcaaaatg |
| 31561 | caagacttac agccaaccat actgataatt acattaaaca tgatttgact aaacactctg |
| 31621 | atgaaaagat agagatcatc agaatgaata aatgaaagca caattaaatg ttatgcctag |
| 31681 | aagataaaac attaaacata aaggcacaga tagattgaat gtaaaggaaa gaaaaagcta |
| 31741 | tactatggaa tttgtaacca tagaaagcag gactggttSt attctcaaag tggctatatt |
| 31801 | atcaagacaa gtagtagaaa aatagaggga agttgtataa tgataaaata ttcaatttat |
| 31861 | caaaaatact taacaatcct gaaggtgtat cagactaata attgtgactc aaaataaata |
| 31921 | aagcaaaatt aacagaacta aagcaagtga taggcatagc aaagatttaa aaaccccttt |
| 31981 | gctgtgatct gcatgtgtct cctccaaagt ctatatgttg aaacttaatt gccagtgtgc |
| 32041 | cagtattaag agatggttcc tttaggaggt aatgagggca gacctctcat gaatgagatt |
| 32101 | aatgaccatt ttgatgtgca ttctaggtat gttaactcta catgccttag attgtgcaat |
| 32161 | agtaactggg ctcttatgaa agggttttaa caaMttccac aagttctaga ctaatttctg |
| 32221 | cagttacatc agcagcaaaa atattcacaa aaacaatggt gccactgcat ttcagcctgg |
| 32281 | gcgacagagt gagactccat ctcaaaacaa acaaacaaca aacaaaaaac ataaagtaac |
| 32341 | tggctaaatc catattatta cagagacaag attgttacct agaggaaata agtggttttc |
| 32401 | attctatttt gcttacttct gttaaatttc taacatccac Yttctgtaga tctRaatgRg |
| 32461 | tggattacaa cttattcaca cctctttttt atatataggt gattattttc tctttaaaat |
| 32521 | attttaaaaa ctattttaga ggtcagtcct ggcccaggaa gaatttattt attttatttt |
| 32581 | atttatttat ttatttattt atttatttat ttatttattt atttatttat ttttgagaca |
| 32641 | gagtctctct ctgtcgccca ggctggaatg cagtggcgcg atctcggctc actgcaagct |
| 32701 | acacctcctg agttcacgcc attctcctgc ctcagcctcc ccagtagctg gaactatggg |
| 32761 | cacccgccac cacgcctggc taattttttg tatttttagt agagacgggg tttcaccgtg |
| 32821 | ctagccagga tggtctcgat cttctgacct cgttatccgc ccacctcggc ctcccaaagt |
| 32881 | gctgggatta caggcctgag cccgcacccg gccaggccca ggaagaattt aaatggtact |
| 32941 | tactcagtga cacaggacat atacaaaaaa agagtacgat gaagcaagaa agattacacc |
| 33001 | aagacttctt gttgacttct ctctgaggcc tacctgtgta gctaagagtg cccgtccttt |
| 33061 | caggccacat ttcaggtgcc agagaatccc caagaaagtt ttcttcattt ggccgtatcc |
| 33121 | aaaaatcaca acatatgagt gacccaaaga atactgactt catatcataa accagatatc |
| 33181 | acaaccagtt tgttctgtag caaaaaatac ccctgtaaat gtcaagaaaa gggcaaattt |
| 33241 | ttatagagca acaagacaaa aaaggaaaac cgttttcttg gctctgaaat gggcccgtat |
| 33301 | actggggttc tgcaatccct gaaattgaac ctaatgtgtt tcatgtgtct tcacgaagaa |
| 33361 | aggagtaaca acaaaaagaa aatcgaagaa atagagaaag aagagagaga tccaataaag |
| 33421 | aagattatta tattattaac atttttatta ttacttacat tgagtaatcc agtcatatgt |
| 33481 | ctttcatatt tttttggaca cgataccaga agacatcaaa agtgtacgtc aaaggaaagt |
| 33541 | tggtaaacag aaaaggcaga taccagtgga agtatgaaag ctaccttgtt aattctccat |
| 33601 | tttagccaaa agtaaaagag ataggagaaa ttgacgatct ttaggaaata aaagacagca |
| 33661 | agacaagtag caaatcaagt gcacaagtgg ttggccagtc tccggacaat ggtaaggata |
| 33721 | tggtttcttt ccctagcatc agggattttc taatagaaga atattgattc tggctaaggc |
| 33781 | caaactagta agaatgaagc caaccatgca taacttctga tttctcatcc aggcaatgca |
| 33841 | atctgtgagt ccaatgaatt aatttcctac attcctttta taaatcccct acctattaca |
| 33901 | gtcatgaaaa cctccaatat attcaacatg gctacaaagc gaaaattctg atctgcagtt |
| 33961 | tgttgtgtga ctaattttca gatgacttac tggataataa catagatctt atgtttgatt |
| 34021 | gtgcagtgtt tttattttat catggctttt ctttttagct ataaagttta gcaaactgca |
| 34081 | gtaagacagt gaaatcactc tctgtaacat tcattgaaaa tttctaacta cctctgctaa |
| 34141 | gaatttccag tcttggtcca gctgtcagct acaaactgta aatttcttta tagaaagatg |
| 34201 | atcacactct ggggactgtt gtggggtggg gggaggggga gggatagcat cgggagatat |
| 34261 | acctaatgct agatgacgag ttagtgggtg cagtgcacca gcatggcaca tgtatacata |
| 34321 | tgtaactaac ctgcacaatg tgcacatgta ccctaaaact taaagtataa tttaaaaaat |
| 34381 | taaataaaaa taaaaaaata aaaacaaaat taaaaaaaaa gaaagatgat tgacaaacca |
| 34441 | aattatcatt tgctaacatg taaataaaga cacagactaa ttttcattgt tttgaataaa |
| 34501 | attagttctc ctcttccaat gaagtgtttc atttgattta caaatctgaa gtttgaaaca |
| 34561 | gaaacctttc accacattaa aattattcaa catgtaggga aaaccagcaa tccttatgct |
| 34621 | aattaattat aatgcagtca cagtgtagat tgctacatta caaagtaaaa tacagagctt |
| 34681 | gaaattttca agaaacttgg tgggcaatat atgaatctaa tatgagtatc ataattatca |
| 34741 | ggaaaacttc ccccaccgct ccccaccccc cactataaag atgcaggcct ttcaaataat |
| 34801 | ctgtcagtag cattttaaac acatttttaa aaatgatgtc tttcagtgaa acataagcac |
| 34861 | cccaaactaa acatggaaaa agtgaacttc ttttgtttcc tcaaaaacaa gattctagca |
| 34921 | ctttgggagg ctgaggcagg cggattgctt gggcccagga gtttgagacc agcttgggca |
| 34981 | acgtagcaaa accccgactc taccaaaaat acaaaaatta gctaggttag gtgtgttggc |
| 35041 | acacgcctct gatcccagat acacaggagg tggaggtggg aggatcacct gagcccaggg |
| 35101 | aggttgaggc tgtagtgagc cacgaaaaaa aaaatgtttt ttttgagata Waagagtctt |
| 35161 | caaatatttc cacagaactt tgttaaggtt acattgtctt gaggggaaaa tgcttaaaaa |
| 35221 | gtcatgaata tacttaggat gatctgttat gtgtatatcc atatacacag atataggtac |
| 35281 | atatatggct acatatacat aaacatatac atgtatacaa atacacacag acacatctat |
| 35341 | ttatacacac attgtactgt gWtttgtcct ctacttctct gtattttagc ttgtctggtt |
| 35401 | tttgttactt ggattgcttc tttttttgca ttattttatt atttttaata aacgcaataa |
| 35461 | ttgtacatat ttatgtgata cagtgtgata tgtgaataca tttatacaat gtgtaatgat |
| 35521 | caaatcaagg caattagcat atccgtttct caaatattta acatttcttc gtgttgggca |
| 35581 | cattcaaaat ttgtttttct agctcttgta aaatatacaa taaattgttS taattgtagt |
| 35641 | caccctactg tgctatagaa cactagaact tgttccttct acctggctat acttttgtat |
| 35701 | tcattaacca acctttggct atctccctct cccttctccc ctatcacatc tctaggaacc |
| 35761 | attattctgc ttcctacttc tacggaatca actttattag cttccacata tgtgtgaaca |
| 35821 | tgtcatgttt gtctttctgt atctggctta tttcacttaa cagatgttct ccagtctcat |
| 35881 | ggatgcttcc ataaatgacc aaatttcctt cttttaatgg ctaaatagta ttccattcat |
| 35941 | tatgtgtata tatcatgttt tctaatccat tcaaatgtta atggacactt aggttgattt |
| 36001 | catgtctcgg ctattatgaa tggtgttgca aaacacatgg gagtgcagat atcacttcga |
| 36061 | catattgatt tcctttcctt tggatacata cccagtggtg ggattgctga atcatatggt |
| 36121 | agttatattt ttagttttta agaacttcca ttctgttctc cataatgact aaactaattt |
| 36181 | atatttgtac caacagtgtg taaaacttcc tcctccacat cctcacaagc atttgtgttt |
| 36241 | ttttgtcttt ttgaaaatag ccattataac tagaataaaa taatatctca ttgaggtttt |
| 36301 | tatttgcatt tctctgatga ttagttatat tgagcacctt tttatatacc tattggccat |
| 36361 | ttgtatgtct tcttttgaga gatatatata cagttaattt gcccatgttt taatctaatt |
| 36421 | atttgtgttt ttgactgagt tccttctata gtctggatat gaattccttg ataaatgaat |
| 36481 | agttttcaaa tattttctct tattctagag gttgtatcgt cagtctgtcg attgtatatt |
| 36541 | tgtgtatgct tttatcaaat caacataatg taaactaaat gagataaagc taaaatggtc |
| 36601 | atcgcttgct taacataaaa tgcattattc atctaatttt attgttacta ttatataata |
| 36661 | ctataatttt aattttgcca ttgattatat agttgcaatt ctgatggaaa cagtgaaatc |
| 36721 | actttctttt aacttatttc aggcagataa tttcatcaat gaggctcaat gatctatcac |
| 36781 | caaacataat aaaagaagtt ggattaaatg aaagggatgt gtgctgagtt atttgttgtc |
| 36841 | ttcatttttg agagaattac caatcaattc aggtgtgttt atgaggattt agatttcctt |
| 36901 | atattcatgt aacaccgaag gttcctcagS acacagagag aagcctgttt cagtttgctg |
| 36961 | ttccataaaa tcagaaaaaa atgaatgact gaatagatag aaaaatgcta actcaagagc |
| 37021 | aaaaatctgt gccacaatgc tatctagatt aaaataattt cgctttataa tcgtattgct |
| 37081 | caaatagtat ataataaaga ggagcaggaa tgaaattata gatattaggg gatgtcaaac |
| 37141 | ttatccttgc attcttattg tacaaaattt cctcagttta catttaagcc cacatgtaaa |
| 37201 | attacagcag aatattctgg aaattgccca acaggtgaga ataaaatcta tcagggagag |
| 37261 | tttccagcgc ctgatccaat caatgcagtg aaccagcacc ataactctac ttctctgaat |
| 37321 | ccccattatg aaatctatag cagaaatcat tgtaagcata ttcttcaaca tgtttgccat |
| 37381 | gcttgattat gggatctctg ctttctgtac accactgatt tataaataag atgttataaa |
| 37441 | tttgtgtatc caaagcttta gtgttcaata tgtttcttta tcctgtgctR ttgattgaca |
| 37501 | ctggaaaaga atggaatgcc tccagtgtgc tcccagacaa agtcctagtt gtctttgaaa |
| 37561 | aacgaaaact acctttattt ttctcagaat ttcagctagt gattttcaag gcagatatta |
| 37621 | tattacctgc tgttgtgaca ctgtttataa tttccttgta aagctatcaa ctaggatttg |
| 37681 | aattctgatt tgttaaaata caaaatagag attttaaaat attttttgca tctttttttc |
| 37741 | cttaagattt aaatgttttt tattcattca ccttagttat aagctagaaa cattaaaata |
| 37801 | tggtgtcttc atggatctcc tgagaagcaa ggcagtcatt gggataaatg cctatatgag |
| 37861 | aaagcaagga gagagcgaaa ggtgaacaga gctattgtac taggatataa ctttgacccc |
| 37921 | tgtgaaggag agagagaggg aaagaaggaa ggtcttaggc agcagtgcag tcctgagaac |
| 37981 | atttcaagat gtccagtagg gagttatcaa gcccaaggtg caaattagag gagtcctgag |
| 38041 | tctcccagga gcaggcctga ccattgaatc tgcccccttc tctgtctttg gctgaaagcg |
| 38101 | gctcatggaa gtgaatgggg tgatagattt catagcatag gtgccatgct tgttagctgt |
| 38161 | tggggaaata cgattaaaaa caaagtctcc tcccaaccca gaaagtctct ccacaaatgt |
| 38221 | aatagagaaa gaaaatagtt ttcttattaa ataaatatta aaccagaatc tgatatacat |
| 38281 | tacaggcaac ctactaagag actgcaaaga cagaaagaga tctcacccgt ttttatatag |
| 38341 | ccaatatatg catatgaccc aatatataca ttttctcaag ttaagtgata actaatcccc |
| 38401 | aagagagagg acttggcagc attatttatt aYgcatagtt tatcttaaat ttacctggaa |
| 38461 | attggggtgg ccatctgtgt tagttaacag actttaaaca aaggacaaat aaacacagat |
| 38521 | ctttatgtag gagatagtct tgccatttgg agcctggtgg ccattgaagt taggctcctg |
| 38581 | ctcccagcag aaactgagag ataagggtgc ttatttgtaa aagagatgat taagcttctt |
| 38641 | tatttttagt gtgtatttgc ccttctgcag ctaattgtgc tccctcaRga cagagatttc |
| 38701 | agaggcacat tcctatcaca tgccacaaat gtctaaaatg acagtttatc ttcttactca |
| 38761 | cctcgccaat acatattatg tataaagtac ccaaggttta agtaaataca caaaatctgc |
| 38821 | tttctgatac tatatcagga tcgcttagag tctgtggcag ttttgaaaca tggctgcaaa |
| 38881 | ctctttgatc ctcttccttt tgaggcctag ggtctgcaga ccttcctctt aaaactgagt |
| 38941 | gggcttgtaa ctgctttaaa caatgacgtg tggtggaaac gacatcatat actttctaag |
| 39001 | gctatcaaga aaaactgtat ggcttttccg ttactcactg gaaagtttcc tcttacaatc |
| 39061 | ctgaaccact tcataagaag tctgacaaag ttgagaccag catgctgtga ggaagctcaa |
| 39121 | gccacacgag gaggccacat gtaggcaata tagtcaacca tttcaaatga gcccgtactt |
| 39181 | ctgatcatcc aagtccagtg atagacatga gagtaaaggt ctgcttggtt attgcagccc |
| 39241 | ctgccacttg agtcatagtc cgctgtctac atattctaag gtgaaggtct agacattttg |
| 39301 | aagaagagac aagccttctc atagtgccct gtctgaatta ctgacccaca gaatctggaa |
| 39361 | gcataataaa gtactgttgt tttacaccca ctagggccta ttggggtggg ggttggggga |
| 39421 | ggaggaacat caggaagaat agctagtaga tgttgggctt aatacctagg tgatgggctg |
| 39481 | atctgtgcat cagaccacca tggcacatgt ttaccaatgt aacYgcacat tctgcacatg |
| 39541 | tacttcagaa cttaaaagtt gaagaaaaaa tacacaaaat aaaataaaat aaaatattgt |
| 39601 | tgttttgtat ctctgagttt tggactcagg gtgttttaaa gatcaatgca taactaaaat |
| 39661 | agaattattt agcacataca taaggaaagt cttttactag cttagttttt attttcttag |
| 39721 | catcattatt tatagtagcc aaaagataga aaaaaaccca aatgttcatc aatgaataaa |
| 39781 | taaatagaca aaaggtggtt tagtcatgta atcaaatatt atttaactat taaagggaat |
| 39841 | aaagtactga tacgtacaac atgtatgagt cttgaaatta tgctaagtaa aagaaaccaa |
| 39901 | tcacaaaaga tcccatatta tataatgcca ttcagtcgaa agtccagaat aggaaaatat |
| 39961 | acagatgcag gaagaagatt aatagatgct taggctgggg tggggagaaa ggtgggatga |
| 40021 | tgggggaata gggaggtgat atctaatgga tatggggttt ctttttgaag tgataaaatg |
| 40081 | ttctacaatt gacagtggtg atggttgcac atgtctgtgg aatatattaa aaatcattta |
| 40141 | attgaacact aaatgagtga attgtatatt atgtgaacta tatctcaatt aaagcattta |
| 40201 | aggcaaaaac aagttacata tgacattttt ccttatagaa tatgtaacat ctgtgtatat |
| 40261 | ttttcataat tttattatac cttgtagtaa cactattagc tcagtcatct ttaattctgg |
| 40321 | tgaggtataa ataaaatacc aaaagcttta catcatttca cttttgttga ctattactta |
| 40381 | ttgagaaact ttttactctt tggatgagac atgataggat aggctatgct gtagcatcta |
| 40441 | caatgtaagt ttcatgaaag gttcatttct cggttcacat tacatgacta attgaattta |
| 40501 | aaaatgcaga gtgaggtgag gatagttttc atgtagtctc tcaggaatac aagctagtgg |
| 40561 | agtatctaac actaaacatg tgtctttcaa gtccctcact taggaagaaa agagacacga |
| 40621 | agagccttct tttacatatg aatattcaac tgttccaaaa tgattagttg aaaagataat |
| 40681 | cctaatcttt ttttttcagt ctttaagtgc tcttttcttt tattctcagc cccctttatt |
| 40741 | gttattatta ttttcatttc attattatta tactttaagt tctagggtac atgtgcacaa |
| 40801 | cgtgcaggtt tgttacatat gtatacatgt gccatgttgg tgtgctgcac ccattaactc |
| 40861 | gtcagatttt atgtttcttt atcctggctt gataaaaccc attgtctcag gacttttctt |
| 40921 | ctaaatagaa aagtagtctg aaggctcgtc ctgattcatt ttaagactgt agtatttctc |
| 40981 | ttctgccagt taatatgttt tcactttttc tattttcttt ttatatccaa tacatttcag |
| 41041 | tatgaaattt tgtgatggtc cactagtctg atgtgcacaa tttagccagg aataaatatt |
| 41101 | agtggattca aaataggcac tcaaactatc tgtccagatt ttttaagtgt tgttggtttt |
| 41161 | tttctttctt tttttttttt tttcaaattt caactgatga atgaaagtag ctgcctagag |
| 41221 | aataatgagg tgggtgccaa acctgggttc attgaaggcg ttatagcacc tgtgccatgc |
| 41281 | tataacttag ataagtgacc ttatctaagc ccatggcttc aaatgccact gaaactggtg |
| 41341 | aatttatctt ttaggaatgg aagggctata gagatatttg atgaaagaaa actaagagga |
| 41401 | tttgtcacca gaacatctac cttttaaaag ggggctaaag aaaattctct agccaagaaa |
| 41461 | aataaaaaag aaagtttcaa acaaaaagat taaagaaaac acaatggaaa ggaagaatga |
| 41521 | ataaattcat cttccttttc ttcttcagtt tggtaaatgt atttgacagt tgaagttacM |
| 41581 | attatgacat tatcagctgt gattgtaaac ttgatttcag taacatttga agccatgggc |
| 41641 | ttagacaagg tcacttagga atagtgtaga tatcaggaca agtaagacct ctgagacagc |
| 41701 | catgtgggaa ctcgacatag caagcaagcg atggggagcc ccacccagtg actgtcttca |
| 41761 | ctctagcagc tctcagtgaa ggcttctgta ttaggccatt cttgtgttgc cataaagaaa |
| 41821 | tagaggggcc ctgcatggtg gctcacacct gtaatcccag cactttgaga ggcagaggcg |
| 41881 | ggcggatcac gaagtcagga gattgagacc atcctggcca acacggtgaa accctgtctc |
| 41941 | tactaaaaat acaaaaaatt agccgggcgt ggtggcgggt gcctgtagtc ccacctactc |
| 42001 | agcaggcaga ggcaggagaa tcacttgaag ccgggaggtg gaggttgcag tgagccaaga |
| 42061 | tcgcgccact gcactccagc ctgatgacag agcgagactc catctcaaaa aaaataaaaa |
| 42121 | aataaaaaat aaaggtaatt aataaaagag atttaattag ctcatagttc tgcagggtgt |
| 42181 | acaggcatgg cactagtatc tgattagctt ctgatgaggc ctctggaagc ttacaatcat |
| 42241 | ggcaagaggc aaagggggag gaggcgtgtc acatggtgag agtgggagca agtagaggag |
| 42301 | gcgccacaca cattcaaaca accagatctt gcatgaactc agagcaagaa ctcactaatc |
| 42361 | accgagagaa tggtgttaaa ccaggagtgt ccaatctttt ggcttcccta ggccacgccg |
| 42421 | gaagaaaaag aattgtcttg ggccatacaa aaaaatatac taacgatggg tgatgagcta |
| 42481 | aaacaaattg ccaaaaaact caatgtttta agaaagttta tgaatttgta Ytgggtcata |
| 42541 | ttcaaagcca ttctgggctg catgcagaat gctggccatg tgttggacaa gcttctgcta |
| 42601 | caccattcat gaggaatctg ccccatgatc caatcacctc ctaccaggtg ctatcacaac |
| 42661 | atcgRgaatc acatttcaac atgaggtttt caggggacaa acatccaaac catatcagct |
| 42721 | tccctgtgcc aagcatggat ggagctgaga gctttatatg ggttacttac aaaatataca |
| 42781 | tgttttatac aataatatta atagtacaaa ataattactt tgtaccaaaa attaattgta |
| 42841 | caaaatatta aaatatttaa cataacattt gtacaatgta tataaaaata taaatatccc |
| 42901 | ataaaatttg ataatagcat atgctatttt atttctataa tatagacact cttcaatact |
| 42961 | cagcaacttt gttcacatta gaccactttt atttgtaatt attgatatgt tgggtcacaa |
| 43021 | acctgccatt ttactttRtg atttctgctt atccgttttt ttttcttttt ttgtaattgt |
| 43081 | tgttttgtac gccttgcttt catgtgtgtt actttaattt tcttttttta gaattccatt |
| 43141 | gaggtttatc tgtggtattt ttgagtatat ctctgtgtgc agacatctta gtggatgctg |
| 43201 | taggttttac agcacatgta ataacttatc atggtttaat agggtcaaaa ttttagtact |
| 43261 | ttgaaagatg tacggaaacc ttattccttg attccctata tgtctcttta ccctcctctg |
| 43321 | ttccttatat agttgtctta aatatttacc tatatacact taaaatcgca tctgataatg |
| 43381 | tcataatttt aacttcagct gtcaaataaa tttaccaaac tgaagaggaa aaggaagatg |
| 43441 | aagttattca ttttttccct ttccattgtg ttttctttaa ttttggtgtt ttaaagtttc |
| 43501 | ttttttaatc ttttttcttt ttttttttcc tggatagaga attttcttga gccccttttt |
| 43561 | aaaaggtaga ccttctggtg acaaatcctc tcagctttct ttcatcaaat gtctttatag |
| 43621 | cccttccatt cctaaaagac aaatatacca gatataaaaa ccagaattga aagttatttt |
| 43681 | atttcatcac atagacagtg ttgtgccact tttctatggc tttcatctat ttctgatgtt |
| 43741 | tcctgcttcc atttgtattg ttttctctcc tatagatgag ctatttcctc tagctgcttt |
| 43801 | caaaatttgt tctttctttt aattttcaga tattttacca tggtatacct tagtttggat |
| 43861 | tctttgtgtt tgtcttggtt agttttatgc actgaaagtt tgtgtcccct caaaattcat |
| 43921 | atgtagacat cctaaattca atgtgatgtt atttggaggt taggcctttg ggaggtaatt |
| 43981 | agggttagat taagtcatga gggtggggcc ctcatgttga gatcagaggc cttataaaaa |
| 44041 | aaaaaaagag agagagagag ggagaaggag acaagagata tctttttctt ttctccaaca |
| 44101 | tgtgaggata caatgagatg atgtccatct atagccaaaa aatgtcttca gcagaactca |
| 44161 | tctgtgctgg cattctgatc acRgtcttcc agcctccagt attgtgagca acacattttt |
| 44221 | gtttttataa accacccatt ctatggtatt ctgatacagt agcctaaaat ggctaagaga |
| 44281 | ttttgggttt gcttgaattt aggaatctgt gggtttttgc ctattgccaa attaaggaag |
| 44341 | ttttcagtct ttgagacctt tctcaaagtt gcccatttct tctctccttc tgggactctg |
| 44401 | ataacacaaa aattaggcct tcagttataa tactacaggt ccctgaggct gtgtttatag |
| 44461 | ttttttcctg cttaccttca tgaatttttt atgtggttat tgtaagtttc agttctaaaa |
| 44521 | tgtccattca gttctttcat atattttaat tttttggctg agataatttt taaatttgtt |
| 44581 | tcatatatgt ttgtaagcaa tatactggaa gcatttttat gatgactgct ttaaaatatt |
| 44641 | ttctgaataa ttccaacatc tttgtcattt ttgttctgga atcttctaat taccttttac |
| 44701 | attcaagttg agatttttcc tggttctttg tataccaaat gatttctaaa tgaaccagga |
| 44761 | cattttaggc attatgagac tctagaactt atttaccttt tctgtcttag ctagcttcct |
| 44821 | cagacactac tccagcaagg caagggagtg agggagggac actgcattac tgccaggtga |
| 44881 | tgatagaaat aaaggtttcc tctcaatctc tgttgacacc taggcaggcg gaggttcctc |
| 44941 | attactgctg gctggagggt ggattctggg tcaccactgg gcctccactg atagcctgtc |
| 45001 | tcggaggaag aagtgacttg ttactgtttc ccacatggcc tccacaagca ccacctggta |
| 45061 | tggaaaggag ggtgcttctt cattgctgct tttagagtag atccagctcc ccatttgctc |
| 45121 | tccagtcata tcaaggtgtg gaaggagagt gaggggctta ttaccacaag ttggggatga |
| 45181 | aagttccaac ttctgctatt ctgagatact attctggagg agggttggga caccttgtca |
| 45241 | cagcctggct ggtatgaaga tctaaagatc taagctcttc acttgaggta atagtgtttt |
| 45301 | ctgtggtact tgactacagt agagtggtta ttgtcgaaat gttttctgcc ttggtagact |
| 45361 | atttccttga cctttgccta aaataatagg cttttgttgg gaaattctca tatgtgcctg |
| 45421 | ttagtatttc tatgttgatg gcattttcag ctaaaagtct gcaaattata aagcagaaaa |
| 45481 | gaaaacttag aggattcatc actatacaat ttattaagtc cctaccacat ctgccttggt |
| 45541 | ctctccaggt ctgagttttc atatatttta tatataatgt tcagggttta tagtttttct |
| 45601 | tagtagaagg aatagggcaa aatacattta ctccattttt ctggaagcag aaattggttt |
| 45661 | actttctgta agaaagcagt tggtgaattt ctgtctaaat tctctgcctt ttcaagagga |
| 45721 | actgggcttt acttctcaga taacaactga gcaacgatag acctacaggt ggcattactc |
| 45781 | tctgtatagg tagagaatag gctgaatatt aaatgttaag gagcagaatc attggaattt |
| 45841 | aaatgaggaa ggagacttag tttttattca cgttaaccag ccacctgatg acatgcaaca |
| 45901 | gtgcgtttgt tcatcaacat accttttaga tggcctttct gcttcttttt tgaatggctt |
| 45961 | taatgctaaa gaaagcaagt gtttatcaaa taatctattt tttagataat tcattattag |
| 46021 | aagttaattc attattagaa agataattta ttattagaag gtaattcatt attagaaaga |
| 46081 | acaaaacgag tcttttttaa taaaagaata aataaattta aatgtattga acttttattg |
| 46141 | ttgtatgtca tgtagggtaa ttttgtatac atgttgcatt ttacaagact taaatcgtgt |
| 46201 | aattttatat acatcttgta gggtaatttt atatacattt tataaggtaa ttttatatat |
| 46261 | attatgcatt Waattcttgt aagtagccct ctgccttagc cagttcgagc tgctattaac |
| 46321 | aaaatatctt atcttgggta atttataaat aatataaatg gatttgtcat agctctggag |
| 46381 | gctggaaagt tgaagattga gatggtacca gatttggggt tgagtgagag cccattccaa |
| 46441 | acagacaatt tctcctggct tctctctcat gaggcagaag tatgaaagaa acatgaacat |
| 46501 | tgagtcccta catgtcaaaa gagatggaac agctcagcag cctttgggag cttctttttg |
| 46561 | tagggaaatt gatcccattc atgaaagcag agtccacacg acttagtcac ttcccccaaa |
| 46621 | gactccatct cttaatacta ttgcataggg aattaagttt caacataaat ttattttgta |
| 46681 | gagacataaa cattcaaact atagcattct gtccctggtc atcccccgaa atatatggtc |
| 46741 | ttctcacata ctaagtacat ttatttcatc tcccaaatcc caaagtctta actcattcca |
| 46801 | gcatccactt taaaatctaa atccaaagtc ttatctaaat atcatcaaaa tcagatatgg |
| 46861 | tgtggccaat tttctaatat tgaaccatct ttaacatcct catcctcttt caacgtggtt |
| 46921 | tattattctt ttagcatact gttcgtttgt ttatgtacta ttgaatagtt tccctaacag |
| 46981 | agtatccttg ctgtcctata tattttgttt aatgaaactc agaatgaatt cagaaacttg |
| 47041 | ttgtttcctg tacttaaaaa atctaaaaaa gaattatgtg ctttttaatg ataaagatta |
| 47101 | gatattaaaa ttttctgtat tcagcgacat tggaggtgat aatgagaggt agctggagac |
| 47161 | agttacatat caaattcttt tgcttcttag ggttattttc tattcaactt tgcctcagca |
| 47221 | acttgagtta aatttggtag tctatattat ctgacaataa tactgatatt gctggaattt |
| 47281 | ttacaatcag aacagagttt tactcaaatt tcttgccctt taaagtacat tatgctgcac |
| 47341 | cctttatgaa atccactcaa gtttttaaaa agaatggtga acgacttagg gaggaagaga |
| 47401 | gctgagcaat gaaaaaagag gagaaaagag catagtgagt tttatagaga agagatctag |
| 47461 | aactacattt atgcccctag aaaccaggat taatagtttt ggaaatttta agaattcccc |
| 47521 | agcacttgca actccctctc atttgttata tattccttgt tttcctagag ttacaagaat |
| 47581 | atcaatgaaa gatgtaaaaa taattttaag aatttgcatt tgtgctatgc aatctaggac |
| 47641 | agaaggggaa caatggaact ctttctcctt ggcagctggc tttattaaga taaaggagga |
| 47701 | gggaagcctg ctggaagcat caatgagcaa gagtcccaga tattcttaga ttgtattaat |
| 47761 | ggccctcaaa ttctagactc agaaacaccc acaaagagaa gcaacaatca gaaattgcat |
| 47821 | gctattaata ttttattggt atactgaaga aagagttatg accacattat ttcaatgtca |
| 47881 | tggctttctg aaggaagtta tcttcttatt tattttctga aacaaaaaaa aaaaccaagg |
| 47941 | aaatttatac acaacataca acatggcaaa acttatctaa ttccttgatg atattactga |
| 48001 | agtaaattat tgctttcaag attaagaaaa cagatttttt tatggtgttt atgttttggt |
| 48061 | tactgttcaa aacattcaag gttatattta tgaaaatgac tgcaacttga acctgtagta |
| 48121 | gtgaaaaact aatacaagcc tacatatcaa ttaacagtgg ttgactgagg aaatcatggt |
| 48181 | acagtcacac aagagaatag tatacaattg taacaacaaa agaaaaatgt acaaataaat |
| 48241 | caccaagaag atctataggt ctccgtgaaa tgataaagtt gtctaagaca tgtattattt |
| 48301 | gaaataagca aatacagatc tgcatataaa ataatatgtc atttgtggga aaaataatga |
| 48361 | cgtatgctta tatacatata aatatttcca ggaggatgtt caaaaaatta agggtggtga |
| 48421 | cttttggtga agggtgtggg taagtaggat aaggcgtcac tttaagattt agcttcatac |
| 48481 | cattctgttc agttagattt ttttttcctt gagcatgtac taatttcaaa ataagtattt |
| 48541 | taaaaattaa ataagatata agagcatcaa gttcaagtat catttagcag ggacactgga |
| 48601 | cctgagaggc tagtgtgagg caggactgag caaagagtcc tctggcggaa acctgagaga |
| 48661 | aacctgagat ggagagtcct tgggtttgag aaggcagact caaagacagg aggcagagca |
| 48721 | ccaaagggag agcacacccc acttcctcat taccaactca ttttaatgag acaagtcatc |
| 48781 | ttaagaaaaa agttggggag gtcagtattt caaaagataa gaattctttg aaccacttgt |
| 48841 | taaaaataca aggacttgca accttatcca agaatatctg aatacaaatc tttagaaatg |
| 48901 | gggtccagga ttatgtaatt tcaacatatt tccaattgat ttgtgaacac gataaagttt |
| 48961 | gggaatggta gcagtttggg catttaatgg agaatgaact ggaatcatac ctgccactga |
| 49021 | attctagatt accagagtgg ttgctcctgg ggatgtcttg ctggtctgtc cctcYggaag |
| 49081 | aatgatgatg cttcctgcag gctgacagaa ggaaatcggg gtagagagag ttgacggtgt |
| 49141 | cctcgtYggg gtggtcgttg ctgattttga aaaggaggtS ggggaggatg gcggtgatgg |
| 49201 | cctcctggtt ttggtggtct ctgctgagga ccatcatctt ggttaccact atcaccaggg |
| 49261 | ggtctaggta ggagtccttc aggaggaggt ctctggggtc cctgatctgg tctctgactt |
| 49321 | gagtcctcta catctgtgtg agtaattaat ggacaagaat gcatgagctc agtgaagaaa |
| 49381 | aactctcctc tctttatact tctctcacct taccacacag ccctgttctc cccaaacctg |
| 49441 | ctgcttgact gtcaacttct tttgtgccca ctgttttttt tttcttttta tgcagctctc |
| 49501 | aacttaatcc atgtaagagt gatatggaaa gtctcattta tttgctcctg tgacaagtat |
| 49561 | gtttccttaa atgaaaagag gggttcttgc atatcttaga gttgccagag gatgtctcca |
| 49621 | aggacagaaa aaaaaaaaaa agcaaaagca ttatagcttt cagttatgtt actggttatt |
| 49681 | ttttattttt attttttggt tgctgtaatg acttacaaac tgtgactcca tctctgggca |
| 49741 | cctaagccct atgtggaaac tagagctgat ttcccaggct cctggaacca agtagagaaa |
| 49801 | tactaagaga cagaagagaa agacaatgtg aaagcctgca catgatcact aaacagctgt |
| 49861 | ggcaactatt tatcatctgt agtgacacag ctgagtgggt caacacagag aaaagacatt |
| 49921 | catgcagtga tttttttgtt ttgttttgtg ttactccctg aagccacata accgtgcggg |
| 49981 | ggaacactgg gggaagaaag aagcagttaa ggatggagga gtcagaatga gaacacttct |
| 50041 | catttgttca tcatctctca gcagtcattc cagcctggga aaaaagcctg cccactccta |
| 50101 | ttgtcctcaa agccaggcat ctctgtattc aagttggtgg cctggctcat ggtccccaga |
| 50161 | atcaaagatg ggtaagaatt gcctatatat ttaggggcac taatattaat caatgcctga |
| 50221 | cccctcttac cacctcctcc caaaaaaatg atgagaagaa gacactgaag gaaactaaaa |
| 50281 | agaaattcta gtggaaaaag Raagaaaaga attggattga ggatcattgg gatttacctg |
| 50341 | gtatggtgaa agtaaagtct tcatagttca catctaggaa aagaagcaca ggatgaagtg |
| 50401 | aacattacct cataaatctt tcagggctca tagtggtcta tagggaaagg ggtcttctgg |
| 50461 | ccacaccctg tgcatcccct aagttacctc atcaaccatg ttctgtgaag ctgctggaag |
| 50521 | gggaggaaga tgttagggga ggcaggattg ttactatcgc tgagcatcaa ccacgaattc |
| 50581 | aatagaagag tccctttttg tcatccttag gatccctcaa accctaatgc taatttaggc |
| 50641 | acacttctct gggtattttc atgtcatatt ttagatcttt accttcttcc ttctttaaat |
| 50701 | attttgatca tattcacaag cttgcccaag aggagcaacc aggagtaaat atttggatat |
| 50761 | aatcttacac atRctgcctg caacatattg aaattttaaa tgtgattgga agaaaaatgg |
| 50821 | gaatttgggg acctgatatt tctataccac tatgtagaaa gacacaaatg tattatccag |
| 50881 | tcctctatgt ctgacagtac tctgatgttt gtttatctct ttcattgatt ctgcatcccc |
| 50941 | tgtacagcaa ggacaccatc atcgctgtcc actgagatta ctgtagcatc ttcatagctg |
| 51001 | gacttccctg gatcagtctt gcattctcca ctgcatcctt cacagcacag ccaggtcttt |
| 51061 | ctaaaaatac aaatcttact ttgtcattcc tctaattgaa attctctatt gggttccaag |
| 51121 | tgtatatcaa ataaagtata aggccttggc atggaatgag ggcacaacag atgtcatcct |
| 51181 | tggaatgaga gctccaaagc cctacccttc ttctcaacct ccttcctccc ctcctgctct |
| 51241 | tcYgctgcag tggtgtaggg tgaacccctg aggccctaga agagtcatgg cccctcatcc |
| 51301 | cactccccat ctcctttttt aaaaaaataa tttttttacc attatctgtg ctctgagctg |
| 51361 | agctcagagc cagaaggacc actgagagca ggaccagcag catcttgaag gaggctctgg |
| 51421 | agttgctccc aactctgcgt tgagagaaac atggcagctc cctttataaa catgagcaga |
| 51481 | acaatggcac ctttgagctc catactgggt ggacctcacc acctgaaagR ttaggttttg |
| 51541 | ctttcctctc ataaagttga atgcgtctct tattactttt tgggatttta gcccagcagg |
| 51601 | atgtggtggt aggatgtggt taggtacaaa ctgtgacttg agcatagttt ctgaagaaac |
| 51661 | agtatccaag caatcagcac aaatactaca attgaagttt ggtcatcatt tgattttcaa |
| 51721 | ttgggttaga cagttagtct ttccccttca ttggttgtct gtgtgcacac gtgcttacgt |
| 51781 | ttgtatatgt ttgtcagcag tgatcttata gccagaagtg aaaatgatta atatctctag |
| 51841 | ctattagtgt agcataaatt ccatttttat agcatatata cctatatgga tgatatgtag |
| 51901 | cttaatttca taataatata gagatcattt ctgtttaatg ttgagttgtg tgaggacaac |
| 51961 | acacacttgg gcacacgtgg atgacagaag ttttaccctc aggctactct ggcccaaaga |
| 52021 | gattaagatt ctcctaatct agcattaacc agagatactt tattgttgta gaacacaggt |
| 52081 | attaaaacat tctatcttta ttaataYcct ggaggttagt aaggcaaaat tggccacaga |
| 52141 | aagcaccatt agatttctgc atcagaacag tgtgaataca cagttccttg aacaagccat |
| 52201 | gctattttat gtccctaaga cttcttccat gatctttctc ttttctagaa tgttctatat |
| 52261 | gattcagttt aggtcttact tcttgtacca aatattttaa tcacactggt tattgagtgg |
| 52321 | acacaataat acaatctaat tagatagtat gatagcctgt tgaaagcctc aagtattaca |
| 52381 | ttgattttct attttttaat tagtttccat tctatttgct tctcagattg cctttaccaa |
| 52441 | agattatttt ctctgcaata ttcttatact cattcttctt cactcttgtg ctgaacctta |
| 52501 | cttgttgata tgttatattg tgaatcaatc ctccctatgt ctatgtttta tatcttactg |
| 52561 | ctttcttcat gcgttctggg agaaatcttt atttgatttt ctaaacaagg ttctgtactt |
| 52621 | ctctttgctt gctttcttct tttttgtttt taaaatttgg caccaaggtt ttgtgtttct |
| 52681 | gaaaagtttg gtagggtcct gagttggtag cttaatcttc acctgctgat ttttctgtcc |
| 52741 | tatctagtag ttttctgctt atattttcat ggaaccccta gatcaatcag tatcaccagc |
| 52801 | tgccactttt cattacaggc Rttgtatagg tctcgtatta ttgttacatt tttgtacagg |
| 52861 | gctggtgata atttgaggat agcttggttg gaagtatgac agggtcgggg gaatgtgact |
| 52921 | ggaaatctta aagacaggcc tcttgaccat tgtggataag tggatcctgt gctgtgcaga |
| 52981 | tgaaggtctg gtctggcaat tctgaagggt tggaggtaat attacaggaa gattttctaa |
| 53041 | tcaatggtgt ttgttatgga taactttact ttggcaaagt gaccaaaaaa taaatatttt |
| 53101 | ctctggcata attatggaca tattttgaag gtgaaatata ttatgagtta gcaattcttc |
| 53161 | tctatgtggg aagcagaaga gttctagctg tgctttaggg ttttatgggt gagagagagt |
| 53221 | agttgtttgg gcctacagac ccagctgtgt tactaggatt gctgcctctg atttcaccag |
| 53281 | acactgttta acatgggaag tggagataag tagttctgcc taggtggctt atggtagacc |
| 53341 | Rtcaggtagg aaaggaaggt cagcgagaag gagcaSaggg gcgtgccatg cagacatgat |
| 53401 | tcactgtgtg ggtggctgag gaaactgcag tcctggtgga aggcaggctt ctccttggct |
| 53461 | ggtgagtgca ggaagacaac aggtgatgcc tgcaaatatc actcagctca gacccttacc |
| 53521 | ctgacattct ccagtgtaag caatgagaga aagagattaa tgtatcagga aataataaac |
| 53581 | atcagctatg atctcaccac acacaaccac aacagaggtg acggaggagc tgaaacagac |
| 53641 | actgtcagtg agtgagggtg gggtcaaatc gagaagcagg actgcagcag gagcttaggc |
| 53701 | acttgtaggt gagtggacac tctcagcaat ccacttgcct tttacaacag atgaaaactt |
| 53761 | aacatacccc taatataatt cttgatttct acttcctaaa acctgctctc cttcatacta |
| 53821 | atttgttatg gctaccctaa aaaaatgccc cacaagtggc ttaaacaaca gaaatttact |
| 53881 | ttctcatagt tctggagctt gaagtacaag attaaggtgt caacaggttt ggtgtcttct |
| 53941 | gaggccctgt ctccttcact tctagatggc cggttcttgc tgtatcttca catggggttt |
| 54001 | tatctgtaga cgtgcatccc tggtacccct ctgtggtctg tgctaaYgtt cttttcttat |
| 54061 | gaggacaggt gtcagatggg attagcacca accctatcag cttcatttta acctaatcac |
| 54121 | ctctttaaag gccatgctgc aaatagagtc acattcagtg gtatcggggg taagggattc |
| 54181 | aacatgagag tttggaggga acacaattta gctgataaca cctcacaatc ttttctcagt |
| 54241 | tcatgacatt tgtttcactc aaccattcaa aaacataagt gtcactcttg attgtttagt |
| 54301 | ctcctccgtg tactgccctt agcaaatcca attggttcta ccttcaaaag atatcccata |
| 54361 | tatgatcagt ttcataatct ctatcagaag gYcctgagtc aagccatgat tctccctcct |
| 54421 | ggaggacttt caacagtctc cagctgatct tggtgacatt cctgcccctc accacatccc |
| 54481 | aWgcagttct ccttacaaca gccagagtga tcttttaaaa atagaattta gaacatcagc |
| 54541 | aagatggctg actagagttc cctggcactc atttccccac aaaaagaaat caagaaaaca |
| 54601 | aacaatttta tgtcagccag tgtgatttaa aaagtatgct ggagaccacc aggggaacag |
| 54661 | aaacacctct gtgaagtaca gaaactcaat atagcatcct gcctctgaca tactctctct |
| 54721 | tcagtcaagg ttggctcagg gtcatcagta actccattct acaggaaaaa gtaagctgta |
| 54781 | gttccccagt ggtccacatt gccataaaaa atgtcaacaa tctttgcccc actgttctca |
| 54841 | ctggcacaaa aaccagtctg gagagcagtt gggatttcca aactgcattg ccttacatag |
| 54901 | taagagacca ccttgagcac cctctgcccc tgcaatgtaa ggtgctatgg ctcagtgcca |
| 54961 | tctccaaact gaacctacat agagagtgtg tcttgctttg agcatcagta gcacctggct |
| 55021 | ctctatcact tgaggcccca ccatcattcc aacacattca tacaggtatc tgcagcacca |
| 55081 | tgaccctagc ttcccagagc ctagccagat aaactaatca agaccccggc atccgaaccc |
| 55141 | atgtggtgca ccaccaatcc agggaagaag tgaacagcag ggaagccacc catctgccta |
| 55201 | ctagccacga cacccacatg cacccacacc tcacaaccag ctagtctgct gagcctgtgc |
| 55261 | atgctggtgc ccagcctgaa aactggtcct gtggtgggcc cacccccagg actacagcct |
| 55321 | gcttgggcca caacaactct gagtttgtgc atggtctaat aattggtcct gcagtgaccc |
| 55381 | tgcctcctca gatgggactc tgcagagatg gatggccctg ctacacctgt gagcactgac |
| 55441 | agcaacccgc ccacccagca ggaagacMgc agtgcatgta agtgcacaca ccttgagaaa |
| 55501 | aggctcttcc cactgctgct ggtggcacag ttgctgctgt caccaccggg ggctgcagca |
| 55561 | gtgaaatgcc agttggaccc agcaaagtgg caggatcctc agcattctag catatgcagt |
| 55621 | gttctgcacc tcaggcactg gaaaggctgt gaaccagaca tagggagcca aagcacatgc |
| 55681 | tttccagaac cagagagctg cctccctgtg gctgctgaca cagacagcaa tgtcaccccc |
| 55741 | gcaacacagc agcagagatg ctgcacactt gcatgcaacc tggggacagg ccctctccat |
| 55801 | ctgctgctga gtctgctgat gcagctgggg gccagagcat gtgccactgg cagtgacctg |
| 55861 | acttccacca gcagcagagc cactgtgaac ttgcacgtac cctgaggact ggcttatgct |
| 55921 | gctgccagaa gccaaagtgt gctccacatt gccagagagc tgcctgctag tggctgctgc |
| 55981 | cactgacaat aaccacaccc tgccctagca gcacagctgc agcaaatttg catgtgcctc |
| 56041 | gaggacaggc tttcttcagg tattgctcct gctattggga gatccaagga ttaccccatc |
| 56101 | caacttacca cagccaacat ccatgtacat aactaggggg actgaggaca ggcctgccaa |
| 56161 | gcctggttcc atcatcccag tgcccaggca cactacccag gggtgtggca atcatcctgc |
| 56221 | tttgtccacc accactggca tttgcacatt cctttaggag gactgagaat gggcccaacc |
| 56281 | agcctgacag tagcactgta gccagaaaca atatgcatgc aacatctgaa ggtctggaga |
| 56341 | ctggccccca gcccatcaca accacctcta acaccagagc ttgcaacttg agaatctgag |
| 56401 | ggttgtccaa ccaagctaca gaaattgccc atggcaagca cattcccaga ggcctaaagg |
| 56461 | cccacccacc ctttcagcct accattgcaa ctgctagtac ccaggtaagc tacttaaagc |
| 56521 | ccccagaatt gttctgctta aagctgctaa aactggtaca aacatatgtt gcctaggtgc |
| 56581 | caaatatagg cactctcagc ccactgttac ccctactggg gcccaagaac tggcccacct |
| 56641 | aaaatctctg tccccaggaa aacctcacta cagcctccaa tcacaactac agcctaagcc |
| 56701 | actgaggaag tcacagacac cacttatgct gttaatagct gaaggaataa tataagacca |
| 56761 | ccttactgta cacacacaga atcaaagcca aagtgtccta ctgaatcaac accacagata |
| 56821 | catcttcaga aagaaaagtt aatgttttgg atctatgtca ctgcccaaat ctcatgttga |
| 56881 | attataatct ccaatgttgg gggaggggct tcattggagg taattggatc atgggggtgg |
| 56941 | atccttcatg aatggtttag caccatcccc tgcgtactat tcttatgacR atgagtgagt |
| 57001 | tctcatgaga tttgcttgtt taaaagtgtg tgtcacctcc cccccaccca gtccttctcc |
| 57061 | tgctatgtaa gatgcctgct cctgctttgc catttgccat gagcaaaagc tccctgaggc |
| 57121 | ctccccagaa gcagatgtca ctatgcttcc tgtacaacct atgaaactgt gagccaatta |
| 57181 | aacctctttt cttcataaat tacccactct caggtacttc tttatagcaa tgtgagaatg |
| 57241 | aactgataca gaaaattggt accgaagagt agggcattgc tataaagata ctaaaaatgt |
| 57301 | ggaagcgatt ttggagatgg gtaatgagca gaggttagaa gagtttggag gactcagaag |
| 57361 | acaggaagat aagggaaaat ttggaacttc ctaaaaactt gtaaattgtg accaaaatgc |
| 57421 | taatactgat atggaaaatg aagtctgggc tgaggagatc tcagataaaa ataaggaact |
| 57481 | tattgggaac aactggagca gtcacttttg ttatgcttta gcacagagcc tgggtgcatt |
| 57541 | gttcccctcc tttagggact tgtggaactt tgaacttgtg agtgatgatt tagggtatct |
| 57601 | ggtggaagaa atttctaagc agcaaagtgt tcaaaatatg acctggctgc ttctaacaac |
| 57661 | ctaagctcat atgcctaagc aaagaaatga gctaaaactc aaatttgtat tWaaaaggga |
| 57721 | agcagagtgt aaaagtttgg aaaacttgca gcctggccat gtggtagaaa aaaaattccc |
| 57781 | attttctaga gaagaattta ccctagctgc agaaatttgc atgagtaaag aataaccaaa |
| 57841 | tgttaacagc caagacaatg ggagaaagac attgaaggca tttcagagac tttcgtagcc |
| 57901 | acacccacat cacaggcctg gagtcctagg agggcagaat ggtttcctgg tccaggcgca |
| 57961 | gggccccact gacctgcaca gcctccaaac actgtttcct gcatcccagc tgctccagct |
| 58021 | ccagctgtgg ctcaaagggg tccagctgca acttgggctg ctgcttcaga ggatgccagc |
| 58081 | cataagcttt ggcagcttcc atgtggtgtt aagtctgtga gtgtgcagag tgcaagagtt |
| 58141 | gaggtttggg agcctctctt ttgatttcag aggatctaca gaaaagccta aatgtccagg |
| 58201 | cagaagcctg ctgcagaggc agtgccctca cagagaacct ctactagggc agtgcaaagg |
| 56261 | gaaaatgtgg agttggaacc cctacacaga gtccccactg gggcactgcc tagtggagct |
| 58321 | gtgagaagag agctgtcatc atgcagaccc cagaatgaca gatcctctgg cagcttgcac |
| 58381 | catgcacctg gaaaagctgc aggcactcaa tgtcagccct tgagagaagc tgtgggaaat |
| 58441 | gaaccctgca aagctacagg ggtggaactg aaaaggcctt gggaacccac cccttccatc |
| 58501 | agtatgcctg gttgtgaaac atagaaagtc aaatgaggtt attttgcagc tttaagattt |
| 58561 | aatgacttcc ctgctggttt cagatttgca tggggtctgt agcccctttc tttttacagc |
| 58621 | tttctctttt ttggaacagg agtgtttacc caatgcttgt actccccttg tatcttggaa |
| 58681 | gtaactgttt tttgaattta caggctcata ggtgaaagag actagccttg tctgagatca |
| 58741 | gcctttggac tttcgacttt tgagctaatg ctgaaatgat ttaagacttt gggggactct |
| 58801 | tgggaaggca tgattgtatt ttgaaatgtg agaaggacat gagatttggg agtagtgatg |
| 58861 | ggctgaatga catggtttgg atctgtgtcc ccacccaaat ctcatgttga attgtaatcc |
| 58921 | ccagtgttgg ggaagactgg catgttggga ggtgactgga tcatgagggc agattcttca |
| 58981 | taactggttt agtactattc tctcactgct gttctcatga tggtgagtaa gttctcatga |
| 59041 | gatcttgttg tttaaaagtg tgttgcatct cgcccccact tggtcctgat cctgccatgt |
| 59101 | aagactcctg ctccagcttt gccttttgcc acaagtaaaa gctccttaag gtttccccag |
| 59161 | aagcatatgc ttctatgctt cctgtacagc ctgcggaacc atgagccaat taaacctttt |
| 59221 | tttgaaaaat aaattactca ctctcaggta tttctttata gcaatgtgag aatggactga |
| 59281 | tacaaaaatc ttcccctatg aaagccaatt taaaaaacag aagaagcaac tatttcacca |
| 59341 | gatgcgcaga catcaatgta aggacacagg aaacatgaaa aagcaagaaa atatgacacc |
| 59401 | tccaaatcaa cacaatactc ctccagcagt agattccaat gaaaaagaaa tttatgtaat |
| 59461 | gttggggaaa atatttaaaa aatgatacta atggagctca gtgagataga agagaacaca |
| 59521 | ggtaaaaaat agaaagaaac cagaaaacca tttcaggata ttaaagagaa attcaacaat |
| 59581 | gaaatggata tcataaaaaa gaatcaaaca gaaacgctgg agataaataa atcaataaat |
| 59641 | gaaagaaaaa tacatctgaa agcttcRaaa atagaatagg tcaRtgagaa gaaagaatat |
| 59701 | cagaacttga aaacaaatat tttaaataac ccagtcagac aaaaaaagag ggataaaatg |
| 59761 | tgaaaaagat gaacaaatcc tacatgacat atgggatgcc ataaagcaac caaatttttg |
| 59821 | aatttgggtt tttcccaaag gcaaaatgaa aggcaaaaac ataaaaaacc tatataatga |
| 59881 | aataatagaa gaaaacctct caagtctagc aagagagtta aacacctaga tacaggaagc |
| 59941 | ttagatagat acaacccaaa aatattttct tcatgtcaca ttatactcaa actgtcaaaa |
| 60001 | gtcaaagaga aaaacactat agaagtggca gagcaatatg gtggaataga aggctccacc |
| 60061 | aatcatccca cccacaggaa caccaaattt aacaactgcc tacataatca aagcacttcc |
| 60121 | ataagaacca aaaatcaggt gatcactcaa agtacctgct tttaacttca tattactgaa |
| 60181 | agaggtactg aagagggtag gaaagacagt cttgaattgc catcgccacc cttgtcttaa |
| 60241 | cccagcagca actacataga atggagaatc tgtagacttg gggagggaaa gtgtggtgat |
| 60301 | tttgaaactt cgcattgaaa tcagtgctgc cttgtgacaa tggaaggcaa aaccggggtg |
| 60361 | aactcagctg atgtccaccc acagagggag catatagacc agccctagcc agaagagaac |
| 60421 | tgtacattcc agcagccaga acttgagttc tggcaagcct tgtcactgta ggctaaagta |
| 60481 | gtctgggtct ctaaataaat ttgaaaggca gtctaggcca caaggactac aaatcctagg |
| 60541 | tgagtcctag tgctcagctg ggctcagggc aagtagactg gtgggggcgg tgggtacatg |
| 60601 | acctactgaa acatcagctg gggtggctaa agcagtactt gggccatccc tccctcaacc |
| 60661 | cccagctaca cagcttatgg ctccaaaaga gaccacttcc ttccacatga ggagaggaga |
| 60721 | aggaagagtg aggaggactt tgtctggcat cttggatacc agcttagcct cagtaggata |
| 60781 | gagctctggt cagagtcatg aagccctgat ttcagactct agctcccaga tgacatttct |
| 60841 | agacacacac tgggctggaa ggaaacctac tgcattgaaa ggaagagcca agtaatgaaa |
| 60901 | gaaccaatca cctgctgatt aaagagcact tgggccttga ataacaagcg gtgatatcca |
| 60961 | ggtagtatgc tgtgggcctt gagtgagact ctgagatgtg ctggtttcca gtgagaccca |
| 61021 | acacattcct agctgtggtg ggtatggtca gagattcctc ctgcctgaga aaagcagaga |
| 61081 | gaaaaagtaa aggggacttt gtcttgaacc ttaggtacca ggtcagccac agagaagtat |
| 61141 | aacaccaagt gggctcttga ggtccccaat tccaggccct ggctcttgga cagcatttat |
| 61201 | ggacctgctc tggtccagac ggggagccca ccactataaa aggtgagtcc tagatctggc |
| 61261 | agcattattc accacaggct gaatgaagag cccttggtcc ttaagtgagc atcagtggta |
| 61321 | gcctgagaat attcccatag gcttgtggtg atggtggcca gggggcaagg tgcctgcata |
| 61381 | tgaaaaggga agattagaat gggaagaatt gcatctactg gtttgagtgc cagcttaaca |
| 61441 | gcagtacaat agaacacaag gtaaatttcc aaagttttgg actctggtcc gtggctcctg |
| 61501 | gacagcattt ttggacctgc ctggagctaa gggatcttgc tgctatgaag agaagcacat |
| 61561 | aagcctatcc gtaagcccca aagcagtact aagaaggaag attattgcta tatgtgccta |
| 61621 | catcaaaaaa taaaaaaact tcacataaac aatctaatga tgcatcttaa agaactagaa |
| 61681 | aagcaagagc aaaccaaacc cgaaattagt agaaaaaaga aataaagact agagcataaa |
| 61741 | taaatgaaat taaaattaaa aaatacaaca gaagaatgaa acaaaaagtt tgttttttga |
| 61801 | gaagatgaac aaaattgaca aaccttttga cagactaacc aggaaaacaa gagagaaaac |
| 61861 | ccaaataaat aaaattagag atgaaaaagg agatattaca acctacacca cagaaattca |
| 61921 | ggggattatt agagactact gtgagcaact atatgctaat aaattgaaga aactgataaa |
| 61981 | ttcctagaca aatgtaacct gccaagattg aactatacag aaatccaaaa acctgaatag |
| 62041 | acMaataaca agtgatgaga tcaaagccat aataaaaact gtcctagcaa acaaaagacc |
| 62101 | aggacctgat gacttcaatg ctaaatttta ccaaatatct aaagaagaac taatactgat |
| 62161 | ccttctcaaa ctattctgaa aaatagaaag ggaaggaata ctttcaaact catttggtga |
| 62221 | ggtgaatatt accctaatat gaaaactaga caaagacaca ttaaaaaaaa tacaggccaa |
| 62281 | tattttggat gaacattgat acaaaaattc tcaacaaaat gctagcaaac agaatgcaac |
| 62341 | agcccattaa aaatacaatt catcattact atgtggcatt tatcccatgg atgcaagaat |
| 62401 | aattcaacat gtgcaaacca atgaatgttg ataaatcata taaacagaag gaaggacaaa |
| 62461 | actaatatgc attgcaatag ataatgaaaa tcatttgata aaattcaaca ttccatcatg |
| 62521 | ataaaaatca tcaaaaactg ggcatagaag gaaaatgcat caacattatt aaaaccatat |
| 62581 | atgacagacc cacacctaat atcatactga atggggaaaa actgaaagct ttcctctaag |
| 62641 | atctggaaca tgacaaggat gctcactgtc atcattgtta ttcagcatag tactggaagt |
| 62701 | tctaactaca gcaatcagat gaaagaaaag agaacggaag ggaagggaag ggaagggaag |
| 62761 | ggaaagggag ggaaggggag ggaaggggag gggagggcag gggagggcag gggaggggag |
| 62821 | gggaggggag gggaggggag gggagggaaa agaaaagaga gcatccaaaa tggaaaggaa |
| 62881 | ggagtcaaat tatctttgtt tgcagatgat qaaactttat gtttggaaaa aaataaagac |
| 62941 | tccacaaaaa tgtattagaa ctgatagaaa cattcagtaa aatttcagga tacaaaatca |
| 63001 | acatacaaaa ttcagtaaca tttttatatg cccacaggga acagtctgaa aaagatcaag |
| 63061 | aaagtattct tatttacaat agctacaagt aagataaaat acctaggaat taaccaaaga |
| 63121 | agtgaaagat gaaaagtata aaagtataaa acattggtgc aagaaattga agataacaca |
| 63181 | ctaaaatgga aagacattct atgttcacgg attggaagaa tcagttttgt taaaatgtcc |
| 63241 | atactaccca aagcactctg cagattcaat gcaatcccta tcaaagtatc aaYgacattc |
| 63301 | ttcacagaaa cagaaaatac aattctaaaa ttcatatggt accacaaaag actcagaata |
| 63361 | gccaaagctg tcctaagcaa aaagaacaaa tttggaggaa tcacattacc tgactttaaa |
| 63421 | ttgtcttaaa tactacagaa ctatggtaac caaaatggca tgacactggc atagaaacag |
| 63481 | acatKtttgg actgtgagcc tggcctctat gaaccatcgt ttcagctcct gattggtcca |
| 63541 | gagccaaggc cctgggccaa gctgagtcac aagttctcca agacagccca tggactaagt |
| 63601 | gcattccttc cccttcccag tccataaaca ccctggaccc cagcctcata gagggcaatc |
| 63661 | catttgggtc cctctttccg ctggcagaga gctttcttct tttgtttgtt aaacttatgc |
| 63721 | tttaacctca cattgtgtct gtgcttctca atcatcttgg acgtaggaca aagaactYca |
| 63781 | gatattatct cagacaataa gagactgcta tctgggtccg ttggcaaaac tacaacatta |
| 63841 | caacaagaaa actttgggga aactctccag aacaccatag tgggcaaaga tttcttgagt |
| 63901 | aatacctcac aagcactggc aactaaagca aaattaaaat taaaaaaaaa cttcaaagat |
| 63961 | aaaaattatt aaaaagagaa aagcatccag tcatatgtaa gggaactatc accagatcaa |
| 64021 | aagagaattt atcaacagaa agtttatagg ctaggagaga atagtatgga atattcaaag |
| 64081 | ttctggaaga aacactttca gtcatacata ctatactcag aaatgttatc cttcataaat |
| 64141 | aaagaggaaa taaaatcttt cacagataag caaaagcttt agactaacct tacaacaagt |
| 64201 | gctcaaggga gttctacatc tRgaagcaaa cagatgataa tcactatttt gaaaatacat |
| 64261 | aaatactcac tgttagagaa gacacacaaa gaataaagag aatcaagcct tattatttca |
| 64321 | gaaaactaaa agaaatgata aacaataaga gaggaagaaa ggaagaaagg gacatataaa |
| 64381 | acaaccagga aacaatgagc aatatagaaa aaccaaaacc tcacatgtga ataataaccc |
| 64441 | tgaggtaaac aaattaaatt ccccatttaa aagatatata agtcatataa aagggaacct |
| 64501 | ccattagatt aacaacgaat ttgtcagaag aaaccttaca ggccaggaga gaatgagatg |
| 64561 | acatattcaa agtattgaaa caaaaaaaat cttatcagcc aagaatacta tttccagcaa |
| 64621 | agctattctt caaaaatgaa gtagaaataa tggcgttcct aaacaagaaa aactggaaat |
| 64681 | tcatcactac tacactggtc ctacaagaaa tcctgaagga aattctatac tttaaagcaa |
| 64741 | aaagacaata tggtccatca caaaaacaca cacaagtata aaactcactg gtaaaccaaa |
| 64801 | tacacaaatg aaaaagagaa aggattcaaa ttttaccact acataaaatc aacaaatgac |
| 64861 | aattataaac aataaaaaat atagaaaggt aaaaggacat atgaaataac cagaaaacaa |
| 64921 | ttaataaaat gacaagaata aatcctccgg tagtaataag cttgatggta aatgaactaa |
| 64981 | aattttcagt ttaaagatat agactggctt aatggatttt aaaaagcgtg acacaagtat |
| 65041 | atgctgccaa taataaagta aatttctctg taaagacaca tggacttaaa gtgaagggat |
| 65101 | gaaaaatgat attctatgca aatagaaaca aaaagtgaga agtactatac ttgtatcaga |
| 65161 | taaaacagat tttaattttt aatttgcaaa cagcaaaaag acacaaaaaa ggtcatcatg |
| 65221 | taagataaag ggatcaattc agcaagaggg ataatacaat tctaaatatt caaacaacac |
| 65281 | tggagcaccc agatatataa ggcaaatatt attagacata aagggggaga tagattccaa |
| 65341 | cagaataata gtgagggact tcaatgcccc attctcagcc ttagacacat aatccagagt |
| 65401 | gaaaattaag aaataaacat tggattttaa ctgcacttta gacatgtctc aaaaaaaagt |
| 65461 | atacgaatgt ccagtaggta tataaaaaat gctcaacatc actcatcatc agggaaatgc |
| 65521 | aaattaaaaa cacaatatca tccttctcca gttagaatgg gtattattaa aaagaaaaaa |
| 65581 | aaaacagatg ctggtgagga ttcaaggaaa aactcttaca taccgttggt gggaatgtaa |
| 65641 | attagtacag tcactatgga aacaatatgg agatttctca aaataactaa aaatggaatg |
| 65701 | accatatgat ccagcaattt cRcttctggc tatttatcca aagaaacaga aatgaggaca |
| 65761 | tcaaagggat acctatattc aaatgttgac tgcagcacta ttcaccatag caaagatatg |
| 65821 | gaatcaacct aagtgtccgt cagtggacaa atggattaaa aaaaaacagt ggtatatata |
| 65881 | cacaatgtaa tactattcag ccataaaaaa agaataaaat catgtaattt gtagcaacat |
| 65941 | ggatggaact ggagggcatt atgttaaggg aaataagcca ggcacagaaa aacaaacagt |
| 66001 | gcatgttctc acttatgttg atcacacaga ggtagagaat agaataatgg ctacaagaga |
| 66061 | tggaccagtg tttgtgaggc aggaggacaa gtatgaagag aggttcatta atgggttcaa |
| 66121 | acatacagtt agaaggaata aattcaaata tttgataaca gattagggtg actatgttta |
| 66181 | acaatatttc aaaatagcta gaagaaagga cttgaacagt tccccaaaca tagaaatgat |
| 66241 | aaatactcaa gatgacagat accctaactt gatcattata cattctatat gcttataaca |
| 66301 | aaatatcatg tgtcYcataa atatgtacaa acattatgta tcaattttta aaaatcagga |
| 66361 | ttgatagctt tactgctgaa ttctaccaga catttaaaga ggaattgata ctaattatcc |
| 66421 | tgaaacaatt ccagatattg cagaggaagt aatacttcca aacgcatttt acaaagccag |
| 66481 | cattaccctg attccaaaac caaagataca acaaataaaa aagccacaag ccaacatccc |
| 66541 | tgatgaatat caatgcaaaa attcaacaag attttagcaa agtgaacgca gcagcacatg |
| 66601 | aaaacgatca ttcactatga tcaagtgggt tttattcaac atatacaaat caataaatgt |
| 66661 | gatgcaccct attaacaaaa tgaaggacaa aggccatatg atcatctcta tagacacaga |
| 66721 | gaaagtattt tataaaattc aatatccctg cataagaaaa gctcacaaga aattagacac |
| 66781 | acaagatatg tacctcaaca caaaaaggcc atatatgaca aacccacaRg taacatcatg |
| 66841 | cagaataagg aaaggtgaaa gatttcctct aagattagga taaagataag gatgttcact |
| 66901 | ttcatcactc ctattcaaca tagtattaaa agtattagct agatatacta ggcaagagat |
| 66961 | agaaaYtaaa Maaattgaaa ttgaatagaa ggaagtcaat tgttcatctt ttYggatgac |
| 67021 | atgatcttat ttatagaaaa ctctaaagac ttcaacaaaa aactgttaga ataaatctgg |
| 67081 | taaagttgca gaatagaaaa tcaatgtaca aaaattagta gcatttctgt atgctaatgg |
| 67141 | tgaactatat gaaaaataat tcagaaaaca atttcattta caataaccaa aaaagaataa |
| 67201 | aatacttagg aataaactta accaaggaga taaaagatct ctaatactgc aaactatgaa |
| 67261 | acattgataa aagaaattga aaaagatgca aataaatgaa aagatactct atgtttatgg |
| 67321 | attggaacaa ttaatattgt ggaagtggcc atactaatca aaatgatctt cagatttaat |
| 67381 | gcaatcccaa acaaaatacc aatcagcata cttcacagaa ataaaaaaag cacacacata |
| 67441 | ttttccgtct ttgagtaatg tatgctctga aaggggaaaa aggcattgag atacaaaagc |
| 67501 | acattataag atagaaagct ctatgtgcca tttcctgtca tttaccttga tattagatcc |
| 67561 | aggaaactct ctctattttt aggccaaatt attagcaaac ttaattgccc ttcgccatgt |
| 67621 | aatttaacat aaaacacagg ttctgggaat taagatgtaa acatctttgt gaggcctata |
| 67681 | ttttcctcct gcagagagaa caggtaatct tacatctctc aaatttgttt cagttgtgta |
| 67741 | tggagacagc tatccctgat ttggtaatgt ttaaactaga cttttgcaaa aggatatgta |
| 67801 | tgagtttata taaaatttga tagtattgca tgtatttata ctgtgcaata tgtatatgca |
| 67861 | atagtttaag tgacaaaact aagtggtaca aatttacctt gaggttggct gcatttgtaa |
| 67921 | gttggggcag tcacttcttt ggagtgttgt gcagacttgg gaatgagaag ctgtagattt |
| 67981 | cattctaact ctgccactta cagcaggaca accacttgca aagcccagct ttctctatta |
| 68041 | aaataagacc ctaaatcttc ccaacaccaa taatgaagat gacattctca gatcaggaaa |
| 68101 | atgaagggtt cttcagaagg atcatcactg tgtcaggggc agcaactgga gatctgaaca |
| 68161 | aaatagctac ctgggcatcc aatccctcta ctgtgaaatg catgattctc cagacctttt |
| 68221 | ccaacacgca tagagctttc tgatcgtttt agtgcctact cttaagtatg aattgaatca |
| 68281 | aaggttgacc agacaattga agaaattctc catcaaatga gactcaaaga atcagaacaa |
| 68341 | agtgtaattc tgaaggaaaa gaggtaagat aaacagcaga agaaacgttt acaagactat |
| 68401 | aattaatata ttctgatata accataaaac ataaaaggat gctatcaaat agaagattga |
| 68461 | ccagagaaca caaaagaata tgagcaattg taaattaaat tgataagttc aataaaatgt |
| 68521 | aaagttaagg aaatccccta tgaggtaggg caaaaaggca aagatatggt ccataatgtg |
| 68581 | aagacaatta aaaaattcac ccaacatgaa ttttagaaaa agaaaacaga gtatatattg |
| 68641 | ggaaagacat tatcaattaa atactgccat acattaccca gaacttatgg ataattctct |
| 68701 | ccataatgaa aaaagccatt gattatctaa aagaccagat ttaacatttt caggccccag |
| 68761 | tgcaggaaga caaatggagg ctcacatact ctatctctga ataaccatac attataaagt |
| 68821 | aaaatcgacc ctaggttcag gttaggtttc catccaagtc ctgaagcctg tcccttggag |
| 68881 | actgcccttc taacctggcc tagagacaga agaatcatgt gtttccctgc accaacactg |
| 68941 | actgatcctg caagctgaga ggtgggaaaa tacggagtgg cagggctgtc tgctggagca |
| 69001 | gtgggcagga gctgagatga ggcccagcac tgacctggcc aagagacaga agaatcatgt |
| 69061 | gtttccctgc accaaccctg actgatcctg caagctgaga ggtgggaaag tatggagagc |
| 69121 | ctctaatgag ttttcagttc agcaaatcta gttaccagtc cccagatttc tgtttgattt |
| 69181 | ttcaaaatta tttcaatctc tgttaaaatt ctctgataaa tcaataaatt gctttcctat |
| 69241 | gctatcttgc agatatctga tttccttaaa attgttattt tgaattcttg atcagagagc |
| 69301 | taaccaactg tcatgtcatt ggggtcagta actagtttgg tacctttgtc cttttgggaa |
| 69361 | ggccatggct ctttgcttgc tgttgtttcc tgtgggtata cttctatgtc tttccataga |
| 69421 | agggttatta attaattcca gtttttcagg tctgggttgt gtttgttgtt attgagtcta |
| 69481 | tttgcttggc aaatcctcac tgctgggctg ttgcctccct tttgactcta ggtggtgtct |
| 69541 | caacctcact tttaccttgt ctttagtaaa cggttggagt attaactgtc cgaaatggga |
| 69601 | aatgtcccaa aggaattacc ctggaagtat gagaaacttg gctaggggtt atggtctggt |
| 69661 | gacctaagga atgtacctcc taaagcatgg tgctattgaa catctaccct gatttggctt |
| 69721 | ctcctttggc caagttatga acagtgtcca gaactggaga tgatcttccc tcctccctat |
| 69781 | tttgtctctt gctctcttca ggaatattcc tccctgagga cagagaatat tcatcttccc |
| 69841 | taagggagtc ataatgcatc tcatgggcta aggaagaaca agtttcttgc cagggaatcc |
| 69901 | aaggtggtaa ataatctgtt tgagaacatc aatttcactt tattcactct aaaatccatg |
| 69961 | aattggggga gattctctgc atgtgtggtg ccaggaagaa taggaaggag ggacatcatg |
| 70021 | gaggtggaag tcccatctcc tactgtcttc ttgatgattt tccacttctc tttggccctg |
| 70081 | agaaccatct cattttcata cttgagctat gtatttttcc tgacatagat ctcaaagctc |
| 70141 | tatatttgtt tttggtattc tgtttgttgg ggtagtaagt cagcttgcct ctacagcaac |
| 70201 | actttggaac tagtgggtag ctattttgtt gagacaacaa tatgctatct ctgaaaccac |
| 70261 | actgaccctt cagaaaattc ctccctcttc ctgacctgca agaataataa tcactgccga |
| 70321 | cactttggag ctggtctggg ttcatcccat tccatccagc actgctggca cccaWgtact |
| 70381 | tctctgatgg gtctgaggac aaacctacac agcctgtgcc aagaccattg ccagtacgta |
| 70441 | cctgcatgtg acaccttgga ttcttaggac tggcctacat aggccattgc catcaccatt |
| 70501 | accaccagca cattcaacct ggaagcctga gggttgtctt gctactgcta ttgctatcac |
| 70561 | ccatgccagg cacattgcac aaagtctcaa gaaccactca ctcaccaggc ccaccactgc |
| 70621 | aacttccagc accacaggaa actccctgga ggtacaataa tttacccaac tgaaactgtg |
| 70681 | gatattggtg tcaatgtttg ttgtctggag gctcaaagac cggcatgctc attccactgc |
| 70741 | tgccactaac atgtgttgca gactggccca tctagtaccc ctgttcccat caaaacctca |
| 70801 | caaaaacttt tactaacaac ctcagtctta gcaactgagg aaatcacaga ccactgatcc |
| 70861 | cgactagaga caaagaaacc atacagatat tatgctagtg tatacaccca gaatcaaagc |
| 70921 | taaagtgctg tatgcaacca acactacaga tatatctata gaaaaaatag tgttccccta |
| 70981 | tgaaagccaa tcaagaagag gctgttactc cagacgtgca tatactaata tagggactca |
| 71041 | ataaacatga agatgctaga aaacatgaca cccctacagg accacaatta ttcttcagca |
| 71101 | ggagattgca acaaaaataa aatctatgaa cttctaaaaa attcataaat atgatatcat |
| 71161 | agaagcttag ttaaatataa tagagcaaag ataatcaata caaaaatctg aaaaaaatca |
| 71221 | gaatatgaat aagaaattca ccagatacct agatgtcatg agaaagacca aacagatatc |
| 71281 | ttggtagtaa agaattcaat gaataaagta aaaaaaaaaa aatattttca aaagcttcaa |
| 71341 | cgatagacta ggtaaggcag aagaaaaaaa ttcaccactt gaggactaga tttctgaaat |
| 71401 | aaaccagata gaaaatataa taaatcatct aaataaagcc agtattcaat ataacatacc |
| 71461 | atgtagttac caaaggtttg attctgagta ttccagaata ctcaagaaga agagatgggc |
| 71521 | agaggcatga caaaaatttt aaactaagca catgtaccct aaaacttaaa gtataataat |
| 71581 | aataaaataa aataaaaaaa gaaaaaaaaa taatagctga aaatttgtca agtcaagtct |
| 71641 | agcaagagat ttagacattc aaatagagga cctcaatgat actcaaattg acacaacccc |
| 71701 | agaagtcctt ctccaaggca tacagtggtc aaactgtcaa aagccaaaag tcgttaattc |
| 71761 | taaaaacagt aagataaaag catcgagtca tgataatgga accctcatca gattaacagt |
| 71821 | gattttcact gtataaactg tgaaagagag ataaaatcat atatttaaag tgttcaaaga |
| 71881 | aaaaaaatct gccagccaag agcactatct tcagcacagg cgaacttcaa aatgaagtaa |
| 71941 | caataaagtt tttccctgaa aagcaaaaac agagaattca tcaccacaat gactgcccca |
| 72001 | caagatatgc ttaagggaat tctgctcctg aaagcaaaag tacaatatct accgtcatga |
| 72061 | aaacacaaaa aatttaaaat actccctagt acagcagaca aatgaagact tgaaatgatt |
| 72121 | taaattttac tattatagaa aaatactaaa ccacaataat aaaaagtgag agaaaaagaa |
| 72181 | acaaagtata tgtaaaacaa acaaaaacaa ataacaaaat gaagtaattt ctcacatatg |
| 72241 | aataataatc ttgattgaaa attgattaaa ttctccactt gaaagacata gactggctga |
| 72301 | gtgaattttt aaaaaatatc ctaactatat gctgcttaca agaaactgac ttcacctaat |
| 72361 | aagacacata tagactgaaa atgaagggat agaagaaaat atcccatgta aacagaaaca |
| 72421 | aaaacaagtc gaaataggta tattaacatc agataaaata gactctatgt caaaaacagt |
| 72481 | aatgagagaa aaagaaggtt attatttaag aatagaaaga tcaatttagc atgaggatat |
| 72541 | aacaaaacta aatatattcc cccaacacta gagtatacct agatacataa agctatcatt |
| 72601 | atgacatcta aaaggataaa tagattccaa tacaatagta gtgtaaactt taacacctga |
| 72661 | ctctcagcat taaacagatt atttagacag aaaatctaca aagaaatatt ggacttaatc |
| 72721 | ttaagaataa ggggacccaa tatacattat agacattttt atttaacagt ggcaaaacat |
| 72781 | accatttttc ttattagcct gtggaagatt ttctaataca gaccgtatgt taggccacaa |
| 72841 | aacaagtctc agtacatttt gaaaaagcag tatactttat tttcatatac cacaatggat |
| 72901 | aaaactaaaa atcaatatta agaggaactt tggaaattgt aagaatacat ggaaattaaa |
| 72961 | caacatgctc ctgagcaacc actggttcaa tgaagaaatt tagaagaaaa ttaaaatgtt |
| 73021 | tttgaaatat gacaatgtaa acacaacata cagcacaaat agtgctaaga ggggagatgc |
| 73081 | tctgaataaa ttctttcatc caaaagtaga gagatttcaa ttaaacaacc cagtgatcca |
| 73141 | cctgagggaa ctagaaaact aaaaacaaac tgaaccccaa attagtgaaa ggaaaaatat |
| 73201 | agtaaaaata agagcagaac caaacaacta gaaactaaaa aaaaacaaac ccaaactcaa |
| 73261 | tgtaatgaaa agttggttta ttgaaacaat aaataaaatt cctaagtgtc tagctaggct |
| 73321 | atactatata tgtgtgtgta tatatatata tacacacaca cacacgtata tatatatatg |
| 73381 | ctatactata tatatagtat agatattgtg tatctataca tatataaaaa tatatatata |
| 73441 | gacagaaaaa actgaatgaa acaaaatcag aaactaaaaa gaagatatta caacttatat |
| 73501 | catagaaata caactgtata ttttggacaa taatacacta atgaaaccgt atgttttgaa |
| 73561 | caagtataca ctataattga acaccatgga aaaagattaa ttccctgaca tatacaactc |
| 73621 | acacaaaatg agccaggaaa gaagggtaaa cctgaacaga ctaataatga gtaaagaaat |
| 73681 | agaatcagta ataaaaacga aatctcccaa caaagcaaag tccaggccta gatggtttca |
| 73741 | ctgatgaatt ctaccaaact tataaggaaa aaggaaaagc attcctcatc aaacaattcc |
| 73801 | aacaaattca gagaagagaa tacttcctaa ctaattttac aatgccagga ataccccaat |
| 73861 | gctaaaaaca tacacgaaca caacaacaat aactataaca aaaattagag gtcagtattt |
| 73921 | ctgatgaaca cagacacaaa aatcctcaag aaaatgttag caaaccaaat ctaagaacac |
| 73981 | atcagaaaga taattagcaa taatcaagtg gtatttatcc cagggatgca agaatggttc |
| 74041 | agcatacatg aatcgataaa tgtggtacat cacattaaca gaataaagac aaaaccaaat |
| 74101 | aagcatatga atagatgWag gaaaaagcat ttgataaaag ttagcatcca tttatgataa |
| 74161 | aagttcccaa cgaactacac aaagaaggaa tatacctcta tataatgtaa ccacatgtga |
| 74221 | cgaactcaca cctaacatac agaatgggga aaactgaaag ccatttctct aaaaactgaa |
| 74281 | ataagacacc actcagtcaa cattttactt tatgtcatgt cctagcgaga agaatcaggc |
| 74341 | aagcaaaaaa aataaaagcc atccaaattg gaaaagagga agttaaattg ttcctgtttg |
| 74401 | cagacgacat agtcttatat taggaaaaac acactggggc ctttcagagg gtagagggcg |
| 74461 | gaaggagaaa gaggatcaag aaaaataact aatggtacta ggcttgatac ctgggtgatg |
| 74521 | aaataatctg tacgacaaac ccccatgaca caagtttacc tatgtgacaa acatacagtt |
| 74581 | gtacgtctga acttagaata aaagttaaaa aaattgttaa cataaaaaaa gaaaaatcta |
| 74641 | aactccacca aaaaaattta ggactatcca ataacattgc aggataaaat atcaatatgc |
| 74701 | aaaaattagt agcatttcta tatactagca ataacccagc aggaatgaaa aatcagctat |
| 74761 | gaaattccct ttacaaagct ataaaaaatt aattactcag taatacattt aagcaagcag |
| 74821 | actgatgttt accagctata tgactgaaaa tgtcaggcaa tcatattgag cttttgtttt |
| 74881 | ttcatctgaa gagtaaacat aatgcaagga attattaaag ggctaccata ggaataaaag |
| 74941 | agctaatggt ataaaacatt attagtatta ttagtattgc atagtatcta tgcaatagtg |
| 75001 | taagtcagaa actgaaatgg cagaaatcta cttttaataa ccatttacat ttaatgtaat |
| 75061 | tattggtatt tttgagccat ttttgttttt catatttata ctacttttga ttgtttttct |
| 75121 | ttttcctgat ctatggattc cttgaaaatg ttttttgatt ccattttagt tgatctgtaa |
| 75181 | attttttgcg tgactctctg tacagcattg ttagtggttg ctcYgtgtta cattaYgcat |
| 75241 | acataScttt tcacagccta gtgctgtgca cattttaccc tattcagtaa aatatggaaa |
| 75301 | ccttgctttc ctgtcttttt aactgctccc cctttcccac tgtttgtaac agaatcatat |
| 75361 | taaatatatg tctatgtgca tttagtgcca cagtaaattt tgtcattttt gctttgacat |
| 75421 | aaatttattg tcttccaggt ttgcagctca gaaatctgaa ctgggtctca ctgcgttaaa |
| 75481 | tccatgtctc aaggctgagt tccttcctgg agtctctaga agaaaagctc attatcttgc |
| 75541 | cacattcctt ggccctcttc catctttaaa gctagcaatg gccagttgag ccatttgaaa |
| 75601 | ctcatgcaga aaacgtgaga agaaagatat tttgtgttta cctactttgt atttccaatt |
| 75661 | tttgtttttg gcttgttgtc attgttgttg ttgttgctca ttcttgttgt tgttgctcat |
| 75721 | tcttgatgtt ccagaaaaat cctattctat tttttccttt atgattagtg accttcatag |
| 75781 | aaccatttta taagataggt ctgttggaaa gMaattctct tagatttttt tttctgtctt |
| 75841 | ctaagaatgc ctgtattttc tctttattga tgagtagtta tattggacat aaacatctgg |
| 75901 | ttgacatttt ttttctttca gcactttgaa tattttagct acatcctatt ggttattctg |
| 75961 | ctctctggta ataacaccac tgttactcac attgtgtttt ccctatagct atggtgtcat |
| 76021 | ttgtttcttg ctgctttcaa aacatttttt ttagttttta gaaatttgac tacagtgttg |
| 76081 | cttgcatgga ttttcttggg tatattctgg ttggcagttg ctcatcttct tgcatctgta |
| 76141 | ggtttatgcc cattgcaagt ttggagaatt ttcagccatt atgtctttga gttctctctc |
| 76201 | aatagcatcc tcattgttct tcccttctga agctcagatt ttatcattat cacattattt |
| 76261 | gttataacct ttcttgttca taacagtatt taaaatttta ccctgtgttc gagtatacag |
| 76321 | tttctattaa tccagcctca agaatactga ctctttcctc catcatttcc tttctgttag |
| 76381 | tgatcacacc aaataatatt ttatttaatt ttttacacta catatctcag tatcaaaatt |
| 76441 | tctatgtcat tatattatat agtttctttt ttctgctggg aatttttttt ccatttaaat |
| 76501 | tgagagtgta cacattgacc tcataatgga tgtttgtaac acctgcttca gtgagattga |
| 76561 | aaatctcacc gttttaatat ccagattgtt tcaaggttat catctcttga ttatttaact |
| 76621 | ccttgagaat tggtcgattg tttctgattt tataaagtat gttgagtaat tttctatttt |
| 76681 | atcccacaca caggactttc tctgtttaca gtacacaata agttcaatga aggaagtttt |
| 76741 | ggacattgga gggtagaggc cttcttgctt aattgagatc cactcactgg ttcagatagc |
| 76801 | aggggatttt gtcccactaa catttttcta cttggagctt ctgcctggat gatcctttat |
| 76861 | ggctcttacg agataaaact ataagtatat ttaacatcat agcatacata gtgtgcactg |
| 76921 | aaggggtccc aaaaaattcc tattcacaaa gtctttttta gttttctttt gtgcatcatt |
| 76981 | tgctcaaatc tcaattgaat ttgaggactt cattttcctt tcttccctac agtaacaaca |
| 77041 | cagtttaaca cctgcttaat tagagtctat tacagtaccc gggtaggcct gctgtcttct |
| 77101 | gactggggat ttatagaaag tggataatac ggctgaggta aaatcaacga ctacaggcct |
| 77161 | ggcgaaatgg ctcatgcctg taatccaact actttcaagg gcagaagtgg gagaattcct |
| 77221 | tgacacaagg aatttgagat atgcttgggc aacatagtaa gacccagtct ttataaaaaa |
| 77281 | attacaaaag gatcctggct cggtggtttg cacctgtaga ctcagctatt tgggagggtg |
| 77341 | agctgggagt atagattgag cctgggagag caaggctact gtgagccatg attacgccac |
| 77401 | tgcactccag cctaagagag agagcaagtc aaagtctgaa aaaacaacaa tgaaaaccac |
| 77461 | ccaaaaaaca aaaaagacta gggccaccaa aaaggctttc tttttcactt gacttcttgc |
| 77521 | tccagggttt acctttttaa agtgcacctc ccagtgtcta gggagtctac agacttggag |
| 77581 | tctcttcctt tcttgcctcg gtatcacaag tggctccatc ctatatcctt cagcactcct |
| 77641 | tccccatttc ccacagagcc aagtagaata gtgatttgaa cacatataag caattttaat |
| 77701 | ccctaccctc cttgatgctt tacaaaattt tgggatggat ctgttaagct tttgtctcat |
| 77761 | ttgagtcagg gatatctcag gcccagctca aatccttaaa ggaactgaga tcagagtaga |
| 77821 | ggaatagaag tgataaggga gatatgtata aagtgtcttc atgcaagtaa acaagacaca |
| 77881 | tttaatttca aattcaagca gtgcagacat tgctcaaaac caattgaata aacatttgaa |
| 77941 | ttttaggtct caccaaaggt ctacaacagt gatggagtat ctttaattct tctacctctt |
| 78001 | gttctctatg gaaaaaagca aatgtaaagg tgtcatttgg taagtttgtt tcaattctac |
| 78061 | catcagatac tcagttttca accatttccc tatcctactc attaatgaac agcataaggg |
| 78121 | aaattttcat acattttggt caacatacca gctattggtg tgtccttttt acattaactg |
| 78181 | aagaataata aatagtcttt acagatgtta cgagattaca aaacaaaaag catccaaagg |
| 78241 | atctaaatta gggtgtaatg tggatgccta atggttttct atcagaactc tcaccaattg |
| 78301 | ccctgtctga tgagagaaat gagcaggagc tctgtcgtgg tgaaggactc tgctgaagct |
| 78361 | cccccaggca tttctctact agagttctgg ctttctcaaa atattctcag aataagcata |
| 78421 | tattgtcctt ctctggccct tcagaaagtc aataagcaaa atgccttgag cttcccaata |
| 78481 | aactcttaca attacctttg cactttcctc atccactctt ctttgactgg accacttcca |
| 78541 | cctcttggta gccattgctt tgattgtgct ttgtttcatg attgtactga taaagccatg |
| 78601 | ttatatctgc tgtcacaatt ctttgatgaa atgcttcagg atctttatcc ccttatttaa |
| 78661 | attttcaggg aagcttcttc tcttgtctgt agctgctctg gttgccatag ttttggcatc |
| 78721 | cacagagtga aaaatttgct caacattaat ttttcagtta gaatcatgta agatgaacca |
| 78781 | attaaaaaac atgaagggtt ggctattgtt tgtactctta atcttcattc ctcttctatc |
| 78841 | atggtatcaa caagatacat ttgatatgaa tactctgtca ccgtagactt gaggttcagc |
| 78901 | accatttcat cccttcttga aataacatat ctacttgtga actgctgatt tcttcagcca |
| 78961 | ttatccaYat gaacttttca gaaagcatca gtggtttYac cttctttcat gcaagcttaa |
| 79021 | ccataaattt tctctttgtt ttcactttaa ttttagaaaa atgtattttg ctcttatagg |
| 79081 | ggatgttttg aaactcctgt cttatttttc attattcctt gaaatagctc ctattcagac |
| 79141 | atcttacagc atatcagttt aggttcattt tgatgccaaa aaatgtggaa tccttttatg |
| 79201 | gtatttctta atataacata cattttcatg aactttttaa agacctacta cattacagga |
| 79261 | aggcctttca ataatataag aattgctaca ggtaacattg aattttctag ttgattaggc |
| 79321 | agattaaMtt tctgtttttc tgctgacata aatagtgatg tgggttgaga aggaatataa |
| 79381 | taccagtggt caaatattct gcagctatgt ggaaagtcaa acagttgcaa ctaagtgtca |
| 79441 | catgctttaa gggagtcata gagtagctgc ttgggcctag caggtaccct gatgttaaca |
| 79501 | ggatgattgt ctctggtttc accaaacatt acttcaaaag agttactgga catgagcagt |
| 79561 | tttgcctagg tagcttacag tagaccttca agtaggtaag gaaggtcaga gagaatgagc |
| 79621 | agaggtgaat gccagtcaga tgtgcctctt tgagtgggta gctgaggaag ttccagtcct |
| 79681 | ggtgggaggc aggcttctcc ttgtctcctg agtccaggaa gagaacaggt gatgcttcca |
| 79741 | tatatcactc aactcacacc ctattcctga ccctctctaa tgtcagaaat gagtgagaga |
| 79801 | gatttgtgtt tcaggcaaga ataaatattg ggtatcaacc taccagacaa ccaaaacaga |
| 79861 | gattaccaaa gagctgaagg agacactgtg actgacttag aatggagtca aagccaggag |
| 79921 | caggactgca gcaggcgctt aggcatctgt acaggagtca aggacctcag caatccacct |
| 79981 | gtcatttaaa atgaatgtaa acataccgtg taccaagtag aatcttgatt tccaccttct |
| 80041 | ataacctgtt ctctttaata tcaacaatca ttaatttcag accaagaaac cagaatatat |
| 80101 | tgagaaacac atcaatgaaa caatacattt cccagaacta aaggttaatt ctccatatcg |
| 80161 | aaagggccca atgattatcc aaaagcccaa atttaacatt ttcagggctc agtgcaagaa |
| 80221 | gacaattagt ggctaacata ctccaactct taataatcac acattataaa gcaagctaga |
| 80281 | ctctaggttt agatcacatt tccatgtaag tccttcagcc tgttgcttgg agattgctca |
| 80341 | tataagctgg catagagact aaaggtgtct gtgtttccct gtaacaatcc tgaccccgca |
| 80401 | atctgagagg tgggaggagg ggggcacagg gctgtctgct agagaagcac actagacgtt |
| 80461 | gggctggagc ccaggactga tctgcccaag cttcgagagc ccctgccagg catgttatgc |
| 80521 | tcccctatgc ctcacttctg agtttggact agtggcaatg tctacacagc tcccaatgat |
| 80581 | aaccctactg agcataggac tggcagagaa taataaccag aatatacaaa caaacaactc |
| 80641 | aaaaacaaca aaatagtcca ttaaaaagtg aacaagagat agaaatggac atcttttcaa |
| 80701 | ataagacata caaataatca acagtatgtg aaaaatactc tatatcacta atcatcagag |
| 80761 | aaataaaaat caaaaccaca ctatcacctt accctattca gaatggctat gattaaaaca |
| 80821 | aacaaacaaa caaaacagat gttgggatgg atgtggagaa aggagagctc ttatacaccc |
| 80881 | tgattggaaa gtaaatagta taaactctat ggaaaacagt aYgatttctc aaagaactaa |
| 80941 | aactcgatta ctattcaatc tagcaatctt actactgtat ctatgcagag gaaatgaaat |
| 81001 | cagtatatga aaaataaatc tgcacttcta tgtttattga aacaccatac acaaYagcaa |
| 81061 | agatatggca tcaacctaag taaccatcaa tggatactaa tgggtttgta ggtaaagaaa |
| 81121 | tcgtgatata tacacaatgc aatactactc tgcaaaaaaa aaaaaaaaaa aaaaaaagaa |
| 81181 | ggaacgaaat cactcctttt gtagcaatac gaatggaact ggatgtgaca gataatatgc |
| 81241 | tatgtgacaa aggccagata cagaaagtca agtataacat gttgtcacta ataagtaggt |
| 81301 | gctaaaatgt gtttacatag atatagaggg gagaactata gacactgaag ccttggaaga |
| 81361 | gtgagtgggt gggagggaat taagagttag acattaatta gtaaatgcaa tRtttgttat |
| 81421 | tcttgttatg gaaaccctaa ggccatgatt taaccattat gcaatgtata catgtaacaa |
| 81481 | aattatcttt gaaccacata tatttataaa aataaagttt aaaaataaat tgagggtgac |
| 81541 | cgggtgcggt ggctcacgcc tgtaatcaca gcactttggg atgccgaggc ggatggaaca |
| 81601 | cgaggtcagg agttcgatac cagcctgacc aacatggtga aaccccgtct taactaaaaa |
| 81661 | tacaaaaatt agccaggcat ggtggtgcat gcttgtaatc ccagctactc aggaggctga |
| 81721 | ggcaggagaa tcgcttgaac ctgggaggcg gaggttgcag tgagccgata tcttgccact |
| 81781 | gcagRccagc ctgggtgaca gaatgagact ccatctcaaa ataaataaat aaataaataa |
| 81841 | ataaataaat aaataaatat aattgaaggc aaatttaagc atattttttc aaaaaagaaa |
| 81901 | aagagggaga tggagtagga gaaataaaaa agaaggaaga ggaggaggag gaggaagaaa |
| 81961 | gagcctgcat ttctaattgt cataattaaa tttccataag cagatactat tttttttaaa |
| 82021 | aaaaacctaa ttagaatttt acgggtctga taagaaagaa aatgctaaaa aaaatatgRa |
| 82081 | tactcaRtgt gttcctattt tgtttgaaag cctagaaaaa aacaatatca gagactattc |
| 82141 | tacttagatt ttttggacaa agaaatctta agatttttaa ctcgggaaca tttaattcct |
| 82201 | cacaatttca tgtctgctgt tctttcctaa aatcattatt gtgaagcaga catgactata |
| 82261 | tccactagtg tttcatgtat gtgatctgct tagtcttcat cctttagggg agccattttg |
| 82321 | attattcaaa aatttagaag aggaaactta ggctctgaga cttaagtagc ttcctatgag |
| 82381 | tccagagtta gcaagacata gttcttaaac tgcaatctga gagattccaa attctttgcc |
| 82441 | cattcaagtc aggctgcctc tgatgaaatc catttaggtt ttcttaagaa tagtaagact |
| 82501 | cataggcagg gacagaactg atcagtgata aaatatggga aaataggtct agtggcttct |
| 82561 | ggggaataga ggcatagaac catactcatt tccctagaaa acaaaatcgg ttacttttga |
| 82621 | acttgttaaa atttctctca gcttctggga ttttctttca ttggtattgt tttgcttaca |
| 82681 | ttcataagct gactagaata tcaagaaatg acctgaaaat aattgtacaa cttaaattcg |
| 82741 | agctatagaa gacagagaag gaaagaaaca gtggaactcc ttccccaggt ggatgactta |
| 82801 | ataaggataa ggaggagaga agcttgctgg aaacatcaag gagaaggggt cctggatatt |
| 82861 | cttacaatgt gaacatgggt ctcagattct agagtcccaa acactcagaa agagacacca |
| 82921 | ccagcagaaa ttgcaagctg attgttttat tggtataYtg aagttagagc atgatgccat |
| 82981 | gtttcacggc atttgaaaca ctgttatctt cttattcact tcctgaaaaa gacaagcaaa |
| 83041 | caaggaagat tacagacaac acttgacatg gcaggaaatg tctaatccct cactgggggt |
| 83101 | tactgcagtg atggagatgc aagcccctac tcttgccctc tatcccttct acagccccct |
| 83161 | tctccttagt aatattttct ttgttcactc tttaagccat tgcacataat gtgatttaaa |
| 83221 | gtgttgctta gatgagaaag agaaacaatt attgttatgt gttcatgctt tgctgctgtt |
| 83281 | ggttatgtaa gggctatttt tacaaagatg ggcactgcaa catatcatat aatagtgaga |
| 83341 | atgtgaatcc Mgccttaata tccagtgaca gggattggtt aaatagatta cggaacaacc |
| 83401 | aaacaataga atagtataca attgtaaaaa caaactaacc aRtaaacact gaagagatct |
| 83461 | aaatgaactc aKgagaaagt tttctaaaca ataaattttt ccagtgagta agacacaaat |
| 83521 | ctgaatatac agtatggtat aagtgcttgt aataaaagca ttatatatat ttatgtagca |
| 83581 | atatatattt caaaaagtag attccaaaga gtaaaaggtg ggggttttga gaaggataga |
| 83641 | agggaggaaa gatgagttag gctcttacta tttagctctg tacatttcgg ttcagtgaga |
| 83701 | ttagtttttg ttagcttaag catgtatgac tttaaatgta agtatactaa aaatgagata |
| 83761 | ccattttaca actgaaagtt caaggaccac ttagcaggaa tactggacct gaaagcctga |
| 83821 | tgtgaggcag gactgagcaa agcatgttca ggtgaaaact ttaaatggga gtctgagttt |
| 83881 | gaggaaagaa aatagagaga gccaaagggg gaacatggtt ctatgtcctc attgtcaacc |
| 83941 | ctacttaaga catgtaattt gaagaYagtg attctcaaaa tcagaattgc cttcaagagc |
| 84001 | ttctgtaaaa cacagggtca cagggtcttg atactctata caagaatatc tgaataacaa |
| 84061 | tttttagaaa tgtgttccag gacagggcaa tattaacagg ttttcagagg attcattggc |
| 84121 | acaatgaagt tggagaactg tagcagttgg agcctttgat ggataataaa ctggaatcgt |
| 84181 | acctgtcatt gaatcctaga ttactgagga gactgcccct gtggaggtcc ttgtgggcgg |
| 84241 | cccccttggg gaggtggtcc ctggggcttt ccaggaggag gtgggggagg accttgctga |
| 84301 | tggcctccct gttggggtgg tccttgtggc cttcctcgag gaggacgggg atggcctccc |
| 84361 | tgttggggtg gtccttgtgg ccttccttga ggaggagggg gatggcctcc ctgYtggggt |
| 84421 | ggtccttgtg gctttccctg aggaggtggt ggaccttgtt gctgctggcc tccttgttgg |
| 84481 | ggtggtcctt gtggctttcc ctgaggaggt ggtggacctt gttgctgctg gcctccttgt |
| 84541 | tggggtggtc cctgctgagg gccatcatcc tggttcccat caccagcaga gggttgagat |
| 84601 | tgctgtcctc ccaaaggtgg tccctgacgc tcctcatcta ggaactgctc agagtctcct |
| 84661 | ccatctgtgt gagttgaaac aagaagagct gagctcatgc tggaaaaccc tcctgtcttc |
| 84721 | atacctctct gtcttcacca cacggccggc ccctctctgc ctgacctgcc tctcaactcc |
| 84781 | caacctcccc ccttcccaag gcttcctaat tagaactcct cttaatccac ataagggtga |
| 84841 | tgaaaaattg aatttcttta ctcatggtcc ccagaatcaa ggttgggaga aaactgtttg |
| 84901 | tatctttggg gcattggtat tagccaattc ctgacaagaa aatggtgata agaagacact |
| 84961 | ggagaactca tcaatttttc agggaaaaat ggagacagag tttactgaga atttattggg |
| 85021 | atttacctga tattacgagg ggaacatctt cctggctgac atctagaaaa gaagtacagg |
| 85081 | atgatgggaa aagttacttc ctgaatcatt caaggctcat agtgttctac gaggataaag |
| 85141 | gacctctgat cacaccctgt gcatcccctt tgtgatctca tcagccactc tctgatgcta |
| 85201 | ctggaagtgg aagaagatgt aagggaaagc agtattgtta ctacactgag cgtcaaccag |
| 85261 | gaactcaaca tagaagggcc cctgtttgtc ctcttttgat tccccaagcc ttgatgagga |
| 85321 | atgaagatac aataggtctt ctgatcctcg gcatgaaaat tctgcagtcc catctgtttt |
| 85381 | ctcatcctcc tctcttccct ccactttcct cctctatagc atttgcctgt aaacccttag |
| 85441 | cgcttattta gttaaactgc tctgagtatt tcaatgacat atttggggat tcttctgccc |
| 85501 | tctttcatct gtaaatatgt tgtttatgtt tgcaagcctt ctcaacagga gccaccagac |
| 85561 | atagccactt ggatataatc ttacgcatgc cattcccagc acattgaaat actacattca |
| 85621 | ggagacagaa aaatgacaat gtttcactct ggttattctc taactctatg tagacagaga |
| 85681 | caagtgttta cccaattctc tgcctcggca gtagctctga catgtgttta tctccttcat |
| 85741 | taatgctcat ccactgtaca gcaaggccac catcatccct gtctactggg atcagtacag |
| 85801 | gatcttcaca gctgtacttc cctgaatctg tcttgcattc tccacatcct tcacagcagt |
| 85861 | cccatgtctc tatataatgc aaatctcact ctctcacacc cctagtacga attctttatt |
| 85921 | ggattctatt tgtgcaacaa ataaactatg aggccttgat gaggaatgga ggtacaatag |
| 85981 | atcttctgat cctcagcatg agaactctgc agtcccatct gttttctcat cctcctctct |
| 86041 | tccctccact ttcctcctct atagcatttg cctgtaaacc ccaatcagag tcacaatatc |
| 86101 | ttcccccaat tcatcttacc ttcatttaaa tcctgagctg agctgaaggc cagcagggcc |
| 86161 | actgacagca gaatcagaag catcttgcag gaggctctgg tgtcactccc aactttgtgc |
| 86221 | tgggagaaac gtgtcagctc cctttataaa gacaagcagg acaatggtgc atttgagctc |
| 86281 | cctaccaggt gggcctcctc gcctcagaga ctggcttctg ctttgcttac ttcaggtcaa |
| 86341 | gtgtatccct catttctttt gggactctag ctcagcagga agggttgggt aggatattgt |
| 86401 | ttgtgtctaa ttcctaaaag gcacaactat gacttggaca aatgttttga cggaactgtg |
| 86461 | tccaagcagt cggcacagtg tcaggattga actttagaca tcttttggtt ttcaatctgt |
| 86521 | ttggaaagac tgctattctg cttttcactg tgcttttcat ttStgtgtat gtgagtttgt |
| 86581 | gtgggtgtgt cggggggtgg acactgatcc tacagctaac agtgaagatg gtcaatatct |
| 86641 | ctgactgttt tgatagcctc tttccatctc tcatgatgtg tgtgcgtgga tattttcaca |
| 86701 | tttagctaaa tttttcatgt aacagagatg gtcttgctta tctgtgagat gtgtgaggac |
| 86761 | agcacactcc caggcacaca tatatgaRag aaaggKgctg gcagcctgct caggctacaa |
| 86821 | gaattgtcca aacttctgtg aatctcacta cactcaggca agccttggtg ctatacaaca |
| 86881 | caggcaagct aaaatatttg tattttaaaa atatctttag ggtttattaa agcaaaactg |
| 86941 | aactcagaaa ccaccaagga atttctagac ctgaacaaac tgaatatatg gttccctaag |
| 87001 | tgagccacac gatgtcatat agctaagacc tccactttct ccagaatatt ccatagaatt |
| 87061 | catcctctac tttatttctt ctaataaata ttgtaattgt catcactgat aattaaatgg |
| 87121 | ccacaattat acattcatct aagataattt gtaatacctg tccaagcctc aattgccttg |
| 87181 | attggtcttc tgcttttact cagtaattat gtcacctgtt tcatcagagt gttctttctc |
| 87241 | accaaggatt tttatctgta atattccaat actcattctt tctcattctt tttctcgcac |
| 87301 | ttatttgttg aaattttgta ttctgagtct accttgccat gtttactttt cgtcctatat |
| 87361 | cttttttttt ctccctacaa aggtgtcctt tatttatcct tctaagtaaa ggtgtgtggt |
| 87421 | actcctcact acctttactt tcatttttgt ctgtataatt tgacaataaa aaattaagtt |
| 87481 | tccaaaaagc cctgtcaggg tcctggtttg gtagctctct cctcagctgc tgattgctcc |
| 87541 | aaaaattaag tttccaaaaa gccctgtcag agtcctggtt ttgtcgctct ctcctcagct |
| 87601 | gctgattgct ctgtgatacc tagaaattcc ctgYtcatca attcataaaa tatactactc |
| 87661 | agaatcacca gctgcacttt ccatttcaga ccttgagtag gtcttgtgta atttttcact |
| 87721 | ttttgtgcat ggttagtctt tatttgagga tagcttggtt ggaggcatgg ctgagaagcc |
| 87781 | aggtgacaga aaatcataaa gacaaggccc tagacaggtg tgcatgagtg gatcctgtgc |
| 87841 | tgggcatatg agggtctggt atggctcttc tgaaaagttg cagataataa tacaagaagg |
| 87901 | cttcctagta gattagattt tgctacagac gacattgttc ttgtaaggga cccaggcaga |
| 87961 | ttggtatttt ccctggaata aatatagacc tatttcaggg gggaatgcat tcttaattgg |
| 88021 | caattcttct ctggccctgt gggaagtaga tcagctctag ctgggccgta tgagttgtat |
| 88081 | ctcaggcaca gagtagttgc ttgagcccag cagctcccaa tgcgttatgg agattattgt |
| 88141 | ctctggtttc accaggtact gtttcccata ggacctggag atgagcagtc ctgcctcggt |
| 88201 | gacttcaggt caaccatcat gtcaaaaagg aaggtcagag aaaaggaaca aagaggagtg |
| 88261 | ccaggcagat attgcctgat gatgtggggg actgaggaag ctgcaggcct ggtgcaagac |
| 88321 | aggtttctcc ttggcagcag agtacaggaa aagaacagga aatcctccta aaggtcactc |
| 88381 | agctcaggcc tgctccctaa cctgctccaa tgtcagcaat cagcgagaga ctagtgtatc |
| 88441 | aggaaagcat aaatatcagc tatgctctca ctagacacat caaaaacaga ggtaatggaa |
| 88501 | gagctgaaag ggacactgtc tgtgagtgaR ggtgggtgaa agggagacgc aggactgctg |
| 88561 | caggagcttg ggtacctaga ggtgggtggc agccctcagc accacacttg ccttctacta |
| 88621 | tagatgttca tattctgagg ccctgaaggg ccagtcttca acatatgaaa ttttaggatt |
| 88681 | catctctcat aacacatccc aacattttct cggtcactga caggtgttta cctagtggtt |
| 88741 | caaaaatcct cgctccctcc aggaacttat gcattagccg atctaattgc ctccactttc |
| 88801 | aaaatacgtc ccaaatatga ccacttgcct gatctttact agaaggtgat tgttcagcca |
| 88861 | tgatgctctc tcctggagga ctttaccagc ctccagctgg tcttggtaat gttcttcccc |
| 88921 | cagctcatcc catatcagtt ccctcacaac aatcagagtg atcatttaaa agtagaaatt |
| 88981 | aggttatatc acatttgtgc ttcaaatctc ctgttttttc ttctgcactt gtgataagat |
| 89041 | ccaaatttca tgcctcagct ctttcacacc cacatgcctc actctttttt gaaaaccagg |
| 89101 | aagctctctg aatggaacag aaagtagaat cttcttcgac ttcaccatgc tctagtttgc |
| 89161 | ctggaacact aaagatggtt tgccaggaaa gatgaacatt gRcagaggga ataaacttct |
| 89221 | ctgagagtca gtgagtttca ttgggtcgct gtggtctggc agtgttatga attggcagaa |
| 89281 | ttattctatt aattgcacca actaagcatt ctgataatac agttagcaaa taaacacaat |
| 89341 | agtctctgag acttcaactt gctttgactt gtctaaaaag tttggtgaca ctattatggc |
| 89401 | caattaacta ttaaaaatat cttctatatt tttaaatttg atgttagtgg tccataaata |
| 89461 | tatatatttc acaattaatt tagcatattt cttttttttt tttttgagat ggtgtcttgc |
| 89521 | tctgtcaccc aggttggggt gcagtggcat gatctcagct cactgcaact tccacctccc |
| 89581 | gggttcaagc aattctcctg cctcagcctc ccaagtagct gggattacag gYccctcgcc |
| 89641 | accatgactg actaattttt tcccatctct actaaaaata caaaaaaaaa attataYcta |
| 89701 | aagaaattaa gtcacatggc taRggtcact gtagtaaatg aggaaaaatt aagttgattt |
| 89761 | tctcttctct ttgtaattcc agtttaaccg gatgttgtcg atgttgtttt caatttagaa |
| 89821 | aatatagcaa attgttcatt agccatgagt atcactctgc tatccaccac tttctaagaa |
| 89881 | ggcgcttatt ttagaacaag gctaacacta aattacagga aattaagtaa ttgtggcaaa |
| 89941 | ctagagaagc atagtatgta taataggtct tactcagtta gatccacctg tcaaggctga |
| 90001 | gtccctttct ggagtctcta gagaagcacc cattttttgt ccacattcct tggccttctt |
| 90061 | ccatcttcaa tgccagcaat agccacttga gcctcccatc acatagcatc acgtcgccct |
| 90121 | tgcactgact cttctgcctc cctcttctgc agttaatgac cttatggtta cattgagtcc |
| 90181 | acctagataa tccaggataa cctttctaaa ttcaggtcaa ttgattcgca aatgtaattt |
| 90241 | ccctttgtca tgtaagttaa tatattcact tgtctgagaa ttaggaagtg tgtgactttg |
| 90301 | ggagactgtt aatcttcata gaacacacag gatggacacc ttacccatct agacctggtt |
| 90361 | tggctcagtg agagttggag agactcaccc aggcttcttc cagatatttg catgagaata |
| 90421 | tgaatgagtt tatgtataat tagttgttat acaaatattt gtgacttaaa gtatatatct |
| 90481 | atgcaaaagc gtaaatcaca aactgagatg gcaaaaattt actttcaata accattgaca |
| 90541 | cttaatgcat tttttgatat tttagagctg tcatattttt tgttttttat ttatactact |
| 90601 | tttgattgtt ttctttttcc tgatctccta tggggccact gaaagtgttt tataattcta |
| 90661 | ttttagttga tccgtataat ttttgagtgt atctctctga acagtgtttt tagtgattct |
| 90721 | tctatgtttt actttacatg tacatagctt ataatctagt ggtgtacact ttttaccagt |
| 90781 | tttagtgaag tatggaaacc ttacatccct ttataatttt taggtcaaaa tgaatttatt |
| 90841 | atttttcagt tttgtaggtc agaaatctga actgaggttc actgagttaa atccatgtgt |
| 90901 | caaggctgag tttcctccta gaggctctag agaagaatgt catttttcca catttcctgt |
| 90961 | ctctcttcca tcttcaatgc tagcaatggc tggttaaaac atatgaagaa aactcaagtg |
| 91021 | aaaaaggaag atatattgtg tttgcccact ttcccccttt ctSttatttt tacttcattc |
| 91081 | ttgatgttcc acaaatttct cttctattct ttcctttatg tttagagaac ttcatttggc |
| 91141 | tattatttaa agataggttt gttggaaaga aattctccta ggtttctagc ttttcttttt |
| 91201 | ttcttagaat gcctggattt cctttacatt aatgaaggat atttttattg aacataaaca |
| 91261 | tctggtagac catttctact tttgagcact ttaaatattt gtgctccttt ctactgttat |
| 91321 | ttctggtttc tggtSataat aaccactatt atttatattg cattttccct ataactatga |
| 91381 | tgtcatttcc ttctttctgc tttcaatatt ttgtctttaa ttttcagaaa ttggacaatg |
| 91441 | aattttcctg gaatggattt cattggttgt attctggttg ggatttgctc agcttcttga |
| 91501 | atcYgtaggt ttatgctctt tgctgtgttt ggaaaatttt tgatcattat ttctctgagt |
| 91561 | cctttttcaa catcaacctc ttctgctttt ctgaaactca gaatgcatga atagtacaYc |
| 91621 | atttgttata gcctttcata ttcatgatat gatgtataat tttttcaatt attttaccct |
| 91681 | gtattgttta gagtatataa tttctattga tccaacctca agaacactgt ctttcctcca |
| 91741 | tcattttctt tatgctattt atcatgccca ataaaggttt tttaagacat tatatttttc |
| 91801 | atttctaaaa tatctgtttt cttaatttat aatttataat tcctattttt ctgctgacaa |
| 91861 | gtttttcctt tccattcatt ttgagtgtct accttgacct catgaaggat gggtacaaca |
| 91921 | gctcctgtaa agtctttggt cattttaata tctaagtaat ttcaaggttg taatttcttg |
| 91981 | attgtttcat tccttgagaa atggtcgatt ttttgtgagt tgttttgagt acattgagta |
| 92041 | attttctgtt ttatcttgca cagagggatt ttctcttttt gcaatacaca aaaagcccaa |
| 92101 | taagggtagg ttcggacatt ggagggtgaa ggcactcatg gtaaattatg ctccaccaac |
| 92161 | tggctcaaat ggccagggag tttgtcccac taacattttt ggtggttttc cctgggtcaa |
| 92221 | tctctctggc acttatgaga taatatttta agtatttaat atcacaccat acatacagat |
| 92281 | ataaagacaa taaaaagcag tgcatcgaat gggtccaaag ggccctatcc acaaagtctt |
| 92341 | cctaactttg gtgtgtgtgt gtgtgtgttt gtgtgtgtgt gtgtgcactg tttgttcaat |
| 92401 | cctcatcagt tgaactcagg cacttctttt tcctttttct ttctaaacct gcttgttaga |
| 92461 | ggctgtcaca gtagccagaa ggccctgcta tcttctattt gagtatttat agaatgcagg |
| 92521 | aaaaaagagg gtaaactata aagaacgact gtaggcctcc ccggtgcagt ggctcatgcc |
| 92581 | tgtaatatca gtacttggag aggcagaggt gggaggactg cttaaggcca ggagttctag |
| 92641 | accagcctgg gtgacatagt gagactcctc tgtctttaca aaacactaaa aaattatcca |
| 92701 | ggcatgatgg tgtgtgcctg ttgtcctagt catttgggtg gctgaggtgg gaggaaaaga |
| 92761 | agggaaggaa agaggagggg atgggagggg aggggagggt tggctttttt tttttcctct |
| 92821 | tggcttcttg cccttctttc tagtgcacct cccagtaggg tctcttttgt atttcctcac |
| 92881 | atgctattgt cctccatccc tcagcacccc ttcccccaac ccccagaacc aagtagaatg |
| 92941 | gtgatttgtg cacatatatc caacagggct atactaattt gagtccctac tctccttgaa |
| 93001 | gctttgcaat atgttgggat ggttctgtag ggtctttggt gtcccttggg tcaggaagat |
| 93061 | ctcaggccca gcccgaatcc ctaaagcaat tgacatcaga atagaggtaa agaagttatc |
| 93121 | agggaaatat agagaaagtg tcttcaggcc agtatgcaag acacatttac tttcaaataa |
| 93181 | gcattattgc cactactcgg tggttaactg aataagcatc tgaatgtaac aactttggga |
| 93241 | gatccttaat cttttcacct ttccaccaaa ggcctgtaac aactttggga gatctttaat |
| 93301 | ctgtctacct aatgttatct ggagaagaca gctaaatcta aaagtgtcat ttggaagtct |
| 93361 | gtttcagttc taccagtggt actcaatttt caaccacttc ccaatcctcc tcattaatga |
| 93421 | gcaacataat agaagaatat ttatttacca attacaaaca ttttagtcaa catgttagct |
| 93481 | accagttccc atggactttc agcaaatcct attaatcagc ttgtaggatc cttcactttc |
| 93541 | tcctcagaag gccaggtctt tgggctgtgt tccatcctcg ttgccaaaat tattataagc |
| 93601 | taacaactta gcttgtcact tgttgtactt gcagaaggca aaagatggtt tattattcac |
| 93661 | tgaagtacta gcagtagtca gaggaatatc atcgtactat ttcttcaagc ttcaRtcccc |
| 93721 | tcacagggat aagatgagaa actgatgtta catgtgcaag tagtggtgtc tatcaaaaga |
| 93781 | gaaatctcaa aattaggaaa ctccagtctt aaaaactttg atcactgtga gtaacctgtc |
| 93841 | catcttgcct tctgaagaag aagctttaca ctggaatgta agcaaatatc tctggagaga |
| 93901 | agaagaagaa gagaagagtc tttatcttta aacaaatctg ccttYtgacc aagagggata |
| 93961 | ccctatttct aaatcccaag gctctttgcc atgacctggt cttgggcaaa gtgaacgaga |
| 94021 | ctcctagggc acaaaacgta aaaaggcatt caataggttg cagggcccta aaaccaagtg |
| 94081 | gctcctcaaa ctttgcaccc tgcatgcctt gctcacctga tcctagttga ggcccttctg |
| 94141 | tttgctcatc aaacatactg tttgctcaca agataagagc tagaccatgc agaagtgcaa |
| 94201 | tacattcatg gagaattttt ttcccaacat ccatYcccaa gatcaaggtt acacagttgc |
| 94261 | caaagattgt agagtattct gaatatatca tctattttct tcctggacag aactaaatcc |
| 94321 | atcagaagta acttttttaa aaaatgtttc tttcttttgc atacaatttg tttttatttt |
| 94381 | agattaagag atgagtccag aggtaagggg gacaggagaa ctgtgacatc agggatgaca |
| 94441 | ctaccctttg agagcactgg agcagttaga aaggattgaa agtgaataat cttatttcat |
| 94501 | aaggggtatg tcattttatg attcaaatta agtctgtgat tatcacttag caaaattgct |
| 94561 | aaaatcatta tcagaatatc ccatatcatg ataggaggaa gaaaaagaag taaaatctaa |
| 94621 | aattgttatg gaggtaaata gaagactgcc ataaacaagc tatctacaga attatggggc |
| 94681 | ttaggaattc acagtaccct ataaacaaga acacagcttt gagaatttct atactctaat |
| 94741 | ttttaaaaat gaagtatatt ttgtgaattt ttcacccaca aattatttta actggaagcc |
| 94801 | atcacagctc attaatcagc aattggtgaa ggactaaagc agcccaaatc attcctgtct |
| 94861 | caaagtaagg cctgcttgta agcaagtgtg aactggggat gtatgtgcag aaatcagagc |
| 94921 | ctaagtatgg taagctggac atttcagaga taaggaaggc agctggtgtg tgtgaaatct |
| 94981 | tatatcaata actgatttta ttttttcagt cctaattgct attatggaat ttgtgctttt |
| 95041 | cattattaca tcttcacaaa gcatgatttt taactttaga acaatttaca ttttatgctg |
| 95101 | gataattctg ttgtgaggga ctgtcatgtg cattgtgaga tattgagcag catccttgtc |
| 95161 | ctcgtacagt tatcaggtaa aatacagagt gtgtcagttc aatttgaatt tcaaataagc |
| 95221 | aacaaataat tgtcattgtc tgtgtatcca attatcaatg ctgcaggtct gtaattggtc |
| 95281 | ctccatagca cacttacatt atgatgtaaa ctcatattat ggttatcaca agtaatactt |
| 95341 | atttataaat ttattcttgt tgttttttct tttgtctgaa atgtaaattt aactaggcct |
| 95401 | cctatatttt tatttgctta atctgttaac attacaatag atgccagcag catccgtcca |
| 95461 | attttaactg aaatgtctcc aggcattatt aaatgtcccc taggggggca aaattgccct |
| 95521 | gtgtggagaa ccactgtcct aaagggattt catcctttgc acatggaaat ctcgtggtct |
| 95581 | attctctgga tattttaatt ttattcacac atttaaaatt tcgttttagg cctgcaatgt |
| 95641 | ccaggttatt tttctagggt ttactttttg ttctatcgat taccatataa ttaacttttt |
| 95701 | aaaatacttt ctgcaatgtt gaaaatttgt cttttgtctt tactgctgaa aattatatta |
| 95761 | tttgcttgca atatgttctt ttttttctta gagagaacgt aattgttctt caaaatctgc |
| 95821 | ttccaattat ttcattttaa atatttatta Ytgttacctt aaacttgcga ttgtgttctt |
| 95881 | ttatttaacc tgctattttt acatgctttc tcattacatt tttaagagga gacgttctta |
| 95941 | ttaaattcaa ttctgtaaac tgattggtta tccctgattt gtttttcttt tcaaaacttt |
| 96001 | atttattttt ctttctttac acagtgactt tcatcaaatg agatcctgta tttgttacta |
| 96061 | tcgtcttggt cttctatatt tttatttact aaacctggta actctacact agatgccagc |
| 96121 | agcagtcctc caattttgct tacttaaaat cttactgatt ttttaatacg tgtccttgcc |
| 96181 | ttccttacac agttaaccaa ttgagtaaaa ttttattgtt agtccaaaga ttcagattct |
| 96241 | acagtacaag tctgttgtag atggtcaaga ttgtaacatt tttgatggtc aaagttgctt |
| 96301 | agtgttttta actgaaaatg actgaaaata ttaacagaat agggcttata tttggaaata |
| 96361 | actcttagtg atgtacattt caattgactt tgtttcatat cctcaaagtt ttcactaagc |
| 96421 | tgtagactct actaactatt ttcatttgag ttttcctttc tagttttaaa ttatcaattg |
| 96481 | atcatcattt ttaggatagg aactaaatcc ctatgtgttt gtacatataa atctgatttt |
| 96541 | atatacactc tatacacata catagatatt ataattcttg atgtatttct attctattct |
| 96601 | aatgcttatg caacatatta ataaaattta aagatagagt aatctattat attgatcaca |
| 96661 | gtgcctaatg aaaaatgcat tttcaatata ttagaatttt actattaaat acataatctc |
| 96721 | catatatggt gcaaacaagg tagcaatctg ttctgcttaa tttaaatcag caagtatttt |
| 96781 | caataatgct caatatatca tagaacataa tgagaaaata agaggtaggt gaaagaaaca |
| 96841 | aatgtgtagc cctcctgttc ttcttccttt ttttctttct gagaatatca tcactcatat |
| 96901 | caggcatgtt tataaaaatg agagattatg tcctttttgg catacttcat cttcttcagg |
| 96961 | acacagagag aagcttgctt cagtttgctg tcccgtaaaa ttagaagaaa tgaatggcca |
| 97021 | gatggatgga aaaataccag cacactacta aaagtccttg ccgcaacgaa gtctagaaga |
| 97081 | ggacaggcca atgttagtat gatactgatg aaataatgca caataaagag gagtagaaat |
| 97141 | gaaattatag cttttatagg ttttacatgg gcttctgtgc tgggatccct ggaataaata |
| 97201 | ccatgtagct tcatctgcct ggtgtggctc cataaggaga ggattaagag aagaatggaa |
| 97261 | gccagtgttt ccacaaaagg aaggatgaac atcatctgaa ggagaaacag agaagtgaaa |
| 97321 | tcatgcatgg tatccatata attaaatgtc aagttgcctt ttattgttac ccgttcttag |
| 97381 | attaaactat taattattat ttccttcaga atggaagttg tgcagaaaga gatcgttgcc |
| 97441 | tctagtacaa taaagagaag caccttgtga attttccatt tcatccagag gaaaatggga |
| 97501 | ttggagaagt tggctacctt gaggaaatag aagacactga ggcaagtggt acaggacagg |
| 97561 | cagaaatagc tggatcctgt ccagagaatg tcaaaactta ctgcaagatt cttagaatca |
| 97621 | ggcattttct catagcctat attgaaagag gtagctaaaa ttgttatcta caggaaacat |
| 97681 | atcctggaga tggccaagca gttgagaata aaatcaatca gggagacctt tcagttcctg |
| 97741 | attcagtcaa tgatattaac caatacaatg aatcaatttc ccacagtccc cattatgaat |
| 97801 | tacactacaa gaattcttat aaacagtgtc tcccaaatgc ttgacatgcc tctacatagt |
| 97861 | gaatctctgc cttcaataca ccattggctg attgatccta gctgcactgt tatagattta |
| 97921 | tatatccaaa ggagtgactt ctttctgttt tattgatggc cattggtgta gtctggagag |
| 97981 | atccaaggtg tgttctaaaa ttagatttca actaaattgt aaaatataca agtttttttc |
| 98041 | ccttgagggt atcagctagt gattttcaag gcaaatatta tattccctgc tactattgac |
| 98101 | tctggttgta ttttacatgg aaagttctgc attcaaattt gaaccctcat ttgttaacat |
| 98161 | gcaaaattga agtagattct cctttatagt tttacaatgc catcttttgc ttaaaggtta |
| 98221 | ttcatttgta tgatgtatcc ctttgattca tctaccaccc tgggctagaa attctaagcc |
| 98281 | cccatataga aaaatgcagt atatatttag agtttatgat cagcatattt cttcattatc |
| 98341 | aacctcacta atgcagatta tttataaaat atgcaaagtt taagaaaatg gagaaaactg |
| 98401 | ctctctgata taataccaaa tcttttagag agtatggaag tttttggcca ggcacggtgg |
| 98461 | ctcacgcctg taatcccagc actttggaag gccaaggcag gtggatcacc tgaggtcagg |
| 98521 | agttcgagac cagcctggcc aacatggtga aaccctgtct ctactaaaaa tacaaaaatt |
| 98581 | agctgggctt ggtggcatgc tcctgtaatc ccagttactc gggaggctga ggcaggagaa |
| 98641 | tcgcttgaac ccaggaggca gatgttgcag tgagccgaga tcacactact gcactccagc |
| 98701 | ctgggtgaca gagtgagatc cgtctcaaaa aaaaaaaaaa aagagagaga gagtatgcaa |
| 98761 | gttttaaaac atacgataca tactttggca cttcttctat tgatatctta ggagtctgtg |
| 98821 | tactctgtcc ttaaatctag aatggtttgt gactaattca actaataaaa atgctataat |
| 98881 | aattctaaag ctaaatcatg aaaagccatg cagttttttc ttaatcactg caatatttac |
| 98941 | tcttggagtc ctaagccaca tgtaagtagt ccaactactc tgaggtcaca atgtccaaac |
| 99001 | ccacatgcag aggccatgtg caggccctct ggctcattat cccacgtgag cccagctttt |
| 99061 | acgtcctttt accccaggta ctaaacctat gagccaagaa agcgatattt gacctgtctg |
| 99121 | atacggtaga tactagccac atgtcacacg tggcttttta aattcacatt aataaaaatt |
| 99181 | aaataagatt aaaagtacag agcctcagta ttactaacca tatttcaaat gctcactagc |
| 99241 | cacgtgtgac tattttatag ccaacataca taggggatat ttctatcatt gcctatcatt |
| 99301 | gacatttcta tgtatttctc agctccgcct tagataagtt cagcccctgg tcacttgaat |
| 99361 | tgcccccagt tgttcataac tttctggcta aaggcttaga cattctggaa caaaaagaag |
| 99421 | ccatccctgc tatgtcttga acaaaaccct aacctacaga atctttaagt gaattagtct |
| 99481 | attcttgcat tgctataaag aaacacctga aactgggttt ctgtaagagg tttaattggc |
| 99541 | tcatggcttc acaggctgta caggaagcat ggcggcatct gtttctgggg aggcctcagg |
| 99601 | aaatgtataa agatggcgga aggcaatggg gaaatcagat acatcttcac atggttggag |
| 99661 | caggaggaag agaaagatgg gggaggtgcc ttacactttc aaacaaccaa atcttgtgat |
| 99721 | aactcactca ctcactattg ccacaccacc accaaggggg aaactgcccc ccataatcca |
| 99781 | atcacctccc accaggcccc acctccaaca ttaggggtta caattcaata tgagatttgg |
| 99841 | gcgaggacag aaattcaaac catatcaaca agcacaataa aatggctgtt attttacacc |
| 99901 | atgaagtttc tgggtgattt tattatgcag caatagagca tcagaataga attatttagc |
| 99961 | atatgaatgg tatcaatatt ttgtcatttc taatttccat gtctgttttt ctcttctatc |
| 100021 | ccaaataaag gtatccatag catctttatt ttcctcagtg taaggggaac ttgtataaat |
| 100081 | tttctataat tttttcatac gttgagctga acttaatctt aattctgatg aggtcaaacc |
| 100141 | aggaaaggcc aattagtcat gttctttgtg taatgcaact gctgcatttc cctcccaaag |
| 100201 | tagctagtcc aaaatttagt gacattcaaa accaatggag atataatata ggcctaagag |
| 100261 | gagtcaaaga aatgaatgag ttggacagcc caccaaaaga tacaccctag gtaaattcac |
| 100321 | aaagaaaact ccatgactaa aagaaaggct aattgtgata ggcaaacatg aaatgcaaac |
| 100381 | aaaactgaaa ctgaaataaa caaagggaaa ttcaggaaaa tattatatgc tgaaaaaggg |
| 100441 | ttattttatt tgtcttattg aatatcttct ttgttccagc cctctaggag gtgggatgaa |
| 100501 | gcattaaata caactatttt taaatgccct taataagttg acattctgaa aagatgaact |
| 100561 | ctaaacatat aactaataaa atatatatca tacagatata aaatatagtt tataagaagt |
| 100621 | tgcaaatata agtagagagg gtcaatagaa agcatgtcag aagaggggtt gaaattttaa |
| 100681 | ataaggtgct tagggaagga ctcattgaga aggtgatgtc tgaacaaaga cctaaagtag |
| 100741 | gtaaagaaat tatccatgtt ttgtctgggt aaagtacatt ccagacacac ggaacagcat |
| 100801 | tatcagtgtg ctaacattgg atgtacctag tgtgtctgag gaagaacaag gaaagcagtg |
| 100861 | tgactgttgc ggagatgaga gagtgagacc actaagagtg aagtctcaga aagaagaggc |
| 100921 | atgtgcccag aaggcagcac atacagtcaa aagagattat tttagaacgt taatatttaa |
| 100981 | cgcctgccct gctgtgtttt gtacttgcgt ggtgcctact gctcctctct tgtggctgat |
| 101041 | ttctcccttt tggaatgaga atgtctatcc aatgcctgta caacaactgt atcttggaag |
| 101101 | gaaataaatt gttttttatt tcacaggctc atagctggaa ggaacttgac ttccagtctc |
| 101161 | aaatgagact ttggacttca gacatttgaa ttgatgcagc aacgtgttaa gaattggggg |
| 101221 | actattgttg ggatggaaag attacatttt tttttatgtg agaaggacac gagttttcag |
| 101281 | gggaccaagt cagaatgcta tggtttgtac acggtttgtt tgtccccatc agaactcata |
| 101341 | ttgggatttg atccccaaag tagtggtgtt gggaggttga gcttagtgag aagtgtttga |
| 101401 | gtcatagggt tggttccctc atgaatgcct tggtaccatt ttcaatgtag tgagtgagtt |
| 101461 | ctcactctca caagactgaa tttgttctag gggaatgagt tgtttttcat gagagtgaat |
| 101521 | tgttataaag ccaggatgcc cctttggttt attctctctt cacatgtgcc cgcttctgct |
| 101581 | ttgaccttct ccaccatggt ttgacccagc acaaaagctc tcagcagaaa ctaagcagat |
| 101641 | gctaatgcca tgcttcttgt acagcctgcg gaaccatgaa ctaaataaac ctcttttatt |
| 101701 | tataaattac tcagcttcag gtattccttt ataccaacac aaacagacca agaaattgag |
| 101761 | gaataatatt tataaaatta tttcagtgaa ttaaattaga taaaatggac aaattccctg |
| 101821 | aaagaaatta ctgaatcagg cacacaaaga aatagaaaat cttagtgtct atttttaaaa |
| 101881 | attgaattct taatttaaaa ctttcacaca aagcaaggga tgacctgaat gtttattttc |
| 101941 | atcacatatt taagaagaaa aaaataccaa cctacataaa tttttcaaca aagtctcata |
| 102001 | aaggttaact ctgtgagctt tatttcaaga aggaaagtgg gaatctacca gaatcataag |
| 102061 | gacacacaga tcccagggag gggaacatgg gcaaacagcc cccatgatgg cacccagtta |
| 102121 | aaaaagtgaa tgaagctcca gtaggtgaga gaggcagaga gcctccctct gtgactcacc |
| 102181 | tttccactgg ggatctgagc aacccagtgc tcagagggaa gagtaccttg tttctccaaa |
| 102241 | gccctggaga taacttggag aaaggcttag agacattgta agggaaagac actgggaaaa |
| 102301 | ggttcaggcc ttttctcaga cccaggactg agagcaggat accattttta attcagatgc |
| 102361 | cacaaagtca atcattcttt ggtaactaag cagcgtagct gtgcaggcat tttagtcttg |
| 102421 | ggccagatat tggagcacct gctttagagc agggtgggga cctccatggc cagaattgtg |
| 102481 | gggaaaaaaa accttagcag caaacactga aactgtgcta tcccccatca caggcctggg |
| 102541 | gcaggagggg agctgctaca gccagtttat cctatgtgat gagacttgca accaggcagc |
| 102601 | ttagctactt ggaactagtc tgtgtgtgtc attgctgggt accccaccct gttcccctgt |
| 102661 | gattgtggtg cagcagactc tcctccactc tacctctagg cagaaatctg ggcatttgga |
| 102721 | tcactcattc acctggttca gcagcttcat ctgctccacc cttcttgtgc agagatccta |
| 102781 | gaatagggag gccctctctg cttcatgcct aggcagatcc caggattttg gagcacctgc |
| 102841 | tagcaaggtt cagcaccctg aaccacctaa ccttccttga cgtaggtcat ggtgcaatgg |
| 102901 | ggccctctct gctccacagc caggcagaac tctagatatc tgaagcaccc attcacctgg |
| 102961 | ctcagcagcc tatcctcccc tacccttcct gtgcagagat tttgatgcag ggaggtcctc |
| 103021 | tctgctgcat accctagcat tcaaagcacc tgctcacaca gactggcaac ctgtgctgcc |
| 103081 | tcacccttcc cgttcacaga tccatgtact gggctgatcc tctctgtttt atgcccaggc |
| 103141 | aaatatccaa gcattaagag cacctgcctg cctagttaag aagcctgagt caccccattt |
| 103201 | accctgtgca gaaatcttgg ttggtggggg tgggttctgc tccacaagca ggcagatttc |
| 103261 | caggtattca gagcccccac tctcctggat taagagttta ggccaatctc tccaatatcc |
| 103321 | ctgtgcaaag aacttggagc cgagggggtt acctagctcc acccctagac agaactctgg |
| 103381 | tcacttggta gccactcagt agattctccc tcagtgttgg tgcttgtgtc tgccattgga |
| 103441 | agacctatag gtggacctgc cttgtctggc cccacccacc ttgctcccca caacccccaa |
| 103501 | ggctgagcag ggagttcaca ccactgtgta ctccatgagt cagaccatta tctgcagcaa |
| 103561 | cagagagttt cttccagtaa acaaggatca aatatataac catcacattg tccaaaactg |
| 103621 | gctcttacct ataagtgcca tctactggct tgtaggttga accgcacagt ctaatataaa |
| 103681 | acctgcaacc tgtaagtgca taaggctata gaagctgggc aaaagaccta cccagaattc |
| 103741 | tctaaagtca tacctcctaa ggagggaggg ggaatggaaa gggaaagaaa aaagaacaat |
| 103801 | aatattatag ggaaataaag aaaaaaatcc taccttatgg aaataattac aaaaattaga |
| 103861 | agtgccagta ttgccagagg aggaggaaag agaacaagaa ttctaccacc ataaaaaaag |
| 103921 | tgaatgtaat gaaatcaaca ccatcaaagg agcacagtac ctctccagca atggtcccta |
| 103981 | accaaaacaa aagctcagat atgacagata aaaaattcaa agtatagatt gcaaggaagc |
| 104041 | tcaatgagat gcaagacaag gctgaaaatg aatacagaga gatttctaaa gcaatccagg |
| 104101 | aaatgaagga agacataaac atcttacaaa gaaatcaatc agagcttctg gaattgagaa |
| 104161 | acttacttaa ggaattataa agtacaattg aattcatata aagtacaatc tctcatagac |
| 104221 | tggaccaagc agaagagttt cagaccttga aaactggtct tttgaactaa cccagaaaaa |
| 104281 | aaaattaaaa ataatttaaa aatatgaata cagtcttaga gcaatatggg attatgtgaa |
| 104341 | gcaaccaaac tcataaactg ctggcattcc tgagaaagaa ggagaaatag ttaataacct |
| 104401 | ggaaaacata tttgaggaaa taatttaaga aaatttccct aatcttgcta gagaggtaga |
| 104461 | catacagata caagaaatca agaggacacc tgactgatgc tataggaaat gaacatcacc |
| 104521 | aaagcatata gtcgccagac tatccaaggt ctacactaaa gaaaaaaatc ttaaaggcag |
| 104581 | ctagagaaaa agatcagatc acatacaaag ggaaccccat aaggccaagt atatagctca |
| 104641 | gcagaaacct tacaagccag gagagattgg aggcctactt tcagcattct taaaggaaag |
| 104701 | aaaattttcc aaccaataat tgtatattcc atcaaactaa cctttataac tgaaggagaa |
| 104761 | ataaaatctt ttatagacaa gcaaagggta aaggaattta ttaccactgg accagtctta |
| 104821 | caagagatcc ttaagggagt tctaaacatg aaaatctgct accaaaatag cacacctaag |
| 104881 | cacatagccc aagaccctat aaagaaacca cacaatagaa actacaaagc acccagctaa |
| 104941 | caacttcaaa atagaatcaa aacctcacat agcaatatta gccttgaatg taaatggtct |
| 105001 | aaataccctc acttaaaagt cacagagtga caaattcaat aaaaaacaaa agtcatcggt |
| 105061 | ctgctgtctt caagagaccc atctcacatg taatgttact gttaggctca aagtaaaggg |
| 105121 | ttgtagaagg atctgtaaca caaatagaaa agaaaaaaag aaagaatcac tattcctata |
| 105181 | taagataaaa cagattttaa accaacaaca gtaaaaaaat gacacagaaa ggcactacat |
| 105241 | aataataaag tgttcaattc aacaagaaga cttaactgcc ccaaatatac atgcactcag |
| 105301 | cattggagca ccaagattca tgaaacaagt acctccaaac ctataaaaag atttagatac |
| 105361 | ccactcaata atattggggg acttcaacac cccactgaga agattggaca gatcattgag |
| 105421 | gcagaaaact aacaaggaaa ttctggactc ttgaaaaaac gctcaaataa ttaggcattg |
| 105481 | aaggaacata cctcaaaata ataagagcca tctatgacaa aactacagct aacattacac |
| 105541 | tgaacatgca aaaactgaaa gcattcccct tgagaactgg aacaagaaaa gggtccccac |
| 105601 | tcccatcact cctattcaac atagtattga aagtcctagc caggacaatc aggcaagaaa |
| 105661 | aaaaaaggca tccaaataga aaaataaaag ccatagtcaa actatatctc tttgctgatg |
| 105721 | tgaatccata catacaaaac cctaaagact ctgccaaaag gctcctggca cagataaagg |
| 105781 | acttctgtaa agtttcagga tacaaatcaa ttcacaaaaa tcagtagcat ttctatacaa |
| 105841 | taaaaatctt caagctgaga gccaaatcaa gaatgcaatc ccatttacaa tagcctcaaa |
| 105901 | aaaaaaaaaa aaaaaaaaaa caactaggac tacagctaac caaggaggtg aaagagaact |
| 105961 | gtgaaacact gctgaaagaa atcatagatg acacaaacaa atggaaaagt attccatgct |
| 106021 | catggatagg aagaatgaat attgttaaaa tggttgtacg gcccaaagca atttacagat |
| 106081 | tcaactctat tcctatcaag ctaccaatgt cattttgcac agaactagaa aaaaaaaact |
| 106141 | attttaaaat tcatataaaa ccaaaaaaag agcccaaata gcaaaagcaa ccctaagcaa |
| 106201 | aaagaaaaaa gccagaggaa tcacattacc tgacttcaaa ctatactata aagctacagt |
| 106261 | aaccaaaaca gcatggtaat attataaaaa cagacacata gaccaatgga acagaataga |
| 106321 | gaacccagaa ataaagacac acaactacag ccatctgatg tttgacaaaa ctgacaaaaa |
| 106381 | taaacaatgg ggaaaggact ctgtattcag taaatggtgc tgggatatct ggctagccat |
| 106441 | aagcaaaata attaaactga acctttacct ttcaccatat acaaaaatta actcaggatg |
| 106501 | ggtcaaaaat ttaaatgtaa gactttatac tacaataatc ctggaagaaa acttaggaaa |
| 106561 | cacaattcta aacattggcc ttgggaaaga atttatgact aagtccccaa aagcaattgc |
| 106621 | aacaaaaaac aaaaattgac aagtgggacc taattaaact aaagaatttc tgcacacatt |
| 106681 | gtataaacta tcaacagagt aaacagacaa cctatagaat gtaagatttg caaactatgc |
| 106741 | atgcaacaag ggtctaatat cccaaatcta taaggaatgg aaacaattga acaagcagaa |
| 106801 | aacaaataac ccctttaaaa acgagcaaaa gacatgaaca ggcacttttc aaaagaagac |
| 106861 | atacgagcag ccaacaaata ttttaaaagg ctcaacatca ctaatcatta gagacacgca |
| 106921 | aatcaaaacc acaaagagat accatctcac accagtcaga atagctacta tcaaaaagtt |
| 106981 | aaaaaaaaaa aaacagatgc tagtgaagct gtggagcaat gggaacgctt gtaccctgtt |
| 107041 | ggtgggcatg caaattacgt cagccactgt agaaagcagt ttggagattt ctcaaagaac |
| 107101 | ttaaaataaa ctaccattca acctagtcat cccattactc agtatggtgt atgcacgcca |
| 107161 | cacttttttg gtctaatcca tttttttttt tttgagacag agtttcactc tgttgtcctc |
| 107221 | tcactctgtt cacaagaaaa taaatcattc taccagaaag acacatgtac tctcacaatt |
| 107281 | tcattggaac actattcaca atagcaaagg catgggaatc aacctacgtg cccatcaaca |
| 107341 | atggattaca ggccaggcac agtggctcat gcctgtaatc ccagcacttt gggaggtcaa |
| 107401 | ggtgggcaga tacctgtggt cagcagttca agaccagcct ggccaacatg gtgaaactct |
| 107461 | gtctctacta aaaatacaaa aattagccgg gcgtcctggc acctgcctgt aatcccagct |
| 107521 | acttgggagg ctgaggcaga agaatcgctt gaacctggga ggtggaggca gcagtgagct |
| 107581 | gagatcatgt cactgcactt cagcctgaga aacagagtaa gattctgtct caaagaaaaa |
| 107641 | aaaatggatt agaccaaaaa agtgtggtat atatagacca tagaatacta tgtagccatt |
| 107701 | aaaaaaaaat gaaatcatgt cctttgcagc agcacggatg cagctaaagg ccattgtcct |
| 107761 | gagtgaatta atgcagaaac agaaaaccaa atactgcatg tttcacttat aaataggaac |
| 107821 | tatacaattt taactaacac tactaagtga tagaattgag aattgtacct ctgactccag |
| 107881 | actcagtcct taactattat agtgtttttt gtggggacca tatgagtgca gaggtgaaaa |
| 107941 | tactgacaga tattgaaaga tgctattggg taaataaact acaagagagt tcagagaaat |
| 108001 | taatgataaa aatgatcata ctactcctct cacttaccta acttttaagc aaacccattt |
| 108061 | gggaggtcct ttctaaaaag aaaataaaag agagaaaatg agaatgctca agtagatagt |
| 108121 | actattgaaa caggttacta aaaatgcatg tggtatcacc aaagaacctg ttctgacagc |
| 108181 | agagaaaatg aaattaggct ttctgtcctt atactcaagg ataactgtgc cttattgcaa |
| 108241 | atatttattc caactgttcc ataaaatatc tttgagtttt gaaagcaaaa aaagttaaaa |
| 108301 | ataattttct tacaggaaaa tagttgatta ttgaacttca ttaaaagttt ttaatgtcct |
| 108361 | ctctttgaaa gacactgtaa agaaaatgaa atgacaagcc tctggctggc agaaaatatt |
| 108421 | tataaaacat atgtctgata aagaacttat attcagaatg atatggtctg gctgtgtccc |
| 108481 | cacccaaatc tcatcttgaa ttgtagctcc cataattccc atgtgttatg ggagggactc |
| 108541 | agtgggagat aattgaatga tgggggtggt ttcccccata ctgttctcat ggtagtgaat |
| 108601 | aagtctcatg aaacttgatg tttttataag gggtttcccc tttcgcttgg ctgtcattct |
| 108661 | ctcttcactg ccaccatgta agacgtgact tttgtctctg ccattattgt gaggcctttc |
| 108721 | cagccatgtg gaactgtcag tcaattaaac ctctttttct ttataagtta cccagtcttg |
| 108781 | agtatgtctt tatcagcagc atgaaaatgg actaatacag agactatata aagaacattt |
| 108841 | acaactcagt aataaaaaca gaaagcaatg aatcaagcta agtagttggc aaaaggttta |
| 108901 | aacaaacatt tcataaaaag aggttgtatg aatgacccaa agcatatgaa aaaatcatca |
| 108961 | gcctcactag ccattaggaa aatgtgaatt aaaactatga tgagatgcca ttacattctg |
| 109021 | actagaatgg ccaaaataga aaacaaaaca gagtacctac caactgttgg ccagaatgtg |
| 109081 | aaataactga aacactcgta tattgctggc aatagtatga aatggtgcaa ccactctata |
| 109141 | gaactctggc agttttttct taagttaaac atacacttat catatgattt gacattttta |
| 109201 | ctcataggta tttactcaag agtgaggaaa ttgtatatcc agaagaagag aaatacatga |
| 109261 | atgttgatat cagttttatt tataatggcc aataattgaa aaccatttga atgttcatta |
| 109321 | acaggaaaga gaaatttttt aatgtggtat atccaacact actcaggaac actgctcagg |
| 109381 | aacaaactac tgattaatac aacaaaacag aagaacttca aaaacattat gctgagttaa |
| 109441 | agaagcctca catataacaa tacacactct ataactgtat gtgtgtgtgt gtgtgtgtat |
| 109501 | atatggcatc tatatatcta tatctataga tatatagatg cataaaattc tagaaatcaa |
| 109561 | aaactctctt attgttacca aaagcagata agtagttgct tgggaaaaag ggctggaggg |
| 109621 | gagagggtaa actgcaaagg gcactgacat tgtggtcaca caagtgtata cattgtcaaa |
| 109681 | actctaactg aaatgtggac taacatgttg cattttatta tatgtaaatt atacctcaat |
| 109741 | aatatttact ttaaaaggca taatgtaaaa tatgtatgta cacatacata caatataatt |
| 109801 | actaaaaaac cgttatcttt ttatgtggca gttactcata ttaaactttt taaccctctc |
| 109861 | aattctctca acacatcagt atagtaatag tggcaagtcc aaacttccct aattctattc |
| 109921 | caaataactg ttctaattat ctgtagctca ccatttaaat aggagtactt gagataataa |
| 109981 | aataattacc taaatcaccc atctcaatga atgatttaga attgaagagc cattgccaaa |
| 110041 | caaaagattg tagatttaca attaacatct aagtatgctg tagtatatgt ttttaattta |
| 110101 | caatagaatc tgccaataat ttcctcaaat aagtttcaaa ttcctaataa tgtagaaata |
| 110161 | catgtcataa atacataata ttcttaggta agatgctatt atagagaaga atttaaaaca |
| 110221 | tttacagtga ccttcacaat aaaagaaacc aggggaagca taaaatatct tttaaactcc |
| 110281 | ttgtagactc ctgtttctgt ctgcaatatt caataatctg tggtctgaat ggcttatgaa |
| 110341 | attgtgagtt tctcatcgtt tagcccatac cttagctgcc accaaaagaa aggcctgtct |
| 110401 | taacttagcg tttcctagaa tcagaagaaa ggagtggctt gaaggagaga agactccaat |
| 110461 | cgtctcacaa agcatgtaga tcactgtgtt ctgatacagc tcagaaatcc atgatatgag |
| 110521 | aacacataga aagaaactag catagaataa aaggaatgag atcacaattt tcaaggcatt |
| 110581 | tgtatggacc ttggtcctgg ggtctctgtg tcctttgtaa ttgagttgca ttttctggag |
| 110641 | atgtttctgc agggagaaaa ttaacaggag aaaagagatg aaggccacag taaatggtgt |
| 110701 | tagactgaac atagtcatag tgaatttgac cgacactgaa aatgtttcaa agtcactcat |
| 110761 | actgaaattc caagttgtgt ttctttcata tcggtccagc cagtctttta tatgcatgtt |
| 110821 | tatttgtatc agatttaaaa ataagaagac caaggttcct agcagtatca tcagaatcac |
| 110881 | tttgtttact ctccacttca aatagagaaa agcagggcta gagaaactcg ctattttgag |
| 110941 | caaataaaag atgctgaaga ttgtagcaag ccagagattg aagtgattag aaactatcca |
| 111001 | gctaaaaatc ataattctta atcctgttcc agacacaaat atggctagat aatgcagagc |
| 111061 | taaaaaccaa cttactaata tttcccagat cagcccaatt ctggagattg ccaagataat |
| 111121 | gaggagttta tcgactgagg acagctctct tttactgacc cagtcaatgc agttgatcag |
| 111181 | tactataaat ccattgctca aattcccaat tatgaattct gcaattatta caagagtgaa |
| 111241 | gatactcggc agggcacttt ccatgtcaga acagagaaag ttcaatgtct aatgtcactg |
| 111301 | ctggttattc actgatctaa aatgctattc acatccttga gtgtccagtg gagttcttct |
| 111361 | tccttctcct ttttctgctc cttctttcat tgttggctca acgtcaaagc agaaatctct |
| 111421 | aaagtttgct gatcgatctt cacataactg ttctggtgat atctttattt ttcttcaatt |
| 111481 | tctctgctga gccctagcta agatatttat gtcttcacca tgggcagaaa tatttcatag |
| 111541 | atgattatgc agcaaagtta aactcacatt tgcaaccatg caaataaaga tatattctct |
| 111601 | ttcattgttt tgtacttttt tgccttgtct gagcatagaa aattagattc aaccagcttg |
| 111661 | agttctgagg tacaaatatt ataaaaatct gattcataga atatgtagct aaatgaagct |
| 111721 | tttatggcta atagcatagc caatgaagct ttataaaata tgcaaagact tacctatgct |
| 111781 | atttcaaaag agtgctcaat ttcttgtgga gctaaagctg gatctggtca atactgtgac |
| 111841 | taaagagaaa ctcgtttaca aagcatccat ctttctcatt ccctgcctca tcactactta |
| 111901 | gcagtgctca ccaccatccc tccataggca ccaaatgcct tcacatttta tccgcttttc |
| 111961 | ccctcatatt tcttttttaa acttaaaagt gataaaaaaa caaattttac aaaatcagca |
| 112021 | acagctacca aagaaagcag tactatatta catcataact tgcaaaactg aaaaccaagc |
| 112081 | aaagaaatag atacactagt tccccttatc catgaggcct atgtcccaag acccccagtg |
| 112141 | gatgcctaaa acagcagata gtaccaaact ctatatacaa tatatttttt tcctgtattc |
| 112201 | acctacctat gataaagttt aatttgtaaa tcaagcacag taaaagatta caaaaataac |
| 112261 | taataataaa atagaacaat taaaacaata tactataata aaagttgcca ggggttgtgg |
| 112321 | ctcatccctg taatcctagc aatttgggag attgaggcgg gaggaccgct cgagcccaaa |
| 112381 | agtttgagac cggcctgggc aacataggga gaccctgtat ctaaaataaa taaaaattaa |
| 112441 | aaaaataaaa agtaaaaaaa tgtgttaaaa tatatagtgt ggtgtctctc actctctcaa |
| 112501 | gataccttac tgtactgtac acacctattt ttggactgtg gttgaccttg ggtcactgaa |
| 112561 | actgcagata agcggggact aatgtgttca ttgtgaagag tcaaggttcc tcaccccgcc |
| 112621 | ccactctttc aaaaactacc gaaaaaaagt ggattaccaa aattctgaaa acgtggagac |
| 112681 | tcaagaaaga ttgtctaatt caaaattaat ataaataaaa cactttatct tgattataag |
| 112741 | ataataagta gccatttagc tagatgaata caaagctatt tttatttaat aaagatataa |
| 112801 | tttgtgaatt atgaatgttt gtattttatt actcatgcaa tctttattgg gtgatctaca |
| 112861 | gagtactaaa aaatttacaa aataattgca tcatctttaa tgatttgcaa gtttcaatta |
| 112921 | taacttaaaa taggtagata ttttctattt tgttgttata atggtatatt tgtcaagata |
| 112981 | gaatagaaaa tgtctccttt aacagtttgt taggtttgta tatttatgac ctttaaatat |
| 113041 | gtagctatgt ggcatgtggt cttccatttg cattcttgac ccagggctga agagatcttt |
| 113101 | tatacttttt cattttaggg tgttcttcct acctatctag agaaaagggt ttggggatgc |
| 113161 | cggtcacaac ttgataatac cccaaatctg tacaaatagg cctgaggaat ggtactgtac |
| 113221 | ttgctggtat atgaaaaaat accacagttt ttagaacttg tgatataaca catgacaaag |
| 113281 | acatagagaa tggataacag gcttgcggtt ctgagctgca ttttcctggt gtgtttccat |
| 113341 | aagagaaaac atttaaaaat ataaatttaa tcaaagagat gtagtctctc acttctataa |
| 113401 | gagaaagaga atagatgcta gggagcatca cagaaaaact ttttcagcag tctgaactga |
| 113461 | aaaggaaaat gaggcaaagg gcaggaagag gaaacaatga aaggtttgag tatgtaaagt |
| 113521 | attttttaag tcactgatgg atgcagtttg ccaacgttgt gttaggatat ttgcagctat |
| 113581 | gtttttaagg aatattgaca tttaattttc atttctcata ttgtgcctat gagaatttgg |
| 113641 | tattaagatt tagctaacct catatcattt gtttagaaat atttcttttc tctttttcat |
| 113701 | ttttgctgcc tattattgtt acatataaca aatatagaac atttaatgaa gtttaaatgc |
| 113761 | tatagtataa agtgcacact aatttaaaag cctcccatgg caagaaagag aactttgtaa |
| 113821 | accccataac accttatttg ctcctttcta atcacaaccc ctatcttcat tccagaagta |
| 113881 | acaactatct gcaattcaaa tcacgttatt gtttaacttt gtatatttta atttgaatat |
| 113941 | gtatccctaa gcactagctt ggttttgcct ggttttggat tgcacatgaa tggaataata |
| 114001 | ctgtacacaa tatttttgaa tttaactatt tttgatcaat attatgtttc taaaattaat |
| 114061 | catgttgcat atagagatag atcatttatt caattgctgt atagtattaa tggaatgaca |
| 114121 | attcaaaatt tatctagtct agtggaaaca caaagttggt ttattttgag tttggagact |
| 114181 | atgatgatga tgatgctacg aacattcttt aacatgtaac ttattattga gtacatgagc |
| 114241 | atacatttct atgatcttga tctggaacca ttccgttgcc tatttcttct ggtcttgttt |
| 114301 | tggcagttac gactttctag atatttgtca gtttcaatta tttatgtttt catgtaattt |
| 114361 | tgattataat atcatcttat taaattttta ataccaacag aatatatcct ttaaattctt |
| 114421 | catattattt gcttttcttt tcatgattta tcttaccaga agtttgccta ttttgttagt |
| 114481 | ctattcaagt ctgtcttggt cgatcttttc agatgtatta tttttcatta atttcatctc |
| 114541 | ttatattgtt atttaattat ttttacttcc gttgagtatg tttgctgtta tattcctggc |
| 114601 | atacgttttc ctcttagaat ttttcagcat ttctcacaag gttttactat attttattat |
| 114661 | acttaatttt aaatattttc taaatttatt gtgatttttc ttccaccata gactattttt |
| 114721 | aaaatatatg ttcgagtttc catttacata aaattttctc tgctaaaatt tatgttatgc |
| 114781 | atatatggtc aattttgata aatgttttgt gtttgctgaa aataatgtgt atcctgtagc |
| 114841 | tttggggtgc agtgtttcat acatccacta gatctatttg taatcatgtt atttaaatcc |
| 114901 | tatttacctt ttctgagttt tgtcttttta ttctattatt tcttgagatt taatgttaaa |
| 114961 | ctatcctgct atgagtatag gtttatctat gtcttttaga agtcctgtca acttttactt |
| 115021 | tacatatttt gatgccatgc tgatgagtgc atagagcgtt agaattgtag tatcttcatg |
| 115081 | gcaaatataa cctctactca catgaggtga cattatctat tctaatgtct ttttccttaa |
| 115141 | aatctattta gcctcacgcc tgaaatccca gcactttggg aggccgaggc gggtggatca |
| 115201 | tgaggtcagg agatcgagac cagcatggct aacacatggt gaaaccaagt ctctactaaa |
| 115261 | actacaaaaa attagccggg catggtggca ggcacctgta gtcccagcta ctcgggaggc |
| 115321 | agaggcaaga gaatggcagg aacctgggag gtggagcttg cagtgagcca agatcacacc |
| 115381 | actgcactcc agtgtgggtg acaaagcgag actccgtctc aaaaaaaaaa aatctattta |
| 115441 | gtatatatta aaatgattac attagcttat ttttgttagt gttttatggt gtgatttttc |
| 115501 | taaattttac ttttaacttc tctttgttat taggatttag atatatatct tgtacatagc |
| 115561 | atagaggtcg taaaatggga cctagagcta tatgctaaca ttttattcct tgtaggataa |
| 115621 | gaaatgtagt cttgtaggag tcccctctcc cccttgaagt tgtatattat gtaaattttt |
| 115681 | ggtcaataaa atattgtgga aatgacatgt tgcttcagag tgagagcatt tactcaccac |
| 115741 | tataagactg caaatttctc atcttcttgc cttgacactc aaacaagggt caataaacca |
| 115801 | tctatcagcc tggattcttt tgtgaccact atgagcacag gacatgtaac agaaatcaaa |
| 115861 | aacaaaaaca aacaacaaaa acttctccta cgtggagtcc ctgagctttg gggatcttgt |
| 115921 | tagtacagca tagactaagc tatcttggca agtatgagac tggcctcttg aactggtatg |
| 115981 | ttactaaaac aaaacaccta acttatgtga cactggctta atacttgatt cattggcagt |
| 116041 | gagtggcaag aaaaaacatt actgtttact gtaaaacaag atagggagtg gcaaaacatt |
| 116101 | cgatagcagt cacctgtggt tatgtgggag gtaaatcaca tatttgaaag ttttgtaact |
| 116161 | ctagaaaaaa aatataattg aatatattaa tggttcattt gacaattaaa aaaattgact |
| 116221 | ttagaaaata attgcctact gtctagctaa atagcaaaca gagggagatg ctctaaaaga |
| 116281 | aaatgatatt tatttgggag taatattgca atgagaatac atgtgccata gtaaattatg |
| 116341 | tgtgtattca gggacattag aaaagacaag gatttctaaa agaggaagat tacataattg |
| 116401 | ttttgagata attaaccttg gatccaagga tcaggaacaa gggtgacacc tgtctaaggt |
| 116461 | tagacaagga ggttctgagc agatgtcctt gcagaagtat ctttttgtgt agggttgcag |
| 116521 | tggcctttgt gcaatgttgt tgttttgtag agtcttttct ggtagctctt gttataagtc |
| 116581 | gtatttacat taagaactct cccctcatgg ccttctccag ctccatttgt cagagtttta |
| 116641 | acacaagtga cttcattttg attctgacaa ttttatacta ctttgcaagc aggaaaaaaa |
| 116701 | tatatcagaa agaagagtct agaaacttga gattttgcag agctggaaga ggcaactgct |
| 116761 | tctcaacact gaatagtatt tgagtttcta agcctggtta aaactcagcc atggctgggc |
| 116821 | acggtggctc acgcctgtaa tcccagcact ttgggaggct aaggcaggtg catcacaagg |
| 116881 | tcaggagatc gagaccaccc tggctaacac ggtgaaaccc catctctact attaatacaa |
| 116941 | acaattagcc tggcatggtg gcgggcacct gtagtcccag ctacttggga ggctgaggca |
| 117001 | ggagaatggt gtgaacccag gaggcagagc ttgcagtgag ccaagatcat gccactgcac |
| 117061 | tccagcctgg gccacagagt gagactccgt ctcaaaaaaa caaaaaacaa acaaacaaaa |
| 117121 | aaactcagcc ttaagactag gattatctca agggtatggt tgttacatcc atgttaaaaa |
| 117181 | ttctgactag attatattta cagaaaaagg ttcaactgac agttactgct ctttcagttt |
| 117241 | gacaaggtaa tatcaaggca gagtgattaa atatctggtt tcctgaatga agccttacaa |
| 117301 | tgctcaaagt atctgatatt aaatccagag agaaaagcag aggggtgaaa aagcaacaaa |
| 117361 | acacagcaaa tataagaagc agcagactag acaaaaactg tgacttgttt gcttgtatgt |
| 117421 | gaagctgaca ggaattaagt agactaaaaa attgagtttc ttaaaaaatt gtgcttctga |
| 117481 | aagaaatgcc aacccaaact aggtcacacg tgtcaaagta tattaaaatg gaggccaggt |
| 117541 | ctgaagaatc cctaggcaga caaagccagt taggtctcat atgaccttaa aactgattga |
| 117601 | tttacaaaag taagcaaaaa ttaacttgag ctatttttta taaatgccta tagtaaagaa |
| 117661 | aaacagaact taaactcaac caatcaaaga cagccaacag gatagccaaa catttgattg |
| 117721 | gttagctata aagttacctt atttcatctg tcctgtcttt gcttttcttc tttgtctgtc |
| 117781 | ctataaaagc atccccatcg cgttccctcg gtagagctcc ggaaccacct cagtttggag |
| 117841 | ctttccaatt tatgaaccat catttgcaaa taaactttta aaaaacttta gtgtgcctta |
| 117901 | gttgacctta ttaacacatg ttgagctaaa accctgtatt agtctgttct catgttgcta |
| 117961 | ataaagacat acacaagact aagtaattta taaaggaaag aggtttaatg aactcacagt |
| 118021 | tctacatggc tgaggaggcc tcacaatcat ggtggaagac aaagaaagag gaaagggaca |
| 118081 | tcttacgtgg cagcagacaa gagagagttt gcaaggcaac tcccctttat aaaaccatca |
| 118141 | gatctcatga gaattactta ttcattatca taagaacaac atgggaaaga cccgcccacg |
| 118201 | attcagttgc ctcccactga gttcctcccc atgacacatg ggaattactc gggagccaca |
| 118261 | attcaagatg agatttggat ggggacacag aaaaaccata tcaaaccctg ttccagagat |
| 118321 | gcagtttaac atattatgag acaaatggga gctcacagac aatatgagaa tctcttgaag |
| 118381 | attacacttt ttttcctagt tctaaaatta catctttttt aatgagactt atattcttcc |
| 118441 | cccaactcag aacatttcct tgtctttaag ggagacctgc cacagataga gaaccataga |
| 118501 | aaataacagt aacaaccaca taaactgtca tgttagctat gaaaagagtt ggtggaagga |
| 118561 | atgggctctc accaagacag tgaaaaatag aggtaggtgg taaacaaata tgcaaaagga |
| 118621 | ccattcagaa acccaagcaa actatcattc tccccatttt actggtgcta taaatacctg |
| 118681 | aaaaaaagtg ccacagaaat ggattagtgt tatcatcaac aaatatattt gagccgggca |
| 118741 | cagtggctca cacctctaat cccagcattt tgggaggcca aaatgggaag atatcttgag |
| 118801 | cccaggagtt tgagaccagc ctgaacaaca taaaaagacc acctctctat aaaataagat |
| 118861 | aaaataaaat aaaataaaat aaaataaaaa aaatagctgg gcatggtggt gcacatctgt |
| 118921 | agtcccagct acttagaagg ctgagatgga aggattgctt gagcccggaa ggcccaggct |
| 118981 | gcagtgagcc atgcttatgc cactgccagt cagcctgggt gacagtgaga ctcatatata |
| 119041 | tgtatacatt taaatatatg tatacatggg tgagactcat atatacagat atgtatatat |
| 119101 | ttgtacgtat gagataagct ggtgagaatg aggtcattga agcagatatc ctgttccttg |
| 119161 | ccaattaagt cagtgatgaa ttccatagtt aataccacca tgaaaatgtc ctcctctata |
| 119221 | cctggcttct cttcagagag aaaaattcct aattttataa ttttttttat tttcaactaa |
| 119281 | gggattaata caataattaa gggattaaat atttaatatt tattgtttca gtacttctct |
| 119341 | cttgctatgt ccattaattc cattgctcat tgttaatttg ttgttaatta cattaaaatc |
| 119401 | agttcttctg taggatcatg tcatattgat ctttatgaaa tcagagcaat catttttcaa |
| 119461 | atgattcagc tggcaatttt catactaaca aatattttag ttttttataa gagccacagg |
| 119521 | tgataacttt ccatattcaa aagaataccc agtgaagtct agtgttctca gatgcagtta |
| 119581 | cggacagttt tcctttcact gttttcactt gttgcttcac cctaaacttc aatttatgta |
| 119641 | cacattacag tccccaatta ggagactaac aggatgttga agaatggcat tggcaggaca |
| 119701 | tttctgaatt cagtagcagg aatgaactca aaaccagcag tactgagtca gttcttgttc |
| 119761 | tcttctgact gaattcgtgt tcatggaaat gtttctgcaa gactgagtgg tcacagtgtc |
| 119821 | agaaggctcc tcaggttttg cctcttcaca catgtccatt tcccctttga ccttctgcca |
| 119881 | tgtcaagata cagctggaaa gccctcacca gaagctgagc agatatcagt gcctgctttt |
| 119941 | ggaacttccc agcctgcaga aacattaggt ctttatagat tgtccagctt caatattatg |
| 120001 | cagccttaaa aaggaacaag atcatgtcct ttgcagggac atggatgaag ctggaagcca |
| 120061 | ttatcctcag caaactaatg caggaacaga aaaccaaaca ccacgtgttc tcgcttataa |
| 120121 | ttgggaggtg aacaatgaga tcacatggac acagggaggg gaacaacact tagcgggcat |
| 120181 | gttggggcag gacgggggtg ggagagcatt agagaaaaga gttgatgcat gctgggctta |
| 120241 | atatctaggt gataggttga taggtgcagc aaaccaccat ggcacacatt aacctatgta |
| 120301 | acaaacctgc atatcctgca catgtacccc agaacttaat aaataaataa ataatacatt |
| 120361 | tttaaaagat ccacatgatc aaaaatgcaa aaaaaaataa attatccaac ttcaggtatt |
| 120421 | cttttatagc aacataaaat ggactaagac aaaactctgt ggtgaaatct ccaattattt |
| 120481 | gtaagatgaa gtcgtagaaa tgggtgaagt ccaaaaagca ggttataatc tatataaaca |
| 120541 | tttaagactt ttattaatat tgacaaagtc ccctacaaaa tgcatgcacc aatgtaagct |
| 120601 | ctcactagca ttaaacgaga ctgctcgtaa ctttgcacac attataattc tatgtaacaa |
| 120661 | tttacaaaag caaaatgtgg ccaatttgaa atgcaaaata gagcacttgt tattcatttt |
| 120721 | accatagtac tatactaaac ttacgttttt gtttaaatgt gaggctgaat tttttatata |
| 120781 | tgataatctg gtttttaaaa ttatttttat cttttttata aaacagtaaa agtattaaat |
| 120841 | ttttttctac tgtttatatt gcaaatagac tttaaagttt ttaatctatt atttaccttt |
| 120901 | ctatatttat tttcttgagg cattcagaaa ttcttccagt catgctatta aatgcattca |
| 120961 | ttttaaatgt actctgcctt taatatcatg tttaaacatg tcttcccaac tttcaagatt |
| 121021 | acaaaacaat tcttggtatt ttctctgttt gattgtttac tttttcaatt ctatatttaa |
| 121081 | tcctcttatt tactttggaa gaagaagtat gttacacatt tgtcttattt caaattgcaa |
| 121141 | tattgttgtt acaatactaa ttattgcagg gttaattttt cttcacatat ttaagatgga |
| 121201 | aaaatctatc tcacacaata tatttgtaaa caaaaaaaga tatttataaa ctctataatt |
| 121261 | tttttgttgc actggagttt cctcattctt tcatgatgtt attctatgcc actctttaaa |
| 121321 | gagaagtgtt ggaggacatg aaaggtgaga ggatagacat gtaactattt gttggacacc |
| 121381 | taacttagga tggacagatt atattatttt tatcagcaaa tgtgaactca tcagtctttt |
| 121441 | ccttaatgac atgtgcttcc tcaaattaaa ggcagtagag tcttattttg attttcttgt |
| 121501 | aaaagtttta aagaattgtt ttcaaatcta aagtgatagt acatctaatg tagtttttct |
| 121561 | gtttgttgtg agttagaaat acaattttgt cccaatatta aatgacagag gtctcaatat |
| 121621 | cattagttga acagtctacg tttttttttt gttgatttct aatgccaaat ctatcacgta |
| 121681 | cggaggttcc tatgtgataa gaataaacaa tctggtctca ccagacctgt ctttagtttg |
| 121741 | atctgctgac ctattactta ttatatacat tttatttctg tgtcacaacc ttcattgttt |
| 121801 | tgattttttt aataattttt ttgagacacg gtcttgctct gtctcaaaaa atattaaaaa |
| 121861 | aattttttta atcttttttt tttttttcct gacagagtct tgctctgtag cccaggctgg |
| 121921 | agtggctcaa ttaaggctca cttcagcctt gacctcctgg gctcaaacaa tcctcctgtt |
| 121981 | tgattctctg tgtagctaag actacagttg ccactatacc cagctacttt tttgatgctt |
| 122041 | ttagagacaa ggtctcactg tgttgctcaa gtgggtcttg aatgccttaa ctcaagtgat |
| 122101 | cttcccatct cagcctccca acatgctggg attacaagta tgagccacta aataaataat |
| 122161 | ttaaataagt cttgctatct actggatacc cctaccttaa ttttcttttt caagaattgt |
| 122221 | ctgaattatt tttgaatact taattcatgt gtaatttagg actgttttgt caatttgaaa |
| 122281 | ataaatttct ttaagattct ggttagaatt atattgaata tgtgaattaa tttgagcata |
| 122341 | atcaatgtct ttgaaaatta atttttgcat atctatatag ttcaaatcct tcactaaagt |
| 122401 | attatagtta tgtaatttca caatatataa catcatgaaa cacttttctg agacttagtc |
| 122461 | ataatcaaaa tatagttctt atttgtaata agaaagggaa tttttttaat tccacatttt |
| 122521 | aatggaccag tgctggtgaa taggtgtaat tttagcccat atatattgat attgtactca |
| 122581 | gcaagcttat caaatttgtg ttatttcaaa agttttcctg ttgattttta aattatctag |
| 122641 | gtggaattac tactattagt atataatttc tttaaatttc catccaaatt tctatgtaac |
| 122701 | aatatttttg tcaaagtgta ttagttaaat gacctaacga aattgtcaat aaacaagtat |
| 122761 | ctttgtctta atcttgatta aatgaaaagc tctatgttac actaataact atgcttctgt |
| 122821 | atttgtttag tagacataca agttattaaa tgttgcttct atttaaactt ggtttgggta |
| 122881 | tttatcttga ttaggagtta aattagatta aacatttttg caacatctgt tgagataatt |
| 122941 | ttatgtaaat taaaattatt tactttctag ttttaaatta tcttcaaatt cttagaatga |
| 123001 | atcttttcat ttgtaatata ttttaagtat atattatgag actaatttac ttttacactt |
| 123061 | ttttttagca gttttgtgcc tgtatctaat tgcctacctt ctcattattt gctgtcctct |
| 123121 | actgttttta ctcactttga gaataatttt ttaacttctt aaaataaatg agaataaaac |
| 123181 | atttaggttc actgaagtag tttggagaag gtaaaaatta tgacattttt aaacttagta |
| 123241 | aaagtttttt ataaatccat ctgaatttga tatcttgtgt atattttgat agtccattca |
| 123301 | aaatctttga aattgttgat ttattaaagt tttttactac taatataaat ttttgttaac |
| 123361 | ctatgctttt taaaaacttg gcaatttcaa ctgagttttt aaattttgga ataaaattat |
| 123421 | ttagaacttt gattatataa agctctgcta tttagtattt tctacatttc atcaattttt |
| 123481 | ctgcttctcg tatttcttga tcagtaccat cagacttttg aatttcctaa tctttaaaag |
| 123541 | attttggttg tactaccatt gtctattata ttttgatttt ctatttattt attcattcat |
| 123601 | ttatttattt actactttga ttttggttga catccaaaag cataattact gcccttcaga |
| 123661 | caaattctaa cttttccttt tgctgagtct atgacttggg aaatttactt aaccttgcta |
| 123721 | tcccttggat ttttgtttgt ttagtttttg agacaaggtc tcacgctgtc acccggggct |
| 123781 | ggagtgcagt ggtgcgatct tggctcactg tagttttgac ctcgcaggct caagtgatcc |
| 123841 | ttccacctca gcctccctag tagctgggac tacaaaggtg caccaccata cccggctaat |
| 123901 | ttttgtattg tttgtagagg gcagtggggt ctcaccatat tgcccaggct tgtctcgaac |
| 123961 | tcctgaactc aagcatccac ctgtcttgac ctcccaatgt tcggggatta caggcaggtg |
| 124021 | ccacagcacc ctgtcatgcc tctgattctt taaaaaccaa agggaatgag actgttttta |
| 124081 | ggaataaata taaaataaat agcacaccta aaccacctag aacagtacca tgtactaagt |
| 124141 | aagcgcaagg tacatattag ctccttctta acttattcac ccttctttaa atttttctgt |
| 124201 | tgttcatttt tcttaactga aggtgaatat gtgcctcatt catctggaaa acttttttat |
| 124261 | aaaacatgta ctaatggata tggattctta gctttagccg gttttttcat tttgattgtg |
| 124321 | aaaagctttt tcgttgctga gttctaaaca tttatctttt atgtagaaat gttcatcttt |
| 124381 | ttctcagtta tagagaatag ttcaattttc aaactttttt gattgattta acattttact |
| 124441 | actttacatt ctacgtaata cttattccat ggaatctgtt ctttgttata gtgtacatgg |
| 124501 | tttaggtgtg taaatgtttc atgtgtattt gaatgtttgt gtccagattt ctgtcaagta |
| 124561 | cagatttcta cataaaagta ttgaattaat ttttgctaat tgcattctta aaatattcca |
| 124621 | tatcattctt tcttctgctt gatatggcag tttattaagt gtgtcttaca ttcttattat |
| 124681 | ttgtggattc ttcagtttct gtttgtgagg cttctgcttt atctatttac gtaatgcatt |
| 124741 | gttaggtatg tccaaattca tgattattgc atcttttcca tgaatttttt ctttttcttt |
| 124801 | tttttgagac ggagtctcgc tctgtcgccc aggccggact gcggactgca gtggcgcaat |
| 124861 | ctcggctcac tgcaagctcc gcttcccggg ttcacgccat tctcctgcct cagcctcccg |
| 124921 | agtagctggg actacaggcg cccgccaccg cgcccggcta attttttgta tttttagtag |
| 124981 | aaacggggtt tcaccttgtt agccaggatg gtctcgatct cctgacctca tgatccaccc |
| 125041 | gcctcggcct cccaaagtgc tgggattaca ggcgtgagcc accgcgcccg gccctttttt |
| 125101 | ttttttttga gacagagttt tgctcttgtt gcccaggctg gagttcaatg gtgtgatctt |
| 125161 | ggctcagtgc cacctctgcc tcccaggttc aagtaattct ccttcctcag tctcctgagt |
| 125221 | agctgggatt acaggcacac gccaccatgc acagctaatt tttttgtatt tttagtagag |
| 125281 | atgggggttt caccacattg gccaggctgg tcttgaactc ctgacctcag gtgatctgcc |
| 125341 | cgccttggcc tcccaaagtg ctggaattac aggcgtgagc caacatgccc agccttcgat |
| 125401 | gaattgtttt attcttacta ctctgtaatc acctgtcttt aactgttact gttgtttact |
| 125461 | ttaaatgtta tgtgattatt agttataatt cagttaaata tgacaaatat aaacaagcaa |
| 125521 | ttgaatatat gcatgtggaa gagcatctat ctttcacata actaaaaaat acataggaaa |
| 125581 | gtttttgtgg cacaaattgt ccaagggaat agtgtgattc tatcaccaac aaatgatagg |
| 125641 | ataatagaca ttaggcataa gtagcagtta ctgtgctcat tagttagtgg tagattctac |
| 125701 | ccatttattt tctgtcactg tggttttctg ctttaggtat gcctcttgaa agcagaacat |
| 125761 | agtcgtgttt tgttttctta tttattctct gtcttttgat agatcaagca aatttattta |
| 125821 | aattcatgaa catttctgat aacattgaac ttgcttttac tgtcttatta aaattttgct |
| 125881 | agttatcact ctcttttcac tctttttttg ctttgagatt aaagtgataa gttcttttta |
| 125941 | ttcatttctc catctgtttt tttttttttt tttttttttg agacggagtc tcgctctgtc |
| 126001 | gcccaggctg gagtgcgatc tcagctcacg gcaagctccg cctcctgggt tcacgccatt |
| 126061 | ctcctgcctc agcctcccga gtagctggga ctacaggcgc ctgccaccac gcccagctaa |
| 126121 | ttttttgtat ttttaataga gacggggttt caccgtgtta gccaggatgg tctccatctc |
| 126181 | ctgacctcat gatccacccg cctcggcctc ccaaagtgct gggattacag gcgtgagaca |
| 126241 | cggagcccag cctcatttct ccatctattc taagtttcta tttttatata acaaatcatt |
| 126301 | ccccaaattg caggcttaaa acagcagttt attacttttt tcctcacgat tctgtgaatt |
| 126361 | gcctggactt agtggatagt ctctcattgt ggtatttcat gtagttgcca tcagataagg |
| 126421 | gatgggacca cagtcatctg aaggctcaaa tgggatgaac atctacaatg atccaactca |
| 126481 | atggctgaca tagggaggat tccctctttt tctactgatt ggaatagttt cagaaggaat |
| 126541 | ggtaccagct cctccttgta actctggtag aattcggctg tgattccgtc tggtcctgga |
| 126601 | ctttttttgg ttggtaggct attaattatt gcctcaattt cagagcctgt tagtggtata |
| 126661 | ctcagggatt caaattcttc ctggtttagt cttgggaggg tgtatgtgtc gaggaatgta |
| 126721 | tccatttctt ctagattttc tagtttattt gcatagaggt gtttatagtt ttctctgatg |
| 126781 | gtagtttgta tttctgtgga atcagtggtg atatcccctt tatcattttt tggggggtct |
| 126841 | atttgatttt tctctctttt cttctttatt agtcttgcta ctggtctatc aattttgttg |
| 126901 | atcttttcaa aaaaaccaac tcctggattc atggatttct tgaagggtgt tttgtgtctc |
| 126961 | tatctccttc aattctgctc tgatcttagt tgtttcttgc tgtctgctag cttttgaatg |
| 127021 | tgttggagag cagtttctca taaaagcttg tggattcaac tgtaaaagtg gcaatccact |
| 127081 | ctgactcccc tctccactgc agagagcttt cttctttcac ttattaaact tttgctccaa |
| 127141 | cctcaccctt ttgtgtccac actccttaat tttcttggtc atgagaccat gagctcagat |
| 127201 | aacaccttag taataatatc aatgacccgg acctgttttg gtaacaccag ctctggggcc |
| 127261 | aggacacagg ggcactctca ggcagaagac tgaaatctgt gcaagtggta tacaaattta |
| 127321 | tcccttcata cacaaatctc aggccagtct ctctcttgga cttctaaaat tttacattca |
| 127381 | ttgtgatgag aatcaacatt tttggttcat ttcaaaggac aggctcaagt tttgccaatg |
| 127441 | ctaatatggc tcagcagaaa aatgcatgtg gcttgtatta agaaagcaca taaaggatct |
| 127501 | acattgactg ggttttcata atcacaaaaa acgaataaat ttgtatgagt atctttatat |
| 127561 | gaaggcaagg aggaaaacag gtgaatggaa gcctacgtaa atgtgcttat gatttaaaac |
| 127621 | aaacttattt cctcaggaaa aatatatttt tcacctcacc tgttaaagtc ttaggaaagc |
| 127681 | ttaaccttga taatctccta ataacagaac ttatgtttct tttctgaacc tcattccttc |
| 127741 | tcatattgta ataatgaaat gaataaatga atagcaagtg agtagtggtg cacactaaac |
| 127801 | tctgagtact gactgccttc tattccactt aataccttta taagaacatt gttatgtacc |
| 127861 | taagtcatgt catctataaa actaaggtaa tgataacaat catatgtccc taatataatg |
| 127921 | gttttgatga ttaagtggaa catagtaaat gtttaaaaat gttttctatt ataaccaatt |
| 127981 | tggctaggat aggtggaaag aatagaaata tgataattaa ttgtgaaatg tataatatat |
| 128041 | attaataggt catcaacatc aaattaaaaa atatagatga ttaccatgat agtgaaacca |
| 128101 | aagtttttag acaagtcaaa gcaaagttga agtctcagag accattgtgt ttatttgcta |
| 128161 | actgtattat cagactgctt agttgataca gtgaatacaa taattcttcc aatgtacata |
| 128221 | aactgccaga caagcaactc aatgcaattc actcactctc ggagaagttt atcccctctg |
| 128281 | ccaatgttca tctttcctgg caaaccatcc ttcagtattc caggcaaact agagcaaggt |
| 128341 | gaagttgaag aagatcctac tttatattcc attcagagag cttccgggtc ttcaaaaaag |
| 128401 | atttagtcat gtgggtgtga aagagctgag tcatgaaatt tggaccttat tgtaagtcca |
| 128461 | gaaggaaaaa caaggggttt gaagcacaag ggtgatatga tctaatttct acttttaaat |
| 128521 | gatcactctg attgtcgtga gggaactgat atgggatgta ctgggggaag aatgttacca |
| 128581 | ggaccaggtc gaggctgtta aagacctcca ggagagagca ttgtgggccg aacaacaagc |
| 128641 | ttctagtaaa gatcatgcaa gtggacacat ttgagatgta ttttgaaggt ggaagaaatt |
| 128701 | agataagcta atgggtagtg cctggaggga gtgaggattt ttgaaccact aggtaaacac |
| 128761 | ctgtgagtaa cagagaaaaa gttgggatgt gttatgaaag atgaatccta aaatttcata |
| 128821 | tgttgaagac tgatgcttca ggacctcaga atgtgaacat ccatagtaga agggaattgt |
| 128881 | gttgttgacg gctgccaccc actctaggtg cccaagctcc tgcagcagtc ctacttctcc |
| 128941 | ctttcaccca cactcactcc cagacagtgt ccctttcagc tcttccatca ccccttcttt |
| 129001 | tgatttggct ggtgagagca tagctggtat ttatactttc ttgatatgct agtctctttt |
| 129061 | gctgattgtt gacattggag aaggtcatgg aagggacctg agctgagcga cctttaggag |
| 129121 | catttcctgt tcttttcctg tactctgctg ccaaggagaa atctgtcttt caccaggctt |
| 129181 | gcagctttct ccgttaccca catcatcagg caatatctgt ctggcacccc tctttgttcc |
| 129241 | ctttctctgg ccttcctttt tgacatgatg gttgacctga aatcaccaag gcaggactgc |
| 129301 | tcatctccag gtctaatgga aaacagtatc tggtgaaacc aaagaaaata atcccagtaa |
| 129361 | cccactggga gctgcttggc ttaagcacct accctgtgcc taagatacaa cccatcaagc |
| 129421 | ccagctaaaa ctgatccagt ttccacaggg ccagagaaga attgccaatt aagaatgcat |
| 129481 | tcccacttca aatatgtcta tatttatttc agggaaaata tcagtctgcc tgggtccctt |
| 129541 | atgagaacat tgttgtctgt agaaaaatct aatctattag gaagccttct tgttttgtta |
| 129601 | tctgcaactc ttcagaaggg ccagaccaga ccctcgtatg cacagtacag gatccaccca |
| 129661 | tgcacatctg tctagggcct tgtctttatg attttctgtc acatggcctc tcagccatgc |
| 129721 | ctccaaccaa gctatcctca aataaacact agccgtgcac aaaaagtgaa aaattacaca |
| 129781 | aggcctacac aaggtctgaa atggaaagtg cagctggtga taccgagtag tatattttat |
| 129841 | gaattgagga acagggaatt tctaggtatc acagagcaat cagcagctga gaagaggagt |
| 129901 | accaatccag gaccctggaa ggatttttca gaaacttaat agtttattgt ccaattgtag |
| 129961 | agacaaaaat gaaaataaag atagtgagga gtaccataca ccattgctta gaagattaaa |
| 130021 | taaatgtcat ctcccacaag tcatgtgggg agaaaaaaac tatataggaa gaaaattaca |
| 130081 | aacgacaagg tagtctcagc atataaaata tcaacaaatg aatgtaagaa agagaatgag |
| 130141 | aaagaattag tattagaata tcatagaaaa aatcatcttt gtgaaagaaa actctgatga |
| 130201 | aactggtgac ataaatactg agtaaaaaac agaagaccaa tcaaggcatt tgaggcttga |
| 130261 | acaggcatta caaatcatca agttgaatat ataagtatgg ccacttagtt attagtggtg |
| 130321 | aagactacgg tatttgttag aagaaataaa gtagatgatg aattctatag aatattctgg |
| 130381 | agaaagtgga ggtcttagct acatgacatc gtgtggctca ctttgggaac catgtttcag |
| 130441 | tttgtttagg tctagaaatt ccttggtggt ttctgtgttc agttttgctt taataatccc |
| 130501 | taaagatatt ttttaaaata caaatttttt agctttcctg tattgtacag caccaacgct |
| 130561 | tgcctgagtg tagtgagatt cacagagttt ggaaatttcc tgtagcatga ggaggctgcg |
| 130621 | gggcagcctt ctgtcatcta tgtgtaccca gcagtgtgct gtcctcacac aactcacgga |
| 130681 | taagcagaaa gcatctctat tacatgaaaa atttagctaa atatacaaat atccatgcac |
| 130741 | acacatcatg agagatggaa ataggctatc aaaagagtca gacatattga ccgtcttcac |
| 130801 | tgttagctgt aggattgctg cccaaccccc tgacacaccc acacaaactc acatacacaS |
| 130861 | aaatgaaaag cacagtggaa agcagaatag cagtctttcc aaacagattg aaaaacaaat |
| 130921 | gatgtctaaa gttcaatcct gacactgtgc tgattgcttg gacacagttc cgtcaaaaca |
| 130981 | tttgtccaag tcatagttgt accttttaga aattagccac aaacaacatc ctccccaacc |
| 131041 | cttcctgcta ggctagagtc ccagaagaaa tgagggatac acttgacctg aagtaagcaa |
| 131101 | agcagaaccc agtctctgag gcgaggaggc ccacctggta gggagctcaa atgcgccatt |
| 131161 | gtcctgcttg tctttataaa gggagctgac acgtttctcc cagcataaag ttgggagtga |
| 131221 | caccagagcc ttctgcaaga tgcttctgat tctgctgtca gtggccctgc tggccttcag |
| 131281 | ctcagctcag gacttagatg aaggtaagcc gaattggggg aagatattgt gactctgatt |
| 131341 | ggggtttacg ggcgaatgct atagaggggg aaagtggagg gaagagagga ggatgagaaa |
| 131401 | acagatagga ctgaagagtt ctcatgccaa ggatcagaag acctgttgtg ccttcattcc |
| 131461 | tcatcaagac ctcataattt attgattgca caaatagaac ccaataaaga atttgtaccg |
| 131521 | ggggagtgag agagtgagat ttgcatttat agagacatgg gactgctggg aaggatatgg |
| 131581 | agaatgcaag acagattcag ggaagtgcag ctgtgaagat cctatactga tcccagtaga |
| 131641 | cagggatgat ggtggccttg ctggacagtg gatgagtatc catgaaggag ataaacacat |
| 131701 | gtcagagcta ttgctgaggc agagaattgg gtaaacactt gcctctgtct acatagagtt |
| 131761 | agagaatcac cagagtgaaa tattgtcatt tttttctctc ctgcatgtag tattttaatg |
| 131821 | tgctgggacg ggcatttgta agattgtatc taagtggcta tgtctggtgg ctcctgttga |
| 131881 | gaaagcttgc aaacataaac aacatattta cagatgaaag agggcagaag gatccccaaa |
| 131941 | tatttcattg aaatactcaa gagcccttta actaaataag cactaaggct taaggaatca |
| 132001 | tgagaggaca aacaggggcc cttctatgtt gagttcctgg ttgacgctca gtgtagtaac |
| 132061 | aatcctgctt tcccttacat cttcttccac ttccggtagc atcagagagt ggctgatgag |
| 132121 | atctcaaagg ggatgcacag ggtgtgatca gaggtccttt atcctcgtag aacactatga |
| 132181 | gctctgaatg attcatgcag taacttttcc catcatcctg tacttctttt ctagatgtca |
| 132241 | gccaagaaga cgttcccttg gtaatatcag gtaaatccca ataaattctc agtaaactct |
| 132301 | gtctccattt ttccctgaaa aattgatcag ttctccagtg tcttcttatc atccttgtca |
| 132361 | ggaattggct aatatcagtg ccccagagat ataaacagtt ttctcccaac cttgattctg |
| 132421 | gggaccatga gtaaagaaat ttgatttttc accaccctaa tgtggattaa gaggagttct |
| 132481 | aattaggaag ccttgggaag gggggaggtt gggagttgag aggcaggtca gggagagagg |
| 132541 | ggccggccgt gtggtgaaga cagagagata tgaagacagg agggttttcc agcatgagct |
| 132601 | cagctcttct tgtttcaact cacacagatg gaggagactc tgagcagttc atagatgagg |
| 132661 | agcgtcaggg accacctttg ggaggacagc aatctcaacc ctctgctggt gatgggaacc |
| 132721 | agaatgatgg ccctcagcag ggaccacccc aacaaggagg ccagcagcaa caaggtccac |
| 132781 | cacctcctca gggaaagcca caaggaccac cccaRcaggg aggccatccc cctcctcctc |
| 132841 | aaggaaggcc acaaggacca ccccaacagg gaggccatcc ccgtcctcct cgaggaaggc |
| 132901 | cacaaggacc accccaacag ggaggccatc agcaaggtcc tcccccacct cctcctggaa |
| 132961 | agccccaggg accacctccc caagggggcc gcccacaagg acctccacag gggcagtctc |
| 133021 | ctcagtaatc taggattcaa tgataggtat gattccagtt tattatccat caaaggctcc |
| 133081 | aactgctaca gttctccaac ttcattgtgc cagtgaatct attgaaaagc tgttaatatt |
| 133141 | tccgtgtcct ggaacacatt tctcatgagt tttgttcaaa tattctggga taaggtagca |
| 133201 | agatcttgtt tttaaacaat ctcttgaagg caattccaat tttgagaatc actatcttca |
| 133261 | aattacctct cttaaatagg gttgggaatg aggacataga atcatgttct ccctttggca |
| 133321 | ctctgtttcc tttcctcaaa ctcaaagact cccatttatt taaagtttta cctgaacact |
| 133381 | ccttgttcag gacaggctca gtcctgcctc acactagcat ttcaagtcca gtattcctgc |
| 133441 | taaatggtcc ttggactttt agctgttaaa tggtatctca ttttttaaaa cacatttcac |
| 133501 | atttaaagtc atacatgctt aagctaacaa aaactaatct cactgaatag acatgtacca |
| 133561 | agctaaatag caattcattt ctcctcccct ctacccttac caaaactccc actttttact |
| 133621 | gtttggaaac tcctctttga aatatttatt gctacataac tatatataat tttcccacca |
| 133681 | ctaataccac actttatatt cagttctgtg tcttacttat tggaaaaatt tatttcttag |
| 133741 | ctttcacatg agtttattta gatctcttca gtgtttaYtg gttagttttt ttttttacaa |
| 133801 | ttatatatga tgctattgtt tggtttttcc ataatttact taaccaatcc ttgtcactga |
| 133861 | acattaaggc tgctttcaca ctgtccctat tacaggatat gttgcagtcc ccatctttct |
| 133921 | gaaaataacc cttaacgtat ccaacagcca caaagcatga acaacctaac aataatcgtt |
| 133981 | tttcgtcctc atctaagcaa |

Following is a genomic nucleotide sequence of a GRID2 region (SEQ ID NO: 3).

| >4:94744101-94841750 | |
|---|---|
| 1 | tcaactcctg ggctcaagca atcctcccac ctaagactct aaaataactg ggactaccac |
| 61 | ccagtcaaat gccaccacat ttgactaatt tttataaatg ttttgtagag atagggtttt |
| 121 | gatatgttgc ccaaactagt cttgaatttg gggcctcgag tgatcctccc acctcaacct |
| 181 | cccaaagcca tgggattcca ggtgtRagcc actgtaccta accctcttct ggaaaatctt |
| 241 | aaRgccttag attgcattcc tgtctttata ggtcatattt ctcattattc tcttcttttt |
| 301 | gaccttttct ttgcaacttg agagttttaa aagttcactt attgtgtaac ttattcatta |
| 361 | gtcctgatat atcatttgca ttctttacta ctcccaactg ttatttttat tctgcagtag |
| 421 | aagtttgtct ttattctttt ttgttttctt ttctcatatt atctcatctt ttatatcatt |
| 481 | accttccttt cagcctattc tctttgttgg ttctctactg atgtttcacc tagattttca |
| 541 | ttgtttaaag gcattcatca gattatattg caacattttt attgaatgca gccagaattt |
| 601 | atgtgaggta tgtactacct ctgagacttg agggtgatat tctgatttgt ttagggtgca |
| 661 | atgagttttt gtgtttgttt atgtttctat aaaccatcca ggcccaatct ttatttttgt |
| 721 | caatagttaa agaaagtgaa gcatcttttg cctcctctac aatccattta gttaagttca |
| 781 | gtttttcttt ctcagaccta gagttaaaga tcaaatagat atgttcagca tttaatcaaa |
| 841 | tttaccatgc atagtaggta ttccatgtat aggtttttga cagagattta ctaaagttat |
| 901 | ttggtctaat tatgacagag catttattcc agacactgtc ttgtttatac tacttatcac |
| 961 | tcagtgacta aatcttccaa aacaaaaatt attgtcatct caccctctct acttccataa |
| 1021 | ccttacggtg gtttctgctt tgagtagtag tctctggata tttgtgaaag agtgtaactg |
| 1081 | agaaggcagc aggtagtgaa taagagaaca aagtctctgg atactaataa agatagttct |
| 1141 | catatagttc aggatagctt gctgcttact tggccataaa gctcttacag cctaaagtta |
| 1201 | ggcataatca aaggaaatat tgttgcggga agtcagggac cccaaatgga gggactggct |
| 1261 | gaagccatgg cagaagaaca tggattgtga agatttcatg gacatttatt agttccccaa |
| 1321 | attaatactt ttataatttc ttatgcctgt ctttactgca atctctaaac ataaattgta |
| 1381 | aagatttcat ggacactaac cacttcccca atcaataccc ttgtgatttc ctatgcctgt |
| 1441 | ctttacttta atctcttaat cctgtcagct gaggaggatg tatattgcct caggaccctg |
| 1501 | taataattgc attaactgca caaactgtac agcatgtgtg tttgagcaat atgaaatgtg |
| 1561 | ggcaccttaa aaaaagaaca ggataacagc aattgttcag ggaataagag agataacctt |
| 1621 | aaactctgac tgcctgtgag ccgggtggaa cagagccata tttctcttct ttgaaaagca |
| 1681 | aatgggagaa ataatgctga attctttttc tcagcatgga acatccctga gaaagagaat |
| 1741 | gcgcacctgg ggataggtct ctaaactggc acccctgggc atggtcatct cttatggtcg |
| 1801 | agactgcaga ggtgagatag actccagtct cctatagcac tcccaggctt attaggaaga |
| 1861 | ggaaattccc gcctaataaa ttttggtcac actggttgat ctctcaaaac cctgtctcct |
| 1921 | gataagatgt tgtcaatgac aatggtgcct gaaacttcat tagcaatttt aatttcacct |
| 1981 | gggtcctgtg gtcttgtgat cttgccctgc ctccgcttgc cttgtgatag tctattacct |
| 2041 | tgtaaagtac ttgatgtctc tgacccacac ctattcacac actccctccc cttttgaaaa |
| 2101 | tccctaataa aaacttactg gtttttgtgg cttgtggggc atcatagaac ctaccgacat |
| 2161 | gtgatgtcta ccccggacgc ccggctttaa aatttctctc ttttgtactc tgtcccttta |
| 2221 | tttctcaagc tggccgacgc ttaaggaaaa tagaaaagaa cctacatgaa tatcggggca |
| 2281 | attccccgat aaaatatatt atatagggag actcaaagca tatttctacc ttccctgtac |
| 2341 | taatttgaaa aagaatatat atggttgata aatatatata tatatttaga ataagtatta |
| 2401 | agccccaatg ttatgcatac tcaaaggaat tatacaacat aaagagaatc aaagtatatt |
| 2461 | tctactYtcc ttggactaat tcgtcctgtt ctggttgtaa gtatgggata tgaaatacat |
| 2521 | agatgaaatg tatgcttttg aatctatccc caatctcaat ttagttgttt caaattattt |
| 2581 | gagaatttct catttatctt tctctaactt gaattatgga gcctgtggct ttattttttt |
| 2641 | ttttttaagt tttttttttt ttggtcagtt gttgaaattt ttaaacagat atttggagga |
| 2701 | ggtgtatcag agccagctag ttttaagtct cagactggtc actcttaaaa taatcccacc |
| 2761 | cagtcatgcc atcatcaaat ttagttgcct ctctccttgg cttaaattta atacattttt |
| 2821 | tccccaatct attggactct ttagaaatgt tttaaacaaa aatgactaat ttatttttta |
| 2881 | aatgtaatta attttgttga ttgaataaga atgtttaaaa atgaaacatt attgtgaaaa |
| 2941 | ttaccacatc cttggtttaa aatagcctga gaaaggaaag cacagccata agaaataaat |
| 3001 | atacgaatag gttaatacag aggaaattgt gccagacaaa gctggttgct tttttgatag |
| 3061 | ctttacagcc ttagtaaatg aacagagtgg agtagacata atgcatcttg acttctagca |
| 3121 | aagtactgga gacaatgtcg tgtgaaattt tgattgcaaa attaatttga gttggcttga |
| 3181 | aaaacactgt taaaggaaat ataagctggt tgaaaagaca tacagagaag agtgttgatt |
| 3241 | aatgtcagtg aactaatgag ttgagtgata tctccagtta aatgtcacga ttgtgagtgt |
| 3301 | tggattgaat tatttttgtt atctgttgaa aaaataaata acatagggtt gattaaatta |
| 3361 | gagcagaaaa tttatcagga gacaccagtg attacagaaa atctatataa agacatgaat |
| 3421 | tcatttcaga gaaaaaccag tgaaacaata taattttact taaagagtcc atttttttaa |
| 3481 | tcacaaaatg gaattctatg aagtatcaat gcttaaagac cttcaaacta cagaatttct |
| 3541 | caaaactaaY tggaaaacaa aattaactat ctcagtaagg ctagaacatc attttaatcc |
| 3601 | agttagtagc tacattttat cccaaccaat aaagtgacag ttaattctta cctagaggat |
| 3661 | gtggttaaat gtataattat tatttaccag aaataaaaaa agtatctaga aaatcagtaa |
| 3721 | cctctagaaa ctcttgggca caggagaggg agaaaactat ggaaggggaa gtaattatgt |
| 3781 | tcttaactat gttacattct taagttacat tcttaactat ggggatatag taagactcta |
| 3841 | aataactaaa gttttttttt attatacttt aagttttagg gtacatgtgc acaacatgca |
| 3901 | ggtttgttac atatgtatac atgtgctatg ttggtgtact gcacccatta actcatcatt |
| 3961 | tacattagat atatgtccta atgctatctc tccccactcc cacctctccc cacaacaggc |
| 4021 | cccggtgtgt gatgttcccc ttcctgtgtc catgttattt ttgaaatatc cagattaaat |
| 4081 | gtagttaagt aaaacaataa actttttcaa tgtaggtttt ttaatgctaa tgaaaagcta |
| 4141 | gtcagcatgg ggtaaattag attaaattag attaggtaaa ttggaataag aatcaaatga |
| 4201 | tcacaatcat aaaatgggat tagtcttact ataagtcaca cctgcaaaag gaagaagata |
| 4261 | acctggaaaa atttaaagag tcttccactt tacgaagtcg tagtgagttg gcaagtttta |
| 4321 | ataatttaaa agaaaacgtt gatttcatct aaaaagagag agagaaagtt ttagttattg |
| 4381 | attcaacatg tatttattga ataacttaaa tgtgcactat ttttggtttt gtgagcagtc |
| 4441 | ccaaacgatt aaaacagagt gacttgttcc ctgactatct taaattatcg caaactgatt |
| 4501 | ggcttaaaac aacggaaatt tattctctca cagttttgga ggccaaaagt tcacaagcaa |
| 4561 | ggtatcagca gggctgcact ccccctaaag gctttggggg agaatgattt tgagcgtttc |
| 4621 | agcttctgtt agctgtcagc attctttgat ttgtggcaac atcactccaa tctctgcctc |
| 4681 | catggccatg ttgcctcctc ttctgtctga caaatctccc tctaccatag tcttaaacaa |
| 4741 | acagttgtca ttgggtttag tgcatattca tgtaatctag aacactctcc cctctcaata |
| 4801 | cttaacataa ttatatctgc aaagatcctt tttccaaata aagcaacgtt tgtttccaaa |
| 4861 | ttttaggatt taatatctct gggtgaccat gatttaacct actacaaaag cattttctaa |
| 4921 | caagcagctt aaaataggtt tcttttttct ttgagacagg gtcttgctct gttgtcaagg |
| 4981 | ctggagtgca gtgSacagtc atggctcact gcagcatcac actcccagtt tcaagcgatc |
| 5041 | tttccacctc agcctcccaa gtagtttaga ggacaggtgc atgccaccac acacagctaa |
| 5101 | tatttcattt ttttgtagag acagggtctc tctgtgttga ccaggctggt ctaaaactcc |
| 5161 | tgagatMtag caatcctcct gccttggcct ccYaaactgc tgggattaca ggtataagcc |
| 5221 | actgtgccag gctcaaaatt aggtttctat tgctgcatta atatatacac agRgccatct |
| 5281 | ctttctcttc taattatttt acaaatataa agagcatctg taacacaatg aagctgtttt |
| 5341 | ccactttagg aaaagacagt tttgaaaatg tgattaatta tttgttgcag gttaggcttc |
| 5401 | atacagtcat attcccagtg cacattgtcc tcaagatctc cccagtttgg cactgaagcc |
| 5461 | ttattgactc tgtctctgaa agaggcctta aatgatcccc cttccttcta atcctcactg |
| 5521 | aaattccctt aggttaagcc caatttatcc aagggctatg gtcttctact tgatgttctg |
| 5581 | tttttaatcc ttgatgctat ccttctcctt ccatacaaac agtagataca catattttca |
| 5641 | aaacacagac tttcttgtgt tctctactta aaaatctgtc tagctttcag atacataaag |
| 5701 | gaaaagcaac ctggttttaa gagcatgcaa tgYttcttct taaatttccg atccctgtcc |
| 5761 | ttgtctgcag atttatcact taacattcct ctccttgctt cataatacta cacacacaga |
| 5821 | catacacaca cacaaacaca cacaaacaca cacacacaaa cacacacaaa cacgcacaca |
| 5881 | caggcacaca cacacacgca cacacaaaca cgcacacaga gacacacaca cacacgcaca |
| 5941 | cacacacaca cgcacacaca gttccttgat attgcagtca cacagaattg ttcactgttt |
| 6001 | cttgaaaata atcataaact gcatttgctc atgttatttt ttgagatcag agtgttcttc |
| 6061 | cctctttttt ctgctttaag gttttctctt tttttgcaca gctattaaaa cttggccaac |
| 6121 | tattatattt tctctatagt tttccctgat ccttacacat cttcatagct ccttacatgt |
| 6181 | aactctatta ctgtgtgcat catatgcgat cgtgtttatt ttccctgtgt gatttgtttt |
| 6241 | ttagactatg accaccttga agacagaact gggtcttatc tatctttaca tctcctagtt |
| 6301 | gtttgcatgg ttcttgacac atagcaaatg ctcataaact tctgatgaac taaattactg |
| 6361 | taaattaaag tcattgatac atttaaatgg cacgatttac ctatatctgt tttataatca |
| 6421 | tagttaacaa attaagcaaa atacttctag gaccaggaac tcaatatgga gacactccat |
| 6481 | tgtaagagag aaagatgcat tgaatcgttt attcagaaag aacttattat gtcactgtag |
| 6541 | gcactgcaca aaggctaaaa atcccaaaat agaattgtta aaaaattagg gtttaagatt |
| 6601 | aaaggcaaag gaaatcatat ttgttgagac tattagggtt gcttgaagta aactaatgcc |
| 6661 | aagtttaagt aaaaagattc ctgctatatg atataattgt taagcctaag agaccagcag |
| 6721 | gcaccaggta cagttacatt cagatgctca agcaatgcca ttagggaact gcccatgttt |
| 6781 | agccattact gtgttctgtt attccttttt tttttgtttt gttttccctc tcaggcagat |
| 6841 | tctactcatg tggtaacaag actattattc tagatactgt actcatattt aacagtctca |
| 6901 | gggaaagagt gaagcctttt actcaatcgt tccagaaaat atctaatgga tgaagggaag |
| 6961 | gctctaactg gccttcgttg gaccacttac atttctttga accaattact ctggtcagaa |
| 7021 | gatgaacatg tctgatcttt caggattaga ttcacatgca aaataatgtt acaattcaaa |
| 7081 | ctaggaaaaa taaattatat atttttaaga gactggatga aaatactcca tacatcacat |
| 7141 | gtgcttttct ttttctgtat attggtatat acataatcac tatttctttt tttttttttt |
| 7201 | tttttttttg gagatggagt cttgctgtgt tgcccaggct ggagcgcagt ggcatgatct |
| 7261 | tggctcactg caacttccgc ctccaaggtt caagtgattc ttctgcctca gcctcctgag |
| 7321 | tagctgggat tacaggcacc caccaccaca cccggctaat ttttgtattc ttagtagaga |
| 7381 | cggggtttta ccatgttggc caggctggtc tcaaactctt gacctcatga tctgccagcc |
| 7441 | tcggcctccc aaagtgctgg gattacaggc gtgagccact gcccctgacc caccactatt |
| 7501 | tcttttgtaa ttaaaaagta catatacact ttatattttg atgtagatgt aagaatagcc |
| 7561 | tctgcttcaa agacctcatt ataatagtat taaagacaca caagtaaaca aaggtaacaa |
| 7621 | taaaatagta atacatatca aatattaagt atctcaggaa taattattcc ttctaaggaa |
| 7681 | tggcaaagaa atttttcaca ccacaaagtt aattttgaat tgggtcttca aggaacataa |
| 7741 | caatttgaaa agacagagag gactttccag gaaaatgtaa ctacatatgc acaaatgaca |
| 7801 | gtagggatta tagtaaKWac acttttatta ttattctacc tcttcactct attcaatttc |
| 7861 | cctgactact acaatgtccc catgttggtt tctcctttct tgtattccaa gatattgtgt |
| 7921 | cctgcttttg cttattattc tctacccctt aagcttctca ttgcttcctc cttcttttct |
| 7981 | cttttttctg ggcctccctc tttgttttta cgatccctgg gaggactcat ttaattgact |
| 8041 | cctaaatggt ttctgtgtct ctagtgtctt gtagacatta gaggcacaga aaccattttt |
| 8101 | cactgtctat tttccactcg gcatccaaac taattttcca taaacattgc ttccatcatg |
| 8161 | gatatgtgct ttaatagcat tggaatcaga cagatcttaa gcacaataca atagtatata |
| 8221 | taccagatac cagttatatg actgtgagaa gatagtaact tctgagagtc tcactactaa |
| 8281 | tgcccaactt gtaagataat tatgaaaata tgtacaacat atgtatcact ctcagcagtg |
| 8341 | ctagccacta gatagatgtg caatatataa gtagataata cttttgtggg tatctatatt |
| 8401 | actcctttgc ccataaccaa cagtgaacca tcatgttcct ttttaagctt ggtggtggat |
| 8461 | acaggtgttc actttaaaag tctcagtctc agatttcaag tcttcaaccc atctcctaag |
| 8521 | ctgtcagagg cctttacaat ctttacctac ctcctttatt cagtcttatt tcctatcatt |
| 8581 | tccccacagg aactatttgc tggcccttta aKttctccct tacctcagaa cttgctaata |
| 8641 | tttatcactc ttaaatatat gtttttgctt gtgctgttcc tcaaactaga atttttctgc |
| 8701 | catcttttgt tatcctctct cttcttttag tttaaatacc ttctgttctc taagtttttt |
| 8761 | ttttccagga caacctagaa aaaaatatct ctttagtttc tacagtggca gaaggtaatg |
| 8821 | tgatttacca atttaattac ttcatattac aatatagtga ccatttatat cagtgtatag |
| 8881 | tacaaatatg tatactgtgg tcttactact gattatctta ttcttattta ttgcctctcc |
| 8941 | aacaagatgt tatcacccat agacaagaat catattttat atatctttat aacccaaagt |
| 9001 | gcctttttaa aattaatgta tcatgtttgt tttcctggaa caggaactga aatagagagt |
| 9061 | atgaattcat ttgtaactga atctttgctg taatagttat ccccagcatc cttgctaata |
| 9121 | cagagaatga agccttaata cctaaagcag ccactgtRtg tgattaatta acttctctcc |
| 9181 | tatgcatctt gacaggtagt ggctatgtgc cattcttggg agaactggga aaatggtcac |
| 9241 | tgaaatcatt tttgttgttt ctagttccaa tgagaaatgc cagcYgggaa aggctgctac |
| 9301 | agtatgtagc aaggtgcttt tgctgaaata ataaaagtat ttgcaaggaa atacttgaaa |
| 9361 | taagtgctaa atttccaaag attgatctac actacagaag gcttattagg gtatattgat |
| 9421 | actagtatgt gttgtcttaa gtggtacatt gtagtttcca tgccaattct tggtttaaaa |
| 9481 | cgcagcatgc atctgttata tgccactttc cacttaaatg cctaagacag gtagattgca |
| 9541 | ctgtcaagtt aggtcattat aactccaaca gatgttgtca tgtagatgaa gaaaattcat |
| 9601 | tttaaatgat atcatttata aaatgtgttt ttctttgtac ctaaaaaagg caataatatg |
| 9661 | tcatattagc tcttcagatt caggttgcaa tcagtatggc atgttttgat taaacacttc |
| 9721 | acaatttcaa aatcagtgca ggataatgat ggagcacatg tgaggaaaca gcaatagcag |
| 9781 | gggtgtttac agtatcccag ttgtttcaag cttaataaca cttcatgtta tcaatcagtt |
| 9841 | acaaaggaat aattggtgga taggctgagg aaatggtaca gtaagaaatt gtacttattc |
| 9901 | taaactatag ggatggaagt gcagccaggt caatacttat ttggttagta tattgtaact |
| 9961 | aatcatttga gttttgccag ctggtatctt atttttgctt atttgtttaa ccgcttttaa |
| 10021 | ataagtatat aattttaaca atttaagtca gttgaaatcc cccccataga gctctcctcc |
| 10061 | aaatgcatcc ctagctacat tcttctttct ggagattaac cagaaattgg tggtttcatc |
| 10141 | ctgtttatat gaatacattc ctagaagata aaatcatatt gtagatattt gtgacaaaat |
| 10201 | tttcctcaca actatatgaa agagaacgaa ggaaggtgtg tggttaggag ttagctgtca |
| 10261 | ttaaaagtgt gtaaattttg cacgttccct acatttattt tccagtaaat tttactagtt |
| 10321 | tctttctaat cagttgggac taaagtcaat cgacctgtct ctgcttcttt caatttcatt |
| 10381 | tcccaaaaga attttagtgc ttctctatta ttcttaactc tgccttgccc caatacatta |
| 10441 | atcaatgctt tgggtaacaa cattagattt gtacagggaa ggaccaggga atggcattcc |
| 10501 | aaagcaacac atggaaaggg caagagctga atttcataaa ccgtaaccta atccatcact |
| 10561 | gcagctttcc gttgcctcta cccagactgc agccaaacat actctaagct gccactttag |
| 10621 | agtatcttta ccctccatgg gagtttttct ttttttccct tttttttttt ttttttgagR |
| 10681 | cagaatctca ctctatcgcc caggctgaag tgcagtacag gatcttgtct cgctgcaagc |
| 10741 | tctgcctccc gggttcaagt gattctcctg cctcagcctc ccgagtagct gggattacag |
| 10801 | gcactcacca ccacgcccgg ctaatttttg tatttttagt agagatgggg tttcaccatg |
| 10861 | ttggccaggc tggtcttgaa ctcctgacct cataatctgc ccacttcatt ttcccaaagt |
| 10921 | gctgggatta caggtgtgag ccaccgcgcc tggccaggag tttttcactg tgatgttgtg |
| 10981 | taggtctctg gaatgtatgt ttccctctgc agtttcccca gtgttgattt taagggagga |
| 11041 | agccagagca cttgctattt caccatcaca cttgtttgaa gtgtgagtta ccatgcaatg |
| 11101 | tttaaaacat gagttacacc tttttggagg atgctgtaga ataccgattt attttttgtg |
| 11161 | tgaaaacaaa ctctacctca ggttaatcaa ataattactg aagaaaaggt ttttctttat |
| 11221 | tcaaatattt cagcacatga atgaagaaga aatggtaaaa gtaaaacatg aacatttggc |
| 11281 | catcactaat tatattagtt tgtagaggct actgtaacaa aattccacag accaagtggc |
| 11341 | tgaaacgaca aaaatttgac tgggagttca aaagcaaggt gccagtggat tgggttttcc |
| 11401 | ctgaggcctc tctcttcagc ttgcagatgg ccaacttttc cctgtgtcct aacatggtct |
| 11461 | tctttgcacc cgatggctct ctttatggtc taatctcctc ttcctgtaac tacaacagtc |
| 11521 | tgattagttt tgggcccacc catatgatct cacttaacca taattatctt ctttaaagac |
| 11581 | cctatcttca aatgcattgt gaggtactgg gggttagggc gtcaacacct aaatttgggg |
| 11641 | aaacacaatt cagctcatta aatgaatgga agtcatacct gaaatcaatg acggatcgta |
| 11701 | tgactacaaa aaaaggagac aaacattata cacctgctgt ttaaagtata caacacaaaa |
| 11761 | ttattgacaa atattaagct gaattaggtc aatcttctag atttaccaat ttgcaaataa |
| 11821 | tgcaatccac agagcaagat gtggatatgg gggattcaga cagcaYaatc tagctcggga |
| 11881 | aactacagaa aaaaaaaatg acccattttg tgacatataa ttgaaggaac aaatgaagat |
| 11941 | agatgaatgg agaacccRta aataaagcaa ttcaagatac acatgaactc ataaaacata |
| 12001 | ggtcctaatt tgagtaaaga aagaaaattt taatactgac tggWtatttg atgacactga |
| 12061 | gaaactattc taactgtgca gctatgaaaa ggaatgagat catgtccttt tcagggacgt |
| 12121 | ggatgaagct ggaagccatt atcctcagca aactaacaca gaaacagaaa accaaatgcc |
| 12181 | acatgttctc acttataagt gggagctgaa caacgagaac acatggacac agaaagggga |
| 12241 | acaacacaca ctggggcctt ccccggggbg gagtgcagag agggagagca ttaggaaaaa |
| 12301 | taactaatgc atactgggct taatacctgg gtgatgggtt gataagtgca gcaaaacacc |
| 12361 | tttgcacaca tttacctttg taacaaacct gcacatcctg cacatgtacc tgcacatgaa |
| 12421 | ctaaaaataa aaataaatta ttctaaatct tgggaagtat gataatggtg tcatgattat |
| 12481 | gttttaaagg acttcttaac ttttagagat atataccaga atagaaataa tattgtataa |
| 12541 | tgtctgccat ttgtttcaaa tcaaatctga ccaaagtagg ggcaagaaag agtaggcgca |
| 12601 | taaataaata actcattgat aatgagttga taattgtttc agtaggatga tgaatatttg |
| 12661 | agtttataat tttattctat ttatatattt gaaaattttt atactgaaaa gttgaaaaag |
| 12721 | ctttatattg gtattttaaa ctttgtaaat gtKcactaaa agaagatatg tctttttagt |
| 12781 | tgttttgcct gagttatgat gagctttgat gagtcaatca ggctgcctct tgagttttcc |
| 12841 | ttgtaaggtt aattgggtgc actggagagc tagctgaggt taggagagca ggctctggtt |
| 12901 | gaaggacagg gtttcggctt ggcagactaa ctaccttctc tgattattaa actttgatgc |
| 12961 | cacccacatt agcacagtat tctagtcacc tgtgataacc tgtccagaag ttgctctgat |
| 13021 | cgtgttcagc tcaataagtt tagacttcac acaatagtgc ttgtcatttt ttaccctctt |
| 13081 | gtaccaaata tatttcaaaa tatgtccaat ccagctgtta atttgactga ataaaatatt |
| 13141 | ataataacac ttctgaggat tgagatgatg tttgaagttt ttcaagagtc ttttccctct |
| 13201 | ggattagaga acatctctgc cagaatcaaa atgagtctca aaattgacag tttttacttt |
| 13261 | acttgctaca gtaacctggc attttaaaaa gcattctgga aaaaaaaaat gaatcttctg |
| 13321 | attgtgtaat aaaagaataa gaatagaaac taagataaaa tgattctaca tgtatatctt |
| 13381 | gacttatgtc tatattttat aatttcaact tttacgtcaa aaaattgtaa aatgtcacta |
| 13441 | attttataag gtttaattta acagatcaaa gctcatagga ctttgttgaa aacaatgaat |
| 13501 | gcaaatcctc agtacagttc caggtgtgtc atgagccctc caaaaatgat agttacaatg |
| 13561 | actgtttcaa aaaatttata ccttcaacaa aactttaatc ctatttttat atgcctgtct |
| 13621 | gctctgaaaa taatttaata tgtttaaatt cctagaataa atcttacttt aaccaaatta |
| 13681 | ataattctta aacttaaatt tagatttcaa tgaatcccca aaatggtttc cacataaagt |
| 13741 | aatgcagcta atatcagcac tgctaacaga ttacacagaa aggtctatta taatgattga |
| 13801 | gtaaatattc ctgggaaatc aaccttgtta aatctatatc ctataaggaa aggtgcatat |
| 13861 | ctgttttttc tagtaaagat tccgtaattg ttattacaag ttaatcagct tctcccaggt |
| 13921 | tcatttactc tggattttat taatttatta caggaaatta ttgggtaagt aaaccacaat |
| 13981 | tataataaaa tctcatacat tttattatat ggttcacaaa ctttataaac acctgtgatg |
| 14041 | ggattctgct attgatattt aagtggatat ttattaagga actagagata cttctcagta |
| 14101 | agattagctt agagaacggt cctgtttttg atctcttaag attccaaatt tttattccaa |
| 14161 | attttctgat attgcccaag tgtcttttct gaatctatgt ctttattatt tggcttaaaa |
| 14221 | cctttgtgca tctggtgctc tattctgctg tgactttgga gaaaggttac agtttgtttt |
| 14281 | tgctgggagg aataacttct tgaatgatgt cccaactgaa aattattttt ttggttggat |
| 14341 | agaacatccc tgtacaaaac ttcctctatt atcccttgga taattcctgc tgtatataga |
| 14401 | ggtccaagat tgattaatga ttttatcttc agtaacctgt gatctaaatt accattggca |
| 14461 | agacaagagt gatacctgtt tttttattct ctacattaat aaaacatgcc ttaagaaatc |
| 14521 | tatatacttc atatagagga tatgatttgc cactaagcaa actctatgaa aaagaattgt |
| 14581 | gttttcatag taaggcttta ttataaacca aatatgacca actttcagct aaactgtatt |
| 14641 | atctaatcaa gtttattccg tcactttact tgctattatc aataagacaa gtaatatatt |
| 14701 | taaatataga ataagtaaca agcaacatgt taactgaatt aatatattgg taaaatatag |
| 14761 | attttaaaag ttttttgaaa atatttgagg agggttgcac ttttgctccc tctgatttta |
| 14821 | ctacgtatgt ctctgactgt atgtaatcac acctagctta tgctgtccct tccattctct |
| 14881 | acacttgact ttagaaagtc aaacgtagac tggagcaaaa aggggagaga tcttttccta |
| 14941 | ccatttagca gattaaaagg tagattggaa atgcatctca gtatctcagc cataaatgtc |
| 15001 | taagggataa taatcccgct acatatttat taaactaatt acaacatatt gcgcaaaata |
| 15061 | aaatactttg aatcagaatg acttatgaat ggtcgattgc atacaaaagt gctggagata |
| 15121 | tggtgataaa aagatgattc agccatcaca aagctcaaaa ttctactggg agaaacaggt |
| 15181 | aaagtgactg aagcccagga taatatttca aataataagt acagtgagat acttatttga |
| 15241 | gagatgcaaa ctcaacaaca gtttggtaag tactttctct attctaattt gaatacattc |
| 15301 | tagtcaattg agatacacaa agcatcaatt ctccccattg gatttggatc ccataaaact |
| 15361 | acccatttcc ccaaatagat agtgttttaa cttaacagac ctcataaaaa gcttgacact |
| 15421 | aaaatgagaa tttataaatt gtgatctgta gtaagttaaa tggtgcccaa ccctgcgaaa |
| 15481 | aacaaaaagt atgtccatga actaatccct ggaacatgta aatgtgatca aatagggaaa |
| 15541 | agatatcttt gcagatataa ttgtaaagaa tttcaagatg agatYgttat ggaatatctg |
| 15601 | agtgggctcc aaatctaatg gcaagtatct ttattagaga aagacatacg gggatttaaa |
| 15661 | acagaaaagg aagaggagac agccatgtga aaatggagac agagattgaa gttatgcagc |
| 15721 | cacaggtcaa agaatgcttg gaaccaagag aagctggaaa agacaatgaa agattttccc |
| 15781 | ctagagcctt tggatagagc cctgcaaata ctttgatttc atatctgtcc tctggaactt |
| 15841 | ggagagaata attttttgat gtttaagcag atgactttgg ggcagtttgt tatggcagcc |
| 15901 | attgaaagca agtcgtgatc tagtttgcct tattttccaa agcatagata attttcaatt |
| 15961 | tcaattagga aaattgaaat tccacaaaat gcaagtgcat acagaaatgt ctatctgagt |
| 16021 | tgcagttggg aaacattttg catttaaaaa ggaaaagttt taagatataa aagaaagtct |
| 16081 | tgagaaaaca agagaatatc cctcatatat gtacaacttc ttactaatag aagccttaga |
| 16141 | tgtctgatca atttcagagg caaaggctac cattgactgt tcattgacca ttgacaagcc |
| 16201 | atgtgtttca atgatgaaat accacataac aaaatactct aaagcatatt gtcttaaaac |
| 16261 | aactgtttta ttactaaggt tcctgcggat ctgatattca ggcaggattc agtggggata |
| 16321 | gcttacctct gttctacatg gcgctcactg ggatagttca attagggtaa gaggattgaa |
| 16381 | gatggtatca tttgcatgtc tggggctttg gtcctggctg ttggctgagg tgctttagtt |
| 16441 | acccttttct ctccacatag cctctaatta ctcaatattc taccccagcc tttaaaaaaa |
| 16501 | attttattgt ggtaaaatac acatataaaa tataccatct taaacatttt aagtgtacag |
| 16561 | ttcaatgata ataagtacat ttatattcct ttgcatccat catcacccat ctattttcaa |
| 16621 | aactcctttc atcatgcaaa actgaaactc tatacttagt aaacaatgat tccccatttc |
| 16681 | cctctctgtc cagcccgtgg caactgctat tctttctgtt tctatgattt tgaccatttc |
| 16741 | aagaaactta tgttaagtgg aatcataagg tatttgttat tttgtgactg attcatttca |
| 16801 | cttagcataa tgtcctcaag gttcatccat gttgtagtat ttctatcctt tgtaaggctg |
| 16861 | actgataagg agagacttac ttctgtcatt taaaaacttg ttttctatat ggcttatagc |
| 16921 | atttttgtcc cttgcttcct gcgtgactgt cttcttttgt gtttagttga ttattgtaat |
| 16981 | gaaatgttta tgtatctatg tcattgactt ttgtatattt tctatagcta ttacatttaa |
| 17041 | catcctacag ttataacact cttatttaga tttatgccag tttaactcca ataatacaca |
| 17101 | aaactctctc ttttacattt ccatacccac ttcttttggt tgctgatgtc ctaaaactac |
| 17161 | acctttacac attgcaggtc caaaaacaaa aactaataat ttttaaatat atcagtttct |
| 17221 | tgaattaaac cattgttaca ataatagtag cttttataat tgtccatata cttaccttta |
| 17341 | atcttcagaa ctcacattaa cgtttcttcc agacaagtct agtgttaaca aactcctctc |
| 17401 | aacttttgta tacctagaaa tgccttaatg tctcccttag tttgaaggaa atttttgcca |
| 17461 | gatataggat tcttgcttga caggtttatt ttctcttagc actttgtata tattggccca |
| 17521 | tttctttatg gtttccaaaa tttctgataa gatatatgct tataatttta tcaaaaatcc |
| 17581 | cttgtatgtg attaatcact tctcttttga tgtttttgat attctctttt ttgtcttcat |
| 17641 | catttcaaag gttgataatg tggatctctg agctcattgt atttagagtt tactgagctt |
| 17701 | cttaaatgtt tatattcata ttctttgtta tatttgggag ttttggccat tatggcttca |
| 17761 | aatatcctct cttcttttct ctgtcttctt atggactcca taatgcatct gtttgtccac |
| 17821 | ttgatggtgt accaatggtt ccttattctc tgttcacttt tctttaatct tttttctttc |
| 17881 | tgttccttgg attgataatt cccatggtct tctctttaca tttgctgagt ctttcttctg |
| 17941 | gctgcttaaa tctacctttg aaaatctctg gcaaattttt ttcatttcac ttgtactttt |
| 18001 | tagctacaaa atttctttct acttttaagt tttctatctc tttatggacg tttataattt |
| 18061 | attcatgtat tatttcttga tttcctctac ttttctttta gttctttgag catctttatt |
| 18121 | acagttgttt tattacagtt gtccagtagg tcccccatca tgtctttttc agtgacagtt |
| 18181 | tctgttggct tattttattt tatttttaaa tgagccattc tttcctgttt ctttggatgc |
| 18241 | cttataattt ttttttgttg aaaattagac atgtgactgt agtaatgtag taactccggc |
| 18301 | aatcagattg tacttatttc tcctaggttt tcctaggaga aatttcctag gttttcccct |
| 18361 | tttgttctaa atgttgtaga ctagactgtc tctgtgcaaa ggatcaatct gagatgtacg |
| 18421 | taaatttaag gtcttctcaa gtctcttttg agcctgcacc tttccctggg catacacagc |
| 18481 | aactttctaa tttcccgtgt gtatgtggtt gcttttaaat gtcttagtat ttaatgtcta |
| 18541 | cctccaaaaa gaggaaaaga gaaaaatgaa ggtggcaagc aggcactggc catttaaatc |
| 18601 | ccctggaaat tacttcagcc agaggggtag gggtgtgcaa caatggaggg gaggtgcaac |
| 18661 | aacagtgact acctagccct tcgtctacaa gtccgtgatt agaagcagca gtaagcaatc |
| 18721 | agagcacaga tctccaatat ttggattata acattccttt tgcccatgct ggcttttgtg |
| 18781 | aactgcatgt aagttgctcc tgaaacatat tcatgNctgc ctgccattgg actaagggtc |
| 18841 | aggataggta gctgctactg ttttaagagc ttaaattgat caaaagtaac cataattttc |
| 18901 | cttataagct ttccactgga agtttcaagc cttcaataga ctccagaact ccagaatatt |
| 18961 | tatatcagat agattctgcc agtgcagttg ttgcctacat ggggagacag atttctggtg |
| 19021 | ctttctattt tgccagcttc ccaaaatctt cctttctaat cttgtttctc tccttgaaag |
| 19081 | ctggggagaa ccagctgtaa ttctaatttt tgttgttagt ggactatggc tgtgagaaat |
| 19141 | gtcctctgga tggggatgcc actaccccct acacagctca catctcactc acaactttgg |
| 19201 | ttttcccctg ctctggacca gccttttttt atttctttat ttctttttca cttgattggc |
| 19261 | ttccaaaaat gagaagagaa aacctgtgag atctcttaag attcatgcca ccaaatcata |
| 19321 | tgtcatcact tctgccacat tttactggag aaaactcaca gagcaaactc agattcaaga |
| 19381 | aaaggggctg tcttagacct tttgagcttc tataataaaa taccttagtt tgggtaattc |
| 19441 | ataaatagta gtaatttatt tctcagagtt ctagaggctg agaagtcaag gcatcagcag |
| 19501 | attcaatacc tggtgaaggc ttgctctctg cttcgtagac ggcacatctt gcagaagggg |
| 19561 | tgaacagtat cctcacatgg ccaaagggga aggcaacttc cttcagttca ttttataagg |
| 19621 | gagctaatcc tattcatgag ggaagaaccc tcgtgaatta atcaccttct taaatcttta |
| 19681 | atacaattga gttccaacat atgaattttg gagcgcaaca atattaggga agtggactcc |
| 19741 | atctcttgaa agaagaatgg tatgtgtgca cagggatagg aggaattatc tgtggccatc |
| 19801 | ttgcaggcag actaccacac agtgctctgc tttttcttat cattttcttt gatgacatct |
| 19861 | gatcctttct cacaaatatc tcctttgtaa aacactcagc acttattatt tatatcagtc |
| 19921 | atttgggaat tagtcatatg ctgcctagtg gaatttgttg tcattaaaaa ttttttcctg |
| 19981 | ttttgtcatt tcaaaaacaa tcacaaactc ttggaggtta aagatgatgt tctatatttc |
| 20041 | cttgtacccc gtaaaaccag catagtaggt atttaataaa taactgtctt cttaattata |
| 20101 | aattttaagt ggtgggagaa tgatgtattt acactaatgc acggtggttg tattattatc |
| 20161 | cactagattg tttcttgctg catatcatgc actgcattat ttcttgctgc atatcaatca |
| 20221 | cttagtactg acctcctttt taaaatacgc ctgactttct catgcttatc ttacatatat |
| 20281 | ggttatagct aagagttgtg aatggacaca gatatgggaa gcatatttag gaggattaac |
| 20341 | aaaagagtaa ttagaagaac acataaatcc acaaggaaaa aatgactctt tttgaagtat |
| 20401 | gctagcattt tcaacatgcc tgttgagtta atgatttatg ggagtttcaa ttgtttaaat |
| 20461 | gtttggaata ctctgcaata aaataaaaat tctcttcaaa gttgataaaa atactcagag |
| 20521 | gcatacttcc tcatctgatg attcatagtt accaatgaaa cccaagaact ccatgcacat |
| 20581 | tttcagcaag agcaaggaat ctgatagcaa gagagctgga aaggaagaat catacagtga |
| 20641 | cctagacaag tgtcatgtcg gtcttagcat cagataaact gattgcaatg gttgattgta |
| 20701 | ctatctctag cgagagtaat ttagaaaacc tgaataagaa accctctctt gttcatcata |
| 20761 | gggatgactc ttggtgtgaa cccaatcata ttaaacaatt gtagaatcac caagctagac |
| 20821 | attttatgag actaactgat ttattcctca tcttgaaata aaacctgcat gccacaaaac |
| 20881 | tcctctatgc tgctgagact tagttctttc tttgagaaaa ataagagcca tttattcatg |
| 20941 | caataaattt tcattgtgtg tgaattgtat actaggcctg ttccatgtgc ttgccatacg |
| 21001 | tcagtgaata gaagaggcaa agatctttgc ctatggaact tgcagtctag taggggacat |
| 21061 | aagttatttc gtaagcttcc cctttataaa tatcattaat ccttactaat aataatactt |
| 21121 | acatcaatgt ttatatattt tacattggtt ttctgataat ataaaaaaaa attcacaaaa |
| 21181 | taaacccaac cactaataag ctgtgagttt tgagtgtaga attcttttca ttaacaaacc |
| 21241 | atttcatttt cagatcgaga aactaaggtt tttaaaaaac tgattttact gaaaataaaa |
| 21301 | tctaaccacc catcctaatg ttctttactt ctttatctgt ctttcaaaaa tatcttcctt |
| 21361 | tctctatgtt gagttagata tgtattcaca aaatctacca tactgtccat atatgttaga |
| 21421 | tataaatttt tgagctcatg tgaaagaaac caagtttgaa tagttgaagc aaaacagaag |
| 21481 | ggtttattat taaaaaaaaa atgtgttttc tcaaggaccc caaaacacaa agctgaactt |
| 21541 | gagaagtgag cgctcaaaac tattaaggct gtctgcatga cccttgctct aattcctctc |
| 21601 | tgaaatagtg tctcattgat tgaggtaaaa aatgacagca gtttctagag ctacactgcc |
| 21661 | tgtgttcaaa tcctagctat gtcatttgtt agctgagtag cctggaacag gtaaattaat |
| 21721 | ccctctaatt ctcaatttcc acatctgtta aaattagcat aattataatc atatcaggat |
| 21781 | aattctgaca ggattatagc aaaaaaagat taaataatat ctaatgactg gcacaatacc |
| 21841 | tgcctcataa atataagtct tcagtaaata taaaccacca ttattaaata ggagatgtta |
| 21901 | agaattgagc tgagctgggg agggctgagc tgccagatgt gggagtattc agtaagacaa |
| 21961 | caagccaaaa tgaaagtaac gattaagctt ttattccact tactgtgata gtataatcaa |
| 22021 | gagtccagaa agaaagtgcc agcttcacct gtgtttcatt ttccctacaa atggcaccac |
| 22081 | gcaagggtca gatgcattag tacagttttg gataactgtc ttactgctca gggaacccca |
| 22141 | aacaaaaagt tcctatggtt ttatggacct aagggcagga gaagaagcaa ggggaagatc |
| 22201 | taggaatggg aaagtaccaa gtactaagtc agaatggaga aaagtgtcct ccagggtacc |
| 22261 | cactcctccc aacaagcagg tcttggcaga ggctcctgga aaggttcatc tcagagtccc |
| 22321 | cagtaaagaa gcctctgaat ggaggctcct gggctaagaa tgcaaatatg tataaaacat |
| 22381 | ggctggccag gggtcctaag tccttgactg caactcccgt cagacactgc agcacattgt |
| 22441 | ctgtgcaagt cagtttgggg agggaagctt tcctctgtga aggcctccca ggcaaagcct |
| 22501 | ttgtattgcc tagggcaggg caaaatcata cagaggtttt tgacttggag ctggattcct |
| 22561 | tgagaggatt tctaggagaa atcctcccaa aagtttcaac agtttatatt tcatttgtat |
| 22621 | gttggtggtt ctctttcaag tgagatataa tatagatgaa aagtatctaa catagattag |
| 22681 | gtattactct tattattact atagctatgg ttgcttgtat ataactgttc agattatagc |
| 22741 | tccaataaaa caacttattt tattctctcc agagttaggt aatatcatta ttcctcatac |
| 22801 | tgaaggcttt tgaaaaatgt tccttggtct ttactataca gagtttaacc tttctgctct |
| 22861 | caggaagaaa gacagttgga aattcaccat ctaaatttaa aagggggaaa aatctcagct |
| 22921 | gatagctggt aacacagtgt tggctatgta ttgagatact tacttaaagc atcatataag |
| 22981 | aaatgatgta caggccaggc gcggtggctg atgcttgtaa tcccagcact ttgggaggcc |
| 23041 | aaggtgggtg gatcacctga ggtcaggagt ttgagaccag cctggccaac acagtggaac |
| 23101 | cccatctcta ctaaaaatac aaaaattagc caggtatggt ggtgtgtgcc tgtaatccca |
| 23161 | actactcggc aggctgaggc aagagaatcg cttgaacctg ggaggccgag gccgaagtga |
| 23221 | gccgagataa tgccactgca ctccagtctg ggcaacagag caagactctg tctcaaaaaa |
| 23281 | aaaaaaaaaa agaaagaaat gatgtacaat cactttaatg tgtattaagg ttcttgttta |
| 23341 | aaaatcatct aaatttgttt agtatccaat aaaaattgaa gtctcacttt gtgccaataa |
| 23401 | ttatgtattt aataaatggg aagaggaaac aaaacagtaa tttttcttga acacgtggaa |
| 23461 | atttatagat aattgctgca ataaatatta tcatacaaaa tatatattta aaaaacttaa |
| 23521 | ctacaatatt actgtaaaag agaggaacat gattctatat tacagtgata tgaggtagcc |
| 23581 | tggggtgggg gcaggagatc aaggagagtt taccccaaac tacaggtgtt tatattgaga |
| 23641 | tctaaaggat gaggaaaaat tactttacta attcattaaa gaattgcagg tgtcttaaga |
| 23701 | ttgttccagt caaggaaaac tgtatattca aaaccactga ttctcacatt tggctctact |
| 23761 | ttggaatcat cttcataact ttacacaata ctgatgccta gatttcatgc ccagattctg |
| 23821 | atttcattgg catggggtgt ggctttggtg tcgggatctt tttttataac tcccctagcg |
| 23881 | gttctgataa gctttagtca gtgctttaga aacaatgttt aaattttcaa ggtaggaggg |
| 23941 | ttatgcctta ctcaagaaac cgtaggaaga ctcatatgat ttgaattcaa agaatgagag |
| 24001 | ggaaggcatt atgacatgaa tYgatgaata taaataataa caaatcatgc aaattctggt |
| 24061 | aggccacatc caaagtttgg cctttatctt gagaatactg gaaaactatg gaaagacttt |
| 24121 | aagcagatgt tataatctca gaatttatgt ttgcaaagaa agactgtaaa gagaagctat |
| 24181 | gaaattagct tcggctagga tggtaggaag gaacgagtta aatagattca ggagacattt |
| 24241 | ttaaagaggt gaaattggca gagctgggca aaggattgga tctgagaggt ggtgagactg |
| 24301 | acagcttgat ttttggctag tgggactgag ctgatagtag ggatagatag tgagcttaat |
| 24361 | cttagagata tctgagctcc ctttgaggta tctaagcaga gacagtgaag tgatagtgaa |
| 24421 | atataaggag attgctgagc ttgagatttt tattgtttag gagtaaagat ctaaattgca |
| 24481 | atagagatgc tttacactaa ccttgtcagt ttcataaaaa ttactttact ggtacaaaac |
| 24541 | atggtaaaag gcactgtaat agacagaaaa atgattcaga tggtacccat gaccttaagt |
| 24601 | ggttaattat ttaatggagg agataggaaa gacagtgaga atttaatgtg attttaaaat |
| 24661 | gaacatctta aaatgtctca aggtagaatg gaatagaaga gaagaaaatc cagcaacttt |
| 24721 | tatcaggttg taataattaa atgctaaaat attattagaa aaaataataa tattgtgatc |
| 24781 | tgatagaaaa ggaaagctaa tagaatttga gggaggggaa tgaccccgaa gaatcatgaa |
| 24841 | aactctcagg gatacaacat catattagga cacggtgtct agttggaaac attttggttt |
| 24901 | ttcattcagc ttgaactgga gaaccaaaaa gaaagaagaa taaagaggac agcaggagtt |
| 24961 | ttaaatcatg gatttgttta ttcaaatctg ccaYgtaaca atacaacaat actatgataa |
| 25021 | gagctaggag tacaactgtg gacaaggtag tcatcattct ggcacatagt ttataggttc |
| 25081 | atatcatatt tattataaaa aatagtttac ttgagacaat atccctagtt agctttcaca |
| 25141 | aaggaaacca gtcaaaataa tgactttatt aaaagaggtt aagtagccat tctatgagtt |
| 25201 | tgcattccct gtgtgttgaa gcaagttttt ttttttcttt tttttttctt tttttgagac |
| 25261 | agtcttgctc tgttgcctag gctggagtgc agtggcaccc tcggctcacg gcaacctcca |
| 25321 | tctcccaggt tcaagcaatt ctcccgcctc agcccccaaa gtagctggga ttacaggcat |
| 25381 | gtgccaccac acctggctga tttttgtatt tttagtagag acggggtttc accatgttgg |
| 25441 | ccaggctggt cttgaactcc tgaccttagg tgatataccc gccttggcct cccaaagtgt |
| 25501 | tgggactaca ggcgtgagcc accgcaccca gcctgaagca agttttaggg cagtggtttt |
| 25561 | caaccttggc tatacattgg aattacctgg aaaggctttt aaactactga tgccttggtt |
| 25621 | atactcctgg ggattctgac ttaattgtcc tggggtttgg atggagcatc agaatttttt |
| 25681 | aaagctttcc attctaatag tcaacaaaat attctaatag tcaacaaaag ttgaaagcca |
| 25741 | cagttttaag ggcatgtttt atcactcaga ctactaagtc acccgaaaaa tgagactgag |
| 25801 | caaggaacaa aagaattctg attatatagg aaaaataata acaaacattt ctttagtctc |
| 25861 | agtgtgtcat gaacacttct aaacacttaa catattttaa atcagttagt ggtcccaaaa |
| 25921 | actgtatgag gaagtcactt ttactattcg catttgagag atgagaaaac tgaaacccaa |
| 25981 | cagcacacgc aaagtaagtg ttagaacagg tacttgaact caatcagtct tcttctagag |
| 26041 | cttcttatct gaactggtat gctatattgc ctctaaatgt agtgtaatgg taattttata |
| 26101 | ccagaagaat tataaaagaa aggaaaacct ataaaaatag gatatcatta aatcagttaa |
| 26161 | atgctagttg ataatagagt gattattatg actgcataaa acatctatgg gttatacata |
| 26221 | tgtgtatata gacatatata tgtatatatt tattcaaata tgtctatata tagatatatg |
| 26281 | tatacatatt tgtatatata tgtgtgtgta tatatatgct ggttctcatg ttttcttcat |
| 26341 | ttgtaggaat cagttaacaa gttcttgtta catagtatga aggttctgtt ttcagtagtg |
| 26401 | aaacagggag ataaaagtgg ttgaaccatg tgattttttt tctgactttt caaagacatt |
| 26461 | ttctcctgaa ggaagctcaa gtgctaaggc tattccacat aatctcaaga acgactacta |
| 26521 | ctttatagta atgaccaaac tgtagtaata gaatcttagg agcatataat catcctctag |
| 26581 | ggaacacatt ttacaaattg aacctatctt gcaggtggaa ataccataaa gaaaatKatc |
| 26641 | attggagaga ttactggcac actgcattca ttcacatgtc aaagtggaaa acattccaat |
| 26701 | ttacttctat aaaacatgta tctgagtcaa cctgccatat gtatttttta cactttctat |
| 26761 | ccccttacag aatatctacc aaataaatgc tgcacatttc taagtggctg gcttttaagc |
| 26821 | tgctttgcct atggcttttt gttccctctt tttctcagct gagaaatcaa gtaaacaaaa |
| 26881 | cattgtggct ggtagcatag gtcctcagtg cagtggatat tttgtgtccg gcagctggag |
| 26941 | cattggtcgt acagatgggc ttcatttgtt ccatatgaat acaagttaat aaatggagag |
| 27001 | aattccattt ggtattcaat taagatctcc cctgactcct gttattaaag cctacaaact |
| 27061 | attgagcata aaattaaaat cgagcagttc agctcttttt gacattataa cactgcctga |
| 27121 | aaaataatgt aaaacccaag agtcacaagt cattccttac agcccatggc aaataagaag |
| 27181 | tggaaacatt gaggaagaaa gagtaaatat ttaaaacttg atcattagta cacatgttag |
| 27241 | gagcaggcta ctgtgctcat gcaaatacat caatccagtt tgttgaacac aagatattac |
| 27301 | attaatttta acagaatgtt acaagattat tattgccaaa gctccaatga aggaaataat |
| 27361 | taaagcacta ggcattttaa ggtcttaaaa tgacagaaaa tactatagga aatcaataac |
| 27421 | cagaaaatac tatagaaaat caatatactg taattgaaag gaaattgaat tgggtttact |
| 27481 | ttttttatcc cgtctaaatt ttcttcagat tgccagacca ctgaagatag tgaatcttgt |
| 27541 | tttgccatca gcagtgtgac tttctctaag tctcatgtgc tgcaagggcc atctttattg |
| 27601 | ttttttattt aggaagcact cagctgaata gacaStatga gattttgtgt gtgtgttctc |
| 27661 | tttactagtc ttggactcat tgttaaaaaa aacagaaaca aaatcaaaga cacagcagac |
| 27721 | aaaaacaacc acctccacat aaccagagaa gtcaaattaa tgagaattca gacatcgaaa |
| 27781 | ccagaggact tgttagataa gcctcacact tgaacctaag tgaaattctt ctagttttta |
| 27841 | tcaaacacta tgcctgtcat tcttgctatg caataagaaa cattaccagt tgcagataca |
| 27901 | aattcaattt gaatctatat gaatctaact cattttcatt gcttattatt ttaaaaagtt |
| 27961 | attataatca aacaaaaaag cttgaaatga acctcaggga ggtaagtgga tatgaacaga |
| 28021 | aattaaagaa tgttaaaatg agaaatccag actgggtgct gtggcttaaa cctataatcc |
| 28081 | cagcattttg ggaggctgag gtgggagaat cccttgagcc caggagtata aaacaaaaca |
| 28141 | cttaacaatt ctgagtctca atgtcgttga ctataaagta gaaatatctt cccttcctac |
| 28201 | cttaaataga tcacatttat gaaaaagatt cataagttat aaaacaacac ctcaatgaaa |
| 28261 | aatgctgtat ctggacttta aaaagaagtg aattaaatac taattttacc tcttagtata |
| 28321 | tatgtgatct taatgaagtc cttagttttc ttgtctgtta aataataatg gtaagggcac |
| 28381 | cttctcatat ggtcattgtg aagcttacat gtataaaaag ctcagtacca tggctgacac |
| 28441 | atagaaagta tttaacagtg ttcaaaaaca taatcaatat aggaaataag tattatttta |
| 28501 | tttccttaat aatttcttag ttatacccca ttatcatatg tgataagaga atgtacttta |
| 28561 | tttctagttt tccagataat ctgtatccta aacattcttt actcccaaat tcagtctttg |
| 28621 | agcatcctaa ctcttgacat ccacagctaa tatttaggaa acattaaaat ttgtttgtac |
| 28681 | atttgaagga agaactaagt aaatggaact taccaatcct aaaatccaga ctgctgttat |
| 28741 | taagaagaag aacaactgta caaatgctgt taYcaaagga tgtatgagtc aggcggaaaa |
| 28801 | aaaaggagaa cacagaaagg tgaaaagtct ttgtcaagca gaggagagaa attattgtct |
| 28861 | tgtaatatat atgagagagt ctagccaaat attaaacatt tttaaaatga aaagaaatat |
| 28921 | tatacatggt atgaataata tttaagtcat tgcctaggtt agaagattag atatatttac |
| 28981 | ctattctacc tacttcaaat gcttttttgc tgtcattgtt tggaacctcc aaatactaga |
| 29041 | atgctttaga attataaaac cattgaccat gttcagttat gtttaaaaga aaacatcatt |
| 29101 | ctgaggaaac tggaaattat atatttataa atgtttgtcc atagcattgt tgaaacaaat |
| 29161 | ggctcaggtt tcactgttaa tccagttgct gatatttttg actactcaat acaccaaaat |
| 29221 | ggtacagtca agttggtgct aatagatgtc atttatttac acaaactgac aataggttca |
| 29281 | ccaaagttaa gagacgaatt taagggggac agaaaagaaa ttcctgtgtg tgctttggca |
| 29341 | gcttagacat acttaccacg aacaagatta tatcttaaaa gctattcgtt aagcttaaaa |
| 29401 | ttaagttata tggttgtaat atgtggagaW agcacattgg aatagctctt cacttaatgt |
| 29461 | tatagatcac tttttccacc aatattattt ggtctaggaa agtaaaatcc tcttgaggaa |
| 29521 | gtaattagaa aaagcacaca tcagggaatg taattgaaga tagacttttg ttgacctcag |
| 29581 | aagagaaaag ctcaaaagaa atttcgtaaa gttttaaata tggcaagagg accacaatgt |
| 29641 | ctttgataat tagactttca acacatttgg atttgctaat tggatctaag ttctaattag |
| 29701 | gctttttatg taactaagaa tgacatatca ttgtatgaat ttaatgtgct tttacagttg |
| 29761 | aaactaacaa ttagcagcct tttccaaatt attttaataa ggaaaaagct ggctctgctg |
| 29821 | tttatactgt gacattccct tctcttaaga tatattatgt acattttcat tttctWaatt |
| 29881 | aaacaccttt tggggagctt aaagggaaac ttgggtttta ataggaagca cctaaattgt |
| 29941 | ttatttgtgg gggagacttg ccagccagta tgtagatgac tttggaagct acagacaaac |
| 30001 | cgcaataaaa agaaataaat cacaatcatc aggaggaaac agcacaaata accatttgcc |
| 30061 | cctatgacca ttcagttaat caaaacagtc tctgaatatg atgaaagatc tgttacattt |
| 30121 | ttaagtgtgc acatgcggaa tatgttccca aatgtgaagg ctcatggatc tgctttcagg |
| 30181 | atgaataata ggaaatttct tctcaagatg tgcattacaa cattctctct ctctgtagcc |
| 30241 | ttgttctgta aacagttatc tgctaattca agtcaaatca tctctttctt tctccatatt |
| 30301 | tttgattgag agtcatcaat ttttatccca ttcagatata tagacagtaa tttaaaaact |
| 30361 | ggtRgccctc agatttgtac attccaagag ataagaccta tataacagca taaaattaaa |
| 30421 | ctactatttg ggtgagtaaa gagcagtgag tcattgcaag actttaaggg aaaggctgct |
| 30481 | gccaaaatat tccaagcaaa ttgataacac tgccaaaaag gcaaattata ttgttttatg |
| 30541 | tgctatgaat actttgaaaa caaagacata cacatgattt ttttcaaata aaagcagttg |
| 30601 | cccttctaac catattaaaa atagtgtctt ttttcccagt aaaagccata taagatcaaa |
| 30661 | ttaaaaccat tgaaactatt agagaatcta acttacagta attgatatat taaaaaagac |
| 30721 | attatgttag agttatatgt gctattattt ttccaattag tacattactg ggactgagtg |
| 30781 | attggtgtat acataagtcc tcattaagta aaatgtcctc aatgagcctc ctaaagcaca |
| 30841 | ttgggtactg tcatgtgcca tataatgata tttaagagca agaaatggca tatctaataa |
| 30901 | cttcatttcc tatttaaaaa gaaaacctgt gcagtactct ttccatacca attattctaa |
| 30961 | gagctatcac atagttctcc ataaagacta tacagaggtg atgggactgt tcttcaggca |
| 31021 | gaaaaatgac caaagttgtc ttatctttca gcttggKtgc tggaatcctg tcacaggtct |
| 31081 | gaatgggtca ctgactgaca agaaattgga gaataacatg cgtggagtgg ttctacgtgt |
| 31141 | agtaactgtt ctggtaagta ttatctgagc ctcgtggttt tgacttttgg aggttacttt |
| 31201 | atcctatatt tctttcttta ttaactgatg acataatctt aaaagagatt ttaacaaatt |
| 31261 | ctaaatacgc tttctaagat tttttaatgg tcatatttca cagccttcaa tttacgtgga |
| 31321 | tcaggatcac agttttgcag acttttaact acagtgtatg tgttaggaat tttcttagaa |
| 31381 | attttaggct caatttccat cctacagttt tctacacatg acaaagtaaa ggtatgtctc |
| 31441 | aggccttctt tcattaaaac tgtagtgaac atagatagtc aacatatagt gaacattgaa |
| 31501 | actataaatg aacataaccc tgaccatgcc aaacaatggt agatttggta aaatgtacat |
| 31561 | tctctaatag tgaaatatta acaaactaag aatctttttc tcgtaagaac ctacttcaaa |
| 31621 | gggaagctgt tgtttaatag gcatctactg tcgactgtat tccaggctca gtgctgatta |
| 31681 | ctgagaacat tgacaaataa aatgacagat ataattctgt cttgatagaa cttatcatct |
| 31741 | agtgattctg tgaatgttga ataggattac agtagtttcc cYttatctgt gggggatgca |
| 31801 | ttccaagacc cccagtggat gcctgaaact gtggatagtt ttgttttctt ccaacacaat |
| 31861 | cacattgtgt agggcttata ggttatagag tgtggatatg ctggacaaag ggatgagtca |
| 31921 | catccagggc atgtcagtgg gatggtgtga gatttcatca tgctacttag aacaatgtta |
| 31981 | aattcaaaac ttataaattg tttatttctg gaagttttca tttaatattt tttggaccat |
| 32041 | ggctgattga ggagtaaccg aaactggtga aagagcaacc atggataaga agagtctact |
| 32101 | gtatacctgg gaaagatctg ttacaatttc atttacttat taaaaagtca ataaatggta |
| 32161 | actgttataa ttctatacat tatagttttc tttcctaata gatacataca ataagatgtt |
| 32221 | aatagaaata ttcttaaaca gtaaaatact caaatgggct ctgtctaaat agccataaac |
| 32281 | cacgaaggca tacattctcc tttgctattc caatgtgttt aatcatgtgc tagtttatag |
| 32341 | cctgtttcaa aacgtaatag tccatagtta gtactttatt tttcttcaga aaagtctact |
| 32401 | tgtttttaat tttgaaaaag attttcctat gggctaggta aattgcagaa ttagtaaata |
| 32461 | ggagaggaag actttttgat acgttgatgc taattggatg ttcatcaaat tttataacac |
| 32521 | cagttgctgc cctttgtaca ttgtttacta caaattaaat atatgcaatg taattttatt |
| 32581 | gtcaaaaaag tggatttgtg catattttct atgaaaggac attcaagtga gtgacctatt |
| 32641 | cttctttttt tgtggcccat aaattattcc tagttattat ttttaaaagg gcttaattgc |
| 32701 | ttttggtaaa taattgtaag tatttcacaa cttaaatgtg aattcgcttc tgtatgtacc |
| 32761 | taagggtatt gatatgcatt tacattgatg cactaatatt aaaaaagaca gatatttaaa |
| 32821 | gtacagcaaa ttattaatag tctttcctcc agtgattagc atgaaaactc cagtgatgat |
| 32881 | tgcagtgaaa gacggtatat gtgctttgtt ttcctttgag aaaagaaagc aaatctttaa |
| 32941 | gtccaattaa cctcagtcag ttttaagcaa atagttattt cgcaattact atatgataga |
| 33001 | ctcagtaatg gatataagag aaataatata cagaccttgt cctcaaagta ttcatagaat |
| 33061 | cttgttaggt tgatgcacat agagaaaaac agctcagatt gattaaatat ggttgaccct |
| 33121 | tggtgtctgt gaggcattgg ttccaggaaa cccccacacc aaaatctgca aattcbtaag |
| 33181 | tttctgatat aaaatgatat agtatttgca tataacctat gtacctcctc tcatatgctt |
| 33241 | taaagcattt tagattactc ttaataccta atacagtgta aatgctatgt aagtagtggc |
| 33301 | tctaccgaat tatatattta tttttgttat ttttattggg tttttttcct aatatttttg |
| 33361 | agcctcagtt tgttgaatct atggagttga gtctagatgM agaacctgtg gatatggagt |
| 33421 | gttaactgta cttactatgt gcaagctact ctactaagtc ttttgcatgt attgtcttat |
| 33481 | tttgtcctca caatattctc aagaaatagg taatattctg atctcaagag gaaactgaga |
| 33541 | catccagaag ttgaaataca tacatgtatg tgtgtgtgta tatatatata tatatatctt |
| 33601 | atcattaagg gaaggggtta aggtctagaa tcagacttcc tgaatttgaa tcctaactct |
| 33661 | gctacttact atctgtataa tgttcataaa tacatgaacc tagaagaaat tgaagaataa |
| 33721 | catgcaagga gcgattctac gtgtggtaac tgctctggta agtattacct gagccttgtg |
| 33781 | gttttacaga cagtaagtag cagagttagg attcaaattc aggaagacag attctagacg |
| 33841 | ttaacccctt ccctcttaat gatacattat actgtctcca gtcctactgt gataagtgtg |
| 33901 | agaactgagg tgtctataga attttgtttg acttgagagt tgaaaatgct tacgtctgaa |
| 33961 | taaggtgaaa gtgagtttta ttgggaagga ttgtaggtac aatcatgtta aattcatttc |
| 34021 | caggctgaat aaaacaattg ctattttgtt catgaaatag tagcacttca cacagtgtta |
| 34081 | acacctgcat accccaggtt caccttcatg actcttaata atctctattt cctgcaagct |
| 34141 | tagccctgca cagatattga atatggtttt gtgactggag acatggaagc agtgtttttt |
| 34201 | tttttttttt tcaagtcttc tatgatggac ttatcagttg ggtgccagtt gagtaacagg |
| 34261 | ttgttatact tattacatta ctacaatata caatcattgt gtttaataat tttgcatgct |
| 34321 | tcctatgtct ttctgtaact actctgagaa tggaaaccag tcattccctt cttatttatt |
| 34381 | ttctacaagt gccttttcag gtctagctct tagttatatg gccttgcaag cttcttctac |
| 34441 | tcaacactga gaaccactct cccctcaccc cacccagcaa ctcccgaatc ttgaggcttt |
| 34501 | acctcactaa accctgaaac taggatttaa tctatactca acttgatgtt gtcacctaaa |
| 34561 | cgcaacctcc caccccctta tattccaagt atttacgtcc aacactttcc agaagagaaa |
| 34621 | agggaaagga ccaatgtgta catgggaaga agctatttcc tcctttcttc tattgaaact |
| 34681 | gaaatctagt cttagctcta cccactccct cttttcttaa gaaattttat attatcaatt |
| 34741 | acaccaggtt aataaaatgg atttttctta ccaaaatttt gaaattccca ttcttatgac |
| 34801 | tgttgatatt ttattgactt gaaagaaata acttcatatt tattttaaac tacacaattc |
| 34861 | agcactctac tgtttgtgat cccatgggca aataagtggt gctctcaaac atttcattga |
| 34921 | agagttaaat agaggtatta ttgacgaaga tgtaaatagt gttaaaggaa tcaacaagga |
| 34981 | gtgttgaatc accagaacct acctagatga cttacatgaa gactgataag gcatgggagg |
| 35041 | aagcattatt actagacaaa gcaatagagc catgggagag agaccatcac tcatagtagt |
| 35101 | agtgagctac agtgcagaaa aagaaagaga atcactgcca ataccaaaat caggctatga |
| 35161 | ggaactggga ataagggata cactgatctc actcctctta tctatctctt ttcatggtct |
| 35221 | ccaactgcaa atccaaccag aagtcagagg ttatgggagc ctaagtaagg aagaccatga |
| 35281 | aagttagtct ttcaggacac agaggaaggc agtgaagccc tcagtgaatc cagaagaaaa |
| 35341 | gtagagaaaa accaacacga gcatctacca tttgatagga cctattataa gaaggttata |
| 35401 | tatattaact tatttaatcc ttataaaatt tcatatgagt ggcactaatt acattataag |
| 35461 | gataaaaata gggtacataa gagttaacta aatttctgcc tccatatagc tagcaggagc |
| 35521 | cagacataaa tttcaagatc agagatcctg gttccacaaa ccatgtcctt agccactctg |
| 35581 | ataatctKcc ttctgaatca gaatggactc atgttaggta taaatgaaat ttgatataat |
| 35641 | tattgagatg tgaggattgc atatgttgga ttgataactt aggagaattt tcctaaattt |
| 35701 | cttcatatat agatttcttt gtattcaacc atcaccaaag ccacagaaat atgtcttaga |
| 35761 | aatggaggaa aagaaagaat gataataaaa gcagtctcta ctacttggtg tctcttctaa |
| 35821 | tctctttgtt tccataattt gcccactgat ctttacttct taccaagtgg tccaagtgta |
| 35881 | ttactacaaa ggaattatga agattttccc gtttttaact tcctgagtta tgcaatgtag |
| 35941 | atagcaataa atgaccaatt aagtcattca attgttttca tggtaattca tgaaaacact |
| 36001 | attggaaaca ttactgagtt ataaacataa tcataatata tatgataata aaatggaaag |
| 36061 | gaattggtta caaattaaaa gaaaggcgtg cgaagctatg ctgcaacaag tataagatat |
| 36121 | ctgagaacca taaattggca tttgggagga gctcactgat tagtaattct tatttaccta |
| 36181 | ctttgattgg ccttaaaaaa gacatataca ttcctaataa acatacattc actcatgaat |
| 36241 | tcacacaaaa cagtcaaaga tcctgacttt agagagtttt tttgttgttt gtttttattg |
| 36301 | agacaggctc tcactccttc acccaggctg gagtacagtg gcatgatcat ggctcactgg |
| 36361 | agcttcaaac tcctgggctc aagaaatcct cccacctcag cctcctgagt agctgggact |
| 36421 | acaggtgcat gccaccgtgc ctgactaatt tatttttcgt agagacaggg gctcgctatg |
| 36481 | ttgcccaagc tggtcttgaa ctcctggcct caagtaatcc tcccaaagta ttgggattac |
| 36541 | aggtatgagc caccgcaccc atccagggag tttgattctt gcaagatatc ttctaaagct |
| 36601 | ctcaaaatag tcttgaaaaa ctgcagtgca gttgttctcc attaaataca caatagagca |
| 36661 | tttttttgcc tagaattacc ataaaaataa ttcaatatat aaattgaatt catatataaa |
| 36721 | tatatgactg gaagtctata gtaaagactg tgatagtgat ttttctgcat tttataaatt |
| 36781 | aaaaaacata gtttttcata ttttaatctc tctaaaatta ggatgcatcc ttcaacttct |
| 36841 | atttgcaatt gtaattagca ccatgtttgt ctttattagt gatgtgtaaa atagttgcac |
| 36901 | atcttacaat aggaaacatt ttagattaaa taatgtatgg tatttttaca aattaagtta |
| 36961 | atggatattc ctggaaagtt aagttaaacg tcactgggca agaagccaca taaagagaat |
| 37021 | aactatttta aagaatacct ccaaaagaaa aggcagtagg aaatatacca attagaaaac |
| 37081 | ataggacagt aatagcaatg agaaaaatta ctgtctaaat taaaactgtt gcctcaatca |
| 37141 | tagaaggata tacacagaaa taggaaatac acagagatag cttatcttga atcattacca |
| 37201 | caaagcaata tccaaaatat ctattgagag aatggtaggt ttttctaagc aactcaccaa |
| 37261 | aggaaggatt gtttgcctgg ctgcagcaga cagccaccta ctgagttatc attttgagta |
| 37321 | atttcttttt cctaaaaaat atccatcatt agtctcactt tttggaattt cttttttttt |
| 37381 | ttttatggag attgtgactg atatatgggc accagagtct taatttgtac aaaaacaaaa |
| 37441 | aaaaagtatt taggttagat ttagttattt atatacatca gcatgaagta aagcaatgta |
| 37501 | cttaatttcc tcttaatata ccttttaaca aaggaataaa taatatataa ttcatttttt |
| 37561 | attcaaagta cttcacacaa ttttgttgcc tttatttcac caagtctcct ctatttaata |
| 37621 | cacctatgct aagaagcttt ggcacacact gcaaaaagca ctSaacttgc agtcaaaagg |
| 37661 | tatgactttt agtatcgtac tagtttttct gcttaattga tctttgaatt tatggaaacc |
| 37741 | aagacgctgt tctgaacttc aatttttcca tctgtaaaac aggaataata aaaccaatca |
| 37801 | caaagggtta ctactatatt aactggatga aactatgtga aagcaggtag caaaatgaac |
| 37861 | agtgYttcta catacattat ctctgcagta aaactatgat gtgtaaactc agtaaagaaa |
| 37921 | aaaaagcatg agtgatcata aaagtctaat tattcattgt catcaaactt cagatttttg |
| 37981 | tgggagattg gaggactcta agggagacaa acaaatgtaa actgcagaat aatgagtttt |
| 38041 | aggccccgtc actgttacct caatttgaaa acctgctaat gaggaccaca ggaactcttc |
| 38101 | tgccagtgaa gctggtactt cctagaggaa aaatgcacaa aaaacaccta ccaccgaaaa |
| 38161 | caaaagcacc ctgaattatg attttcaaaa atatctagaa gtttatatat caattatWtt |
| 38221 | atctcatata aactgtgttt gattccaggc tagctttaaa tagactctat attttaatat |
| 38281 | ataactaaac aaatgcctcc agtgatagtt ctgaaacgtc attgtcctta aattttttgc |
| 38341 | taaggacatt gtccaagtgt ttgctactta gagactttca gaattttcat cgctacctca |
| 38401 | aagttcaagc ttttctgagg tcccagcgtt tgagcctctt gaacctggag gcaaggttag |
| 38461 | gggttgggaa agtaaacttg tcaagagact ctactgggca agccctcaca gcaaggagtt |
| 38521 | gggaagagag ctgacagcaa gctctggtgg ctcccacagc aatgtcttac gagggctcct |
| 38581 | tcggaggagt taataaataa taaagtttgg ttcaggactc caggcaagaa tgacaactct |
| 38641 | ggctgaggaa atttccaggg gctgatatct aaaataaact taaagtcaca ataaaatatg |
| 38701 | ataagtgcca tgtaataatc ctacaatgga aatacacagg atagcattgt ggttaagaac |
| 38761 | acagttccca cagtttcact tctggggtgg caaatgagga gttccatgga cccactcccc |
| 38821 | agcaaaataa gcagagatgt caaaaatgaa aaagaaatga acaaaaaaca acactaacaa |
| 38881 | aaaagtttac attctctgga aataattgca aatcatatat ctcataagtg atatttagca |
| 38941 | tatacaaaaa tattataact cagtatgaag aaatataaac aaaattaaaa aaaattaaaa |
| 39001 | tgggcaaaga tctgaacagt tctgcaatga agatatattt atataaatgg tcagtaagca |
| 39061 | aatgaagaga tgctcagcat agttttccat caaggaaatg caaatcaaaa ccacaatgag |
| 39121 | ctatgactac aagcccacta ggatggctat attcaaaatg acagataata acaaatgttg |
| 39181 | gcaaggatgt tgaaaaatca gaatgctgat acattgctgg taggaatgta aaatggtacg |
| 39241 | actccttcag aaaacagtct agcagttcct caaatggtta aaagtagagt cactacatga |
| 39301 | ttacaaattc cactgtatat ataatcccaa gagaaatgaa aacatatgtc catatgaaaa |
| 39361 | cttgtactca aatgYtcata gcagcatggt tgattgcagc caaaaatgga aacaacccaa |
| 39421 | aggtctatca actggtgaat ggatacataa aatgtagtat accataaaat ggaatattat |
| 39481 | ttggcaatag aatgaaatta catgttgata acagtgtgca gcatggatga accttgaaag |
| 39541 | catgctaagg gaaagaagYc agacacagag gatcatgtat tatatgaatt catttctgtg |
| 39601 | aaatgttcag aatagacaaa tctataggga ctcacagggg caaaaagtaa ttcgtgattg |
| 39661 | cctaggtctg ggaaagtttt ggagattggc agatgacagc taacaagagt ggcatttttg |
| 39721 | agggggagct ggtaataaag ttctaaaact gactgcaatg atgattgcac atatttgcga |
| 39781 | atatactaaa gctattggat tgtgcacttt ggatgggtga attgtatatt atRtaaatta |
| 39841 | tatctgaata aagctacttt ttaaaaaaga gagagtctgg ttccccagac tgcctatgtt |
| 39901 | taaatcccca cctctcctac ctgtgaatgt aggccaggta tttaaactct atgattctgt |
| 39961 | ttcttcatct gtacaatgga gataatataa attacctacc tcataaagct gttataaaaa |
| 40021 | ttcaaataac tagtacatgc aaagaacaaa gaccttctaa cagaacctgg cacatgaaaa |
| 40081 | gtgtgatatg tgtccattct tcttatgcac agactgttaa gggagagcat gaaaRaagta |
| 40141 | tccaacttat gggtaaggca gtcggcaagg cttaaaggat gatatttaaa ttcatcttga |
| 40201 | aagatgagca gggattatca aggaatagat aagagggaag ggatatccag cattagaaag |
| 40261 | agtatatgcg gaagtatgta gatatgatat tctatcaaga atttaaagta tcctaaaagt |
| 40321 | aaattcaaat attttctgaa gtatatgaaa ttgaaataca gaaaattcca gcaacctatg |
| 40381 | tgtcatgtct tctctttttt aagagttcct ttagagacaa ttcagtaaga aatttctctg |
| 40441 | tgactatgaa ggtaatattg tatgatgtaa ataatattca ccaagttaaa aataacattg |
| 40501 | ctacaactct gatccagtgt aaagtttagg taaaatagta ctcattttta ttattgaaac |
| 40561 | agtgtaatca gtagacatag ctctgtgttg tgtacatatt ttgttttgtc tcttcttctg |
| 40621 | agtgtactca tcttaggtga atggggaaaa tatgattttt ataaacagac tcttaaagaa |
| 40681 | aaatggccta tgcaaatgat gtaatttaga gcaaccatag tatatgtgtt cacttcagcg |
| 40741 | ctcactaaat aaaatcacca taagaattga tgcatgacaa tggcttgaca atttcttatt |
| 40801 | cttttgtatc acaccaattc ttgaaatgat agctgttgca cagacatgtg tgagtattgc |
| 40861 | tattgtatat gtggtagcca tgcatgaccc tacttttctc ctcttggaag ctggaacctg |
| 40921 | aattcttaga ctaatcaatg agtaaatatt tcagagatgt gttatctaat ttacttctga |
| 40981 | aaaacagatc tactacttgt ctctagacat ttagaaagag attgacagag tcatttgcaa |
| 41041 | attcaaagat ataaaaacag taggtagatt gacttccata atcaaatcaa tatttggtta |
| 41101 | gaacatttca caatattcta accgaaatta catgaactac attatatact ttcagaaatg |
| 41161 | cagtgtacca ctccatctga acaaaaacat aatgttaagg tgtccttgaa gctttaagtc |
| 41221 | atcttttggc aatgcagtag tttcaacata atcacaagac aaaaccagtt ccacataaaa |
| 41281 | ttcttcagaa atgaacatga atataaataa aaacttgaga gatgctactg aaatagattt |
| 41341 | tacatttcag agttacaaag gaacacatta gtagctatta aagcttacag ataacattac |
| 41401 | catctagctt actaataaat ctcattgtta gtattttctc tagtctttac tgaaccttta |
| 41461 | agattattaa tttttttcat attttattga tatggctagg atagagttat taaataactt |
| 41521 | cttcaagtta caaatatgct aaaaataagc attgaatcct tatctatcta atttactccc |
| 41581 | agccatgatg attctaacaa ggactggcct aatttcctgc cacaaaaggc tataaaactg |
| 41641 | agccaagtat ggaaaaacct tttcagacat tagtcaattg gtagaaatgg actttgaYac |
| 41701 | ctaaaaggtt ggaaacaact aaatgatccc aacaattatc ccagctttct tgctgctggc |
| 41761 | agtttccata ctggagacat gaaagtatgg atccaaaaca gaacttgtca gcctcactga |
| 41821 | gtttacgaag tagagatcag agggtgagaa agctgatgtg gttggcgcat attatctcat |
| 41881 | ttttgagata tgttgtaaat tagagggagc tgagcaggaa aatatttaca gaaatgtgaa |
| 41941 | tcttgcctga gtactagacc atcaatgcta gagagaaatt ctacaagtgt gggcaaagaa |
| 42001 | tgacaggaat cagaacaatc gccagagctc acacatgatt tttgagaaag gtttcatttg |
| 42061 | gggagatcgt attgtatcaa gaattcaaaa gaaaatgtag aaagtatgta tttatctttg |
| 42121 | agttgtgtag tgatcgatag aaatttaggt gaatatctaa atatctatat agatataaag |
| 42181 | taggatactc ccacacacct tgctatttct gtgttttaca tatttccctg acctcccaaa |
| 42241 | atgtccagYt ttttgttagg aagctgcacg ctcatcttcc ccttctgagc tccaagagca |
| 42301 | tgttgatgtt cttgctgttt catagctgat ataaattagc attgggtaaa cgacgaaaaa |
| 42361 | acgtcacaga aagtttttct tgttcacatt ttggaaatct tccttctcag tagagaaaag |
| 42421 | caaccttaaa gttgcaacat catgattgga tttcagttca caaaagaaaa taaattttga |
| 42481 | caaatcacat atgcttttcc tgtccctccc aaaatgctgc agtcttccat ggcagcctta |
| 42541 | ccaaagaaaa aactaagtcc ctaactggaa Sttggattct ttcaaaggca cagtaagagg |
| 42601 | ttttgaccaa tagagaagca ttgatcaaat cactatccac tgcattacat ggtactctag |
| 42661 | gagagtctga aatgagaata gatatcttca aaaagcatcc aaatctgggg ctggtttact |
| 42721 | catctgtcag cttctggaat agtgaacatg ctattaaaat tcagctcctt ctgggaggtg |
| 42781 | ggaagataat caaggggaga cccaccatac tgcatggaag tcccagaaat ggaaaagctg |
| 42841 | atcaaaggaa ggaaggtaat atactttttt ataatatgtt ccttgggatt tttaatgatc |
| 42901 | ggtcgaaata cagaggtttt ggttttaagg tgttattttc acagcatctc caggtaaact |
| 42961 | tccaagtatt atgttaRcct ggatagaaac aaaaaagtcc ataatgcttg aaactgaata |
| 43021 | tgtgatcctt gcatcatgtt taaaattgtt cagcaaggaa tttcagtttc tctaggtgat |
| 43081 | ggaagttagc acgagcaaag gaactttaca atttttcaga attttttttt tttcatgttt |
| 43141 | ggaaggtctc ctcctcctcc tcctccttct cctcctcctc ctcctcctcc tcctcctcct |
| 43201 | cgtcctcctc ctcctcctcc tcatcctcct cgtcctcctc ctcgtcctcc tcctcttcct |
| 43261 | cctcctcctt ctcctcctcc tcctcctcct ccacctcctg cttctccttc tcctccttct |
| 43321 | tcttcatttt tatttttaac cattgctagt ggtatgattt ttgaattggg aaaacaaaat |
| 43381 | attcagacta aatattttcc caatgattct tatgtagagt aatgtacttt gcaattcaat |
| 43441 | taaaaataac aagctaggat ggtgttctga gccaaatgta atctccaaga ccatggtcag |
| 43501 | taaagtactc aagaaaaaag tattgaggcc gagtagtctg aagtctaYtg gggaataaag |
| 43561 | aagcagctga ttagcatttg aaagcaaggt ctttcactgt tttaaaagtg aaggagaatt |
| 43621 | ttttaacaga Rcactcatca aaaacaaagc gtttgaaaat tacccctcct ttgttttatc |
| 43681 | agtctcaggg aagatgcact tcgaYacacg tctcaggaat taagatgcat ttcgatacat |
| 43741 | gtaatattac atttggtgac actgctggaa catatgctga gaaatgcaag aataaatagg |
| 43801 | actaatgctc attttcNttt tttttttaat cacttgcaag tgacatttgt ttcatctagg |
| 43861 | caggaaagca ggatcttata ggcagggtag gtgtaagggc tgattttagg tcaagctttt |
| 43921 | caatttatta tgcaataaaa tatagcaata aaaatatgca tctttatatt ttccagtaaa |
| 43981 | tbataggaat ctccgtttaa ttttttatat atgtgctaat aattaattca gtactacagt |
| 44041 | ctggatccac agtccttttg ataagagctg gcacagaggt catttttatg tgccttatta |
| 44101 | ttaatcttaa taatatgtag taatattcct ttatatgctt attgtgacac acagaaactt |
| 44161 | aaagcctttg tagctttaag ttcagatgtc tccacattct tttgtaaatc cctgcagatt |
| 44221 | tgtttcttgc ttttaaatat cagcaccttt ctgctgtcta ctcttaggtc tctgccctct |
| 44281 | atgcaggaga tcttttcttt taagtttccc accgtgcttc ataataaaat caaaagaaaa |
| 44341 | aggtacatcc ctcagaaagc attcatctca ctaKaaatag aaaacctgtc accttattct |
| 44401 | taaaacctta gatcagccag gccagtggct gaaatagtga gggtagtgtc tacaaagggt |
| 44461 | ataKagtagg ggtagcaaaa taattatgta ccttgttctc tccacatctt ttagggaaga |
| 44521 | gactctttgc tctggaaaat gacttaatac cattgggagg aaaacatttc ctctacccac |
| 44581 | ttaggttcct atgtttaggg gcctgtgaat taactgataa tagacagatt aacaggagaa |
| 44641 | aagaaaaggt taatctacac atgcatgtga agtcactcaa taatgagtcg tttcctgaat |
| 44701 | acccagaggt aaagattggt atagcaactt aaaaaaggga agggaagtgt ggctagggca |
| 44761 | tcaatgtgta gttgatctcc ttcctggRat tagcagaaag ctcttggagg agagttaata |
| 44821 | gcagctgtat tgggggtagt gtcagcttta gttaggtaag ggaaagtcag atcagatttc |
| 44881 | aaatattttc actttaatct ttatgctaaa actgtgattc cttcaatatg ctgatgaaag |
| 44941 | ttgaagtgtg gcttggaaaa aaagagaaag tataagtaga tttattctag atataaagca |
| 45001 | ttttttaggt atagtagaaa gagagaaaga gaaagcaaca ggcacacaca cactgagaga |
| 45061 | gagattttga cacgaccatg tcaaagacta agagtacccc aagttcttta tcaaccatgt |
| 45121 | agtttctgca acctgcagta tacacacatg catacccata tactgatgga ggcagtctaa |
| 45181 | attaatggtt aagaatatga acttcagcac cagagggcat tggattcaaa tttcaatctt |
| 45241 | tcttactagc tttgtaactt caaacttttt tcctcttgga agctggaaac tgcattttta |
| 45301 | gattaaacaa tgagtaaata tttcagagat gtgttataca gtttacttct gaaaacagat |
| 45361 | ttattacttg tctctagaag tttagagaga gatcgacaga gtcatttgca aattcaaaga |
| 45421 | tataaaaaac aggtagactg actaccataa tcaaatcaat attggtttag aaaatttcac |
| 45481 | aatattctaa ctcaagttat gtgaactata ttatatactt ttacattatt tatctaccct |
| 45541 | caagtttcag ttctctcctt tgcaaatgac aataacaata ccaaaaaata aaaaaactaa |
| 45601 | atgaggtaat gaacgtaaag tgataaggat gatgacaggc ataaagtaag ctttttattg |
| 45661 | tttttatttg ctttttcctg tagcctgaaa atatgttcaa ggtaaaaaaa agcattcttt |
| 45721 | tgctctgaat ctcctttatt ctaccaattc ttttctcgtg taagtgtctc agactcttca |
| 45781 | gttgtcttca ctttgtcatt tatttttatc ctcaaatcat tgatatttgg cttctatttt |
| 45841 | cacttctcca aacaaattgc ttttttgtct tcatgatctt ctacgtgaca aattaaatgc |
| 45901 | atgctttcag accttacctt cctggacctc tcacctccat tggaatccac tggcaattta |
| 45961 | ctcccagaag ccctgtcctc tcttgcatct tcaacatctt ctgattctga cctctctatt |
| 46021 | cctttgttta tttgtttgtt tgtttgagac agagtttcac tcttgttgcc ccaaatggag |
| 46081 | agcaatggca tgatctaggc tcactgcaac ctccatctcc tgggttcaag tgattctcct |
| 46141 | gcctcagtct cccaagtagc tgggattaca ggcatgcgcc aacatgcctg gctaattttg |
| 46201 | tatttttagt agagacaggg ttttgccatg ttggtcaggc tgatctcgaa ctcctgacct |
| 46261 | caggtgatcc tcccgccttg gcctcccaaa gtgctgggat aacaggcgtg agccacagtg |
| 46321 | cccggcctct ctattccttt tacctccttc attggattct cttcttccag agtttcatcc |
| 46381 | ctggccttca ctatacttta ttgaatgact tcatttactt tctagaaagt tataaacaaa |
| 46441 | tttttatttc caaccctagc ctctctctcc tgcttcagac tcattatcca cgagtactat |
| 46501 | tgaaaaaagt attctatatt taatgacaaa aaaacagaaa atatggaaaa caaaaattaa |
| 46561 | cagcaaaatc ataaataatt cttccatcaa aagttacttg ctaatatttt agtgtctacc |
| 46621 | actagctaaa cttaactaca tttccttatc tataaaattt gaggtaattg aggtcccaaa |
| 46681 | ctcataaaac tattgagcat gaatacacaa aaagttttta aagtacccag cacatactaa |
| 46741 | gttccatatg aatatttgct gctattattt ttttcaattt caaacatgta cacacatgtt |
| 46801 | tcaaattcaa aaatgtagct ttgtacattt tcttcccatt tagcaatatg caaatgagta |
| 46861 | tttttaattg aatagatttt tttttttttg agacggagtc ttactctatc actctgtcac |
| 46921 | ataggctgga gtgcaatgcc gtggtcctag ctcactgcaa cctccgcctc ccgggttcaa |
| 46981 | gcgattctcc cgcctcagcc tccagagtag ctgggactac aggtgcatgc cactacacct |
| 47041 | ggctaatttt tgtagtttta gtagagacga gtttcactat gttgtccagg ctggtcttga |
| 47101 | actcctgacc tcgtgatctg cctgcctcgg cctcccaaag tgctgggatt acaggcatga |
| 47161 | gccaccgtga ccagctgtga atagatattt ttaacagatg tgatcagtag attttttcag |
| 47221 | ccacttagtg aataatttct acagctgcct caaatgtcta ttaatttatc tagggttgta |
| 47281 | tgtaattccc agccttctaa ctgttgacag ttttatttct cattccctga actatgttga |
| 47341 | aatgcaaatt aatgtcttta cagagatatt tttgaatctc tctattaaaa aaaaaaaaag |
| 47401 | aaaaaagaaa aaaacaaacc aatcccgaac ttcaaaatgc tattcttggt tttttaaaaa |
| 47461 | atgacctata gtccaactct gtgtagataa ttatgtgaca gggcattgag acactgaagt |
| 47521 | tgacagccca atgtgctgtg ctgtgtcata cagatagact taaaaaaaaa aagccccagg |
| 47581 | ctgggttctg tggctcatgc ctataactcc agcatttggg gatcacttga ggtcaagact |
| 47641 | ttgagacaag cctgggcagc atagtaagac ccatcccccc cccccaaaaa aaaataccag |
| 47701 | gcataacagt gtgtgcctgt attcccagct actcaagagt ttgaagcagg attgcttgag |
| 47761 | cccaggaggt tgaggatgta atgagccgtg attgtgccat tgcacttcaa cctgggcaag |
| 47821 | ataacaagac cccatatctt aaagcaaaac aaaacccatc tctgattgta gcgtatttag |
| 47881 | gttgttttca acttcttcct agataacttg caatgaacgt tttaaaaata tatacttgtg |
| 47941 | cacatttatg ctcatttatg attatttaga tgtacaatta ttagatgaga taatataaaa |
| 48001 | aatttacagc ctttcacttt tatttttctt tttcttttat tttactttaa gttccaggat |
| 48061 | acatgtgtag aatgcgcagg tttgttacat aggtatacat gtgccatgtt agtttgctgc |
| 48121 | acctaccaac ccgtcatcta gggtttaagc accacatgca ttaggtattt gtcttaatgc |
| 48181 | tctccctccc cttgcctacc ccactaacag gccccagtgt gtgatgttcc cctccctgtg |
| 48241 | tccatgtgtt cccattgttc aactcccact tatgagtgag aacatgaagt gtttggttgt |
| 48301 | ctgttcattt gttagtttgc tgagaatgat ggcttccagc ttcatccatg tccctgcaaa |
| 48361 | ggacatgatc tcattctttg ctatggctgc acagtattcc acgatgtgta agtgccacat |
| 48421 | tttctttatt cagtatatca ttgatgggca tttgggttgg ttccaagtct ttgctattgt |
| 48481 | aaatagtgct gcagtaaaca tctgtgtgta tgtgtcttta tagtagaatg atttataatc |
| 48541 | ctatgggtaa atacccagta atgagattgc tgggtcaaat ggtatttctg gttctagatc |
| 48601 | cttgaggaat cgccacactg ccttccacaa tggttgaact aatttacact cccaccaaca |
| 48661 | gtgtaaaagc gttcctattt cttcacagcc ttgccagcat ctgctgtttc ccagcttttt |
| 48721 | agtaatcacc attctgactg gtgtgagata atatctcatt gtggttttga ttcgcattgc |
| 48781 | tctaatgatc agtgaatgtt gagcttcttt tcatatgttt attggtcaca tatatgtttt |
| 48841 | ctttatagaa gtgtctgttc atatcctttg cccacttttt tatggggttg aggctttcac |
| 48901 | ttttttattg tgaatttgtt ttctggaaaa cttttaccaa ttttaattca cataagtagt |
| 48961 | gYatgagaaa gtctactttc tcacatgctt gcKgataaat gttaacattc atttttatcc |
| 49021 | ttgccagtca gataagcaat aaattaaata gttcttgtta ttttaatttg tattccccaa |
| 49081 | aggtatgtta agagagcaaa acaataaatR attcaccctc aatggttgct gttaaacttt |
| 49141 | gtctttttaa aatttaagct ttaaagatta tgactttaat tattttttct tctcataaat |
| 49201 | aatatttctc ttctaaataa ttctaagtca caattatgta gggataaaaa agaaagtata |
| 49261 | cattttaatg ctttcctcca ttaaatttga atgtgtgtta attttgtatt cagtgctgca |
| 49321 | ggtttttaca gaatactgtc tttggttcca tattgaacat tcaccttgag gactttagtc |
| 49381 | tattgattga aagctttagt tcatctaata gaattattat gacacacact ccaRctaaaa |
| 49441 | atcagtcaag gacagtccca gaagttaata ctcttccaaa gactgatgcc tccactgagc |
| 49501 | acctctagga agtatgagat ggKgttgcat cacttctcac taagttctta taaccccaca |
| 49561 | tccaatatat caacaaattt catcagctct accttcaaaa attacctaga atccaattat |
| 49621 | gtcttattaa atccatttta ctgccaagtc acttctgatt tattgcaaaa ggctcctaat |
| 49681 | tgatctccca gcttcttcac ttgcctcttc cagtctattg tcaatagtaa ctgtcaactg |
| 49741 | tStagtaaac tgtgaaaata tatctgatca tgtctctcct ctgctcagag cccatctggc |
| 49801 | ttcctatctt agtcaaagta acagctgaca tcatcatagt ggcctaaaga ctctagtgaa |
| 49861 | gctctgcccc atccaaatac attgcctctc agatatttac taatatStgg ctctagcttc |
| 49921 | cttggtccta gccacgctag tgtctcttct gtctcttaaa caagtcagag actcttaact |
| 49981 | gctgtattta tacttactct ttcttctgtc tgaatgctgt ttctacaSag gtgctttcta |
| 50041 | attgaaggta caatcatttc tcacatatgc cttcttctca ttatttgtKt aatttctctt |
| 50101 | cacagcacct atcatcacca tctgacattt tatatatata tatatatata tttccttttt |
| 50161 | tgtttgttgt ctgtcactcc cactaaaatg taggtctaga agaatagaaa tgttcgtttg |
| 50221 | ttgtttgttt tgttcactgc tgtattctta gtgtgtgcct ggcattaaca ctcttttgaa |
| 50281 | tgactgaata aatatctgaa tttctgctat ttccctataa gtaatattat tttggataga |
| 50341 | aatgccacta atcctgaaac attttcaagt tttatatggt ttttatcttt aatatttgtg |
| 50401 | tggctttctg ctacattcca atatttaatt agctttagtt tacatttatt aaatgcttct |
| 50461 | atatccagat aatgtcatat cataagtttc agttgacatt tattaacatt actacttctt |
| 50521 | ataatatttt aacagaaata tgattcatca actccagtgt ttgtaaatat Oatcaccatt |
| 50581 | ctgaagtcaa atgttgccaa agtcttccat taaatacttg cagacttctc tcatttggta |
| 50641 | cagccagtca ctccttacag ctgcaatatg ttatatctcg aatgtatttc ttgatagctt |
| 50701 | tctttaagat aactgcactt gcaaagttct ttgtacctag aaaactttca acaaatattt |
| 50761 | agagaatata atttactcat gcatgtggat agacacatgt aagattcact taagtgtcaa |
| 50821 | atttactaat ataatgaaaa cttggaccaa acaagcaaca aaaaatatta tccagcactc |
| 50881 | atactcaaaa cgcagtctga gagtttggta tttttcagtt cctaaaggtt gatcatgtgg |
| 50941 | ttcataaaat cttagtctcc aaaatgtatt ttcaaatgaa tgtcaggcag aaccaacaaa |
| 51001 | tgagaactag ggtacttagg attattgtaa ttgcctaatg tgtttggtga ccttgtattc |
| 51061 | ccacttacag caacttatgc ttctttcaaa ttcttttgta caaaaaaaat tgtcttgtcc |
| 51121 | actctccact tagcctttaa gtcactaaga tgtggctatt tcattagttg aaagcaatgt |
| 51181 | ctaaaataaa atattttatt ttaaaaatca atcatcttgc ataataatag ctttaacttt |
| 51241 | atgctcaatt taaaatattt gtatttaaat aattgcttac agaatagaaa tagaaatgat |
| 51301 | atataagata atatagaaga tgtaaggatt taagaagtta ggatgacagt atttttattt |
| 51361 | acttttatca tttgtaattg tatcttattg tctggtaaaa acatttaagg aaagattaaa |
| 51421 | atctaagttt aatgtattat ttataaacat ttgctccttc ataaaatctt cctgcaatcc |
| 51481 | ctctttttaa aaatgttttc attgcatatc ccgatgatct gcttttcttc tctccatgaa |
| 51541 | actcagatac tagttttctt tacaacacct ggattgtttc ttcctctMct gctgttcctt |
| 51601 | attttgtttt tctattctgg tgtcttgata tctcctttta acaaattcca taccattggc |
| 51661 | cttcctaact tgtgaacatt tctaatactc acatatgctc tataaaaaaa agtgaggttt |
| 51721 | ttccaagaag aacagtggcc attaattctg catatatcaa aatggaatgt gtcatctttg |
| 51781 | agaaaaatat aagctcacac catgcataaa gacaactatt gtttgcttca gccaggagta |
| 51841 | tgttttaggg aaaaaatatt ttaaacaagc cattaagaaa tagtctcttt tttctttaat |
| 51901 | gtctttgagc tacatatgtg agcagaatta taaagaattt ccagtgactc attcttagtt |
| 51961 | ttggccttcc tggcattctg tagtcgtgca tattaaaacc tctaaacagc tctcagtaaa |
| 52021 | gccttggcag tagagcttgt agcttgtacc tatcaaaaat gtcagatgct tttttccaac |
| 52081 | gccatcacta gcaaacaata ttacttagtt attaacatat aggaattaac ggcaaaatct |
| 52141 | gcaagttgca aacagtgttg taaatcaact aagcatttat catgagattt tttaagtgac |
| 52201 | tgaatacaga ttcagaatca tgaagttatt atctaataac ccaacaagtg atgtgaataa |
| 52261 | atccttgcta atatgaaaat tatcagccac tgaaacaccc ttttctacaa gcaggtctgt |
| 52321 | ggatcctcat gctaatgtac ttagattgat aaaccacaga cattctctga aagcttcgtt |
| 52381 | tgttccagtt gctgaggctt gtgcttagag catctgtcat gcaaatgatc agcaaatcat |
| 52441 | aacctgtaat ttgttttcat ttaataggca gacaggtttt caccaattta gactcaagtc |
| 52501 | ctattaatac tctttgaaac taattgctgg aaaatgaatt ttaatgacag gattttatca |
| 52561 | tttattattt tcccttctgt ccctataaag gaagactcaa agccatctac tcacactttg |
| 52621 | aaaatatatg ttaaataggc acaagccata atctcctgtc atctgggctg ttgcaaagga |
| 52681 | cccctaatcc cctctcttgt aataatccat tctacactct actcttaaaa ttactttctt |
| 52741 | actatcccat ttcccttcca caaaatcttc aattccctgt caagtaaata gcagacactt |
| 52801 | taatatggct tcgttgccct ccattatctg tttgcagttt acatattttt atccttattt |
| 52861 | gatatcgcaa tataacatgt atacagaaaa aagcatgaMa cattaattaa taagtagtaa |
| 52921 | agtaacatcc agaaaacact actcagagca aaaagcagac gattgtcatt accacagaaa |
| 52981 | ccacctcccc agtgcatctt tctgatcaga cccttcccag catcccagag tctaaccacc |
| 53041 | cagagataat cacttcctct attttcttta gattgtctcc agctacgtgt gaattcttaa |
| 53101 | ataatatggt caatttaccc atttttgaat gttatgtaaa tggaatcaaa atctgctttc |
| 53161 | tttggctatt gcttttttca ctcaaattat tctcttaaac attcattcat tttagggtac |
| 53221 | agcttcagtt aacttcaatt ttaagctgtt tagtattcca ctgcatcaat gtaccacaat |
| 53281 | atatttatct tttccatgtt gatgggcact tggattttta gattgtttcc acatttgagc |
| 53341 | tattacagac aagggtgcta tgagcattct tggatgtata gtttggtgcc cagtattctt |
| 53401 | ttgtattcat acctaaggag tggaatttct aggacttagc catgcatatt catactagat |
| 53461 | aaatattgat gtaatacgta aaaccaagta atattaactt gtatataagt agctctgtat |
| 53521 | gctactagac aagtcaaaat atttttcaaa atggttgtaa atttatatat cgagcaggag |
| 53581 | tgaatgctat tcactcctac ttctatttcg acttctagaa ataggagcac ttctatttct |
| 53641 | ttatatgctt gacagtactt gggattatca gactacctca ctttctgttc ctcctctatt |
| 53701 | tattcttgct ttaggatttt ctatcctctt gtttattctt ccttcaccct ggatattaat |
| 53761 | tctgacctat tgcaattcat gaattctttc ttcatctgtt tctaatctat taatgactca |
| 53821 | gtcatatgtt aactttggta attgtctttt ctaattctca aatttgttta gttctttttc |
| 53881 | aactgcgtaa caacactcct ttgtagtttc cagtttcctg ccaattaaaa aagcattgcc |
| 53941 | cttcatttct tcaaacacaa taaacataat atttccaata tgcatctgaa aattctagta |
| 54001 | tctaaattct tgaggatcaa tttctgttcc ttgtttcttc tgcaggttct attattgaag |
| 54061 | aattattatg ttggtgcaaa agtaactKag gtttctacca ttacttttaa tggcaaacac |
| 54121 | cacaattact tttacactaa tctaatattt cttttcacca catttctttg atttttgttt |
| 54181 | ggacatcata ttcaaaaatt atttgaagga atcatgttac acctaaagta atattacctt |
| 54241 | tttctagaga ggattgtgtt tttctctgtc aggaatctgg agcctccatc agggtaccac |
| 54301 | cataaattgt gttaaaattt gagattaaac tatgcagtcc tggattttaa tccataggtg |
| 54361 | gaaaaaaata gagtttcaaa gaggtaagct tcaattaaga cagattatga tgcctccctc |
| 54421 | ttctgtaaca tattgtgtga ctttgagttg agattcttcc taagttttct gatctattaa |
| 54481 | aaaagatata gctggtaatc cccaaaatca tgattttcct tttttttttt tttttttttt |
| 54541 | tacagtttct actgtatttc tttgttgtcc tgtcagtacc agttatggtg tgtgttgctt |
| 54601 | tttctttctt tatatttttg ttgagcacag tgattgagta catgccccac taatagggta |
| 54661 | taattatttt gagagtaata tctatgactt aacaccatat ctctcactat attgagtgta |
| 54721 | gtaccttgat gcagtagcta gctgctcagt gaatgttaaa tggataaatc aatgactgaa |
| 54781 | taaattttag agataggtac atagataaaa gtgatcaaaa caagctaagt attactgcag |
| 54841 | agaaggaaca agtatctttt ctattatagt tcccaaagct tcaaccaaat ttcttttgct |
| 54901 | atgtttacct gtccaatata ttggctggtt gtactcatag agtactagtc ttagttagaa |
| 54961 | tttatgagta atggtccaaa tccgtaatag agcttaaaac taaagcttta aaaacattga |
| 55021 | tccagatgaa gaatgatagg atgcaatgaa agtttattta gggcataaaa tttaaaaggt |
| 55081 | atgttatcaa taattcattt tgaaagtccc cttaatatag ccatttctat tctgtatctt |
| 55141 | taaccagtta taggtgacat tgctaggaac agattttgac agcagacttg tgcaagggat |
| 55201 | ctgtttgcct agagatttac ttaaaaggta gaattaaagt catttcaaga aagaaattta |
| 55261 | acataagaaa atataaccat ttagtacact aaataattgg tctagctagt tatgagttaa |
| 55321 | aaatgaaaaa gaattaatag tttttagcaa catatacatt gggacagtat ttttgccaca |
| 55381 | tctatcattt acctatgcaa taattaaatt cataattttt taagtgtcat atcgaagagt |
| 55441 | gttattcaac ttcctggtga ttaaattact gataaacata gccgattttt tttggttttg |
| 55501 | caggtcctga aaaatagaga atgcatatga cttccaaata ttggtagaag agccagtcaa |
| 55561 | ataatatctt ccatgaatcc tcttacaatt aatttgctga ttgatagatg ttcaaaatct |
| 55621 | agaaatgaat gagttgcatg taaggaaatc agtacatgct tctgctgcca tcatatatca |
| 55681 | caatacaagc attaaaagct atattaatta tagaacttta tgatcttatt tacatggtct |
| 55741 | gtgaatttga tctttatttt ggaattcctt taatccattc tgattctaat cacctcagga |
| 55801 | ccaactttag tgtatttgta caaggcgtaa tcattggcta gctctataag ttgtccaaac |
| 55861 | aaccaaatga gatgtcgcaa attctgccaa ttgtgtaaag ttttattatg tactttaaat |
| 55921 | atatgaatat ttggaatatc aagatgtgtt gtattttacc aataatatta aaggtttgct |
| 55981 | tgttcacttt tcacttgtat tatgatgtca tttaaacaag tcagtattat tagaaatctg |
| 56041 | attcattctg aaaacctctc atctactgat ctaatgatct tcaatattca ttaacatgaa |
| 56101 | atattactat aataggaaaa aaatcggtac cttaacaaaa aatatggaaa tttatttgtt |
| 56161 | tatttattat ttttacttat ttatttattt atttatttat ttatttattt atgtttttga |
| 56221 | gacagtctca ctctgttgcc caggctggag tgcaatggtg tgatcttggc tcactgcaag |
| 56281 | ctctgcctcc caggttcacg ccattctccg gctgcagcct cccaagtagc tgggactaca |
| 56341 | ggcgcctgcc accacaccca gctacttttt tgtattttta gtagagacgg ggtttcacca |
| 56401 | tgttagccag gatggtctca atctcctgac ctcgtgatcc gcccacttcg gtctcccaaa |
| 56461 | gtgctgggat atttctttct ttctttctta cattctagag ccttggcgag ttggcccaag |
| 56521 | tgttgtacag cattttcRgt gtcagggaca tagacttctc tcctttctct gctgtgtgta |
| 56581 | gtctcagata gttaccagag caccagccat tgtatgttct ttctagtttt caggtagcag |
| 56641 | aaagaataag aaatgttgca caaaccaccc tgtttacatc acgttggcca gaattagtca |
| 56701 | catggccatg aagcaagcca ctttgcctga agtaggacta cagattgtct ttattctcca |
| 56761 | tagtggtaaa atataaaaaa atataattag agaagaagag aactcctctg gaggacaact |
| 56821 | agcagtaacc tactctacta actgtccatt tactagttca ttttgctcta agataacata |
| 56881 | taaacagtga aactaaaata tgttttcaaa attagaaaat gtaagtacct tagataacca |
| 56941 | agattaaaga aatgttacaa ataccacaat atcaacatag ttatagatga acatcaaact |
| 57001 | tagtactgat tttctgaata tccaaaatga aaagaaatac ctcaataatt ggttacattg |
| 57061 | ttttttatca ttttgaaaaa ggcactactg aggcaaatga agctctttga ccctgaacaa |
| 57121 | ttacaaaatt tatctcatag caatttttag ggagaaaatt gtatattata gttgacaata |
| 57181 | ttcctataac aggtgcagtg attttataag ctacttacag tgtctttgaa taaaagccaa |
| 57241 | aatgaaagca ttatgaaggc tattttgtga atctctgagg aaaaatcatt caaaaaaaaa |
| 57301 | aaaaaacctt tggctattct ggcttatata caaggagaaa ttttagcctg cccagagggc |
| 57361 | tggatggact acatacacca tgaatggtta tctgtaaata ttactgttgc tgaaagattt |
| 57421 | ctgaacaaat gtagaaaatt aaggaataga ttatctagtt ttctgtagtc tgccagcatg |
| 57481 | tttcaaatca atcgagcttt cttaacacca ggtgttctca gaagtgaaag gggcctattt |
| 57541 | gttagcttta aaaagacaaa aatgggaaaa gataaaagag gtggaagcag aaaccaattt |
| 57601 | gctaaattta ctaaattaga cagtttattg aaaaggtaag tgaatttctc atatcagtca |
| 57661 | taaagaagct atttttccaa tgaaatgtgt gtttatatca aagtcacaaa attacttagt |
| 57721 | gaataactcc cagtaaatgc gatattatta atttcagcta aattatatgt tcttagaatt |
| 57781 | tttttatctt ttcagattgt tttatgtaga ttatagctgt taaaatttta ttggatgtaa |
| 57841 | tttacatggt ttacaaacaa taaattcctg agaatagata cgataaattt atagtttgtt |
| 57901 | gaatgattta ttattaagga gtctcagatt ggtcctccga ggacaggaaa aataaattca |
| 57961 | tggaccctaa gttttttagt tacttttatc taatttttga tatggtaata attaaaaggg |
| 58021 | agatgataag aaattgaatg attaagcatt gttaaataat atgatgcgtt aataatgcat |
| 58081 | aagttatatt cctgagttga ttctttcaaa gtataaaaca atttttagat ttaatcaaaa |
| 58141 | ctctttaaag aattaatctt tgcttttagg gagcactata gattgacatc agaaatttct |
| 58201 | cttatttcca tgtaggaaga accttttgtg atggtctctg aaaatgtctt gggtaagccg |
| 58261 | aagaaatacc agggcttctc cattgatgtt ttggatgcct tatctaacta cctgggtttt |
| 58321 | aactacgaaa tttacgtagc accggatcac aaatacggaa gcccacaaga agatgggaca |
| 58381 | tggaatggct tggtaggaga acttgtcttt aaggtaagaa ttactttatt tatttgtctc |
| 58441 | atacttaaag gttcaattat ttgtctcata cctaaaggtt caacagtaac accttgaagc |
| 58501 | tgatttcata taaaataagt gtgatgtatt taggttggtt aagcattagg tggtggaatt |
| 58561 | gagtacaaaa gtctcactca atactatttt atgaactttg tggaaagaaa agtagttgac |
| 58621 | aaaataaacc taaatgttaa aatatatcct gtaatagaaa agggtttaga agaagacctt |
| 58681 | ttctttgctc aacttatacc ccaaaaacag aaagtgtata tgatacgtga tgaaatcaca |
| 58801 | tttttttttt gagacagagt ctcactctgt cctccaggct ggagtgcagt ggctggatct |
| 58861 | cggctcactg caagctccgc ctcctaggtt cgcgccattc tcctgcctca gcctcccgag |
| 58921 | tagctggact acaggcgccc gcccccacgc ccagctaatt tttttgtatt tttagtagag |
| 58981 | acggggtttc accatgttag ccaggatgga ctattgtttt ctttttaaaa atgtgatgtt |
| 59041 | attggaaaaa attaaaacat tcactgctta Raaactaatc atttcattct gtaatctaat |
| 59101 | caagataggK atgtacatat gtatatattt ttctttgttc tttcaattct cagaaaggtg |
| 59161 | tttaaaacct ctatgtcYtt ctttagtcct gtcagaatct gcttcagttt tttgaggctc |
| 59221 | tgttattagg cacatagaca tttataatta ttacattttc ctgataaatt attgaatagt |
| 59281 | tgggtttagg tttataactt actgtttggt ttatatgtat accatttttt tctctgttgc |
| 59341 | ttcttttcct gtttttcttg gggttaatta aatatatttc agtatttgat tttattgctc |
| 59401 | ttactgcctt tttagttgtc tccctttgta tttttgttcg tggttagtct aaaggtcata |
| 59461 | atatgccttc ttaacttact acattctatc ttaagttaat attgtactac ttcacataaa |
| 59521 | aagtaataaa ttttcagcaa tataatttta tttgtactca ctcctttttg atattgtgtt |
| 59581 | cacatattta tattttatta acctcatacc aaatatcata gttttttatt taaatattta |
| 59641 | ttgtctttta aaggtacaga aagaaaaaaa aaaagacagt tgctgggata taatcactta |
| 59701 | tctaccgtgt ctggggctct ttattcctta catatatttg agctcccatt tggtataatt |
| 59761 | taccttcaac ctgaagaact ttctttagag tgtcttgaag tgcagggcta cgtaggtagc |
| 59821 | atatactctc aacttttgtt taccttgaaa tgtctttttt aaccctcatt tcaaaatgat |
| 59881 | attcttactg gatatcaaac tctgaattta cagggttttt taatgttcat cattttaaag |
| 59941 | ttgtgattct atcagttttg ccatatatta tttctgatga gaaattagcc atcatctgta |
| 60001 | tcctgattct ttatatgtag tatatttttg tatgtgttag tggccattct acaagtttct |
| 60061 | cttcattttt tggtgtttag aaatttaatt atgatgttct agatatgatt ttctttgcat |
| 60121 | ttattgtgct gagagttcac taaacttctg ggataggtta catttagtaa atatagagaa |
| 60181 | ttatttcttc aatttttttt accttctttc ctttctcttc ttgatttgag gctcagagaa |
| 60241 | tattcaagtt agatcactta atgttgtctc ctaagtcact gaggccttgc ttgttttatt |
| 60301 | aagatatttt tactccctga ccttcagatt tgacaatttg tcttgatctg ccttcaagtt |
| 60361 | tactgatatt ctgccacctt caaatttttg ttaagtcctt tcagtagctt tttcattcta |
| 60421 | gattgcttat tttttctttc cagaatttca attttttgaa ataattttta cttttctgct |
| 60481 | gagatttcct atctttttat cagttatgac tttcttttct taaaagttca tgatcacatt |
| 60541 | tataatggct tctttaaagt ccttgtttgc taaaatttca acatcttatc atctcagaat |
| 60601 | acattgcatt ttcctggttc ctcacatttc tggttacatt tgattatata ttggacttcg |
| 60661 | tggttgatac attgttaaga cttgggatta tgtctctgaa attgtatatt ctgaaaagga |
| 60721 | ttgatttttg cttcagtgga tagttaaatc actatctgtt ctcttgaatt tgtggagatt |
| 60781 | tagctttaaa cattgttaag gcaggtctat ttgatgtaat cttggtccct agggcaaatc |
| 60841 | ccttggtttt cgtagtccac atatttctaa gatataacct tctggagttt ctggggaaag |
| 60901 | cctgtggttc tcctcaagcc cttctgactt aaaggaactc aaaattcaat ctgtatcttc |
| 60961 | cctgtcatag gcaacagttg taatcccctc tttagcacgt ttagccttct agccattact |
| 61021 | ctttgctaat tttcctagga tctctcccat atatgtgcag tttaggggtc agctaaggat |
| 61081 | tttagaRagt ttatacacag attttggggc tcactcctct gtggcatcct ccttcttgtg |
| 61141 | tttccctttt caatttctgg caaccctagc tgattcaaac tccatcctct gacaagttag |
| 61201 | ttaataagac tgactttcta cttgagctct gtcgacccca tccccgctga actgaagtgg |
| 61261 | gtcctcaatg actaagccta ataaaggagg gctctacccS gtgcagttat tattttcaag |
| 61321 | ggtaccctgc agtttctgcc tgctttgggt tgctctccag tcttttctat atattttttt |
| 61381 | caatttgatc ctgaattttt aatttttatt ctgatagggc tagtccaata aaagctagta |
| 61441 | catcattgct agaattggaa taatttctca aatgctttaa agtactcaga aggctagagg |
| 61501 | atctaaccta aattgtttgt aaggaatgga gaagagtaga aataaaaggt ttcttaaggc |
| 61561 | agtggtaggg aaaataataa agctatttct atattctata aaatcctaat gaatttggtc |
| 61621 | tatttaatac ataagttaca tatagaattg tattggaaag cattaatatc gttattttta |
| 61681 | atacaaaatg ggcaatcgtc agtcctcttc cttcttatta aaaccgtgcc ccttcatctt |
| 61741 | ctattctttt aaaagtcaca aattcagtgt tccaccttta tctttctaag ggctgtttcc |
| 61801 | tgatctgaac ccagattagc aattacgtta aaattagtgt aaagaaaagg cattatacaa |
| 61861 | atttattcta tatagatagt ggttaaaatc cttaatgagt tattatgcta ataaactaca |
| 61921 | cttgctagat gcaaattaaa cataaaaagc attcaaatta gtatgttgat gttgttttta |
| 61981 | atttgtttta tgtgcttaca ctagttagtt taattttgga gacttacttt ttcacctata |
| 62041 | aaaatgagga gtccaagtat gttaatggta agatcacttc caaatactat gttttgaaaa |
| 62101 | tataagttta tcgtatagca atcatacaca agaaatagaa tgttcttcat aaacaatcaa |
| 62161 | tttcaatgta Ytatttgcat gtaagagcat gtcattattg tatgataatt tatagtttag |
| 62221 | agggagtcct tttgcaaata aaaaattgag gcttaaagaa gcagacatat gatttttaga |
| 62281 | cagaaaaaat ataaaattat tcattggaac taggttttta ttttgttcaa ccggatattt |
| 62341 | agtcactctg actaggcttc aggaaaacta ttaagcaggt ggaggagaag aaacctacaa |
| 62401 | aaaagactta gttggagtag atagagagat taaaagaaaa ttgagaaaat gggttgtcat |
| 62461 | gaatgccaac agaaaataac atttcaagaa ggaaagagca gtcaacatta gaaaatgact |
| 62521 | atcaagttaa gggtacacaa cattccctta actgtattct gactgttatc agtagagatg |
| 62581 | taagttataa tttttaacaa ttacaaatgt taattcaatt acataatgat actttaataa |
| 62641 | aataccataa aataatttag ctttccaaga aaattcatca tattcaaact ttatgaggtg |
| 62701 | tatttacccc tataaaccaa aattacaccg ttgaaatggc ataaaaatgt tatgatggag |
| 62761 | ctccagaaaa gcaagcttat taWtgttatt atatcttctg ataatataca tacttttgtg |
| 62821 | aatctaagta tattttaaat aaatgttgcc actaaatatc tctgtacaag acagtaagaa |
| 62881 | ttttaaaatt aacaggatta actcgtgtgc ctttctaagg ggctaccaaa cttaaaagaa |
| 62941 | tgtttcacat agtgcaatta tatatatcat aatgattatg caYgtaccat cacattaaag |
| 63001 | aagagtcaaa gaagaaatgt tggaaagtga gctctgaatt catatgatta ttccacagtg |
| 63061 | attagatctc atgttagaga gctttctcat taaaaccaga agtcagtggt ccagtacata |
| 63121 | catactttta agagaggaaa atgcagtcag agagagagag aaaatgattg atatgatgta |
| 63181 | catatggagt ctatttaaaa actaataata tagtaaagtc tcctcaagtc cttctctgcc |
| 63241 | tcactactct tataattttc tgcaattaat gtgttcttaa aatttatgaa aacacaaatc |
| 63301 | tggccaaaag atatgaaaca gataaatgag agatatcttc tgcctagtac aggagaaata |
| 63361 | tttaagggtg agttaatgta actgagaaat tattttctcc tagtttagaa gggaagagag |
| 63421 | gcccacagag aactttcaat aagaaataaa aagtatatca agtttaggat agaagtacca |
| 63481 | aaaaaattaa aaacaaagat aactctgaat tagatacagt tggatgtttg atgatatagt |
| 63541 | ttcttctttg gctggctttc aagtcagaag cttttaaagt ggagtattta ggggtattag |
| 63601 | aagactttcc aagggcaagc cagtatattc agcacagaaa aatttcaagg tgttgagtga |
| 63661 | cattttgaaa tagaaactaa aaacttcctt tttcatatat taatttgttt ggaaaagatt |
| 63721 | tctataaagt aaaaaaatac agaaattaaa tcttaatgct aaactctacc tcatttaaac |
| 63781 | cataattagt actgacccat ggatatctaa attatttttt taaaaggctc tatccataac |
| 63841 | attgtaagct gcatttgtag taaaacttca tttttataga cactgaatcc atagatcgta |
| 63901 | tactaatYga atcatgactc aattcagaaa aaaagatttt atacctaaaa ctttatagtc |
| 63961 | actggttatt taaaaaggat tgtaatttta atttgtatgc atatttttga tgcaaaaata |
| 64021 | ctttggtgat caagaaaaga ctttcaaaca cacatttcta ttgtacttga attacaattg |
| 64081 | tgtgaggKtg gggcggaata taaatatagt tcaagcataa aataaaatga tataaatatt |
| 64141 | ctacctatta aagcatgagg tcaaatcact atgatattta gattctaaca gatatatttt |
| 64201 | taaaagtgac ttgacaatat tattttaagt tatcaatatt tacaacacaa tatacatatg |
| 64261 | tcagtgccga cgcccactca ggaaaatgaa aaccattcct aaaattttga aaagaggaga |
| 64321 | atttaatatt gtgggttgat cacaaaggcg ctagaaggaa tcaaagagca aaggatgaac |
| 64381 | agagtaggac attggaaaag caaaaagaaa aagtaaaaca cagatatcaa gtgttactga |
| 64441 | tgtgtcagtt gattgtatct gccagacgta tgccccttga tcagacctgg aattgccaaa |
| 64501 | aagttgctgc ccgtgcccta gctgctggga tcaagaatcg cctactgcca ctgctgctgc |
| 64561 | agccactgaa ataaccattt aaagcagacg gctcttctct ttctttttct gtctgatatc |
| 64621 | ttcagtggtt tccctcaggt ggaaattatg gagaagccag atggcaaagg agcctgggaa |
| 64681 | atctggtttg cagattccca tccctataag gctcacagta tatagcagag tagaaaatgg |
| 64741 | tatgtatagg tttaagagaa aacaggtaat gatcagtact gtctgcaaat tgcgcccttt |
| 64801 | acaattttct aaatgattgc aaaattaata tgtcaattat aaatgtagaa agatacatac |
| 64861 | ttaaaaaatt cctttggggt ttatatgaag aaaaaagctt gatggttatt acaatggaag |
| 64921 | atttatttta tataaagaac Wtgactggtc gttttggatt taaaaataat ctgttccttc |
| 64981 | agcctaattt tacctgagcg tttccaccac ctggaaatta aatctcttca tgctaactga |
| 65041 | ttatcaaatR ctaaattata tgacaagaat tttactcccc aaggaaaata gatccaaaaa |
| 65101 | tggaagaaag ccagaaccca ggaaaatgga aaaaaaggag gaataagtgg actaaattat |
| 65161 | aaatctgtaa aattaggata aagtactata cttagcatgt aagtattaat agtgatgttt |
| 65221 | ccatgttaaa atatagcacc attgagagtc ctatctctca atttctaaac ttctagtata |
| 65281 | acactaggct tttcttgtca ctgtgcccac ccacgtggtt tcatgagagt aaagaatgtt |
| 65341 | tttgaaagta gacatataat attggagtat aataagatac tatttagtaa ttataatgcc |
| 65401 | aataatgtca ttttctgaga aagaaaatgc tcccaagact aaacagagta aatcagctgc |
| 65461 | ttgggactac acagctttcc taatttttat ggttgacaca aagtttggca attctttagt |
| 65521 | tttactaggt aaatgaattc ctatgatggg tattttagtt catcaatgca ttaacaatag |
| 65581 | aaagcatata tttaccggga ggctttataa atattttatt ttattatttt atttatttat |
| 65641 | ttagagatgg agtcccactc tgttgcccag gctggagttc agtggcgcaa tctcggcttg |
| 65701 | ctgcaacctc cgcctcctgg gttcaagcga ctctcctgcc tcagcctccc aagtagctgt |
| 65761 | gattacacgc acccaccacc atacccggct aatttttgta tttttagtag agacagagtt |
| 65821 | tcgccatgtt ggccaggatg gtcctgaact cctgacctca ggtgatctgc ccgcctcagc |
| 65881 | atcctaaact gctagaatta ctggcatgag ccactgcgcc ctgcctataa atatttcaaa |
| 65941 | acatatgcca cttaacatgt gttaatcaca tgccatggtc aaagtgctat ggagggatgc |
| 66001 | aagtgtgaat aagacataga cattgatcat gaaaacattt tgttgatggt ctctatgtgc |
| 66061 | tatgcactgt gctaggcact ggagatacaa agataaataa aacatggttc ttgcccttga |
| 66121 | agagatcttg tcttaatgag aaacacagag gttaaactag acacttataa tacaatgcac |
| 66181 | taggtgcaat gatagagaag cactaagaaa gcaacaagga aggtagagta aaggtaaatt |
| 66241 | tcctgaagga ggagaaatat agcctgatga caaaataaca ggtagtaatt atctaggtaa |
| 66301 | agagggattg gcagaaagtg aaggacgtct actgacagac acaagagtag aagtaaaggt |
| 66361 | agaaaggtta gaaacagcat ggaaagcagg ccaaaattta tgagaatttt gcagctctcc |
| 66421 | aagcaaaaag tgatgagaat ctatcctacc atactaatgt tgtgaagaga atgaaagaca |
| 66481 | ggaaatcaga gataaaaagg aggtaggata aacagaattt attggctaaa gtgaggagaa |
| 66541 | gggagaagtc tggaataatt catcagttca tagtacaaat acataaaagg gtggttgtgt |
| 66601 | aactaattgg gacagagatt gctgaaaaag atttagagga ggaggacagt ttagaatagg |
| 66661 | gtgagttgga tatgtagcat tctgaagctc actgtgaaag tgccatagga ggtatacatt |
| 66721 | tgggagtcag caacctgtgg gtggtagttg aaatcatgaa agtacatcag attgccaaaa |
| 66781 | agaacacatg gagtaagagg atcactgggc taatcttagg atgaaaccca gagaaagctg |
| 66841 | tcacttaaga attgacaaaa taagaagaat ttatgaaaaa aaataagaaa gtttcaaaaa |
| 66901 | ataagctatg actagcaatg tcagaactat ttgagatgtt gatgataaga actgccaaat |
| 66961 | gttcactaga ttggccattt gggagttgtt ggttacccaa gccagaactc ttttggtagc |
| 67021 | atagtgggag aaaaagccag actgcaaaag atgatggaaa aacttggatg tgaaaaggtg |
| 67081 | aaaatgacat gcatatgtat tattttttca gaaattttag atggagaggg gaagaaagag |
| 67141 | ataataattg gaagggtcat tggttttggt agagtttttg tttgttttta agttaaaaca |
| 67201 | gagacttgag taaaattata agctaaggaa gaaaaagaaa gaaggaaaat gtaatataag |
| 67261 | aaagtcagtt aatatgtgaa ggataaatat cctggaacag attcaactcc atcaaaaatg |
| 67321 | attaaactga aaattgattt taatgaggta aagatgttac tttgatactg aatataatat |
| 67381 | aatgagaatg tgtatgttaa taatagatag gcaagagaat ggaaaattga caaagatact |
| 67441 | ttcttattcc ttcaaattta ttgataaaaa ttggagatta cgtatgttga cagtgatggg |
| 67501 | gacatgtatt atgtaacagt ctacaggaga gtggtgaaga ttgaaataga cattgaggaa |
| 67561 | agggaaagtg actgctaaat cagaacagat aaaagaatag atcagtggca ctaaagactg |
| 67621 | agtggaggtt tatatgaaac tgacactttt ttattgctgc tagtttacac aacagtgtga |
| 67681 | ctttcttcag ctgagattga gtgcccaagt ctaggagcag agatgaaaat gttcatatca |
| 67741 | aggttgagac atggtggata aacttggtaa gggaattaaa tgagatgaaa gaagagctaa |
| 67801 | aatgattaag gaagcatagc catgatcaga gacataaact tggataaaat tttggaaatg |
| 67861 | ttaattagaa gatcaaatga agttgaaaaa caggtgaagt atgagtgaag aaatgaaaga |
| 67921 | gtaggttaga aagacataac cagagactga aatacgaaag ttctagaatt tgaccttgag |
| 67981 | gttcatggca gagtgttaga gaaaacaaaa gaaatgagtt taaaatagct caaaatggag |
| 68041 | tgagggaggg agggatgaat agatgaagca taggggattt tcagggcaat aaaactattt |
| 68101 | tggatgatac tctaatgatg gatacactgc tgacattata cacttgtcaa aacccataga |
| 68161 | aagaatgaac tctaatgtaa actgtacacc ctagttaata atcatgtatt gatattggtt |
| 68221 | cattggccag gcaaggtggc tcatgcctgt aatcccaaca ctttgggagg ccaagatggg |
| 68281 | aggatccctt gaggccagga gtttgacacc aatctgagca acatagagag acctccacct |
| 68341 | ctaaaaaaat aaacattaaa aaaaattagc tagacatggt ggtgcatggc tctagtccga |
| 68401 | gctgttgagg aggctgaggt gggaggatcc ctttagctca agagtttgag gctgcagtga |
| 68461 | gctgtgatag caccaccgca ctctagcctg gatgacagag tgagatccgt ctccaataat |
| 68521 | gattgtaagg ttcattaact gtaacaaatg tacctcacca atgcaacata gtaataatag |
| 68581 | ggtaaattat tcacactttc tgctcaattt ttctgaaaac ctaaaactgc tttagaaaat |
| 68641 | aatatctatc tgaatcatga cccaaaatga ttgttgtaag gaatatctac attgattctg |
| 68701 | aaatatatta ggaggttggt cagtttttgt gtgaagagaa acatttagcc aggtctcaaa |
| 68761 | gttttcagag aaggagaaga agatgctatt gtgaggtttg tctggtggag gtgatgagac |
| 68821 | ctcagaaagc acatgtaatt gacaacataa aacaaaacaa aacaagaatg gagatagcat |
| 68881 | gaatagagtc tcattaataa ataacaaaaa acaggttcct tgagtatcta agaaaggcaa |
| 68941 | aattccatgg ccYctaataa atagtgaaaa tgatggaaga gaattcccac gccatgtcag |
| 69001 | ctcaaaacaa gtatattttt tgcccaagcc atgctcaagc tcttgctctt tttctatgcc |
| 69061 | tcctgtgctc atccccactc ctattgtcta ggatcccgca cactgggtta ttgatgtagc |
| 69121 | tagtcatgac acttggctga tggagttcat ttcaataccc tagtagctaa tgtcaataga |
| 69181 | actttcattg attttccagt gtcctttaat tcgttaggta ttgacctttt atttaaatcc |
| 69241 | cacatgacca ttccaaacat tttccacact tttcaagcct caatttctag tcccttcatt |
| 69301 | ttttacaccc tcgtcatgtt cagttgttaa actctatggt ttactgcaaa agctgtgaga |
| 69361 | atctagcgta agtcccttgt gttcttgata ttcaacctca aaatccttcc atgattctct |
| 69421 | catctttagt gacatcaaag gcattagtta tggatactgt tgacacattt aatacccatg |
| 69481 | tgagcctaaa agagttttga atcctctctg agtcacaatg tccacatcag tggaatgaag |
| 69541 | ctattgtgaa gatgaaccaa gaaaatgaat tcaagaatgt gttgcaggaa aaacaaatag |
| 69601 | atcttgttgt atttgttgac tcggatagat tggtaaaact tcctcaggtt ctgtgtttta |
| 69661 | aaaatgtaac agatccaaca catatttgga aggtagtctg ataggatttg ctgtggggaa |
| 69721 | gtgagagaaa gaccaaagcc gagaataatg gtttccagct tcatccatgt ccctaaggag |
| 69781 | atatacctaa tgtaaatgtc gagttaatgg ttgaagcaca ccaacatggc acatgtatac |
| 69841 | atatgtaaca aacctgcaca ttgtgcacat gtaccctaga acttaaagta taataaaaat |
| 69901 | atataaaaaa aatttaaaaa agaccaaagc caacaatctc ttctaaactc atcccttttt |
| 69961 | aattttgtct tttcagccac agcagcagaa aacaaaaaca aaaacaaaaa aaaacagcct |
| 70021 | ttatttccat tttccccagc tccactcact atcttgttcc tagccacaat tatctcttct |
| 70081 | ctggattgta gcYgtagagg tccaattact ctccctttcc actcttgact gccttcaata |
| 70141 | tattttaaca cagcagccag agtgatccag tcattttcct gctagaagac ttttgatggg |
| 70201 | tttccatccc actcagagta aaaactgtcc ttaaaattgt ctacataatc tgcccaatca |
| 70261 | attccctttg tgatctcttt atctctccct ctgtcaccat gctccaagct tatggtaccc |
| 70321 | cttgtacttc cttatactca ttgaacttgg ccttttcaat ttctgtctct cctatctgcc |
| 70381 | taaaatgata tttctctgtc cacctcattt ttttttcagc atttgcttaa agcttattag |
| 70441 | agaggactgc cattgccacc ctcttcaaaa gaacaacttc catcctttgt actacctatc |
| 70501 | tctcttattc tgttttattg ttttttataa cacttacaat tattttatta tgtactgact |
| 70561 | tgttcatttg tcttcctctg gtaaaatgta agctgaagtt ggtctgagat ttcatatatt |
| 70621 | tggtttacta ctgaagcctc aatatctaga aaatatttgt acataaaaga atgaatggtg |
| 70681 | gccacatggc atacattagg taataacact cccctaatta ataaccattc aatgggatct |
| 70741 | cattgctctt taataaaatc aattaaccta ctatagccta ctaaatttgg actttatata |
| 70801 | aaatgttttt atctttgttg aaagactaca ctttgagctt ttgttcctat tattttaaat |
| 70861 | attttgtcca ttaattaaac agcttgttat attcagctac tctcacttta cttcaagtta |
| 70921 | attgagaatg gtaccattta ttggcacaga agaaatgaat gaaggaaata tttggccagg |
| 70981 | agttgcagaa tgcggaatca aaactactat tttagaaatc agtaataata cccagatgct |
| 71041 | attctaaagc tttacaggta ttgactcctt acatgctctt actaatttga tgaaatagat |
| 71101 | agttaatatg cccattatgt agatttaaga catcaaggct tagagaattt aaataccttg |
| 71161 | ctcaaggtca catagccaat aactggaaga accaggatat tagcttaaat tgtttgattc |
| 71221 | cattctctta acaactatgg atatacctct ttggagctat ccaaatggaa aatgaaataa |
| 71281 | gtaatctgat atatgagtct ggagttcKgg taaggatatg gaatggacac atgcatttga |
| 71341 | ggccagtctg catatagatg aagtttaaat tgcccctcta cctttcttta tccctacttc |
| 71401 | tcttttattt tcagtctctt tcaaattctt tgctccttcc ccatttctta aaaaaggtat |
| 71461 | tctctatctt gaaaataacc tgctcatgta ttctaagcta aattaattag gtgtctttgg |
| 71521 | ttgcaaatga caatccaatt gaagtgaaaa gataatttaa ttctcacaaa actggcctat |
| 71581 | tcagggatga gatccaggct cctttcatca ggctttggta tcgcttgctc catctctcag |
| 71641 | ctgtgatttt tttgtgtgtg tgttgcccca gttctcaggc aggctctcct ctcttggcag |
| 71701 | taaagtgact tttagtagct cttaggctta gatttcatcc tcttatctat cccaacataa |
| 71761 | acatagattc tcttttacct gcagttccaa tacagagcct cagctttaac attcactggc |
| 71821 | tctgactatg aagtattgtg ttcaagagga ggcagtgttc tcaatgccag gcctacacca |
| 71881 | cttgcccagg tggagcactg agagtagggg tagctacacg caaaacacct agagcaagcc |
| 71941 | tcggggagta gtgaatccca aggaaaagta ggatgctgtt atcagaggaa ggggaagtag |
| 72001 | ataccaaaca ggaaaaacaa aagatatcct tcaggggctt aaaaacaata gaataagtta |
| 72061 | caggaaacta tcaatagtta catgtacaca aaaatttaaa tcgacagtaa tacaaaatca |
| 72121 | taatgaacta aattaaacta caaacaaaat gctagaaaat atttgtaaaa gtgtaatggt |
| 72181 | gacttgtttg tgtcaccgct tcactgctac atgaagtaaa aatcaacaag aaaatactaa |
| 72241 | gaccccactg gatagatata aaagatatga acttagagtt tcaaaaggag aaactagtag |
| 72301 | gcagtataaa tccgaaagca aatatattgc aatggagaag ggcagttaat cattttttag |
| 72361 | aaagttaagt ttatgaaata tgattgatat atattttatt ttacaaaata atgtaaatgt |
| 72421 | gttatatatg cttggaatta ttttatatgt tgtatttcat aataaagtag aacatcacca |
| 72481 | gaatttgatg acatacttgt catcaataat agatttgaca acactgcaat taacacaaaa |
| 72541 | cccacaagga atttaaaagc atagcttgaa actgaccaaa taacaactat actagcactg |
| 72601 | atatagagct tacttattcc tgacacagtc tcacatacta tgtatttatt acctctcagt |
| 72661 | actcacaatg ataagtacta ctattatctc aatgttgtag atgatgaaac agagagaggt |
| 72721 | gaagtaactt gcccatagtc acatagccaa gttgtagaga attaattatt caaattcagg |
| 72781 | aagtttggct ccagcatcca agtgcttaac tcctttacca gtttccagag aaaaacagtt |
| 72841 | ttgtctgcaa agcatttaaa taaacccagt tacatgtcag tgagaaaaac aatactcaaa |
| 72901 | cagttcagag ctgttttttt ttaaacctgc atatttgaag aaggttcaga taggagttca |
| 72961 | gacagagtat gagtagaaga caaaacataa gattacatag agaaagatWt ggggtgagat |
| 73021 | gtaaaagatt tgcatttcta aacttctcga aattcacact tctttctcaa acacctgtgt |
| 73081 | cttctttcca taaaaaatta aaacttttag aaaggactgc aatatactgt acagtctata |
| 73141 | tctcacttcc ctcttgcatt taatcacact ccatttagat gacattcagc tttaatctgc |
| 73201 | ctagactgcc tgtactcact tcatttctca accctagcta aacttgcact ttgtcccata |
| 73261 | aggctttcaa actactccca tctcttagta atctcgtcct ctgagaacac ctattccact |
| 73321 | tcttttcaaa ttacctttgt gtaatgttac acatctatct atatatatct acatctatat |
| 73381 | ctataaatgg gcatatatac aatctcagga agagagaacg agagagatgg agggagagag |
| 73441 | agaccttctt tttgcatagt ccctgttttg gaaatcagaa gactaaggtt atgtttactt |
| 73501 | tgtatgccaa gcacacagca gaatgcccta aattcatcca tttatctgca catgtttttg |
| 73561 | aatgcatgtt gtattctggg cactgtgcct ggcatcatag atacaaagga gtggaagata |
| 73621 | cttttcctcg gccttcaaag atctctaagt gcacacgaaa aacaaatgtg tagataaata |
| 73681 | attaaaacac agtgtgaaaa atgccatagg gtaatacata cagggtgcca tggcaaccca |
| 73741 | ggggccgaat ggcagcttct gcctggggag ttgaaaagat ttcacagaag aggagaaatt |
| 73801 | taagctaggt cttcagatga gtaggagtat aagaggcaac aatagggata attataaaag |
| 73861 | catgtttgcc ttgataaata gtttgcactt tatcatttag ccagtacaaa ttataaaaga |
| 73921 | gttttacaaa agagaaaaat gtgatttaac ttacgtttaa gaaagatagc tgaggtaggc |
| 73981 | ctgagaatgc attggagtga gRaagagaaa aaaacaaatg gctttttcaa caatctaggg |
| 74041 | aggaataatg gaatgtctac attaaaacaa cagtaacagg agactaacaa aaagaatgag |
| 74101 | tctgaggaaa gttaatattc tttattttat tttattatta ttatacttta agttttaggg |
| 74161 | tacatgtgca caatgtgcaa gtttgttaca tatgtataca tgtgccatgt tggtgtgctg |
| 74221 | cacccattaa cttgtcattt agcattaggt atatctccta atgctatccc tccccccatc |
| 74281 | ccccacccca caaYagtccc cggagtgtga tgttcccctt cccgtgtcca tgtgttctca |
| 74341 | ttgttcaatt cccacctatg agtgagaata tgcggtgttt ggttttttgt ccttgtgata |
| 74401 | gtttgctgag aatgatggtt tccactttca ttcatgtccc tacaaaggac atgaattcat |
| 74461 | cattttttat ggctgcatag tattccatgg tgtatatgtg ccacattttc ttaatccagt |
| 74521 | ctatcgttgt tggacatttg ggttggttcc aagtctttgc tattgtgaag agtgcagcta |
| 74581 | taaacatatg tgtgcatgtg tctttatagc agcatgattt acaatccttt gggtatatac |
| 74641 | ccagtaatgt gatggctggg tcaaatggta tttctagttc tagatccctg aggaatcgcc |
| 74701 | acactgactt ccacattcta tggcataata atagaaattg ataacttgga ggagaaaaga |
| 74761 | aggaagggtt gagacttggt gagaaattta gcagaggaaa atgagcagat ggacactcca |
| 74821 | caacaaaaat aggggatgca gaaggaaaag cagattggaa aagggcaaca gggacacaSa |
| 74881 | atttggctat aggcattttg attttgtggt acctgaggaa cattctcatg gaattgtcta |
| 74941 | ggaacaatta gtcagtgagc atctggaacc caagtgaata atgttgagaa tttctaaaag |
| 75001 | tttaggaaac ataggtattt ccatcacttc cattataaaa acttcattgg gaatgttgga |
| 75061 | ttaataggaa gatcatgcac agaatgatta tttaaggtcc taaattgttc cacctgagca |
| 75121 | tgggtaagga gaacaggtgt taattatttt tgctgcgagg ggaaataatt taaaaactag |
| 75181 | agggaggaga gggttaactt tgggaagaga gttcagagaa cctgagaatg tgatgagttg |
| 75241 | tccccttttc cacccacact ggagaggggc atgccagggg cagttgggaa ggatcaggca |
| 75301 | ttcttcccat aaagtcgagc ctatctctct atgccagcag aacatgctgc agggaggcac |
| 75361 | aattcccacc attctgaccc accatttcct aggtagtcaa acttagggtg gcaagaacac |
| 75421 | ctaagattcc cacagtctag agaaggagga agagctggca aggacaaaga ccagaggaaa |
| 75481 | tggcctcctt gtgcagggag agcccaccac ccactgggat caggagaagg tcctgttatg |
| 75541 | aggagcaacg cttgtgaatg aagaagggtt ctcagctggg tgcctgaaag ccatcaaaaa |
| 75601 | tgttccatga tcacacaaaa gagtcttaca tattttcatt actgcaaacc agccttgaaa |
| 75661 | atatatgtat gggccaagaa aactttgccc accgaagaag aacctccctg ctacaacaca |
| 75721 | cctgccagtg actgcccagt gataaatgct tgcaggcctt gtgaaatgag gagatagctt |
| 75781 | tgcaaagggg accagagtaa aatacgtaaa aagaagggaa gcccttatag agactttaaa |
| 75841 | ttggacagtg gctaagactt tcattcttac atgagattaa acaagtaata atagacagat |
| 75901 | tgagtttccc agagtatgtc agctgtacac aggcaggaag ttaaaaaaaa tacatgtggt |
| 75961 | cataagaata catgtttgca cgtctgaatg gtaagtgttc aaaatccact ggatatgcat |
| 76021 | ataggatgaa actaaaatat agatatggga gatcatacaa cctgtttgta tataagtaca |
| 76081 | agaggaaaac atggccaaaa atatttgttg ttgataagga aggattttaa cactaatgaa |
| 76141 | ggaaaaggat aacgagagat ctgtgaaagg aaataaatta tgtagatgca aatgacttag |
| 76201 | tgaatggctg taggagcaat tttctaaatt tgcaggtgat atttaattgg cagacattgc |
| 76261 | aaacagcaag gagcaattca atcagataaa gaacttgaat cattaagtac ttggcctaat |
| 76321 | agatggcaag cgcagataag tggtgaataa taagtcttga atcaaaacta aagaacagag |
| 76381 | agtgaacatt tgccaaaggg aatgatacca tccaggaatg gctcttagat ttaaggagaa |
| 76441 | cttatagaca aggacaaaca aaacttcagt aaaatattac aaataaaaag atttggtggg |
| 76501 | ttacagctta tccagctgaa gctgtgatga gaaaatctga caacttaagg atagataaat |
| 76561 | cctgaaatcc acggttggct attgaaattc catttgttac ctagaatttc atctatccct |
| 76621 | gatcatggtt tggagataaa attaatgagt aagataacat ccaagcatat aagaggttac |
| 76681 | agatttgggt gaggatacta taaataactt ttaaagacat ttcggatatg aaattctcta |
| 76741 | tgttctatgg gcataattgt gaggtattgt gtttagattg cttggtcatt tttatttaac |
| 76801 | atgccagttc tagtctacgt tagtctggaa attataaaca tagagaaata tgctacaagt |
| 76861 | aaaagagaat aataaaaatt tattatagcc tagaaaggta gttgatgcaa tatataatat |
| 76921 | atttcctctt tctctKtttt tgcaatggtg aatactttac taaatgaaaa ggttaatttt |
| 76981 | aatattaaag agaaatggaa acatccttta gtacaacatt aaaaccacaa atggaacctt |
| 77041 | taaatattca ctcactctag caatcaatgt tccaacctca tttaaaggat tttttttaac |
| 77101 | ttgacataca aatattcaga atgaatattt ggaagcagag ccacgagcca taccttgaaa |
| 77161 | gataactgcg gacttactct tgcctgccat ttggRtactt cagatttcag ttgttctaag |
| 77221 | tctcaatgat cctgagtttt ctttttgtaa tttggtaatt ttgtaatggc aggctttaaa |
| 77281 | ataagtaaag gttttcttaa ctaagaacaa aaaaaaatcc ggagacctaa caaggctcag |
| 77341 | ctgtgtccag gtaggagatg actgcagttt ggcttcagag gctccctcta gcagtgtgag |
| 77401 | gacatctgag tgtctctcct actcatttta tatttcattt gatactgcac agaatcacta |
| 77461 | ggagaacagc cagaattgat ttacttctca tagagacaaa ctgtttctgg gagctagtta |
| 77521 | tcttcttatt aacaaagagc ttacattccc ctcccctcca gagctcctaa aacatctata |
| 77581 | taatagctac attaagtgaa ttctacaatg attgaaaatt ttactataat gaagataaaa |
| 77641 | attcactcaa ttctagttct tttatccttg atttgcattt gaYtgaaatg tgtatttgca |
| 77701 | gtttgggcat ttcagtcagc gattgctcct gtattctttg cccataagca gccttcagat |
| 77761 | ggcctcttag aaaattgaag aagtctctac atgactggaa aactctttct gatggtctgt |
| 77821 | ttcttcaaag atatagcatt acctcttggg aattatagaa acaatatata ttgttaagcc |
| 77881 | tcttctaatg ctactgtaat ttataactgg taactttttc ccccacttag cattcaataa |
| 77941 | gcatttattg gggcctaatg tgtaggtgga attttggaga atacataaag tatagaacac |
| 78001 | agagttcttg ttcttcaaga gctaagcagc tctatagaga agagagagac agaacgacag |
| 78061 | agacagagac agagagagag agagaagaga tcaacacata tccaccactt gctttactat |
| 78121 | ctatacttca acttttggtg gactcatccc atcttatgga ttcaattatc atctataagt |
| 78181 | agaccacttc cccaattgtt tctttaaccc agacctctca cctgaactac aggcttttat |
| 78241 | tcctcacaat gccttcagca ttttccattt ctaatgggta ccYcaaacat taagtgttca |
| 78301 | aaactgaatt tctgatttca cccctttccc Ycatccttgt ctgttgtagt gtatggcaac |
| 78361 | tccaattttc aagttactca gtgcaaaacc tttgagttgt ccttgaattc tcttctctct |
| 78421 | gtcacgcctt atatacaatg catcagcaaa tttgtctggc tgtaaatctg aaatatacca |
| 78481 | taaatataaa tacgatcact tctcaccact cgaaagccac tatttttctc tctttaggac |
| 78541 | tatttcaatg gccctttaac tgctcaccaa gctactgctc tttttcctat ttctgttaaa |
| 78601 | aaatctattt ttaacagagc agccaaagtg attgttttaa aatgtcagat cgtattacac |
| 78661 | tctccctata accctccaat agttttcctt caactctaat ttacataatt ccctataatg |
| 78721 | ccttatatgt tcaattatat acatatacac ttaccaccat ctctgtgaaa tctctttctc |
| 78781 | ctacttccct agtatgagta ttgcttaggt ataaatcaga actcagaaaa ataagagtaa |
| 78841 | gaattaaaaa ggacatatgt gctgaaaaaa atagagcaat gaataaatac tggggaagta |
| 78901 | attagaagga agagattata cagaaaccat aaggcaaaaa gttaatcaaa tttatcaagc |
| 78961 | atattatagg ggacatggcc aactccacaa tttgttcacc tataatctca ggcaagctat |
| 79021 | ttaccttgtc tgagtcttag atttggcctc agcgaaagga aagtggtgat acatcatgag |
| 79081 | cagggttgca gcacagctgg cataaatgga ggtttatccc acacatgtgg gctgaaaata |
| 79141 | ttagaatcca agctaccaag gtcagagtgc ctggctaaga gcttcatatg gaaaaaaaac |
| 79201 | ctttaagtca atgacaaaat gggagagact tttttttttc aggcagtata tagccaagaa |
| 79261 | tctattcctt gaatgctgga attgaaggtt atataaaagc acttattact accaaattgc |
| 79321 | aaagaacaag cagagcaagt aagttgagag agagagagag aSatctttct tggagaacat |
| 79381 | ttttggaaaa tgactgtggg caaagcaatg tgaaaatatt tagggagaca gaagactacg |
| 79441 | aagataaaat acctatatca agaagtttat attgaaattg agaatataaa acagatatta |
| 79501 | tgggataagt cccataggag agatacagaa aaacagagaa atgaaggata actgtaaact |
| 79561 | ctagggaggc aatgtcacaa actcaggtga aaatacatta gaaaaggaaa actaggtatt |
| 79621 | tttcagtata tataatcagg ctggaagata aaagctggtt cagagactag tgaaaacaaa |
| 79681 | tcaggctgga agataaaagc tggttcagag actagtgaaa actaaattgt tcctataagg |
| 79741 | actttaaaag gagatcagca tgtgagtaga cttggaggcc tgctaaagag ggagctagga |
| 79801 | gaaggaatag cacgtggttt ctgagaaaca gtgtagcaat aaggcttatc acacataaac |
| 79861 | taaactaatc atgatgtttt tgtcactatt gtgctgtcag agaaaaaaat ctattgaaat |
| 79921 | aatgaaacat actaattttg agaaacatca gtgtggcaaa acccaagaaa cacttttcaa |
| 79981 | atgaatatac tcaaattgca agatcaaaca tctattaatt ataataggta agaaaaagga |
| 80041 | aacattcaaa aaagtccttc aaagcgaaaa tatgtaaatt agagtacctt cataatcaat |
| 80101 | aaacacttat tgagcatcta ttcatgtgct atcaatgtgt taaaaataca aaagtgacaa |
| 80161 | aaataattat tgctcaaaat tctgtgcagt atttagaaag attgggtagg gataaacatg |
| 80221 | tatttctcta agattacatt acattatgat aaggtttatg gaaaaaatgt acatacctat |
| 80281 | gtatatgtaa aaatagaaaa gagtctaaaa gaactttttc aatctcaaaa cagtgtttac |
| 80341 | atatgaggaa atgtagYgat gactgttcat tgtttataaa tatctgttct ctcctttctt |
| 80401 | agtaatacag ctcctgaatc ataatagcca gatacagact tcccacaata tgaatttccc |
| 80461 | agtttctctc cttagctagt gtggtcatgt gactaaattg tagccaatga tatttaagca |
| 80521 | gaaatatcat gtgatgcctc tgagaaatgt tcttaaagga aggattttta ttcttattct |
| 80581 | tatcttcctc ctttcttctg gctggaatgt taataggatg acttgtgttc aagcagccct |
| 80641 | ctaaagacat gaRttggaag ctatgtactg aggagcacag agcaacaaga gaaaataagt |
| 80701 | aaaataagtt tggttcttag ctctggaagc gtgataaacc tgggatagtc agccagggaa |
| 80761 | cttctttcat gttgattttt aagccagtga tattttgggc atttctttta cctgcaggaa |
| 80821 | gaaaataggc tgattttata taatttgacc ctttaaaaac tagaatatat tcatacatag |
| 80881 | tttttacagt tcagtgttta aacttattta tttttcatct tagttttgct tccctatgtt |
| 80941 | ttgtaaatgg taaacttatt agccattctg tggtatataa aagtttcact tttatgtttt |
| 81001 | gtttttatat tggcccaact tccatgttca gctaaataca ctaacataat ttaaattact |
| 81061 | gcatttcatt gggaatccaa gttagaaaat tccttttccc acatttttaa tacctaaaac |
| 81121 | ttgggaaatg aataacatta taagtacttg aaaataatat tcactgattt atcatgtgta |
| 81181 | agccctgtcg aaatatttat tttctactga gcttggaaga tattttaaaa tgattccatt |
| 81241 | aaagaaattt ttaaaatttt tgaaatatct atttacttaa gatgttgaag aaaacataag |
| 81301 | catctacctt taccttccat tttttagatc tcatatgata tgacaaataa atagaaaatt |
| 81361 | agaggaaatt ttacaacagt actctaaatt aagtaagggt accctccatg aaccaggaat |
| 81421 | tggcagattt caaaaaagca cactcctaag aaatttctgt ttttcacgtg accctgacaa |
| 81481 | aatccattaa ttgtcctaat ttggaagggt acaaactgca gaaaagagca gggtattagc |
| 81541 | tcagcaggaa gcataatatt tcattgccaa gttgctgttg tgactcagta gcagcacttc |
| 81601 | tgttatcttc tgcatggctc ctcactcttc agcgagcaaa ttaggactca ttcacatggt |
| 81661 | gatccagggt tcctggcaca gcaagataga tggcaagcca ttgtgccagc cctccaatgc |
| 81721 | tctgtttgta tcacattgct tttctcccat tgatcaaagc aagttcctgg ctaatcccaa |
| 81781 | agttaaagga tagggaaata gactgcacca attgttggaa gagctgaaca gtacattgca |
| 81841 | aaggggcagg aataccagaa taggaagaac tttgtggcta tcttatgtaa tctgccgtgc |
| 81901 | tgatcagtta gactaataaa ccaagagaag aaatgaagga taataaattc aaatacctaa |
| 81961 | tagagatcag gctgttaagg tataaaagta aagttgggta tagtacctat gcacacaatg |
| 82021 | tgcttgaatt gtgagagcta aagtgaattg aagatctcat gaactttcca agggggcact |
| 82081 | gtctctcaaa ttcaacaagt tgcttgaaac agactccagc aattttcaca tgaaattgta |
| 82141 | agtttgaatt tctatgtaga ctctactaat tttgaattct ggctgaagta ttttaaaact |
| 82201 | gtggttgaac ataaccacat ctgtaagtta cttatgaatc atggaccatc aacatgtgag |
| 82261 | ttttgataga gaagactgaa aggataacat tttacaactc tattaaatat aatataaata |
| 82321 | gtacaattaa tatatatttt atataaattt gtgtcctata gacactgaag acccataggt |
| 82381 | taattacaaa aattgataat acatcatgtg cctccaactc caaaatttat atacaacata |
| 82441 | agtaatacct aacaaattac cagattaaaa cacaataaaa ttaagatgaa taacatttta |
| 82501 | aacaaagaca aaatctcaaa ccacttggaa gtaaaaatag caaatgagca aaaactctca |
| 82561 | tacactgttc ctagagcaaa gaggaaataa aaactaaaag tatagactat ttagaaaata |
| 82621 | atgataacaa aagcactaca tgtaaattgt ttttaaaact ttgcttacag aaacgtgttc |
| 82681 | atctttaaac tctacttaat tactaaatta aaataatgaa tcaagccttc aactcaataa |
| 82741 | acaaaatatg aagccattaa agcttgagtt aaaatgcatt aaaatctgta ttttagaatt |
| 82801 | cataaatcaa tagggttaat tgaagggaaa aaacagttct ttgaaaagat ataaaacaaa |
| 82861 | aacaagcctt gggcatgtct ggttttggaa aatgaaatgc ataaataaaa gtatcaaaaa |
| 82921 | catgtgtaca ccacaattaa gaaaaatttt cacaagcatg atggaacagt aagtacaact |
| 82981 | ttatcgtaat tatttgaaag aaacgtatgg catgaattat tatcaaggaa aacatcccat |
| 83041 | cattaaaatt gacccaagaa gaaatagaag acaattaaat atatgtccaa tatgacaaaa |
| 83101 | aatgttattt gtttacaaac aaattaataa tgttatctcc tgcctgtttt cttaatttca |
| 83161 | ggaaagagta ggccaaatga ttaagacatt tcataaaagt taYccaatta attctgaaga |
| 83221 | gctagagtaa ttcccaaaag aatggctgaa aatgaaatca catacaaaaa aagaaaaaac |
| 83281 | attcaatatc actttagtag tggtaataaa aatacttatt aagtgataat tgaattcttt |
| 83341 | tacaaaattc ttttggatat gaatatccaa aaatcttaaa tgtcctttca tatttaatcc |
| 83401 | gtcagcatat tagaacatag taaagtatta ccaaacaggg tttattacag aatgagcttc |
| 83461 | attcaaccaa aagataacag gagaatcttt ggcaaaatat ctaatagtaa gcaaagttgg |
| 83521 | agacatggat tagataataa tatgaaatca tcatgtgttc taaaaatgaa agaatggtat |
| 83581 | taaaaaatca aaggaaaagg ggaaagtagg ggaaaatagg atatcgtaat gcactttagg |
| 83641 | gaataagtgg aagtaacaga ttttatttaa aattttcaaa acgttaactg gaaaaaaagg |
| 83701 | agtaaaagca taataaaatt tttaaatatt gaggttaaaa actataataa aagtgcaaat |
| 83761 | ctttctagat agtaaaagaa aaacctacca aagaaaacat gctacataga aaaacacagt |
| 83821 | aaatataaca aaatctacac aataataaat aacataaaga atagattaaa taagatacat |
| 83881 | ttttgggata tggatcaaac actacaccct cataatatgc cattttctca agcatacatg |
| 83941 | gaacattgac aaaagtcaat catatattga ttggcctcaa agaaaacact attaagttcc |
| 84001 | atttaagtac tttattacaa atagtgttcc ctcatcaaaa tacaataaaa ctagaaattt |
| 84061 | agaagaacag taagagaagg cttttcacct gaaattaaga aatcatcatg ttaaacaact |
| 84121 | gtagaaaaga gaaaaagaaa caaaaattat ctttaaaatt agtggtaata aaaatactat |
| 84181 | atatcagaat ctgtaaagga tacttaaagg cgtaattagt agtcttggcc ctccccttcc |
| 84241 | cttcccttcc cttcccttcc cttcccttcc cttcccttcc cttcccttcc cttcccttcc |
| 84301 | cttccccttc ccttcccctt cccttcccct tcccttccct tccccttccc ttcccttccc |
| 84361 | ttccccttcc cttccccttc cccttccctt tcccttcctt cttttcttct ttccttcctt |
| 84421 | cttctccatc tccctcctat ttttcctcct tccctttttt ttccaccctt tcttccttcc |
| 84481 | tttagcattt cttcaatcct tactttctcc caagcaaacc tggaatcttt acattgtaat |
| 84541 | attatttaaa agccaaataa aatcaaataa tatttaagaa aactatcaag aaatttcaaa |
| 84601 | tatttacaaa aatttattct cttgaacagc aagagaagga aacagtttag taagttagat |
| 84661 | aggcagaagt agacctagta cttgtgaaat gtgtgccaaa actcttcagt aactattttc |
| 84721 | cttctgcttt ctgtagaaaa tatttaaagt tgaaatgaaa gcatttcctg aagtgatacg |
| 84781 | tgaattacta caagcaatat gtacaaaaca tgaaaaacag aggtgatatt tagtttggaa |
| 84841 | ttgtcatatt caaaatcaga aataaaattt tcattctgat agagcagtga gtttgatttt |
| 84901 | attctcagat taaattacag aatggattat taatcagatg tgttttagat gattaaaata |
| 84961 | gcagttcatg tttttgatag cattagctat tcaggatcaa atgaatatct gattaaatat |
| 85021 | gtattttgag ttcaataatg ccttttacaa agcactUaa accctataga caaacgtggc |
| 85081 | ttgatggcag tgtagtaagg aatatttata ggtgattgaa aagctatatc taaagtttta |
| 85141 | actagtgagc tatcatcaaa gtgcatagaa ctttagaggc atattgccat actttgaact |
| 85201 | tgccaatgta tgtttcatca atactttact agtaatgtga atgaagaatt aaaggcttgc |
| 85261 | tcctcaactt ctgaatggct caaacatggt aaaataaagt tgacataatg aatgagaagt |
| 85321 | atagattaca gtagatgtga aaaatacaaa agaaccaaag aatgaaatta aagagacaaa |
| 85381 | tataaaatct aacatttgct tcaaYtgcat aagactttgt gggctactag tttgatagtg |
| 85441 | tttattttaa aaatattttt aaaattttat ttgaccttaa atttagcata aggcagtatt |
| 85501 | aaaaatcacc taataaaaat gatacagcct ccaattttat ttaaaaagtg tataatttat |
| 85561 | atcaattgag gaaactgatt atttagactg tgttcacagg agaatgacaa gtattttgag |
| 85621 | ggacagaaaa tcatatcttt ggaaaaagtt tgaaagcttg tcatagggca gatctattta |
| 85681 | gattgataac ataatgtatg gatgacaaaa ctaaaacatc taataaagag tacagggaag |
| 85741 | caggtattga ttcattataa aatcaaagtt ttataataat tagaatattt caaatgcaaa |
| 85801 | attttcatct tagaaaaagc aactggactt ggtgtaatgt taaaataatg catgtaaaac |
| 85861 | acatgatact tggtatatag taagcactaa ctaaatgcat actattaatc ttaccaatat |
| 85921 | aatgttctta gcattatatt caagaataaa ttagatcttt agttgttaca tataatcatt |
| 85981 | tattttagat taaatttaga gtaaatgtat ttcaatttgt ctaagcttct aagattcagt |
| 86041 | gattatattg cttattagtc atgctccatt gtatcatcca tccatatcct tttcatgact |
| 86101 | tgagagaatg caaatcttgt atcttctctc aaggaaaata ttatgactat ataattaaaa |
| 86161 | taattttaaa aatgtattta tattccagtt ttagtgctag tcctaatcaa aatttaaata |
| 86221 | atcattttac aataaatatt tgacatataa tattttgtaa tatattgaat tttctatagt |
| 86281 | agatgcctaa ttatcttagc tatttgcaaa taatgtaagt gtaaatatac tgataaatca |
| 86341 | agaaccttac tttaattatt gacataatta ttcaggatat taatctttta tcattgctat |
| 86401 | aaaacagtca attttcatat tgccattttc tttttattat Ktaaatacct ctctctgttg |
| 86461 | ggaatgcaga caaaaagatg gaagagattt ataataatta ggtaatataa gtttagtagg |
| 86521 | ggctccatct ttatttattc atatattcat agcaagtatt tattgaccat gaattctatg |
| 86581 | ccagaaaatg tgatatgtaa tttttttaaa agtcacaatc cttgctttca tggagcttat |
| 86641 | ggcctagtgg aggagtcaaa cattattgaa aagatcatat aaacatatgt aagattgcag |
| 86701 | ctgtcatagt ggctaggaaa gtgaggaatg taaacttctg atagtgtaaa caaattattg |
| 86761 | ttccaatcaa agagatagtg gatagcttcc ctgtgaaaat gaaaatcaag ctgacatata |
| 86821 | gatataaatg agtgtttact tgaagtttca ggagggtcaa tccagactgt gggaacagca |
| 86881 | tgtacaaaga ccatgcatca ggaagaactg ctggaaggga aaatggtaca gtatgaatat |
| 86341 | gttgaagcag gttggggata gcctacRtaa aggtgtgtag accatgtttt taatctttct |
| 87001 | cctaagagcc atgcaaattc attgaaaatt tttaaagtgt ggcaggagac aggatcagat |
| 87061 | ttgctctata gaaagactac tttggctaca atagggagac tgaagaggaa gaaaggtaga |
| 87121 | Yttcaggtag tcttacaagg agctggagcc taggaaaaca atgaaagctg gaaatagtaa |
| 87181 | agaaatgaat ggatattagg atgctcagga ggtaaaatca ataatgatag atttgatagg |
| 87241 | gggcataggc tggagaagat aatgaagcta acaagtttta tgacttatat aaaaagacac |
| 87301 | cattaaagca agagatgttg gaagaggact ggttgaatat cataagtatg atgttggcca |
| 87361 | tgttaaatgt taaatttgag gtatttttaa gtcatttagt agaaatgtca attatgtagt |
| 87421 | tgaataaatc agtctagatc ccagagctca gatacacatc tagaaataat acatttgtaa |
| 87481 | attgtgtgta gatggggaca atagcagtaa tgaacacaga tggaattgcc taggcagaag |
| 87541 | agctcaaatg agaaaaggat gtaggcccaa gtccagagaa ctgctaacat gagcttgaaa |
| 87601 | aaggaactga gaaagaggtg tatgttatgt catgaaattc aagggaaagt tgtgtttgtg |
| 87661 | atggagggag aaatcatcaa tgctaaatgt caagagacct gattagatga gctttgaagt |
| 87721 | atatccacta cgttcaacaa tacaataatt aagaagtact ttttccatga ggaatagggt |
| 87781 | atccaacaat atggggaaaa agagttttct cagagtgact gggatatata aaaatggaga |
| 87841 | gaaaatgtgg taatgtggag taaccagaga gtagagggaa gggctcaatc tctttgattt |
| 87901 | cattttaaca agaaacaaag aatcttttaa tgggaaatct agttaatagg atgatgttga |
| 87961 | atgcatgaga caggaactta agtatctgaa atagagagag attgatacat ttaatgatgg |
| 88021 | aaggttagcc ttaggaaagg ggacagctcc tgtattgtgg aagcagataa atagagtaga |
| 88081 | ctgggtatgg gctataggtt ggaacacaga aataagtatg atttttaatc tcccatcttc |
| 88141 | tatgttctct atataataac agtaggatta aagatgtgac aagtcgttgt aaaaagtgga |
| 88201 | caagtgagtt gaccaaataa ataaaaacct aagaagggta agaataaata attgtctttg |
| 88261 | attatttctg gttatgttgt ttattgatat ttaggaagtt ttagaactgt ctttctgaat |
| 88321 | tagtggtcct tggccttttt ttttttatta tactttaagt tttagggtac atgtgcacaa |
| 88381 | tgtgcaggtt agttacatat gtatacatgt gccatgttgg tgtgctgcac ccagtaactc |
| 88441 | gtcatttaac attagatata tctccaaatg ctatccctcc ccctcccccc accccacaac |
| 88501 | aggccccggt gtgtgatgtt cgccttcctg tgtccatgtg ttctcattgt tcaattccca |
| 88561 | cctatgagtg agaacatgcg gtgtttggtt ttctgtcctt gcttggcctt tttttaaatt |
| 88621 | ttgcttcagt gcagcacctc taatataagt aaggactacc acatactcat tatccattac |
| 88681 | agtgttctgc tgtatcattg attctgaagg atggagtgaa gcacctcttt aggaagacta |
| 88741 | tacaaaactc agagatggga aagagagaga gagagagaga gagagagaga gagagagaga |
| 88801 | gagagtgtgt gtatttgtta gaaaaagttc attatatgat tctgatatgc ctcccacagg |
| 88861 | gaggctagaa atttctcctt gaaaatagaa tagccaaatg ttgaaaatag accagaattt |
| 88921 | aggaatttgt ttatagatat atgacaagta attgttaaat ttaataaata gtgtagcaca |
| 88981 | tacaaataat cagctccctt ttcctaaata aaaaacttta gctattttta ttctatttta |
| 89041 | aaaattcact tcgtgatatt taaaggtatc cattattaaa atcagtaacc attccaattt |
| 89101 | ataatcattt aaaagtaaaa catataatta tttattagaa aaatatttgt gaacatttta |
| 89161 | taaatattat caacatgttg tcagtaaatt cctcctcagg tggaattttc tgactttggt |
| 89221 | tgagtcatta aatgttttgg gtcactagtg atcaattagt aaattactga tccatagtac |
| 89281 | tttaatggcc tgaaaaagta aatgcatctt ctgagcacct aattgaatct tagttattta |
| 89341 | ttcatcatta aaatcaagat aaatgagtca gctcacaata atgaaactga acagttacat |
| 89401 | tcatttcaca aatagattaa actaaagctc agaaaagacg ggacaaggga gttaaacatc |
| 89461 | caggttagat tttccatctg aaaagaccca attcccaata tcttgctata aaaattaaat |
| 89521 | acatatatga gtttgaaagc aatatttttc tgcaagttca ttttcatata gcgttattaa |
| 89581 | gtttaatacc attcagttaa taattgtaga tccatgRtac gtatactaat atggtcttgt |
| 89641 | tataatgtaa atagtacagt ttaaacagca aaatttcaca ctgttttaat ataaaaaatt |
| 89701 | tcctagaacc aattaccctg agtgcctact atatggttca tttggaaaat gaatgatagc |
| 89761 | tgcaatatgg taaaatgatt atgagcacag ccttcggaat aagtaaaaca tagctttaat |
| 89821 | atctaacttc tgtacttact aactgagatc tgaacaagtt acttaaactt ctaaggttta |
| 89881 | gtttctccac ttacgaaatg gagaggaaaa cacataattc atagttcatt gtaaggacta |
| 89941 | agtaaaaaaa aatacttaaa tacttagcac agggcctagc aacaaagata tatgaactcg |
| 90001 | gtacacacag acatacaaac acgtacccac actgcttaac tttagctaga atatttttaa |
| 90061 | aagcattatt agtttgagac tagattcatc caagttggtt tagcattcta ctagggatgt |
| 90121 | tacttttcat atctttcttt gtttttgttc ccaaaagcta taaacacaag ttttatcttc |
| 90181 | tttcctgaaa caattaaaaa gaaaatacaa gaaaatgttc aaaaaataat ttactagtgt |
| 90241 | ttttgtttgt ctggaagggt catcctgcag ctttaatcac agctgaagat cattatttca |
| 90301 | aaaaagacac atcttttaga tatttaaata tgtattatca tattatgcag tttaaatgtg |
| 90361 | aagtcatcta ccctctggtt tactgagatt ctgttacatt ttaaataaat agtaaatgct |
| 90421 | gtagttaaga gtgctatcac tgagaatcta agaagacaca ttagaaaggg attattactc |
| 90481 | caagaagaat tctctttcct taatcattac tttttcatta aaatatttct gaagtttata |
| 90541 | caatacaatc atagtagtga ccttaggcat atgacacctt agtgctataa aaactctaac |
| 90601 | aaatctgttt agagcccaat aatataaatt tctgtgaaga gaaatataaa ccaatggtaa |
| 90661 | aatgccatgt tgaagtttct aaatagccca cctagtaatt tacatgttaa gaacatattg |
| 90721 | aatatcttgg gaacatgttc ctagaaaaga ttctccatta taagcttttc ctttactgtt |
| 90781 | aatgattgct ttgataaaat gacccacgaa aaaagacaat tttctccctg tgttatctgt |
| 90841 | gtctgaaata ttcttcactg attttacata tgctgataat atgaacaagg taatttgtct |
| 90901 | gactctggtg gggcaagatt ttcagtaaga aatgcaaaat tcacacatag ctcttctgta |
| 90961 | agaagtctWt gcagtgtttc agtttatata aaaagattta tattgcctga ggcatctatt |
| 91021 | tttttttcct cgaggcaagc tgaagtggca caaatgaaat tgtttttcac actgttaatg |
| 91081 | tattcctttc tctttcaatt tctcagagag ccgacatagg gatttctgct ttaaccatca |
| 91141 | ctccagatcg tgaaaatgtg gtggacttta cgacacgtta catggactac tcagtggggg |
| 91201 | tactacttcg aagggctgaa aagacagtgg atatgtttgc ctgtcttgca ccatttgatc |
| 91261 | tctctctatg ggcttgcatt gctggcacag tccttctggt gggtctactg gtctacctct |
| 91321 | tgaactggct taatccccca cgattacaaa tgggatcaat gacgtctact actctctaca |
| 91381 | actccatgtg gtttgtgtat ggatcttttg tacaacaagg taaggagcaa aagtacattc |
| 91441 | tagtatttaa aaaaaataga atgtgatgtt tccaaagcca tgtattcagt taataaggtt |
| 91501 | ctgtatctga catcaataag tgttctaaag ggtactcaca gaaatgctca gaaaagaaca |
| 91561 | tttgctactt cctcctagtg gatctcagac acaccaagtt tccagcaata atggttcatt |
| 91621 | gtcttttgat gtaagtttaa tttcatcaac aaaggcaaat gtgaatgtca aaggtaaagc |
| 91681 | tcacagaaaa catcaagatc agattttcct ctccaaactc tgactttctg agaaatagcc |
| 91741 | ttataaggat gaaattactt attaccagag aaggtactat ttaattattt acatatgtaa |
| 91801 | gaaatgttag gacttgttct ttgttcagag acattatttc atgcattaaa aatattgtca |
| 91861 | ttgaaaagac tttggttaat ttgagactga tgagaaattt tgtgttttat cttttacttt |
| 91921 | gatatgtaat ctttactggt gatttaactt acaatttcac tattttctaa agaatgtcat |
| 91981 | tgctctgtta tttaatatcc aatctgccca actctttaaa cttgtccaat taattatttg |
| 92041 | gctctcactg tttcaaaata gaataatatt tattgcattc cttacttgag actattgcct |
| 92101 | catagagaat gaaatagtaa gaaaattgat agtctcttgc aagctctaca catatattga |
| 92161 | agaggaggat gtttgctaca atggagacat tatacttttg tgctaatatt tacaactctc |
| 92221 | aataacatct ttctttttat tcctattttc tcttggacct tccatttaac ttcatttttc |
| 92281 | tattctaatt taatcaaaga ttagtctaga accttcatct gacttgctaa cttctaatct |
| 92341 | agcccttaaa ccttttgtaa gcatgctttt ccctccaccc ttccatatcc agggctctaa |
| 92401 | gcttacctgt tactgtatat ttttcacaga cacttttagt tctttcatta ataattaatt |
| 92461 | cagtaaatat ttaatccaca ctatctaagt agaactttcc taagcaatta gtgtatgatg |
| 92521 | gtgagcaaga aggtctttgc catcattgcg ccacaacaga agtctaatga gacacaaaca |
| 92581 | tgtaaacaca ggattacaat tgtgggtaga aagttctgtg gtaggataag catagactgt |
| 92641 | taggagatca taggtgtaga agcacttaat atatgttaat gtcgcagaga aggcttttta |
| 92701 | aagtaacact ttatacagag agacagagtt aactatggaa agtgcttctt gaggaagatg |
| 92761 | aatggagaaa aggagacata aacggttagg atggaatttc agaccaagaa gtgagagaga |
| 92821 | aattaagata gtccaagcag agaggatcta gtaaagatga ggctgaagct acatttgtta |
| 92881 | ttataaggag tttgaactga tccttgaaat gatcagatat ttaaagataa taaatgggaa |
| 92941 | aatacagatt caaattttac tctagaaata ttctggttat aatgtggaga atagattgaa |
| 93001 | aaaaggcaat gtgaaaagtc agacaaatta gaagactatt acaataactg agcaaagagt |
| 93061 | tgatgtggct tgctcaaagg taaggtagct ttgggagtca agaggagagt attgatgaaa |
| 93121 | tggaatcaac aggctttgat tacctatagc gtggtgtggg ggtcagtact gaagaaaaaa |
| 93181 | ggagataaag ttttatccta ggtttttgaa tagaaaatta tgtatattgg atgtggatct |
| 93241 | catttactga aaaaataaaa cagaaaaaac agatatgaca ggagagatga tacattcatt |
| 93301 | ctgctgcaca ttgagtttga gctatccgtg aaacatccaa aagagtatta ctaatgtgtg |
| 93361 | cttagtggtt tgatatattt gtcagatgca cagaagagac atctggactt aatatatatt |
| 93421 | taggagatat tggaatgtag gaaattttga agacatggga gttaatgaga tcacacagag |
| 93481 | tagttgctct taattgcaaa ctcctgattg aggaccactg gcccatacaa agcctaggac |
| 93541 | agagctctaa ggaaaggaat ggatagagga gcactcagtg gtgcctgata aggagtgact |
| 93601 | ataaatgcac aagtgaaaat cagagcagct agtagtatac gtttggttga ctgtgttaaa |
| 93661 | cctttcaaag aatcaaatga aattaaggct tgaaaggttt ttgttggatt taacaataaa |
| 93721 | tttattattg aagcaaaaaa ggcttgtctt tgccctatga tgtctgaggt ttcagctaga |
| 93781 | caattcagag gccagaggct aaaatcatcc aaaggcttgt tcatttcact cacatgtctg |
| 93841 | gtggttgatg cggtctgtgg gctagtggcc tcagcttttt ctccatttgg gcctctccat |
| 93901 | gaggtcactt tgcatgggat agtttgactt tctcacagca tggtagttgc tttctccaca |
| 93961 | gagaacatcc caattaaaga attttatgtt ccaggttggg agtcataaac ttgtgtgatg |
| 94021 | gtgtgaattc ctcttgcaga gaacatcctg gatgtaccca cctcatgggt ttttatgaga |
| 94081 | atttattaat gaatggttaa aattgtgaag ttcttagata taacttatat aagtgtcaca |
| 94141 | taagtgttta ctaaataaat aaaacaatga attagactca gcggcagata attgacatat |
| 94201 | gtaaaaaaag ggataacaag acaaatgatt gtagatcatt ttgatgtaSa tattgattga |
| 94261 | aatagtagat catggggtat atggagaact agaaagagaa ggaccaagaa tgcgaatgta |
| 94321 | aagaagagta agggtagaaa ttgatagagt aggcctggca tggtggcaca tgccagcact |
| 94381 | ttgggaggcc gaggcaggcg gatcacctga ggtcaggagt tcgagaccag cccggccaac |
| 94441 | atggtgaaac accatctctg ctaaaaatac aaaaaaatgc caggcatggt ggtgggcgcc |
| 94501 | tgtaatccca gctactcagg aggctgaggc tggagaattg cttgaaccca ggagcgggag |
| 94561 | gttgcagtga gccgagattg tgccattgca ctccagccgg gatgacaaga gtgaaactcc |
| 94621 | atctcaaaaa aaaaaaaaaa aaaaaaaaaa aagatagagt agttaggaga gagtagtaga |
| 94681 | ggtctcaata agattacaga aaaagagtga aaacataatt ataaatgagt ttggagtctg |
| 94741 | tatttataaa ataggacatc taagcctaat tttttttgaa atgtaataga ggagaacatg |
| 94801 | ttgtaggtat gtggatacaa attccagatg ttaagatttg gtgaaggact tcaaagctga |
| 94861 | tcaaggaact gagagataaa gggttaggtg agccagctgt aatgatggct tggattagac |
| 94921 | tggagaaaaa gaaattgagc catgtgtcaa agttgtgaat aaataaggaa ggatgacaag |
| 94981 | gatgttgata gatatgagca ttgaccagaa ataggaggtg acatacctag atataattaa |
| 95041 | cctctgtttt catcttactt cttccaattt attcagtttt tcagtgtatg atttcaacaa |
| 95101 | ctgccaatat gctgatgact cccagatcta catcatctat tcttgaactt cagatttgta |
| 95161 | tattcaactg ttacctaggc aactctctga ctataccaga ggcaccataa atttgaacat |
| 95221 | gccaaaaatg taattcattt tagaatttga accttcttag cctctcacat tctctattcc |
| 95281 | agctgatcca ccaccaacca ttcagactca tgttagaaac tctgaaaata gttttcattt |
| 95341 | cttcctcttt ctcatcttgt atatccaacc ataatcaagt cctgtcaatt ttacctacta |
| 95401 | aatatttcta gaagtggaaa tctcttctta tctagaaaga ttagcctaga ttgtcaccta |
| 95461 | gattgaggct acctcatttt tcacctgaac tataggagtc tcataagtct ttcttcttcc |
| 95521 | agtgttgtct cctgcaaata tacgattcac atacttgcat atatacccaa gttatccatg |
| 95581 | ccatggaaag ataaacacat aattcccatt cttcagttat tttcttggct tcccaatgcc |
| 95641 | cacagtttag aagtctgagt tcttcagagc ataaacaata ttccatcacc tggctcctgt |
| 95701 | ccaacactca gttctcatct cttaccattc gtgcctcaca tttaattgcc cagttactcc |
| 95761 | aaacttacgg tttgaccata agacaagact ctttcctaac tcttcacctt Kgtatctgct |
| 95821 | ctttccaatt ccttgagtgc ctttgttacc aacctgatta gactctactc ccactctctg |
| 95881 | tctttataca gcaagttcct attcatcctc taattttttg cttttatttc tctactttca |
| 95941 | agaagtcttt cctgggattg agaaccatct tctatactcc tcaacatgta tttgtcatag |
| 96001 | tacttaccaa ccatagtgta attccttttt ttaaacatgt ctccacctga gtcctttgaa |
| 96061 | tataagatag tgtactattt aatctgtgta gtctctttgc ttatagttaa gtgccttgta |
| 96121 | catgttaggc atttaataaa ttcatattga taaatttaat gaatggatgc taaatactta |
| 96181 | actaaataaa gcatggaggt tagctgcaat attttgttta ttagattata gtctcaaata |
| 96241 | aaacactaaa attatccaca aatgacttaa aactcactaa accaaaaaga aatttatctg |
| 96301 | taatgtcccc actgagttgt tgtaagaaac taagaggagt ttataagtaa ggcatcctct |
| 96361 | tggttgaagc ctcatttgtg aagacgttga catacttatg aattggcttt ctgaattctt |
| 96421 | cctccaagtg accaaatttc aataagtttt ggagcacacc caggaaaaaa gataaataga |
| 96481 | attacccata tggttagcat gcttttctag ttctataaac atgcaacaga taacaaaagt |
| 96541 | tataaatatg ttaaatacct ttctatccca ctgctccctt aaatgagttg cacagcctgc |
| 96601 | aggttgattg tacatttata caaatttata atgacagata atgctgaatc gcatatgatt |
| 96661 | agctacttca tctttgaaaa gctgctattM ttccacttgc taacaatgaa aaggtgaatt |
| 96721 | cattaaaaga Yactgagagc atctttatgc tctgattaaa aataaatgaa gaaaaggctt |
| 96781 | ataaacccta tgaaaaagag aataaatcaa tgacagtgtc catgtatagg caattgactt |
| 96841 | ggaactggtg atatttccaa taaaaataca gtcttttaag aggaagtaca gctgttattt |
| 96901 | cagaattgca gtcttcattg agccactata ccagattgaa tagggataaa gaggcatttc |
| 96961 | atgtgttatc agacagattt ctaaaaaaag agttcacgga cagccaaaag cagaaaaatg |
| 97021 | ttaaatatat gtgagttgtt tactaatcct gacattttaa aaaatctgta acttcaaagc |
| 97081 | attctggaaa aatagctcag ggaatccaac tgtgcaaaag cagtatggtc cattatagag |
| 97141 | ataaactcag tacatctgtt tggctggaac attaagagct tgtgaattag gttgtaaaat |
| 97201 | gcacgaatga gagaccaatt ttatacaact tttgttgtca ttttatgtcc tccgttatct |
| 97261 | ccatgtgtac atacatctcc tttcaattaa tattcttaag tgaaactgga tttgattttt |
| 97321 | agaagtctct tatgtttagt ttattggcag tgactctatc atctggtctc cagaggactt |
| 97381 | caatgacttt gcaaatgtag ttgtatgtaa tttKctttct gtttacatgc aaagggaaat |
| 97441 | ttcagaatta tcttcagatg tttgtgtctg attatgggaa atagaaatat ttatagtcat |
| 97501 | gtaaaatatt tgaagcaacc acatatatgt ttatttagca gcaagcattt attaagcacc |
| 97561 | aacaatgtgc caggaacatg agtagcaaaa ggaaaataag gaataactct taactagggt |
| 97621 | ttaataaata tttcacgagt atactatgtg |

Following is a genomic nucleotide sequence of a PDE4D region (SEQ D NO: 4).

| >5:58309301-58408050 | |
|---|---|
| 1 | ctctaaatat tttccattga tgaatttcta attgtttttg gcctaagtag tctgaaaaca |
| 61 | atacacagat ttagggttgc aactaaaaaa acgggaggat ttctaagtga atgcttttga |
| 121 | atcctttgtt ctcattatac atcagaatgt ctgtgtacag cagttaaggc ataaaggaaa |
| 181 | gtaaaaatgc agttgaatct caatactgta ttttgctttt cagaactgca attcttcaat |
| 241 | ttacattaSg catttaagat tgttcaggaa gcaaactaaa ataataaaac aaaatgtcct |
| 301 | taaatgtaat ctcctttact tctacatcac gccagtgtgc tactgggtat attatatatc |
| 361 | ctcccaatat atatttaagg aggtggtatg ggtccaatag taagaaaatt ttcttatttt |
| 421 | ttcctgacaa tattctgcta aaaatcattt gaatatgctg cactgtgatg cattacaagt |
| 481 | ttggtagata tcagtattca aagttattta aacaaatact ccattgatgg tataaaaata |
| 541 | aaWccttcaa aatgcataag accaaatata tctatatttg ctatttttac atgtttttaa |
| 601 | aggtacagta ttaaacataa agctgtccag gaaaaatttt cctcaaaaag aaacgtacta |
| 661 | aaattctata tgtagattct tggcaatatt gttcataatc atagtacaaa tggttcataa |
| 721 | tcacagtaca aatgtgaagc ccttcaaaaa taacatcagt tgtgagctgg ctaaaagcta |
| 781 | ggagctgaat tctggcggat aatgagggct tcttatattt actctctata cttttctgca |
| 841 | tttctgaaga tttttagcca agaaatggct aacaaaattc aaataatgga agtctatata |
| 901 | ttttaaaaac atttttaaaa atctgaatca tccacagatt taaagggagg taaggaatca |
| 961 | tgactacttg gaYaaccaag actgaaataa gttttctaat ttttttagaa ttcaagtaaa |
| 1021 | tctctaaaac aaaggggaac ttgtccttca aaaagaaaga aacaaagcaa acgacRaaca |
| 1081 | ctccaatctg aaaaatcctc agagtaacag aaaatgaatc ttttaaaatg tgttgtgatt |
| 1141 | ggctcccagg ttcaagcgag tggctcaaat accaaaaatc tgtgaagtga tgtttcattt |
| 1201 | aggactgaat aaggtttttc tacctccttc tattgtggag acaggatcac aatgctaatg |
| 1261 | ttaccacagg gcatgSaaaa agcctgccaa gcatggacac aatcttattg aattgtttat |
| 1321 | taattcattt cttgagattc acaaaacagt gtaataaaaa ctttgttcag ttttgcaagt |
| 1381 | ataatagaca atcaagtttt cactttagta tcacttacat agttaagtaa tgtcaatttt |
| 1441 | taatgtcacc tagggcttta tagctaaagg tgaaaaaaac gtattcctcg tcaggttaaa |
| 1501 | gtagtataga tttttagaaa cagccacaaa ttaattttat aaactttatg tgcaacatac |
| 1561 | taactggaat ccataaatca gtataagtaa tttggcccRa taatttattt gcaatgaatg |
| 1621 | tatttaatta ccatatattt ttcaagcaca attcaaacat ataccacatg acaaacagaa |
| 1681 | gaactactgc gtatctaaaa aagctatttt aacactcaaa ctacaactga tgaaagttat |
| 1741 | acaatcttat cttgaccact attaacacgt atttcttcct agttcatgtg taaggaaaga |
| 1801 | atacataact gatagtttga acaaattaca gtagagtcta agctttctaa ataattttgt |
| 1861 | aatcagagta aggagttttc aaaagctaag taagtcttac ctttatgtag ccccaagaca |
| 1921 | ctgacagtaa atagtttgct tcaggcattt ttgattgatt atatgtatat atatatatat |
| 1981 | gtatatatat agtaagctct aaaatgtatt tagcttcaaa atctcgacac tttaacaacc |
| 2041 | tccacaagcc acgcagagta tgagttttta aagatttttg gtaattaagt atacataaga |
| 2101 | aagcctcgca ttaaatccca tccagccgtc ttcccatcga atacatgcca tttgtaagac |
| 2161 | tccagaatgg cacaaaattc agcaactatt ctgaataaag aaaatccacg tgataaagga |
| 2221 | atatctaaag atctaagggt ctgcatcttt ctctcccgag ctccagggct taatgaataa |
| 2281 | tgtatgtcaa gatgcttagt tgcaagaaag gctagtcctg tcaaaagcta ccaaataagg |
| 2341 | aactgcaggg aagccgggaa gagtgacaag aagcccctcc cataaaaccc caactctgca |
| 2401 | ccaattgcat tccagctctt agcctataag gcgagggggt gtggcctcgg tgcaggctgc |
| 2461 | tttgggattg gttggcaagg gccagcctct ggggaggctt tctcctcacc cgacaaacgc |
| 2521 | cattgaataa tggtatccct gcagctctga ggccaccctg agaaaagatg aacggggctt |
| 2581 | ccctcttgca gtgatgaata atgaccataa aactcctcaa aattcgtagt tcattcttaa |
| 2641 | gaattgccca tccttttgga gggcattttc tatgaatttt aatgtcctct ggcttccctt |
| 2701 | ccttccaaat aaaaactgct ccaaaaatgg caatcgagca ttttaattgc ttccYtctct |
| 2761 | caaatccaaa acaagtggct ccctggtgat gtagaaatga ctgtagttct cccctcatcc |
| 2821 | aacataaaag catagggatt aaacaatacg actaatgtcc agaattcaca gatggcacag |
| 2881 | atggcaatga cacattccct cacctgtgtg Scatggctgc tctcagctcc ttttgtttct |
| 2941 | attgttatgt atgacagagt tctaaaacct tcaaaatcta ggggtgggac cacttgcaat |
| 3001 | tctgggggga gggaaatgtg aaagctaggg cactgtgtat gcataaggaa tgcagtgttt |
| 3061 | tgtatataga actcaggatc tgtattttga tcagaagaga atattttatt tatttattta |
| 3121 | gagacagagt ctcgctgtcg cccaggctag agtgcagtag tgcgatcttg gctcactgtc |
| 3181 | acctctgcct cccgggttca agcgattctc ctgcctcagc ctcccgagaa gctgggatta |
| 3241 | caggcgccca ccaccacgcc tggctaattt ttgtattttt agcagagacg ggatttcacc |
| 3301 | atgttggcca ggctggtctc aaactcctga cctcagggga tctgcccacc tcagcctccc |
| 3361 | aaagtgctgg gattacaggt gtgagccaac gtgcccagcc agaacagaac attttaataa |
| 3421 | ctgtacacaa agttaagaaa ggaatgagtg gctcttagga atccagaatt gagctcaaga |
| 3481 | gtaatcagac attttacgag gagggttaaa tgagaaatca aagttctttc tagaaaaaca |
| 3541 | gcattttcta tttggtttgg tgtagtatta ccctgtaact ttaatactaa agaggtcttg |
| 3601 | tgggtgagaa acaaggaaaa actaggtttt agttgatttt agcatataat tctgtgcatt |
| 3661 | ttacctcctc tccatctcaa agcctgtgct tactactctg tgctttaact tgctggtatg |
| 3721 | gctactctgc agtcttcctg acatttaact atttccacat gtggcatgtc tgtcttagag |
| 3781 | agcatgctct ctgataagca ctcctgtaac actgtgtctc tctgagtttc tattggcctt |
| 3841 | tagctgctac agtcctctgc ttttgttaac atctgttgta ttcatttcca tcactcttac |
| 3901 | tcctctacaa aaggaagaaa ttaaagctcc aacattccac taaagctacc cccagaggaa |
| 3961 | tactaaggtc ctccttttct ctggtctccc tacttctctg aagtgttcca catgtttgat |
| 4021 | cattcctact actctccctc aactcccagg atcctgaact tctttaattt tccctaggcc |
| 4081 | cacaatcttc aatgaccact tctaacttga acttcagtgc catatttctg aatacaagat |
| 4141 | tctcctctcg aatgccctgc tagtcacgac aaactttttc aaaactgtga gacatttttc |
| 4201 | tatttaaagt agttcttagt ataattactt ccatttcagt cagtgagcct atatttcttc |
| 4261 | cagtatctca gatggaatct atgactcttt cctaccattc aacaggtgtt tcatgtgcgt |
| 4321 | cagacactgt caggtgttta atataaacca tctagctgaa tccttgagta acccggcatg |
| 4381 | ttcccctata cggcatcacc ttccctcctt cattcaactt tgttagcaag ccctatcttt |
| 4441 | tatccagagc tgtttctttc agtttRttcc ttaccattac ccctcccgta atctgaatcc |
| 4501 | aaatatttaa aagactgaga gaaatagaaa gtaactttaa tatcaccccc tctaactccc |
| 4561 | tcatttccac ataaagttga gacctagaga gaaaaaatga ttttcccagg ttcactctgg |
| 4621 | tactgagaac aagaattgca agtttaaatc ttttgtgcac aatcactgac aaataatctt |
| 4681 | tttagtgtgt cagcttcctc atctgtaaag tggattactg tggcagctag tctccaaaga |
| 4741 | tactcttaat gagccacact gtcctacact gaatctgagt tggcttgtgt aaccaacaag |
| 4801 | tgatggtgca tgactgctga ggctaagtca taagaaatct tgcagctctc atcctgcttt |
| 4861 | ctttgaatgt gctattggga cccctaagcc attaggttga aaaaaaaaaa ggtctgacta |
| 4921 | ctctgagagg ctatgctaca agaagtccat gttaacttca cagacagtga agagagaaag |
| 4981 | cgcaagagag cacttgcaca agaaagagca cacatgcctg gccagtcttc agcaatgcca |
| 5041 | gccaccccag cccagagcca gacctgaggg gcaagaagcc atcctagcca tccagccagt |
| 5101 | tcagctttca gatagctacc cactgggctg caaccatatg aggaatgcca agcaagaatg |
| 5161 | tctagccaag accagttaac ccacaaagcc atgaaaacta ataaaaatac attcatattt |
| 5221 | caaagtatta ggtttgaagt gatttcttac atagcaataa ataaccagaa cagatacata |
| 5281 | tgaggattaa atgagaccat atatgcaaag cacttaaaac aacaaatggt acaaagttga |
| 5341 | agataagtag gtatcagaca gtaaagatga cagtgacaat gatcattatg ataatcattc |
| 5401 | tattatacta atatttaaaa aaattctctg gtttgtcatt tgtttttaaa atatatatgt |
| 5461 | tatatttcct ttcaattgca tcacagcact ccatgtctca gggccaaaat gctgtaattg |
| 5521 | cttcttagct catcttcttg cccctggctt ttccccttct agtctcatat tgacctctac |
| 5581 | tgccagatca attttcctca aatccagccg cagcctgtta cttcaaagct agtaatgcct |
| 5641 | gcttcatcac atccctctgt aaattccaga ctgccatatc tgaccttaat ccccaacgcc |
| 5701 | aagcctattt tcagctgctc tattacattt gctctgttcc tgaaagacta gtaYtcctcc |
| 5761 | aYcttcacca tacagctgac aatcccatct ctgtcttacc aaattctcgg cctaaaatac |
| 5821 | cctccttctt ctactctgcc aacctcaaac tggttttctc agtcagattt aaatcattca |
| 5881 | aagtacatct taggaccaaa tattccagcc tttttccaaa ttctaacgat tctacatctg |
| 5941 | ccccttcatt caatatttat taagctYcta cYctatgtca ggcactgcct gagaaagaga |
| 6001 | aacaaagctt tctcacctgc agcttatatt ccaaaactaa gttaataaat acataatacg |
| 6061 | tcatggggtg ataagcacaa tggagagaaa gaagggataa ggattacgga tagaggttgc |
| 6121 | tataaggtga tcatggaaag cccctctgat gagatgacgt ataccaggag atctgaagaa |
| 6181 | aggaaacaag ccatgtggat tgcggggtga gaggtagggt agagaaggga aaaccaagtg |
| 6241 | aggaatggca aaagcaaagg cctcgaggca aaacatactt ggtgaggctc cctgaagaac |
| 6301 | aggtggaagt gtcataccac ttgtcattta aatgcaaatt acactctgca gtcaggctca |
| 6361 | ctgccttccc cctcctttaa aaaaaaaaaN atccatccat ccatcaattt tattctttga |
| 6421 | caaacttaag actcagttac tttttacttt ttataacaag tattcatcct gaaatgaaat |
| 6481 | tcctaaatac acttccttca taatactgta ctgaatacat ataggctaaa tactccctac |
| 6541 | atagatttaa aaaaaaaatc agtataatgg cttatcagtg aaataaagga gaaatgagta |
| 6601 | atttctatca aatactacaa aatataaata cttgtacaca actacatttt aaaaagtagt |
| 6661 | caaaaactac tataactgat tagctgtgtg accttgggga agctatttaa atttcccata |
| 6721 | cttcagttta ctcacatgga aaatgagaat aatagtttct accttacagg gttgttgtga |
| 6781 | ggattaaatt agtgcatata cataaggctc ttagaacagt aagtgctatc taagggtttg |
| 6841 | ctatgatttc tcttatgggg agaattacca caaaagtaac agtagcatga aaatgaatac |
| 6901 | aggggtgtta tgttagaaaa attttccttt ttcctgatat gatgaatgat tctttgaaag |
| 6961 | gtttcaaatg taacaaagtg ccaYcttcac aatatacact attgctgcat tactagaaaa |
| 7021 | tttggtgtgg attaaaacca tgtaaaaata tttaatactt atacgtaaaa agtagttggg |
| 7081 | ttctaggctc agagaattat aaatgcattt tgttttttca cctatgtgaa tagctgctgg |
| 7141 | gactttttgg aagttgtgtg ggacacacag caaacctccc tggtgaaagg ctgtcccatg |
| 7201 | tatcccagga catctagcct ccctggcctc tgcYcaccaa gcaccaggag tactccccag |
| 7261 | tcactgtccc ctgaaatttc ctgaatgttc cccagtgtta agaatcactg ttctattttt |
| 7321 | ttcaactgtc ttcaactttg actcctcatc cagggtggag gatctttgag gccagaaaag |
| 7381 | tatcttatta tgtccctgta tttctcaagg tagtgtacta ttgccacaaa aagattgttg |
| 7441 | gctgatgcag atgcaaaaca tcaatatttt tatactgatt ataggtgaca ggaaaggaca |
| 7501 | agggttttca ccctcagcaa aatcatcaaa aacatttaaa acagattaca tatagtagat |
| 7561 | aatactcctt gtcttaaaac attatgcaga aacacacata cagtttccct tccctctaaa |
| 7621 | atctgcagcc tcttctccct gctgaatgtg agtgatggtc ccttcattca acagacttct |
| 7681 | atttggcaac tattatgtgc caggcattct aataggtgct ggatgtgaaa ggatttaact |
| 7741 | tattcaaata agtccctaat ttgctgcaat aagggagatt atttaatcat attgagtaaa |
| 7801 | aagtaccaag gaaagtttcc atatttctag ggaataagaa atcttttcaa aaatctctga |
| 7861 | gccaaattaa aattcatttc attttcccta atctccattt ctaggagact agaatctcct |
| 7921 | tcctattgcc acggtagctg ctatctgcag agagaatcta gataatttag aagagaaatt |
| 7981 | tgaatagagc cagagagata caattcttat agtcagactt ggcttcacca aatttaatga |
| 8041 | agaaacttta ataagttaca acatttacta ggcacctact atgcacaaag aactttacat |
| 8101 | acctcatttc taatcttggc aacattctga taagataaat attatcccta ttttaacatg |
| 8161 | aaaagatgga ccttcttaca tattcaataa cttgcKccat aggtgacagg attgaaatgc |
| 8221 | aaacccagtc cttgctctta atggtgtatg acagctcatc atataattag gctgatgcaa |
| 8281 | actctatgat aaacagtatg aatctaggag tggatttgtt tatcctcaat ctttttgcta |
| 8341 | actttacagt gccttgaatt aaagaatcRt ggttactatc accatggtca gatctctgga |
| 8401 | cagggtgagg aagcaggtgc ccacaggcag acagccctgc catatagtga gacacgctta |
| 8461 | tgtttgggag cgctatgtga acaacacaca gctgccctca tatgcagatg taatgatgac |
| 8521 | gacacattca gacaaggaac tgaggtccag aaaagttctg acaacacaac ttgagggaca |
| 8581 | gcgttacagc tagagctcag gtttcttatc tgtaatccac tgctctcaaa tgcccaaatg |
| 8641 | attttatata catatatgca agtgtgtttg ttgaaataga aaaaatagtt tcttggcctg |
| 8701 | gtgaagtgcc tcatgcctgt aatcccaaca ttttgggagg ccaatgcggg aggattgctt |
| 8761 | gaggccagga ggtcgagacc agcctgggca acatagagaa accaccgtct ctacaaaaaa |
| 8821 | tttaaaaatt agccaggtgt ggtggcatgt gcttgtagtt ccagatacta ctgaggtggg |
| 8881 | aggtgggagg tgctgaggtg ggaggatggc ttgaggccag gagtttaagg ctgcagtgag |
| 8941 | ctatgattgc accactgtac tccagcctgg atgacagagc aagaccttgt ctcaaaaaag |
| 9001 | aaaaaaaaaa ttcccataat gttcagttag ttctcttttt cagacttttc ctagaaatac |
| 9061 | ccctcagaat ttctagaggc cacttccctg ctattccttg ttttctcagt ccttaccaga |
| 9121 | tacccaagca attaacaatc tttttgaact atttcagtac atttcaggat atttgaaatt |
| 9181 | ctgctgatgg agctgaactg ctgatacatt aacacatgct gtcattattg actttgtaac |
| 9241 | agaggtcaag agagaaaaga tgcatttttg aatagggaaa aacaaaacta aattatgggg |
| 9301 | aaactaaggc tcagagaggt caagttgtct tgctcaaggt cacaaagcca gtagtaagta |
| 9361 | acagggccag gatttgaacc cagactgtct ggtttcagag tactctaaat tgccaaaaaa |
| 9421 | agaaaactat ttcaggcatt attttatatt tagcctagag cctacataat agatccgtta |
| 9481 | atataatcaa ttctaagctg tatattatat tactgtgttt gaaatattta agaaatattg |
| 9541 | tttaaaataa gtgtattagc tcttRgacct ctccaacata aggagttttc caggcagtag |
| 9601 | cactgtttgc agtaggactt acattattgg aaaatatttt atcctataat caatctacat |
| 9661 | ttattttaca cctttcacct taaaattaaa agtcgtggta gttattaatc aagaagcaaa |
| 9721 | tctatataac gtggtctctc atatatccag tactgttcag cttgaacaaa tgcaaatcta |
| 9781 | atttttaaat gtcccttgaa gaattctgag gcaaagttaa acttcaagta ttaaacagaa |
| 9841 | tagccacaga aaactagtgt ttctcagcct ccctttgctt tcaaatgcct taaaaatatt |
| 9901 | ttagaggcag acaataatct agaattgatt cataaccaat attttacata acagaatgtt |
| 9961 | acatgtattt tcatattata aaaactatac tttctttgat taatgctagt attctgaaaa |
| 10021 | catcgttagt gccagtaaat ccactaaatc tgtttgtatt tactgccctc tagtgataat |
| 10081 | ggctctacat ttgattaaga aaaaactatt cctttcagta aatgaaaaca tctcttgtaa |
| 10141 | gttttatgga gatttgagat taagaggaac tccaggctgc ttttttaaaa ccaaataatc |
| 10201 | atctgaacaa gctgagaatc gttctctaaa tgatcttatt ttaatttttt tttaacaaaa |
| 10261 | aagtctcatt taaaatagta aacacctgca acatgtatgg agaaaggaaa ccatttttgc |
| 10321 | taagaggctc cttgaagctg aaataacatg agactgatct gatagctgta tttatatttc |
| 10381 | tcttctacat gggctaactc tcttattaga tgttagcata gtattctgta attgttgata |
| 10441 | cagtttgaat tcatatatgg tagagacaga gttttgtata actatttagt caatttacct |
| 10501 | aaaaccagca gcagcagcat atctactaca gacttttgtt tcactgaaga ataatcatga |
| 10561 | caataaatgg gtgcaaaaac ttcacataaa tgaagcctcc acaaaaataa tcttgtatga |
| 10621 | aaaagtattg tctccttata gagaaaatta ggatctccaa ttcacttaaa ttggcctaaa |
| 10681 | tgaataccat aatcatcata aaatagttac taatgtcccc tctgtacttc gtggtatgct |
| 10741 | ttgtggaaac aatttaaaca aagagaatat ctgtctccta aagtaataat aactagtgta |
| 10801 | aataggcatg cttccagaaa tatataatct tttacacata tcactttaat atgtgcatct |
| 10861 | taaaataatt ttaagtaata aaatttgagc tataaaagag aaatgatatt cttttcaaca |
| 10921 | aagaatgctt gaacaattga gcatccactc ataaaacaag aaacttcaac ctttatcctg |
| 10981 | caccacacta acatctactt gaaatgaatc atagaactaa atgtaaaatc taaaactaaa |
| 11041 | gaacttctag aagaaaacac agtagaaagt agttgtgatc ttgYgttagg caaagatttc |
| 11101 | ttaaaccaca aaagcatgaa gcataaaata aaaaatggta tactggactt taYcaaattt |
| 11161 | ctctttgaaa aaaactgtta agaaaaYaga acaacaagcc acagacttga gaagggaata |
| 11221 | tgtacaaaat gcatatcaaa taaaggactt gtatccagaa attatttttt aaaaaatctt |
| 11281 | tacaacacaR taagaggaca atccagtgaa aagatggtca aaagatttga acaaacaaac |
| 11341 | acttcataag aatcaaaaag atcaataagc atgtgaaaag atccaYgacc atcattagac |
| 11401 | attagggaaa caaaaagtaa aaccacagtg agatgccaca Ytacatactt actagaatgt |
| 11461 | ataaaataca aaatacaaac aatacaaaat gctggcctgg atgcagagca aattaaattc |
| 11521 | tcatacattg ctagtgagaa tgcaaaatgg tatagaaact ttggaaaaag gtttgacagt |
| 11581 | ttcttataaa gttaaaaagt agagtgacca tatgacccag caatcctact tctaagtatc |
| 11641 | tacccatgag aaatgaaaac atatgtctat ataaagacct atatgaaaat gtttatagca |
| 11701 | actctctata gcagaaaccc agaaacagtt cagatatcag tcaactggtg aatgggtaaa |
| 11761 | caaattgtgt ccataaaatg acatactgga aaaaaatgaa atactaaaaa gcaataaaat |
| 11821 | gaaacaaatt accaatagat gcaataacac aaaatgtatt ataagtgaaa cttataattc |
| 11881 | aactcaaaac tcaaaagtat tgtaagtgaa agaagccatt cacaaaaggc tatatcttta |
| 11941 | atgtattatt cattctggca aggcaaaacc atagggacaa tcagcactgg acgtggggtt |
| 12001 | tggggaatga ctataaaaag caagagaaaa ctttttgggt ggtataaata tcctatatta |
| 12061 | tatagtacat atatattata attacacatt acctatatat ctagattatg gtggtggtta |
| 12121 | tccaattgta cacatttgtg aaaatgcatt caactatacc tctagaaaga gtaactctta |
| 12181 | tgtaaattat acctcaaaaa tctaactttg aaaaatatta gctgatgtta tcaaattatt |
| 12241 | tcaattccag atttgcttaa ctttacaact ttgcccaaag ataaatgcaa aacaaaaacc |
| 12301 | tcaggcatag ttatgtatga taaaacacca tttttaccct ttcaaagagc tccaccatta |
| 12361 | tttcccagaa aaYaggcata cagttatcca aggtccacat tagctttaat gatacaacat |
| 12421 | tcagtgaatt cccagttgtg aaaagccttt aggttggcag ggcgctgtgg ctcatgcctg |
| 12481 | taatcctagc actttaggag gccgaggcag gcagattgcc tgagctcagg agtttgagat |
| 12541 | cagcctcggc aatatggtga aaccctgtct ctactaaaaa tacaaaaaat tagcagggtg |
| 12601 | tKgtggtgca ctcatgtaat cccagctact cgggaggctg aggcaggaga attgcttgag |
| 12661 | cccgggaggc agaagttgca gtgagctgag atcgcgccac tgcactccag ccggggtgac |
| 12721 | agagattctg tctccaaaaa taaaataata agagaagcct ttaggtgata aaataatagt |
| 12781 | caaagtattc ttttaagtat tattatttta ttttttttta attctttgta ataaaaagtt |
| 12841 | ccataaagta aaatattgtc cttttttgtt ttatttttat ttgtatcaat tcaaggggac |
| 12901 | aggtgcagtt tgttacctgg acctgatttt taaataaatc aaactcagtt tagttatttc |
| 12961 | atggtgatcc tgggagactg tttgtcagtc atcatcgaat agcgaatgag tatcctttgt |
| 13021 | atgctcattc taggctgtaa gtaccatgtc agctgatgga gacacaaaaa tccaataaag |
| 13081 | taggtatagc tgggggaggt tccaagatgg ccaaatagga acagctccag tctactgctc |
| 13141 | ccagcatgag tgatgcagaa gacgggtgat ttctgcattt ccaactgagc tttgaaaaga |
| 13201 | gtagtggatc tcccagcatg aagtttgaga tctgagaatg gacacactgc ttcctcaagc |
| 13261 | gggtccctga cccccgagta gcctaactgg gaggcacctc ccagtagggg ccaactgaca |
| 13321 | cctcatacag ccagatgccc ctctgagatg aagcttccag aggaaggatc aggtagctac |
| 13381 | atttgccgtt ctgcaatatt tgcggttcgg cagcctctgc tggtgatacc caggcaaaca |
| 13441 | gggtctggag tggacctcca gcaaactcga acagacccac agctgagggt cctgactgtt |
| 13501 | agaaggaaaa ctaacaaaca gaaagggcat ccacaccaaa accccatatg tacgtcacca |
| 13561 | tcatcaaaga ccaaaggtag ataaaaccac aaagatgggg agaaaccaga gcagaaaagc |
| 13621 | tgaaagttct aaaaatcaga gctcctcttc tcctccaaag gaatgcagct cctcaccagc |
| 13681 | aacggaacaa agctggacag agaatgactt tgacaagctg agagaagaag gcttcagacg |
| 13741 | atgggtaata aacttttccg agStaaagga ggatgttcaa acccatcgca aagaagctaa |
| 13801 | aaaccttgaa aaaagattag acaaatggct aactagaata accagcatag agaagacctt |
| 13861 | aaatgacctg atggagctga aaaccatggc atgagaacta catgatgcat tcacaagttt |
| 13921 | cagtagccaa ttccatcaac tggaagaaag ggtatcagtg attgaagatg aaatgaatga |
| 13981 | aatgaatgaa atgaagcaag aagagaagtt tagagaaaaa agagtaaaaa taaatgaaca |
| 14041 | aagcctccaa gaaatatggg actatgtgaa aagaccaaat ctacatctga ttggtgtacc |
| 14101 | tgaaagtgat gggagaatgg aaccaagttg gaaaacactc tgcaggatat tatccaggag |
| 14161 | aaattcccca acctagcaag gcaggccaac attcaaattc cagaaataca gagaatgcca |
| 14221 | cgaagatact cctcaagaag agcaactcca agacacataa ttgtcagatt caccaaagtt |
| 14281 | gaaatgaagg aaaaaatgtt aagggcagcc agagagaaag gtcgggttac ccacaaaggg |
| 14341 | aagcccatca gacttacagc tgatctctca gcagaaactc tataagccag aagagagtgg |
| 14401 | gggccaatat tcaacattot taaaggaaag aattttcaac tcagaatttc atacccagcc |
| 14461 | aaactaagct tcacaggtga aggagaaata aaatccttta cagacaagca aatgctgaga |
| 14521 | ttttgtcacc accaggcctg ccttacaaga gctcctgaag gaagcactaa acatggaaag |
| 14581 | gaacaaccag taccagctac tgcaaaaaca tgccaaattg taaagaccat caaggctagg |
| 14641 | aagaaactgc atcaactaat gggcaaaata accagctaac atcataaaga caggatcaaa |
| 14701 | ttcccacata acaatattaa ccttatatgt aaatgggcta aatgttccaa ttaaaaaaca |
| 14761 | cagactagca aattggataa agagtcaaga cccatcagtg cgctgtattc aggagaccca |
| 14821 | tctcacatgc agagagacac ataggctcaa aataaaggga tggaggaaga tctaccaagc |
| 14881 | aaatagaaaa caaaaaaaag caggggttgc aatcctagtc tctgataaaa cagactttaa |
| 14941 | accaacaaag atcagaagag acaaagaagg ccattacaaa atggtaaagg gatcaattca |
| 15001 | acaagaagag ctaactatcc taaatatata tgcacccaat acaggagcac ccagattcat |
| 15061 | aaagcaagtc cttagagacc tactaagaga cttagacttc tacacaataa taatgggaga |
| 15121 | ctttaacacc ccactgtcaa cattagacag atcaacgaga cagaaaatta acaaggatat |
| 15181 | ccaggaattg aactcagctc tgcaacaagc ggacctaata gacatctaca gaactctcca |
| 15241 | ccccaaatca acagaacata cattcttctc agcaccacat cacagttatt ccaaaactga |
| 15301 | ccacatagtt ggaagtaaag cactcctcag caaatgtaat agaacagaaa ttataacaaa |
| 15361 | ttttctctca gaccacagtg caaagtagaa gttgggatta agaaactcat tcaaaaccac |
| 15421 | tcaactacat ggaaactgaa caacctgctc ctgaatgact actgggtaca taatgcgatg |
| 15481 | aaggcagaaa taaagatgtt ctttgaaacc aatgagaaca aagacacaac atactagaat |
| 15541 | ctctgtgaca catttaaagc agtgtgtaga gggaaattta tagcactaag tgcccacaag |
| 15601 | agaaagcagg aaagatctaa aattgacacc ctaacatcac aattaaacga actagaaaag |
| 15661 | caagagcaaa tacattcaaa agctagcaga aggcaagaaa taactaagat cagagcagaa |
| 15721 | ctgaaggaga tagagacaca aaaaaacctt caaaaaaatc aatgagtcca ggagctggtt |
| 15781 | ttttgaaaag atcaacaaaa ttgatagacc actagcaaga ctaataaaga agaaatgaga |
| 15841 | gaagaatcaa atagatgcaa taaaaaatga taaaggggat accaccacca atcccccaga |
| 15901 | gatacaaact accatcagag aataacacct ctacacaaat aaactagaaa atctagaaga |
| 15961 | aatggataaa ttcctggaca catgcaccct cccaagacta aatcaggaag aagttgaatc |
| 16021 | cctgaataga ccaataacag gttctgaaat taaggcaata attaagagcc taccaaccaa |
| 16081 | aaaaagtcca ggaccagaca gattcacagc tgaattctac cagaggtaca aagaggagtt |
| 16141 | ggtaacattc cttctgaaac tattccaatc aatagaaaaa gagggaatcc tccctaactc |
| 16201 | atttgatgag gccagcatca tcctgatacc aatgcctggc ggagacacaa caaaaaaaag |
| 16261 | agaattttag accaacatcc atgatgaaca tcgatgcaaa aatccttaat aaaatactgg |
| 16321 | caaaccaaat ccagcagcac atcaaaaagc ttgtccaccc taatcaagtg ggcttcatcc |
| 16381 | ctgggatgca aggctgcttc aacatatgca aatcaataaa cgtaatccat catataaaca |
| 16441 | gaaccaaaga caaaaaccac atgattatct caatagatgc agaaaaggcc tttgacaaaa |
| 16501 | ttcaacagga cttcatgcta aaaactctca ataaattcgg tattgatggg atgtatctca |
| 16561 | aaatagtaag agctatttat gacaaaccca cagccaatat catactgaat gggcaaaaac |
| 16621 | tggaagcatt ccctttgaac actggcacaa gacagggatg ccttctctca ccgctcctat |
| 16681 | tcaacatagt gttgaaagtt ctggccaggg caatcaggca ggagaaagaa acaaagggta |
| 16741 | ttcaattagg aaaagaggaa gtcaaattgt ccctgtttgc agatgacatg attatatatt |
| 16801 | tagaaaaccc catcatctca gcccaaaatc tccttaagct gataaacaac ttcagcaaag |
| 16861 | tctcaagata caaaaccaat gtgcaaaaat cacaagcatt cctatacacc aataacagac |
| 16921 | aaacagccaa atcatgagtg aacacccatt cacaattact tcaaagagaa taaaatacca |
| 16981 | aggaatccaa cttacaaggg atgtgaagga ccttttcagg gagaattaca aaccactgct |
| 17041 | caacaaaata aaagaggaca caaagcttat agataggaag aatcaatatc atgaaaatgg |
| 17101 | ccatactgcc caaggtaatt tatagattca atgccatccc catcaatcta ccaatgactt |
| 17161 | tcttcacaga attggaaaaa ctactttaaa gttcatatgg aaccaaaaaa gagcccgcat |
| 17221 | tgccaagaca atcctaagca aaaagaacaa agctggaggc atcatgctac ctgacttcaa |
| 17281 | actatactac aaggctacag taaccaaaat agcatggtac tggtacaaga acagagatat |
| 17341 | aggccaatgg aacagaatag agccctcaga aataatacca catgtctaca accatctgat |
| 17401 | ctttgacaaa cctgacaaaa acaagaaatg gggaaaggat tccctattta ataaatggtg |
| 17461 | ctgggaaaac tggctagcca tatgtagaaa gctgaaactg gatcccttcc ttacacttta |
| 17521 | tacaaaaatt aattcaagac ggattaaaga cttacatgtt agtcctaaaa ccataaaaac |
| 17581 | cctagaagaa aacctaagca ataccattca ggacataggc atgggcaagg acttcatgtc |
| 17641 | tgaaacacca aaagcaatgg caacaagagc caaaattgac aaatgggatc taattaaact |
| 17701 | aaagagcttc tgcacagcaa aagaaactac catcagagtg aacaggcaac ctacagaatg |
| 17761 | ggagaaaatt tttgctatct atccatctga caaagggcta atatccagaa tctacaaaga |
| 17821 | acttaaacaa atttacaaga aaaaatcaaa caaccccatc aaaaagtggg tgaagtatat |
| 17881 | gaacagacac ttctcaaaag aagacattta tgcagccaac agacacgtga aaaaatgctc |
| 17941 | atcatgactg gccatcagag aaatgcaaat caaaaccaca atgagatacc atctcacacc |
| 18001 | agttagaatg gcaatcatta aaaagtcagg aaacaacagg tgctggagag gatgtgaaga |
| 18061 | aataggaaca cttttacact gttggtggga ctgtaaagta gttcaaccat tgtggaagac |
| 18121 | agtgtggcaa ttcctcaaag gtctagaact agaaatacca tttgacccag ccatcccatt |
| 18181 | actggatata tatccaaagg attataaatc atgctgctat aaagacacat tcacatgtat |
| 18241 | gtttactgca gcactattca ggatagcaaa gacttggaac caacccaaat gtccaccaat |
| 18301 | gatagactgg attaagaaaa tgtggcacat acacaccatg gaatactatg cagccataaa |
| 18361 | aaaggatgag ttcatcatgt cctttgtagg gacatggatg aaactggaag ccatcattct |
| 18421 | gagcaaactg ttgcaaggac agaaaaccaa acaccgcatg ttctcactca taggtgggaa |
| 18481 | ttgaacaatg agaacacttg gacacaggat gggggaacat cacacactgg ggcctgttgt |
| 18541 | ggggtgaggg gagtgggagg gatagcatta ggagatatac ctaatgtaaa tgacgagtta |
| 18601 | atgggtgcag cacaccaaca tggcacatgt atacatatgt aacaaacctg cacattgtgc |
| 18661 | acatgtaccc tagaacttaa agtgtaataa aaaaatWaaa aaagtagata gtcccaaccg |
| 18721 | ctgtattctt tttgaatcct tttgccactg tttcatatca cagttttttt ttttttctga |
| 18781 | gccctagatt atcagtctgt tctttttttt tttttttttt tttaatgtag tccataggaa |
| 18841 | tctgctgcct cacRgtcagt atcgtgcccc ttaccccaat tctaatatga caggtttttg |
| 18901 | tctaagacag catttcctag aatgtgttgt aatgtacagc agcttcatgt gataattctc |
| 18961 | aaagactccc actgtcaaat gttagcatat tcaaggctct gccaggacct gtaatacata |
| 19021 | ttctttcttt attctgttta acttagcatt tctgaaacct atcttactac tgaactcttg |
| 19081 | acatttatta acaaacaaat acaaacRaaa ataccagggt tccaagtaaa attettatat |
| 19141 | aaagcaaagg gctttaaaca tcagagtttt ttagaatggt tattcactca tgcactcaac |
| 19201 | aaacacttat tacatagtat gtgccagcat aggggataca gtaataaaca aaacagacaa |
| 19261 | gttccctgtt catgtaaacc ttggacttat ggggcaaaaa gacaataaaY aagcagacaa |
| 19321 | aaaaaaatta caaacactct caatgaagga aaagagggta acatcgtaca ttgatggggg |
| 19381 | actataaaat taagtagtaa ggaaaagctg cacttgagaa ggtggtattt gggaagagac |
| 19441 | tcgaatgcct agaataagcc aaccagattt ggggcacagc actctcaaca aaggctgagt |
| 19501 | aacacagcca gtctgaggga ggacaggtct acggaatgag agccgaggca agtaaaggca |
| 19561 | acatcacgtg ggtcctgagg gtatgagcct tccagagggt gtgaggatac cattagaagg |
| 19621 | ttctgagcag ggaatgatag gatgtgattt acattttgta aagctgtctc tgtagaggat |
| 19681 | agtttgtaaa agtgcttgag gcaaaactga caagacttga agacttgcac aacagaactg |
| 19741 | aagaaatgag aagaatcaac tcctagactt ctgggatgat caatgtctag gatggacatt |
| 19801 | ggtgccattt gcaagatgga gaagaccggg ggaggtacgg atttaaagga gggtatgtaa |
| 19861 | ggagaaagaa gaatcctatg agacaagacc agaacatgta aatacatgaa gttttggatt |
| 19921 | tcttactctt caaagacatt ttacctcctg tccccgaagt tcttcacaga gttcaggata |
| 19981 | agaaaagctg ccagacagtt gagttttgaa taagtagtaa ctttcaaaca cttgaaagac |
| 20041 | attatattag aatagactac atccatcaaa ataNttaaaa atgtttttag aatagtccaa |
| 20101 | taatttggaa tatgaaagcc taagaaactt ccttatccaa ggccRcataa tataaccaaa |
| 20161 | attagaattt agtactccta gttggcaata tttttatgaa attactgttt tactaagaca |
| 20221 | ttttagaaat gaagacttta ctgaaggcaa taataagtgc aattaaaagt ggtacaataa |
| 20281 | Kaagacttct ttttcatttg caacaggtta gtagtgtatg agaattatga gatgtaaaat |
| 20341 | agtgtagagc ataggcttct ctagttggct tcatgtgagt tcgaatcttg gccctgcctc |
| 20401 | tcttaccagc aatgaaatat gcagcatttt acttaaggat actgggttgt atggagagaa |
| 20461 | tcagagttaa ggttggtgat ccagcagtgt catgtgtcag gagctaataa attgtattgt |
| 20521 | gaccagaaac actgaaactt gttataagga aagcaatcta attaaaacag ctgcattaaa |
| 20581 | atttctacaa tatcttccta acagcttatg ctatcaacag tttgtaaact atcagaatca |
| 20641 | cggatactgg ctgagaatgc ttttggagat ttcaaattgg cataattttc tcagaatgca |
| 20701 | ctgatctgac aaatgtacag acaggcagac aggaatacta ccactcttca tgaataactg |
| 20761 | tctgcctctc ctggatttct tccatgttgt gtaagtctat tacactttgg gtgaatgtag |
| 20821 | ctttagtaca ttgagttaat atcattcttg caaagaagct ctttataaat tccttaaact |
| 20881 | taatttgact ctggacacaa acaaatgaga acaaaataca gctcaaggat taagatcctt |
| 20941 | aaccaaatat gtaaaaaaca cgaaaagcct tacaaacagt gatcaacagt taaacatgcg |
| 21001 | ctatcacctt cccctaacag ctagggaagc atgtatatga atggaaeata tgaacttcca |
| 21061 | tttggtgtac taagaaaaca catcacgcag atggctttta tagcttccca aataaggttc |
| 21121 | taataattca caaaagtgtt gttccctaaa acccataata aacaataata aaacctaatg |
| 21181 | tttattgagg gtctactatg tgtcaaggag agtcctatgt actctcctgg aattacttca |
| 21241 | tttatttcta ataataacct ttaatgagca ctgtcactco cgtttcacag atgagcctta |
| 21301 | aggtcttagc tatcaacttt gcagacaaca cccaagtaaa atcctgcccc ctcttttttt |
| 21361 | tttttttttt aaccagaaat ccagtcctga caactgaaag cttatagggc atttccgctt |
| 21421 | agatacatag ccttacctaa attatatgtc taaaaactta actgtcatat tgtccagccc |
| 21481 | tcccccaata cccaactggc ttctcttcat gattttccaa ctataattct gtccattatt |
| 21541 | caggtttaga atttcatgct ttcttctggc tgtggtcctg ccaagtctta tgtatgctgt |
| 21601 | cacccttctc tgtgcaatct ttctcctact cattcctctc ttttcatcac tatcctattt |
| 21661 | caggccctta tcactcttac acctaattcc ctaacggttt cttcttgctt tcagtacaat |
| 21721 | ttccccttga tataatctat cctgcacact accaccagat ataaccttct aaagtcacct |
| 21781 | tattttagtt tcttcataat attttcctta tagtcaaatg taagggttct tcatgaatta |
| 21841 | taggacaatg tgcaaatggc ttgacctggc atttaagaca gtctgcagta tggcctcaat |
| 21901 | ctacatttct cacacctgcc actggtccta taatcacatc ctctgttcca gagaggctca |
| 21961 | ctcctccaga atgcccacta ccctcctctc cagcaaacat acaataaagc gaatccacat |
| 22021 | gaatacccca tcctaccctg ccttatccag cccctggaat accacctctt ccctattaga |
| 22081 | tgcctatata aatcctcagt attcaagatc cagcttaagg agcacttttt ctgggaccaa |
| 22141 | tcattctggt cctctgtatt gaatactcga actcctctac aggacaggaa cataatgtat |
| 22201 | tattacccta tcattcatta acacacaaca ggcttacatt atatggctgg ttgtattttc |
| 22261 | agaggtatgt ctatatgttt cgctatctat ctatctatct atctatctat ctatctatct |
| 22321 | atctatgtgt ttgaatacag atacataaaa atctcatttc acctataaga acctctggag |
| 22381 | cagatacatg tcatacaata attgggctaa aacaaaaatc cctcagtgaa aacctaaaga |
| 22441 | ctgggttgtg gttgtttgca gccaaaacgt acaaaatgaa aacctcctaa cgatttattt |
| 22501 | tccacatgaa attagtacca gatctagaga aaaatggaca aggatcaaat acaaataata |
| 22561 | aagtcatcac caaactaaaa atttcattca aaagtgatga tgttggccgg gtgtggtgac |
| 22621 | tcacacctgt aatcccagca ctttggaaag ctgaggtggg cagatcactt gaggccagga |
| 22681 | gtttgagacc agcctggcca acatggtgaa accccatctc tactaaaaac acaaaaaaca |
| 22741 | gctgggcgtg gtcatgcacc tgttataccc actactcagg aggctgaggc aggagaatca |
| 22801 | cttgaatcca ggaggtggag gtggcagtga gccgagattg tgctactgca ctccagcttg |
| 22861 | ggcaacagag caagactccg tctcagaaaa ttaaaaaaaa aaaagtgatg atgtcagtaa |
| 22921 | agacccaaat cagggtcata tgtttaacac cagaaagttt gtatttgcta caagacagaa |
| 22981 | accttgacaa aataactttt ctgtgctttg ttacttcatc tgaatgaaga aggtaaacca |
| 23041 | gttgatctca caaatctctt tctaagggct tctggaattt tttttttttt ttaaacaata |
| 23101 | aaaggaaaga gacttcYctt aagcagaaca aagtccgact cctctgtaaa gtggctgaca |
| 23161 | gcaagtggaa aatggaagaa gaacaattac ctgcaaaatg actcattccc ttgtaacaac |
| 23221 | tacactacta gggaattcct tctcttaccg gctgtggcta gcgtcagact catgacaaag |
| 23281 | acctcatcct ggggctagaa ctaggtttag ggttttctca ttcattcctc tgtttctcag |
| 23341 | ctgcattttc atttttaatc tgtgcaatga ttacagaggc ctatgtttgg ttttcatttc |
| 23401 | tagtttcccc aggaagagac aactactatc ttgatattga ttaacacttg aaacttctgt |
| 23461 | taatcacaat ttcagtattt cagggaatca gagaaatcat ttggaagagc cttgaaggtc |
| 23521 | atccagggtg agagaaaagc aaagcacaag caacagacca atgtttagac acagtgaagt |
| 23581 | gaagRcctac aggagaccct ctcaacttca ataagagtct tttttatggc tgtcttcata |
| 23641 | ttctttcaaa gaaatcataa taaaggagtt agtgagagtg taaataaaaa ataggctatc |
| 23701 | atagagtgct gggtaactga gctaaaacta aagtaaacaa actcagtggg ccatcacata |
| 23761 | tgcaagctgg taaggtctca taaatcacca tgactgggta ttgggatttc aaaggcacac |
| 23821 | tctgtaagct ttctcccagc cctgttgatt caagctccaa ttgcactggg ttacttatat |
| 23881 | ggcagcatat aaagttgacc ttttaYgttt aaaaatagtt ccattcctca tccctcctaa |
| 23941 | ggataggcca cagtacctat catatgtccc caccacacta cataatttat gtaagttttc |
| 24001 | acttaatttt caacacaatc ctttgaggta gattatgtta gtcctSattt acagggaaaa |
| 24061 | cagaggctca gggaggccaa atttatttat ttatccaagg ttgcaactct actgtagggc |
| 24121 | agatttatat ccagaagagg aattgcaaag ctcctcagaa tttaggagat ttaaaatgga |
| 24181 | atcaagataa aggttcacat ttttcatctt ctattccatt ttactcttcc agcatctaat |
| 24241 | aataacctat gatatattag aacaaatcag cccatactta ttttgacagt ctcttttcac |
| 24301 | cccatttcag tttgtacctt gctgtctcag agcccatcaa ccacctttcc tcactcccag |
| 24361 | gcagccggtc cttcagggga tggcttggca gttggcccac catcctctgc taccagggca |
| 24421 | tgactcatgg aggcaacagg tgaagctgaY gccaactatt agtgctaaaa aagactcaag |
| 24481 | cctcatgaaa tataccatac ataaatcgct ctccagtcag ctctacccct ccctccattt |
| 24541 | ctacagctga ctgtctgttc ttccttaaac atttacatcc gccactgacc tgtctgggat |
| 24601 | ttctttttta cttcaaggYt ctcctaaagg ttcagtMcaa ccaggtttca ccaaatgaac |
| 24661 | cctgcggtgg aaaggggcag caacgggcat tggtcagttt gcctccagcc aaggaaagtc |
| 24721 | tgactcccct ccttctccca ctcttctctt tctctgcctc acactgccta atacgtcttt |
| 24781 | cctctttctc tctttctttc cttatatctt cattttccac cctattcaga agtgaaactt |
| 24841 | cccttgccac tttgccccca ctaagaaaac agacctccaa cacaacctct taaYaggcta |
| 24901 | attttaaaat catgaacctg cttacacctt ccgtgagttt atcctatatc ttctctatct |
| 24961 | gctgtctgct tcccactaag gttttatccc tccatcccac ctccaaatcc tttacagctc |
| 25021 | agattctcaS atctgcttta gtcacttggt ctcctcatcc cactgaccct attatccttc |
| 25081 | tgtgcacatg ttagagggca catgctcctc ctaggaacta gggccaggtc agttatgctc |
| 25141 | ccatcctgac atgacctaga gaacaaaaaa ggcaaaacga ggtactgtag gattcctctc |
| 25201 | taggaattat actttgctga aattgctctt gctattttgg aatctagtgc tcactaaata |
| 25261 | catttttaaa atattatttg gatatatttt gaaaatacaa ccagaggtcg ggcacagtgg |
| 25321 | cttacgcctg taatcccaac actttggcgg gccaaggtgg gcagattacg aggtcaggag |
| 25381 | ttcgagacca gcctggccaa catagtgaaa ccctgtctct actaaaaata caaaaaatta |
| 25441 | gccaggcgtg gtggcaggcg cctgtaatcc cagctgcttg ggaagctgag gcaggagaat |
| 25501 | tgcttgaacc tgggaggaga aggttgcagt gagcctagat cacgccattg cactccagtc |
| 25561 | caggcaacag tgcaagactc taactcaagg aaaaaaaaaa aagagagaga gagagaaaat |
| 25621 | acaaccagag aataaacact tgttcttcta tctctccatc ttccccacca atacaacatc |
| 25681 | cagaaaatag tcatctttac acttatttag aagcacatag atcttataaa aStttaactg |
| 25741 | agcattaggt tttcatcttt aaaaatattc tgaggctggg tgcactggct catgcacttt |
| 25801 | gggaggccaa ggcaggagga tcacttgagc ccaggagttt gagaccagcc tgggtaacat |
| 25861 | agtgagaccc tgtctctaca aaaaaaaaaa aaaaaagtta aattaaatta aaaaacagaa |
| 25921 | aagccggtct ggtggcacat gcctgtggct ccatccactc aggaggctga ggtgggagga |
| 25981 | tggcttgagc cagggagatc aaggctgcag taagccatga tggcgccact gcactccagc |
| 26041 | ctggttgaca aggtgagaac ttgtctccaa aaacaaaaca aaacaaaaaa cgttttgaca |
| 26101 | tgtagaatac tgtttctggg actaaattag agatttaagg tttgaggtat gagaaaatac |
| 26161 | ctggaatcat gccttacatt agaaggtaag gtaaatactg gtgtttccca atgagttgta |
| 26221 | gtcagattgt tcatatgtat caaacagaca atatactgaa atatttaaaa ataaggctaa |
| 26281 | atagcaacaa ataatttttc ccatgaattc tacttttttt tttttttttt ttgagatgga |
| 26341 | gtctcactca gtcgcccaga ctggagtgca gtggcgccat ctccattcac tgcaacctct |
| 26401 | acctcctggg ttcaagtgat tctcctgctc agcctcccga gtagctgaga taacagacat |
| 26461 | gcaccaccac acctggctaa tttttgtatt tttagtacag gccaggtttt gccatgttgg |
| 26521 | ccaggctggt ctcaaacttc tggcctcatg tgatcgacct cccaaagtgc tgggattaca |
| 26581 | ggtgtgagcc accatgccca gctgaattct actttttttt tttttttttt gagacagagt |
| 26641 | cttgctctgt cacccaggct ggagtgcagt ggcacgatct tggctcactg caacctctgc |
| 26701 | ctcctgggtt caagtgattc tcctgctcag cctcccgagt agctgagatt acagacatgc |
| 26761 | accatcacac ctggctaatt tttgtatttt taggagagac gggatttcac catgttggcc |
| 26821 | agctagtctc taactcctga ccgcgtgatc tgctctcctc tgcctcccaa agtgctggga |
| 26881 | ttacaggctt gaggcaccac gcccggctgc tgaaattcta cttttgaaag aacaaattcc |
| 26941 | ccttctgtag ggactgatga ttcagtctgt aatgatgcat tctcttcatc ttcttttttt |
| 27001 | gtcttttctt ctatatatcc tttaggcatt ctactgtatt aacactagat agaaaaatta |
| 27061 | agatttaggc ccaaacatta tcttctggca taaagtaaac taatcKtttc ctgatgtaca |
| 27121 | ataaataaac agacatattt ctagtagaag attccttatg atttatcaaa acccaattat |
| 27181 | cacttaccct tatggagatc ttagatctta gaaaccatac tgacaatata agattaagaa |
| 27241 | aacaacagaa aatcaaaata tataccaagg ataatgttaa taggtttcat ccttatcaca |
| 27301 | gcatactgtt gaaacttact tgaaatattt ttttaaaaaa gatacaagag gacaagtgtt |
| 27361 | tagtttttaa caagggaggg gactagtcaa caaaYattga ttacctaatt tatgaacagc |
| 27421 | tctggccaaa gtggtcaaaa aaaaaaaatt aggactttag ttttcctaaa cctatcttaa |
| 27481 | tttttaacag gattatagct ttgttcaatc tttacagtct ttagcctctt ttgtgaaatg |
| 27541 | ggtttccttg taaataataa aatgacacct atatatattt aaagcactca tgaaataatt |
| 27601 | ttatctcttt attaaaaact gtgataaccc ttccaatttt gaaatttatt gtcttcctaa |
| 27661 | attcttgcta caaagcctat cattaatatt gcagttcatt ttttccctaa agattgaaaa |
| 27721 | aaacactaac aaatataaag tctttatggt caaagaatgt actcctttta taaaacttgg |
| 27781 | agttgatgta aaggcacaga caacacaagt taccttaata ataaatgcac ttttctttgt |
| 27841 | aattttgggt aattcagtgt gatttgtaat tcttttaaaa gacacacaat cacttgtatt |
| 27901 | tttgcatggt tctttttttc ctaatttccc actatagtga aatatctgtt tttccttctc |
| 27961 | tattgggttt ggattggaat gatcaggtag aattaaatgt aaaataacaa aaggaaaata |
| 28021 | ctttatagat cttctcaaaa tactttgaag ggtataatct ctttaagatg gctttattca |
| 28081 | tatttaagcc aaaaacaaaa agcaaccaaa acactactgt ttatacaaaa agcagcttca |
| 28141 | aaatcagcca gttattttct gaaatggaga tggcagacat tagcctgcac ttactccaat |
| 28201 | atggcagccg catctttgcc tctttgctat ggacacatcc ccagcaggcc tgtacttect |
| 28261 | actctccaga cctgtttatg acaggccaag gtgtttccta ttatggtgcc tatggctgca |
| 28321 | cagtgcactg cttgtcagta ataaaagcca gaggctgtcc ctatagggat tacagtgcag |
| 26381 | cagaccagag atgctgatgg gaatgcaagc atgccctagg caaacattgc agaatactct |
| 28441 | ctgacaggca aaagccaaca aacacatcct ttcttcccag actagcaggg aaagtattca |
| 28501 | gttatgtcat tttcacttgg aacaaactaa taagaagaat gttattgcta aaataggtag |
| 28561 | agatgagaaa gagcagttta tgaccatgaa tatggcctct atggtttttg cctgtgcact |
| 28621 | acaaactttt aatttaaacg agatgagatt gatctagcat atactatgat aaaatttctg |
| 28681 | agacatgagt atctaaaagc agaactgaga ttaagtgaga aatgctcagg attcctgtca |
| 28741 | ctgaaagaaa aagaaaaaaa cctttcttga aaggcacaaa gtagaatgct cagactctcc |
| 28801 | atctaccaat acaaggtaaa aaacagcatt tgcattaacc aacatcatgc attcttgtac |
| 28861 | taatttatgt gactaactac aaaatgttat attgacactc actgtgtagg gtgtcaggat |
| 28921 | ggccaagtgg tctaaggcac cagacactca ctgggttggg ctaaaaacac Ygtagaagac |
| 28981 | ctcatcagaa caataaaaca gagacacaaa atattgtact aagagttaag agaattacaa |
| 29041 | ataaaagttc tgtggaaagt caaaggtatc caattaggaa agcctgaggt gggagacata |
| 29101 | ggacacccca gatataggat gggaagaaga tgggattaaa aaaaaaggct ttataaagga |
| 29161 | tgtaacattc cagataggcc ttaaaagatg gcttcgatct tttaaataaa actttttgtt |
| 29221 | ttagaacaga tctagattta cagaaaactt gtgtagacat tacagataat tcctatatac |
| 29281 | tctacactgt ttcccatttt taatgtctga ctttagtatg gcacatttgt cacaattatt |
| 29341 | gaaccaatac tgatacatta ttattaaaca aagtccacac ttcattcaga tttccttagt |
| 29401 | tttcacgtaa tttccttttt tctgttctgg atcccatcca ggataccaca taatatttag |
| 29461 | tcctcatgtc tcctgaggct cttcttggct gtaggtatct caggttttcc ttgtttttga |
| 29521 | taaccttgac agttttgagg agtactggtc aaatactggt caagtttcga gggtagaata |
| 29581 | tccctcaact gagatctgtc tggtatttgc ctcatgatta cactggggtt atatgttttc |
| 29641 | agtagggaaa ccacagatta aaagtgccat tttcatcaga tcatatcgag ggtacatgtt |
| 29701 | atcacttatc actgtcaatg ttgaccttga taacctggct gaagtagtga taggWttctc |
| 29761 | cactacagtt tctttttttt ccctttctac actgtactgt ttagaaggaa gtcaatatgc |
| 29821 | acagctcaca cttacggtgt agggggttat gttctccctc cttgagagta gagtatctat |
| 29881 | agaagttatt gggaattccg cacaggagga gatttgtcta ttttccccca cttaggtatt |
| 29941 | tatttaataa attgtatcaa tatgggttta tggatattta ttttaccctt tgggatataa |
| 30001 | tccaatacta tattattttc ttgctcaaat tgttctagct ttggccattg ggagctcttt |
| 30061 | cagtttgctc ttgcaactct tcagtatatt cccacgggtg tagggttcca tttgtttgag |
| 30121 | caggtcctta ctttttggca ctacaagatg ctccaggctc atcttacata tttccttcct |
| 30181 | gagtccctag aaatcagcta ttttttttca aagagtcttg gttcctttta ccagagaatg |
| 30241 | gtattagaaa ccaaaatgag ggtgctaagt gtgctcattg ctactggggt atggtagctt |
| 30301 | ctagaccctc tcggctgata gagcaaggaa atatttgtgt gtatactaga tggtaagatg |
| 30361 | ttgagaggta gaatggagag ttaccttctt aatatacttt tcttaatatt aaatattaat |
| 30421 | aagatattgt gtgcctgggt tactttcagt cttatgcaga ggaagaacgt atgtacattc |
| 30481 | atttatgaaa aacactggct agactattct tctgcactgc attatgcatt tctgctagaa |
| 30541 | aaagctcaga gattttcaga tattctgaca aaaatctcag gcttgcttga tgagctatac |
| 30601 | tgtaaaatta cagaaggaaa caaagggcaa actccttgaa ggatgaaaat aaaggaataa |
| 30661 | aacatataaa acaccaccaa aaaaagtgat taatggggga gaagagtaaa taaaacatta |
| 30721 | taatagtcct ctttggttag tatcaatatt attaggtcac tttatcaaaa ggatagaaat |
| 30781 | caggatggca gacacccttc tctaaccctc accccccaaa ataataaaat aaaaactaat |
| 30841 | gttattcctt cacatatgga aatgaatgag ttctactgag tatctagtat gattcctgac |
| 30901 | tcagtcgaga caattcccat cttttaattc attaaatcac cacagcaact atttaacaga |
| 30961 | tgagaaaact gaagcacaga aggcaacRtg gcccaattta cgtttggtaa aaggcagaga |
| 31021 | atgaatccaa ttcaggcagg ctgtctatag tagggtgctc taaatccacg gaccattctg |
| 31081 | cctaaggaat gaatatttta tgaattggaa tatttctgct gaacgaattt gccattttct |
| 31141 | cctctagtgg agaaatccaa ctgagggtaa agaaggaaaa atgttgattg tttaaaacaa |
| 31201 | taaattagtc tctaaattgt tcttgcagct gatttagact agtgtgtcca aaagtatgcc |
| 31261 | cagtgttagt aaatggtagg caaaatgctt taaggtggtg caagatgaat gttttctcat |
| 31321 | tttgttattc attaatttta gaaaaaatac agaaaaaata taacttgaac atcaaaccta |
| 31381 | tgatttcatg tataccatta cttaagatga ggcacttggg tgagtattta gatttaacaa |
| 31441 | taagttgctc ttagcatttc tatactttga caataaatta tccaaaaaag aaattaagaa |
| 31501 | aataatccca tttgcaatag catcaaaaaa atacttagga gcagccaggc gtggtggctc |
| 31561 | actcctgtaa tcccagcact ttgggaggcc gaggcgggca gatcatgagg tcaggagatc |
| 31621 | gagaccatcc tggctaacac agtgaaaccc catctctact taaaaataca aaaaaaaaaa |
| 31681 | aaggcaggtg tggtggcagg cgcctgtagt cccagctaat tgggaggctg aggcaggaga |
| 31741 | acggcgtgaa cctgggaggt ggagcttgca gtaagccgag atcgcgccac tgcactctag |
| 31801 | cctgggcaac agagcgagac tccatcMcaa aaaaaaaaaa aaNaaaaaac ttaggagcaa |
| 31861 | atctaaccaa caaggtgaaa gatctataca ctggaaacta caagacattg atttaaacaa |
| 31921 | ctgaagaaga cagaaataaa tgaaaagata tcccatgttc atgcactgaa agaattagta |
| 31981 | atgttaaaat gtccacacta tccaaagcca tctatagatt caatgcagtt cctctattga |
| 32041 | ttcaatgcaa ttcctttcaa aattccaatg tctttttttt tcacagaaat agagaaaaaa |
| 32101 | atcttaaaat ttatatgaac caaaaaaaga cctaatagcc aaagcaatct tgaacaaaaa |
| 32161 | gaacaaagct gcaggcatca tattccctca tttcaaaata tattataaag ctactgtaat |
| 32221 | caaaacagca tgttattgac ataaaaacaa acatcaacca atggaacagg atagaaagcc |
| 32281 | cagaagtaaa tccaagtatt tacagtcaat tgatttttga caaagggtcc aagaacacac |
| 32341 | aatggagaga ggacagtctc ttcaaatagt gttgggaaaa ctggatgtcc acctgcagaa |
| 32401 | gaaagaaact gtactcttat ctcacactcc atataaaaat caactcaaaa gagatataaa |
| 32461 | ggcttaaaca taagacctga aactgtaaag ctactagaag aaaatataag ggaaaatctc |
| 32521 | cacaacaatg gtctgggcaa tgatttcttg gataggactc caaaagcaca ggcaactaaa |
| 32581 | acaaaaatag acaaatagga ctacattaaa ctaaaaagta cctgcacaag caaccaaata |
| 32641 | atagagtgaa aagacaaccc atggatcagg agaaaatatc tgcaaaccat acatctgata |
| 32701 | agaggctaat attcaaaata tataaaaaat tcaaacaacc aaatagtaag aaaacaaatc |
| 32761 | gattaaaatt agRcaaagga tctgaaYaga cacactttcc tcaaaaaaga aatatgaatg |
| 32821 | gccaacagag atacaaaaaa aaaaaaaaaa aaaaaaaaca atgttcccca gccctgcaca |
| 32881 | gtggctcatg cctgtaatcc cagtgctttg ggaggctgag gcaggagatc acttgaggcc |
| 32941 | aggaattcta gacaagcctg ggcaaaatag caagaccctc tctatacgaa aaaataaaaa |
| 33001 | gccaggtatg gtggcacaca cctgtagtcc tggctacttg ggaggctgag gcaggatcat |
| 33061 | tgagcccgag aatttgaggt tacagtgaga catgatcaYg ccactgcact ccagcctgaa |
| 33121 | caaaagagca agaccctgtc tcaaaaacaa acgaaaactc aacaatatct ctaattctca |
| 33181 | gggaaatgca attaaaacca caatgagcta tcacctcatg cctgttagaa tgactatttt |
| 33241 | cctttttttt ctcttcaact tttaagttca ggggtacatg tgcaggatgt gcaggatgtg |
| 33301 | caggtttgtt acataggtaa acatgtgcca tgtttgcttc acgtatcatc ccatcaccta |
| 33361 | ggcactaagc ccagcatcca ctagccattc ttcctgatgc tctccctgca cccatcccac |
| 33421 | ccgccaacag accccagtat gtgttgctct ccaccatgta caatggctat gttcaaacag |
| 33481 | atgaatgata acaagtgttg gcaaggatgt ggagaaaagg gagcccttct acactgttgg |
| 33541 | tgggaatgta aattagaacc accattattg aaaatagtat gaaggttcct caaaaatcta |
| 33601 | aaaatagaac taccataaaa tccaacaatc ctacttctag gtatacaccc aaaggaactg |
| 33661 | aaattggtac atcaaaaaga tatttgcact cccatattca tttcagcttt atttacaaca |
| 33721 | gccaagatat ggaagcaatc taagtgttca tcaagtgtgc ataaagaaaa tgtgatatat |
| 33781 | atatatatat atatatatat atattatata tatatatata tatatataga ttatacaggc |
| 33841 | cgggcgcagt ggctcagatc tgtaatccca gcactttggg aggccgaggc gggccgatca |
| 33901 | caaggtcagg agatcgagac catcctggct aacatagtaa aaccccatct ctacttaaaa |
| 33961 | atacaaaaaa ttagtggggc gtggtagcgg gcgcctgtag tcccagctac tcaggaggct |
| 34021 | gaggcaggag aatggcatga acctgggagg cggagcttgc agtgagccga gatcacacca |
| 34081 | ctgcactcca gcctgggcta caaagcgaga ctccacctca aaaaaaaaaa aaaaaaaaaa |
| 34141 | aaaaaaagat tacacagcct tataaaagaa agaaattctg tcattcacaa catcatggaa |
| 34201 | tggaactaca ggacattatg ctaagtgaga taagtcaggc acagaaagac aaatattgtc |
| 34261 | tttaataaat gatctcactt tatggggaat ctagaaaagt cgatctcaga aacagagagt |
| 34321 | agaaaggtgg gttaccagag gctgtagagg aggagggaga gagaaatgga agatgtcgat |
| 34381 | caaacagtac aaaatttcag ttagattgga ggaataagtt ttagtgatct attgcactgc |
| 34441 | atggagaccc cagtaattaa taacgtatta tatgttttac aatttctaaa atagattttt |
| 34501 | gacattctca tcacaaaaag ataaattggt gaggtaatat agatatgtta attagcttta |
| 34561 | ctaaatcttt ctatgatgta cacattttaa aaatcacatt atacccaaga aatagataca |
| 34621 | attaatattt gtcaattaag aataattgac aaataaattt taaagattaa taaaaaatac |
| 34681 | attaagttaa aacattcaac atataatagc agatatgaat gtacacagaa atgacaaaat |
| 34741 | cccagcactg tgggaggccg aggagggagg atcacgaggt caagagatcg agaccatcct |
| 34801 | ggccaacatg gtgaaacccc atctctactc aaaatacaaa aattagctgg gcatggtggc |
| 34861 | atgcgcctgt agtccagcta ctcgggaggc tgaggcagga gaattgcttg aacttgggag |
| 34921 | gcggaggttg cagtgagctg agatcgtatc actgtactcc agcctggcga cagagcgaga |
| 34981 | ctctgtctca aaaatgacaa agtttgagca acattaattt ggactgtaaa ttcttaaaga |
| 35041 | atagaaaagt catgtttttc ttctaagaaa tattatgttt ttacattgca agaaatatgt |
| 35101 | gacacagagt ttttcttaat ctctcttttt tagtatattc tagacaggca agcacatgca |
| 35161 | atttattgag cacccagtgt gtgctaatgc accaaacgga gctttacata cattatagct |
| 35221 | aatggtctca ataacttttt cattttattt tcccctttca ttatcgccac ttataaatga |
| 35281 | aaaaactgag ctccagagcg gtaaggccta ttgcccaaaa tattagatag gaaacaacag |
| 35341 | agctggaatt tgaaaccaag tttgcctaat gaaaaacatt tatgttcttt tcactgtaca |
| 35401 | aagctgctgt aagtatcact ggtgcctact ccaggccagg gagtaaatat cattcatcct |
| 35461 | tatcacatac aaagtataac aaaacaaaaa caagaagctc tgaatactaa ataataataa |
| 35521 | taaatacttt ataaaagata aactattctc taagagtaaa agataataaa cctattgtca |
| 35581 | ttgtagttgg tcactgaact taactaattt cctaatggac gcagcagaat tgtttgttgt |
| 35641 | ttaagcaaat atcaaggcat accccccaat ccctgttaca gggaataaaa aaagtcttct |
| 35701 | tcaaatgcct ttcaaaacta atgtgaatat taaaataaag tgctactgat ctctctacat |
| 35761 | tctagaaaga tctcagacta acagtgtcaa ttaaactatt acaacaaata gccatgtaaa |
| 35821 | aactgctccc ctactagagc tacgctatca caaaattaac cccagcaatc tacagttcta |
| 35881 | gaaaaagagt tggtccctcc tgctgaaaaa gagaaaacca aaccctacat attttgtgaa |
| 35941 | tccactgaaa ataatgacat aaacagcact gcacatccat catgtattta tatcttaagt |
| 36001 | gatagctaca gtctatgtaa ctatcgcaga aatcttctta aactgaacaa aagcattatc |
| 36061 | tatcatcctt tctctctaga aaaataccag tttgtttaat ctacccataa cattagaaaa |
| 36121 | aatacttctt ttatcatggg caatccttat gtttcggaat ttttttcgaa aacgtagaca |
| 36181 | gttcataatg ctctcagaca gaagttacca ctactacccc aaatattaag tcaaaatgta |
| 36241 | actgaaaaaa agaaatttat ctagtaatga ttaatttatt gaataattaa aaattaggag |
| 36301 | tttttgataa ttaaatgttg ggaataatta aactttcact aaatactggc acaggaactg |
| 36361 | aacggatgac aacagagaca agttaagatt tgttttaatc ctaagtttta tgctaaggta |
| 36421 | acatcaaggg ctctactttt cagtgaaaga aaaatttcct ttaaaattgt ttccctcttc |
| 36481 | cattattact taaaagcaaa gaaagtattt tttctaacag tgattatagg attctcaatc |
| 36541 | tttcatatta actttaaggt actacatgta agctacataa atctttccca aatctacctt |
| 36601 | tattgagtag atgaaaaagc agatatcgaa ctcaaaacat ctaaatgttg attttctata |
| 36661 | attatagaag atattttctt ttgaaattta aagtttgtat ttaggcatat taacactttt |
| 36721 | ttaatctaag atttaatatg ttaatgtaga aaacaactga ggctgttttc tctctataaa |
| 36781 | actaacatgc cttttccaat taacttagtt tccttcttag caaaagttta acatttatat |
| 36841 | ctgttgagtt acaYtgtagt tgcataatgt ggaaatatct taagatttag ccacttctat |
| 36901 | gaagatccta tatttgttat aacacccctc ttgcccagaa gaaaatacat caacaaaaag |
| 36961 | tcctactata tgttctgcat tgactattac aaagcagtag ggggagcagc cagagaaagc |
| 37021 | tctgagacaa agcacccagc aaagcaattt aatttatagg caacagaaac atgctagaga |
| 37081 | ggtttgccaa aagccaaata cacaaaataa attccataaa gaagtcatga ccacatttgt |
| 37141 | aaagctggcc taagtatatt tggaatgaag tatagaaaaa tctgaagtct cttagaaagg |
| 37201 | gtctggattc taacttcaag acagactttc agcttgaaac acagtcattt ccagccatgc |
| 37261 | caaacctcaa actgccccca aaattagagc agaaacaaaa tacattttta tagtgatatg |
| 37321 | tcttgggaat aactcaaaat gatttcgttt tctcctttga acctcactga acttacaggg |
| 37381 | aaggcctggt gaaatgtgca ttattaggtc acaagaggat aacgttacag ggttccatag |
| 37441 | cataatcctt gaaagccctg aggcccaggc cgctgtcctg ctctcctctc tactgactcc |
| 37501 | catcctcaat atactttcca tgagagaaag gtaaccaaaa acatggtgaa actattctgc |
| 37561 | ttaaaaggtg ttttactgaa aaggcacatt gaccactaac ccttattaca tttcacatgt |
| 37621 | tctaaaaatt agtaaggtct gcattttcag ttttagagta aaacaggaaa agtacttagt |
| 37681 | tagctcttct gtctggcatt ctgtaactgg aatttaagaa aagcctaaac ttctaattag |
| 37741 | aacttcagaa tgttcatgtc tgtattatac cacaaaaact gtaaatataa tttaagcatt |
| 37801 | tttatgatac agaaatgtgc gctatagaca gttatctaag aattataaaa atccatcaaa |
| 37861 | cttgaagtgt ctggtataaa ttacaaccga atggtaaaaa gacattttag ccacgtaggc |
| 37921 | aaccaatgaa gtttgaccta aaacccaaat aatagcaata acaacagata cagtgcctac |
| 37981 | tattgtcaga tactatatta gctagctctt ttgtatatat tctttKgaac tcttacactg |
| 38041 | acttttcaag atagttatta tggtgcccat tgtatgctaa ggaaactgaa gctaaaaaaa |
| 38101 | attgaagtca tttctctaat gtaactccat tagcaagaaa caaaaccata attcaacttc |
| 38161 | caggtttgcc tgcctccaaa gctttgagac ctcactaaaa ttaccctcaa ttgtagcaat |
| 38221 | gaggttccaa aggaaatctg tacaaatctc aggcagatta atgtgcttat aagggtaaat |
| 38281 | gccatagatt taatcactca aattaaatgc catctggaat ctcttaccct acttggtaca |
| 38341 | agcatcctac atgtgttaca gccaacccta gccttggcca agaattcaat aattttYctg |
| 38401 | cttcaatgcc atcaacaagc aagaaacata tataatacca atacaggaaa tacagctagt |
| 38461 | tgctcctaga gcatcattta aaagctgcta attggctaag atgcctttta ttgagaagtc |
| 38521 | taagaaacag aaataatgtg ttaacccagt ttgttaaatg ggtttgagga atcagtttcc |
| 38581 | tccttcctaa tacctaaatc catctccata ataagtattc tctatcacca tttaaaaatc |
| 38641 | ctgatctctt actagtgttt ttattgtgct tttcaatctR ttagagtaaa ttttttaaaa |
| 38701 | cctcaagaga gaacacagaa aactgtcata atttttcaaa ggctaaagga gaataaactg |
| 38761 | aaaatatcag tttgaacacg tgtatgtaaa aagaatggat ggtaataaaa ctaacaactg |
| 38821 | aaaagcaatg tttctgactc attcattcat actcaagaaa tatcaaataa ctgagtggag |
| 38881 | atgatgcaag ctgtacacat ttccagttca gactgaaggt gtggcaacac tcacaggggc |
| 38941 | cttccgatca gctgctccaa ctcagtttac agcttttgtg gatgtggctt attcatgccc |
| 39001 | ttgttatgaa gaagaagggt aactttacaa cctttagcaa gagctttttt ttctttctgt |
| 39061 | gctaaccctt ttttgtctat aacagtcatg taagcttagc actgatttag aaataaataa |
| 39121 | gttaaagaag acacgaatgg cttggacatt aaaagtttaa gcaacagctt aagctgagct |
| 39181 | aagtttaatg ttaaaaatta gtgtgacagc aataattttt caattcagct ctgtcgtggg |
| 39241 | ctaggaaaaa atatcctaat ttttcagcca caagacataa acattgctag aatgactaca |
| 39301 | ttttgaaaaa attatcttct cactttacct agatttcatt ttaaccaaac agaccaacct |
| 39361 | ctcctattaa acctgtaaat taactcaaag tctaatctgg ggccttcata ataaagattt |
| 39421 | tttttttaaa tccctgtctt atctggtgcc tttaaaagaa aggaatcatt ttaggcaatt |
| 39481 | attatgacct aaaaaatttt taaatcttaa cgtatagatt aattccttga cagctgaagt |
| 39541 | catttacaat acattattaa atttcacatt cagttttaaa tccaaacata gatctcaaca |
| 39601 | ttaagaatat ggattttagg gaaagYaact agctgctcaa attcatttag ctgaaaaact |
| 39661 | actgtcgaat caccttgaaa aYatacacta atgcttgcac attgctaagK ggcttttgat |
| 39721 | tctgctcttc attttcttac acagMatcaa ccacacaata gaattaagaa ctcaggccca |
| 39781 | tggcaaagtg ctcccattta gcagttagga cactggacat attcagcata tgtctactgc |
| 39841 | tattgagaat caaattatga aaacaaacta tattttataa aactaagaat ttgttactca |
| 39901 | Ratgagaata aaggtgccta atgcYacagc agggggtaaa agtcccagta agagagcaat |
| 39961 | aattggtcat tctgatgagg agctgtgcca aatttagaaa tgaggcacaa atgacaaaaa |
| 40021 | aattgatcat cagtgtggac atgggtaggt cagtgtgcat atgggtaggt tagtatctgc |
| 40081 | tggccgtgtc tacagagccc cttttattta tttgaagcca acttttcact tgacaatgct |
| 40141 | acctacaaaa gcactgttaa tagataagac aatcaagtgg ctgttatgcc gtggcctttt |
| 40201 | tgtaaaaact gcatgagaac aggtgggata aaaaccatag aaaggtagaa aaaaaaaagc |
| 40261 | atcactcatt taaagatatg tgcacataga tgaactgtaa gataatataa ccttcttgac |
| 40321 | cttgtaaata ggtggccaaa aatacttaaa tatctKgacg aggcaactat tactaattgt |
| 40381 | ctcacaacat taYcagtcct tactggctga atccttcagc catctgcata cgacatgcac |
| 40441 | aggcaaatag ccactaaaaa catacacaat tccaattatc tccttgcata actaaagaca |
| 40501 | aaaagaacag gaaatccaaa aaatacagca aaactgaaaa cactatacct tgccttccgt |
| 40561 | gctattttaa ggctgtccca ggctgaaggt tacttctcag ccctcaaaac tccattaact |
| 40621 | gccaagacca gcaccttagc acatatggca cagatgccta gttgaagcct ccttgtacta |
| 40681 | aatgacctca aaaaactggg ctttctttcc aagattaatg gaaacaaaag taatgatcat |
| 40741 | aaggaacctt ctaactctgt taatgtttcc tgcggatatg tatggccaac tgaatatata |
| 40801 | aaggctcagt tcttgcgggg taccctgaca actagatgaa ttagggtgaa ggttccaagt |
| 40861 | tcgatgaact cttctgagtt tgagtgccct aggtggtgac attcaagtga tttcacagac |
| 40921 | atccaccaat tcttattttc aaccgggaat cccaagaaca cctttaaaca tgcaatgccc |
| 40981 | atggacccta ctgggtttct gctgcaaggg ctcagtgtta cttaggaacc ctgtgtcttg |
| 41041 | ggagatactt aaatttagca cgtcaaattg cttttggaaa atgtttatct ggcgtgacta |
| 41101 | ggggaggcaa agcaaggaaa caggaacctc cctctaagga gcagaatagc caacatgcag |
| 41161 | taagcctaaa aaacctctca atttatttcc cggggctatg ggtaccagtg gctcgccggc |
| 41221 | atgggaatcR gcagcctggg ggcaccagtg acagcagaac cggggcttac cttgttggag |
| 41281 | gccatctcac tgacggagtg cctggtctgt agggtctcta gctggtccag acaccagtcc |
| 41341 | agctcctcca gggtctcgct ggccagtttc tggtaggcct cctctgcgaa gagacaggga |
| 41401 | aagggggact cagttctcaa gcgcttcacg ggtccgctag cgagttcaaa gggggccatc |
| 41461 | ctgccatacc ccgccatgct cggatgcccg gtgccggcac atgagggctg ctccttcata |
| 41521 | ttgcccagcc cggcagtgca acgggggcgg aggctgtgct cgcggggcgg ccggcctcgg |
| 41581 | ggaggcacgg tctctccagc ttggcgtctc gggtcggcct ccagggaggg cgtgcaaaga |
| 41641 | gcggcgagcc aactttcaca cagcgacgcg agcggaaaaa ccactatgcg acttttcaac |
| 41701 | tgtgcggcgg gcccgagtcc cagtccagga gcYcggctct agcggatggc gagggggtgg |
| 41761 | cgagcgcgct tgggtgcccg ccccgcagag gagcgtctgg tccacagccg gatctcctcg |
| 41821 | gtccccaacc cggagccgcg gccccacgcc cgccccgcct gccgagcctt ctgcacKgca |
| 41881 | cttgaagtga atgaatcagc cgcggccggg tgcgcggcca ccacgtttcc tgttttcttt |
| 41941 | ctcaactcct cgggcggcag gcgcagcgcg gctccaacgc cgcagctttc cgggaacact |
| 42001 | ccccctcacg cccctctcgg ccctcggcgc tgcctggctc ctcctcaacc cccttacgaa |
| 42061 | aagattccga attgcaaaag cctgtccctg tccgtgccac ccgccgcgtc cttcccctcg |
| 42121 | agctctgccc cgggggcccc gccacctcgc agtgccatcc ccccggcgca gccgggcgcc |
| 42181 | actcgctttg aagtgcagac gaggaggcgc gggcgcgagc gtgtgtgtct gtggggctgc |
| 42241 | tgtgggactg tctgtgctag gagagcggcg gaaaacccgg cgtggagcgc ctgcagtccg |
| 42301 | ccgtccgggg tcgcgcccgc cgcggggctc aggagggacc ggcacgcagg cgcgcacggc |
| 42361 | cgcgcacctg tctcgctaga accccgcgcg ccccgccgag ccactgccct ccaggttgcc |
| 42421 | cagaccgctg gagcctctgc gactccacga ccggctgctt cctgcgaagt ttcacaaaca |
| 42481 | aggttattta ggaccctcaa ggtttctgag ggtttaagtt ttagggtttc tgggggttcg |
| 42541 | tttctaaagg cttaggattc acctacttca aggaggcagg gttgtgggaa ttaagaccgc |
| 42601 | attctttttc tacccagccc agtccctact aagtaccaga cttctaaatg ttggggcgga |
| 42661 | cagaaacaag caaacaaaat atacaatgtt gtggggtcgg aacgctttat aattcacaaa |
| 42721 | tttgacaata ggctgcattt tcatatgccg aattgcctaa gagttctctt aaaggaacag |
| 42781 | caatgtgaaa gaaccacatg gtaaggagaa tttacgcatg tcctgcatac taotcctttc |
| 42841 | ccctgtgaat tgcataagac ctagatgatc ttgggccaag gaagaaaatc tggaagggtg |
| 42901 | tttctaccca ccatgtgcag agtacactac aattttgaga aaacggaaag accatattca |
| 42961 | aaggcataca gaaaagcctg ttctgtttac aaaggctgac ccagggcacc agggacactg |
| 43021 | tccctaagca gatcagttgt gggagaactt ttctacatta gcatgcaata agcaaaccat |
| 43081 | tcatagtgag gagaagtcaa actcccaagg gtccatttga taaaactttg tcattgtcga |
| 43141 | gtggaaagaa gatactgaga aacttacact aagtggtggg atcccttgtc tttgtctttg |
| 43201 | aatctacagg tattggagaa gaaattctgc ccttgaaatt tagactccat aaattaacat |
| 43261 | ggcacaacaa ataaagttca tggactgaat ttaggctcct gggatttttg agatagcttt |
| 43321 | ttatgtgaac tttgtaaaag ttagagtaac ttttaaagta gttgttacat gtgccacgtg |
| 43381 | gcttattgtt atcaagaata cttgaactat ttactgttca ctttgagtac tctaggttaa |
| 43441 | aatttgcctc ttttttgttc tacttcctcc accccgcagt ctatgtaaat atatacttta |
| 43501 | gatggacaaa caactgccta aaccttgcca ggtgaattaa agtttcagat tgaaKctttt |
| 43561 | ttttccccct taacaagctt aaagatatta ataattttaa atggtaaata taaaatacct |
| 43621 | gctctgtata gcttccccaa ttctaagaat gtaaaaaata ttacatgctt gttataaaaa |
| 43681 | ttgaagcatt acacacaaat ataggaagtc cattgtaacc ccacacccta aagatcacaa |
| 43741 | acaatacttt cgcctatggc tctaacacaa tcattttcat tttactgaat aacttccata |
| 43801 | tgcaagtata gcatcaccta gttgccatgg tagtgcatct actatatggt ttggttttga |
| 43861 | tattttcctc tctctctcat tcccataata taggaatgta ggaaatttta agaggacatt |
| 43921 | gacttgtttt atgatcattt gagctgtcca tagttagcct atccagtgga ttaaagatgc |
| 43981 | ataaccattg tgtctgggat ttgttcagtg ccatcttttc aatcattggt tacaatgaag |
| 44041 | ctgggagtcc cctctgaggt tcggcKttct gtggctcctc tcccctgcac tccaactttc |
| 44101 | tcaaagcctg agcctcaagt gatttcccag tagYtaagta aactgccccc ttcctccatc |
| 44161 | ttcaccaagt tcccaaagct Kttcagactc aggcttcaaa tctaacagga tgtggctcca |
| 44221 | cacattatat ttcagttgtt attttttaat gtggaaaata attcaggaaa gttttggcta |
| 44281 | atgcttcaag acatcatgca ctttatacac aggatttgtt cttaagaagg tagtatagtg |
| 44341 | acaggaggaa caatttactt cagaatatcc attttatgtc caagccatgc accagccact |
| 44401 | tttgacagac aggatttgag tatgatgata aaatggggta agcatatatt ttaagttcta |
| 44461 | acatctgtat aaataagaaa aatgaaaaat cactggaaca catctacatg ctagtgagaa |
| 44521 | aataattact aacagagtaa agtttgctct gtgaaactgt tgctcaaata aagaattatt |
| 44581 | cttgatatta gcatagagct gagaatattg cttttttcca aattcctagg tgagtaggca |
| 44641 | atatagctag ccacgatttt tcaggatcat ttgtaaagct tcacttttag gaattttaac |
| 44701 | cccaagtgtt ctaagacatc aagtaactga ccttttcatc ccgccgaaca aacctcaaag |
| 44761 | tggagttgcc aactatccca caggaaattg agtcaccaca ccaagcaaca cacctaaatg |
| 44821 | tcgacagcta gcaggacaaa aacagtgagc cagtttaatt ttggctttta ccaagaacac |
| 448B1 | atactgcatt cattaataaa atacttcccc tcaacccctg ccagagtagt atacactgtg |
| 44941 | gctctcctta aaaatacatg aaaactcttt aactgcacca aatcttgcta agtcaaggaa |
| 45001 | ctaaacagag aaaaacaccc aMgcaaaaac tattggctaa actccctaaa ggctgggaga |
| 45061 | gggctatgga acagcaactg agaaataaat tgagccttga aggcccctgc tacaacagca |
| 45121 | tcagttttct tcaaggcacc tttgaactat gtaacacccg cattctgctg aggctcttct |
| 45181 | gtgaacaaac agtcctcaat tcttgdgggc cagcacttgg cttctgtcac agtgatatgc |
| 45241 | ttagaggttc ccctgacaca tggcctgaaa ctgtgccagg ggctccagga gaagatgctg |
| 45301 | aagaagcagc ttccttttga aaaaggaaaa aagtaatcac ctcagagcca cagcagttag |
| 45361 | ctagaggttg aggtcatttt ttgtgaaatg ttcagtccat tactcttacc cacaactcac |
| 45421 | agtgatgttg accattgagg taaaaggatc cactgtacgg gatgtgtaac tatgtggaca |
| 45481 | ttagtttctt tgccaacact tgaaggtctg ctaaaggact gtatcggctt gtcaeaattc |
| 45541 | ctagatagac ctattggtat acatgaagag aacaagtcca aaaaatacca cttattttgg |
| 45601 | acactatttg tatgccctta atttaaaaaa aaaatacaaa ttataacagc tgaggtcggg |
| 45661 | cacagtggct cacgcctgta atcccagcac tttgggaggc caaggcgggc agatcatgaa |
| 45721 | gtcaagagat caagaccatc ctggctaaca cagtgaaacc ttggctctac taaaaataca |
| 45781 | aaaeactagc cgtgcatggt ggcgggcgcc tgtagtccca gctactcggg aggctgaggc |
| 45841 | aggagaatgg cgtgaacccg gcaggcagag gttgcagtaa gctgagattg ggccactgca |
| 45901 | ctccaacctg gacaacagca gaagactccg actcaaaaaa acaaaaaaaa ttataacaac |
| 45961 | tgatccagat tttttttatt attacacttt aagttctagg gtacatgtgc acaatgtgca |
| 46021 | ggttagtttc atatgtatac atgtgccatg ttggtgtgct gcacccatta actcctcatt |
| 46081 | tacattaggt atatctccta atgctatccc tcccccttcc ccccacccca cagcaggccc |
| 46141 | cggtgtgtga tgttcccctt cctatgtcca tgtgttcgca ttgttcaatt cccacctatg |
| 46201 | agtgagaaca tgcggtattt ggttttttgt ccttgtgata gtttgctgag aatgatggtt |
| 46261 | tccaatttca tccatgttcc tacaaaggac atgaactcat cctttttcat ggctgcatag |
| 46321 | tattccatgg tgtatatgtg ccacattttc ttaatccagt ctatcattga tggacatttg |
| 46381 | ggttggttcc aagtctttgc tattgtgaat agtgccacaa taaacatacg tgtgcatgtg |
| 46441 | tctttatagc agcatgattt ataatccttt gggtatatac ccagtaatgg gatggctggg |
| 46501 | tcaaatggta tttctagttc tagacccttg aggaatcgcc acactgtctt ccacaatggt |
| 46561 | tgaactagtt tacagtccca ccaacagtgt aaaagtgttc ctatttcttc acatcctctc |
| 46621 | cagaacctgt tgtttcctga ctttttaatg attgccattc taactggtgt gagatggtat |
| 46681 | ctcattgtgg ttttgatttg catttctctg atggccagtc atgatgagca ctgatccaga |
| 46741 | ttttttaaaa ataaaagaac tacaacaaaa agctcctaag agcaaatact taaatctccc |
| 46801 | taaccagttc cagcttctct tctcttaatt cttacaaaat ttcccctttg ttctaMggaa |
| 46861 | ggaattgata ctgtctacag tagatagaac tggaaaaaat gatttctgtt tcaccgtgta |
| 46921 | ttgctttgag ctagtcattc ctttagtttg ctacagtaaa agatttgtgt aggttaggtc |
| 46981 | taacttttgc tgacttaatt ttttttcctc tatacatttt atctgatggg aaatacatga |
| 47041 | aggctttgga ttctctttca atgtgtacat gtaataggtt taagcagagt ttgctagtgg |
| 47101 | caagctgctc tcatctccaa gtgtatttct ggcatcaaat tattgttttg aagtatttta |
| 47161 | aaattgtata aaatatataa aactttattt agtacttcat aaagcttcca aaggaaagat |
| 47221 | gtgtgcaatt ttgtgacggt tttctttttc ttttatcttc tcactaacta caattgtttt |
| 47281 | tccgaatgtt gcctatgcct ctagtagaat ttttatatga aataaacatg aggttggctt |
| 47341 | tcattgactt ttagtcccac cagtaactca acatttattt agcactgact atgtgccatg |
| 47401 | cactttatta gaggctgcag aaacaagatg atgaaaagag tgtgtccctt acccagagtc |
| 47461 | cagcagagga ggtaatttca gcaaacaact gtagagtgca ctctcacaga aggaagcatg |
| 47521 | tgggctatgg gacagtaaaa gaggactatt taaatcagac agggacagag gtggagtggg |
| 47581 | aaaaggctga gtcctcaaga atgaaataaa agagagtggg aaggattttt aagagaaaag |
| 47641 | atgagcaagc acacagacct atgatgaaca agatgtctgt gtcggccagg cgtggtggct |
| 47701 | cacgcctgta atcccagcac tttgggaggc cgaggtgggt ggatcacctg aggtcgggag |
| 47761 | ttcaagacca gcctgatcca catggagaaa ccctgtctct actaaaaata caaaattagt |
| 47821 | cgggcttggt ggcacatgcc tatattccca gctacttggg aggctgaggc aggagaatcg |
| 47881 | cttgaacctg ggagatagag gttgcagtga gcagaggttg cgccattgca ctccagcctg |
| 47941 | ggcaacaaga gcataaaaac tctgtctcaa aaaaaaaaaa aaaaaaaaaa aagatgtgtg |
| 48001 | tgtcaaaaaa gaaataacca tgagtagggc cttgcttagg ggaacagatt ctggagaaat |
| 48061 | ctgagaaatc ctcagccact ggccatctca aagactggcc atgtcaggca tctgcaggta |
| 48121 | ggactatatc ttcaagagta ggagtcacag agtgactcag agtactgtgt gacatgggat |
| 48181 | ctgattttat tttatatgga aaattctagt gctatgcaat taatggaatt Kaagaaagta |
| 48241 | aagttgaagt cagtgcaacc attatctttg accatattaa gaataaaatt aaataactta |
| 48301 | tagaccattt gcttctctga cgattattct tatctaagac aatctctgta ttaaaatcct |
| 48361 | acgcagacat atgttcccac aatatgtcag gtatgaacag aaggactgcc caatgtgact |
| 48421 | tgaaagggag tggacttctt gctcttaaaa actacaacct tgtagtcact aacttattaa |
| 48481 | tcatactgta ataagatgga gaaaaaaaca atggaattca ccagaatttc tttcacgaga |
| 48541 | gtctgcctgc attaaaatgt tcgataaagc tatttatgac agaaggtgag actttgaaga |
| 48601 | gctttggttt gggtggaact aatctgtgcc tcatctacac aggaaagggg aggaatatgc |
| 48661 | cagttgctag agagggaata tgtatgtgtg tgcatgtgtg taagcgcatg agagagcgag |
| 48721 | agaatgtgtg tttgtgcaca tgcatgtgtg tgtgtgtaag agaaaggagt cagcttggtc |
| 48781 | tctgtgtgtg catgtatgta agggcatgaa agagtgagag agagagagag aatgtgtgtg |
| 48841 | tgtgtgtgtg tgtgtatgtg tgtgtaagag aaaggagtca gcttggtcta aagggtaact |
| 48901 | ccaatttgta catttttcct ttctcccaat catcaataag gttattgaag atgtgtgtgt |
| 48961 | gtgtgtgtgt gtgtgtgcat ataagagaaa ggagtcagct tggtctaaag ggtaactcca |
| 49021 | acccgtgcat tttccctttc tcccaatcgt caataaggtt actgaaggta caaaatcccg |
| 49081 | acttgttttg ttttgtttta ttcattgaag ggactgagct tgaaggccag attggcccaa |
| 49141 | gcagaaatgt ctctctgagc tgacataaga gaagagtcaa agaaagagga gaaagctttt |
| 49201 | ataagaataa ggaggccttt tgtccaaaaa attggagttt gagctaagtt taattaggca |
| 49261 | tctaggttgg gggcttcatg tgacatgtgt gcaactgtga gtgatattat tctttgaaag |
| 49321 | aaactggaag agtggagaaa gattccaatc tctattccac ttcatacaag caattccagg |
| 49381 | caattcagct actaatcttt ctagggcctt atttcaccta tagattggag ataataacaa |
| 49441 | gagccttgta agttatttta gttcaagtga gaggttgtga gttaaataat cttgcattga |
| 49501 | ttgcctaata gattcttttc ccttcctcct ccattgtatc ttcactttct cctggactca |
| 49561 | agtctctaac ttaaatgcaa acattctatt aattttagtg agcctatcaa ctaataccta |
| 49621 | ggtataacct cggggtccag aaacttaata gWtacctgga acagttttta gggtagttag |
| 49681 | aaagaaaaaa gaatttcaag gaatgaacag ggattcattc tgaagaagtt cacttagaga |
| 49741 | acacaggcca ttcaggaagc tacaaacagg tagggatata aagtttagat cagacagtgg |
| 49801 | acaaagataa agatggagaa ctagtcttgg aggacttgta tactattcta agacaatatt |
| 49861 | ttcttctaaa gaggccatta aaaatcttct aagtgaggaa gtcacatggt cagatttgct |
| 49921 | tttcaaaaag attactctgg gaatagtata aaggcagatc agaaaagtaa agaccagtta |
| 49981 | agaggttggt gaccagataa ggagaaaatc ctaagcagtc cctgaagggg aaggagaagc |
| 50041 | tgcagaaagt taggaggaaa aactctcagg agcaaagaaa aaggaaatgt caaaggaaaa |
| 50101 | tattgttatg gactgaatgt ttgtgtctct ccaatattca tatgctgaaa tctaatcccc |
| 50161 | ggtgtcacgg tatttggagg gaaagccctt cggaagtaat taggtcatga gaatggagcc |
| 50221 | ctcatgaatt agattcgtgc cattataaga agaggccaga ggttgctctc tttctaccat |
| 50281 | gtggatacat gctatgtggg cagtctgcaa ccctgaagat gccctcacca gaacctgact |
| 50341 | atgctgacac cctgatctca gacttccagc tcccagaact gtgagaaata aaWgtttgtt |
| 50401 | gtttaagcca tcagtttatg gtattttgtt atagcagctt gagctaagac aaatatcaag |
| 50461 | gctttgccat tgatattcag tacagaccgt atatacccac aggtttgcgc taagataatt |
| 50521 | ttcattccaa acacatattt cagcaacaca agactaaaga gggtgttaaa gaatgctact |
| 50581 | gaggaagctt ttgtctggga agttgtttga acaaagttac aggacaattt ttgcaaacag |
| 50641 | gtttaaaaaa agttacaatt atgtatgcta tttacgtatt cttgttgtaa ttttaggaat |
| 50701 | caattttatt tccctggggg gactgtagga gagcatctgt gtttccatca tatactatca |
| 50761 | atcttaggat ataacagtct tttgttcttc acctttgtca attactgcat acagtattcc |
| 50821 | ttagagaaaa caaggagttt gaacagatta tctttagcac ctacccagct ctaacattca |
| 50881 | tttattatct gatttctgaa attgtactat ctgggttagg tgttggtttt aaggttgttc |
| 50941 | attatagtta gcctggatgg ggtcagctat gtttgtagag ggaattccta aactgaccag |
| 51001 | ttacttgtca aggatcccat ccaataccat gatcctggaa ttctgtgagt ttagctcaac |
| 51061 | tgttcttcag gggtacatgt tttctatttg aaattgaggt atYctcagaa ataaagctat |
| 51121 | taataaatag aaagttctgg agtatattta cactcactct gcttatctcc actcctaccc |
| 51181 | ctaccatatt cccatccaat actttgccag ttgacttacc aatttcttct taatttctgt |
| 51241 | aactgtcacc ttcagtcaaa ttctcatctg tgtatttcta gattcatata atggtctact |
| 51301 | actggatctc cttggattca ctttttctca acacagtcct atttataaag tcagaataat |
| 51361 | cttccttaaa ccctggtttt attgtttcat tcttttaaga acctgctgca caactctctc |
| 51421 | ctacttattg tattaatgtc acacttctgt tcacataggc ctttttcggc tgcttttcaa |
| 51481 | tacctttccc ttttatttac atctaagact atcccatcca agactgccca tcttagccaa |
| 51541 | tctttccacc tggaaataat ttgctggctt ctgcctgtat gacttttcta atgtcatttt |
| 51601 | tatatttgtg tctaggcctt tctataactg tctagaaaat tcctgacatt tacaggcact |
| 51661 | cagcaaatat ttgtgagtga ataatcagta agtatgcttg ttttctatca gataagaata |
| 51721 | tttatttcca aaYgagattc aaacagtaat actggcctgg gtgtggcttc ctacatgtaa |
| 51781 | tcaccctcgt ctatctcttg ttctcagtct tttgctttca aactctttct ctttttttaa |
| 51841 | aagagtatta ggaactaaca ttgccaaaat ttggtaatgg tcaagcagat catggtgtct |
| 51901 | tactgaagtg aaatattaac attaaaataa aaatgacaag tattatggta tatcattcat |
| 51961 | atgtaatgat ccttacaaaa tagagtaaaa ttttaaaaga attcaccaat acaccagtat |
| 52021 | gatgtttggg agaaacagat gaatgcacaa aggtgcatct gatgcaaaca cccccagaaa |
| 52081 | aatgagcata agttcatatt atcaatattt ttcactctaa aagtgttcac tgacttccct |
| 52141 | gaagtcacca aggactccaa gttaagaata ctttcctgag tgaagaaaag aaagcaatta |
| 52201 | ttttaatcaa cactgcccca gaatctttta ttcggaatcg ctatctctgc tttcttcttc |
| 52261 | ttcttttttt taactttctc ataaatctct gtttagcatt tatatctctc tcactatgag |
| 52321 | atacctaagg gttaagaagt aatgccaaag agacatttgt ccagttgaag actgggatta |
| 52381 | agaaaaaaca cacagccggg catggtggct catgtctgta atcccagcct tttgggaacc |
| 52441 | caaggcaggt ggatcacctg aggtcaagag tttgagacca gcctggccaa catgatgaac |
| 52501 | cccatctcta ctaaagaaaa tacaaaacat tagccacgca tggtggcggg tgcctgtaat |
| 52561 | cccagctact tgggaggctg aggcaggaga atcacttgaa cccgggaggc ggaggttaca |
| 52621 | gtgagccgag attgtgccac tgcactccag cctgggcaat aagagcgaaa ctctgtccca |
| 52681 | aaaaaagaga aaaagaaaag aaaaaacaca caaaagattt cccgaaaaga ctaggccctt |
| 52741 | ttaaacaggc tatgaacaga gtctcagaaa ggtaatttct tatgtacaat ttttaatctc |
| 52801 | attacttgca ttacaagcaa cacatatgag tgataaacta ttttggccta ctggtaaacc |
| 52861 | tacatttgtt aactgtgtgt gcattataga gaacaaattt cagaactcaa gagtatatcg |
| 52921 | gttatatttg gcttatccag ttgtatatga actggctcca tacacgaata agataaatta |
| 52981 | gttttataat tttacaagaa tatctatgga gatttagatt tggctaagaa agttattata |
| 53041 | cacacagaag aatccacaac ctataagagc tcttcatggg aaatagtgtg atagatcaga |
| 53101 | aagagcatgg acttttgagt cactcagaac ttgaattcca gccttgccat tgttctagtt |
| 53161 | gtattatctg aaataagcca tttaaaagac ctgagcacat tgtatacatt gagaattaca |
| 53221 | gggttattat gagaaatgca taagaaatgt aaagtaccaa gggctgtatc tgacatatag |
| 53281 | aaggtattca atcaatagta ggtatactga accactttgc agagttgttt aaataagtta |
| 53341 | attttaaata aggtagttaa tatttgagag tttccttcct tttccacaga ttctcaatcc |
| 53401 | taaaacagtt actatttttc catcatcacc atcaaccctc aaatattaac cagttctgag |
| 53461 | ccaaatgtga gtgaacaacc tatgatttag aatagctttt agaccaggca cagtggctta |
| 53521 | cgcctgtaat cccagcactc tgggaggctg atgcgagcag atcacttgag gccaggagtt |
| 53581 | caagaccagc ctagacaaca tggcaaaacc gcatctctac taaaaataca aaaagtagcc |
| 53641 | aggtgtgtgt ggcacatgtc tgtagttcca gcttcttggg aggctgaggc aggagaatca |
| 53701 | attgaaccct ggaggtggag gagatggtgc cactgcactc cagcctgggc aacagagtga |
| 53761 | gactctgtct caaaaacaaa caaacaacaa aatagaatag cttttacaag accacaccaa |
| 53821 | atcatgaaaa gtcaactatg taggaagaat atgtatttgt cattggttaa tgacagagca |
| 53881 | tcacttacac tgaggttact gggaagatca tttcaaacat ttcccaaaga atgttccaat |
| 53941 | caatattgtc cttataagat tctccttact aatgacgttt tgtggtcaaa caagcttggg |
| 54001 | aaatattgta tatgtaggct ttttacagtg atttctaatg gacacgaacc taccaaaagc |
| 54061 | tagaaggtcc acagcaaaat gagatattta attttattaa caagcattat ctaaacctgt |
| 54121 | ttgattttag aatgcatttt tcacacaaca actatcacct cttgaagaat acctggaata |
| 54181 | ggttttccta tacctatacc tatatgtcag gtaactccaa aacattaaca aatacatgtg |
| 54241 | tttagtaggt tcccatatgg ttgaaatctc cactaacgac attttatttc tcttcctcct |
| 54301 | gactttgtat gctatcgaga aatggtcaga gagtaatctt cctccctccc tttcttcttt |
| 54361 | tctttctctc tttttccccc tgaaagagaa tagtcaaaaa atccatacat atttattgag |
| 54421 | cctctctgat gtgatagttg ctgtgaaata caagaacatg aaagattcag cagcctgggc |
| 54481 | ccaggaagga accataataa cctgctgaga aaatcagaca catgatcggc tgctgcctag |
| 54541 | agacagtgta aggtagcata taaatcattc agcctgggag tgctactgta gttcgagcag |
| 54601 | ggagagggag gtatggacta tagctcagtg tgctgggttt caggaagaag gtgaacttgg |
| 54661 | atgtaggtct tcaaggaaga ctgtggtttt aatggctgtg ggaggaggga gtccgctggc |
| 54721 | attgttttca ggYgtaggaa atgtctgcag cctgaataaa ggttgtatgt gtggacagta |
| 54781 | aatgggctgg gtggagtaga cacattccag ggaggagaag ggatatagag ttgaacactt |
| 54841 | aaatgtgtgg ccttgagcta gtggatattg tgggaattgg ataagtgcac tggtagccta |
| 54901 | agaggataga atggagaaga gagaaggatg gagactaagg agctattgtg gtagtccaga |
| 54961 | tgcagtggaa agcaaagagt tgtggaggta tccactttag tggtctacga tgacagttac |
| 55021 | aagcaataga aagagtatga cttgggaaca gtggttcaaa tgctggctct gcaacttagc |
| 55081 | aaccatgggg caaatttggg caaattaact tctctgtacc tcagttttct tttttgtaaa |
| 55141 | atagggctaa aaatacattc cctagaaggt ggcctccagg ccattttccc acactttatt |
| 55201 | tgaatgttaa aactaacttt aatctaaaaa tcatatattc atatctctat cttttttggc |
| 55261 | ggttcagttc aacttaaatg tgttatttgt taaaaacttg taattatcat ctattacatg |
| 55321 | taaaatataa gagctgcttt taatagtaat tcatctcctc cctatctttt tttaaaacat |
| 55381 | ggtcaaaatt aacaaaacaa tcttgtattt ttgatactag tagtggtatc actactacta |
| 55441 | gaggtacaag gactcttgta cctctctgaa aagtagcata attagcaaca gtattgatgg |
| 55501 | tatctgctat tttagattat ttaaactgta gtcactaagg taagcaaggg gaagaaggca |
| 55561 | tgtcatttat gtgtttttca tccttaagat tagcgtttgg aagcatatat tattccacag |
| 55621 | caatctggat aggtcttgcc atgccctgat cttgtaagcc caaattttat tttgaatact |
| 55681 | ccagctttgc acttaaatga cagctccttg ataaaagaaa aacaattgcc taacaatttt |
| 55741 | cttccaattg tctgaaaaca taatgggtgt acatacacac acacacacac acacacacac |
| 55801 | acacacacac acacacacaa tctctagggc atatttacta gatagtatta tgtaatgtgt |
| 55861 | agacattttt aattatgata attaagacaa aaatatgtgt agcaagtttt tgatttgaga |
| 55921 | caaatttgag atttactaaa attattatat gtgtgtcctt ctgaagactt catattttca |
| 55981 | ttatatatct tcattcttga agtataactc ttaaattgtt gaattaagtt ttatgaatta |
| 56041 | agttgttttg ttttttgttt ttttttgttg ttgttttttt tttttggcca ggcacggtcg |
| 56101 | ctcatgcctg taatcccagc actttgggaa gtcgaggtgg gcagatgact tgaggccagg |
| 56161 | aatttgagac cagcctggcc atcaaagcaa aaccccatct ctactaaatc taatcttgtg |
| 56221 | cctcaagcga ggcacaagac tcacttgaac ctgggaggca gaggttgcag tgagctcaga |
| 56281 | tcgtgccact gcactccagc ctcagggaca gagtgagatt ctgtctcaaa agaaaaaaat |
| 56341 | ttttttccat ttttaccaat accactattc tcaattcatc cactatttca ctaggaatta |
| 56401 | ctaattcagt ttacataatt tattcaatga ataaattatt caatgaagaa gcttctgggt |
| 56461 | atttaggtgc tacatgctaa tttcaattgg catattttgt ttttcaaatt tgttcttggt |
| 56521 | gacccaataa ttatacctgt ccaccttgaa agtgtctttc ttcatcacct tcaaggccac |
| 56581 | cgctaacaag acctttgaaa agaagtaaac agtagagaat ttcaacttct gtgatgacca |
| 56641 | ccgtcttctt ctgaatcccc agactccact catacaaggt tttggggaag tttaaatgaa |
| 56701 | atgaagcagt ggcttacaaa gtttattgac agtaaactac agtaaaattt atattttata |
| 56761 | ctataacctg gtagatttgt ttaaagtaag aggtgctcac ttttgaaggc atgtggatgc |
| 56821 | tggctttcag taatataaaa aaaatgtaca aaaaggtttt atacctatat ttatttttaa |
| 56881 | aaggtagagg agagcagtgg actcctattt aaagaaactc ttgtgtttta taaataatag |
| 56941 | tcatggcaaa atttgtttac agtctttaac aaaccatgca tttcagtata aattaaacca |
| 57001 | ataaaaagaa ttatatatat aaaataaaaa ggaaatgtat atataggaac agtatatacc |
| 57061 | atacacataa gagtaaatat atattcgagc caatacagag aattgaaaca taatttaaca |
| 57121 | tgaaataaca tttatcctta ctatgtatga ttcactctga tgttttctat ttWatttaat |
| 57181 | tttaaagtat taattgtgac cccttaaatt gattttgaaa tctaccaatt ggtttgaaaa |
| 57241 | cactgaggta agaaattacc ttgcttaata aatgttagtt ttgtttccct cttccctctt |
| 57301 | cctccctttt tataattaaa aaactctccc tctacttctc tacttcacgt gagagtagaa |
| 57361 | cacataaaaa gatttcagtt aactactgtg catatttttt gaagtaattt ctccataatg |
| 57421 | tgtgagtttt ctcagggtac tacaagttta ccaacagaac acataaagca gcggccactc |
| 57481 | actgaaatgc ctataggaga cagatagacc atatgaatga atgaatgaca tggttgctgg |
| 57541 | gcatcctggt tgcagtggct agaactgtgg agggtaggag cagaccacca tatatcctag |
| 57601 | gcatttcatt ggcaaatgaa caggagaagg acacctttcc gtttggccct cattttccta |
| 57661 | gatctttcct cccttcctct gtcaatccct cctacatagc ctatgttccc aaagcacccc |
| 57721 | ttgtattata ataataatta ttatgatatc tgtccttaat caagaactta tactggattt |
| 57781 | ttaatgcctt taggtcttcc ccaaacttca gttcatctat tcgaagtccc tataaaatct |
| 57841 | acctccttag acttgtctgc ccccattatt cccctgtggt tgacctcacc tccatctcta |
| 57901 | tcttttgtca gcaagtttcc tcagagttcc ctctctgcat acttgttcat gctgggttat |
| 57961 | ttgtttttgc cacttgatca tctagacctt tttccctctt tccctccttc tattcaattt |
| 58021 | ctgcctttca ctgctgtaca aaggaggggc agggaggccc tccagggtgg agggaatcag |
| 58081 | tcttgagata acegagagat tcagggtgat gagtcccaaa gttgaagaga atccacatac |
| 58141 | ttcttctgtt tttgttttac ctagcaagga gccttttcta tagacaaagt tRggaaacca |
| 58201 | gaggttatga cgtgattcat ctttgcctac ttaaacttta gcttgattca tcacttctca |
| 58261 | acaaatactt gttgaacccc accagtaata cgagaagctt ggtattctat gaggtgctca |
| 58321 | acttccaaag cttagttcag aagaattctg gcctcaaggc gtctaccatc tagaagcaaa |
| 58381 | caactaacaa atgaacaaat gcagtgattt caaaagaaat taaggtgtgc tgtggtagag |
| 58441 | gcatgcacag gtcagaaaag ttcagaggtt ccagacaggc ttcctgggga ggatatccat |
| 58501 | gccccScctc cacctggtag ttcatgatca gagtttgcaa gcttggctgg gatagaggga |
| 58561 | atagcgtgtg aaccacagaa ggagtggatc tggtggctca gacacggcaa gaaccagctc |
| 58621 | atggcactag gctgcaggca caccatgcac aaaacagtct tttctttccc cagttctcca |
| 58681 | aattctctaa agtggaagcc taggggtgag ggtaagaagg aggtaggaag ctacaaccac |
| 58741 | tggagcaaag gacgaaagaa ggggcttgag tgtctaaaag tgcagaaact caaccgactg |
| 58801 | gacgctgaga caatgtttaa aaaggagttt ttttgtttgt ttgttttctt gtgttttttt |
| 58861 | ttactttaag ttctgggata catgtgcaga acgtgcaggt ttattacata ggtatacatg |
| 58921 | tgctatggtg gtttactgca cccatcagcc caaactaggt tttaagaccc gcatgcattc |
| 58981 | ggtatttgta ctaatgttct tcctcccctt gctcccccaa cccccaccag gccccagtgt |
| 59041 | gtgatgttcc cctccctgtg tccatgtgtt ctcattgttc aactcccact tatgagtgag |
| 59101 | aacatgcagt gtttcgtttt ctgttcctgt gttagtttgc tgagaatgat ggcttccagc |
| 59161 | ttcattcacg tccctgcaaa ggacataaac tcattctttt ttatggctgc atagtactcc |
| 59221 | atggtgtata tgtgccacat taaaaatgac tttttaaagt ggcaactgtg ggaggcaaag |
| 59281 | gagaaaaaac aggagctgcc agataaggga gtgaagagaa cccagcactt tgagaattcc |
| 59341 | gttacatttt gcagcttgaa ttcttcactt tttgctccca ttatgtgcct ctttctcctc |
| 59401 | tccatgaact gggccctgtg gacccaggat cctgctgtct ttgcagggca aagaatggtt |
| 59461 | agaaatgaat tcattaaggt gagaaaccat tgctggcata attctccttc acggaaacct |
| 59521 | ggcacttaaa tttaacctgt ttggttagat ccagctctgc ctcccccatt tYtttctgca |
| 59581 | gaaacaaggc tggcagatcc ctgttcctct taatccttga atggaccaaa tgctgggtta |
| 59641 | tgctggggca gtgagggacc ttacaaacac tgaaatttaa gagaacagtt tttggagtaa |
| 59701 | ggcagtcctt gtttccaaat ctgagctttg ccactgaYtg actagcttgg caccttataa |
| 59761 | aagtgaatta acctagcttg ttttctaatt tataaaatgg agaaagcagt acctatatca |
| 59821 | cagtatgatt ctgaaaatta tttaaatagg actatgtatg taaagcagtg cctgaaaaat |
| 59881 | agcaagtggt cctaaagtca ctatgatgat gaattttata tgcaggcacc tagagcatgt |
| 59941 | taggattttt gggatctttc ccagctcaca ctgaagcttc actcttctcc aacagtctgt |
| 60001 | tatccaatgc tggataactg agatagtctt cccagcattt atcaatgtgg ttaggaacag |
| 60061 | taatcctgga aagcattctc aaattattta tctaattaaa caagcaaacc tttttagccc |
| 60121 | agacatccta aatctctgta gcttgtcttc gctgatttgg tcagaatagc tcagatggta |
| 60181 | ggaagggctt tctgggtaga aaaaaataaa ttagcagtct ggaaaacaaa agatctctgc |
| 60241 | ttaaaagagt ctctaaaaca gcctgatcag cctgtgtatt ttgccatatt atctagcatt |
| 60301 | agtaggggac tgggaaaaat ccagggctcc ttgtagagat agagactggc aaatattatg |
| 60361 | tttgatgcat gagaaccaaa tcagaagtta actctttaag ttattaaatc actaaacagt |
| 60421 | ctatcacaat tatgtgttta ttactagact gaatttctct ggggctggaa ccataatgaa |
| 60481 | ttcatttttt gatctctggt gctaaacact ggctgtcata tactaggtat ccaataaatg |
| 60541 | tttgttgaat ggaactgaaa aagcatgaaa cacttcccct gttaaggctt cttttctata |
| 60601 | atatttccat agtttctaat gcatctgaga atcattaact ggactgttag aggaatatga |
| 60661 | atatggttga gggagctaca aacttcatgc tctgagatgt cacatgatta gtgtaacttt |
| 60721 | tgttccaggg gagagaaatc cctaagagag tgactgttct ccatgcccac ccacccacag |
| 60781 | tgtcctatag agcctttcat tctccagatg cctataatga ctatcacatt gatttgttta |
| 60841 | catatctatt gccttgtctt ccttataggt acagagtcca tacatcctaa ttggcctgga |
| 60901 | acttccaagg tctatgcctg ttataccagt gtaattatga atagcattgc ctttcattct |
| 60961 | aaaaagtgtt ctggtttgaa tgctaaatca tctagacacc ctacttatga gagggttcac |
| 61021 | tgtacatcgt ttagtccccc aactcccctc cccttggaaa tctgggtact accctccacc |
| 61081 | gggggctttc cactatggag gcagctctct caatcacagc attcaggagg tgtaagtcca |
| 61141 | ctcccaacag aacagacacc tgtctacaaa gagactcttg gtggaagaaa aaaaaaaatc |
| 61201 | acttccgtaa gtttaagtct tccttttaaa cttgggtctc ttttgcacat cagaaacaaa |
| 61261 | gaatctctaa taattacatc cctaaatgta ctgaagtcta gaagacatgc ccatttgaaa |
| 61321 | actctaatct agtaattaaa gcttctcagg ttaaactgca ccttaataat gtagcttatt |
| 61381 | ttgtcaagat cagttaaggc cctgtaatgt gatttggaat aaatggacag acagaaatta |
| 61441 | aaatgtgctg gacaaggtaa tcagaaatct cacttcattt agtcaataac aatggcaatt |
| 61501 | cagcgggttc cttttactac tgggatgtaa ttgtgcttgg ctttaatttc ttgtaatgat |
| 61561 | ggaacaaatt aaattacttc atacttttag gaacatttgg ctacatcatg ttttggYgct |
| 61621 | tccataaacc ccactggaaa ttaaatcagc tctgtggttg ctcattctcc ctctttagtg |
| 61681 | ccatgtctgc cttctgtcag caaaatggta atattgtcta ccttaatcca ccagatgcag |
| 61741 | ttgagttaaa agaaattacc tctcccacct ctttcctttc aaatctccct atttgtagct |
| 61801 | atcaaatttt ggcatgcctg ctggaacaca gtcttgttag aacaagcacg gtaattctct |
| 61861 | cgtgagactt taggactctc tctacgacaa ggtcagaatg tacctgtgat tcaaatttag |
| 61921 | atgaggatat gagttggtaa gaacatgaag agcatcagac agttttcagg acacccacta |
| 61981 | cttttatttt gtgctatcag tttttccttt cccaaattgc aggtggagat gcaatcttta |
| 62041 | acgctggaaa tctcttgcta tatgccatta aaattaatta gtctagcaca ttttatcttt |
| 62101 | aggcccttac ctagggcagg taaaatattc tagcacaaaa taacacaact ttcctgaaca |
| 62161 | ccatggctac taagttcata tggtaaagat tatactatac attaccttgc aaatagagtg |
| 62221 | ttgcagactg atattaaata tttaaaatgt atgcaggatt attaataggc aacattgtgg |
| 62281 | cagagactgc taaatgtccc ctaatattga tgcttttctt catcacttgt tttcaaatac |
| 62341 | ctgttttcat gtaagcatgt gactgcccaa aatagagaaa attcacagcc tccttggttg |
| 62401 | ccaggtgttg ccatataact aagttttggt gaatgagttg gtagtagaag cgttatgtag |
| 62461 | atattatatg atgtgctaga aacttccctt aagagaaatt tgtcatctat ctttttatct |
| 62521 | gcctgtattt attgttttct ctttcctact ggctagaatg tgaagttgat ggctggagtc |
| 62581 | ggagcagcca tattggatgg tgagttgaat ttagcaacag aggctacacc tcacagtgca |
| 62641 | ataagatgga aagagcacgt gtccttgaag actgtactgg atagaggtgc catattaacc |
| 62701 | tcggactgcc aactttcaga ttttacatga aggagaaata tctgtcttcc ttaagccact |
| 62761 | tctattttaa atctctctat ttactgctga ataaaatctc aactaacgca aacaccaata |
| 62821 | acaaaaaatt cctattcact catgatatag gattatacct tagtcttttt tgtgtcatat |
| 62881 | atctgttcat tcaccatttt tgctttgatg ttcaataaac atctcaaatt taacatatct |
| 62941 | ataaaaattt tttaaatttt acgttcaggg gtatatgtgc aggttagtca tataggtaaa |
| 63001 | ttgcatgtca tggggtgctg gtgtacagat tatttcacac cgaggtaata agcatagtac |
| 63061 | ccctacagct tagtcttaag aagaagtatg ttcacatagg ccaggctgag acttggatgt |
| 63121 | tggatgactt tcttccagct tactctgatt tgaatatatt gtgtggtagt agatggctaa |
| 63181 | actgcttgct ccaaaactat ctgacaatgc tttttagaaa gtctgccaaa caaaactaca |
| 63241 | agaatcaccc tggagcttcc cttaggcttc agatatttgt cttccctgcc ctacttggaa |
| 63301 | atcattccag cctggaagtc attgtgagga accaaaaaat ggtctcccta ccatgaagaa |
| 63361 | taacactgct tccattgttg ggaggctctc cttcctacag gcactgtccc tcactcatta |
| 63421 | gacacttgaa taataaggat gttcatatat ttattggtgt tggtatcagt tagacagttg |
| 63481 | aatgatacaa atgttcatat atttcttctt attcaatagt ctcacaagaa cactattctt |
| 63541 | gtaaagtcat taaaaactgg atggtctttt cataattatt tccatgattt gtcatctaaa |
| 63601 | gccagtgttc tctcctcctg tcaatctgat agctttaaag ctaagttctg ggtgggtcca |
| 63661 | agtcatgcct gaaaggttta tcaaatgaca tattgcagtc atatggtcgg agcacaaaca |
| 63721 | cctctgaatg tacaatattt taaagcaaaa cagtttatgt gtaagctcta tgaggttacg |
| 63781 | gaccatgact atcttattta ttaccaaatc cccagctcct agaacagtgc ccctcaatat |
| 63841 | aaatctgtca aatgaataaa cgaagagttt gcctgcagta gtaaacttgt tatttgcaca |
| 63901 | cactcgaggc ccacctctat gtgtacattg tcagtgagtc agcctcgtgg atgactctgc |
| 63961 | aggactgtct gtataacatS cactacagtt accaggggac ccaattttca taaattgctc |
| 64021 | cagcatcaca ccctcctctt acagagactc ttcctgtcca ttctaatccc tgacacgttg |
| 64081 | tcatcctcac tcagattccc caatagatag aaatgctgtt aaatatactt gggtagagag |
| 64141 | ggataggcat gctcaaactc Sttttgggaa aaaaaattgc aactgtcaga aatgtgttag |
| 64201 | ctatcttcac agattatgaa acataaatat caattatctg ttcacagcag tgaaagcctg |
| 64261 | catatttttt taagatagca agtataaaac ggctataagt accatgttga gtagagacct |
| 64321 | accaccgaag tagagacctt cactgaaata gtgaagggat ggttgtgttg cagggtcaga |
| 64381 | atgattaccc actggttgac actccctcca ggactaaagg cagcatcacc agcgtccact |
| 64441 | ctgcttctcc actgcttact gttaaggagc aaggcaatag cattatctaa gtaggaacta |
| 64501 | cttcattctt tcaaaacctg gaatcggctg atcagaagaa gaaaaacagt ataaaaatac |
| 64561 | agaaagacaa gattctctgg ctgtcctaat gaatgctagc tagctataat agcatggata |
| 64621 | agacatataa ccaaacatat aagatctatt aattaagggg aatctctatt tgtattaact |
| 64681 | catctatttt ctctaaaacc agtatttagt tatctcccat acattggcat attgtgtata |
| 64741 | tttgcagaat tcaaaatgtt actgatgtga cttttttcca tcagacatct gagctatccc |
| 64801 | tcaactaata gttgagctgt tcctctatag ctcagtatct gagttagaac tcgaagtaaa |
| 64861 | aaggtataaa taagtcatgc tataaaattc aaaggcaatc agatgtttga gactatagtt |
| 64921 | atacagaaaa gtgtccttgt tcttgggagg aatacatcga ggagtttagg gatagagggc |
| 64981 | tatgatgtgt gcagttttct ctcaggtggt tcagagaaaa aaagacaatg tgtgtgtatg |
| 65041 | tatatttgtg tgtatgtgca catagagtga tactgcaaat gaggtggaat gtttacaatg |
| 65101 | agtgaatgca tgtggttttt tttttttttt tgcactaccc atgaagcttt ccagtgtgtc |
| 65161 | tgaaataact tccagataaa acttttttta aaggcaatca gaacagtggc aatgaataat |
| 65221 | tcaattattt catatttgag ctgtctttcc tgccaccccc accccctata acactacata |
| 65281 | catacatagg ctcacataag tgacacttac tatacttttt tgcctatgta atgagtgttg |
| 65341 | gcgaagtacg tgagtgggaa attcagtctt ttgcattcaa gctctaagta attctaagag |
| 65401 | accttttaga taatgatttg acttttaaga caacaatttt gaggttataa tatgatgtca |
| 65461 | tttcatataa acagaactgc ccttttgtcc tttcagattt gattgaaaat ggcccatgga |
| 65S21 | gtacaaccac ctgggaacat atcctcgaga aagacagttg aggcttattg cctaaaccac |
| 65581 | aggctactga aaacttggcc aaaggcgact actctagact ggtgacttca aaagtgctga |
| 65641 | tgaatacagt gaaccatgaa agatgcatta tttgttttga acattttact atgattgatg |
| 65701 | acccagattt ggcaagttag ttcactccca gtggagatga aagtttcact gccttctttc |
| 65761 | tttcaacatt cttgttgaga cagatttcac ttttattggc caatgtacta acaaatcact |
| 65821 | tgcatttgca gcataatgaa ttcattagca acactgtgga tcaatgctat acataatatt |
| 65881 | cctgagtcaa cattaactct taattaaact tccttattat aatgcctctg gaaattttcc |
| 65941 | ttcaggctac taaacttcaa gccaacagtc taagtgttaa tcagtaatca tgctagtaac |
| 66001 | tttttgagat gggtcagggc cagggccagg gtcagtgatc catcatgaat ggcaggttta |
| 66061 | aaaatggata tacgtgatca aaaataccca taagttgacc aaagcattct aacctttggg |
| 66121 | ttattctaac actcagtgta tcagccttct gagcaagcca attttttatt gccttaaaaa |
| 66181 | gtctaaaaga aagataaaaa cagagataat cagttttgtt tggcaatggc aggggtgtgg |
| 66241 | gaaatgaaaa agaagaaagt taaagatgag tgaaaatagg atagtcttgg cactttggta |
| 66301 | tgaaattgaa gtacttattt aaactgatga atattgtaca gaaatataca gagagcagtg |
| 66361 | aggggctaag aacctaattc caaggtaact acaaggaaaa aattattgat ctggattgtg |
| 66421 | accagaattc aagagtttga ctgttgtcat tgttgctgtt gttattaagt taaaaaaaat |
| 66481 | gagaaaaaaa tatacttttt tctagtatac ttaccaggaa aggaaagaga gaaggaggga |
| 66541 | gggaaggagg gagacatgga gggggcagag ggagagagag acaaagacac tgacaaagtc |
| 66601 | tggaaaaaga cacaaattca gatttaagat cccaaaatat gctacatttc tgcccccttc |
| 66661 | aaaatattaa actttgctaa aatacactgg aaattttttt tattgtttat tttttaaatc |
| 66721 | aactagcaga ccaattagcc aagatgatat tttcatcctt ggtactcttt caggtgagct |
| 66781 | ctgtgggcta ccaccagaaa gtcctagcca accaagctct cactgggtca ctctgtgcta |
| 66841 | gctagactac atagcctcct aaaaaagcac Rcagccctga tgctggtcct gccaaagtgt |
| 66901 | ggtttgctgt catgtcctac aggctacccg tttcccagtg atttatcata ataaaccaaa |
| 66961 | tgggaatgaa atacccagaa atatgccata aatctactag tggtgaattt tcaatattct |
| 67021 | gcacaccatg gtgatttgtt aattcagttg gatctctatt aaWcctctct aaaatgtcat |
| 67081 | taaRccatct gttttgactt gttaatcaaa caacttttaa aaatgtattt agagtatcag |
| 67141 | taacccttga attaatgcat ccatcagtaa taatcccctc tccaacttta gatttatttt |
| 67201 | taagctctag cttttttccc caccattcca acaaatagcc tgacttccaa aagtaagaat |
| 67261 | caatcacttc agttgtagaa atgtccagag ggtgccctac tttcgaaaca ggaagtaact |
| 67321 | tgatccagca gtcccacttc ttggtatata tccaaaagaa ctgaaatctg gatcttaaag |
| 67381 | agatattttc actgcagcat atttgtaata gctaagatac agaggcaacc taaatgtcca |
| 67441 | tcaacagatg aatggacaaa ggaaatgtga tatataYata cacacacaca cacacacaca |
| 67501 | cacacacaca cacacacaca cacacacaca cacacatata caatgaaata ttattcagcc |
| 67561 | ctaaaaaagg aaggaaatcc tgtcatttac aacaagttgg atggaactgg aggacatcat |
| 67621 | gctaagtgga ataaaccaga cacagaagga caaatactgc atgatctcac ttacatgtat |
| 67681 | aatccaaaat agtcaaactg atggaagcag aaagtagaat ggtagttgcc agaggctggg |
| 67741 | ggaagatggg aattggtaga tgtggtcaag ggttacaaag ttttagtctt gcaaaataag |
| 67801 | ttctggaaag ctgctgtata acataagtat acaatcaaca atactatatt gtatacttag |
| 67861 | aaatttggta aaagggtagc tgttatatta agtgtcctta ccatgaaagg aaaacaaaac |
| 67921 | aaaaaaaccc aaaaagatgg gaggaaactt ttggaggcaa tgaataagtt tatggcattg |
| 67981 | attgtaatgg tggttttagg gtgtatacct gtctccaaac atcttgcaga ataaacatat |
| 68041 | taaatctaaa gtttccttgg gaaagaagat gagaaaataa ttgtcaagag actgtacacc |
| 68101 | aataaaatag tcaaggagta caatgaaaac ttaggaatcc catttcttcc tttgactcac |
| 68161 | actgtttgat gatatcattc acatcggcat ttggaagtta ttgagacatt tcaacataag |
| 68221 | attgtcctta tgatctatga tttgggactc tcttataaga aagtccattc tataccaatt |
| 68281 | tgttttgttc cttggtaact agaccttctt taattggatc taagaattaa tattcctatc |
| 68341 | tcataaagaa gactaaaagg atccaagtag atggagtttc aaaggtggtc cccataaaag |
| 68401 | catttttacc ttaagcaaag atgtcccaat ttttcctgct tgcttttcgg atgctgttct |
| 68461 | aaaaaaaatg taccttattc cttctactaa aagaaaacct atcaattcat tgattcattg |
| 68521 | aacaaatatt tattgaacat gtagtatgta ctaaacactg ggtaagattc cagatatgca |
| 68581 | aagaggaata agtccctgcc cttaagaaaa ctcacagtgt ggtcaagcta acaattctag |
| 68641 | agcaactgtg tgataagggc tccaaaagca catgcaggaa cacagagaga ggcactctta |
| 68701 | gtcttggagg ggcttcctac agtgggcaat ttttgatgtg tcacaaaagg aggagctatt |
| 68761 | agggcacaag aagagcaagg gtgaagtgca gaaaggagtg aatagatggg gaggggaccc |
| 68821 | aggcaaagag aataacatca gcaaaggtgc aacttcatgg gaacacagta catttggaag |
| 68881 | cctgcccacc atttgttaca gcagaatgca tgtgtgggtg gagatgaggc tgcagagatg |
| 68941 | ggctggagag gattatgaga gatggatgac tagcaaagga agttggtcat gtccttatgt |
| 69001 | ggtgggggac catggaaaga tttttattta taagacgtgg tttattcttc attctgtggc |
| 69061 | aaaaagcaga tactatagtc tgaatgtttg tgtccctccc cacattcata tgcgatgaaa |
| 69121 | cctcatcacc aatgtgatgg tattaggagg tggggccttt gggagataat taggtcatga |
| 69181 | gagcacagcc cttataaggg gattagtggc ttataaagga ggcccaagag agctgccttg |
| 69241 | ccccttccac catgtgagga caaagccaga ggcactatct aYgacccagg aaatgagccc |
| 69301 | tcacaaaata ccaagtctac cgtgccttga ccttggacat tctagtctcc agaactgtga |
| 69361 | gaaataaatt tatgttgttt ataagccacc cagctcgtga tttatttatt tatttattta |
| 69421 | tttatttatt tatttattta tttagagaga gagttttgct ctgtcactcg ggctggattt |
| 69481 | cagtgccatg atctcagatc actgcaccct ctccttccca ggtgcaagtg attctagtgc |
| 69541 | cttagcctcc tgagttgctg ggattacagg tgcttgtgac cacatttggc tgattttttt |
| 69601 | gtatttttag tagagattag gtttcatcat gttgcccagg ctggtctgaa actcctgact |
| 69661 | tcaagtgatc tgcccacctc ggcctcccaa agtgctggga ttacaggcat gagccactgt |
| 69721 | gcctggctat gatattttgt tacagcagct tgaatggact aagacagaag gaaaaatagg |
| 69781 | cctttctgta gcgtgagctg aaagacagaa aatagccata ggtttctaaa gagagaaaaa |
| 69841 | gatatatgtt tgacaatatg tttcaattag tacttttttt gaaatgcagt aagttaccta |
| 69901 | taccttattc catttctaat ctatatgatg gcatttaaat attgactagg tttctgatac |
| 69961 | agtttttgga gtctgaatgt cactgcatgg atgaataaac caaacagatc ggattcatat |
| 70021 | gggtttgcca aatgacaaat ttttttccat gtaagattag aggaaaaact gatttcaccc |
| 70081 | aatttcctgc cttatttata ttagaactga agtcaatgct gttatccctt tgcatgttct |
| 70141 | tttgtattta aaatctgata tagaattgtg ctgttacaga agaaataatt taatgcattc |
| 70201 | tgccctctta tttctcatga gttttcattt aaaagaaaac aaaaaagaaa aaggacatgg |
| 70261 | acttttcttt atttctagac tttataaact ttttagttat caaataaaca catgtattaa |
| 70321 | ttaagtaggt aaaatattgc atatattaaa aggtacttgg agWctcatat gttgtcagtt |
| 70381 | tttcatgact caaaatttat gaaaaaccat ctaagactat cgtaatgaag ttcaattgta |
| 70441 | gatcttgaag tgacaaaaca tgaaccacat tgacttgagg tccagggctt aaaactgcct |
| 70501 | atttgagctc taggttaaaa gcatgtgtga aatgcaaagt ctgacatctg cacagctgac |
| 70561 | tattgtgaag tctcagtatc attgttcata agtaaagctt gtccctaagc cataccacaa |
| 70621 | gcattcccag aggatataga tttcatYtga tttaacatga ttgttgatat tggtagtgta |
| 70681 | tttcacgtga acttgtgaaa tcattgtcta tacagacaat tgagacacta aaactttttt |
| 70741 | ttaagtattt tttgttaggg gctagatctg aggtttttta ttcatttgtt cttttgttca |
| 70801 | ctcatttgtt agttcagtta tttactcaaa aatgtatatt gagcaattcc tatatggcag |
| 70861 | acactgacag atgctggatt cagtgatgag caaagcattc gtggtttcta tgcccaagga |
| 70921 | tcctatcccg tggagtgtag agtcaagcaa agaatgtcaa caacaaatga aaattttaat |
| 70981 | cttattaatg ttatatgcaa aaatgtaaca cctccaaaaa agacaattta tttcctatta |
| 71041 | aggaatgaag gcagtccact gaggaagatc aacatgtaag cacatgtaaa gcaaaataat |
| 71101 | caataggtaa tgtagttttt gaaaaagaag gggctttaag caggaagtcc tgggtttgga |
| 71161 | cttgggtttc aaatctagct atgtactctt gggccacata cccaagtctt gtaagtctca |
| 71221 | gtttcctcct ttttaaagtt agcctaaaac tacctcgctc atctaatggt gtgagaatta |
| 71281 | ggtaaacagc caggtagctc atcaaattgt aattattata attgttattt tttgagtcat |
| 71341 | gaaagttttc acactgaagg gaacatttca acttgacctg gaagagtaag tagtttatca |
| 71401 | ggcagaaaga atttgttagg gtattccaga gagaaggaac aaaatgtgca aattcaagta |
| 71461 | gtcagaatca aaatggtttc ttaaaggaat gacaaacaga tcagtgcagt tggagcctaa |
| 71521 | agttatctaa ataatccgtt agaccttttc tttaactgat aaaataaagc aagcattcca |
| 71581 | aaaatcaatt taaatccatc ttcctcctta tacctctcca ttccccatac catggaaaca |
| 71641 | cagttttacc tcttttacaa tgagttgtca gcatgaagag acttggttca ttaagattag |
| 71701 | caaacttcac agcattgaac aaaagataaa gaaaagttta tagcaaatac gttccatgtg |
| 71761 | aaagcatctc cagcaggttc agaagatgca atgaaaagac taaaacttga tatagggata |
| 71821 | tttcagtggt gctYgtacca gcgtcctaac attttgttct accttagtaa aatctatccc |
| 71881 | ttattactca tcctatctgt agactctctt atggaacaag aagctttttt ttttttttcc |
| 71941 | ctaatcccgt ggggaagccc tctccttccc tgggtgattc ctgagcaacc aggaaaacag |
| 72001 | cagtcacagt caacacagct agtacttgca gaaaatctct cttctgtgat gctctgaatg |
| 72061 | actcacagtt ccagatggcc ttgtcagcct ttatccgcat tccactcttg gcaccattaa |
| 72121 | cctggtctaS tgtggttgca acaaataact tttcttgtta tgaggaccag tgttttgaaa |
| 72181 | aatttaaaat gttataaaat taaaatgttt atatttggaa attatttcca aatataaaag |
| 72241 | gagcctgtct tcattataga ttatttggaa aaaatgcaga ggggtgcaaa agaaaaatgg |
| 72301 | aaacaagcca tctttttttt acccataatt ctaccactca gagacaacta ctgtgaacat |
| 72361 | tttgatgtat ttatctccag tctttctaga aggtagatca ctgtaagaga tttctgaagc |
| 72421 | agaagtacta agtcagaagg aataaacatt tttatacctc ttaatgtgta ttgccagata |
| 72481 | gtttttgaga aatattacag caatttctac ttttagaagt actgcttgag agtgatgtta |
| 72541 | catggtattc tcactaacac atatacaatg ttataaatat gtccaaaaat aaaacttagc |
| 72601 | aactatgtta tctccttgtt tcaatttgaa cttattacaa gagtgatcaa tttttaaatt |
| 72661 | taaatgttat ttttagcaaa gttatagatg caaatagttt agagaactaa gtagtaccac |
| 72721 | aaggattaaa aaaagagatc tcctgcagtg catcccccac ccacaataca tacatgtatc |
| 72781 | ttccactccc cagagggtaa ccattttcaa atattttagc tgattcattt gctatactcc |
| 72841 | actctatctc taaatgatat aactttacag ttacttctta atagttcatt tttttgggca |
| 72901 | ttgcttattg atacttcata gcggaagata aggatttacc tttgcaacac ttcacacttc |
| 72961 | tcatcaccca tccttctaat atattatagt cttacttaga ttgatagtca ttgtgtttcc |
| 73021 | atgagcagaa ccaaagaaaa actataagca gctaaactat gtaacatacc ctgattacct |
| 73081 | ttttttcttt gttttccttc ttgaaaattc ttgtcttttg ttagtttctt tgttccctta |
| 73141 | gatgtccata tatacttatc actaattcaa tcccaaactt ttcccatatt acaaaaatct |
| 73201 | aagtatggct caaacacatc aagaatttta ttaaattcat cttcttgaag aaatctcatc |
| 73261 | cagaggcctc tttgacctgg attggttatt ctccatgtct cttgctcagc tgtcactttg |
| 73321 | ggatgttctt tccttattat cccaagggta atgtttttag agtttattcc cttgttttgg |
| 73381 | tgagcacgtt ttccagtagc ttcctgagtc agtatgcatc agagatacat tttttaaagg |
| 73441 | ctcgcacctc agaaataatc tttattccat gctcactctt gatcaatagt ttggYtgagt |
| 73501 | atagaattct tggttgagaa tcactttcct tcggaattta aaaggcatca cttcattctt |
| 73561 | ttctaggttt caaaacgaat cttcagaagt ctgatgccct tttgattttt gatccttcta |
| 73621 | tacgatacga taatgatcct tagaacaagt cacaagtctc ttttttaata ctttatattg |
| 73681 | tgctaaatgc tccaattaat gagttctgga aatttttctt caattttgtc ctcctgcagt |
| 73741 | tttctctttt ttctagaatt tctgatttta gatgttagat ttcctgaact ggtcctctga |
| 73801 | tttcccactt tttcttctgt attttctacc tttttgtatt tttgctccat ttccatagag |
| 73861 | atgtcctcaa tttacctttc aacccttcta ttgagttttt aacttattct atcatgtttt |
| 73921 | tactttccaa gagctctttt cctattctct gagttgtttt ttttttttcc atagcatctg |
| 73981 | cttttgtttc gtggaagcaa tgttaatatt tatgaataca atatcaagtg tatttcttct |
| 74041 | cctctctctg atgatacctt cttcctgcaa catctgtact tttttatttc tcttcttttt |
| 74101 | tttttttttt ttttttgaga tggagtctca ctctgtcacc caggctggag tgcagtggga |
| 74161 | ccatgttggc tcactgcaac ctccatctcc tgggttcaag caattctcct gcctcagcct |
| 74221 | cctgagtagc tgggattaca gttgcctact accacgccca gctaattttt ttatattttt |
| 74281 | agtagagacg gggtttcacc atattggcca ggctggtctc gaactcctga cctaaggtaa |
| 74341 | tccccccgcc ttggcctccc gaagtgttgg gattacaggc gtgagccacc gcgcccggcc |
| 74401 | aacatctgta ctttgaagtt ggtctttttc catttgtttt ggtttctttc acttactttt |
| 74461 | tctaagtatt tgataaatct tatctgtcta ctcatattta aggctagagt actaaaaatc |
| 74521 | agattggaag ctctgtttgc aYaggtaaga tttgtcaact acatccactt tttgatgatc |
| 74581 | caactgaaca gtttcattca gaaatacctt gcaccattat ctttgggttt tttctcttgg |
| 74641 | gttggtgaat tctccaaagg ttatatcaat ctcctgtcca gaaggcttaa gcatagttgg |
| 74701 | aagctgacta ggggaagaag gttgggactt tcaaccttcg gaattcattc aacaaatatt |
| 74761 | tgagcatcta tatttggcag cctctattct agccaccgga gacacagtgg taaacagaat |
| 74821 | ttaaaaagcc ttgtgttatc atgaacacaa atatataagt gtttatttaa tccctctgtt |
| 74881 | ttcagaatgg cactcagtct tcaacctgcc taggcccccc ttgtctaggg acggttttac |
| 74941 | attcatcagg aagtataccc cttgtatact tgtttttcag cttccaaaat tttgttgctg |
| 75001 | ttatctccct tcccactttc tttgtccttg tcatttatgc ctataaacat ccattgatgg |
| 75061 | ttttctgaag gcagaggagt taaatggata tgttaattac tatagatgta atcagaggtc |
| 75121 | ttcatgttgt ttttttgttt tctgttttac tattctaact tggcatttat agatcttctt |
| 75181 | tgtgagtagt ctgaatttgt ctttaatgat ttaacattga aacagtattt tccttactta |
| 75241 | tttgcataag ctctttcttt gttagtgtat tagccattgR cctgcaatat cttttaggat |
| 75301 | gaccgattct gaagcctgct gactgagttg cttgtggttc ctgtcattta gaatgtatgc |
| 75361 | aggtcatgaa tcgcttagat ggaacagatg gaatgaaact tgctctaaat attttaccaa |
| 75421 | aataaataca tttaaataat gtaaagaaaa atcttcaatc actaggcttt gaacttataa |
| 75481 | cagaaaggta tctgctttta actatattaa ttcaacaaac tttattgaac agttggtaag |
| 75541 | tatgaagcat catctctctg agaacaaaat accaaggaac tgagtcccat cattacagga |
| 75601 | ttctcaggag taaatgggtc aatacatagt tataaaatga tgagggctga tctatagcag |
| 75661 | aagtacaaag ctgagaacaa ctcacttgac ctgctaagct ggggaattca ggaagggttc |
| 75721 | agatctaaag agcagacatt tgaggtggtt cttgaaatat gagcacaatg atggttttac |
| 75781 | caggctgcaa aggtgacaaa gtgcattcca ggctgataaa agagggcatg caaaatatgg |
| 75841 | caggtacaaa aatgtgtatt taaggaagaa tgagaagttt ctggaagttg ctgaagatac |
| 75901 | agaaaataaa gtggggggcg gggggggcgg gtggttggag atgaggagga gatggtaggc |
| 75961 | ttgattcaaa ttatggaaat gtatttataa tgcaccaact tgtaaagaca ctgttgttcc |
| 76021 | ctacccagca gccactagaa tttgttcagg tgtcaggcgg ttggtgcttc aagggagatc |
| 76081 | aggcccctcc caggaccagg atgaaccatg attacttccc agccaatttc atagcatttc |
| 76141 | tcttgccaat gaatggctta gatataagca tatgatgaat ttgtttctaa tacagatctg |
| 76201 | agaaagaaat ctgttaagga acttctggta aagtttttat ctttcttaaa aagtgacaga |
| 76261 | agaaaaggaa gttgtataaa tatacttaga gctgctgtag tcatcttgag accataagag |
| 76321 | gataaaactg agatcaaaaa ctactgtgct gaggatgggt gactagaaag aaggaaagaa |
| 76381 | tctggattct tgatgcatgt ttgtactact agaatcattc tatcactacc ttcttattat |
| 76441 | ataattcaat aaatctctta ttatttaagc tattttaaat gaagccttct gttatatgCa |
| 76501 | gcctaaacaa atatactgat tcaagctccc agcaaattct gtcacacata attcaaggaa |
| 76561 | cgtttgttaa taacttcata ctattatatg gggaataaat gattagtcaa gaaagacttt |
| 76621 | ctgttttgaa aaattctgga aagatgataa tacagtatgt agatttattg tcatggctaa |
| 76681 | tatacatcta catataacat gaattacctt ggagtagtaa taaaagggca gagtaataac |
| 76741 | ttgtgcagag ccctagacta gaaaagttga aaatataaaa tggaggaaaa agtgagttat |
| 76801 | gagattcagt aatatgaaag tataagaaat aaaagaatcc aaaagccata gaaggttgaa |
| 76861 | actgaaaaga attttagaaa gcgtctaggc caggtgtggt ggctcacacc tgtaatccta |
| 76921 | gcactttggg aggccgaggc aggcagattg cttgaggcca ggagttccag accagcctgg |
| 76981 | ccaacatggt gaaacctcat ctctactaaa atacgaaaac ataactaggc gtggtggtgc |
| 77041 | atgcctgtaa tcccagctac ttaggtggct gaggtggaag gattgcttca acctagggag |
| 77101 | gcagaggttg tagtgagcca agatcgtacc acttcattcc agcctgggtg acagagtgag |
| 77161 | acactgtttc aaaaaataaa aagagagtct agcctaaacc ccttaattat acagattgaa |
| 77221 | aaccagtcct ggagaaattg gaagtcctca aagagtttac cattaatggt ggaaccagga |
| 77281 | cttccacaca atgttctgac ttcctttaga gcacttttta ttatatcata tagttcctta |
| 77341 | cttctcacta ttaatagaga ttattttact aagagtaagt ataataagag ctgatcagaa |
| 77401 | gacctgagag acataatgga gtttcaattc taatttgctg ttggctcctt gaaatgcttt |
| 77461 | gttgagaagg agtaaaaagc ttacaaaaca cacacacaca cacacacaca cacacacaca |
| 77521 | cacacacaca cacacaattt cttaattatg tctaccatgg gcatgggatg gtaggaatgc |
| 77581 | catgtaacta gtttccatgg atggaaacca cctaatggga aatagagaaa aagaaaaaga |
| 77641 | aaagaaattg tgtgtaaatt ttcagctcat tttggcttag attacatgat gaaacttatt |
| 77701 | tctttgaaaa actcatttga ttttgaatta gattatatgg aacttctcct ttgtcaattt |
| 77761 | taatttcttg tcttgtttta gtttgctttt ctgagcagta gatatcctcc caattggaga |
| 77821 | ttccgcaaaa ttagtactta gtcatcttgg ttcctttcaa gtagaaaagt ttcttttaaa |
| 77881 | tacttcaaat cattggtatg cacatttgtt gcaaaaaaaa acctcattct ggaaaatgtt |
| 77941 | gcttatttta ttatccaaaa aataggtatt tattaatata ctgcatttct tttaaataaa |
| 78001 | ataaattaca gattttcttc cagcatccac ttatttggaa ttgggtacat atgaagaaaa |
| 78061 | gacttcttat aatatggaat tatttcaaaa gctattatta cttcttataa atataaagca |
| 78121 | tacaagaaat tctcaccatt ttctaagatt tacattagaa agatataagt attgtctttt |
| 78181 | agaatactgt ttttattttt ctaaatattc cttataactt acttattaat tttatccttt |
| 78241 | ctttgttata aatatccatt ttttttaaag acccatacta tcttttccta tgcaatttaa |
| 78301 | cctaacatat tttctgtaaa tgaggttatt tagttatcca tttttctacc tcctgttgct |
| 78351 | tggcagtcct caaaaccatt gcagatcttt ctagagagca gtttttaaga acagtgcttt |
| 78421 | acttttcaac tcttgatcaa cctgatgaga attctttgaa taaaccagaa ggaaggctaa |
| 78481 | agagacaaaa gaatactata gtttccttaa agttaatgag ccagaatcct ttggcccact |
| 78541 | ttggcattga acaatgtgaa taaaatggcc actctcacat cagtaattta cattttatac |
| 78601 | agtgtgtact ttacactgat atttacaaag aggaaaaaca acagtttttt acatgtaaga |
| 78661 | ggagctcctt ggcagataaa agaaaaactt actcccatta gtacaacact ggagtttaaa |
| 78721 | tctagagaac aagtacttca gaacctagta gaaaaagtcc ctgatttgta ctttgtatga |
| 78781 | acaattaggt ttgagaatat tgctatataa tcacagaaaa tactggctat tatactacgt |
| 78841 | gaagtcttca gtgacaaaga gagaaacaga aaaaatagtt aatgccataa caatgaaaat |
| 78901 | gtggaatgtg aggaacgccc agtgaccgta aatatctacc ttaataagcc tgacaatttg |
| 78961 | tatacagttc atgcaattaa caagtgtgat aaatatgatt attactctca agttgaaatc |
| 79021 | aattttcttg gctaaaagca aagaattgtt attgagcaga tgggattact tttctttgcc |
| 79081 | aatttatctt cataaaactc aatcaatgcc ttgccttaaa aatgtcaaat aactcagatt |
| 79141 | tcaggataac acatacaaac taatctcttc tgcctgtctc tacctgatca ctccctactt |
| 79201 | caaaagctcc aacaacatag tataactgac acaaatcaaa gcaaaaacta tttttctcac |
| 79261 | tgctacatgg tcttatcaat aatccagtat ttcaatatct aataaaagat accacaattc |
| 79321 | aaagcagtaa tgtgccccaa gctaccgatc cacaacatat atattgatat ctaaaaatat |
| 79381 | atggatataa cttgttatct actaaataag tttctttgca gtgtggtgtt aatagccaga |
| 79441 | attttaaata cataaaaact tgggttcaaa tcctaactct gccacttaaa tgctatatga |
| 79501 | caattggtca agttacttaa cctctctctg gtctcaagtt tcttgtctgt aaagctgtaa |
| 79561 | tagcttttag agtgctcttc tgaatattaa aaaggcatta tactcttagc ataatgtcta |
| 79621 | catagagtaa atatttaaca acaaaataac aaatattagg tgtgttattg gagaattgga |
| 79681 | gttttaaata aaggagttgt ccctaaggta cattcaattt aactatgata tttaaaactt |
| 79741 | caatttttca cattattttt ccaggaaaat atttcactct tatttctcca taatttgttt |
| 79801 | tgaattgtaa aagagatttg caagcggctg aagcagttac actatactgt atatctgcag |
| 79861 | gatgggcaac tgattttttt tttcttcttt tttttttttt tttgagacag agtctcactc |
| 79921 | tgttgcccag actggagtgc agtggtgcga tctcgctgca acctctgcct ctcaggttct |
| 79981 | agggattctc tgcatcagcc ccctgagtag ctgggactgc aggcacacgc caccacgcct |
| 80041 | ggctaatttt tgtattgatt ttcaagattt gtcaagagct cctaatattt tcttttctca |
| 80101 | aacacatctt aggactacca tgcatatcag tcagtcagct actctggagg tctgctgcaa |
| 80161 | ggatacacac cttccattcc cacccatcag atacttgtct cctttctccc ccactccaag |
| 80221 | acagcaacct tccagctcta gataaaaaga gctagtcttt cttgcataag aacaacaaag |
| 80281 | acaaaaacca ttttggtagt gtatactgca gattttaacc aaaacttctc tgaagacctc |
| 80341 | tgatttccta aaaaacctct tcatcccagt gcagttgctc tttcctaaat gagcaaccgc |
| 80401 | gcatctggtc aaatttggtg aatattctgt gctccagaaa caactgattc catccccttt |
| 80461 | aatctcttct tctcttatat ccatctccct ccttgttgtt tctctccatg tttacaacta |
| 80521 | gttcattgat ttaaaaacca atgtaataca caaggaagta tgcacgacaa tgtttagtgt |
| 80581 | agtatggttt gcaatattaa aaaaggccat cagtaagaaa atagttaaag gataaacttg |
| 80641 | atatacatac tatgctgcaa tttaagaaaa ctggtataga tttatacgta ctaaaataaa |
| 80701 | ataatctcca aaacatagta agtgcaaaga tcaatcacaR aataacatct gtagtatatc |
| 80761 | acttttgatt tttaaaaatt acccctaatt ctaaatgttc atatatataa atattcagtt |
| 80821 | aaaaattctg aaaagatact gctatggttt gaatgtttgt tccatccaaa actcaggctg |
| 80881 | aagtttaatt gccattgtaa cagtattaag aggtaggact cttttttttt tttttttggt |
| 80941 | gacagggtct cacttcaaca cccaggttag agtgcagtgg catgatcata gctcactgtg |
| 81001 | gcctcagctt cccaagctca ggtgattctc ccacctcagc ttccagagta gctggaacta |
| 81061 | caggcacatg ccaccatgcc cagctatttt gttgtatttt tttgtattga tagggtttct |
| 81121 | tcatgttgct caggcaggtt ggtctcgaac tccttacctc aagtgatcca cccacctcag |
| 81181 | cctcccaaag tgctgagatt acaggtgtga gccaccacat ccagcaagag ttgggacttc |
| 81241 | taagaggtgt taggtcatgt gagctttgcc ctgatgtaag ggttaatcct gttatcactg |
| 81301 | gagcaggttt gttataaaag agtgagacgt ttggtcccct ttcctctctc tctcccaatc |
| 81361 | tgtctatctc tctccctttt gctttctgcc atgtggtgat gcaacaaaag gacccttaac |
| 81421 | agatgccagc accttgattt tagatttccc agcctccaga actgtgagcc aataaatttc |
| 81481 | agctcatgat aaattaccta gtctcaagta ttctgttaga gcagcacaaa atggattaag |
| 81541 | gcagatatac accatattgt atccatcact aagtggatac tttcaagaaa gacagcaaga |
| 81601 | ctaaggagca tgaaaaagga ctttctcata ttctgaattt taaaaatatt ttatgacatg |
| 81661 | aatatattta tctattacct atgtaataaa actagacatt aagagaaaaa tctcatggta |
| 81721 | gaaatcttag gaaatagaga aaaattagag aaaattaaga aaaagcatgc ataatttcac |
| 81781 | aaaccaatgg tagccactgt aaaaaaatgt tatttcattc taatacattt ctacacacat |
| 81841 | atgcacacag tgcatttttg aaaacataac tgagtctttt ctaattgacc tcttactcta |
| 81901 | caaaatttga aatagccaaa acaaattttt gattcctata aagagatagc actagagctg |
| 81961 | gaagagatgg ctcatgcctg taatccctac actttgcatg actgaggcca gagaattgct |
| 82021 | tgaggccagg agtttgagac cagcctgggc aacatagcag gacccatctc tacaaaatta |
| 82081 | ataataataa taataataat aataagctgg gcataatggt gtgtgcctat tgtccttgct |
| 82141 | acccagaggc tgaggcagga ggactggttg agccctggaa gttcaaggct gcagtaagct |
| 82201 | atgattgcat tacactctac ccagtgtcaa ggaaaggaaa ggaaaggagg ggagaggagg |
| 82261 | ggagaggaga ggagaggaga ggagaggaga ggagaagggg aatgggcagg ggcaggggca |
| 82321 | aggcaaggaa aggggcaggg gcagaggcaa gggaagggga aagggcacta ggacctatga |
| 82381 | gccaacatag gctatggctt ccactagtac aacttaaaac tcagctgaag acatgacaca |
| 82441 | tagaaaaggg ttgctgtgtc tgtacacaca cacgtttccc atgttcccaa tcccagtctg |
| 82501 | tggtcagtga aagtgaagat tgcctcagag cctgcattat ctcaagctac tgacctaatt |
| 82561 | gctaatgctt tactctggYc tatttcctgc Mtgacctcag aatgtgtcag tgtaaattcc |
| 82621 | atagtaagac agtagttgca cattttggag tgtggattct gctttgaaac acaaataagt |
| 82681 | gtgatgaacg tgaccctggc actctgtagc cctcagtttc ctcaacgcta aattgatcac |
| 82741 | tacagttcct tctagtgtaa acagtcccac gaagaatcgt gctgaatgga tgactacata |
| 82801 | atggcctgct cctagagttc ttaaatcaat ccttaaaact ggctgcaaaa tgacttcact |
| 82861 | cctttaacag tgtgggactt gtctctgttc tccagaatgc tcaccagatg gtgaagaagt |
| 82921 | atctctctac ctaccaacat cgtcatattg cttcttgcca ctcgctactc aaaaaYtgtg |
| 82981 | aaaaacctgt gggagcaagt atcagctgaa aagggtggct ttctccttag gcaaaagctR |
| 83041 | cagggcaaaa cgggggcccc aaacttctat cacgtcaaag acatgaaaca gatgatccct |
| 83101 | agttttgatc ttctttgaag tttattaaaa acaagcttga atctgtacca atcctgacag |
| 83161 | gtgtgaatgt aagactcaca aatgagaaat gctaagcttt cctaatgttc aatttaatct |
| 83221 | tatagaccat gaaacatgtt ataattaaag atctgaacta ttaatatgaa ggagatagca |
| 83281 | tatattctta ccccaaatat gactaactag gtaaaatctt caaaatataa tcatggcctg |
| 83341 | attatcagcc attgtggagg agaattttaa aaagttcttc attgacatcc caagtttatg |
| 83401 | ttttagacac ttctaatcaa acatgaatag attaacttca agtaaaaatt aatagtcaca |
| 83461 | ttattgatta tcatgattgg aaatgtattt gaccagaaca atgaggaaag agcatagagt |
| 83521 | cctgctatct tcaagtatga atttacactt ttaactgatg tcaagtcatc catagtatga |
| 83581 | aaactcattt atttttattt gaaaaattta aaaccatcaa aatggatagt gtgggagcca |
| 83641 | aataatatat aagcaatatt ttatgagaac tacaaaacta ttaagagcag taactagaat |
| 83701 | tcggttactg ccgttcctta tttcagaaga gaaagtcaaa tgttttctga tatttaagaa |
| 83761 | gtaataactt cctgttgtto agttatgctt tctataatta tatatgataa gcaacagaaa |
| 83821 | aataggtctg gttttggctg taatgtgtat attcaattca atattaattt ggactttaaa |
| 83881 | tcattttgct atcaaatatt ttgaacctca tgaccaggaa gtaatcaggt aaaaaaaaaa |
| 83941 | aagaaaaaaa gtgggtttgt tacctgacct atctcttaag ttcaaacttt ctataggtat |
| 84001 | ttttctctac ctttgtccat atggttatac aattatcact agatgcttga attactaaat |
| 84061 | ggttttgtat atcttttcct caaatttata tttgtcctat tattggctta gtctctcaaa |
| 84121 | gtattacttt tatgatattg ctataatggt cctctatagt ataaagttga gcccacacca |
| 84181 | tttatcctgc cacttaacaa tgtccttaat gtagattata tggtttggcc atgccctaac |
| 84241 | caaaatctca tcttgaattg taatccccat aatccccatg tgttgcggga gggactgggt |
| 84301 | gaagataatt gaatcatagg gcagtttccc ccatgctgtt ctcatgataa tgaatgagtt |
| 84361 | tcacaagatc tgatggttat ataagcttct ggaattttcc ctgctggtgc ttactctctc |
| 84421 | ctgctgccct gtaaagaggt gcattctgcc atgattataa gtttcctgag gtctccccag |
| 84481 | ccatgcagaa ctgtgagtca attaaacctc ttttctttat aaattaccca gtctagggta |
| 84541 | tttcttcaca gcagcatgag aatggcctaa tacagtagta tatcacactt attaaagagt |
| 84601 | tgatagtaaa atagcactct cagtacaata agaatagaat agttcctttt catggtaatg |
| 84661 | aatagctttt cttcgacatc tataattaac aatgaaatca gaaacaaaaa gaagctgtct |
| 84721 | ccactattac tgaatgttgc tcagaaaatt ctaacgtaag caaccaaaca agaaaaccaa |
| 84781 | atatgctgta caaatatttt taagagagaa ggaaatcatt ctattttaag ataaaaattc |
| 84841 | tgtaactcaa aattcaggag aatcaactga aaggtataaa aaaggtagac cattatgatt |
| 84901 | tataagaaag gtcaaacatt ataaagcatg tatccacaac aggttttttt ctatatacca |
| 84961 | gttagaaaat agataaatga tcctattcat aatagtaata aaacatatca aatatgtata |
| 85021 | aataaacaac gaaaaatgta aaactcttgt ataaatatta ttgcaaaaca tcactgaagg |
| 85081 | acatttttaa aaacttgaag aatatatcta gtttcctaca tgggaagagt caatattata |
| 85141 | aagatatcaa gtcttagatt aatctatgag ttaaatgcag ttacttttgc cctaaaatgt |
| 85201 | taaaaggctc ttaattatgc aattcgaaat gtgatgtcta ctttcttctt gaaagcattt |
| 85261 | ttcctggaac cttctgatga ctctaactta ggctgcctgc tttctaggcc tactactaca |
| 85321 | cagttgtcat actgggattt attttcataa acatctgggt tggttccaat gctctttgta |
| 85381 | tcctaggttt tctgtatagg attcccgttg ctttgttttg cttgaatata tcttcgagta |
| 85441 | atacttcctt ttaattagga agaatctgta ggaggtaaag ttgtaaactt gagtccttcc |
| 85501 | atgtttaaat acatctttag tttgtcttaa cacttgatag tttgactatc aattttgggt |
| 85561 | tctagttctt cttcctgaga actttgaaac cttgcctagt ctgcattcac ttctaatctt |
| 85621 | gctgatgaga agtttgatac ctttgaaaga tccttttttt ttgttctctg gaaggattaa |
| 85681 | tttcttatac ttagaattcc aaaattttca tcagcatgtg tatacacatc tagatatgta |
| 85741 | tccttttttc cttgttcggt gttggatgaa acttttcaat ctgaggactt gaattactct |
| 85801 | tcaggtcaag gatttttttt ctgttatttt cccacatcct ttcccattcc tccacttcta |
| 85861 | ccacattctt catatctttt ttacttttcc atcRtatttt atatccttgg ctttttgctc |
| 85921 | tatattctaa gatatttgct taattcaata ttcaagaatg aatattactt gaattgcatt |
| 85981 | ttatccaatc tttatatata catatatatt cagtattctN ctatttatgg aaatcagatc |
| 86041 | atacttaatg gttatattct ccccctgaat atctctgaaa atttaatatt gacaaatttt |
| 86101 | tcccacactt ttcattgtct ctgtttcttc tggaaagcag aagataaatt gtctcaggaa |
| 86161 | aataaatttg tcctgtttat ttattagttc ctttttgttt aggctactgg tttttttgtt |
| 86221 | tggtttggtt ttttgtcttt tttcaaatgt ctggtgatcc tttcttgact gggaatattt |
| 86281 | gtaatttaag aactaggagt ttcttaagta gttggcaggg atatcataag gagctctgta |
| 86341 | gtacttataa actgcttata agcaacttaa tgcaatttca atcaaaatct taatgggttt |
| 86401 | ttttgggggg gacttgacaa gtagattgtt tgtataattt agcacaacga tgtgagaata |
| 86461 | acccaggaaa ttctaaaaaa taagattaag agggaatatg ttctcccaga tattaaaatg |
| 86521 | tatttcttaa aatccataat aattataaca tggggctttt tgtctagaaa taggatgtaa |
| 86581 | agattaaaaa aggaactaac cataaacagt agaagtggca tttcaagcca ataaggaaat |
| 86641 | aatagattat gtaataccta ggattctgac aatttagaaa atctaggaaa agaacatcag |
| 86701 | agccctgaaa taaagtaaaa taaattccat aattattaga gatataaata tatgatgtaa |
| 86761 | aatgattaaa tactagaaga aaatattggt gttggaaaag aattgctaag gatttataaa |
| 86821 | atatatttat atagagacta tataaacaat ataaacatac aggctacata gggactattt |
| 86881 | aaacttctat ttattaaaat agagtatctg taaaagtaaa aatagaaatg aataaatggc |
| 86941 | aaaaaaNtgt aaaaaacaaa cataaatgaa aagttaatat tcttaatctc taaagaacac |
| 87001 | tctaaaatca ataagaggcc aggggtggtg gctcacacct gtaatcccag cacttcgaga |
| 87061 | ggcagaggca gtcagatcac ttgagtccag gagtttgaga ccagcctggg caacaaagtg |
| 87121 | agacacctgt ctcctaaaaa ataaataaaa ataaaatcaa caagaaaaaa caaatcagcc |
| 87181 | cacgaataaa taataaaata ataaaaagca aatatgtata attgccatta gacacacaaa |
| 87241 | acagatttta atattcttga agctaaatta attcttatta aaacaaaata ctatgtgatt |
| 87301 | catcaaataa agagaacaat taaaatgata aaatatagca acggaaataa tgtagaaata |
| 87361 | gagactgctg atgaagtata tataaacttt ctgcagggca atatgacaat aatagcaaaa |
| 87421 | ataaatcttt aaaatattgg gcaattcttt agtttgacaa ttctaattct agaaatatat |
| 87481 | agtaagaaaa caacccagcc ttgctataaa gactctacac aaagatattc atcacaatag |
| 87541 | tgcttataat gtgaaatctg gggagagctt caaatattta ataatagaaa attgcttaag |
| 87601 | tgtagtatat tcatgtaagg aaatgccatt aaaatgttat atagagaatt gtatttaatg |
| 87661 | gtaagaaaag tgttcatgat gcaatatact gtgaaatttt aaaagcagat tataaaatat |
| 87721 | taccttcagt ataatctcat tgttgttagt aaaactcctt ctctctctac atacacactc |
| 87781 | tcacacacag agacagaaaa attatctctc aatggcataa ttattgacag ttcgttttct |
| 87841 | cccccttgct tattattatt ttgtaataga tatgctatag ccactcaata aatatttgtt |
| 87901 | actaatttca aaaagtatgt attattatat ccaggttgtt catggaattt atcatgcaaa |
| 87961 | caactaccct cttgagagag tcaaaagtgg acactataaa ttatgaattc atataaacta |
| 88021 | tatcaggaca atgggcataa actgagtgtc ccaggcacgg caaaatgtgc ggataccctg |
| 88081 | gagatgtatc ttgactttaa gaagtttaca atctttcttg gggatagaag attaaaaaac |
| 88141 | atgaaaccat tagaaataat ctcccacaat tagttttgta aaagcaagtg cttaattgta |
| 88201 | cttgacaaac attagatgtt actggaggtc taagaagtaa gagctgaata ctcgtcagaa |
| 88261 | aagctccctg gaggaagtgg ggactgagtg atacttttag aataggtaag aaaaagaata |
| 88321 | atgagaactt ttcaaatgga ggttggggtt gtagaaggaa aagaatcaag tattcagagg |
| 88381 | tagctgataa cgatgttgta tatataaaat caccctgact tttgcaaggg gtccgtctat |
| 88441 | acagaagagt aaaaagtaaa caatctatta acaatgtact atcactgcct ccactttaca |
| 88501 | caggagaaaa tcgaggcttt agaaggttac ataacctgcc aagggtcaca tagctaaaag |
| 88561 | tggagagctg agatttgaac ctaaggagtt ggtttcagag tctatgcttt ccaccctatg |
| 88621 | ctgtactgcc cactacagtc aagctctggc ttcttgtata aatctgatgt cactctttgt |
| 88681 | gggactgctt ataacatttc ctcacccaga atccatacct cctcctttct gctctgtcag |
| 88741 | agctctttgt ctattcacac acagttcaaa gaataccttc tccatcaaat ttttctaaca |
| 88801 | attccaatct tggaattctc tcctaccttg gagttgagaa caccactctc ctaaatttct |
| 88861 | gcctacttct cagacactac ttctcaatct ctttgttgga ttcttctttc tctatatttt |
| 88921 | tSttttttta gtctactcat tgcctgtgtg agtaccttca cccccaaaac tacaatacct |
| 88981 | acctaccatt gtgctgaaaa ccccagctta cattgctctt ccacattcca taaatttcta |
| 89041 | tctggatgtg cacagacacc tcaaacttca tatttccaat actggcattt accctatctg |
| 89101 | cagtttttac tatcttatgg acagcagatg ccagaaatct aatcactgaa accagaaacc |
| 89161 | catgtacaat tcttgagtcc tccctctctc aagcctaaaa tcctggaagt caccaagttc |
| 89221 | tgtctatttt actctataaa taactattag attcttcttt catctgaact cccttagttc |
| 89281 | aagccctctt tgtttctcat atgattattg tagtaggctc tgaactggtt ttcctggtac |
| 89341 | ctgtctttcc cctgcctact ccaattctac ctccacatca acatttctca aatttgaggg |
| 89401 | gtcatagact tccaaggaca tttccacaat ccctgagaag tatacagttt ccatatgaga |
| 89461 | aatcactgta gctaaaagtt aaatactttt cttcagttga ttatgtatag ctaaggcaat |
| 89521 | aattcaagca ttagtaaata caaagccaaa atgcaggcac attacgcaga aaatgcctgt |
| 89581 | tcttgggttt ttaaaaaata cgttaaaaaa ataaggccag atgtggtgac tcccacctgt |
| 89641 | catcccagca ctttgggagg cYgaggtggg atgattgctt gagaccagga gttcgagacc |
| 89701 | agccaagaaa acatagggag accttgtctc tacaaaaatt tttaaaaatt agccaggcac |
| 89761 | agtggtgcgt gcttatagtc ccagctactt gggaggctga ggcaggagga tcgtttgagc |
| 89821 | ctgggaggtc aaggctgcaa tgagccatga ttgccactgc tctccagcct gggtgacaga |
| 89881 | gcaagaccct atctcaataa ataaataaga ccacaaagga aaaaaattct tacagagaat |
| 89941 | tattgaagta taaattgtgt caaaattgaa gaatataata aaccctaaga ttaatagtcc |
| 90001 | tgtccttgag aaagatcatc aagtgatgcc cccatgcttt aaaaaatatt ccaagctttt |
| 90061 | tagcataata tctaacattt cttagcagtt cctaataggt tacttctctg tagttacctc |
| 90121 | tctcactcct tccccatcac ctatagtgcc acaatctgta acctgtttga ggctctctca |
| 90181 | cacctttcat actgcaatat ttgctcgaac tattgacccc caaccttact catctttcct |
| 90241 | ttctaactaa ctaacttctc ctcatcctca aagccccagc tcaagaattt tcatttctgg |
| 90301 | aagaatctct gattttcttc tcttctctag tcttgaaatg gtatgtgtga acccacagct |
| 90361 | tgttgtaatg tcattgtcct ttctgtttaa caccctcagt tgctggtcct tgaagtctgg |
| 90421 | gactgtctta atctacttta aagtcttcaa gccaagtaca attcctaggc atgcaataca |
| 90481 | tgtttgttaa atagatggat gacctttatt tatatttatc tttccctttt ctgctacaat |
| 90541 | tagttaactc attcatttct tcattcacta ataaccactt atctgttatt ttctgggaat |
| 90601 | acaaaaattc ataaaacatc atccttgcct ccaaaaaaca ctttattatt gtgccatcct |
| 90661 | tctctctctt tctctttctc tccttctcta attatctagt tgtgtaggta ttttcttcaa |
| 90721 | aggaataaca ggtactcaga ggaacactta tacactgttg atgggagtgt aaattagttc |
| 90781 | aaccattgtg gaagacagtg tggcaattcc tcaaagacct aaaaacagaa ctagcattca |
| 90841 | acccagcaat ctcatttctg ggtatatgcc caaaggaata taaatcattc tattataaag |
| 90901 | acacatgcat gtgtatgttc attacagcag cattaacaat agcaaagaca tagaatcaac |
| 90961 | ctaaatgccc aacagtagta gacaggataa agcaaatgtg gtacataaac accatggaat |
| 91021 | actatgaagc cataaaaaag aatgagatag tgtcctttcc aggaacactg atggagctgg |
| 91081 | aggccaatat ccttagcaaa ctaacacagg aactgcatgt tctcacttgt aagtgggagc |
| 91141 | taaatgatga caactcatgg acacatagag ggaaacaaca gacactgtga cctatcagag |
| 91201 | ggtggagggt gggaggagtg agaggatcag gaaaatcaac taatgggtat taggtgtaat |
| 91261 | atgtgggtga tgaaataatc tgtactacaa accccatgac acaagtttac ctatgtaaca |
| 91321 | aatctgtaca tgtacccctg aacttaaaag ttaaaaagaa ataaaaaata aacagaatag |
| 91381 | tagggattca ttacaatatc ttctctcctc ctcataattc ttaaacctgt gtagaagttc |
| 91441 | aatacatatt tgtggaatag ccttattacg cctttaggga atggagcaat gccttgccta |
| 91501 | ttaatctata cttctatcag agtttcctgc tgtatggaaa acctgagtgt tacacttcaa |
| 91561 | acaaatgagg caatttatac atttttgttg ttgttgttgt tgttgttttt ggcataaatg |
| 91621 | tgctaagtga gctggagttt cagaagagaa actgaatgtt atatgtgaat ccaggtcagt |
| 91681 | agcttggctc tgactacaat actgatttca ttcaagataa ggaaactaat tggtatcaat |
| 91741 | tttagttgac attatatgtt agttttataa tattcaggaa tttttgaagg attatttgat |
| 91801 | ttttattttt atagatgttc ttgctcttta acactaaata atgaggaaag ataatagggc |
| 91861 | taacatttat taagcattta tgttggacag gcactgcttt aagtattttt atatgtatca |
| 91921 | actcattgaa ttttcacaac tcttagatct atttgatagc tggggaaact gaggcacaga |
| 91981 | gaacctcaag taacttgccc aaggtaagcc agctagttag catggaccag tgattcaaaa |
| 92041 | acaggcagtc tgtactcctt acaactgtat tatgctgcct ataaaaggac cactagacta |
| 92101 | ataattatga aaccagattt ttaatcttaa attagccata taatagtaga gtgcatttaa |
| 92161 | ggatgtcatc tgagtttcat tgtccttgta ccccttacag cctgtgagaa ggaaccttta |
| 92221 | tatagtcttt cataaactgt aaagtgttat aaattcaaag atttcttata atatgttaaa |
| 92281 | attactttta agagttgaga agtccaaata aaagcagaga gatgagagga aacactagca |
| 92341 | ctaaatgttc agacacccac tgaccaYgtc aagctgcaca ttttgttgct aaacctaagg |
| 92401 | gagagattag atatgaggag agtgggaagc aagaaggtat gaagtctggg tatttaggga |
| 92461 | ctaatagaaa tgattttctc gattagttta agaacattag caattactta cgggataagc |
| 92521 | acagttaaag gaatttctgg aaggaataaa actataaaaa ttagtcaaat tgttaaaata |
| 92581 | tcagaactgt gtaaaagcat ataaagtttc atcatatctt tcttttgaaa aacaaacaaa |
| 92641 | cacttcatgt tctagccagt tgctaatagt gatcagttga tgaatacacc tcatgtttca |
| 92701 | cgcatataaa attttacacc atgtctagtc cataggcatt agtgtaggag tcagaataca |
| 92761 | acctgaattc tgttcgcagc tttgtgcctt gtcgataaag cagtggacat ctctccacag |
| 92821 | ggagatatgt ttcctcccgt gatatagagg atgttgattc acttgtctcc tctctatttc |
| 92881 | gaaaagatgc tctgaagatt agtgagacaa tggctgtaaa gCtctttgca ctgtttacaa |
| 92941 | cataaatcat acacagacat actggcatga gaaacgctat catcatctct tttatacatg |
| 93001 | agaacaacca taaggaaaac aaacctcaga gacagtcctg tattataaaa atgttgggta |
| 93061 | gagatgccaa aagttgtagg gtaggaaaca gcaacaacaa caacaagacg gaatccaaag |
| 93121 | aatatttgca ttctataata tattctacag agtYtcagac gggaccttcg ttgagtgctc |
| 93181 | ccaagtatta tcagtagcca tgtttttttt ttctttttct tttttttttt tttttttttg |
| 93241 | gtaaaaggga taaatagtga tgtcaggaga tgctgcaaaa tatattccag tgataagcat |
| 93301 | atggcaagca attaatggat gaattcgcct ctcttattgc catgttgtga caaatactct |
| 93361 | aacttatttg gcctttggct ccctaaaggg tgctcagaaa tgctgtttag aaagtacttt |
| 93421 | tcatgaagag ctacaacaac aacaaaaaca aatacaattc aaatatctac taaacatatg |
| 93481 | aaggagtact cactctcatt agtaatcaga gaaatgcaat gatcccatta tagagccctg |
| 93541 | tcatttgaaa aaaaagaaag aaaatccaag tgttttttat tagaacgtgg agcaacagga |
| 93601 | attctcatta ccttctcaac cactttgtca aatgctttct gcttatctgc tgaagttaaa |
| 93661 | gatatgcatg acccatgaat ccactcctag gtatattccc ccacaaaaat gtacgcttat |
| 93721 | gtgcatcaag atacacgcac ataagtgttc gaaatatctt tattcataat atttccaaac |
| 93781 | tggatatgtt caactgcata aagaaagaag attggacagt aaattgtggt tcctttatac |
| 93841 | aacagaatgc tatatatcat gatatatatt atgatgtgca atcctcagct acatacaaca |
| 93901 | ttgatgaatt ttacaactat aatattgcac aaaaggagca aaacacagga gtatctacaa |
| 93961 | gatgagtcca tttacataaa atgcaaaaca aactaaattg aactgttgtt ttggcatgca |
| 94021 | tacatacgtg gtaaatcata aagaaaagta ggaaaagtaa aagtaaagtc aagatagtgg |
| 94081 | tgacctatct ggagaggatg aaacaggaaa ttatagggtc tttctgaggg gtgatcatag |
| 94141 | taggtattta ctttagaaca atttgttaaa cttaaaattt aaattttaca tactttctta |
| 94201 | aattttgtta tatttcacaa tttaaaaatt tataaaatga taattttaca aagcctaaaa |
| 94261 | gatgaattat ttgggggctt ttacttctct ttcatctgta caactgctga tgaaaatcac |
| 94321 | caggtgtgta taacatgaag catctcctta aatgacatgc atttattggg aaaaaacaca |
| 94381 | aagtccaaat ggtaaaatgt tagcaaggta aaacactgaa gatttttctc cccaaaactg |
| 94441 | ccaaacaagt ctggtttaac tgaaattgac agcaaagaca acagagcaca cttaattgaa |
| 94501 | ttttaatctc agtgatctct aagccatgtt tgaaacataa acagacatgt cagagttctt |
| 94561 | tatgatgttg gacatgaatt cattttcata attcaaaggc atactttgct tatgaatctc |
| 94621 | tttaacaagc tttggctgaa tattcttttc ttccagatgt ttgctgttgt atacacacag |
| 94681 | cttccatctc tttgcagatg atttaaaaca tagttagaaa gagtacattt atttaacaaa |
| 94741 | agagtaagag tacagaattc aaatagatcg tctgtgggtg tatataatga tgagtacata |
| 94801 | agtctcatgc agtcctcata aagacagcat ctattaatct gaagaagttt ctcttactaa |
| 94861 | tttgcaaacc taaaagcatt ttacattact aagatattaa taaataggtc aattaatact |
| 94921 | tttgatagag tagagagact cctaaataat ggacatgaaa aatgattttg taaaagtaYg |
| 94981 | gtttcatttt aaatccctgc ttaagcaatt tgtaggaggc aagatagtgt ggcagttgag |
| 95041 | aggatggtct ctgaaatcag atctgaattt gaataccacc tctaccattt actaacctaa |
| 95101 | acaagtcata ttacttctct gtgtctcaat agctacttcc tgaggtgttc ttacaaaaac |
| 95161 | acaaataaat aatccatgta aagaactttg cacagtgcaa atacatagtg agcacgcatt |
| 95221 | gagttttatt tatcatcatc atcatcttgc tgtacttttg taaactcacc ttctcccgtc |
| 95281 | tttttctact ctaaggtgct cttccaagct actttcctcc tcagctatcg ttcccctgga |
| 95341 | tttccagtaa gagagggtga aatcatgcct gcttatcttc tttctctttt actgatactt |
| 95401 | cactgaagtg aaagtaaagg aataaaaaca caacacaccc acccacaaag gcacaaataa |
| 95461 | taaaggaaac aagatcagca aaggagagat ttctactgat tttttgaaat cagaaaatag |
| 95521 | ctgaagtgcc ttagcataat cgaggaaatg gttacggaga gtaaattcca agagaaagtg |
| 95581 | gactaatgtg ttctcccgaa tccagaaaga cttactgctt ggaggcagca gaattaagga |
| 95641 | aggcaggagt ttagcgaggg gctgaaagtg tattaaaatt atatggagcc tttggatcca |
| 95701 | aactaactac ttcccttacc tactggaagc ggagtctttc ttttttggag gaaatcggct |
| 95761 | agagagattt gaaactgatg atactaggca ctgtagcaaa ctaggagagg ctgcaggttg |
| 95821 | cgaaataggt gcttatttga aagcctatag agtttgcaat aagaaaccca acttttttat |
| 95881 | ttgtcatgct ccccagacat tagcagtcag ttatatgccc caggcgcaaa tctcgcaggc |
| 95941 | agaaagtggg gtacccccta atcatgacct tggggaaaca tttcaaagaa cacaccggct |
| 96001 | ttgattgaac aacctatgat gcaacagtgc aaccctacag ttgacgagcc ctatccatga |
| 96061 | tcacagaact tctgacattg tttcttgcca tcattgtcat tgttgctttg ttttgtgtgt |
| 96121 | gtgtgcatat gcacctgtat atgtcttgct tttaactaag aatgaacagc ctcttaagga |
| 96181 | aactctccat aatgaagaag agaaactaga agaagcaaac atcaactgaa gaaagaaatc |
| 96241 | tacaggaaag agagagacag aggaaaacct caagaggata ataatctgtg accttagata |
| 96301 | aaacacactt ctgacataag aacatatacc attaaaaagg agaggatgag gctgggcatg |
| 96361 | ttggctcatg cctgtaatcc cagcactttg ggaggctgag gtgggcggat catgaggtca |
| 96421 | agagatcgag atcaccctgg ccaacatggt gaaaccccat ctctactaaa aatacaaaaa |
| 96481 | ttagctgggt gtggtggcat gctcctgtag tcccagttac tcgggaggct gaggcaggag |
| 96541 | aattgcttga acctgggagg cagaggttgc agtgagccaa gattatgcca ctgcactcca |
| 96601 | gcctggtgac agagcaagac tccgtctcaa aaaaaaaaaa aaaaaaaaaa aaaaaaagga |
| 96661 | gagaatgaga gtgctaaatt atttttaaat catgaccaaa ataaacattt taatagtaag |
| 96721 | gttggaaaat aaaaatgtgt gtgaacaaat cttctagaag accaaaaaac aaaaagacaa |
| 96781 | aaaaggaaga aaacaattaa gaaattaaag gatatgatct aggagatcca actaagataa |
| 96841 | tgagaattca aggaagaaag aacagagaaa atagagggca taaaattatc aatgaataag |
| 96901 | agcaaacatt tcagaactga aggactcata tctctggagt gaaaaggtct attgcgtgcc |
| 96961 | cagggcaatg gataaaaatg cagaccaaga caattgatca tgaaatgtgg gaacttcagt |
| 97021 | aaagaaaaga aggtcacaaa aatttctaca cagaaaaaga tcaggatttc accaatagaa |
| 97081 | aatactggag tgatgcctac aaaatgttca aaaaaataca tttttaacat agaatccaat |
| 97141 | tgcagttaat ccatgcctga gacaaaaaaa ggtttaaaca aagggctgcc tctgaatagt |
| 97201 | atatagaaaa gatttctgaa cagaaaatct taaaaactag cttttatccc caacttggat |
| 97261 | actaaatata gagacaggca agaattaggg gtcttcttgt cattcttctg agactctgga |
| 97321 | gctcctgaga cttctttttc actaacacca ttgaatccgg gaatcaacaa gctgcacaaa |
| 97381 | gttataaaga ttgccagact tcatgtttct ggaaaaaaaa aagtactatt ttttcataca |
| 97441 | gctgagacct ctgaaaagat ggctcaatgg gcaagatgcc atgagaaatc aaagccaaat |
| 97501 | actcattctg aagtcagttt tgctcagact ttgtatactg ccactcaatt tttcaagtca |
| 97561 | tcagaaacat aactaagaat gagtgacttt ttgctatYaa ctaatgaccc accattcaac |
| 97621 | gatcttgtat gacaaagtag aggagtatct tcttattttt aggcttatta aattacaatg |
| 97681 | gaaaatattc attaacaatc tcataatgag gcattctaga tgcttattct tacctaaagg |
| 97741 | gaatggaaca aaaaataacc aaaaactaaa aagatgaaga caaaagcctt tcttggtatt |
| 97801 | atagtattct tttacaaagg atatggttta tcatagtaat tgattccctc tgttacacta |
| 97861 | gagatactat tatattaaat gcctaaaggt taaacttagg taacagtgga gctatagttc |
| 97921 | tttcagaaat aaaataatta aatagctatg tatgccaaga aaccttactt tgaacatttc |
| 97981 | tattgtacca tctcattatt gctggctgcc tgattagcaa gcagtaaact tgggtaatag |
| 98041 | ctttcctctg atatcacttc aagtctactc gagttctaga attacccaga ctaccaggac |
| 98101 | tttacttgca caatatgctg aaatgtttat aacattatgc tttcccctcc taattctcaa |
| 98161 | gaccattttg ataactggct aagactggat aatatcaaca aatatgaaat aattataaga |
| 98221 | atcattcttt cttaaaataa gaagagtttc cagaaacaga aactaaaaca gaattcaggc |
| 98281 | taagctgcta aataggctat ctcagggatt ctcagctcat tcttgatgag gatagattgc |
| 98341 | tttttatttc ccttaagtgt tccgtattta tttcacaaat attacttaac tgtgtaccat |
| 98401 | gtacaaagcc gtgtgctagt taagctggtt actgtggaag aaataaaggg agtgggagca |
| 98461 | tctagctttt ttcttcaaag gagatggggg aatctgtggg agttagtggt aggggcacct |
| 98521 | acagaggaaa tWgctgaaaa ggaggagctg gggaagatga ggatgaggaa ctaagttgtg |
| 98581 | aataaaggaa gggcagagca atggtttttt taataatatt ggaatgggat ttgtttttag |
| 98641 | cactgctccc ctttccctta acagtcccct tgagctacaa ataaatgtaa ctggcaaagt |
| 98701 | ggataataag acttcagaat cttctagcta accatgatca atgtgtcatg |

Following is a genomic nucleotide sequence of a GPX3 region (SEQ ID NO: 5).

| >5:150384801-150484800 | |
|---|---|
| 1 | ggcttatgtc tacaatccca gcactttggg aggccgaagc aggcagatca cttgaggtca |
| 61 | ggagtttgag accagcctag ccaacatggt gaaaccctgt ctctacaaaa aaatacaaaa |
| 121 | ttagccaggc gtggtggcac acaagaggct taggtggaag aatcgcttga acccaggagg |
| 181 | cagaggtttc agtgagccaa gattacccca ctgcactcca gcctgggtga Yagagtgagg |
| 241 | ccctgtctca aaaaaagagg aagaaaggaa gggagggaag gagggaggga ggaaggagca |
| 301 | catacttgtc tgaggatcag taataatacR tataataata acatctgact ttcattgaaa |
| 361 | acatattatg tgccaggtag tattctgagt gcttaaatct attgattcat tctgtcctca |
| 421 | caactacaag gtagtcactg ttactattct tttttataga acagaaaatt gaggcccaaa |
| 481 | gagatcaaat aacttaccct gtccctaatt agtaatggag ctaggactca gaccatggta |
| 541 | agctggattc agagcctttg tacttaattg caatgcctct tScatatgYg ctaggtaggg |
| 601 | aacaaggtga tagcagaatt taatagtcag gattgatgca gtattcctga actagtatag |
| 661 | aatgagctag attaaaaact cagcaattag aattcagtag cgaatcaaat cctgagataa |
| 721 | gtaggatttg gcttatcctg tggccaattt tcctcgtatt aaacatataa attcatgggc |
| 781 | cttcacaccc ataaaagaat aaattagtat tataattttt aaaatatgat taaaattagt |
| 841 | gctttctgaa gtactggtta ggtgtttctg tttgcaaaga gaatttatgg ttttgtaaga |
| 901 | ttagtttaat gttgatggtg gaatagacaa gagccaaaag acactagaag caaagagaaa |
| 961 | aatgaaaatt aaactatagc aatccagcag ttagttaata aggctgcagc tcagatattc |
| 1021 | ctgttcctca tccagtcctt actttttttc tccaatcacY gttgtYaccc tggtcagttt |
| 1081 | taattctgct aaataagctc tccccactga ccctgttgca actgtccttc tttgttcctt |
| 1141 | tttactattc ctcaagatta tttagtgttt ctgcccagtt tgcaaaatcc tatttgtata |
| 1201 | tagccatata tactttgttc attgtcatat tctgtttctg tttctactac tgcaattttt |
| 1261 | aaaaaatgtg gttttctttc ttgaaatgct cagggggata gactccattt Rgccttgata |
| 1321 | tttcttgtag gccatatgta tattatgcat gaaagtaaag attcaatctc ataatatgac |
| 1381 | aaaaagttat gagtcataat acattagcag atatatttac cagtatatag ttaattgtgc |
| 1441 | ttctttctag aagtctggca agacttccaa ccacatgcag aggcaccagg agaaccaata |
| 1501 | aacacttttg aggcatgttt cattaataga ccagggaact gagacagagg aaaggKgtga |
| 1561 | caactgtaat gctcttgaaa atatatttca tctgagccca gactttattt caaaaattcc |
| 1621 | aaaaatgtgg ctgttatggt aggaatttga actaYttgga tttatttagt ggcaatagaa |
| 1681 | gatatgccac aMaaaaaaag agcatagtga atgtgaaaaa gcctccttcc agaagtcaga |
| 1741 | cctcattatg caaaagagaa ccataagaga gaaaagccct taatatgtac taagtgtggg |
| 1801 | aRaaccttca gcagggaatc aaatctcatt atacacctga gaatccatac tcaagagaaa |
| 1861 | acctacgagt gtgttgaatg tagaaaagct ttctcccaca agtcacatct gttgaacgtt |
| 1921 | agagaattca caccagggag aaaccctatg agtgtggcga gtgtgggaaa gcctttttcc |
| 1981 | aaaacttaca cctcagcatt tacaggagaa ctcatactag aggaaaaccc tatgtgtgtg |
| 2041 | atgaatatgg gagagccttc Wgcctgaagt cacacctctt tgtacatcag agaatacaca |
| 2101 | caggagagaa aWcttatgtg tgaactaagt gtgagagagc tttcagtgaa gtRtcatgcc |
| 2161 | ttattagaca tcagaaaatt tacactagag agaaacctta tacatgtaat gagtgttaaa |
| 2221 | aagttttttc tcaaaagcct gacctcatac attacagaat tcacacaggg aaaaaaactc |
| 2281 | atgtaaatgc agtcaatgtg taaaaaaaag ccttcagaca tagatcagcc ctctgtcgac |
| 2341 | ataagaggac tcataaagaa aaacatccct gaacaagaaa taaatatggg aatatcttta |
| 2401 | actagaactt gtagtgatag aaagccttca gtcaaaagca aaccttatta tttatcaaga |
| 2461 | aatcttactg aggataagcc tgtttaatat atttataaaa gcgcgcagtt atgtttctat |
| 2521 | agaaaacata ttccagatgt gattcctaaa tatttcttga ggatattaca gaaaatacat |
| 2581 | ttttattaga gaataaatgt tcactgtgga ccacaaaggc ttttaaaata ttaataagtg |
| 2641 | aactagtcag aacaaactta gaaaacRata tgtggacaac ccacaagata tgaatgttac |
| 2701 | tatatgtgcc actacttatt gctgtaagtc ctggctgcac tacattttgc tatgcatcat |
| 2761 | ataaaataag ttatatgtgt tggaattgca tatgtgaatc taaaaatttg taaatataaa |
| 2821 | ataattacta tatatagaat gccatttact aagactttct acattaaata ttaccatttt |
| 2881 | tcccctaaag aaaatacaag ttataaccaa aaatacagtg ttcagtaaga gttctaaagt |
| 2941 | gtaaaataaa cttttttctg ttgctttctg gtatgtatga cttctgctgc ttcttaatga |
| 3001 | gaagtacata aaggtgttaa ttacataaaa tatataaata taaactagat atcatataag |
| 3061 | tctcagtagc aaaagaaaat agtcatggcc tcttacaaga ctcactgcaa tttaRaaagg |
| 3121 | aattcttggt gggtacaatt acccttttta catactgtta tggactgaat tgtaccttcc |
| 3181 | caccaRattc atatgttgaa gccctaatca ccaatacctc agaatatgac tgtatttgga |
| 3241 | catactgcct ctaaagagta attaaaataa aaccattagg atgtgtccta atctaatctg |
| 3301 | actggtgttc ttacaagacc tagagagtag gacacacgag gagacatcag gggcaggtgt |
| 3361 | acacagagaa atgaccatgt gaagtggcag caagagggct gctatctgct agccaagaag |
| 3421 | agaggcctcc aaggaaacca acccttttgc acattgatct cagacttcca gcctccagaa |
| 3481 | ttgtgagaaa ataaatttct gtcatttaag ccactcactc taatattttg taaggcagcc |
| 3541 | ctaggaaaca aatacaaaca ctactgctct ttttccctgg agtactttgt aagcttgagt |
| 3601 | atacaaagta taagtactac gtaagccaat aatatgatag agtgcttgct atactgcacg |
| 3661 | gcatctctat ttggcatttt ctaattcacc ttgtgacaga ttagtaagag agatattttc |
| 3721 | ataaggattt tacaattcac aaatattata aagacttttt gcagagagaa ttgttcattt |
| 3781 | ttaaaattcc atctaacagt aggggtaggt attctgtaaa tttccatgtg agtatccatg |
| 3841 | ccattaaaaa tcaacttgtt ccttatttat aaattaaaag tggggcctca ttcttctaaa |
| 3901 | gcaaattttg acagggagtt aagattacaa ggcagaaacc tacaagaggt gttaagagct |
| 3961 | cttattctgg atttagacag atgtagattc tgaatctgct ctgagcctca atagcaccta |
| 4021 | gtttgttgga tcattgcaag gattatacaa tatcatgtat gtatggtgct cataagatgt |
| 4081 | tagctatttt atattattta aggatataat tcocattcta gttatgaaga cagacaagga |
| 4141 | aacaactcta attaggatta tattagagag aactgtaggg agtcatgtgt atcattctgt |
| 4201 | ttcattctca caaccatgtc agtctttcag actcaggtgt gggcctggaa ccactgccct |
| 4261 | gaattgcccc tctacatcta ttctataatt gcttgttcag aagcaatacc attgctgata |
| 4321 | ccccaaactc cttatcttta tctcatctac tgatgagacc cattgcttgg agggtagtta |
| 4381 | catggaaagc cattcacaaa acaccttatt aactttgtgg gtgaaaaaaa agtgtatctt |
| 4441 | aattgtatta agccactgag atttagggat tgattattaa tagcaaatag caatcatttt |
| 4501 | ctagtagaaa gaaattggtc ctggaagtgt cctgctaatg taataaaaac ctaaaatatg |
| 4561 | gcataggcct agtagttgag cagtgagaaa actgatgtca gaagtggaaa gatgtttatc |
| 4621 | tatgttttgt actagccaaa catttggttt gggccaggcg tggtggctca cacctgtaat |
| 4681 | ttcagcactt tgggaggtca aggtaggcaa attgcttgag cccaggagtt caagaccagc |
| 4741 | ctagacaatg tgatgaaacc ctgtctctat aaaaaataca aaaattagcc agatgtggtc |
| 4801 | gtgtatacct atagtcccag ttactcaaga gcctgaggca ggaggatttc ttgagcccag |
| 4861 | gaggttgagg ctacagtgag ctgtgatttt gtcgctgtac tccagcctgg gtgacagagc |
| 4921 | aagaccctgt ctcaaacaaa acaaaacaaa acaaaacaaa acatttggtt tgattgtctc |
| 4981 | ctctacacat gcttactttg ttgacagctc cagaagataa ttagaaaaca gagtattagt |
| 5041 | aatatttgct gttttactgg ctgtatttgg aagctattgt aagaaaaaga taaactcaga |
| 5101 | gaaaattagt ggattataag caaaaatgaa agaaaataga gtcctgaaat ttgagacctc |
| 5161 | atggagttta aaaaatttga tttctagact tcaaatgtta ggagaataat ttaaatggtt |
| 5221 | ttgagtgtat caaagaccta ataaacattt gctagtgaaa taaagtgact cagaagaaag |
| 5281 | attatattaa gaaactgaaa ccttctcgtt caagcttgat aaccttacag tagtctgtta |
| 5341 | ttcacctacc ctagctccat gatggagaag ctcaatttta ggcagctaca gccaagaaga |
| 5401 | ctggggctcc catacccaac ctagctccca cttgtagagc agaggctctt gccaaggtaa |
| 5461 | gagaggctaa gagtattggg acctcaattg ccctcctcct cagcttttct ctatagagta |
| 5521 | gatgtttcat gccaggaaag gcaagccaaa aaaaagatca ttctcccagc tcccactaat |
| 5581 | aggatagagg ctccagactg gcaggagtac attaaaaaga Yctcaggcct tcatcttcct |
| 5641 | accaagatct cacttgcagg aaggagtatc cctgctaaga agcttccccg gattccactg |
| 5701 | gtagggcaga ggtgtcactt caggaaaatc cggcKttccc ctttgtcccc cgcttctgtg |
| 5761 | caagagtgta gatgttatat ccagggaaag aagcaagatg ggagggccag aagctcctca |
| 5821 | gctcagctSg ggggctgact gtatttaatc ataacatgaa gggttgcatg aattaaggca |
| 5881 | ttgttgaaaa tagagatctt agtggcaagc acttaagaga attctaatag tttcataaaa |
| 5941 | caagtagcaa caaatgaaac agcaagtcac ccggtttaac agagtgaaca agagaaagag |
| 6001 | ctaaaactgg aagtctgtgc atatgtctat gtttcacaca ttcaggacca accataacag |
| 6061 | gtactagctg aacttgagtg gaattgaaag aatttcccaa gccacacgct gatacattag |
| 6121 | caaagagtgg aagtcttact agctcaagga gtgtaagcac aacccctgac caaacactgg |
| 6181 | ctaaatgtta agatattttg acccaacggt gacttctagg aagccaggct tggggtaaaa |
| 6241 | gctacacact gtggggtggg gggcggcggg gggaaataga gtttacaaag ttggtgcaag |
| 6301 | caagccacta aacaaactaa caaaacagag ccacatcaat aatccctaaa ggggatgtgt |
| 6361 | cagaatccag agttgctaca atatattatt tcaaaaacta ttagagatgc aaaagaacaa |
| 6421 | aatagtataa cccttgtaaa ggaaaaacag catcaataaa aactgctttt gaggaggtct |
| 6481 | agatgttgga cctgtaaggc aaagatgcca aagaagctat tacaaatatg ttcaaaaaac |
| 6541 | taaagaaaac catgttcaaa gaatgaaagg aatgtatggt gacaatgact catcaaatgg |
| 6601 | agaatgttag tagaataaaa agtgtttaaa gactcaaata aaaattctgg agttgaaMag |
| 6661 | tagaatacct gaaatgaaga ttcactttag gtactcaaaa gtagatttga gctggcagaa |
| 6721 | gaatcagtaa gcttggggct cgatcattcg atattataca gtcttagaac agaaagaaaa |
| 6781 | aataatgaag aaaacctgag cagacttctg gtttctagtc tgacatgtaa agagcttgga |
| 6841 | agttgtcaat cctatcatca caaccagaaa aaagtctgaa aaactggaaa ttaacaattc |
| 6901 | ttagatcctt caaagaatca aggtcacagg acaaaccact gccccagaaa ctagagagac |
| 6961 | aggtagatgc tgagaatcac aggttcactg acagcggaag tccaccacta aagtcaatat |
| 7021 | attttaggaa cacttaaact acaattgatg aattgctaga gactagcaat aaaagtgcca |
| 7081 | gcattctagt tgatgaaaat gttgtgacca gaggcttaca tgaacctaat cctgtattcc |
| 7141 | tcttggagca attattcagt atttactaat acagtgtttg tggtggattt atttaacata |
| 7201 | agtactgtga ataataaaat caactgtata tgaaatcttg actatccatt tacatgtagg |
| 7261 | agaatcaaca aatggtattc tccacaaatg gaatcaaccc ctagaagttc ctgagaaatc |
| 7321 | cctgttgagt gggaaaggac ctagagccaa ggagacaagg ttggaggtga ctggtcctct |
| 7381 | tcctttgttc ttccactgaa attacttgag aagcatcatg acacatcctt atacacaggg |
| 7441 | tatggtggaa aaataattgg cagtcaaaaa gacttaagtg tatgaagact taagcatgat |
| 7501 | acacatggag aaggtgatgg tgtaaagaat gacaatggcg tctgagtaac attctgaagg |
| 7561 | aggagggaag ttcaaaaggt gggatggtgt gcctggatga gacacatgag ggagaaggat |
| 7621 | tttctatggt gcttctgtta gtttagcagg tagaatgccc ctgtgaaggg tacaggaagc |
| 7681 | atgtggcaga gtgggtgcct aagtctgggt ttagggagac tttagaaaca atttgaaacc |
| 7741 | cagggtctta ccctctgttc acctcataga acagtaaaat cccttggatc aggcaaactc |
| 7801 | tgatgaaaaa cccagactgc catctctctc ccagatggcc aattttcctt ctggtgctgg |
| 7861 | cgaatgatga agttgtcttc aggaaggcag aattctgctg Sgtgctctcc tcttctgagg |
| 7921 | agttatacca ccacacagcc tttgctccca gaaagagtca ccatcttgtg ttctaacatg |
| 7981 | gatcccttgt aagcccatac aaaccactcc atccatactt aactgctttc tccacaaagg |
| 8041 | caactgagtc taggagccaa tggactatct cactttattt tttaaaattt acccagggca |
| 8101 | ctttttcatg ttctttcttc tccctggaac ttactagcat tatggaacac aaggatgtgt |
| 8161 | ttgtggtcat cttctcttta caagataggt cacatagacc cataagtagg ctgtgtatac |
| 8221 | ttttcttaca aaggaggtgt cacttacctt gggaatacca gccagccaca tatgaaatcc |
| 8281 | cagacacagg agacccctaa gaaaaaggcg aggctgtctg taactgcctt agcaactaag |
| 8341 | ccaccagtgg attcaactct taaaattaca gcaccatgga aacacaaaat cacagaattt |
| 8401 | tggatttggg taacactaga gagctcacat aattaaatac cttaatagaa aattctcatt |
| 8461 | tactaattag aagtttttaa ccatgtagtg taaccatgta tacactattc tcaaagagaa |
| 8521 | tgggaatatc ttcttcagtt acttaatgaa aacatacaag gaatctgaaa ccctaacata |
| 8581 | ccagaatcca tgggacaaaa ggaaaattat ttgatcatgg gattcagaag catttactgg |
| 8641 | atcaaggttt agagatgctg aaagtagaga atcagtgtta ggttttggag ggaaggcaag |
| 8701 | ggttaaagga agacagagag agaaacagag caactcaaca gcaaacacag gtatattgca |
| 8761 | gacacctgtg gaagcgggag acccgcttaa tgccaaaacc caccaccact tacaagcagc |
| 8821 | agtacttaca ggtatggctg ggagaagtct ggatagtgtg gcttgctgcc cggcaggata |
| 8881 | tcaataagat gtacccatga tagggtggtt tgaccctttt tctggtggga tgtcatcatg |
| 8941 | gtgttccttg ggcctttgcc cagtaagaca ggatagggag gtttctttag ttgggccttt |
| 9001 | gcctggtagg gtataataag aatgtttttt ttattgggcc tttgtcttcc ttgtggtcag |
| 9061 | gtggccaggt aggatgtttc tcatggccca aacccccgtg gaatgtttca ctttgaccaa |
| 9121 | ggtctgcaaa atagcagggg acttacaaaa tggtgccgtt tgcactaaca caaaggtgaa |
| 9181 | tggcatgggg aatggtaggt caagaaagct gcttccttaa tttatccctt ctcttgaaat |
| 9241 | gtcttcatta ttaagacttt cattttaact gcctttggag ttaaaatata aaatatgaat |
| 9301 | tttaacacag aagtgtgaat gctggtattt ttgaaatcaa gttcttataa tcccaggcta |
| 9361 | gtgacagctt gcctgggcaa ctaagtaact taagttcata agtaaacact aactctgctg |
| 9421 | aaactaaata actcatttta gcttctgaac attttaagca tttctcattc tggataagaa |
| 9481 | agagaaatgc tgaagcagaa tttatttcat ctgctttaga tgaatttagg aatcccttgc |
| 9541 | aaggcataaa actaattttg tgatcctttg agtaatatgg atttctgagg aaattctaga |
| 9601 | ttcagagaag tgctgggcca gattttggtt aaggcaatga catggaaaaa tttttcaaag |
| 9661 | aagaggtaaa aatatgtagt ggagtttttt tgtttLtttt ttttcataat caggagtttg |
| 9721 | agagggtata atggaagttg ttaggttttg acgggaaggc gagggttaaa gaaagacagg |
| 9781 | gagagagagt tagcagctct acagcaaaca caagttttat gtctagcaca agacctgcgg |
| 9841 | aggttgggga ccagcttaat gccaaagccc actgccgctt acaggctggg gtacttatag |
| 9901 | gtgtgggtgg gaggggtctg ggcagtatgg cttgctgcct ggcaggatgt tgataagatg |
| 9961 | ttcccatgat gagatggttt ggtctttgtc ctggcaaaat gtgatagtga tgttccttgg |
| 10021 | gctttttccc agcagaatat ggtaagaaat tcaggtggtt gagcaaaatg tttctcatgg |
| 10081 | cctgaatccc catggaatgt ttcactttga ccaatgtctg cgaaatgatg gggggcttac |
| 10141 | aaagtggtac agtttggatt aacattcttg ccttctactt cagtctaaaa ggaagagggg |
| 10201 | cattgttgat tatctagctg cttcctgctg aataggggca ctgtaatcag ggcttgggtt |
| 10261 | ttgaagcagt gggtgtccaa cttcagagtt gttttcctgg agatgttgat accagacttg |
| 10321 | gtggggaaag atggtatcaa tgtgtttctg ggtggctgcc tagacaaggt agttctgcct |
| 10381 | ttgggagata aagtgggata tgaaggtaag tatacatggg ccaattgtga gtattaggag |
| 10441 | gaagattagg ggccctaaga aaggggtaac cagatatccc tgtgccaagt gtagctgaga |
| 10501 | tgcttagtcc tgctaacagg ggaatgaagt gtatagccca cttggtgtgt gtggaggagg |
| 10561 | aggaaatagg gattactaaa gaacctgaat ggtttgattg ttaggcaaaa tgttaatgtt |
| 10621 | tggagattga aacaccaggg tacatgttcc tgaccaactt gccagtaggc aaagaagaga |
| 10681 | gtttgttcca cacagaaaga aaagactggg ggttgacagg caaagttgta agtgaaggca |
| 10741 | gtgagccatg agaagccggg ggttaaatcc tgaacaaact tttgtcccaa cttttttgtt |
| 10801 | tttccaggtt gaataactgt taacaaggat agtccctgtt agggcctgat agggatattg |
| 10861 | gtggtgaagg ctgtctggtt ttagacagag agagagaaat gggtttttgt taacagccat |
| 10921 | tgcggtcctg acagggaaga agaataaagg ttgcagttga gggaattgtc tgtgcatttt |
| 10981 | tttagggagt tcccttaagt tggatgcatg gagaacagtg ctggcagaga agggaggggt |
| 11041 | tgggaaggga tgttttatgg aattcagttt gtaacatgtt tagtttacag gccatgagac |
| 11101 | gaaagagggt gccagccctg cgtgcgatgg tgcaggtagg tttgatggat tgcagggaaa |
| 11161 | ggtgtttagc caagcaagat tataaacagg ttttgggaag ggactggttg agtaggtgag |
| 11221 | atgcaggttt actttggatc aagtttctgt ggttaggtta acaggaaggg ctgtgaactg |
| 11281 | ctggatttgt gtggacaaac aaatttaaca attggaagaa agaggagagt gtgactgatt |
| 11341 | taagaggtaa tgtgtgaggt tgacaaagga agcccaactg ccagaggtta tgggatttag |
| 11401 | agtatcccac agcaaagaca aaaggggaaa aagaagattt gagtacRagg gaaattttgg |
| 11461 | aaggtRccct gcagccatag ctcctgggtt aatgtgagga attggagtgg actatcoagg |
| 11521 | gatgtgggga gggaatatca ggaaagatct gaaacaaggt gtgagataac agatttgaac |
| 11581 | ttggaggcag agagtgtaat ggaccagggc cttctggact ggcaacttct ggaattcttg |
| 11641 | ttaagcatag tgaggttggt cctatgaagg aggaagaata ttttgggggt gagaaaactt |
| 11701 | gtggatgtaa gtctggtgct gtttttagtt tggaatggtg gatttagtgt ggaaaggatg |
| 11761 | ttagttttgt tgccacagga gtagtcagga ttacctggtg aggacccgtc cacttaggtt |
| 11821 | ggagagggga gaaggaattg gtgatttaga cccagttccc tggttgtagg ggcagggagg |
| 11881 | agtgtttgga ggatggactt ttaggctggg gcaagtaagc atttgcgtac cgtcttatta |
| 11941 | gatgttgagt gaggtataat gccagccaag tatcctgtat caaRgaggtg gaggggcggg |
| 12001 | ctgggggcag atacagggag attttygagg ataaaagggc attggtacat gaattcaaat |
| 12061 | gggcttaggc tgaggggcgt gtgagcaatg gcttgtaaat tcatgagtgc cattggaaga |
| 12121 | agtgaagttc aggccatttt aacctctagg aagtttggtt atttgttgtt ttaaaaaggc |
| 12181 | attgacctat ttacttttat ctgaagattg ggggcagtaa ggaacatgaa aggcccattt |
| 12241 | aatgtttaca gcctccacca actgttggtt aacctgtgaa acaaatgttg gcccactgtc |
| 12301 | taaccggatg gaggagggga gtccaaacca ggggatcata tgggtgagga gaatagaagt |
| 12361 | gatggtgtgt gccttttcgg tggtggtagg aaaatctttt atccatccag agaatgtatc |
| 12421 | tacaattgtc agaaggtacc aggcccattt tatggggacc atgagggtaa agttgatctg |
| 12481 | cccgccctgc cctggcaggt gtccttgggc ctggtgtgtg gggaaaggag gtggtttgat |
| 12541 | agctccctga gaggaagttg tttgagtgca aatggaatat gccttagtaa tacctatgag |
| 12601 | attggcagcc atggcgggaa aatatatata agcttttaag agctagagta gggggcagta |
| 12661 | accggcatgg aaatggttgt gcacatatga aagtacagaa agttcctggg acttgggcaa |
| 12721 | gacaatttta ttattggggt agaaccatcc tcctccccaa atagcaccag cccgggtgag |
| 12781 | tgaggtttgt tcctcctggg tatatacagg gtgtatgctg ggaaagatgg gcaataatga |
| 12841 | tgaggtttta agggctgcct gctgggctgc agagtttgct gagaagtttt ccttggttat |
| 12901 | ggcatctgta gccttttgat gtcccttaca atggataaag gtggccttta gtggtagttt |
| 12961 | agctgcctcc agcagtttgt gtatgagttt gccatttact atgggggttc cttttgtagt |
| 13021 | taggaaacct ctttcctgcc agattaaggc atgagaatgt aggatgtggt atgcatattt |
| 13081 | ggaatcggtg taaatgttaa ccctcttgcc tttactaggg tgaaggccct ggttaagaca |
| 13141 | actagcgcca cctgttgaga ggtagtatgg agcgggacag tgctggattc taggagttta |
| 13201 | ttttcagcaa tgatggcata gccagctgct ggacatggct ccctaaaaca gctaaaaaga |
| 13261 | tcttccatta atgaatcatg tgggtgttcc ctgcaaaggg gcttttgaaa tgtgttggaa |
| 13321 | gggggagaag agggagttta agaggtccag gcaggagtga gagagcttag agtcagaggt |
| 13381 | gtttacaggg aagagggtgg ctaggttgag agctttacat ttctagaagg tgattagagg |
| 13441 | gttacctatg aataaggcat gtatctatga ataaggcatc ctgtaagcag gatggtggga |
| 13501 | gggatagaag agatcaatgg ctcatgagat cctgtaggtt atgggaagat gcagtagtaa |
| 13561 | tgtgttggta gagagtaagt ttctgtgcct ctgaggcaag taatgtggct gcacccaaga |
| 13621 | tttttgggca ggatggccag ccttggatga tagtccattt gttttaagag gtatgcaatg |
| 13681 | gcttgtggag cattgccata tgtttggcag agtagtccaa gagcaaggcc ttggtcagaa |
| 13741 | tgtacacaca gtgaatgggg ccagcccctc cacacctgtg ggtatttctc atcaggcagg |
| 13801 | aegagagact gagaaaagaa ataagacaca gagacaaaat atagagaaag aaaagtgggc |
| 13861 | ccaggggact ggggctcagc atacagagga cctgcaccgg cccggtctct gaattccctc |
| 13921 | agtatttatt aattactatt ttcactatct cagcaagagg aatgcggcag gagaagaagg |
| 13981 | tgatagtggg gagaaggtca gcaagaaaac atgtgagcaa aggaatctgt gtcgcaaata |
| 14041 | agttcaaggg aaggtactat gcctggatgt gcacgtaggc cagatttatg cttttctcca |
| 14101 | cccaaacatc tcagtggagc aaagagtaac agcagcattg ctgccaacat gtctcgcctc |
| 14161 | ccgccacagg gcagcttttc tcctatctca gaattgaaca aatgtacaat cgggttttat |
| 14221 | accgagacat tcagttccca ggggcaggca ggagacagtg gccttcctct atctcaactg |
| 14281 | caagaaggct tcctctttta ctaatcctcc tcagcacaga cccttcacgg gtgtcagcct |
| 14341 | ggggggcggt caggtctttc ccatcccacg aggccatatc tcaggatgtc tcagtgggga |
| 14401 | gaaaccttga acaatacctg gctttccagg gcagaggtcc ctgcggcttt ccgcagtgca |
| 14461 | ttgtgcccct ggtttatcaa gactggagaa tggcaatgac ttttaccaag caacctgcct |
| 14521 | gtaaacattt tgttaacaag gcacatcttg cacagcccta gatcccttaa accttgattc |
| 14581 | catacaacac atgtttttgt gagctcaagg ttggggcaaa gttacagata aacagcattt |
| 14641 | cagggcaaag caattgttca gggtacaggt caaaatggag tttcttatgt cttccctttc |
| 14701 | tacacagaca cagtaacagt ctgatctctc tttcttttcc ctacaataca gagtagaagg |
| 14761 | cttggtggag ttgggcagtc ccagtactgg ggccattaaa aagggcattt ttcagctttt |
| 14821 | taaagtggga gttgatgggg caagctgggt tgaggggttt taggatgagc ccatgtgagg |
| 14881 | ccatatagaa cagcttggcc agcaagccaa agttgggaat ccacagatgg aagcatccca |
| 14941 | tgcagcctga gaaggagagg aggttatttt tcgtgtgggg aagaggcatg tcccaaatta |
| 15001 | gctcctttca ttgggttgga atggcctgag aattaggggt taggacaagc ccaaggtaag |
| 15061 | tgacctgcat ttgggctaac ctgagccttt gtgggtaaga ccctaaaatt tacttggtat |
| 15121 | gtcccacccc aagatagggg tagggcactg agggataaac aggaaagagt gggtgaaggg |
| 15181 | ggtgatgaat aggttgcata atagaggacc agtctgtttg tgcctagagg ggattctatc |
| 15241 | gactcccaca atagagatag aagaactgag gaggggctca gaatattctg gtaaaattga |
| 15301 | ataactagcc tcatatttaa tgggaaagac atgggcttag cagacactga cagcattact |
| 15361 | cttggttcca aggtggtgat ggtggtggga gtggeggatt ctgggcccta tcagtcttca |
| 15421 | ggtagaggtg gtgaagtggg ataaagcgtt ttgctgagca cagtctgaga tgtgctccct |
| 15481 | gtcctccagg gtgagggagg aagagaggat gacgtaagtg tcatgccagg taatttataa |
| 15541 | gattgagtga catacagaaa ttccttgtgg aaggaggtgg gatccatgga aaagaagcca |
| 15601 | agtctttttt caagctggaa gaggtcagct agggagaagg aaacataaac tcagattata |
| 15661 | ccttcggccc gcgtgacttc ccgcaagggg atgactttgg aaggcctttg ggtgtatgcc |
| 15721 | tggttttcag aggaaggggg ttaagtgtga gactgggtat gcggtggaga agaaaggggg |
| 15781 | aacagagggc aacaataagg gaKtgaagga gccggagctg aaggaggagg aggggatggt |
| 15841 | ggtgggagaa ggactgaagg aggggttttg ttgtccgggt tgaaattaga ggaggaaggc |
| 15901 | ttgtctgaag gaaggggcat ttttgggttt ggaggttcca gattagacat ctgagagtgg |
| 15961 | tgtctggagg aagttttgag gagccttgcc ccatactggt ggctggaagg gtgagaaata |
| 16021 | tagaggaggg gtgtgagtac cctggagggg agtaagtacc atgattcctc ttgggaggag |
| 16081 | ggacataaga aagccagggc atccctgcag ctcttcacag tcctctggag atcagagtga |
| 16141 | ctggagtggc gagcattctg agggcgtccc ctgagaaggc agaccatggt cacctggtga |
| 16201 | ccagggagac ctctcaccca gcactgggat tttctggtga aggagcagaa gcatgaKagg |
| 16261 | aatctggaga ggaggtaaag ggagactcac ccattaattg gagacggcgt tggacgtgat |
| 16321 | gttctgcaat gggggtgatc cttgccggag ccacccagaa agggggagaa agaaaaaaga |
| 16381 | ggctagagag tttgatcagg atctagaggt cagcccaggg ccaaaggaga gagaataaga |
| 16441 | gaaaaagggc tgcaaagagg gagtctactc aggatctgga agcaggcctc agtctaattt |
| 16501 | ttgctgcttg ctgctttcca ggttgcaaga gaagacttac ccgtttagaa cccaatctat |
| 16561 | gtcctgggtt tctgcgccaa aatgtagtgg agtttttttc ataatcagga gtttgagagg |
| 16621 | gtacaatgga agttgttagg ttttgaaggg aaggtgaggg ttaaagaaag agagttggat |
| 16681 | gctttgcagt aaatgcaggt tttatgtcta gcccaagacc tgcggaggtg ggggaccagg |
| 16741 | ttaatgcctg aacccactgc cacttacagg ctggggtact tacaggtatg ggagggaggg |
| 16801 | gtctgggcag tatggcttgc tgcccaacag gctattgata agatgttccc atgatgaggt |
| 16861 | gacttggtcc ttgttccagc agaatgtgat agggatgttc cttggacttt ttcccagtgg |
| 16921 | aatgtgatca gaaagtcagg tggtcaggcc gatgtttctc acacagagaa ccacttccac |
| 16981 | aagattaatt tccatactcc ttccaccact tgtttgaacc ttcagcttta tcctattaaa |
| 17041 | ttcaaaacaa tcttttaact ctaggcaaca atttatattt ccatggcttc ttataacctt |
| 17101 | ttagtaaaac acacacacac agacattttt ccaaacacac cttgcatgta aaaatatttt |
| 17161 | tagtagtatc aattacatgt tataatgatg actcttagca atcttacttt taatgtaaaa |
| 17221 | gctggtaagt tatgttaatt atgtactagg tgctgataag gtcttactat tccagcatag |
| 17281 | ctaggggtgt ggccaactcc acatgtcccc aggccttacc tagctgtaaa gcaggcaagt |
| 17341 | caaacaattt tcaaaagcca aagaagcagt ttatgacctt aaggcactta gcaaacctaa |
| 17401 | tatttgaaca taatttagat gacatgtttg tattttgaag acatttgtga aaactttaca |
| 17461 | gaggagataa acagtgactt tcacgtctca tttagccagt ttgcacagag agaaagaggt |
| 17521 | cagagtctga ctggtaggaa attcttatcc ttttgccagt aggccaggtt tctgggtttt |
| 17581 | ctctccctga gcagccctag tgaMcctgct caactgtatg caaacaaaca cactaccatg |
| 17641 | aattaagaac atttacaaat agtttacaaa ttttggagaa attaggcaga gagagaaata |
| 17701 | tgacttaaat tctgtttaca aaagtatact caacatactt aaaatatcaa taagcctaaa |
| 17761 | ctccaaaaag ttagtttaag gttaaaaagc tgatgtgttc cattaactcc tgtgggtctg |
| 17821 | acaaaggtag cctaggaatt ccagataaat ggaacaaatg atgacttgct agaaatgcac |
| 17881 | aggaaacaaa ataactatcc acaaaaccaa ataaaagcct tctagaagca aaaataaata |
| 17941 | aacaggaaag caaccctaat tttttcttac tcaatttacc ctggaggcta aagtgttgcc |
| 18001 | cagggcccca aaagagccac ataataaata ttttattcct gatacacaat tcaatatcct |
| 18061 | taagttcacc aatatcatca tacatcctgt gcaatcaaga agttcacttt aggcccatga |
| 18121 | ccaataagca ctccagtgcc agcactatcc atgcaaaaca gtaaacatag tgtgaagcag |
| 18181 | tgcaagcata tctgggaaat ttggctccac actaaatcca gcttcaagct taactatata |
| 18241 | aaaaaaagaa ttgccaaact gccaatgcat ttctttacaa tagttattat tttactttaa |
| 18301 | tcaagactaa gagctttaac tacgaaaatg ttaactagcc caatgtctcc aattctttat |
| 18361 | catgttttaa agaatatttt attatctaaa ctttttccac atccttctcc cctaatcact |
| 18421 | ggttctttac tacattgttt cacaaataat tttttcaaat gtgtaatttg aactaactbt |
| 18481 | tagataactt cttaattaga caaaattatt ctttttctca gtaataacac aaactttttg |
| 18541 | gcacattttg tatacagaac tctgtgttaa ctagaagtat tatccttagt aacctaaaat |
| 18601 | tttagtgaaa ccctaaaaag aaatactgaa ctatcagata tgggcattta tagataagaa |
| 18661 | caattccaca atttttagaa acatatttcc ccatatcaca acccttcctt aattggaaat |
| 18721 | gacccaaata ttcaatgagc atcaaaatta attttaagct tttaatttac acaaaaagtt |
| 18781 | tacctaaaac atttatccca tttactgtac tcaattcttt catttttaac agtttatcta |
| 18841 | gattacttct gtaaactgag atattagaca ctatcactta aagttagtta tttccttgct |
| 18901 | aaccatgttt ctaatagcca gtgaatatca agtgctcacc taaacctgag agtctcagaa |
| 18961 | ttaaatatat aagtattttg ccaataactc agaagacgta gctaacatta aattgctctt |
| 19021 | atttgtcaaa aaaaaaaggc acattttgtt ttggctgggt ttgtagtttt ataacattct |
| 19081 | atgccaaaca ttgacatctc aaaatattta gcaaacacaa acataaaatc cagacaaaat |
| 19141 | gtatgctgac aattctgaag gcatttctat ttttattcta ccaataattt taaagccagc |
| 19201 | ttgtttagta aagttatact taagtcactt taacttaaaa atcgcttagg cttattaatt |
| 19261 | tatgagcgct cttttactta taagccaatt tggcaaacat aacatataag tgtacataca |
| 19321 | aataaacaca tctagacatg tatacacaca cacaaatgaa gatctaatag cttgaaaccc |
| 19381 | tagccatgag atagcaacac aagcttaccg gttttacttt gtttgcccca gtaggtaatg |
| 19441 | caatgaaggc tgtgaaccaa aattttgggt aaagcaattt ccatggcagt ttgatttttt |
| 19501 | ttgtttcttt ttcttttttt tttttttctt tgagatggag tcttgctctg tagcccaggc |
| 19561 | tagagtgcag tggtgcaatc tcggctcact gaaacctctg cttcccaggt cctggttcaa |
| 19621 | gcaattctcc tgcctcagcc tcctaagtag ctgggattac aggcacgtgc caccatgtcc |
| 19681 | agctaatttt tgtattttta gtagagatgg ggtttcacct tgttggcctt gttcatgtct |
| 19741 | tgaactcctg acctcgtgat cYgcccgcct cggcctccca aagtgcaggg attacaggcg |
| 19801 | tgagccaccg tgcccagatg gcagtttgat tttttttgtt tttgtttttg agacagagtc |
| 19861 | tcactctgtt gcccaggctg gagtgcagtg gcgtgatctc agctcccact gcagtctccg |
| 19921 | cctcccgggt ctaagtgatt ctcctgactc agcctcctga gtagctggga ctataggcac |
| 19981 | gcaccaccac gcctggctaa ttttttgtat ttttagtaga gatggagttt agtagagatg |
| 20041 | gggtttcacc acgttggcca ggatggtctt gatttcctga cctagtgatc cacctgcctc |
| 20101 | ggcctcccaa agtgctggga ttacaggcat gagccactgc acctggccct agcagtttga |
| 20161 | tttttaaagg tgaaacctcc ccagactcca aagaacacta agaccaaaca gtaccaaagg |
| 20221 | agaacatcac tctttcacca ggccctctgc ttagaatagc agcaccaaag cctggatata |
| 20281 | tgcaatggca atgccatccc agttttccat tcaacagtaa actccgcatt ccaaacaata |
| 20341 | ttggggccaa atagcattgc aactgcgaga taaaattcta aggagagttt aatactagac |
| 20401 | ctcagaacct ctgccaaggg catcctcttt ggagaggttg aggtccggag gattcccctc |
| 20461 | tggggcttcc cctagtgggg cccaatctta agagtgtcag acgtctctga ccttaggtgg |
| 20521 | gcaccagtcc tgcttgcatg ttttccctct aaagggaatg gcctactatg agctttcatt |
| 20581 | ttgtccctgg atgaaggcct tgacttttag catccttata atttgataag gccacacttt |
| 20641 | cccatgtttc ccattccatg aacttaataa ctggaggctg gatgtgtttc ttttgctctt |
| 20701 | agccagttga ctagggaaac ggaagaatta cgcataagaa aaggtttaag ttgcctgaaa |
| 20761 | caggcgagtt tgctctgagc tgtgccacac atagggatca gggaccacac ccagaaaaga |
| 20821 | tttaaaaaag agtccttctc ccttctgggc agggcaatta tccccattca ctccatggtc |
| 20881 | ttcaggcaat attggggatt gatcccagcc aatttccctc aatttccaaa gagctactag |
| 20941 | gaaacagctg ctaaaagacc aataaagaag aaaaggaaaa aaaaaaagaa aaagacccag |
| 21001 | gtcccttagg caaaccaggt ggtggcggtt aggcttcttc acatgaaaac cccttagtat |
| 21061 | tcaccagcca tggccagaaa cctgcagttt acctccatgt ttacacgctg ctcaccaagg |
| 21121 | gtcccgagtg ggaaaggaaa agagagagag agatttccct gtatgtaaaa agggaaagga |
| 21181 | gaaaaataaa tcccaaactt tgggcttacc ttctcctggc tggctcacca aaatatgtta |
| 21241 | ccagtggagg gtgtccaggt gcttggcatt ttgaacaaag aattggacaa aatgcacaaa |
| 21301 | caaagcaagg aaagaatgaa gtaacaaaag cagagattta ttgagtacac tccacagtgg |
| 21361 | gagcaggccc aagcagccac tcaagggccg ggatacagga tcttcttggg tccaaatacc |
| 21421 | cgctggaggt ttcccattgg ccacttggtg ttcacgccat gtaaataaag tggtggcccc |
| 21481 | caatcagtct gattgtttgc agacggaaac caatcagagg ctgaagtgaa gttacaaagt |
| 21541 | tacactctta ggcaaacgcc tggttgccaa aagcaaccaa tcagaggtat tttctttttc |
| 21601 | tttttctttt tttttctttt tgagacggag tctttctctg tcacccaggc tggcgtgcag |
| 21661 | tggcccgatc tcggctcact gcaagctcca cgtcccgggt tcacgccatt ctcctgcctc |
| 21721 | agcctcccga gtagctggga gtacaggtgc ccgccaccac gcccggctat ttttttgtat |
| 21781 | ttttagtaga gacagggttt caccatgttg gccaggatgg tctcgatctc ttgacctcgt |
| 21841 | gatccaccct cctcagcctc ccaaagtgct gggattacag gcgtgagcca ctgcgcctgg |
| 21901 | cccagaggta ctttcaatta accatctgcc atgcagaaaa ggtgggagtt tgcaaagaga |
| 21961 | gtagcctctg gtccttttgt tacttaggca tggaaagtta gggttttcct ttcaatgtag |
| 22021 | ttctacgaag tcagcgtgaa atggccttag gtttcctatt ctcctgcctc actggtatcc |
| 22081 | aagggactct gccattaggc tagctgtgga gggatggaga gagagctgag gcctcaaatg |
| 22141 | ccagtattcc ttccttccag atttgcgtgt aagaccagat aatcttccag accaaacagt |
| 22201 | atctgttgat gagtattagg ctcgaatgag aaggagagag aagtggagca ctggattgaa |
| 22261 | gagaaagcaa aaaaccttcc tagatcaaca aggaaatagc ctcttctaga atccagatag |
| 22321 | ggccctggac tcctgtggga taatgtagtg ggtgaaaaaa ttggtaRtga agtcctcgga |
| 22381 | tatggggatg cacttggaag tcttgggaac aaggaagaga gacatcctgt ggtggtgatt |
| 22441 | tttagatcca acacccaggg gcttgggtgc ctttgggggg attagtagaa gagatcgaca |
| 22501 | cattgaggga ggttcaggat aactgtctga aaatgagaga tttttattgc ttatactgat |
| 22561 | gtcccaatag ggtctgtatg tgcttcaaat aggtagaaaa gaatgaagtc cgagcctgaa |
| 22621 | gataacaata tcagtcagaa ttttaggatt gtgaatacct gtccacagaa atagctgggc |
| 22681 | cctgagcctc tgctgatgat gtaggaatRg aggcagttaa taagctttaa agtcataggg |
| 22741 | tctcagggct agggtatggt gaggacactt tgccacccct aaggttcaaa gcctcttgga |
| 22801 | ctggaggagg ttgttaacag atattctagg cagaaaagct gtgtcaggtt gtggtgtatg |
| 22861 | aagagggaga atttgacacc aaatggtcag ctggccatct gctcacctgt tcccctgtgc |
| 22921 | tccctactcc cacagggtct gcttacagaa caaggcacag caaggaggca agacacaagc |
| 22981 | aagaaatgtg cttgatgtta cccactgcag tccttcttcc actctggagg attctgaaga |
| 23041 | ctcagaagag ggacaggcaa tggattcagt ctaaacccca ggaccaggac actgcctcag |
| 23101 | cttcgaagcc tggatgcaat gctccagggt ataaccactg cccagaagca cagctagtac |
| 23161 | aagctgcagc ctcaatccag gcaaggagcc aactccaaca ggtcaggctc caagggatga |
| 23221 | agactgaaaa catgaatgtg ttgtgccact aactctagcg tttgcttttc cttgtgtttt |
| 23281 | tgcatgcatt gtagaccttg gcatcagcat ttggctagct ttaagacaga aactgagaag |
| 23341 | ccttttgtgt tttatgcgtc aacttgactg ggtcacagca tgcccaaata tttggttaaa |
| 23401 | cacgatttct aggtgcatct gtgaggattt tctggacgag actaatattt gaatttgaat |
| 23461 | cagtggacta agtaaggcag actgccctcc ccagtgtggg tggccctcat ccttctctct |
| 23521 | gcctgacact cttctagctg gaacatttgt cttctcctac cctcggactc agactatgac |
| 23581 | tggaattgat atcattgact ctcctagttc ttcagactca gattgctctc Scgggacttc |
| 23641 | tcagcttcca aatcacatga accaattcat tataggaaat tttttWatat atcctattaa |
| 23701 | ttctgtttct ctggagaacc ctgattaata cagatttatt ttcctacact ccagtaactc |
| 23761 | tccactactc cctccctcaa cccctggtaa ctatcctact ttctgtctct ataaatttga |
| 23821 | ctattctcag tacctcatat aagaggaatc atgcagtatt tgtccttttg tgactggctt |
| 23881 | atttcattag cataatgttt tcaaggttta ttcatgttgt agcatgtatc caaatttaat |
| 23941 | tcctttttga ggctgaatga tactccattg tatgtatata ccatattttg ttcattcatc |
| 24001 | cattgataaa catgttgttc ccacatcttg gctattagga ataatgttgc tgtgaacatg |
| 24061 | agtgtacaaa tatctgagtc cttgcctttt tttttttttt ttggacacag agtctctctc |
| 24121 | tgttgcccag actggagtaa agtggtgtga ttctcggctc acggcaacct ctgcctcctg |
| 24181 | ggttcaagtg attctcgtgc ctcagcctcc ccagtagctg ggattacagg cgtgtgccac |
| 24241 | catgcctggc taattttttg tatttttaat agagatggga ttcgtcatgt tgcctaggct |
| 24301 | ggtctcaaac tcctgagctc aggcaatttg cccacctcgg cctcctaagg tgctaggatt |
| 24361 | acaggcgtga gccaccatgc ctggatcatt gcttttcatt attttgggta tatacctaga |
| 24421 | agtgggattg ctggatcata tggtcattct gattttttga ggaaccatca tactgttctc |
| 24481 | cacagtgtct gatgacctac agttttgagc ttagcctttc taaaatagtg gggaaaaaaa |
| 24541 | tgaaaagaaa acaactagaa gtggaagtaa tttgtccttc attaagccag atatacacat |
| 24601 | cctgattcaa gcttgaaaac aatatcacct ccttcgctga tatgtaagta cctttttata |
| 24661 | atttaaattt taagttatga ttaattagaa acttaaaatg tatagtctgt atatgcactg |
| 24721 | gatgtccaca aagtgaccct tggttgctaa atctttgaga aaggctgagt agacactgaa |
| 24781 | cgaatgtata tccaacttac ttgtcaactc ctccaagaac attccttacc ccctgaccca |
| 24841 | agacatgctt tctatgagct ccaacagtgc ccctagtctt caacagctac cacatctgtg |
| 24901 | ctcgtcacac tcttgtcctt ctcctagtct caactgtgag ccatggatgg cagaggccgg |
| 24961 | tttttaccct tcaggtctcc agttaggcct gtgatggttc tagggttttg tttaggctgc |
| 25021 | gagtctgatt ggggctagga caaagggtca aattcaggat gaaaatcagc ctggccaaga |
| 25081 | gcagtggata catagccccc atcatcccac ttctcagatg ctcctagaac tccagcccgg |
| 25141 | gatcctttat cctctgtata ctccaccttc tccccataca agaccctctg cagagagcag |
| 25201 | gcacaaggag cagataaatg atggtcgatg ctgcctgtca attcagatgg gcctggcata |
| 25261 | acggttaact aatgggtttc ttgtttgatt gataggttct ctcaaaacta aaggagttaa |
| 25321 | atttctttaa tagcaataca tgctcattgt taaaaactca tatcaactga atttcccttg |
| 25381 | aacactatta acatttagtg tattatcctt tcacatttgt atagatattt agaaaaatgg |
| 25441 | agtgtagtat atacgtttat atatttttgc ttagcagtac atcatagaca tctatttcat |
| 25501 | tatattcaaa tggctcaata atattcccct gtgtagctgt accataatct agccaaactc |
| 25561 | ctatgtgggg gcctggtttg tatttgattt ttcagtaaYa taaacaatgc tgagataaac |
| 25621 | ctctatcagc tagattattt ctattgactg attttaatac atgattggaa ttttaaccac |
| 25681 | aagggggaag caggcaacca tgggaagaca ggctaagatt taggataaag aaaaagaggc |
| 25741 | tgggtgcagt ggctcatgcc tgtaatccca gcgctttggg agggtgaggc aggaggacag |
| 25801 | ctcaagccta ggagtttgag gttacacaaa gctataatca cactactgca ctccagcctg |
| 25861 | ggtgacagag caaaaccctc tctctcaaaa aaaaaaaaaa aaaaaaaaaa cgatttagaa |
| 25921 | agttatctca catacataag cttaccccaa ccttgtgagc aggtagtagc attagtatta |
| 25981 | gcctcatttt ttggaggcaa agctgatgat ttcattatca ggcgctcttc tgtatttcca |
| 26041 | aataaattga cagcagtaca acattctgcc tacattcagt tcaagtccag gtgctgtgct |
| 26101 | ggggacacta tgaggcatgc agaaataaag aagacaatcc ctgcccctca gggggtcatt |
| 26161 | atcaggtgtg aggacagaca cacctaaata accaagtgcc cactgtaaca gacaggagag |
| 26221 | cccaggaagg taggaaattc acttcgatag tgaggctggg cacagtttta tggaaggggt |
| 26281 | atgctgacaa taagaggtaa ggtgtattta gcctttactc tgtgcatggc actgtgctaa |
| 26341 | gtgctttacc tgcataatct cattaaaact tccccacagg ccaggcaccg tggtttatgc |
| 26401 | ctgtaatccc agcactttgg gaggccaaaa caagaggatt gcttaagccc aggagtttgc |
| 26461 | aaccagcctg ggcaacatag cagggccctg tctcttaaaa acaaaccaac caaccaacaa |
| 26521 | acaaaccaac aaacaaatcc ccatagacct ggaaggtatc ctaccctttt ttacagagaa |
| 26581 | gaaaactgat ggtcagagag gttaagaaac cagctaccgt ggcacaacac tggaacgcgc |
| 26641 | gtccttaatg tgaattcagg aggccctaca agggttggta gactttcaac aggcagggct |
| 26701 | gggagagaga cttctaagag aggaaacaca gaaacgaagc cttagaggca agaaatacag |
| 26761 | gcacgtctgg gaatggcaag gggatcagcg tgcctggggt gtgaaaaagg aggaatctga |
| 26821 | agagatcaaa actagagaaa gaaaactgag tcagagtggt gaagactgtg agtagcaagg |
| 26881 | caaggagatc ccgctgtatt ttgtattcag gtgaaaccaa ctgaagataa tgacatcatg |
| 26941 | tgtgctttat gaggattaat ttggtgcatt gcMtaagaga acaaggagac tagttagggg |
| 27001 | cttgttaaaa tagtcctggc caaagataac tagcgaagga atgagaacca gaagacatta |
| 27061 | cagagataaa atccacaaga tcttattaga tgctggggaa agaggaaaga attgtgatta |
| 27121 | agattttcat gtttgggcaa ccaggagaat gtgagtgtgt acaccaggag aggggctgga |
| 27181 | cagaagggag aggcgagttc acttttcttt tatgagctgg aaggaccaca gggacctttg |
| 27241 | agtggagatg tttagaggag agttagaaac aggaagctgg tgatttagac atagtacctg |
| 27301 | agaatcagaa gaaacccagg gccgggtgtg gtgtctcacg cctgtaatcc tagcactttg |
| 27361 | ggaggccaag atgggagggc tgcttgaggc caggagttca agaccagcct ggtcaacata |
| 27421 | gtaagaccct ctctatatag aataaaataa aatttaaaaa aagaagaagg ctgggtgcag |
| 27481 | tggctcacac ctgtaatccc agcactttag gaggccgagg cggtggatca cctgaggtca |
| 27541 | ggagttcaag accagcctga tcaacgtggt gaaaccccgt ctctactaaa aatacaaaaa |
| 27601 | ttagctgggt gtagtggcag gtgtctgtaa tcccacctac tcaggaggct gaggcaggag |
| 27661 | aatcacttga acccaagaga cagtgagcca agattgcgcc attgcattcc aacctgggtg |
| 27721 | acagaacgag actctgtctc aaaaaaataa aaaataaata aaaagaagaa gaaatgacca |
| 27781 | agggagctgg ccagggaaaa acctacactt aagattcagc aaggcatgtt agaccctgat |
| 27841 | aacctgcaat tccacagcca ggtttctatc ccagagaaac aaaatcacac gtgcacaaga |
| 27901 | gagcgtatga aagttcacag cagcattgtc tgtcttggtg aaagcctggg tcagggagga |
| 27961 | gcaggcaaat aaaccattaa acatccacat tgtgcaatag ttaccaatta agtagatcaa |
| 28021 | aggtattgac atggacagat cttcagaaca tacagtggat gagaaaaaca acatgtagtg |
| 28081 | caataataat gtataaaaca ttacttatga aaaaagaaga aaagccaaac aatcctgtca |
| 28141 | catatatatt ttaaatgatg tatgtgtagg catagagaaa attctggaaa gctaaatcca |
| 28201 | tgctaattgt gataaagggg aggagttggg acaaacatga agagcagaag gggttaaatg |
| 28261 | gggttttagc atagttgtca ggttttaatt ttataagaaa aatgtaatca tctgttactt |
| 28321 | atgtgaataa aaattctcaa aatattttta aaaagaaaga ttcaatggga agaggaagca |
| 28381 | atggaataga caggaataca ttcctagact ctaggtagga cactgttact gggaacactg |
| 28441 | ggagaagttt ctggaaKgca aggtggaaaa aggatccact gcactcagca acgctgcaga |
| 28501 | gacagcagcc agcaggatga gtcctgtgag ctcagctggt tgtcaacacc tttcacttac |
| 28561 | agccactagc tcctgtgtcc ttgaaaagtc gccatgtccc accactgccc ttgaatgaga |
| 28621 | tggagacttt ctgttctttt ttgagttgta aacatccaaa agcatttcRt caatttcttg |
| 28681 | tatttagaca ttcccttcct ttcccagaaa ttccacagtg atattctcag tgttctcttt |
| 28741 | ccagtccttc aagctgtgta tgaaatctac ttcttcccag tccagctcca gcaaatacct |
| 28801 | gcctcctctc ttgttctcca ttcaaaaaac ttccaatttg aggtcatttt taagagcatt |
| 28861 | actgtggtgg tggcggtggt ggagtttata gaatttctca tgggacccac agcatggatg |
| 28921 | tggctaacac tgactgagca cacaaaggtg caggcactgt gctaacttca taggaagtat |
| 28981 | ctcggttaat cctttcagca accccagtag gtcagtacta ttataacctc caaggtcttt |
| 29041 | ctttcacagc tgggacttag tgtggctgag cgacctaagg aggtaagtgg cagtaagctt |
| 29101 | gtggtgggtg actctgtgtt tgtctcacta tgtgagtttg tcctaagtga tcaatagggg |
| 29161 | tggggactgg actagaaaca aggtctcagg tatgcagggg tgttcagttt accaaatgcc |
| 29221 | tttatgacca ttaattcatt tgatgtcacc acagtgcttt aatgacatgg gtcattaggg |
| 29281 | aaaataatga gatgagtcaa gctttgtaat tcccttaaag tggtcaagtc tgggttaagg |
| 29341 | gcttttaggg cacattaact cactatgtcg tcttgagcaa ctctgcaagg ggtgggagca |
| 29401 | gagggggccc cctttggccc agctctctac aaatcatcta gttcctagca gccactcctc |
| 29461 | ctggtctgca aattcttggc gaggcaccct cctgctctcc aaacccaccc attccaggcg |
| 29521 | atcccttaag tggcagagga gccaggttcc tggggttaat aagcaggctg tttatagcca |
| 29581 | gcagcagtca cgggacagcc agctggaagg tcatgcccct tctgaatgtt gaggtttctc |
| 29641 | ataaacattt ccccagtgag ccttctggga gatgaggatg actctgggcc cattgccctt |
| 29701 | gctggggcag agcgccaagg ccccatactc actttcttct cctttctccc gaatctggct |
| 29761 | ctcttcctct ttcctccttc ctctcctact ctgctgtcat tccttccctt tcttcagtgg |
| 29821 | aaagaggaca cgtttcacag tagacacaca ggataactca accttctgag cctcagtttc |
| 29881 | ctcctgcgta aatggggtaa taatgggRcc ttcatctcag gatgtaaggg aaagtgctta |
| 29941 | gcactgtgtt aggcacgcac agtggacatt taaaacatat tggtcatgtc tttccccctc |
| 30001 | caccctatcc ttttgcccta atcctggcca ctgccctgtt ttctttctat tttcctgctc |
| 30061 | cagccctgct cccatttcca ccctgggcac ttccacccca ctgaYtgtgc tgctgctgcc |
| 30121 | tgggagggta gacgaagaag ccaggcttca ttcagaggag ggctgaccac cgcagggccc |
| 30181 | gcctggcctt ggcacttccc catgagaccg caccctaggc ctctctgctt ttagtaaata |
| 30241 | gcccatggga acagggggtg gagggaggat gtagaatggg agaggccaag acattttaac |
| 30301 | ctttggcttc tatttgccct taatctggct cttccaatgg cttaatgatc agagatcatt |
| 30361 | gtgtaaattg ctgtgtgact ttgggcaagt ccccttcact ctctgagctt cagtgaagtg |
| 30421 | tgggaaggaa gtgaagagca gaagttgggt agaagtgcag gtcctggaac cagaccatct |
| 30481 | ccaccacttg ctagcttgga gtacataact cctctgtgcc tcagettcct caactataaa |
| 30541 | atgcaattta tagtatacct aatttataaa agcactttta aaagtgctta gaatagcgcc |
| 30601 | tagcccatag ttggtactaa attaatctca gctattatat gcaaatggag aggtagaaca |
| 30661 | ggggtatgtg tgcatgagag gagagctgga ccttctctga gggtattcta Ragtcctgaa |
| 30721 | aacccacatt cctccttttt ggataagaag aagacagaac cttgaacaca aagtacttct |
| 30781 | cccaaggaga ccataggaaa cttgtcaaag acttttaaag ccagatggac cccttagagg |
| 30841 | tcatctggtc Yaacttgtca tgctacagta agaacagggt ggaccagaga gattcggact |
| 30901 | tatctgggac cacagagctt ttgacgagtt atagaattgc aggtggtgca ttgcagggaa |
| 30961 | gggaggcact ttaagaggac agtgggatgg gctgccagag ggctggtggg atgctagagg |
| 31021 | tcagattccc aggctccatg acctccagga ttcttagttc aggcacttac cccataacta |
| 31081 | aagcactgat gtcaccaccg gcatctttaa taattataac catgtaatga ggagggacta |
| 31141 | tatcccaagc actgcaccag acgctttcac atgcattact tactccatac aaaatcccct |
| 31201 | tgRcaaatga ggaaacacgc cttgcccaag ctgcaacaat tacaaagggg cagagctggg |
| 31261 | ctttgaatcc cggtctctct gatgccaaaa ccagggttct catgcaacac tcttttcatg |
| 31321 | tttcccagag cctagtttga ggcttgagcc tcaataaata aggatcttgt atttgtctca |
| 31381 | ctgggctggc caagcagatc ttgtatttgt ctgactgggc tggccaagcg ataacactaa |
| 31441 | tgaatMagcc tgggtcccag tcacctttcc agccctttcc accctgaccc acaagccatg |
| 31501 | atgagagacc agaccctgtg taggaaatct ttcttccagg ggccctgcct ctgtgttatc |
| 31561 | tgeccccact gtcaatggga gaagcagtgg ctgctttcgg aacaggcagg ggaggagggc |
| 31621 | ttagagcctg gggtgacRct tctggctgct gggcctgacc tccccagact ttagccaggg |
| 31681 | ccctgacttg aatgggagga gcatgaacaa acagaaagtt gtaggagaag caagaaacca |
| 31741 | ggtaaggtga aaaaaacttt tcccctccaa aaatacgaag tttatagttg atgtgaattt |
| 31801 | gtcaacttaa tatagtgtaa gaagcataag gtttaatacg gtcttcctcg ccatgatggc |
| 31861 | aggaagcaca tctgttgtat tcactaccac atgcttagtg ccccacccaa ggcctgtcta |
| 31921 | gagtcagtgc tcaataaata aacatggaaa atgaatgaaa aaaaaaaatg gagggacata |
| 31981 | aggaagagca ctgaaacatc agtcagaaaa cctgcattac agtttttagc ttcatggctc |
| 32041 | acagcaaccc ttggacaagt cacttcggct ctgtaaacct cagtttctac aagtgtaaaa |
| 32101 | tagatatcat ggcagttcct actgcacagg gctttgaaat cacagacttc cgtaggagtc |
| 32161 | atggttccat aaccaactaa tgtcaagttg aaaattgtat ttcttcagag tgaggccggc |
| 32221 | tttagcaatc tatgcaaggg gtcttgctga accagggcca gaggaaggcc agaggtgtgg |
| 32281 | ttctaagaat gaggattaaa catgagagag actataaatc tgcttggaga accgcagcat |
| 32341 | taggagggta cagcggacct ggcaggcatg acagatactc tctgccctgg agagtctcca |
| 32401 | gccctgccca tgcaaggtgt cagttagtat cagcgggagc acagatgggc ttaaatcaga |
| 32461 | agatgttaca gattatctag tccacactgg acaatagccc agtgaggggt taaaggctga |
| 32521 | gagacaaaga cccagatatt ctaattccca gctcaatgct actcttccca caccaccatc |
| 32581 | aaaggagggg gacggtggtg agagggggta tttgaagaga aacagattgg acaaaggaat |
| 32641 | gaatgcctcc atgtagcaac agcttcaaag gggcttcaga ataggagata tcttgttaaa |
| 32701 | gaaggaggaa cgagaggggg aaatggttaa aaataacgtc ttacatccat gtggattatg |
| 32761 | tttccaacta tatcatgtgt agttattgca tgtaaggtac tatggtggag agttactgat |
| 32821 | ggggcatcag aaagcccatt tcattaccca gctctgccat gtacaagctc tgcgacctcg |
| 32881 | ggcaaatcct ctctctgatc ctcaaatttt catgtgaagc atgaagcaat agtgcctacc |
| 32941 | ccatagacct atcctgggga ccaggttagg aaagggaagt ggagggaggt tgtatcctct |
| 33001 | ctaaaatgct ctgcaaatgg gcaacatggc aaagcggtca caagcaagcg tgtgaggtag |
| 33061 | accRtcactt agcctttctt ttcttttttt tttttaagac agagtttcgc tcttgttgcc |
| 33121 | caggctagag tgcaatggca cgatctcggc tcactgcaac ctccacctcc taggttcaag |
| 33181 | tgattttcct gcctcagcct cctgagtagc tgggattaca ggcgcacgcc accacacccg |
| 33241 | actaattttt gtatttttag tagagatggg gtttcaccat gttggccagg ctggtctcga |
| 33301 | actcctgaca tcaggtgatc cgcctgcctt ggccttccaa agtgctggga ttacaggcgt |
| 33361 | gagccaccac gcccagcccc acttagcctt tctacgactt tgtatcctca tctgtaaaat |
| 33421 | ggatgtggtg ggaatcaaat ataatgcatt tgcagccttt ggcacagtcc ctgacacata |
| 33481 | caaagcctgc aacatcctgt tagttactac cactagttgg gtttaaagaa ttttctcatg |
| 33541 | taatcctcac aataatccta tgaggtagga ttattattat tctcatattg cagatgggga |
| 33601 | aaccaagacc taaaaagaat tgcttaccca aatcaatagc taattagtag acagcaaagc |
| 33661 | caactagaac ccagatgccc tatcagatca ggcaggcaaa gatcagaatt agtgagtagg |
| 33721 | gcaggcagca gcgggaatgt ctgtggagtc cagacaataa tgcagccaca aagccagagt |
| 33781 | tctggtagct gcagaggcat ttccaacttt aaactctggt cagtctggag ctcagtcaga |
| 33841 | acaaaccttg tttcatcttt gatttaagga gctgagaaaa ataagctctg gaaagagaag |
| 33901 | aggggaccct gtgttccagc atcatctgcg ttatttattc actaggctga ttgcttcKcc |
| 33961 | tctctgggcc tcagtttccc cagctgtata tgaaaggttg aactagatta atgcttctca |
| 34021 | aacttgaatg aggttctgag tcacctgatg atcttgtttg aatgtagatt ctgattctca |
| 34081 | gtaggtctgg gaaaaagcct gagattctga atgtctgacg agctcccagg ccatgccagt |
| 34141 | gctgcagagc taaggatcac tctctgagga gcaaggaact agatcagctt gaaggacatt |
| 34201 | tctggtatga taatgcctat gagagcaata caccaaagac tgagaagtcg agcaaaaaca |
| 34261 | aattctgccc aaagtgcctt acagtatttg ggggtccagt gtgagggcca ggctatttcc |
| 34321 | catggaccac agaccccttt aaaaaggggc ttgatatcca gcactcagtg aggcattcag |
| 34381 | ctgcagggcc agctctccag actcttcttc ccagcccatc tcccaccctc acctgcactt |
| 34441 | cacctcctaa agggaagaga caaaattttc taagccattt ccctccccta tcagggctgg |
| 34501 | ggagggagcg gcttgttcct aatgaagaca aaggacaagg cttaagggac acttgagaat |
| 34561 | ctttttctgt ccttcctttt cctcattcat tcattcattc ttcattattc aactatttat |
| 34621 | tgagtactct ctacgtgcct ggtattctcc taggggctga ggttacagca gagaaaataa |
| 34681 | gacagatgtg gcctctgacc tcatgttgct tacattctaa ttgggaggaa aatgggacag |
| 34741 | gccataaata ggtagatttt aaaaatgaat gagatcatct aaaatcacaa aaagcaataa |
| 34801 | aagaataatg aaacaaagtg atggggtagg gggcttcttc tgtctattgt cctcaagaca |
| 34861 | aagtccaagg ttggaaaagc tcatctccac ctgcccttgt ttgcttctcc agcctcatct |
| 34921 | cctgtcactt tttgccttga actttccatt ccagccacac aggacatgga gtatcaaggc |
| 34981 | tggaagacgc caaccagcct ttgatccagt ggttctctca gccccaatca ctctttctct |
| 35041 | ttgtccctac ctcttcctct ttttccctct ttctcaccca actaacccct cctgctcttt |
| 35101 | cccagcttac tcgccactcc cactggaagc tggccctgct gccactgatc tgggctagga |
| 35161 | atccgtctgc gccaccttcc cWgatgacag catccatcac ctgtgcagcc gtgttaattg |
| 35221 | gctgtccttc ccccaccaga ctgtaagtgc ctcaagggca ggagtcctgt cttattcact |
| 35281 | ctgaatcctg aatcctcagc agttgctgca atgcaaagca cagagtaggt gcttaaggaa |
| 35341 | cacctgttga attaactgac actaagttta gtatatttaa gagctcagta atgggctggg |
| 35401 | cgcagtggct cacgcctgta atcccagcac tttgggaggc caaggtgggc agatcacctg |
| 35461 | aggtcaggag ttcaagacca gcctggctaa catggtgaaa gcccgtctct attaaaaata |
| 35521 | caaaaaatta gctgggtgtg gtggcaggtg cctgtaattc cagctactcg ggaggctgag |
| 35581 | gcaggagaaa cgcttgaacc caggaggcgg aggttgcagt gagctgagat agcaccactg |
| 35641 | cactccagcc tgggcgacaa gagcaaaact ctgtctcaaa aaaaaaaaaa aaaaaaaaag |
| 35701 | ctcagtaaca tgagacaaag gcatctacaa gacatggctt tggagacaaa gaataaattg |
| 35761 | ataaattgga aggaaaagaa tataacaatt cctcagtcct ttctggagtc cacattctag |
| 35821 | tggctttcag ctctacactg tatcaggcct cctacaaggc ttaccaggca gactgggcag |
| 35881 | aaattattga cccccattca acagctgggg aaggtcatgc ctcagagagg tgaacctact |
| 35941 | tcagctgaag ttacccagcc aggtggtggc aaagcatgtc caaagaaagt tagggaccag |
| 36001 | agacgtcaga tccaacccct ccctcttgca ggggatgggg taggggtggt aatctgcaga |
| 36061 | ctctgaacta agtgttctag ctcatttcaa actcagaata accatttgag gtaaacagta |
| 36121 | gccatagcca caaaaaaagt agacacatga tttgcccatc atccaccttc cccctctaac |
| 36181 | ccaattgtcc ctgggcagac cgtgcagagt accaccccat tcgtcctcaa ggactccctg |
| 36241 | cctggggtca actcatgaga aactgtccac ccgcagattg actgggacag aaaaaaccct |
| 36301 | agacttttaa tttccttatt tcacagttgg agacaatcca aagcttcgag agaaggaact |
| 36361 | agtctatata gcacatactg agacagggtc agaactccgg agtcagaact ttctgaagta |
| 36421 | tagcccacaa gtccccttga catgaacctt ttgagttggg aaagggctaa gaatgatgtg |
| 36481 | aacatgacct ggccatttgt tagctgtggt caggctgggc gaagagaggc agcccctctg |
| 36541 | gtagttctac tctagaactc attagtcccg ccagtcctca atggtttgtg cattggtagg |
| 36601 | tagcccatgg gaagtaggat gtgggttgct tactgcttag tgactgttta tgaagattgt |
| 36661 | tattgctact acagcagtat agtctcccag ctgaacaata acctagaaag tatggagagt |
| 36721 | ggccgggcgc agtggctcac ccttgtaatc ccagcacttt gggaggccga ggcaggcgaa |
| 36781 | tcacgaagtc aggagttcga gaccagcctg gccaacatgg tgaaaccccg tctctactaa |
| 36841 | aaatacaaaa aattagctgg gcatggtggt ggtgcctata atcccagcta ctcaaagggc |
| 36901 | tgaggcagga gaatcgcttg aacctgggat gcggaggttg cggtgagctg agatcatgcc |
| 36961 | attgcattcc agcctgggag acagtgcgag acttcgtcgc aaaaaaaaaa aaaagaaaga |
| 37021 | aagtatggag agtaggtgga gaggggctag cagagacata ggtagggcac cttaccacta |
| 37081 | agaaatagag caaagggtag gtgctttatg ttccagaagc atatagatcc aagaggaggg |
| 37141 | aagggacatt ttggctgaaa gcttggaact atatctagca tagataaatg tgattctcat |
| 37201 | aggtgcttgt atttgctatg ggaccctgga ccaatcattc agtctcttga agccttagcc |
| 37261 | tctcccataa gagggtggaa tgagctagtc tctagggatt cttcttttta taactctccc |
| 37321 | tctaagtggg ctttgcaggc agagggagtc attaactcct gctatggtct gagtgttccc |
| 37381 | cctcccgcat attccgatat tgactttttt tttttaagag tctcactctg tcttccaggc |
| 37441 | tggagtgcag tggcacgatc tcagctcact gcgacctcca actccctggt tcaagcagtt |
| 37501 | ctcctgcctc agcctcctga gtagctggga ctacaggcgc acaccaccat gcctggctaa |
| 37561 | tttttgtatt tttagtagag atggggtttc accatgttgg tcaggctggt ctggaactcc |
| 37621 | tgacctcatg atccgcctgc ctcggcctac taaagtgctg ggaatacagg cgtgagccac |
| 37681 | actgcgcctg gctatattga catcttaacc cccaagtgat gctattagga ggtgggaact |
| 37741 | tgggaggtga ttaggtcata aaggtggatg gagcactcat gaatggaata aatgccctta |
| 37801 | taaaaaaggc ccaagaaagg cccctcaccc catctaccat gtgagggtac agtgagaaga |
| 37861 | cagccatgta tgaggaagca ggtcctcacc tgatcccaaa tccttgatct tggacttccc |
| 37921 | agcctgagaa ctgtaagaaa taaatctgtt atttacatgc cacccagttt atagaatttt |
| 37981 | attatagcag cccaaacgga ttaagacaac tccatttcag gtaagcccaa caaacctagt |
| 38041 | atttgccagg ccaggtcctg ttgaggctta attgagaaat gtgtagcaaa ttgttagtag |
| 38101 | tacaggcccg agaccatgat ccttagaccg ctacacttgg cctgtggtca gctcttggga |
| 38161 | acttctattt cagaaggatt tccaccattc cccaaactaa taagagtagc tcattatgcc |
| 38221 | taaactggac aaacaatgca gtttctgttg actgcctaat tttcttctgg gagtttagaa |
| 38281 | ttctggtatg tgcttagcca aggatgccta tgtgaccagc ccctaacaaa aNcctggcat |
| 38341 | tgagtctcca gtgaacttcc ctggtaaaca acattttaca catgctgtta cagttcattg |
| 38401 | acaaaggaat gaagcacatc ctgtgtgctt aattcagctg ggggatgatt ctgggaatct |
| 38461 | cgtggctgtt ttcttccaga ctttgtccca tgcacctttt ccctttgctg attttgtaac |
| 38521 | cttgtgctgt aataaatcat agtgtcagtg tacttatatg ctgagttctg tgagtcctcc |
| 38581 | tagcaaatca ctgaacctag aggtgatctg gggaccccca acaggagtta agcctagatt |
| 38641 | tcacagagtc aatcataatc ttgtgccagt cccttaagag tatacagctg aaaccagtgc |
| 38701 | tatccaacag agatataatg tgagtcacat gtaattttac attttctagt ggctatattt |
| 38761 | taaaaaccaa aaagaaacag gcaaaactaa ttttaagcac atgttttctt caacccaata |
| 38821 | tattattatt cttttatttt tattttcgag acagagtctc actctgtcac ccaggctgga |
| 38881 | gtgcagtggt acaatcacca ctcactgcag cctcaatctc ctgggctcca gtgatttgcc |
| 38941 | cagctaattt tgaaaatttt tgtagagatg tggtctcact atgttgccca ggctggtctc |
| 39001 | aaactcctgg gctcaagcca tcctcttacc tcagcctccc aaagcgctgg gattacaggc |
| 39061 | gtgagccatt atgctcagct aacccactat attaaaaata tatttaaaat atttccatat |
| 39121 | gcaattttat taaattaaga tattttatat tcttttatca cactaagtct tcagaaaaca |
| 39181 | ctgtgtgttt tatactcaca gcacagatca gtttgtacta gccctactcc aagcgtgcaa |
| 39241 | cagccacaca cgcccatggc ctgcctgcta tattggctta gaggttaatg gcaagggctc |
| 39301 | tggagacagt atatgcggag gctaaagaca ctctgtctag gatgctgatt caaccatgtt |
| 39361 | gatttctgat taactccagt tccaggaaag ccgctaaggt tttcagttta tctactcttc |
| 39421 | cttgtgtaag agcatgtcct taacataaat attcccctta ggtgaaaaca aacttaatat |
| 39481 | tctcacactt cagttggcct acacatccct tcaataggga aatcgcccct tccctatgct |
| 39541 | atataagccc tgggctgagg aggggtaata gcttggggat ccactatctc acttcacagc |
| 39601 | cacctaagac ttctgtggct tctgtttcta agtccctaat aaatgtttct ttctgagaaa |
| 39661 | ctgggtatgt cagcttcttt cttcagcttc tcagcctcct ctgactggct ggcgcggggg |
| 39721 | tggggtaggt ttacagagat ctgctcacag cagaatagta tgtgtgggtc agaatcccgg |
| 39781 | ttctgtgact taccaactgc ataaccttaa gcaaagttgc ttaacctctc tatgtattag |
| 39841 | tttcttcatt tacaaaatgg ggataataat gttggctacc tcaagaggtt gcagagatta |
| 39901 | attaatctaa ggcagcacct ggtatagata ggatattgag tgaatgctgg ctgctactct |
| 39961 | ctttatgcta aagcttggcc catggagatc aacttgtcta acacctcgtt ccataaatga |
| 40021 | gaaaactgca gcccagagtt tggaaggcac tagtttcaca agcaaaaact acagccaccc |
| 40081 | acctctcctc ttgatgaact ccctgtcttc tggctggccc tgagatcaaa tttgccaatt |
| 40141 | atttaggaaa ttggcagctc cagggcccca gctcggttct gcaatttctc tcgattgcaa |
| 40201 | aacaagggct actgtttgaa gttggcactg aagtgacgca ggctgggcct cacttctgct |
| 40261 | tccacctagg ctcatcagtt agagtggctg atcccatctt tgagttcaga acctgaaatg |
| 40321 | tttactaacc aataataagg ctggctttcg ttgttttaac tttttacaat cctttgagtt |
| 40381 | tattaatttg tgttctatct tccttgaaga cacagtgcat cagcactttt tgtaaaacct |
| 40441 | ctttttaagt catcttcggt ctctgttcac ctaccaagta ggtaagtagt gagctcccta |
| 40501 | tagaggtctc taaaaaatag gatgaagagg ttccgagtgc tcacttgtct ggaatgcgga |
| 40561 | agacgtgttc tcagactgga agcaaaacta gcttatgatt cttctacagc ttctccttcc |
| 40621 | atccctccct tcccattttg gccaatgcaa atcatctact ctcctctgac aagcctactc |
| 40681 | atcataaccc gcacgtcttg aatcttccta gaagatgaaa tcagaccagt cttgcgggct |
| 40741 | ctacatggtt acaacttgct aacttgaagc tctcattatt ctatatgcca aatattgttt |
| 40801 | tccttgacat taatttattt gatagatatg tatcaagcaa ctactatgta ttaagctctg |
| 40861 | tgctggtgct ggtgYctttt tggatcttat aatttagtgc agaaaataga caaacaagaa |
| 40921 | atgatgataa agtacaacaa agggtcgatg tacaaagtgt caaatactct aggaactaca |
| 40981 | ggaaaacaag ggaaccatca cgaaacccct gtcttaagac gtgacattag tggagatatg |
| 41041 | aaaaatgagt aggatagtac cagttaaagg gagacaacct ctgttccaag cacagggaac |
| 41101 | agcagagatg aaacctggaa gtgagggaga atggcagact gcaagaacgg aaaaatgtcc |
| 41161 | agtgtagcgg aggcagagag aatatggaga gtgtggcaaa agaggaagaa gcagatggat |
| 41221 | tttgagggca ctttctgaag tgctaaggaa tttagacttt aacctgaggg caatcagaag |
| 41281 | cagcctggtg gagaacgata tatttcaaaa catagggtcc ttgaaaacaa gtcaaccaaa |
| 41341 | tgtatttaaa aggctgctga tgaaaagatt gggaagtatt gttttcattc tcattgtttt |
| 41401 | cactgtcatg ctctttgtgc caatggttag acctcctttg aaagctcact gcagcatgaa |
| 41461 | agcccttctt aatgccaggg caggctcccc aagcctcccc accccactgt ccatgtcatc |
| 41521 | agttttgtac tcctgttttg tatggctcag gctttcctta tactcttagg aatgaaaagc |
| 41581 | cacagacacc accactgaga gactgtcaaa gatccttgtt aaaYaggtgg acaactgaga |
| 41641 | tcagagaagg aaagtaattt tattactttc Ygtagcaatc caggggtagc atctcaggtc |
| 41701 | tctggctcct tcttgtctcc cctaaagaaa tagaccacca gggctgggat taaggtgaga |
| 41761 | caagtgagac atagagggtg caaaatgtag agagatgccc gctgccaggt gccagcctgc |
| 41821 | acttRtgtga ccctaggtgc cttcccttcc ctcaccctgc tggtatctgc tggaaacacc |
| 41881 | tttgagagca gggactttgg cttttagaca actctttagc atctctcatg gtgttaacat |
| 41941 | gttgctaggt gcctattttg agggtcacta gatgcctact gatactaatg aacaatggat |
| 42001 | ttctgacaaa ctgatgtgca cacagatgtt tatattttag ggtaaatacg aaccttaaaa |
| 42061 | gacaacagaa caacatggag agggcctgag actgagtcta gagacctgga ttctggttcc |
| 42121 | ctccctgcca tcagttggtt atgggaactt ggataaattg ctcctcatct ctaggcttca |
| 42181 | gggaaatgag gtggttgaat aaaacgtatt gagcacttac tgtgtgtcag ccattgttca |
| 42241 | tatgctccca ggctttaact cagctaaaga cttcctggcc tctgtggttc cttccacttc |
| 42301 | aacaggctgt ggtttgaggt gggaagactt cgtagagaat gcaaaacttg agagacatct |
| 42361 | tccaaattcc cagggggatg ttaatacctt taatcatagt aaaggtatgg tttctcaagg |
| 42421 | catttcttgg tcagaagaaa gttccagaag gcacttaatc ctaggactca cactaccaag |
| 42481 | gctacagcca ggccaactgc ctcacaataa ccttagaaca tgtctgaaga cagattttca |
| 42541 | gcttctgccc ctagagattc taatgcagtg attccaaagt ggggtccagg aatctgtatt |
| 42601 | aaaggcttcc cagttgactc tggtggtcag agagatttga gactgattcc atcacattcc |
| 42661 | ccatctgtga acgggatcat acctagacag cccagcccta gaactgcctg gtctgatcct |
| 42721 | aagtgttcca tggagatttt gtttttttaa tgtaatgaat gattaagtca gcaaatactt |
| 42781 | tacatctctt gtgtgctagg ccctgtgcca ggtggtgatt atgggcccta ttctcaaagg |
| 42841 | actcctggtc tccgtctgtc acttacttta gaaacaagtc atctagccag agccagaagg |
| 42901 | gcactggaga tcactaatcM atcccatcat tacaYagctg aggaaactga ggcccagaga |
| 42961 | gtaactgcaa catgttaaag actacacagg gagcaaattt Wttttctgct tYtaagccag |
| 43021 | gtggtttggc cacagatagg ccaagaagaa tacgaacaga caagccttgc tgggtttccc |
| 43081 | actcagttta ttactattag accattccct ttttccccac tcacatttct gcgcatttcc |
| 43141 | atggtcttca tagaatctaa gcctaaaaat agagttttcc ttgagtcttt gggtcttcat |
| 43201 | ttYtgaagac tctcatgtca cagaaaactt taattaaata aatcgttatg cttttctctt |
| 43261 | gctaaccttt ttctacaggc atgtcagctg tgaYYcttat gatggggagg aaaggcaccc |
| 43321 | caccttctct gtccctacag tccYaatgcc ctacccagaa tggggttttc aaaatcccag |
| 43381 | gacacccact ctttgtttct taagcactaa acctatatat agtaaatgtt tctggaagtt |
| 43441 | taacttcagc tcttgctgaa attttaaacc ctcaatttcc agaggaccat cgccaggtgt |
| 43501 | ttttaaaaYc tcgataggaa taagaaatgc ttcccagaat ggagacttSc atcagttctW |
| 43561 | gggagctatt agcccccWtg ccctggctgt aatggagacc gctgtgtctg cctcctttcg |
| 43621 | cactttggag ccaaaagagR aagggaccgc ctcccacgtc cacagggacc tgacttccac |
| 43681 | ctctctgccc agatttgctt atgtcactgt cgccccggga cggggaggtg gggagctgag |
| 43741 | ggcaagtcgc gcccgcccct gaaatcccag ccgcctagcg attggctgca aSggtcYcgg |
| 43801 | cttggccgcg gattggtcac acccgagggc ttgaaaggtg gctgggagcg ccggacacct |
| 43861 | cagacggacg gtggccaggg atcaggcagc ggctcaggcg accctgagtg tgcccccacc |
| 43921 | ccgccatggc ccggctgctg caggcgtcct gcctgctttc cctSctcctg gccggcttcg |
| 43981 | tctcgcagag ccggggacaa gagaagtcga aggtgagtga gcctccgggc cgggggccgg |
| 44041 | gagaaaaaac ctagcccctc ggtgtccagc gctcagtgca atgcacccct tttcccaggc |
| 44101 | tccccgccag atgggcaatc cccaggtgcg agagacctcc tgaacccctt ttgccgcccc |
| 44161 | ctccgccgcc gggaccccgc ccccgaccgt cgtcgtctcg tagttccatc tgttggagag |
| 44221 | ccgagacctg gtgcttcagg cgggcagaat gactaaggga ggaaggtctc tctccccgag |
| 44281 | ctcgcacttt ctccccRctg ccacctcgag Rgtcgccttg ctacatctat gtcacctccg |
| 44341 | tgcctgagct gctccccttd agtagggcct aagagggagg gcgtcacaga aaaaaatgcc |
| 44401 | cagcgtctgg gtgggctgtg caagttccca gggagagaga caaggagaaa ggagagaggc |
| 44461 | agctgggtgg tcctggtcct gaagaactgc tgtggggggc tcttctaccc caagaatgat |
| 44521 | acaggcaggt agaaatggcc acttggattc agggcattgg tagaagaggc agagatctgt |
| 44581 | gctagaaRga gctctggagt gacagtgcag aacactcagg accctgagtc cttacctgct |
| 44641 | cctggacccc cgtttctcca tctgcagggg agagtttggg ggtgggagtg catggggcac |
| 44701 | aatgaagctg agggcctcag aggggatgct tggagaagct gaaaaatcca caagctgttc |
| 44761 | tctgaaattt cctcYcctgg ggcagcctca ctacccagga cccactgtcc tcattagcct |
| 44821 | gaggaaacct ggtagttgga gaaagctggg gactgctaat tcagaagaag aggtagaatt |
| 44881 | ctctagagcc tcagtctatt tccagcaggt gtgtggccca Yacctggagt ggggatactg |
| 44941 | aggccaggag tgggatgacc cagcatgctt atctcctttg acagtatgta ctggggtggg |
| 45001 | ctctgYagac ctctcctcta cccagggagt gggagtgggt ggggaaggag gcagaagcca |
| 45061 | ggtgtaggga tagactcaaa gcgagcgatt tctgtctYca acttcccaac atgcataact |
| 45121 | cacatttttg caagctctga gcttttgttt ctaaaatcct ctgggtcttc actttaatta |
| 45181 | agttSttatg agtctaggat tccgtttcca aaattctgtc cacttctcag gaagcccatt |
| 45241 | ggaagcacag ggttccttcc atgttactca ccccttttcg gtcctcagcc taaatccgat |
| 45301 | taatcagcct ctccttggag tcagtctaaa tggatgtgaa gccacttcgt ccaagtcccc |
| 45361 | tcctttttat ccatccctgt tcataatccc caactcagaa ggcattttcc aggtcaggaa |
| 45421 | tgggatcgat tttcaacatt gaggtttggg gtgggccata gacaggccag caccRtggag |
| 45481 | tcagtcccaa ccttcagttt tggaaRggtc aaagaagata caattgggcc ctgcctcaag |
| 45541 | aaSgctagtg gggagaacgt ggactccatg aataactcat catagctgtg tcttctctgc |
| 45601 | Mgcactctgg ggaaggataa atggactcag agagaatagg atggggatgg tYtagaaaag |
| 45661 | ttttagggtg aggtgtgatc atatcacata caaccagtca ggacttgctg gctaatgaaa |
| 45721 | gcctgggcat gcccatctct gctcccaagt tcttaNagtt cagatgtctt agctctgggt |
| 45781 | taggacccaa ccactcacct tacccccaac cccagggtaa agagaYaggg ttatggggca |
| 45841 | tgtggagaat ggaatggacg aaaggtgact cagccccaga gaggtgtttt cctcctacca |
| 45901 | attgaaaggg agatgctagg gaagagacca ggtcctcctc ttcttgctgc gtcacctccc |
| 45961 | cagtccaact tcaSgctgcc cacgctcctt gtctctacaa aaggactgca gaagccggaa |
| 46021 | agagtgacaa ctgctgacgt gcaYgggatc ctagtaactg ctggtttcta ggtgactaaa |
| 46081 | cctggcagag agaatcatgg aaccacaggc tgacagagtt aaagggccct taagacagcc |
| 46141 | ctgaacctga ctccatctta caatggagga gctggagccc cagcaaaggg ggtcacatgS |
| 46201 | catcagagac acagcaaRgc Ycagcactca ggtctcctga ctcccagacc acttctcaca |
| 46261 | gcatcatatt gataggagtR ataagacggg agaggagaaa gacacaagac cagcagcccc |
| 46321 | agccagccag agtcctgNct gagtcccatc atccaactct caagccattg gaaccctctt |
| 46381 | ctcttgcccg gctgtgggag gattcaggca ccctcatggc ctctcccctt atctcctctg |
| 46441 | gccactgttt cagatgcggt gctctgggat tccacgtgcc cagagaaggg aagggggctc |
| 46501 | atagaggact gagttgcagt gatcagtggt gagcacagcc gtggctctcc acccatgggc |
| 46561 | caccttctga cccccactat ccYttgacat gagatttcgt tccaagagac tccaggcctg |
| 46621 | ttttgctctg ccagcagatt cccacaaccR ttttgggcag cagcatgctt atctccctgg |
| 46681 | acatatgccc agacagtgta agtgacttgc tcagggccac Rcagccagca aaaaatagaa |
| 46741 | cctaatgaaa tagggttctc cttgccacac ccccaagctg aaactgtgcc cagaagctgg |
| 46801 | tccttctStt tacaagttcc cattattagg acaacctgta atcttttttt cctccatgtc |
| 46861 | tcagagttca tttttggatc atggcagagt taaaaaagtg gagaggctat agacatgcct |
| 46921 | ggctttctgg ccactagatc tttgtggcgg tctagggtgt atttgttgtg ccccagtttg |
| 46981 | tctgcatttt aaggcggaac tggccctggc tgggaacgag gaggtccagg agcagtagga |
| 47041 | atgttgatct taggtcttgg gcctgcctcc agcacccacc aaaagtcacc ggatgtggaa |
| 47101 | tattagactc atgtaaagca ggaagtttgc accagagcca agaagagagc ttgcagctgc |
| 47161 | cagggaatgg cctggcggag gaagcgcagc ctcgcccagc tcagcagacc tgcttcatga |
| 47221 | cagaggcagc tcttctgcac tMtactgggg ccacgccctg ccagcatata tcactccctt |
| 47281 | cacctggtca ggcccaggta ccttgcctca acagcaagtt ctccagaatt gaggggaaga |
| 47341 | gagggagtga atttgcttct ctaactccag ccacctgtct ctgccctctc ctggtctata |
| 47401 | atgctacttt gcattccatt tgtttattgt ctgcccacct ctcaaaggat atcagggcca |
| 47461 | caaaggacct taaagaccac cttatacaat ggtttcaagg tttctccttt cYcatcacga |
| 47521 | ccagcccacc tgccaaattg tgtcaatgat gcaatgacat cctttcaaga atagtctcag |
| 47581 | aattttgttg tctgtgtcaY gggcagagta ggctcatagg ccacatccta ctagccagca |
| 47641 | gtgaccccaa tcccctctct cccactgatc atgatcctat caagccatca tggttgacct |
| 47701 | ggttgRagag ttggttcagt tcaaggttct cattttataa atgaggaaac tcaggtccga |
| 47761 | agaaaagaaa gggccagtcc aagaccacac agggagttaa tggcaRaggg acatggagcc |
| 47821 | caggtctccc agcccacagt ccactgcact ctgtccctcc ttgtcctgga ggctgactgt |
| 47881 | gagcagcagc tggggaggga ggaaggcata agggtcatca ccagcctgct cagacccgga |
| 47941 | acattaacac atgcaggtac tgttgctttt ccctaccKca ccacagccct ttcccccatg |
| 48001 | cctggctctc agtcctgctt YcctRtttgg taacaacagc ttcatctttc ctgtcctgca |
| 48061 | gattgggagc ctctgaatca ccatgtccat acKgcctctt cctcctttct gttcagtttt |
| 48121 | tccttcacac tggcctagtt caggttctca tcctcaattg cctggattat tccaacagcc |
| 48181 | acccaaccag tctccatcag cccgtctagg aacccagtca tcagctttag ggcagtctcc |
| 48241 | ctgagagccc agtaaatgtt ctgaaattga catgtgatgt ggaagtaatt agcaaaataa |
| 48301 | gtcattctcc agggttccat agcactcact tattaagtgc ttaatattaa taagtgctta |
| 48361 | ttaggtgctt aatattaata agtgcttttt aagcacttaa tattaagtac ccRaaaagca |
| 48421 | cttattaagt gctttcataa tcattatctt aaacaaggaa ttaggagacg tcagtgctag |
| 48481 | tccagttcca attctcggtg attactttga gaaagtcact ctctctgttg ggatctttca |
| 48541 | ttcttttgaa aacggagatg tccaagttcc tttccagctc taactgctcc ttttatggcc |
| 48601 | tgtgttccag atggactgcc atggtggcat aagtggcacc atttacgagt acggagccct |
| 48661 | caccattgat ggggaggagt acatcccctt caagcagtat gctggcaaat acgtcctctt |
| 48721 | tgtcaacgtg gccagctact gaggcctgac gggccagtac attggtaaga gcccaccctt |
| 48781 | cctccctgct ttatttgggg ctgtatggca tatttcaatc acaggagctt ttctggtgca |
| 48841 | tgggggaaag ggtgatggca atcacgagag tccaagcccc ttttctcagc tccactgtgt |
| 48901 | tccgtggttt tgtgaagatg attatataag ccYgaggtct gattgccttt ggacatgttc |
| 48961 | taggagattc ctagttatcc ttcttcatct ctgggcacct caacaaccct aaaggcagag |
| 49021 | ggatagagat taggtttgtg cttagagctc cctttggctg gagcatgaga tggtaactga |
| 49081 | agctccatct tgctgaSaat atctYatttt ccctcagccc catctgcttt ggtgcttctc |
| 49141 | ttggcagctc tctggaaagc aggcaattgc ctcgaggccc cagagtgtat gcgtggtgta |
| 49201 | gcgggatgaa ggagcaggct acacttctaa tgcccattct aaaataaact ttgaagatga |
| 49261 | tctagtttag ccttcccctc ttctcaatct acacatggga aaccaagact cagagcgaag |
| 49321 | atacaactta aacagggtca caaagctatt agcggtggaa tggggcattt ggcgcaggtt |
| 49381 | tcatgatctc ctgatatgct tactcctttc tccctagctg gggtagaata cagaggctag |
| 49441 | gggaacaggc aaccaaaggc aagcgacagc acccagggga aaacgctttg gggctgagca |
| 49501 | gtctactggc agggggtcag ggaggcaagg gcaatttgac cctccatgct ctgcctggca |
| 49561 | atcacaggcR aattcccgga agtgaaagga Yggcggtcac gtggcccagt ggctgtgagt |
| 49621 | gctttttctg ggatgcttct ctctagcctt ggaacagagc agagtgcgtt tgggcaagag |
| 49681 | atgggctggg tgctgctgca aaaSWgacca ggggacactg aggaaactgg ggagctgggc |
| 49741 | cagggcctca tctgtgttcc tgatttgcca aagattatgg gagggttgta acctcacYcc |
| 49801 | aaagaagttc tctgccttgt atccagggat ggatagttct tgctccagaa aactcaatcc |
| 49861 | tgagggttct gaatgagtgc tctagctggc ccaggtggag ggccttggca atgggcaggt |
| 49921 | gactgacagc tgacttgagg aagggtatta ttcttgtcct ccaaacctcc aatccatcca |
| 49981 | gattagagtc aagaggatag gacctgagtc ccagcattgc tgtgaactca ctggtgatcc |
| 50041 | tgcgcaagtc cctccctttc tcgggcctca gtagttccag cRgcacagcg ggtgagccgg |
| 50101 | gggagtggtg tggatgagac agggctcctc gccaggatac tcccacRtct gctctttctc |
| 50161 | tttggcccag aactgaatgc actacaggaa gagcttgcac cattcggtct ggtcattctg |
| 50221 | ggctttccct gcaaccaatt tggaaaacag gaaccaggag agaactcaga gatccttcct |
| 50281 | accctcaagt gagtactcac tcagcatcct gagaaagctc ctctcacatg gcccacatct |
| 50341 | tgttatcaac cccaaWtcat ggtggacatt tatcggccac caagaactac tctcctcttc |
| 50401 | taggatcccc agtggaatga gggaagggaa gggacaagag agggagaagg acagggacaa |
| 50461 | ctggttgtga tgtgcatccg cagggagcac caaggttgag ggacactgaa aagggaccag |
| 50521 | gctagaaagg aagaccgtgg actcacatta tgcctatgcc cactgcacat tcactggctc |
| 50581 | ctgctgccca ctgcagaata aatccagact cccaacaccc tctcccctgt tctgtccctt |
| 50641 | cctctcattt ctgagccctg tgcccacctc cttgggaccc acctaagaac atttctcaac |
| 50701 | aggtatgtcc gaccaggtgg aggcYttgtc cctaatttcc agctctttga gaaaggggat |
| 50761 | gtcaatggag agaaagagca gaaattctac actttcctaa aggtaagtga gctgccacct |
| 50821 | gtgctggctg gggctgcagc ccctcctggc tccagcccac agcgtcaggg cccatgccac |
| 50881 | ctcccctgct cctgggctct tggggaattt cttggcacct gactattgtt ccaactagag |
| 50941 | ggctctgcag accctgacta gggtctcMtt ggcccatttt acagaaaggc ccagaaggac |
| 51001 | ccagagtgaa catactaagg gtctcacaMt cttctagagc cacagctggc gctggcagtc |
| 51061 | ttctaactcc caaactgggg ctcttYtctc agggccaggc tattccccag gaaggcctgg |
| 51121 | gaaggaagag ggtcaggggg cctcaagcaa ggttgacact cctctYatcc ctgctctaga |
| 51181 | actcctgtcc tcccacctcg gagctcctgg gtacatctga ccgcctcttc tgggaaccca |
| 51241 | tgaaggttca cgacatccgc tggaactttg agaagttcct ggtggggcca gatggtatac |
| 51301 | ccatcatgcg ctggcaccac cggaccacgg tcagcaacgt caagatggac atcctgtcct |
| 51361 | acatgaggcg gcaggcagcc ctgggggtca agaggaagta actgaaggcc gtctcatccc |
| 51421 | atgtccacca tgtaggggag ggactttgtt caggaagaaa tccgtgtctc caaccacact |
| 51481 | atctacccat caYagacccc tttcctatca ctcaaggccc cagcctggca caaatggaKg |
| 51541 | catacagttc tgtgtactgc cRggcatgtg ggtgtgggtg catgtgggtg tttacacaca |
| 51601 | tgcctacagg tatgcgtgat tgtgtgtgtg tgcatgggtg tacaRccaYR tgtctRYcta |
| 51661 | tgtgtctttc tgggaatgtg taccatctgt gtgcctgcag ctgtgtagtg ctggacagtg |
| 51721 | acaacccttt ctctccagtt ctccactcca atgataatag ttcacttaca cctaaaccca |
| 51781 | aaggaaaaac cagctctagg tccaattgtt ctgctctaac tgatacctca accttggggc |
| 51841 | cagcatctcc cactgcctcc aaatattagt aactatgact gacgtcccca gaagtttctg |
| 51901 | ggtctaccac actccccaac cccccactcc tacttcctga agggccctcc caaggctaca |
| 51961 | tccccacccc acagttctcc ctgagagaga tcaacctccc tgagatcaRc caaggcagat |
| 52021 | gtgacagcaa gggccacgga ccccatggca ggggtggcgt cttcatgagg gaggggccca |
| 52081 | aagcccttgt gggcggacct cccctgagcc tgtctgaggg gccagccctt agtgcattca |
| 52141 | ggMtaaggcc cctgggcagg gatgccaccc ctgctccttc ggaggacgtg ccctcacccc |
| 52201 | tcactggtcc actggcttga gactcacccc gtctgcccag taaaagcctt tctgcagcag |
| 52261 | ctgagcctac tgtgtgtggt gcttcttcaa tggtggccgc ccccgcctgg gtgggaagtg |
| 52321 | aggaggagaa ggtggggcag ggtaaaggSg ggaggatgag aaaagaacaa atatgacaac |
| 52381 | aaaaactttt tacagtgggt gcaaaggtaa ttgcggtttt Ygccatatgg caaaatccaa |
| 52441 | ggctacctcc ccaccccaca attctccctg agatcagcca aggcagactg caagggccat |
| 52501 | gtagccctat taggggtggc atcttcatga gRgaggcccc cctgccacaa aaaaggggca |
| 52561 | aaaattgcaa ttactttttc aacaacctga tagttagctg cagtggcgca ctccttcagc |
| 52621 | tacttgggag actgatgtgg gaggctccct tgagcccggg agtttgaggc tatagtgtgc |
| 52681 | cRtgactgag tctgtgaaga gccactgcac tccagcctgg gcaacacagt cagactgtct |
| 52741 | cttaaaaaaa aaaaaaaaaa aagagagaaa ttagacaatg ctttgagtcc ctgctttttg |
| 52801 | gctcctacct tggcccactc tgcttctctc actcagcatt tggagtttat aaaaccagtt |
| 52861 | ctgatctact ctcattccct cccacagttc tccaagtcag ctgaggttaa gatgaggtgt |
| 52921 | tctgtttgac ggatgaggaa actgaggctc acagaagggc cacgccttgc cccaggtctc |
| 52981 | ctgatgagtg aggggcagga ctggaattta aacccaagtc ccctgagtcc tggcctaggc |
| 53041 | tccttccact gccccggYtg ccctaccctc agggaggccc tgatacgtca ccattactca |
| 53101 | agagtaatgg tgaccatctc agggaaagga agaaaagggg aacagaagga gggacccagg |
| 53161 | aaagcagggg gttgacaggg gaacatYagc gcataaatga acatgagtca gaatgttgat |
| 53221 | cttcttcaac ttggatttat gtccatgatt cgtgcaaata gctatccagg gatggacagc |
| 53281 | caccctaatc tgggcttctg gcaccacagg cctccagcta tggggtccag ggtctgaacc |
| 53341 | tcagggcctg gcagcttcag gctggcgccc ctcggcRgac gtggctggca tggccttctc |
| 53401 | ccatctgtga tggcttcagc aaggctactg tgagtggtgg tggagagggc cccagagcca |
| 53461 | gaatatccat cattctcttc tgttctggag acaaacccac acttcccaca gctcctcaca |
| 53521 | gaggggtagg ggtgtgcgtg gggcaggtcc tgctacagag cttgaagcaa aaattaaaca |
| 53581 | cacacacaaa ttggtcatgg caactagagg gcctgaaacc acttccggga ggttttgaag |
| 53641 | gaagggggtc ttggctgcct cccactctta ggattgctgc tcctggaggc ttctgcaaYg |
| 53701 | gatggtgggt caaagccgga tgaggcctct ctccactcag cagcagggaa ggagtttttc |
| 53761 | ccagtcactc ccagcagagt acaaatgaaa gccttctggg tggagcctcc ccagtcctgt |
| 53821 | aaacagctca gttcagggac tggtRtacaa gctggccacc catctcagcc tctcatccag |
| 53881 | ctgaggctct ggccacaccg tgcaagtggc ttctagtttY ttggcaatMt gagatcagct |
| 53941 | ggctctgcaa gatgaaggtg gagccaaatg acacaatctg gtctcactga ggcccctcac |
| 54001 | ggtcattttt tggagactct aaataaacaa aaattttgag gacttcatga ttatgtggta |
| 54061 | gagcgagttt caaagtctct tatggaatgc agtgcaggaa cagtgaaaat aacagctagg |
| 54121 | gttactgggt gctaccaagt gccagggcta agtaccttaa gtacgctggt catgtaatct |
| 54181 | tcacccagcc ctatgaagaa ggcaggttat tatcccattg taaaagaggg aactgggaca |
| 54241 | ctaagaggtt ttaaatgact tgctcatagt tccaggtcca ggaaatagtg gggctaggat |
| 54301 | ttgaacccaa tctcaaatcc tggagcagtt ctggggaggg agcccctccc attataccaa |
| 54361 | tctctaccac tcatcaaagc agcctccaag gggctccact gaacttgtag ggttcttcag |
| 54421 | ggaaaaacag aaaatgactg agcagcccaa tcccttcttc caatcaccaa atcctccaaa |
| 54481 | ctgcaacccc tccaaggtgt ccgtcccatg gcctctactc acacacctgc aaagggacac |
| 54541 | tcgccacctc ccagagctgg ctccatttct gcacaactgt gtcccttaga aagaaagttc |
| 54601 | ctcttcatgt tgcgatggac ttggcctccc tgtcattccc acccactagt tataattctg |
| 54661 | ttcccagaag tcccacaggc gaggtggtgc cttgtgcccc atgagagctc tacagggagc |
| 54721 | aatggcagtc gctaaggtcc caatcttttc acgagtggac acagctccag gctcttcagc |
| 54781 | ctttcatggt ggacaccatg gtcaggggag cttttcaaac agaaatatgg tcatgccact |
| 54841 | ctgctttaaa aatgtccagt gactacacat tatgctgagg ataagaccca ggcctcccac |
| 54901 | catgctccgc aggcctgcac ggtcaggacc cggctacgtc ctgaaccctg cgttgcccct |
| 54961 | ctctttccct accttgttct tggctccagc cacgctggtc ttccctccga tcctcaaatc |
| 55021 | agctccccta cttcccctcc tcccaccacg gcctctgcat ttgtcatgtt ctctgcaaaa |
| 55081 | gcatcctcca cccctacctc tccacctagc tagtgtcact cattctgaga gtctcaactc |
| 55141 | aaatgctgcc cccccggtct aggtcggctt cctctgctat ctgctcccac agaaaccggc |
| 55201 | tcttcttcct ttagggcact tgagtgcagc ctgtttgtgt ggtgtttgtt tattcatttc |
| 55261 | tgcctccctc attagacatt acgctgtata agggcaagga ctgtatctgc ttctgttctc |
| 55321 | taggcatcca tagcacctag cacaatgcct ggcattagat aatgcactag aatgaatggt |
| 55381 | ttcagccccc ctcactctgt ggttgccatt actctccatt caacactgtt ttggaaagct |
| 55441 | gggcctctcc tccaccaaga gcagaaacta acRacatcac ccccaaactc aggcaatgct |
| 55501 | ggcagataag atattatctc ccccagggag gaccaagact cagtattagg ggctcacctg |
| 55561 | gttctgtagg cctggctgtg ggagggtcca tcacttggga gctctgattt ggatttcgaa |
| 55621 | cccctccaca gggtagacgc caggtgtatt ccggctgcga caaaactggt gcttaataat |
| 55681 | caataatcaY acccaaaaat tacaatagca cctactgtgc caggacaata ctaaatctgt |
| 55741 | attagtcaag tataattgaa tctttataac aacccaggct ggcactccca tttcacaggt |
| 55801 | aaggaatctg aggctcagag gggataactt gctgaaggtc atctagcaaR gagaatggct |
| 55861 | aagcttggat ttgaatccag gtctttgtaa atccagagcc aatctcttaa gcgccatact |
| 55921 | atatactctg gtgtttaaag atgtgtacca agaagggtca tatgggagaa gctgaaaaag |
| 55981 | aggccagcca ggaggtttct gtaatggtcc aggggagagc tgggatgcct aaggtggggc |
| 56041 | tgtggcagag gactggggaa aatgggacaa gtgacagcag atctttggaa gaagaatgat |
| 56101 | aagaagggaa aagaggccag gcgcagtggc tcacgcctgt aatcctagca ctttgggagg |
| 56161 | ccaaggcggg tggatcacct gaggtcagga gttcaagacc agcctggcca atatggagaa |
| 56221 | accccgtctc taccaaaaat acaaaaaatt accgggcgtg gtggtgcatg cctgtaatcc |
| 56281 | cagctacttg ggaggctaag gcaggagaat agcttgaacc tgggaggcag aggttgcaat |
| 56341 | gagccgagat cgtatcactg cactccagca tgggcaaaaa agagtgaaat tccatctcaa |
| 56401 | aaaaaaaaaa aaaaaaggaa aagaaaataa aaaaagagag gagagaagaa gaggagggga |
| 56461 | ggggagagga gcggagggga ggggagggga gaggaaagga aaggaaagag agagaaacat |
| 56521 | ggtgaggctc tggaacctag ttacacgtgg aagttataag aaaaagcaaa cccaaactct |
| 56581 | gatacccatg gcctggggcc tggagggagt cctccttccc accctgcact cttactcagc |
| 56641 | cttctccgtc ctagaccagt gaccctgaac agctgtgggg cagccctgcg ggactgtggc |
| 56701 | cagatccctg cccctgggta gcagcccaga gccaggccct ggacacccac ctgccaccac |
| 56761 | Rgtgctgttt agttcagaag gatgcagttt ctgaacccct cacttaacct tcctctctgg |
| 56821 | aaggtgtctg gggcagcctS tgtgtgtgcc ctggagcaag ggagggacac agactcacca |
| 56881 | gatggaagag gcgcgagttg gggagtgggg gcgggtgctc catggccatg gggggagggg |
| 56941 | ggtagcggat ctgggaccag tcctcgaagc catggtgtgg caccatggct ggcatgggcR |
| 57001 | ggtaggcgta ggggtaggcc ccgcagagat gttctgggtg gggctccaca tggtaacgct |
| 57061 | ctcctgaggc ctgcaagaaa ggctgacgtc tggctttggc ctgcaacagg ctgcaMagga |
| 57121 | cacccatctc atcaccctct aaatagaatg ttagctctct gaaggctagc aggctgggta |
| 57181 | ccaaaggctg gtatgtggtc atgaaaggct gctacatggg tacagaaatc tggatacatg |
| 57241 | ggcatagaag gctggatata tgagcatgga aggctgcatg ggcatggaag gctgggtaca |
| 57301 | catgcatgga aggttgtgac atgggcacag aaggctggta catgggcacg gaaggctggt |
| 57361 | acatgggcat ggaaggctag tacatgggca cggaaggctg gtacatgggc acggaaggct |
| 57421 | ggtacatggg cacagaaggc tagtacatgg gcatggaaag ctggtacatg ggcatggaag |
| 57481 | gctggtacat ggacacggaa ggctggtaca tgggcacgga aagctagtac atgggcacag |
| 57541 | aaggctgcta catgggcagt gaaggctggt acatgggcac agaaggctag gtacatgggc |
| 57601 | agtgaaggct ggtacataga cacggaaggc tggtacatgg gcacggaagg ctagaacatg |
| 57661 | ggcacagaag gctggtacat gggcacggaa ggctgggtac atgggcacgg aaggctgggc |
| 57721 | acatgggcat ggaaggctgg gcacatgggc acagaaggct catacatggg cacggaaggc |
| 57781 | tgggtaaatg gacacagaag gctgggcaca tgggcacgga aggctgatac atggaaacgg |
| 57841 | aaggctgagc acatgggcat ggaaggctgg tacatggaca cggaaggctg gtacatgggc |
| 57901 | atggaaggct gggcatgggc acaaaaggct gggcatgggc acaaaaggct ggtacatggg |
| 57961 | cacggagggc tggaacatgg gcatagaaag ttggatacat gggcacggaa ggctcataca |
| 58021 | cggccaaaga aggctagtca catgggcacg gaaggttgct acctggacac agaatgtcaa |
| 58081 | cagtgatgac agctaataca ttgtgggcac tgcctctgta ccaatactgc tctaaggctt |
| 58141 | ttcctgtatt actcatcagt tagttagcag aggatgtccc cacgcccgag cacacactgt |
| 58201 | gcatccatca ggctagctcc atgaggaagg taagaggatg tcagtgctgc tacctccctc |
| 58261 | aagcctcctc acctttgctt tcctcttctg ctgtctgagg gcttctcttg ccttctcctc |
| 58321 | atctttgaat gcttttagct gagagaagaa gggagggaga aaagactgtc ggcacaggtg |
| 58381 | gtttctggcc aggcctggat ccagggccta accagccaMR aggactgacc ggctgatgct |
| 58441 | tccctctggg ccaccatgcg tgggccagcc ccgggaaagg gccctgaggg cggagcacag |
| 58501 | cccaggctgt tgctgcaggc cacctgcttg tcacagtggt ctgtgcacag gtcaagaggg |
| 58561 | ttcacgtgaa cctcgctgct tctctgctaa catcttttag ttccactctg ggagagtaaa |
| 58621 | gcccagaact gactgtggaa agtcagaggt agagatatct tagagtaaaa ctcgtaattg |
| 58681 | tacaaagaaa gagtcagaga ggggaagggc cttgccgagc tcaaatatga ttgcagagct |
| 58741 | ggagagaagc agtgagggtc ctgacatcca gcctcaccag ggccctccaa tccatgcccc |
| 58801 | ctctcccSac gaggacaggc cagttgccct tcctgggtcc agtcactctt acctgggcat |
| 58861 | ttgacagggt gacctgggcc tgcagcttct ccacttgctt cttcaRctct tccttctcct |
| 58921 | cattcatgcg ctcacgatca ctgcgctccc tctggaagtc ctcctcgaag atcttcacct |
| 58981 | ggtgtggagg gagggtgaag agagggaggg ggatggtcct gagtggggat ttctcctcag |
| 59041 | gcccagaYtc ccatgcctcc tgctggggtc acagatggca gagctggggg tcgccatggg |
| 59101 | gagtgggaca gctacacctc cagcctccac cttaacttca aacagagtag ttctgtgtcc |
| 59161 | ctctatctta cattctgggc ttcaagStag gactttattt gaacaaagga tatatggttt |
| 59221 | taaaaaaacg ttgaaagcca tcaaccagtg caatcctttc actccgctga gacccacagg |
| 59281 | ggcagtgatc acagaaccag agtgtggtgt agaccagagt ttggatctgc tggttctgag |
| 59341 | ggcagagacc ctacaaactc Ygctagggct ccagggtgcc ctggcagagg tgggttgggt |
| 59401 | ggggcctgcc tctgtctctt agcctgtagt ttcacatcct tagtgcagat cagctgagac |
| 59461 | actggctagg ttcaaggcag tttaggagga ttaaatagca cagagacaat gggagagcca |
| 59521 | caaccttttc gctttctttc accccaatcc ttctgtgtcc ctcagggcta gtaggtcttt |
| 59581 | aataccattt tagtacctgc caatctcctt taaataaagg agaaaagaac tccagttcag |
| 59641 | agtcttcatt tggcaagagt atccggctag agtttaataa cattgttctg tttttattgt |
| 59701 | ttattgcgta cagttacttt ctacttgtga caaataataa atggtttttt ccaattacag |
| 59761 | cagtgatata aagctccctt ttaaattcat ttatttagaa tttatttttt aaatactagg |
| 59821 | taaataatag cacgtgtggg gcatagagat ggctaaatca taaaggtggt agaggaacga |
| 59881 | cttaagctag ggttatggtg ctggaaagga taaacctaga gccagtgggg ggccctggcc |
| 59941 | aatggccaga aagtggatta ctaattacag gttccatgtg attccaagcc gtctgggccc |
| 60001 | tcaggaaagc tccagctccc acagagcacc tcccacctga tttcctcccg gaagcctcac |
| 60061 | ctgctgtttc agcaactcat tctgcgtgac cagctcctgt ttccttagga gggcccctgc |
| 60121 | tccttctggg ctcccaaatg ctgttggtgg agatgatggc ggggtttgga tgctcagtgc |
| 60181 | ttcctccagg gcctggaatc aggaaaagat gggaccatca gcaggaaccc ctggaaatca |
| 60241 | gggccctttg gaggggtcat cctcagggaa ctccttccta ataataatca tactaataat |
| 60301 | agccaccact accagtccta tttaataaat gctgccctat ttatttgcat tctacacata |
| 60361 | tcatttcatt ggatcttctg cacagtgcca tgaagaaaag actatgatca tcatcctcat |
| 60421 | tttacagatg tgaaaactga ggctcagaga cctgaagggg cttgtccaag gtcaaggtca |
| 60481 | cacagctagt aagtggcaga gcacagattc aagcccagca catgctccta cccagcaggc |
| 60541 | taaactgggg ttgtcatgca gaaggggaaa cctccccttc cctgaacatg tgtggctcaa |
| 60601 | ttcttagcac ccagacctag aaggaagatt aagtcctccg ggggaagctt cagtctcccc |
| 60661 | aacaactgcc ctgccctgag attcttcact ttcagcaacc cattcccatc caaaccaccc |
| 60721 | cttccatcct caacacttat gtacatagag aagactggat ttaatttctg cttagttaat |
| 60781 | attttccctt tctaacactg aaaatgggct gaaatatcat caaataacat tttgaaaata |
| 60841 | aaaaaatact cttaatctat catgttcaca atagattata taaacagtgt gcccacatct |
| 60901 | tcttaaaggt ttcagttaac cgtgtttccc ctttaactct gttaacatgt tactcagaac |
| 60961 | tctggaacat gtctcaagac tgggcaccct ggactttggc cccaggacag catcgcccat |
| 61021 | gggcctccct ggaccgaggg gagatagaga cctggcccaa cttggactcg ggcttccacc |
| 61081 | tggtctcctc agctccccta gacacgcaaa gaaactgcag gaccgagagt aaattcagga |
| 61141 | tgctccgagg ggaagttatg gctccacttt atcttcctac tccaccccac ttttaaatga |
| 61201 | aaagactgag gtccagcRac agggagatgc ctaggtcatg agcaaagaag gccacatgcc |
| 61261 | ctgcattact catccacatg gtgtctgtcc ctggctctgc caagaaaacc tccaaggtca |
| 61321 | tctttatgat cagcaacatg gacaattctc tgcctggatt tagagcccca agtcccccag |
| 61381 | ctcaggacag atcgcgtttc tttctctgga aacaggtcaa gggccccaga cttggcataa |
| 61441 | tcagtgttta caaaacacca aaaccaaaaa gaaagggttt ccccagcagg cctaggaatg |
| 61501 | ctcaactcat gccagagtcc actgtcacca gggtcaggag gcagagggaa aggaaaaaca |
| 61561 | attagcacga aacgaaaagc tgagccatga acttaactcc tccaggccct cagagaatgt |
| 61621 | ttcctttcaa agctccctcc tgtcagcttg catctttgaa atgtgctgga aagcaggcag |
| 61681 | aactagagcc gttcccaccN gggctcggat ggcaaaactg aactggagag gctggagagg |
| 61741 | ctaagctggg actagaccac tccgcttggc teagaccaga gggtttctgg tttcctcagc |
| 61801 | ctctaccctg ggggccccat ctccctggat cccactgcac aagggcgggc ccctcagccc |
| 61861 | gtgctcctcc actcccatca gtgtttgctg aacatgtgct tgatgccagg cgctgtacaa |
| 61921 | ggcgcccagg gtatacacct tggtgaaagt atgcacctcc caccaccaga tgggaagaca |
| 61981 | gaattaaata agtgatgacg agccatgaga aggagtgtca gagaagttgc tggtacctca |
| 62041 | ggagcataga ccagggcaac ctgaatgaga ttctgggaag gctttgctga gcaagtgagg |
| 62101 | gtaaaacaaa ccctgacaag cagggtgggc aggtgaggca caaggggaag agtgcacctc |
| 62161 | cagagggaag ggtgacacag gctaattcaa gggctgagtc actcccagtg tgggaggcaa |
| 62221 | tggcagagca ggcgggaaac aggatgccag ctcactgact gggggttacc cttcctaagc |
| 62281 | tgaatggttR ggggtgccag tgatggggtg ggcattgcag ctggactcaa gggagcccag |
| 62341 | ccaagggacc cgggccaagg cacccacctt gttgagccgc tggatctcct tttcctggta |
| 62401 | ctcacgctgt cgggtgagtg ggctcagctg atcctgcagg tacttgacct tttggcgcag |
| 62461 | ctccttggcc tctgctgtca gctgctcctt gtcggtctgc agggaataca aggttggtaa |
| 62521 | gctggctagg atggcctctc caggaggcct cggattctct gtcctgcctg gcccagccct |
| 62581 | tacgttctcc cctggYcatc acttcacaat gctcacatgc aaagcacctg gggtcggtac |
| 62641 | agtaatgtcc atccggtcct gccctcaatt attatgtgtc aatttaaaat aataattaaa |
| 62701 | aaaagaaaaa gcagtagctc agttgagtaa aagaccctgg attccgacac cagttcatcc |
| 62761 | actaagcagc tgagacccct ggcactgcca cagccctctc ttgtctaagc ttacctacct |
| 62821 | gtcacaggta cggggttcct aacgtgagtc acacagagac ctcaggaggc ctgtgagccc |
| 62881 | caaaactgaa tgcgaaattt tgttgtgtgt aatctgctag aaagaaaagc catagttctg |
| 62941 | accagattca catgggggtc ttccgtgatc cacagaaagc gataaactac ggataatccc |
| 63001 | taacagacac tgcagctcat acagccttgg agcctagagg agagatgaca cgcccagaga |
| 63061 | gaactgtgac ataaggcaga gagtgctcct ccagaaagaa acaaaatact gtggggctaa |
| 63121 | aagaaagatc accacagctg agggaaggct tccaggaggc ggttccatac cctctacgga |
| 63181 | accagcaggg cttcgccaca gccagccaag actgggatgg cctttagaca gcatcaagcc |
| 63241 | ccagccctgt aatacgatgg gaaacaaatg tccagagagg ggaagtgacg tacccaaggc |
| 63301 | cacacagtga gctggggcta gaacccaggt ttcctattcc cttgctctct ctccttccat |
| 63361 | gtcaaaggag gtgttgaaac taggggatgg cagcacagtg cagtggaaag accaggcaca |
| 63421 | tccccaaata aactatatat tcctatgtgt acatatatgc gagaaaggtc tggaaacaga |
| 63481 | tgcaccaaaa tggtaacagt ggttccctct agggagggcc tgggatcatg gcagtgggag |
| 63541 | gtagtccagg gaaactttca ctttgtagca tgtggacatt tcataactgt gtattcacag |
| 63601 | catgtgatta agagaccagt cgcaggggtt agttttcacc cagccctgtg tgacctttag |
| 63661 | tcagtacgga ctgcagaggg caggcttggt cagacctccc tgggaattgt tctggactcc |
| 63721 | ccatcaagag gctcccggat gggcatgggc tttaccgaaa ttcatcctta tctacagggc |
| 63781 | cacgccttgt catgaaacgt cctaccttcc ctgtagctga cagggaagag ccctcacatt |
| 63841 | ccagggattt gtctcaccta ccactgtgag caagggagag gggccaaaga gccaggatgt |
| 63901 | ctggctgcga gtcaccaggt atgactgtgc actcataagg aaagacagaa gtgttctccc |
| 63961 | taccacacct ccaaggtaag ggggtgtcac agacgtaggc tgactgctca aataaggccc |
| 64021 | ctggtgtccc atgctgacgg gaggaactSt gaaaggcaat ccaggttgaa aatgtccatt |
| 64081 | tttaagctag gcaaaagacc atgcccagtt cccatgaagg ggaaaacatg gtgacataaa |
| 64141 | taaaatctcc caagagtctg gcagtcaagt ggcctttcct ctttaagctg ctccaaggtc |
| 64201 | ctgatgggcc ttggctggtc tcattcatac atatgactgg gcagatacta ctacttcagg |
| 64261 | ggacctgttg tttcccccca agaaagaatc tggtctcact tatgcccact ctctgatctc |
| 64321 | tgtaacttct tcttcgtagt actttttttt tttttttttg agacagggtc tcattctgtt |
| 64381 | gcccaagctg gagtgtagtg acacaatctc agctcactgc aacctccgcc tcccgggttc |
| 64441 | aagcgattct cgtgcctcag tctaccgagt agctgggact acagacatgc gccaccactc |
| 64501 | ccagttaata tttgtatttt tattagagac ggggtttcac catgttggcc aggctggtct |
| 64561 | caagctcctg atctcaagtg atccgcccac cttggcctcc caaaatgctg tgtttacagg |
| 64621 | catgagccac catgcctggc catacatttt tcttttagag aaacagggtt tcactctgtt |
| 64681 | gtccagtctg gagtgcagtg gcacaatcac agctcactga agcttccaac tcctgggctc |
| 64741 | aagcaatcct cccacattag cctgagtagc taggtctaca ggtgcacact gccacaccca |
| 64801 | gtgaattttt taatttttgt agagacaagg tgtggctatg tcactgaggt tggtctcaaa |
| 64861 | ctcctggcct caagcaatcc tcccacctcg gcatcccaaa gtgccaggat cataggcaca |
| 64921 | agccacccaa cccggccttc tctgcaacac ttaacacact tttactttat ttgcttctgt |
| 64981 | aattagttgt ctggcacctc tttcccagcg agggcagggt tgagctgttc ctgtggactc |
| 65041 | tatgccaaag cacaatgcct gagacaggct ctgaagacaa atgccgggat gaatggataa |
| 65101 | atgaagctct gtctcatgtt gtagcccctc cccaacaaat caccatcact atgctgaaca |
| 65161 | cttggcagca tcccacccca ccaagtgcca tacagcaggc ctgcttggat cttttttttt |
| 65221 | ttttttttta gcaagagttt cactcttgtt ccccaggctg gagtgcgatg gtgcaatctt |
| 65281 | gactcactgc aacctccgcc tcccaggttc aagcaattct cctgcctcag ctacctgagt |
| 65341 | agctggaatt acaggtgcct gctgccacac ctggctaagt tttgtatttt tagtagagat |
| 65401 | gggatttcat catgttggcc acgctgatct tgaactcctg acctcaggta atccacccgt |
| 65461 | ctcggcctcc ccagttgcta ggattgtgag ccatcgtgcc cggccacctg cttagatctt |
| 65521 | ttatatggtg tgcttccctg tacagttccc cattccctac aattttaatg gaacaaaaag |
| 65581 | ttctgaataa aaacaagaaa atgtgaaaag ctgtagttaa tgtaactttc tcaattttca |
| 65641 | gatgggaaac tgcagtgcag ggaaggaacg tgcctgtgtt tttggtcacc tagctctttc |
| 65701 | gcactagacc cccgggtctc ctgactcctg cccctggctt gtttgctcca cagaactctc |
| 65761 | cgctaatgtg gaggggaact gactctgcag ggaccagcag tcacacttgc ctcctccatt |
| 65821 | tcaatcttgg acttggccag gaggagcttg cggtcaaagt cacgctgctt ctgctcccgc |
| 65881 | tcggcctcca ggtcagtcac ctgcttctgc aaatcagcca gcttctgacg cagctcagtg |
| 65941 | atctgggttc agaggcagag aggagtgtgt gaggcaagga tgtagagcac ttgcaggatg |
| 66001 | tgggcagggc ctggctgccc ctgggccctc caacactgcc cccaacttct cctctgggcc |
| 66061 | atactggggc tcagcgKgKc cactcYccaa ggttcaaagc tgctaaagag gcagMgagat |
| 66121 | gaagaccagg catgccctgt gggcgtggcS aggaacccca cattaccttc tgctcatact |
| 66181 | gctgcttcat ggaccggaaa tgctggtccc actgcttgtt cacttccagc agctgtgggg |
| 66241 | agagactgga gttagcagga gatgagcaga gaaggaacaa ggagagccat ctcctgcccc |
| 66301 | atgtacacca gcgtcccaag gtgtgctcgg tgtgtgtgaa tagtggctac agacactgtc |
| 66361 | tccatccctc agctctcgaa aactcaatgt tgctcaaagc catccacctt catccactgc |
| 66421 | tgcattttac cttgccagga aagcaagatt aacactaaga catggaaact acatMgtttt |
| 66481 | ggtgaataat tatggaacac acacacctct ttggagggca gtctgggatt atgtattgaa |
| 66541 | atttcacatt ttcatcaact ttaaaccaat aattccactc tgaagaattt aaccaataga |
| 66601 | aacacttaca caagtgtgca aatatatata cacaaggcta atcatctcag caatacctgt |
| 66661 | attacaaaaa aatccagagg ccaatctaag cacagaagat aggttaaaga aacgatagta |
| 66721 | taaacagaca atgtgcagct gttaagaaaa taagaaaata ggccgccagg cacagtggct |
| 66781 | catgcctata attccagcac tctgggaggc caaggtgggc caattgcttg agttcaggag |
| 66841 | tttgagacca gcctggagaa tatggcaaaa ccccatctct acaaaaaata caaaatttag |
| 66901 | ccgggcatgg tggcacgcat ctatagtctc aactactcag gaggctgaga tgggaggatc |
| 66961 | acctgaactt aggaggtgga ggctgcagta agccgagatc atggcactgc actctagcct |
| 67021 | gggcaaccaa agtgagaccc tatcttaaaa aaaaaaaaga aaagaaaaaa agaaaacaga |
| 67081 | aaataaagca cctaacacac ttcctcagtt tgaagatgac actatccacc atactcttga |
| 67141 | tataacaatt cctcagggaa gaaaaacaac cctacatcat caactaaata aaacaaaagg |
| 67201 | agtattggcc ctgcccctta agatatgtca taaggatcca caccaatttc aaaaacattg |
| 67261 | catatgataa aaaaaattac aaagaattaa aggaatatag catatgttat agatatgaat |
| 67321 | agatactctc aaaggcagtt aatttttttt ttggaggcag gcctaggctg gagtgcagtg |
| 67381 | gtatgatcat ggctcactgc agcctcctga gctcaagtga tcctcccacc tcagctcccc |
| 67441 | caagtagccg agactacagg tgtgtactac cacacccagc taatttattt tttgtagaga |
| 67501 | cagggtcttg ctatgttgcc caggctggtc tcaaactcct ggttgcaagt aatcttcctg |
| 67561 | catcagcctc ccaaagtgct gggattacag gtgtgagcca ccacgcctga cccagttaat |
| 67621 | tttttaaaaa agaaaagaaa aaataatctg taaagcagtg tgtgtatata catatatata |
| 67681 | cacacacgta tatgtataat tttattctac tttgaaaaaa actgagggat attcaacaaa |
| 67741 | ctgtaatcag ttgtggtttt ctctaggagg tggtattctt gatctttttg taatgagcat |
| 67801 | tatatcactt ttgaaatcag aaaaaaagac gcttataaca atacaaaaag aaatctaaaa |
| 67861 | ctaatatgca catcaagaag ctttcaaatg tgcgggagga caaggggagg caggctcagg |
| 67921 | gttctagaga agcacctggg ctgtgtggag aggggctaga cggaacagac agaacaagtg |
| 67981 | tctaccaaaa aggagctgca cttcgacctc gggtctggaa agactggact agactggggg |
| 68041 | tgtgaggagg gggaggaaca catgtgagat ccatgggaag aggcggaatg gactgcagca |
| 68101 | cagggactct gctgcgtggc ttctctcttg gcagcagagg gaaactaaga gcctcttacg |
| 68161 | aactggctcc tgcaccttag tgggactggg ggacatttaa actacctaaa gacctggcca |
| 68221 | tcgccttaat ggagtttagg aatccactca gtattctgtg gcaggcttcg gctgctggct |
| 68281 | ggtccttttc attcttgcct aagaaaattc ttgaaatggg agtctcaaaa tatctgcaaa |
| 68341 | gtagctacac caatagaggt aggtagctat tttttttttt gagatggggt cttgctctgt |
| 68401 | tgcccaggct ggcacgcggt ggcacggtct cggatcactg caacctcagc ctcccgggtt |
| 68461 | caagtaattc tccctgcctc agtctgagtg ggtgggatta caggcatgtg ccaccatgcc |
| 68521 | cgctaatttt ttgtattttt agtaggcaca gggtttcgcc atgttggcca ggttggtccc |
| 68581 | gaacctctga cctcaggtga tccacccgcc tcagcctccc aaagtgctgg gattacaggc |
| 68641 | atgagccacc gcacccagcc aaggtagctt ttaacgttaa aaattttgac agtaaattta |
| 68701 | tggagcctcc aggctagtgg gaagcggcct taatcccttc cttccatgat gggtcctccc |
| 68761 | taaagcatct ctcaagggtc cctccactcc tcatcggtga tgctaaaact tgcctaactt |
| 68821 | atcacaacct ctttccacca tccaaggtct accataggag cctgcctaaa ctgtagcccc |
| 68881 | aacaatttta ggccatgccc cacatgccac ctcctcagag tgaccactgt aggcaaaggg |
| 68941 | actgatcaag agaggacagt ccagggcaag gagtcagacc tcatccatgg gcaggcaggt |
| 69001 | cagtggtcac catgccaact cctcatggtg acctgggacc tgccagagcc aggaggocag |
| 69061 | ggcaagcctt tgtgctgctg gtcccgggag gtaagaggga tcctcccact actgccacct |
| 69121 | cggccaccta cctcactgcg ctgctgctcc agcatcttca ccttcttctc ggctgcRccc |
| 69181 | aaggccacca cctctgggac cttacctgcY gtcacactag cctggggaga agcacagagg |
| 69241 | agccttcacc aaaacctcaa tacaaattat ctcacccagt ctcggtgctc ctctaagacc |
| 69301 | cctcacccaa gKggccccag ccctctgtct ccctattcct cccacccagc aggacaccac |
| 69361 | taaccatccc agtcatgact acgtcccctt ctagaagtct acaagatgat gattccccct |
| 69421 | ccctgactca gggtggctgc caggccctct ccccttccct tctcccctcc ctacggtcat |
| 69481 | tctccctgtt taggtacaac ctctaaggta ctaccagcca ctctcctcac tccctacaca |
| 69541 | catcagccaa aaaactctct ggcctggaag gtaagacatt tgagatgtag atacagacag |
| 69601 | atatagatat agatatactc attctataaa acatatatgt tttataaaac ttttacaaaa |
| 69661 | gttgaagtta tataaaatgt ttatttaaaa acatttttag aaagatgcag aaataaagat |
| 69721 | ctgtgtaata cacatagatg tttcccagct ctgtcctctg taaggggcta aagcagagac |
| 69781 | atccagcagc actgagcaaa cctagtgctc agttggtttc taaataccat tttcttttta |
| 69841 | ttaaaaagaa acagggctgc tttgaggggc tggagcagat aaaatataag gtgagtttga |
| 69901 | tgcatcttat agtgatggaa agtaaggaag tgctcaaatg aataatacta ataacaataa |
| 69961 | taataaaaga gcatgtcaaa agggcacaag agacaacctg aaaaaaacat ctatggccaa |
| 70021 | aacgggaaca ctttgaacag caaaatacat cacaatagta ttggattata agtcacagta |
| 70081 | tgaaataaat actcttgagt tcatactgat agaacaaaat agctgaatca ataaataaat |
| 70141 | gggggagaag ggacagctct tccttacaaa agaattctga ttaataagtg taggaagaat |
| 70201 | gaggaaaata tgaaatcacc attccattac tacagtaata actRctgcag gtggagatcc |
| 70261 | accaatggtc actaaaatca gtgagtgaaa gtttaagaaa aaatagagta tttgcatagt |
| 70321 | ctctaagttt ctcctcaaaa ataaacactt attaattaca aagggaaaat tggtcacttg |
| 70381 | attgcagaaa cttgaaagac agcacttggc caagtgatga aggttaacag tagtaaaaca |
| 70441 | ctgacatcat gtacccctgg tctgctggac tgagaaatat tctgcacact ctgatgtaca |
| 70501 | cagaacgtca cctccgtgag attcttgcca aaacacgtaa cctcaatctc ctcatgagat |
| 70561 | ccaatcactt gtgaggcctc aatcgtgaca gcaaataagt ctatactgaa aaacattcta |
| 70621 | caaataactg gccagtacaa ctcaaaagtt tcaaggtcat gaaaagcaag gaacagctag |
| 70681 | gaactgtcac agactagagg aaaatgaggg agaagagaca actgaatgca atgtgggatc |
| 70741 | ccagactcca tgctggcaca cgacaggaca tgagtgcaaa tctggtgaaa tctgaataaa |
| 70801 | gtctgtagtt tactaagtag tacatgccat taattcctag ttttgatcat ttaacctggg |
| 70861 | ttctataaga tgttgccgtt aSgggaagta aaggaagggg ataggggaac tctctagact |
| 70921 | atttttgcaa ctcttctgta agtctagtta taccaaaata aaaaggttta aaggttaaaa |
| 70981 | aaaaaaatag caagtgctgg ggtgaaacag cagacaaggc cagtagtccg gagggtgtgt |
| 71041 | gtcgactcta tctccaagcc cctctgtgat ggacaccaag accctcgtct tctctgggct |
| 71101 | tcaatgcgat gagaagctca aacagaagaa ggtctgcagg ccccatccgg acttctgaca |
| 71161 | ttgtctgcca tcatcctgga gtcagattct ctctattctc agcgttctct ttccagcgtt |
| 71221 | ctctttccct ccacactgca cccccaagtg gcttcaggta gggctgggtg cagagcttgc |
| 71281 | cccctacagg ctcaggcctg taagcacacc atagcaccaa ggagcaaggt gagggggagc |
| 71341 | cctccaagga aggtcacccc ctttgagaaa ggggacagac aggcctgccc acttgaaatc |
| 71401 | tgcggcatct tggatgggag ctgtgcttag caaaaaggtc aacaagttac cggggaagga |
| 71461 | ggaagagggt ggattccagg ctctagggtg gtgcaccctg aacctccgcg agggagagag |
| 71521 | gagtccacgt ggagtcagca gagctggctg ctggccccaa atctccccta aagagcaaca |
| 71581 | gctaacgttc actgtttacg acgctaaacc tactttacaa gcattgcatc actgaatcct |
| 71641 | cacaaccacc ccttgaggca ggtgttacta tcttcatttt acagaggaaa ctgaggccta |
| 71701 | ggagtcttat gtgacttacc caaggttgca gtgctggtaa aaggtggagc tagtattcca |
| 71761 | cctgcatgca attccaaggc acactcgtcg cccctctgta acacatgcct ctaatgagac |
| 71821 | aggtgaccct ggggcacaag ggcaagtcgc tcccactctc aggaagggca ggaagagcat |
| 71881 | ctggctcggg gcattcaagg aagaYgagtg gagattaaag ggcacaggaa aagtaaagag |
| 71941 | acctgtccag ggaattccac acctgaggca gctcagagaa cccaagggag aacagccgag |
| 72001 | tctacaagtc ccttccacaa gagggcagac aaggccggat ctgatttggg gaataaaggg |
| 72061 | aagaaaagag gatctccatg ggctgggatt cggaacacca gggaagagca gactttcacc |
| 72121 | atttatcagc atgtttccct ggccgtaccc tacatctgcc tgatagcagg tcacacagaa |
| 72181 | atcctgatta tatgatgtta cttgacctct ccaaacctat ggccatagct atagaatgga |
| 72241 | atactagtcc cagccttgcc tactctatag gtagagatga ggcccaaata agaWaataga |
| 72301 | tttgcatgtc ctgggtaaac tctacattgt cacacagtaa taaggaatta agccattacg |
| 72361 | gtaattgcta ctattgcata ggtgtagggt gggttaaaga cattgagccc tactgttcag |
| 72421 | ctattaaacc ccattaaagg gtgaagtaaa aacacgttgg taaagagttg ggactcagct |
| 72481 | ctgcagcaag gggaatggta acgactgggc aaggggtgca gagtctgctc ctgcttggga |
| 72541 | cctggatgtg gccagtgctg ggactagagg ataggggagt Rggccagccc tgcagcatca |
| 72601 | ccagaccaca cagacactag ccaacaccct cccctctcca ctttagaggc tccagagaca |
| 72661 | caatcccagg agcttagcag aatccaaggg agcttgctca accaacctaa ttctgccagg |
| 72721 | aatatcaaag atgttaaact gacttttggg aaccactctc taagacagac cttgacaaac |
| 72781 | tggaatgtNt ttacggaatg cacccagact gagcaaacga ctcaacacca ccttctatga |
| 72841 | gcaatggctg aaggaactgg agctatttag ccaggagaag agaaggctta tgaggcccat |
| 72901 | gaagccacag tgactaccag cagcctctaa gcatggaggt caggccaagY aaagaaaaaa |
| 72961 | acacgtaaca gctagaacct tctaccactg gcactggctg caggagggag gaggctcact |
| 73021 | gtcactggag gtggtaagca gaggctgagt acctgcaacc aaggatgcta cagaaggaat |
| 73081 | ttctgaccac atacctgctg ctgtcYagcc actgccttct tgcctgtccc ttccatcttc |
| 73141 | ggtgagcctg gccgcccaga cRcaccctct ttgttgccat tgctcatcaa caacttcttg |
| 73201 | agctccaaat tttcctccct gggatggagg taaacagaga aatcacaatg ctgaccctga |
| 73261 | gttaagaaac ctMcagggct ccttaatgct cactatcgca tgagttgcag ctgagcccca |
| 73321 | gggagtactg caggatcctg aaacctgccc tcctggaaaa ccaaaaaggg catcttcagc |
| 73381 | taccttgctc tcaatctgtg gaggtctctg attgtcaatg gcagggtttc cccagctgtg |
| 73441 | gaatttctat gcacacaaga ccttccggat atatccaaac actgcattaa ataaccctgg |
| 73501 | atcacattct agtgagaaag tgacaccctt tccaattatc ttcacatttc ttgtgtagac |
| 73561 | cacggagacg gtgcgagctt ggtgctaaca ggccctgggc acctctctag ttactcgcca |
| 73621 | gtttccctcg ttaacacaga aggtgcaggc tgcaggccca gagccttcaa tgtgcaatga |
| 73681 | tccttagcct gaatttatta acatcctttc actttcattg tatttagagt tatcttctat |
| 73741 | ttatcacagg taatgctgtt ttccatttac tctagttaaa gttgccttta taattttaag |
| 73801 | taaaaagatg aatcaaatta aagtaaaatt ctaagtaact aataaggcag gtgatagagg |
| 73861 | agatcataaa attagtgatg tggattcagg tctgactaaa atgtttcttc tttctttttt |
| 73921 | ttgagatagg gtctcgttct gtcacccagg ctggagtaca gtggcccaat ctcggctcac |
| 73981 | cacagcctcg actgctccta ctcaagtaat cctcccacct cagccacccc caacctcccc |
| 74041 | caccacacac ccccaccctc ttgagtagct gggactacag gcacaccacc acacccagct |
| 74101 | aatttttttg gtattttttg ttggggctgg Rggaggggtg gtgatgtatt tcaccaggct |
| 74161 | ggtctcaaac tcatggactc aaagcaatcc tcccgcctta acgtcccaaa gtgctgggat |
| 74221 | tacaggtgtg agccacaacg cccaaccacc acttctttat gttctatttg ccagctgagt |
| 74281 | ttccgcttga aaaaccaata agtaaaatga ctttcccaag gtcatacagc agagacttgc |
| 74341 | attcttgcct tctaatccgt gacttttttc cccagcttca gtttctgaat ccatggatgg |
| 74401 | ggtcaYtagg gctataaaaa ccccaaaagt gcatccttgg ccatccaaaa gcaaataaat |
| 74461 | tacacactga ttcaaaatag ccagtggctc agtagttaaa cattacttct gtggccatac |
| 74521 | ttacaaagtt tatattcctt ctctttctgt tgattctaat aagaaacagg agaggaggga |
| 74581 | aaaacagagt ggcaagtcct gtctgtgaca cgggcctttc ctatgtgcct gagcccagct |
| 74641 | ggtacccacc agatgactca tcacYcaaac tgtccaggac cacagtgcag gcaggaattg |
| 74701 | tggccactca caggatggag agcaaggagg cccaagttcY tgttgtaaga ctccaatcaa |
| 74761 | gtccctgcct ctccctgggc ctcagtttcc cctctgcaca gtgagaggtc tgtctgcatg |
| 74821 | atctccaagg gcttgtcaag ctctgacagt caagatcagt gtttcccaag gcgtgatcct |
| 74881 | gtgcctacct cgttcataat cacagagact cctggtgaaa tgcaggctcc taggtcttgc |
| 74941 | ctaagacctg tcaattcaga atcttgcaaa ggaaggaagg gaaggcgtat aaacttgtaa |
| 75001 | tgcctttccc tgaccctcaa ccctcagtta agtctaagcc ccctagtcca gtgggggagc |
| 75061 | tctcatgttc tcaggaagac agagcaggtt gggatggtga ccagcaggag gcagcaaagt |
| 75121 | cctgagtcct ctggacagcc agttcaccgt taacagggga gagaggcaag tgctggagga |
| 75181 | ggaaggaagt acaatctatg ctgaccttct tctgaagctc tcaaggcact ctatacacct |
| 75241 | cccaaggtgg caaaagggcc agagagaaaa taccaaaaga gccaaggaga aggatcaaga |
| 75301 | gacgtaacct ccaaataggg ctctgggcac agggcctcag agccacggtc ctcaacccag |
| 75361 | aaatcagtgc tgcacaccag cYcctcctcc tgcagccccM actcctcacc ccttctctga |
| 75421 | ggggcctgaa cttaccgcag cctctcggca gcctgatccY gctgttccag gcccttatcc |
| 75481 | aacttggcct tcagagcctc gttctccttc cgaagctgct cacacagggt ctgtggggca |
| 75541 | ggggaacaga cagggtgagg gaaaggaggg gcccctccca ggtgcaggct gagctagagg |
| 75601 | atggtggggg gcctgtagcc accccagggc ccaactcctt atcccctttg cctggcctca |
| 75661 | ccccatccct ctctaaggag ctaagccact tggccaccca cgtgtcctct cccttgggag |
| 75721 | ccaactacgg agaagcctga ccaagtatca gccatggggc cttctgctcc tccaagacag |
| 75781 | acagctccat cccttctcct tcatccctgc tcccactgcc gggaccccta ttccccacca |
| 75841 | gcctccccca gctgacttct acYcttcttt gaagtcttag tttaaatccc atctcctcag |
| 75901 | agaagtcctc cctgactccc cgatccagct acattaggct aactccgtta tccttgctca |
| 75961 | cagccccccg cttttacgct tctcagcacc tgtggcagat aaagctaaag aactgttcgt |
| 76021 | gctgctgttt tcttaatatc taccccctta cccctaatta attcctgagc tcccggaggg |
| 76081 | ctgggacccc taaccatYct cttgttcact gccagatccc cagcccccag ctcaaagttt |
| 76141 | gtggaaggaa caacatgact gaacagccaa atgagcaaat ggacaaatga gcaggcgagt |
| 76201 | cctagggggg ctgaaaggca caccaatttg caggtgtgag aggtgcccag tgttgccaag |
| 76261 | gggaaaacca gaagacaagg cattcagtcg ggacctcact atgccctcac gccctcacaa |
| 76321 | tgtggctgtg acacttggtt cctcctctac aaaagaaaga caactgtttc tctttttttt |
| 76381 | tttttttttt ttttttttaa gacagagtct cactctgttg cccaggctgg actgcagtag |
| 76441 | catgatctca gctcactgca acctccacct cccaggttca agagattctc ctgcctcagc |
| 76501 | ctcctgagta gctggaacta caggtgtgtg ccaccatgcc tggctaattt ttgtattttc |
| 76561 | agtacagacg gggtttcacc atgttggcca ggctggtatc aaactcctga cctcaagtga |
| 76621 | tccacctgcc tcagtctccc aaagtgctgg gattacaggc atgagccacc acacctagcc |
| 76661 | cagggacaac tgtttcttct ctgcctcctc tatagggctg ctgagaggga cagatgagac |
| 76741 | agtgagagtg gaagtgtcca tgaaccatca gtcacttgct tatgtgcggg gactcatatt |
| 76001 | ggaagctctg tgaactgggg gcatggaaga ggggtcgcca gactcaggac ctcacaggct |
| 76861 | ggggggaagg gaaggtgctg actagcaatg ctagcataca gtgtcagcac agagttgttc |
| 76921 | aatctttcca cagatcctca ctgagcacct cacctgtgcc aggtacattg ctcggtgttt |
| 76981 | ctccatatac tgcctcactg gtgcctcaca acttcctcaa agctaagtaa ctcatctcta |
| 77041 | tgttaaaggt agggaaacca ggtaaaatga tacagtctaa caggtgcaca gctgctaaga |
| 77101 | ctcatcactg gcggggagta aaactccaat tggcagaccc cacactggtg tctctttccc |
| 77161 | ctgtgatgaa agatcagaga agggagagag atggtgccca aaggagggga cagcctttga |
| 77221 | gctgggccag gaccagggcc attcactaaa cagccatctg ctgccttcca cctcccaggc |
| 77281 | ctgttgcagg tggatgcaac catgtgacta gttctggcca acaaaagagg tgtagaaggg |
| 77341 | acaagcatca ctcctggact ggcctacgca caattctcca gtctcttttt cctggtagca |
| 77401 | ggggctgatg tggatgcctc gtattctaaa gggcaccgga aagttgggcc cctgtctccc |
| 77461 | tgggactcta aggaactgtg tagagcagaa acaaacccct gccacacaga ataggaggga |
| 77521 | aaacttttgt tgtgtaaccc agggagattt cagagttgtt tcatttctga ctcacaaagg |
| 77581 | agactcttgg gaaggctgaa tggaggacac agccttcaac tggaggtggc tggacaatga |
| 77641 | gggagatgag gcttagcaaa cagagaacca gctgggcatg gtggctcaca ccagtaatcc |
| 77701 | cagcactttg ggaggctgag gcaggaggat cacttgagcc caggagttca acaccagtct |
| 77761 | gggcaacatg gcaaaaccct gtctctacta aaaataccaa aaaattggcc aggcataagg |
| 77821 | gcgcatgcat gtagtctcag ctattcaggg aactgaggtg ggagaactga gcccaggagg |
| 77881 | tcaaggctgt agtgagctgt ggtcgcacca ctgcactcca gcctgggtgg cggagcaaga |
| 77941 | ccctgtcccc caacacccct taaaaaaaag aaatcaaaaa attattgtta tgagtccatt |
| 78001 | caatgcctag cagagagcct tatgctatac ctgaccttga atatgtttgt ttagtgaacc |
| 78061 | aagaaacaca tcagtgaatg aatgaggagg aactggatca atacacctcc acccaggcag |
| 78121 | taaaaatacc caagatgcca tgtctctggg cgaggcctcc agggaacaga atggcctgct |
| 78181 | cctagtccct aagggaaggc caggacaagg cctcacttgc tgtcctaccc ttgcagtcta |
| 78241 | gagagctgtt tccttggagt ctctgggcca atgccctggg tgcaaaccgg ccaatggata |
| 78301 | gaatcatcag aggtcacaaa gatcatcacc ttccatccaa ggtgctacga gagctatgag |
| 78361 | ttcatttgat catttattat tttgtccttt cattcattca ccaagtgtgt atgaagtgcc |
| 78421 | acgaactggg aYgcaacagt gaatacaaca gacatggtgc ctgcccccct caagcttacc |
| 78481 | ttctagcaga gagtataaga aaaaaaagag tataagaaaa aatgtattaa ataaaattat |
| 78541 | tacaaactat gataagagct aagaaggaaa taaacaagga ggggactgaa ataaaaaata |
| 78601 | acaKaggttg gcaggacccc ttttaagggg cctaggatct atccccatct caaagctgaa |
| 78661 | aagtggatgc gcaaagtgct ctcaccccag gcaccttcct accctgccca cagcctaggc |
| 78721 | actgtgccag gctcaccttg ccagggttca gcctggatac cttgaagcct cactgacatc |
| 78781 | ttaaaggggt tcccttctct cagcctggct ctccaggcct ctgcctctcg ttcagcctca |
| 78841 | cctcccaaca cacccctccc ctccaggtgg gatgggcatc ctccgtgctc ctctaccccc |
| 78901 | acacctctcc tcatactgca tctccaacct gattgccctt catgccctcg gcaaccaagg |
| 78961 | tcacgcaagg gggactccca cttctgccat cagcggggca cttgagtgcc tgctgacatg |
| 79021 | aatgagacca cagatcagag aatggcctgg gaggagagtc tgcagggagt aaggaggctt |
| 79081 | caagtcatgg ctctgtcact aaggtggcgg gcactcagat cacgagcagt ccctcagaag |
| 79141 | cctccgtttt gccatcttta gaatggtcac caattataac tgaacacttg cccgctgcta |
| 79201 | aagcccacaa ggctaagacc ccctgcccag tgtggatgcc cgtcatgaac tccgaatggg |
| 79261 | gacctctctt ccagcatgga ctaggttctg gactgcactg ggtgcttcat gtacatggcc |
| 79321 | tcatttaatg cagcaatcta accatggagg cacttctagc tcatgttact gataggaaca |
| 79381 | ttgggggata gagaggataa gtgacttgtc caaggtcata cagcaataaa gggtcactac |
| 79441 | tggaaccaag gaccatataa ctccaaggtc tggcctggcc tagcaaggaa atctgtccat |
| 79501 | tccccacagc acagggaggc tttcaaaagg aattcaatag gggaggcagg ttgggatggt |
| 79561 | ggtcaagagt atgggctttg gaatgcgaga gatcgaggtt ccaatcccta cttcaccatt |
| 79621 | taccagctgg gtgaccttgg gcaaaaaaag ggctgcttcc ccacctgtga agtggtgata |
| 79681 | atgagggtgc ctaccctggg gggaaagagg cattatgaaa ggagttgacc tttgttccct |
| 79741 | cccttcccat cccccaaccc tgcacatgag aaataaaggc agggctgctg ggggtatccc |
| 79801 | caaccatgac actcgcattg cccatcccgt atttccccat ttccttcctc ttcctcatcc |
| 79861 | ctccccacaN cttccttatc ctgttcatca ccctgaacaa aaagcccgaa gtctgcccac |
| 79921 | aacacagSct tcagaagtcc ccatggtacc agtggattcc caggagcagg aaggtgggaa |
| 79981 | gagctcgggg ggaagcacgc ctgcctgtct cgggctgccc tccccacgcg cctcacctgc |
| 80041 | agaatggagg tgcgctgctc attcttgtgc accttggatg ccagtcggtt gaactccagg |
| 80101 | gccatgcggc ccaggtgggt gaagagctgg ccgtggtccg gctcctcggc acacacactc |
| 80161 | agcgtggtct ccaggcgctg caggtgcagc atcaggttgc cgtcctcacg ggScaggggg |
| 80221 | cccagcRcct ggagagggaa agggcacacg gcccactctt caccaaggcc tgagtaggcg |
| 80281 | ccgccagtcc attctccctc tgcttcctca tttctcatga ctcagtttcc ccctgtgacc |
| 80341 | ccagctcagc cccgggaaat tctccagtta tgcaggttcc ctgtctgggg ctgactcaca |
| 80401 | gggcagaacc caggactcac aggccactgg tggctccaca gcagcggggc caccatcctc |
| 80461 | ccacacactg ggccttgcag agtgcctgac agtaagtcac ttgtctgctg gacctcagca |
| 80521 | tcctcatcta ccaaatgtcc gcacaaccct tccctccctt caacctcaca gggttcttag |
| 80581 | gaggatgaat tgacccctga cagtaacaag cccagaaaag ggacatcact gcacagtcag |
| 80641 | ccctccatat ccatgggttc cgcatccatg gattcaacca actgcagatc aaaaatcttc |
| 80701 | taaaaaaaca aactggccgg gcgcgatggc tcacgcctgt aatcccacca ctttgggagg |
| 80761 | ctgagcgggc agatcacaag gtcaggagat caagacggcc tgagtggtga aaccctgtct |
| 80821 | ctactaaaaa tacaaaaatt agctgggcat ggtggcacgt gcctgtaatc ccagctactt |
| 80881 | gggaggctga ggcaggagaa ctatttgaac cagggagtcg gaggttgcag tgagcagaga |
| 80941 | tcgcaccaca gcactccagc ctgggcaaca gagcaagatt ccgtcccaaa acaaacaaac |
| 81001 | aaaaaactgt attgaacatg tacagaattt ttttcttgtc attattccct aaacaataca |
| 81061 | gtataccaaa atttagcatt tacattgtat taggtatcgt aagtaatcta gagatgattt |
| 81121 | aaagtatata ggaggatgtg tataggttat agggaactac tacgccattt tacatcaggg |
| 81181 | acttgagcat ccatggattt tgggtatcct cagggagttc tggaaccact ctcccatgtt |
| 81241 | taccaaggga catctgcata tacgtcctgc aacctccgcc tcctgggttc aagcgattct |
| 81301 | cctgcctcag gctcctgagg agctgggatt acaggtgtgc accactacac caagctaatt |
| 81361 | tttgtatttt tagttgagac ggggtttcac cacaatggcc aggctggtct tgaactcctg |
| 81421 | acctcaggtg atccacccac ctcggcctcc caaagtactg agattacagg cgtaagccac |
| 81481 | catgcctggc ctacacatgt gtcttctggg gaatggccca agcattcatc agcttctgag |
| 81541 | gacccaagac tcaaaacagt ttaaggaacc acagctttag ctatttcaga aagtcccaaa |
| 81601 | ctagtacagt atgtaagaaa taaagcacac cgtgggccag atagttgact ctttctctct |
| 81661 | agtctatctg gaatactcct accaaagaca ttgcctttgt tacaccaatt ccactcccaa |
| 81721 | ggacctgcag tgttacccta ctgcagacgg cactgggccc agctcccctg ctgggcatct |
| 81781 | aaggccctac agccgcaggt cccatccaca gcagcatcca gttgggctcc ccactttacc |
| 81841 | ctgcctgaac ccacacacac caggtctggt gtctctgcat gtgtcctctc tgtgtcctct |
| 81901 | ctgcctctgc aagttctgct catccttccc accctgtcca cggctcagct cctccagcag |
| 81961 | cctcccggaa acagttatgg gaatcacagg acaKcagagc tacagggagc ctcagagatc |
| 82021 | aggccgggaa accaagctat ggggaggagg aacttgccca aagttgccct gtaagtcagt |
| 82081 | ggcagaggta taacgagaac tgaggtttcc tgactcgcca tccagtgctc tgctgcactg |
| 82141 | acctcccact tctctgaact ctggaggaat ggcaatgact ataaccMcaa cttggcattc |
| 82201 | cctgaacacc tgtgtaatgg cgtcagtgtg ccgcattcta acaagaggaa atgctgctgt |
| 82261 | ttcaactctg catccattcc cccactgcct ctttccacaa aggagctgag gtggcagatg |
| 82321 | ctgctctatt taaggctcat gcgatgcttc ccatgcctgc gacttctcca gactacaagc |
| 82381 | ttcagagcac agggaggctg tgttgtcttg gctcaagtgg ctcagcagag acaggcacac |
| 82441 | tgtagagcta atgaggaact tactacgtca gttaaagtta tcggtccaaa gtcagagtga |
| 82501 | aacactttct gagcagggga tcttgcctcc atggccagca tctcagagcc ctcagggaca |
| 82561 | gaacaggaca aggggacaga aggttccagg cgacagaaca taaagagcct ttccaatact |
| 82621 | cagagctgcc tgaggcagtg gtgagcctct caacactgaa ggcatgctag ttagccagac |
| 82681 | agccacctga gggggccYtg cactgggcac tgacaggtac atggggcaac cagtgaacac |
| 82741 | aagcatattt gtcagccaaa cctttgtgaa gggaggcagc agggatcaat atatcagtga |
| 82801 | agggctagag cacctattgc tcgcataaca acaattaaag Raaaaaactt aaaagtggga |
| 82861 | aggaagggag aatggagaag gtagaaattt aagtcgttta aagaatcttc cagacttttc |
| 82921 | acaatggttc ttcaataagt ctgccatttt aagaaagaac agaaggagat atagttagga |
| 82981 | aaaaggcagc cagtgttgta aggtgtggaa tgaggatgct gggcagacgc tccagagctg |
| 83041 | tacgagagac agagagagag agagagagag agagagagag agagagagag agagagagag |
| 83101 | tgtgtgtgtg tgtgtgtgtg tgtgtgtgtg tgtgtgtgcg cgcgcgcgcg cgcatgcgtg |
| 83161 | taagtggaaa gttgaacctc cttcctgtgt gctccaagag tctgcacaaa gagYaaatca |
| 83221 | cctgtcttac agggccctgg gccttcctaa cccttctttc tcccccgggg ttggaagccc |
| 83281 | acagacccaa agggctgagt catagtgtgc ggaggtgcct ggcccctggc agaaggcaac |
| 83341 | agggctggca aagagcccag gctttcacaa cggtcgggtg cccgtgtgct ccaagtcggg |
| 83401 | gaagtcacta gcccaggaat ttgcatatgt gagcgagaag caccctttgc tcctgcccct |
| 83461 | cgtgtatccc caagtgactc caggRtacct aasggatctg aacaggttct gtgcctctca |
| 83521 | catggtagca agtgacagga cacaaagagg gcagcaggtc aagcccgggg tcctgcccgt |
| 83581 | ccactcacca tcgcattggc atggctgctg ctctctggtg aattctgctc ctcaggagtg |
| 83641 | accacttcaa attcagagga ggtgccctgt gcagaaagga agttaggtgc tgcccgagag |
| 83701 | aaaaacagct cacccctcct ctgcggccct gtggctgaga gcagcaaggg ggacaaaatc |
| 83761 | tcttcccatt ttgagtacac agggaccgtt acatatcatc tcacatRaga tcacaaagca |
| 83821 | ggattctcga caagcctgtc atagccctct gcataatgtg aggtcatatc ccctcgatgg |
| 83881 | ccaatcctct tttgtagaag ggagatRtgg gactagctga ccttgaacca gcagcactgt |
| 83941 | aggattgcat tttttaaggc cgtgtacttt ttaaatgctt tcatgagctc cattatttct |
| 84001 | cagggtgacc catggagtgg gtcagaaggt tatatcctct ttggaaagat gaggaaacta |
| 84061 | cagtatacag ttggttaatg caaaagcagg gattgaaccc aagactgcag atccagcatt |
| 84121 | cctgccacgg ccccagagct gtctcttagg cccagacgtc tctgatcggt ttcccagaac |
| 84181 | acagatggca gctgttacgg ccaccaccaa gcatgYcctg tgctggcctt cccaagccag |
| 84241 | tcctggagat cccagaggag tgaactgatc tgagaaaagt cccagagatg attcaccaaa |
| 84301 | tttggagact atttcaggat tcaaccaaat ccacggtggc caagaaatcc cagatttttt |
| 84361 | cacatctttc cgtcattcct agtagaatta ctggtcattc aaccaaccaa gaagccaatg |
| 84421 | gcccacctga accagccatt agctctggga tctgggattt ccatgtcccg ttaaacctgg |
| 84481 | tctgcagcct cccttcatcc cagaaagggg caggatagat gagaggagtc agtgctcagc |
| 84541 | ttccagctga ccctcatggc caccaagaac tcacctctgg ctcttggccc tgccctagaa |
| 84601 | agtctccctc ctccatctac attttcccca ttacccgtgt aacctcagaa caggtYcttc |
| 84661 | tttccctgca cctgatctgt aggataagga tgctgaacta tgcaatccct tgtaggatac |
| 84721 | caagcaaaac ttctccagcc aaatctgtag attttttttt ttttttatga gagggtctga |
| 84781 | ctctgtcacc caggctggag tgtggtggca tcaacatggc tcactgcagc cttgacctcc |
| 84841 | tgggctcaag cgacccttct gcctcagcca gcctcccaag tagctggaac cacagacata |
| 84901 | caccatcaca cctgactact ttttttctta gagagatgga gtctcactat cctgcccagg |
| 84961 | ctggtctcaa actcctgggt tcaagcaatc ctcctgcctc agcctcccca agtgctggga |
| 85021 | ttataggcat gagccactgc gcccagcctg tagaagatag tttctgagcc ctctttgaga |
| 85081 | atgtgagctc catgtcacat ctgtctgttt tcctagcact gtgcacactc agtgatactt |
| 85141 | aacagtatac aacttagtaa gtgagaagaa ttccttgtac ataccagggt cttcctggta |
| 85201 | agttttaact atgaccttca tttcaaaatg gggaggctga ggccccagag agtttgcgat |
| 85261 | ggacttgccc aatatcaccc agcgagacgg aacacagttt cttgacctca accctctttc |
| 85321 | ttcatgttct tgtatctgtc agtaggactt gaggtccatc caggcaacct ccacccatga |
| 85381 | ctccaaataa aacttacact ggatggaggc ttctggactg gtgctggctt gtcactgggg |
| 85441 | catgcagRgg ctgtgggaga tgctgtgaca tttgagtcct ttcctggaga atcaagaaag |
| 85501 | cacagatgaa ctgactggga aggggagaag cccaggtacc accctctcaa ggacagccct |
| 85561 | tgacaaaagg gttctcagac aggattcccc actcgagtgt gggagaatgg gaggtggtct |
| 85621 | catcagggcc aggtagtctg agaaaatggc ctgccctgga gtgacggggc agagcctgag |
| 85681 | cagagagacc atgctccctg ggcttactgg gaaaatccca agttggctgc tgctccttgc |
| 85741 | tgccctgaac aggcctcaat gataaagaca atgatttaca atttgaaaag tctttatgta |
| 85801 | tctattatcc cagccattct cataaaagcc ccatatcccc attttaaagg tcaggacact |
| 85861 | gaggcttggg aaggtcatca Mcttgcccac agccacatag ccagcaactg caaactcagg |
| 85921 | actgagactt ggccctccct ccctgacttc ctgcagtcct tgctgcaggg agctccttga |
| 85981 | aggtagcttc atttctcact ctgagagtgg acctggcacc aatcaggaac tcaggaacat |
| 86041 | tgctgaatag acaagcagag gctggaaatg cccactgact gcagcacagt ggactctgag |
| 86101 | accatcactt tcctccattt gtccattcag caaatgcaac caccagccaa tagaaggctg |
| 86161 | tcacattggc ctgggccaga taccatacag tagagctgca gcaggaggca tctgagtcca |
| 86221 | gctgaggtgc ggatcatttc catcctggtc ccagacctta ccctggactt ggagacatgc |
| 86281 | cagagtaatg cacactctgt acgcatggtg atgcctctac catacatggg acttcaaccc |
| 86341 | cgagccacag tcaccctgtc ctttaatggg cagcacacgt aaccttacat ctgaaatacg |
| 86401 | ctgcagcagg ctaacaggca agcccagctt cttccctggg cctcagcttc cctaattgta |
| 86461 | aaatggacgc gcagggcacg accagggagc ttccaactca tatgccagct ccaagtgaca |
| 86521 | gggagtagct gcttggagYg cgatgcctgg cacacagtag ctgctcatta aatattaaat |
| 86581 | atgctgataa agaccccaag gccacaggac tgcaatgtag tcaactgttg tatctattac |
| 86641 | tcatgtctgc tggggcctca ggagtgctga gtcactggct atttttaaca agattgtttt |
| 86701 | gaagtccctt ccaaatctga ccttctgggg gatgtggcag ccaaaacatg aatggaaggg |
| 86761 | tagcctgtga cctaaggata aacgagagaa tgtggRtttt ggcataagaa gggagttcac |
| 86821 | tgtgaaggat ttgggcagtt tggggtacag ggagagtcag aggtacccag actcctctta |
| 86881 | tacctgtgag ctcagccagg gggtcgaagg agcccaagga gggagaaggt ggtgggagca |
| 86941 | gctcgttgtc cttcactagc tcctctgcct tctgccggag cctggtcgct tccatctggg |
| 87001 | actcttccaa aagctcccct agagttattg gggaagagga agcaaaagca tttactYggc |
| 87061 | tcagtgtgct gcttctcccc gtcccaggcc cctaaccgtg ctgccgcctg gcttctgagg |
| 87121 | ctccaggccc tggacttcac tcccaccgtt gctctgtggg ccaagtggga caaactccat |
| 87181 | gctggtccac cataagaaac aggctagagc aaggacgagg gcccagcaat cgggggacac |
| 87241 | aggagcggaa ggaatctatg agaaactcct cggctccccc aacccccagc attctctttc |
| 87301 | tgccccactc ccttcacacc cagccagggc actccacaga aagccagggg ctcccccacc |
| 87361 | ctacgggcta cccacagcct gagccaccag ggcaaagggc tgcctgcagg gcggagacct |
| 87421 | tggataattc caaccagccc tctaggcctc cattttcctt ctcctacaag tggagatggg |
| 87481 | gaaaccactc ctccctcccc agtcggccat cccaagcaag gatctgccaa gcaaggagaa |
| 87541 | agcattttaa aaatcagggg gctatgctcc cctctaaaac atgagactag agaagggcac |
| 87601 | ggcaaggcaa aggaagagtt tcaggagtgt ggaagaaaga gagccccctc tggatgggga |
| 87661 | aatcaatgaa agccttgtct ggaggagaag ggggcacagc aactgggtgc tgatgcacag |
| 87721 | ggcccgtcgg tcgtgtggag ggaggtgggg ttgttctaaa gtggggatgg cttgagcaag |
| 87781 | agccaggaga agagaactga gggacctacc acctccttgc ttcaagccaa gctctgctcc |
| 87841 | ctgctccaac acctaggaac gcgtgcccca gttgcctggt catgctggct ctccccatct |
| 87901 | ctcgtgcccc agaaagagga gRccactcta gttccatggc cattaactgg gccttccagc |
| 87961 | cccctttccc ttagcccacg cctccctctt ggacctgagt cctgtcatcc tggtttccgg |
| 88021 | gtgctgagga atcagggttg ggaatactgt caccatgcta catggtgcat agagacagct |
| 88081 | gtgaggcagg agccagcRct gggccaggag ggacagggga tgacttgggc agaggcatat |
| 88141 | gccagcttca ttctccactg ctagtagagg gactggcatc acagtctgca gggaggggcg |
| 88201 | ctcgcaggga ggggacaggg tctgctttac ctaacatctt tatcccttgc attttttcct |
| 88261 | tcagccggga attctccttc actaggcgct caaaagctgc ggatgcctct cctgagggca |
| 88321 | cgctgccccc agggtcgtag atccggtacg gtcctctccc ttccatgagg gtagctcagc |
| 88381 | ccctgccgtg gtgcccgcct ggctgtaagg acaacagcag catatcagca ggctggccag |
| 88441 | gccatggggg catcctttgc agagaagctc tagtctgtca atgccccaga aggcagtccc |
| 88501 | ccactttaag ctggtctgac catattatag taaagcagga cagtcacctc cttttatctg |
| 88561 | gacgctctgc ctctattaat acaaccttag aatggcatta gtttctctta gcagcctcaa |
| 88621 | tgcactgttg gcttgtttgg aacaggagtc caaataaaca gtagggtgca atgtgggata |
| 88681 | caagaaagaa cctggacttg gaaaaaaaaa aatctgagtt catgtcctgt ctgtgaggtc |
| 88741 | cagaagaaca ggaatcccat atgttttacc catcattgca tcctcagagc ctaccacagt |
| 88801 | attagctatg aattttgccc aataaacact tactcaatta atggtctagg cattaattta |
| 88861 | ctatgggatt atgggcagtc ccttcccctc tctgagggag ggaaatgaaa gtcttaagtt |
| 88921 | aatttagtaa tctttaaagt gaaatgcttg taccctaagg gataagcaag tcaacccagt |
| 88981 | ccaaagttcc atttatatgt gtatcatctt ctttaaaaaa agaaaagaaa aaaacaccct |
| 89041 | gactaatatt taatacactg tcaccctcct ccgtgagtct catatcatat atggtctgtc |
| 89101 | ccttgcagtt ctaaggaaca gaagaatttt tcatcaaacc actgtctatt tgagcaccaa |
| 89161 | ggtttttttt tagtaatctg ttcagtttca tctgctttat gtcactgaaa ccagttggaa |
| 89221 | aggtgatttt agaatcctaa ctctgtggct tagtagagaa gctctctgtc cattcactca |
| 89281 | tccattcaac aattacttat tgagcatcga ctccgtgcKa ggcaccagtc tgggtgctga |
| 89341 | ggaattatca gcaaataaaa cagacaaaaa tccttcatgg agcttatagt ctaggggtga |
| 89401 | aagcagagga ggatggtggg gaacacaata tttcaggtaa tcaaaaagtc tatgcacaaa |
| 89461 | aggccccaag cataaggagc tattttagcc aagacctgaa agaggtgagg gaactagcca |
| 89521 | tgaagacatc tgaaggaaga acattctttg cagagagaac agtaagggca gaggccctaa |
| 89581 | ggctgaaaca ccgagccagc ctccagccct ccaaatagct ctccagcatg aaagggccaa |
| 89641 | ggccttcact ggggacctca gaagggtttg ggggctcRgc accttcctcc caaggtcacc |
| 89701 | acccccaaga caaaaactgt ttcatcagga atcctcaaag accctcccaa gtccacacac |
| 89761 | catgctccgt gggtccttgg cgctgccagg tgactacgac cataccaccc tgctcccaga |
| 89821 | atgtaaatga aagatcagag tgagaaagga ttaacagcaa caccaccatg cttgaatcgc |
| 89881 | ctccttgtca ggcactgcat taaatgtttc agatatacaa tcttggagga gctgcacagt |
| 89941 | aactctaaca tgcatactaa tctcatctgc attttacaag ggagaaactg aggctcaggg |
| 90001 | cggttaagta actcagccac gatgaMccgg ccRcaataag agccaagatg caaagtcagc |
| 90061 | cctgcctgct gtgactctga cccccaccat actcacacag ccaggacaca gagScagagg |
| 90121 | ggcagacagg gagttcaaat ctctttgtag ggcagcacca tgctatcagg tcacctctgg |
| 90181 | gcagtttccc ttgccctgca gcataagaac caataaggac ttgtccaaga gcgtccatag |
| 90241 | gatggaatgg gccagaaaga aaaacaaagt tctgatgaga aggacccctt ctcccaaaga |
| 90301 | ctgacagata caagatgaat acaccRtgtg ggaggcaccc acgccctgct gagcctgcga |
| 90361 | ggcaaaggac agctacccac actcccatca caagtcatca cccggcttag gggtgacttg |
| 90421 | tttttaacct gaaaggtcag tgttctacag catagcacaa agYgcagatg tttccaccga |
| 90481 | actaagaaac agtgtggggg ctgcacttag cactggaact ggcacatagt aaagtactca |
| 90541 | aaaatgtYgg cagacattgg gggtccttcc cctctgcttg cctctgtcct cacgaggctt |
| 90601 | gccacctggt acagcatgtg tctgactggt gcgggcattg cccatctggc tgggaacttc |
| 90661 | ctgaggcagg agcgtacttt gcacatcttt gtattcctgt cgggagcaca ggagggtggg |
| 90721 | ccttccttga gagctgcttg gagttcaaga acagtgaaac agagggctac aaggggcctg |
| 90781 | gagccacctt gcatgacaaR ggcagcagtg tcctggttct tgtccaccat tatgcatKca |
| 90841 | ccctaaatgg aactcacagt caacaccaca tgaaggcatg gggaatttct tactcaatgt |
| 90901 | ctgccttgtc ctgtagatta gaggctgcat gaggacaggg agcaagtcca tcctgtgtgc |
| 90961 | cagggtatct tcagagtggc ccagagtagg gcctcggtaa accttgaatt cagagaagtc |
| 91021 | agactgaagg gaacaagccc ttgaccccga ccccgcctcc tgcaaggcca tgtccaacat |
| 91081 | cccagagaaa gcctaaccct gacccactga ccagacgcac ttggacgccc agacacttct |
| 91141 | agaatccagg ctgggccagc catgctccct cagagaggcc cagatgtaag gcgaagggac |
| 91201 | aaagcaggat gcaggatgtg ggaactgaaa cccaccccac gtcctccaaa acacgcaaca |
| 91261 | ctcagtttct cttggcgctg gggagtcaac gggaaagggg ttcatgggaa acaaaaagtg |
| 91321 | ggcagacaga gctatgctcc cctactttag cctcaggggg cagagcttgg ggagaaagaa |
| 91381 | aggcagtggg cagaggagaS gcaggagaaa tatcctattg tgggaaaacc ctctctggag |
| 91441 | atcagaggag ccacccctgg agggcctgga cttctggagg aaagaaagac aaagagggaa |
| 91501 | acagggaatt tccaagttct gctgacctca tgcccagatg gtcagcggca gtccctggat |
| 91561 | gaccactggg cagacgggaa ctctccctct gatcaatgag gtttttataa cttctcccag |
| 91621 | agagaggact cattctcagg atgacagagc tggaggggac tctccaaggg caatgaggcg |
| 91681 | aatcctgcat tatcaggtgg gagagagatg cacccagaaa cactcaatgg atgaggggct |
| 91741 | gcattcgaac atgacgatgg catgcactcc cctctgctgt ccacaaacca cgaggatgtt |
| 91801 | ttcaaagggg aaaaggaagc actgaccact cccctccatg tggcagtatc tggagttcag |
| 91861 | agcccaggag tcctcgggca tgagctcatc tgattctcac tcacggcaca ggcagtgcag |
| 91921 | caaggcaagg ccctgagcag ggaaggccaa ggtcacaaag gagtcaggat ggagctggga |
| 91981 | ccagaagatc cctgctctcc ctccaggctg cccacagccc atcctccatt tcccttcctg |
| 92041 | cccagcaatc ccatggcaca ccacaggccc ccaccacatc ttctcRgagt ctctttccac |
| 92101 | ctggcctggt tccacaaact acaaatcagc aggtgctcca ccagcagtgg gactccagcc |
| 92161 | ccgcttgccc aaaccagaaa cagctggtgg tatagaagca gccagcgcag actttcttcc |
| 92221 | aaaacaagac ctcagccagc ggggcctcca cagcctgccc cagaggggcc ctggggctgc |
| 92281 | ccgctggaac cagaaaggcc cagtttgggg gcagaaaggg gcccagacag acggcaggtc |
| 92341 | aggcaacaac cacagaccgt gaagatggaa gtgatgctgc tgaaaccgaa ggagccaccc |
| 92401 | actagcaggc aagcacttgt gggtggcagg gaggcagtag tccccagggc tcaactctca |
| 92461 | gccttacccc agaaagagag gggtatgata aagactacat tcagaattaa atttacattc |
| 92521 | tagcttttgc tctgaaatgc tttggtctgt cagattatct tcccattagc agtgttctgg |
| 92581 | tggctgggta ggggggtggt cccagacccc ccaaatgata ttcacttgca tagccactaa |
| 92641 | catttgcaaa atacttcata gtgtcccaag cattgttttt ccatctgatt atcacaacag |
| 92701 | ccctgggaag aaggcagggc tgatacctgc ctgatggata aggaaactga agtccagctt |
| 92761 | ggggacttaa tcagcccaat ttaatgcctt tggtcactgg ctgcatcctg gggccagcta |
| 92821 | tccctgcaac cctggagcag gtgatgccct ccctggccca gagggaaaag tgtaagcaaa |
| 92881 | tctctggtac agggaggtgg aaggtcatcg aggccaacat ctacctcccc agcagaggtg |
| 92941 | agggctgcca gggatgctgg caggtgggct ggcagagggg gtatagggaa ggggtctcca |
| 93001 | ggaaagacgg cacagaacag aacgtcagag aggtctattc tagtgtctac tctgctgtgt |
| 93061 | atggaaccat gggctcattc cctcacatgc aaggggagga gcatgggcca cacagaaacc |
| 93121 | gggtgatggc taaagctggc ctcaggatca agaagaccca agtctcagga gctagcagca |
| 93181 | agctgaggaa agagagagag ccagcaccat ggaaacatcg ctgccttgga ggcaggaaga |
| 93241 | aaggtccaca cagggaaagt gctcaaggcc aaccttgcga acacgaggcc tgagaggggg |
| 93301 | cctcacctgc actagcctca gctctgtcca ccctgtgggc tgtgaaaccc tggagcagcc |
| 93361 | actcaccttt cctgagcctc aggtgcctca tctgtaaagc aggagtaata atagaaccta |
| 93421 | cctgatagaa gatcaaagaa attaaatgaa acaatatgtg tatagttgga agggaattct |
| 93481 | cactttctac tttaggcaat acttcctgat tatctgattt aacaagaaac acatctttgt |
| 93541 | tttataataa gatttccaaa ataataagag tatacagtaa ctagctcagc atttgacaca |
| 93601 | cagcaggtgc ttaataaaag gctgaggcaa ccccacaact gtgcccagct tatctaaggg |
| 93661 | ccaaggcttt ccccatttgt tctctccttt aatgggattt taggctcttc cgtcagctca |
| 93721 | gtaagctagg ttctggcctc cacttattct ctagtgacct gcatatcacc atttaactgt |
| 93781 | ggttaagcag ggtgcctgga cacaccatag ctagtgaaca tgtagctata agaagccctt |
| 93841 | gaaccacgat gccacactca ccaacaactg ggaatctgag cctcatttcc tctgctgtaa |
| 93901 | agggcaatat cctgcaattc cacccaagga tgaaaggaag tgagaYgcca tgatatcaca |
| 93961 | ttgcagacat aaacaaaaaa ctgtccacat atttgtgtta tgatgacctc cagtcctccc |
| 94021 | caaccaattt ccaggctggg gaaactgcag cctcaggtcc atccatcaaa ggccgggttg |
| 94081 | gatttttcag gcagtgaaac tactctgttc aatactgtaa tggtggacac ctgccattcc |
| 94141 | acatttgtcc aaacccatag aacatacacc accaagaggg aacactgatg taaaccatgg |
| 94201 | actccgggtg acagtggtgt caatgtagat tcatcaactg taaaaaatct accactctgg |
| 94261 | tggaggatgt tgataatggg ggaagctatg cctgttaggg gtgggggggg cgcagtatat |
| 94321 | gtaaaatctc tgtaccttct tctcaatttt gctgtgaacc taaaactgct ctataaaaaa |
| 94381 | taaggtcttt aatttaaaag agaaaaggac caggctgggc tctcctgatg gctgtcaggt |
| 94441 | tcacacatga gtgaggacac agcacatact tgagacagtg tgtattcaag gaccgcaccc |
| 94501 | tggcatcctc accaacaagg agcccagact taactcctcc atccttgcaa tcaactgagc |
| 94561 | aacaatgagg ctgcagacac agagtctcat atagtaacag ctggggcgcc agaactgtgg |
| 94621 | gatctagtca aggtacacta gggcgaacat cttaaccctc actaggcctc agttacctaa |
| 94681 | aaagagggag ctgcaaaaat agtaataaca tggggggttg tgcgggataa tcattaaggc |
| 94741 | cccttccagt tctaacattt caggatctga aatttattag cactactttc tttaaccttc |
| 94801 | taaccttaag aaggctccaa tccccagctc caatcccttc taaggcccag gtggatttcc |
| 94861 | ctaacccatc agtcctccca taagagcttt cataggtgct atcccctccc cgagggcagt |
| 94921 | cagagaccct cagctgacat ccgcagccct gtgccaggtc ctcccctcct gcctaacttg |
| 94981 | taccaggctc tggccaagcc tgatctgtgt tcctcaacat ggcacacatc ttcccatgtc |
| 95041 | cttcatcctc ccctcccgag tcacccacaa agaaggcccc actgtctcca ctcagcctcc |
| 95101 | gaggcccaga ttacacacca cccgctaaac acctgagcca gaattcacac tgcaaaagag |
| 95161 | gactggagag cacctaacta agctggagag agggtgacag ttcgttaggc gctaccgtat |
| 95221 | gccaggtgct gggccgcatc atgacgcaca tgacctcatt ttcttcttgc cacacagata |
| 95281 | tggaaattat catccacact ttccagcaag gaaaccagtc gacgcagcta catggatgag |
| 95341 | gaagcagact cagagaagtY aagtcatttg cctgagaata cacagctggt tagtggtaga |
| 95401 | acaaataatg cagcctcaat ctgtcagatt ccaacatcag tgccctttcc actgctctac |
| 95461 | actgcctgga gaggcggcag gctgaagctc agaggtggga aaccagacct actttccagt |
| 95521 | tctccagaac gtgccccaag ttcctagccc ctgcctttat atgtctctag ctttatatgc |
| 95581 | aaccaggtgg caatctgccc cacatcacct gcagtcaaca gggtttagaa tctcaggctg |
| 95641 | gaagggagct cagaggacat tccatcctgt gcttacccag tcctctgagg agcccaggga |
| 95701 | ctccagctgg ccaactttag caatccagat ctcaccaact tcctccccaa aacaattcct |
| 95761 | ggggcagcca gcactggcta cagttaggct gctcccagtt ctgtttccac tggcccttct |
| 95821 | ctcccctctg tggtcccctg gaataaggaa caagatgccc atcttcagca tgacagccag |
| 95881 | ccctgaggac atttggaacc tgcagccttc tctatactgt tccacatagg gactgccccc |
| 95941 | cctccagtcc cccaccaacc ctctacccca cactatctct agttggttct agcactggac |
| 96001 | ccaacattcc aggcatggtt tgcctgacca aatgcaggct ttcacctcca cccttttagg |
| 96061 | tagtatattc ctattgaggg agcctgggta tacatttgct tttcttacag ctgagtcacc |
| 96121 | tactgaagaa tgtcctttct caatgacaac aacctgcatg tatgcctaac tggtgagaac |
| 96181 | aaagttctca tgtagaaacg tgtgcttgag ggctgggtgc ggtggctcat gcctgtaatt |
| 96241 | ccagcacttt gggaggccga ggtgggcgga tcacctgagg tcaggagttc gagatcagcc |
| 96301 | tggccaacat ggcaaaaccc agtctctact aaaaatacaa aaattagcca ggtgtggtgg |
| 96361 | cgggtgcctg tagtcccagc tactggggag gctgaggtag gagaatcgct tgaacttggg |
| 96421 | aggtggagat tgcagtaagc cgagatcacg ccacagcact ccagcctggg agacagagtg |
| 96481 | ggactccatc tcaaaagaaa aaaaaaaaaa aaaaggggtg tttgaggagg tgtgagttac |
| 96541 | ctaagggggc agaagtaact gattgattca gtaatMccct atctaggaat ctagagatct |
| 96601 | ttgcatcagt acacagaagc tggacaaaaa tatgcactgc cacatgcttt gtaatagaca |
| 96661 | tttttgctat tcttgaatgt ccatcaagaa gggactggtt aaacaaacca ccattcattc |
| 96721 | Ratggaatac tatacagcca tcaaaaagaa tggggtagat tgcttggcaa tgatacagaa |
| 96781 | agatgcccat aaaatacaaa gcaaagtgca gatttatatg tgtaacagga tgctattttt |
| 96841 | ttcaaaaaca cattgtgtgt gtgtgtgtat acatacggaa tcttaaatgt gcatagaaaa |
| 96901 | aggcctggaa gcccagtgct ttgggaggcc aaggtgggag gattgcttga ggccaggggt |
| 96961 | tcaagaccag cctgggcaaa aagcaagaat agattctcta ttaaaaaaaa acttttttac |
| 97021 | attagcagag catggtggca catgcctgga gtcccagcta ctcggaaagt tgaggtggga |
| 97081 | ggatcacttg agcccagcag ttcaggctac agtgagctat gatcatgcca ctgaactcca |
| 97141 | gcctgagtga cagactaata ctctgtctct taaaaaaaag aaaagaagaa aagaaaaaag |
| 97201 | tctcaaagga tacataccaa actgctacta gtagactgct ctacagaagg acctcggtaa |
| 97261 | ggaactaaaa ccaaaagaaa cttttatttt acctatgcta ttttaactct ttacatggga |
| 97321 | aatctagtcc tgttcttata caattataaa attagttttt aaatacagta gggacagagg |
| 97381 | aaggggaaag gaaggagaga gaagacaact gtcatttttt acattatatc ttttgtactg |
| 97441 | cttgagtttt ggggggtttt tccccaataa tcatgtgcta tttggtaatt ttttttaaag |
| 97501 | ggaaatttta aaatattttt ctaaaaaatt taagtgagct gagtgcagtg gtgcatgcct |
| 97561 | gtagtcccag ctactccaga gacagacgca ggaggatggc ctgaaaccag agtttatcta |
| 97621 | accctgtaac tcccctgcct ccaagaggta agatgccaat tttggggaat tcctcttata |
| 97681 | caaaatatgg tggaaagagg gtttcacaga ttcaaatcct ggctatgatc ccttctcgaa |
| 97741 | tgaccctggg taggcctcac tttccugtt tctccctatg gaaatgttat gaggaccaag |
| 97801 | taggaaccat agagttctct gtaaacctta aaatgctacc cagacaggag cagtaatggc |
| 97861 | tgccaaggag ctaggaagga aaaggggctg gacaggggct gaggtactca gatgaaagcc |
| 97921 | aaagatgcct tgtgttaaca acaaaaagct gaaagctacc taaaatgaag agaaaaatga |
| 97981 | ataaatacat tgtggcaatt tcctatcagg aaatactata cagcagtgaa aaaagaatga |
| 98041 | gccacagctg gagatacgaa catgaatgaa tctgggtctc actctgttgc ccaggctgga |
| 98101 | gtgcagtggc atgatctcag ttcactgcaa cttccacctc ccagcccaag tcatcctccc |
| 98161 | acctLagcct ctcaagtagc tggaactaca ggcatgcgcc accacaccca gctaattttt |
| 98221 | gtattgtttg cagagatggg ttttggccat gttgcccaag ctggtctcaa actcctgggc |
| 98281 | tcaagagatc cacctgcctg ctctgcctcc caaagWgcca gtaaggcatg agtcaccacg |
| 98341 | ctcagcctaa ataacatttt taaacataaa aaaatactat gtattattta gggatgcaca |
| 98401 | cattaaaaaa aaaactttaa agaaatgcaa gaaaaaaacc ccactaaatt cagaacactt |
| 98461 | gtttcttgtt ggtggctgat gaaggagaaa tttcaaStga ttggaaatgt gttatttctt |
| 98521 | aaaccagatg agtgagcatg taggtgcctg cgtgttaccc tcggtatctg agagactcct |
| 98581 | tcaatttttc ttttttcttt ttactgtatt gtttgctttt tttctgcttc aacttttatt |
| 98641 | ttagattcgg ggggtacatg tgcaggtttg ctacctgggt atactgcgtg atttgggtag |
| 98701 | gggtgatccc atcacctggg tactgagcat agttcccaac agtttttcaa ctctgcctcc |
| 98761 | ctccctcccc actctagcag tccccagtgt ctactgttgc catctttatg tccatgagga |
| 98821 | cctgatattt agctccactt ataagtgaga aaatgcagca tttggttttc tgttcctgtg |
| 98881 | ttaatgtgct taggacaata ttttctaaaa gtctatgttg ccagtttcct gaggaccaga |
| 98941 | tcacgcagga gttgcacctg cagagacaga agtgggctgg agagggacca gggctcctaa |
| 99001 | gtctgctgct gaagctgact atagcgaagg caaaggacaa gtggcctgaa ggccaacaga |
| 99061 | gagcctggcc aatgctctgc ccagtgggtg gagactgagc caaaggccct tgactcatag |
| 99121 | ggcagtccca tacctgccct ggcctctcag catgactcct tggtgacaca actcccacag |
| 99181 | cacccacaca cctacaccca cctaccccac acacacaccc ctgcctgggc tgcccggttt |
| 99241 | gtccactgtg taggtgctca aggacgcaga gctcctgcct ctcaaggaac agcctcaggg |
| 99301 | tgaggtccct gggagagaag agttcagctc tgcccagagc agccctcaga caaacagcca |
| 99361 | gctgctgctc accagtcatc cRttccaatc tgaagagtga ctgtcctggg ccagtcatgg |
| 99421 | gaaaatcatc aagaacttcc cRcaatggtc atggtggccg ccaaggtctt tctctggggg |
| 99481 | ttgggagctg tgctccccca caaaacaagg ggacccagct gccctgtgcc ttcctgtggc |
| 99541 | aactctctgg aggatgcctg gataccctcc ctacagacgg ccagggatag gaggggcagg |
| 99601 | cactgcaggc atgtgggggc atagcagcaa gaatctagga gggcaggcac agcaaggcct |
| 99661 | ggcccagagg gaaaccagga gctgccagag ccaaagagag ggtgacccaa tcattggttg |
| 99721 | ttatagagtt tttaagactt ggaagagaat gccctgagat gccaNagagt gaaacatttt |
| 99781 | cattctaaag ctgggaaacc aaagtcggca aaggcgaaca gacgtgccca cagccacatg |
| 99841 | ggacagcagt ggcacagaag aaatgcacac ctcgtcctct ggtggccact gtgcactctc |
| 99901 | ttaccacacc atgctactgc tcaacctctc cccctccctc acagtggtca ggtaagcaat |
| 99961 | gagggaaaat gatggaggtg agaaaggaag aacccgggct |

Following is a CETP coding nucleotide sequence (cDNA, SEQ ID NO: 6). >gi|4557442|ref|NM_{—}000078.1| Homo sapiens cholesteryl ester transfer protein, plasma (CETP), mRNA.

| | |
|---|---|
| 1 | gtgaatctct ggggccagga agaccctgct gcccggaaga |
| | gcctcatgtt ccgtgggggc |
| 61 | tgggcggaca tacatatacg ggctccaggc tgaacggctc |
| | gggccactta cacaccactg |
| 121 | cctgataacc atgctggctg ccacagtcct gaccctggcc |
| | ctgctgggca atgcccatgc |
| 181 | ctgctccaaa ggcacctcgc acgaggcagg catcgtgtgc |
| | cgcatcacca agcctgccct |
| 241 | cctggtgttg aaccacgaga ctgccaaggt gatccagacc |
| | gccttccagc gagccagcta |
| 301 | cccagatatc acgggcgaga aggccatgat gctccttggc |
| | caagtcaagt atgggttgca |
| 361 | caacatccag atcagccact tgtccatcgc cagcagccag |
| | gtggagctgg tggaagccaa |
| 421 | gtccattgat gtctccattc agaacgtgtc tgtggtcttc |
| | aaggggaccc tgaagtatgg |
| 481 | ctacaccact gcctggtggc tgggtattga tcagtccatt |
| | gacttcgaga tcgactctgc |
| 541 | cattgacctc cagatcaaca cacagctgac ctgtgactct |
| | ggtagagtgc ggaccgatgc |
| 601 | ccctgactgc tacctgtctt tccataagct gctcctgcat |
| | ctccaagggg agcgagagcc |
| 661 | tgggtggatc aagcagctgt tcacaaattt catctccttc |
| | accctgaagc tggtcctgaa |
| 721 | gggacagatc tgcaaagaga tcaacgtcat ctctaacatc |
| | atggccgatt ttgtccagac |
| 781 | aagggctgcc agcatccttt cagatggaga cattggggtg |
| | gacatttccc tgacaggtga |
| 841 | tcccgtcatc acagcctcct acctggagtc ccatcacaag |
| | ggtcatttca tctacaagaa |
| 901 | tgtctcagag gacctccccc tccccacctt ctcgcccaca |
| | ctgctggggg actcccgcat |
| 961 | gctgtacttc tggttctctg agcgagtctt ccactcgctg |
| | gccaaggtag ctttccagga |
| 1021 | tggccgcctc atgctcagcc tgatgggaga cgagttcaag |
| | gcagtgctgg agacctgggg |
| 1081 | cttcaacacc aaccaggaaa tcttccaaga ggttgtcggc |
| | ggcttcccca gccaggccca |
| 1141 | agtcaccgtc cactgcctca agatgcccaa gatctcctgc |
| | caaaacaagg gagtcgtggt |
| 1201 | caattcttca gtgatggtga aattcctctt tccacgccca |
| | gaccagcaac attctgtagc |
| 1261 | ttacacattt gaagaggata tcgtgactac cgtccaggcc |
| | tcctattcta agaaaaagct |
| 1321 | cttcttaagc ctcttggatt tccagattac accaaagact |
| | gtttccaact tgactgagag |
| 1381 | cagctccgag tccatccaga gcttcctgca gtcaatgatc |
| | accgctgtgg gcatccctga |
| 1441 | ggtcatgtct cggctcgagg tagtgtttac agccctcatg |
| | aacagcaaag gcgtgagcct |
| 1501 | cttcgacatc atcaaccctg agattatcac tcgagatggc |
| | ttcctgctgc tgcagatgga |
| 1561 | ctttggcttc cctgagcacc tgctggtgga tttcctccag |
| | agcttgagct agaagtctcc |
| 1621 | aaggaggtcg ggatggggct tgtagcagaa ggcaagcacc |
| | aggctcacag ctggaaccct |
| 1681 | ggtgtctcct ccagcgtggt ggaagttggg ttaggagtac |
| | ggagatggag attggctccc |
| 1741 | aactcctccc tatcctaaag gcccactggc attaaagtgc |
| | tgtatccaag |

CDS: 131-1612 base pairs
Signal peptide: 131-181 base pairs
Mature peptide: 182-1609 base pairs
PolyA signal: 1771-1776 base pairs

Following is a second PROL4 cDNA sequence (SEQ ID NO:7). >gi|6005801|ref|NM007244.1| Homo sapiens proline rich 4 (lacrimal) (PRR4), mRNA

| | |
|---|---|
| 1 | cagagcctcc ttcaagatgc tgctggtcct gctctcagtg |
| | tccttctgg ctctgagctc |
| 61 | agctcagagc acagataatg atgtgaacta tgaagacttt |
| | actttcacca taccagatgt |
| 121 | agaggactca agtcagagac cagatcaggg accccagaga |
| | cctcctcctg aaggactcct |
| 181 | acctagaccc cctggtgata gtggtaacca agatgatggt |
| | cctcagcaga gaccaccaaa |
| 241 | accaggaggc catcaccgcc atcctccccc acctcctttt |
| | caaaatcagc aacgaccacc |
| 301 | ccaacgagga caccgtcaac tctctctacc ccgatttcct |
| | tctgtcagcc tgcaggaagc |
| 361 | atcatcattc ttccggaggg acagaccagc aagacatccc |
| | caggagcaac cactctggta |
| 421 | atctagaatt cagtggcaga aaataaataa gaagataact |
| | tcattcagaa agccatgaca |
| 481 | ttgaaataat gtggtcataa ctctttc |

CDS: 17 . . . 421 base pairs
PolyA signal: 442 . . . 447 base pairs

Following is a GRID2 cDNA sequence (SEQ ID NO: 8). >gi|4557632|ref|NM_{—}001510.1| Homo sapiens glutamate receptor, ionotropic, delta 2 (GRID2), mRNA.

| | |
|---|---|
| 1 | atggaagttt tccccttgct cttggttttg tccgtctggt |
| | ggtctcgaac ctgggactcg |
| 61 | gcgaatgcgg attcgatcat tcacatcgga gcaatttttg |
| | atgaatctgc caaaaaggat |
| 121 | gatgaggtat ttcgcactgc ggttggtgac cttaaccaga |
| | atgaagagat cttacagact |
| 181 | gagaaaatca cattttcagt gacgtttgtt gatggcaaca |
| | accctttcca agcagttcaa |
| 241 | gaagcctgtg aacttatgaa tcaaggcatc ttggccctgg |
| | tcagctccat tggctgcacg |
| 301 | tcagcaggat ccctccagtc tttggcagac gccatgcata |
| | tcccccacct cttcattcag |
| 361 | cgctcaacag ctgggacccc aaggagtggc tgtggactca |
| | cccggagcaa caggaatgat |
| 421 | gactacactc tctcagttcg cccacctgtc tacttgcatg |
| | atgttatcct aagagtggtc |
| 481 | acagagtatg cctggcagaa attcattata ttctatgata |
| | gtgaatacga tatccgtgga |
| 541 | atacaggagt tcttggacaa agtctctcag cagggaatgg |
| | atgttgcact tcagaaggta |
| 601 | gaaaacaaca tcaataaaat gattaccact ctctttgaca |
| | ccatgagaat agaagaactg |
| 661 | aatcgctatc gagacactct taggcgagcg atccttgtta |
| | tgaatcctgc tacagccaaa |
| 721 | tccttcatta ctgaggttgt ggagactaat ttggttgctt |
| | ttgactgtca ctggatcatt |
| 781 | ataaatgagg aaataaacga tgtggacgta caggaacttg |
| | taagaaggtc aattggaagg |
| 841 | ttaacgatta ttcggcagac atttccaatt ccccagaaca |
| | taagtcagcg gtgtttccgt |
| 901 | ggcaaccatc gaatatcttc aacattgtgt gatccaaagg |
| | atccatttgc tcagaatatg |
| 961 | gagatttcca acctttacat atatgacacg gtgcttctgc |
| | ttgctaatgc ttttcataag |
| 1021 | aagctggagg accgaaagtg gcacagcatg gcaagtctgt |
| | catgtatcag aaagaactca |
| 1081 | aagccctggc agggtgggcg ctccatgttg gagaccatca |
| | agaagggtgg agttagtggg |
| 1141 | ttgactggag agctagaatt tggagaaaat ggaggcaatc |
| | ccaatgtcca ctttgaaatc |
| 1201 | cttggaacca actatggaga agagcttggc agaggtgttc |
| | gaaaacttgg gtgctggaat |
| 1261 | cctgtcacag gtctgaatgg gtcactgact gacaagaaat |
| | tggagaataa catgcgtgga |
| 1321 | gtggttctac gtgtagtaac tgttctggaa gaaccttttg |
| | tgatggtctc tgaaaatgtc |
| 1381 | ttgtgtaagc cgaagaaata ccagggcttc tccattgatg |
| | ttttggatgc cttatctaac |
| 1441 | tacctgggtt ttaactacga aatttacgta gcaccggatc |
| | acaaatacgg aagcccacaa |
| 1501 | gaagatggga catggaatgg cttggtagga gaacttgtct |
| | ttaagagagc cgacataggg |
| 1561 | atttctgctt taaccatcac tccagatcgt gaaaatgtgg |
| | tggactttac gacacgttac |
| 1621 | atggactact cagtgggggt actacttcga agggctgaaa |
| | agacagtgga tatgtttgcc |
| 1681 | tgtcttgcac catttgatct ctctctatgg gcttgcattg |
| | ctggcacagt ccttctggtg |
| 1741 | ggtctactgg tctacctctt gaactggctt aatcccccac |
| | gattacaaat gggatcaatg |
| 1801 | acgtctacta ctctctacaa ctccatgtgg tttgtgtatg |
| | gatcttttgt acaacaaggc |
| 1861 | ggggaagtcc cgtacacgac tctggctacc cgaatgatga |
| | tgggggcttg gtggctattt |
| 1921 | gctttgattg ttatctcatc ttacacggca aacctcgctg |
| | ctttcctcac tattacacgc |
| 1981 | attgaaagtt ccatccagtc tctccaggac ctttccaagc |
| | aaacagaaat cccttatggc |
| 2041 | acagtcctag actctgcggt atatgagcat gtccgcatga |
| | aaggactgaa tccttttgag |
| 2101 | agggacagca tgtattccca aatgtggcgg atgatcaacc |
| | gaagcaatgg atcggagaac |
| 2161 | aatgttctgg agtcccaggc aggcattcaa aaggtaaaat |
| | atggaaatta tgctttcgta |
| 2221 | tgggatgcag ctgtattgga atatgtggct atctatgacc |
| | cagattgttc cttttacacc |
| 2281 | attggaaata ctgttgctga tcggggatat ggaattgcat |
| | tacaacatgg cagtccttac |
| 2341 | cgagatgttt tttcacaaag gatcctggag cttcagcaga |
| | atggtgacat ggacatcctg |
| 2401 | aagcacaaat ggtggcctaa gaatggccag tgtgacctgt |
| | actcgtcagt ggacacaaag |
| 2461 | cagaaaggag gcgccctgga cataaagagc tttgcagggg |
| | tcttttgtat cctggctgct |
| 2521 | ggaattgtcc tctcctgctt catagccatg ctggagacgt |
| | ggtggaacaa gaggaaaggc |
| 2581 | tcccgggttc catcaaaaga ggatgacaag gaaattgacc |
| | tggagcacct ccatagacgt |
| 2641 | gtaaatagct tgtgcacaga tgacgacagc ccccataaac |
| | agttttccac ctcgtcaatt |
| 2701 | gatttgaccc ctctggacat tgacactttg ccaacacgac |
| | aagcactgga gcaaatcagt |
| 2761 | gatttcagga acactcatat taccacaaca acctttatcc |
| | cagagcagat ccagactctt |
| 2821 | agccgcacac tgtcagctaa agctgcttct ggtttcactt |
| | ttggcaacgt gcctgagcac |
| 2881 | cgaactggcc cttttaggca cagggcacct aatgggggct |
| | ttttcaggag tcctataaaa |
| 2941 | acaatgtcat ctattcctta tcaaccaact cctaccctgg |
| | ggctcaatct gggtaatgat |
| 3001 | ccagaccgag gcacctccat atga |

Following is a PDE4D cDNA sequence (SEQ ID NO: 9). >gi|4636198|ref|_{—}006203.3| Homo sapiens phosphodiesterase 4D, cAMP-specific (phosphodiesterase E3 dunce homolog, Drosophila) (PDE4D), mRNA.

| | |
|---|---|
| 1 | ggaattcatc tgtaaaaatc actacatgta acgtaggaga |
| | caagaaaaat attaatgaca |
| 61 | gaagatctgc gaacatgatg cacgtgaata attttccctt |
| | tagaaggcat tcctggatat |
| 121 | gttttgatgt ggacaatggc acatctgcgg gacggagtcc |
| | cttggatccc atgaccagcc |
| 181 | caggatccgg gctaattctc caagcaaatt ttgtccacag |
| | tcaacgacgg gagtccttcc |
| 241 | tgtatcgatc cgacagcgat tatgacctct ctccaaagtc |
| | tatgtcccgg aactcctcca |
| 301 | ttgccagtga tatacacgga gatgacttga ttgtgactcc |
| | atttgctcag gtcttggcca |
| 361 | gtctgcgaac tgtacgaaac aactttgctg cattaactaa |
| | tttgcaagat cgagcaccta |
| 421 | gcaaaagatc acccatgtgc aaccaaccat ccatcaacaa |
| | agccaccata acagaggagg |
| 481 | cctaccagaa actggccagc gagaccctgg aggagctgga |
| | ctggtgtctg gaccagctag |
| 541 | agaccctaca gaccaggcac tccgtcagtg agatggcctc |
| | caacaagttt aaaaggatgc |
| 601 | ttaatcggga gctcacccat ctctctgaaa tgagtcggtc |
| | tggaaatcaa gtgtcagagt |
| 661 | ttatatcaaa cacattctta gataagcaac atgaagtgga |
| | aattccttct ccaactcaga |
| 721 | aggaaaagga gaaaaagaaa agaccaatgt ctcagatcag |
| | tggagtcaag aaattgatgc |
| 781 | acagctctag tctgactaat tcaagtatcc caaggtttgg |
| | agttaaaact gaacaagaag |
| 841 | atgtccttgc caaggaacta gaagatgtga acaaatgggg |
| | tcttcatgtt ttcagaatag |
| 901 | cagagttgtc tggtaaccgg cccttgactg ttatcatgca |
| | caccattttt caggaacggg |
| 961 | atttattaaa aacatttaaa attccagtag atactttaat |
| | tacatatctt atgactctcg |
| 1021 | aagaccatta ccatgctgat gtggcctatc acaacaatat |
| | ccatgctgca gatgttgtcc |
| 1081 | agtctactca tgtgctatta tctacacctg ctttggaggc |
| | tgtgtttaca gatttggaga |
| 1141 | ttcttgcagc aatttttgcc agtgcaatac atgatgtaga |
| | tcatcctggt gtgtccaatc |
| 1201 | aatttctgat caatacaaac tctgaacttg ccttgatgta |
| | caatgattcc tcagtcttag |
| 1261 | agaaccatca tttggctgtg ggctttaaat tgcttcagga |
| | agaaaactgt gacattttcc |
| 1321 | agaatttgac caaaaaacaa agacaatctt taaggaaaat |
| | ggtcattgac atcgtacttg |
| 1381 | caacagatat gtcaaaacac atgaatctac tggctgattt |
| | gaagactatg gttgaaacta |
| 1441 | agaaagtgac aagctctgga gttcttcttc ttgataatta |
| | ttccgatagg attcaggttc |
| 1501 | ttcagaatat ggtgcactgt gcagatctga gcaacccaac |
| | aaagcctctc cagctgtacc |
| 1561 | gccagtggac ggaccggata atggaggagt tcttccgcca |
| | aggagaccga gagagggaac |
| 1621 | gtggcatgga gataagcccc atgtgtgaca agcacaatgc |
| | ttccgtggaa aaatcacagg |
| 1681 | tgggcttcat agactatatt gttcatcccc tctgggagac |
| | atgggcagac ctcgtccacc |
| 1741 | ctgacgccca ggatattttg gacactttgg aggacaatcg |
| | tgaatggtac cagagcacaa |
| 1801 | tccctcagag cccctctcct gcacctgatg acccagagga |
| | gggccggcag ggtcaaactg |
| 1861 | agaaattcca gtttgaacta actttagagg aagatggtga |
| | gtcagacacg gaaaaggaca |
| 1921 | gtggcagtca agtggaagaa gacactagct gcagtgactc |
| | caagactctt tgtactcaag |
| 1981 | actcagagtc tactgaaatt ccccttgatg aacaggttga |
| | agaggaggca gtaggggaag |
| 2041 | aagaggaaag ccagcctgaa gcctgtgtca tagatgatcg |
| | ttctcctgac acgtaacagt |
| 2101 | gcaaaaactt tcatgccttt ttttttttta agtagaaaaa |
| | ttgtttccaa agtgcatgtc |
| 2161 | acatgccaca accacggtca cacctcactg tcatctgcca |
| | ggacgtttgt tgaacaaaac |
| 2221 | tgaccttgac tactcagtcc agcgctcagg aatatcgtaa |
| | ccagtttttt cacctccatg |
| 2281 | tcatccgagc aaggtggaca tcttcacgaa cagcgttttt |
| | aacaagattt cagcttggta |
| 2341 | gagctgacaa agcagataaa atctactcca aattattttc |
| | aagagagtgt gactcatcag |
| 2401 | gcagcccaaa agtttattgg acttggggtt tctattcctt |
| | tttatttgtt tgcaatattt |
| 2461 | tcagaagaaa ggcattgcac agagtgaact taatggacga |
| | agcaacaaat atgtcaagaa |
| 2521 | caggacatag cacgaatctg ttaccagtag gaggaggatg |
| | agccacagaa attgcataat |
| 2581 | tttctaattt caagtcttcc tgatacatga ctgaatagtg |
| | tggttcagtg agctgcactg |
| 2641 | acctctacat tttgtatgat atgtaaaaca gattttttgt |
| | agagcttact tttattatta |
| 2701 | aatgtattga ggtattatat ttaaaaaaaa ctatgttcag |
| | aacttcatct gccactggtt |
| 2761 | atttttttct aaggagtaac ttgcaagttt tcagtacaaa |
| | tctgtgctac actggataaa |
| 2821 | aatctaattt atgaatttta cttgcacctt atagttcata |
| | gcaattaact gatttgtagt |
| 2881 | gattcattgt ttgttttata taccaatgac ttccatattt |
| | taaaagagaa aaacaacttt |
| 2941 | atgttgcagg aaaccctttt tgtaagtctt tattatttac |
| | tttgcatttt gtttcactct |
| 3001 | ttccagataa gcagagttgc tcttcaccag tgtttttctt |
| | catgtgcaaa gtgactattt |
| 3061 | gttctataat acttttatgt gtgttatatc aaatgtgtct |
| | taagcttcat gcaaactcag |
| 3121 | tcatcagttc gtgttgtctg aagcaagtgg gaaatatata |
| | aatacccagt agctaaaatg |
| 3181 | gtcagtcttt tttagatgtt ttcctactta gtatctccta |
| | ataacgtttt gctgtgtcac |
| 3241 | tagatgttca tttcacaagt gcatgtcttt ctaataatcc |
| | acacatttca tgctctaata |
| 3301 | atccacacat ttcatgctca tttttattgt ttttacagcc |
| | agttatagca agaaaaaggt |
| 3361 | ttttcccctt gtgctgcttt ataatttagc gtgtgtctga |
| | accttatcca tgtttgctag |
| 3421 | atgaggtctt gtcaaatata tcactaccat tgtcaccggt |
| | gaaaagaaac aggtagttaa |
| 3481 | gttagggtta acattcattt caaccacgag gttgtatatc |
| | atgactagct tttactcttg |
| 3541 | gtttacagag aaaagttaaa caaccaacta ggcagttttt |
| | aagaatatta acaatatatt |
| 3601 | aacaaacacc aatacaacta atcctatttg gttttaatga |
| | tttcaccatg ggattaagaa |
| 3661 | ctatatcagg aacatccctg agaaacggct ttaagtgtag |
| | caactactct tccttaatgg |
| 3721 | acagccacat aacgtgtagg aagtccttta tcacttatcc |
| | tcgatccata agcatatctt |
| 3781 | gcagagggga actacttctt taaacacatg gagggaaaga |
| | agatgatgcc actggcacca |
| 3841 | gagggttagt actgtgatgc atcctaaaat atttattata |
| | ttggtaaaaa ttctggttaa |
| 3901 | ataaaaaatt agagatcact cttggctgat ttcagcacca |
| | ggaactgtat tacagtttta |
| 3961 | gagattaatt cctagtgttt acctgattat agcagttggc |
| | atcatggggc atttaattct |
| 4021 | gactttatcc ccacgtcagc cttaataaag tcttctttac |
| | cttctctatg aagactttaa |
| 4081 | agcccaaata atcatttttc acattgatat tcaagaattg |
| | agatagatag aagccaaagt |
| 4141 | gggtatctga caagtggaaa atcaaacgtt taagaagaat |
| | tacaactctg aaaagcattt |
| 4201 | atatgtggaa cttctcaagg agcctcctgg ggactggaaa |
| | gtaagtcatc agccaggcaa |
| 4261 | atgactcatg ctgaagagag tccccatttc agtcccctga |
| | gatctagctg atgcttagat |
| 4321 | cctttgaaat aaaaattatg tctttataac tctgatcttt |
| | tacataaagc agaagaggaa |
| 4381 | tcaactagtt aattgcaagg tttctactct gtttcctctg |
| | taaagatcag atggtaatct |
| 4441 | ttcaaataag aaaaaaataa agacgtatgt ttgaccaagt |
| | agtttcacaa gaatatttgg |
| 4501 | gaacttgttt cttttaattt tatttgtccc tgagtgaagt |
| | ctagaaagaa aggtaaagag |
| 4561 | tctagagttt attcctcttt ccaaaacatt ctcattcctc |
| | tcctccctac acttagtatt |
| 4621 | tcccccacag agtgcctaga atcttaataa tgaataaaat |
| | aaaaagcagc aatatgtcat |
| 4681 | taacaaatcc agacctgaaa gggtaaaggg tttataactg |
| | cactaataaa gagaggctct |
| 4741 | ttttttttct tccagtttgt tggtttttaa tggtaccgtg |
| | ttgtaaagat acccactaat |
| 4801 | ggacaatcaa attgcagaaa aggctcaata tccaagagac |
| | agggactaat gcactgtaca |
| 4861 | atctgcttat ccttgccctt ctctcttgcc aaagtgtgct |
| | tcagaaatat atactgcttt |
| 4921 | aaaaaagaat aaaagaatat ccttttacaa gtggctttac |
| | atttcctaaa atgccataag |
| 4981 | aaaatgcaat atctgggtac tgtatgggga aaaaaatgtc |
| | caagtttgtg taaaaccagt |
| 5041 | gcatttcagc ttgcaagtta ctgaacacaa taatgctgtt |
| | ttaattttgt tttatatcag |
| 5101 | ttaaaattca caataatgta gatagaacaa attacagaca |
| | aggaaagaaa aaacttgaat |
| 5161 | gaaatggatt ttacagaaag ctttatgata atttttgaat |
| | gcattattta ttttttgtgc |
| 5221 | catgcatttt ttttctcacc aaatgacctt acctgtaata |
| | cagtcttgtt tgtctgttta |
| 5281 | caaccatgta tttattgcaa tgtacatact gtaatgttaa |
| | ttgtaaatta tctgttctta |
| 5341 | ttaaaacatc atcccatgat ggggtggtgt tgatatattt |
| | ggaaactctt ggtgagagaa |
| 5401 | tgaatggtgt gtatacatac tctgtacatt tttcttttct |
| | cctgtaatat agtcttgtca |
| 5461 | ccttagagct tgtttatgga agattcaaga aaactataaa |
| | atacttaaag atatataaat |
| 5521 | ttaaaaaaac atagctgcag gtctttggtc ccagggctgt |
| | gccttaactt taaccaatat |
| 5581 | tttcttctgt tttgctgcat ttgaaaggta acagtggagc |
| | tagggctggg cattttacat |
| 5641 | ccaggctttt aattgattag aattctgcca ataggtggat |
| | tttacaaaac cacagacaac |
| 5701 | ctctgaaaga ttctgagacc cttttgagac agaagctctt |
| | aagtacttct tgccagggag |
| 5761 | cagcactgca tgtgtgatgg ttgtttgcca tctgttgatc |
| | aggaactact tcagctactt |
| 5821 | gcatttgatt atttcctttt tttttttttt taactcggaa |
| | acacaactgg gggaat |

Following is a GPX3 cDNA sequence (SEQ ID NO: 10). >gi|6006000|ref|NM_{—}002084.2| Homo sapiens glutathione peroxidase 3 (plasma) (GPX3), mRNA.

| | |
|---|---|
| 1 | agccaaaaga ggaagggacc ggcctcccac gtccacaggg |
| | acctgacttc cacctctctg |
| 61 | cccagatttg cttatgtcac tgtcgccccg ggacggggag |
| | gtggggagct gagggcaagt |
| 121 | cgcgcccgcc cctgaaatcc cagccgccta gcgattggct |
| | gcaagggtct cggcttggcc |
| 181 | gcggattaat cacacccgag ggcttgaaag gtggctggga |
| | gcgccggaca cctcagacgg |
| 241 | acggtggcca gggatcaggc agcggctcag gcgaccctga |
| | gtgtgccccc accccgccat |
| 301 | ggcccggctg ctgcaggcgt cctgcctgct ttccctgctc |
| | ctggccggct tcgtctcgca |
| 361 | gagccgggga caagagaagt cgaagatgga ctgccatggt |
| | ggcataagtg gcaccattta |
| 421 | cgagtacgga gccctcacca ttgatgggga ggagtacatc |
| | cccttcaagc agtatgctgg |
| 481 | caaatacgtc ctctttgtca acgtggccag ctactgaggc |
| | ctgacgggcc agtacattga |
| 541 | actgaatgca ctacaggaag agcttgcacc attcggtctg |
| | gtcattctgg gctttccctg |
| 601 | caaccaattt ggaaaacagg aaccaggaga gaactcagag |
| | atccttccta ccctcaagta |
| 661 | tgtccgacca ggtggaggct ttgtccctaa tttccagctc |
| | tttgagaaag gggatgtcaa |
| 721 | tggagagaaa gagcagaaat tctacacttt cctaaagaac |
| | tcctgtcctc ccacctcgga |
| 781 | gctcctgggt acatctgacc gcctcttctg ggaacccatg |
| | aaggttcacg acatccgctg |
| 841 | gaactttgag aagttcctgg tggggccaga tggtataccc |
| | atcatgcgct ggcaccaccg |
| 901 | gaccacggtc agcaacgtca agatggacat cctgtcctac |
| | atgaggcggc aggcagccct |
| 961 | gggggtcaag aggaagtaac tgaaggccgt ctcatcccat |
| | gtccaccatg taggggaggg |
| 1021 | actttgttca ggaagaaatc cgtgtctcca accacactat |
| | ctacccatca cagacccctt |
| 1081 | tcctatcact caaggcccca gcctggcaca aatggatgca |
| | tacagttctg tgtactgcca |
| 1141 | ggcatgtggg tgtgggtgca atgtgggtgt ttacacacat |
| | gcctacaggt atgcgtgatt |
| 1201 | gtgtgtgtgt gcatgggtgt acagccacgt gtctacctat |
| | gtgtctttct gggaatgtgt |
| 1261 | accatctgtg tgcctgcagc tgtgtagtgc tggacagtga |
| | caaccctttc tctccagttc |
| 1321 | tccactccaa tgataatagt tcacttatac ctaaacccaa |
| | aggaaaaacc agctctaggt |
| 1381 | ccaattgttc tgctctaact gatacctcaa ccttggggcc |
| | agcatctccc actgcctcca |
| 1441 | aatattagta actatgactg acgtccccag aagtttctgg |
| | gtctaccaca ctccccaacc |
| 1501 | ccccactcct acttcctgaa gggccctccc aaggctacat |
| | ccccacccca cagttctccc |
| 1561 | tgagagagat caacctccct gagatcaacc aaggcagatg |
| | tgacagcaag ggccacggac |
| 1621 | cccatggcag gggtggcgtc ttcatgaggg aggggcccaa |
| | agcccttgtg ggcggacctc |
| 1681 | ccctgagcct gtctgagggg ccagccctta gtgcattcag |
| | gctaaggccc ctgggcaggg |
| 1741 | atgccacccc tgctccttcg gaggacgtgc cctcacccct |
| | cactggtcca ctggcttgag |
| 1801 | actcaccccg tctgcccagt aaaagccttt ctgcagcaaa |
| | aaaaaaaaaa aaaaaa |

CDS: 299-979 base pairs
Signal peptide: 299-358 base pairs
Mature peptide: 359-976 base pairs
PolyA signal: 1838 base pairs

Following is a CETP amino acid sequence (SEQ ID NO: 11). >gi|4557443|ref_NP900069.1| cholesteryl ester transfer protein, plasma precursor [Homo sapiens]

MLAATVLTLALLGNAHACSKGTSHEAGIVCRITKPALLVLNHETAKVIQT
AFQRASYPDITGEKAMMLLGQVKYGLHNIQISHLSIASSQVELVEAKSID
VSIQNVSVVFKGTLKYGYTTAWWLGIDQSIDFEIDSAIDLQINTQLTCDS
GRVRTDAPDCYLSFHKLLLHLQGEREPGWIKQLFTNFISFTLKLVLKGQI
CKEINVISNIMADFVQTRAASILSDGDIGVDISLTGDPVITASYLESHHK
GHFIYKNVSEDLPLPTFSPTLLGDSRMLYFWFSERVFHSLAKVAFQDGRL
MLSLMGDEFKAVLETWGFNTNQEIFQEVVGGFPSQAQVTVHCLKMPKISC
QNKGVVVNSSVMVKFLFPRPDQQHSVAYTFEEDIVTTVQASYSKKKLFLS
LLDFQITPKTVSNLTESSSESIQSFLQSMITAVGIPEVMSRLEVVFTALM
NSKGVSLFDIINPEIITRDGFLLLQMDFGFPEHLLVDFLQSLS
Protein: 1 . . . 493 amino acids
Signal peptide: 1-17 amino acids
Mature peptide: 18-493 amino acids

Following is a second PROL4 amino acid sequence (SEQ ID NO: 12). >gi|6005802|ref|NP_{—}009175.1| proline rich 4 (lacrimal) [Homo sapiens].

MLLVLLSVVLLAISSAQSTDNDVNYEDFTFTIPDVEDSSQRPDQGPQRPP
PEGLLPRPPGDSGNQDDGPQQRPPKPGGHHRHPPPPPFQNQQRPPQRGHR
QLSLPRFPSVSLQEASSFFRRDRPARHPQEQPLW
Protein: 1 . . . 134 amino acids
Signal peptide: 1-16 amino acids
Mature peptide: 17-134 amino acids

Following is a GRID2 amino acid sequence (SEQ ID NO: 13). >gi|4557633|ref|NP_{—}001501.1| glutamate receptor, ionotropic, delta 2 [Homo sapiens].

MEVFPLLLVLSVWWSRTWDSANADSIIHIGAIFDESAKKDDEVFRTAVGD
LNQNEEILQTEKITFSVTFVDGNNPFQAVQEACELMNQGILALVSSIGCT
SAGSLQSLADAMHIPHLFIQRSTAGTPRSGCGLTRSNRNDDYTLSVRPPV
YLHDVILRVVTEYAWQKFIIFYDSEYDIRGIQEFLDKVSQQGMDVALQKV
ENNINKMITTLFDTMRIEELNRYRDTLRRAILVMNPATAKSFITEVVETN
LVAFDCHWIIINEEINDVDVQELVRRSIGRLTIIRQTFPIPQNISQRCFR
GNHRISSTLCDPKDPFAQNMEISNLYIYDTVLLLANAFHKKLEDRKWHSM
ASLSCIRKNSKPWQGGRSMLETIKKGGVSGLTGELEFGENGGNPNVHFEI
LGTNYGEELGRGVRKLGCWNPVTGLNGSLTDKKLENNMRGVVLRVVTVLE
EPFVMVSENVLCKPKKYQGFSIDVLDALSNYLGFNYEIYVAPDHKYGSPQ
EDGTWNGLVGELVFKRADIGISALTITPDRENVVDFTTRYMDYSVGVLLR
RAEKTVDMFACLAPFDLSLWACIAGTVLLVGLLVYLLNWLNPPRLQMGSM
TSTTLYNSMWFVYGSFVQQGGEVPYTTLATRMMMGAWWLFALIVISSYTA
NLAAFLTITRIESSIQSLQDLSKQTEIPYGTVLDSAVYEHVRMKGLNPFE
RDSMYSQMWRMINRSNGSENNVLESQAGIQKVKYGNYAFVWDAAVLEYVA
IYDPDCSFYTIGNTVADRGYGIALQHGSPYRDVFSQRILELQQNGDMDIL
KHKWWPKNGQCDLYSSVDTKQKGGALDIKSFAGVFCILAAGIVLSCFIAM
IETWWNKRKGSRVPSKEDDKEIDLEHLHRRVNSLCTDDDSPHKQFSTSSI
DLTPLDIDTLPTRQALEQISDFRNTHITTTTFIPEQIQTLSRTLSAKAAS
GFTFGNVPEHRTGPFRHRAPNGGFFRSPIKTMSSIPYQPTPTLGLNLGND
PDRGTSI

Following is a PDE4D amino acid sequence (SEQ ID NO: 14). >gi|32306513|ref|NP_{—}006194.2| cAMP-specific phosphodiesterase 4D [Homo sapiens]

MMHVNNFPFRRHSWICFDVDNGTSAGRSPLDPMTSPGSGLILQANFVHSQ
RRESFLYRSDSDYDLSPKSMSRNSSIASDIHGDDLIVTPFAQVLASLRTV
RNNFAALTNLQDRAPSKRSPMCNQPSINKATITEEAYQKLASETLEELDW
CLDQLETLQTRHSVSEMASNKFKRMLNRELTHLSEMSRSGNQVSEFISNT
FLDKQHEVEIPSPTQKEKEKKKRPMSQISGVKKLMHSSSLTNSSIPRFGV
KTEQEDVLAKELEDVNKWGLHVFRIAELSGNRPLTVIMHTIFQERDLLKT
FKIPVDTLITYLMTLEDHYHADVAYHNNIHAADVVQSTHVLLSTPALEAV
FTDLEILAAIFASAIHDVDHPGVSNQFLINTNSELALMYNDSSVLENHHL
AVGFKLLQEENCDIFQNLTKKQRQSLRKMVIDIVLATDMSKHMNLLADLK
TMVETKKVTSSGVLLLDNYSDRIQVLQNMVHCADLSNPTKPLQLYRQWTD
RIMEEFFRQGDRERERGMEISPMCDKHNASVEKSQVGFIDYIVHPLWETW
ADLVHPDAQDILDTLEDNREWYQSTIPQSPSPAPDDPEEGRQGQTEKFQF
ELTLEEDGESDTEKDSGSQVEEDTSCSDSKTLCTQDSESTEIPLDEQVEE
EAVGEEEESQPEACVIDDRSPDT

Following is a GPX3 amino acid sequence (SEQ ID NO: IS). >gi|6006001|ref|NP_{—}002075.2| plasma glutathione peroxidase 3 precursor [Homo sapiens]

MARLLQASCLLSLLLAGFVSQSRGQEKSKMDCHGGISGTIYEYGALTIDG
EEYIPFKQYAGKYVLFVNVASYUGLTGQYIELNALQEELAPFGLVILGFP
CNQFGKQEPGENSEILPTLKYVRPGGGFVPNFQLFEKGDVNGEKEQKFYT
FLKNSCPPTSELLGTSDRLFWEPMKVHDIRWNFEKFLVGPDGIPIMRWHH
RTTVSNVKMDILSYMRRQAALGVKRK

Modifications may be made to the foregoing without departing from the basic aspects of the invention. Although the invention has been described in substantial detail with reference to one or more specific embodiments, those of skill in the art will recognize that changes may be made to the embodiments specifically disclosed in this application, yet these modifications and improvements are within the scope and spirit of the invention, as set forth in the claims which follow. Also, citation of the above publications or documents is not intended as an admission that any of the foregoing is pertinent prior art, nor does it constitute any admission as to the contents or date of these publications or documents. The content of each patent, patent application and other publication and document referenced is incorporated herein by reference in its entirety, including drawings, tables and cited documents.

1. A method for identifying a subject at risk of low bone mineral density (BMD), which comprises detecting the presence or absence of one or more polymorphic variations associated with low BMD in a nucleic acid sample from a subject, wherein the one or more polymorphic variations are detected in a nucleotide sequence in SEQ ID NOs:1-5, a substantially identical sequence thereof or a fragment of the foregoing; whereby the presence of the polymorphic variation is indicative of the subject being at risk of low BMD.

2. The method of claim 1, which further comprises obtaining the nucleic acid sample from the subject.

3. The method of claim 1, wherein the one or more polymorphic variations are detected at one or more positions selected from the group consisting of rs7500979, rs2217332, rs8044804, rs2270835, rs2133783, rs247609, rs952440, rs881598, rs2291955, rs2518054, rs866038, rs1436425, rs173537, rs247611, rs166017, rs173538, rs193694, rs7205692, rs8048746, rs247618, rs183130, rs6499863, rs4783961, rs3816117, rs711752, rs708272, rs1864163, rs4369653, rs1864165, rs891141, rs891143, rs7205804, rs5885, rs1532625, rs1532624, rs289712, rs7499892, rs5883, rs289714, rs158480, rs289717, rs4344729, rs289718, rs289719, rs2033254, rs4784744, rs291044, rs8053613, rs5881, rs5880, rs7198026, rs5882, rs8045701, rs289741, rs1801706, rs289742, rs289743, rs289746, rs172337, rs289747, rs1566439, rs7205459, rs289749, rs289751, rs8059220, rs8058353, rs289735, rs289737, rs291042, rs1875236, rs821466, rs821465, rs4275846, rs289707, rs821463, rs289706, rs1167741, rs2052880, rs1167742, rs1183256, rs1651665, rs1651666, rs4784751, rs1651667, rs8052091, rs1684574, rs1684575, rs1672865, rs821470, rs1549669, rs291040 and rs289754.

4. The method of claim 1, wherein the one or more polymorphic variations are detected at one or more positions selected from the group consisting of rs166017, rs193694, rs7205804, rs1801706, rs7205459 and rs821465.

5. The method of claim 1, wherein the one or more polymorphic variations are detected in a region spanning positions 14,328 to 68,805 in SEQ ID NO: 1.

6. The method of claim 1, wherein the one or more polymorphic variations are detected at one or more positions selected from the group consisting of rs523051, rs693620, rs2588349, rs2588350, rs619381, rs3759252, rs3759251, rs2418107, rs7303054, rs1838345, rs620878, rs2537817, rs1548803, rs667123, rs1838346, rs2159903, rs3944035, rs3741845, rs2110096, rs759055, rs589377, rs7960194, rs7978242, rs601051, rs4262797, rs2215714, rs1373434, rs2215715, rs612456, rs612808, rs689118, rs597468, rs592864, rs640372, rs7966559, rs654834, rs4763216, rs668521, rs669503, rs3906864, rs3906863, rs7957888, rs9300230, rs7306214, rs763839, rs2418105, rs666841, rs3851578, rs7138797, rs7295252, rs2418106, rs7299578, rs621112, rs3863320, rs1373432, rs1047699, rs1063193, rs2232959, rs2227296, rs1548804, rs2232958, rs2232957, rs2232956, rs1972571, rs3759250, rs3759249, rs1541525, rs2098248, rs2900550, rs7302130, rs4763583, rs4360778, rs1607695, rs1607694, rs2192139, rs7978300, rs7397871, rs4763217, rs2159900, rs10772370, rs7398682, rs2900551, rs2900552, rs2418214, rs2418215, rs965243, rs1117548, rs1520225, rs1520226, rs1520227, rs971919, rs2159901, rs2159902, rs2110099, rs7314847, rs7296003, rs4281556, rs4763219, rs3851579, rs3851580, rs1049119, rs2298866, rs2298865, rs2298864, rs2298863, rs3180393, rs2070837, rs7956204, rs2418216, rs3741844, rs4262798, rs2418217, rs2418218, rs7137492, rs2110100, rs1013312, rs4579993, rs1013313, rs7397106, rs2215716, rs2192140, rs4763589, rs1468697, rs2070837, rs3180393 and rs2298865.

7. The method of claim 1, wherein the one or more polymorphic variations are detected at one or more positions selected from the group consisting of rs2588350, rs619381, rs620878, rs759055, rs4262797, rs612808, rs3906863, rs7957888, rs763839, rs2418105, rs666841, rs3851578, rs7299578, rs621112, rs1047699, rs1548804, rs2232956, rs1520227 and rs2215716.

8. The method of claim 1, wherein the one or more polymorphic variations are detected in a region spanning positions 2,424 to 93,715 in SEQ ID NO: 2.

9. The method of claim 1, wherein the one or more polymorphic variations are detected at one or more positions selected from the group consisting of rs1433661, rs1485009, rs7681947, rs1816432, rs1485018, rs1485017, rs7438397, rs6834311, rs1368717, rs1017391, rs2870701, rs7679839, rs1385404, rs1368716, rs4693316, rs1905707, rs1905708, rs1905709, rs3912442, rs2082553, rs6831638, rs5860329, rs2870702, rs2870703, rs1948016, rs6835836, rs1994253, rs1905710, rs1485019, rs978191, rs1385405, rs7694361, rs1905711, rs1905734, rs1485012, rs1485013, rs4692981, rs7670552, rs7670932, rs7688091, rs7440540, rs2171000, rs2870704, rs7655758, rs7661436, rs7662289, rs7667044, rs7691929, rs5860330, rs901013, rs901012, rs901011, rs1948018, rs2870705, rs1948017, rs1905733, rs1385408, rs1385409, rs1385410, rs1485026, rs1485027, rs2904483, rs1385406, rs1905732, rs2046418, rs2200377, rs1905731, rs1905730, rs975713, rs6820985, rs7670441, rs6810794, rs7676623, rs1154861, rs1032125, rs1485022, rs1485024, rs3913651, rs4693319, rs1872383, rs2200376, rs7668090, rs7692930, rs967096, rs6822249, rs6532405, rs1017897, rs7672674, rs7694568, rs2904484, rs7340830, rs1485033, rs2870706, rs1905729, rs4693320, rs6848749, rs6532406, rs6532407, rs1905728, rs6819866, rs1905727, rs7674069, rs1905724, rs1905723, rs1485020 and rs6814101.

10. The method of claim 1, wherein the one or more polymorphic variations are detected at one or more positions selected from the group consisting of rs1433661, rs7679839, rs1368716, rs1905707, rs1905708, rs1994253, rs1485019, rs1905734, rs1485012, rs7670552, rs7691929, rs1948018, rs1948017, rs1485024, rs7694568, rs4693320, rs6848749, rs6532406, rs6532407 and rs6819866.

11. The method of claim 1, wherein the one or more polymorphic variations are detected in a region spanning positions 206 to 90,969 in SEQ ID NO: 3.

12. The method of claim 1, wherein the one or more polymorphic variations are detected at one or more positions selected from the group consisting of rs6886495, rs6450498, rs1472456, rs4700315, rs4700316, rs7714708, rs7710479, rs2968013, rs2968014, rs2968015, rs1391648, rs2055297, rs2055296, rs3989138, rs4700317, rs2036220, rs7727206, rs7723432, rs1546221, rs4479801, rs4395595, rs4395596, rs4699932, rs2936201, rs7356672, rs2936200, rs1909296, rs7703131, rs7445308, rs3087748, rs4321723, rs2968016, rs5868151, rs1874858, rs1874857, rs7712922, rs4631140, rs4469166, rs1078369, rs1078368, rs2968006, rs2968005, rs2936190, rs2409613, rs4415048, rs2968004, rs2968003, rs2968002, rs2936191, rs1498610, rs6874662, rs3060393, rs7729722, rs7733884, rs7714489, rs7735570, rs2936193, rs2291851, rs2291852, rs1498602, rs1995166, rs1498603, rs1498604, rs1498605, rs1948651, rs4699934, rs4700319, rs2279737, rs7720361, rs7706419, rs1006431, rs1353747, rs1498606, rs1353748, rs1553113, rs2968012, rs2968011, rs1498608, rs2936189, rs1498609, rs2968019, rs6891238, rs2968010, rs2968009, rs2936203, rs1498601, rs1498600, rs1498599, rs2936202, rs7730070, rs6450501, rs6450502, rs6889456, rs6894618, rs7706044, rs7707541, rs7712076, rs6892860, rs6867053, rs7737269, rs6864156, rs950447, rs2936196, rs7719347, rs1391649, rs1391650, rs1391651, rs1353749, rs10682149, rs5868153, rs1363882, rs2409626, rs2968018, rs954740, rs986067, rs6869400, and rs5010782.

13. The method of claim 1, wherein the one or more polymorphic variations are detected at one or more positions selected from the group consisting of rs7714708, rs1498602, rs4699934, rs1006431, rs1353747, rs1498608, rs1498609, rs2968010, rs2936202 and rs1391649.

14. The method of claim 1, wherein the one or more polymorphic variations are detected in a region spanning positions 1,599 to 82,591 in SEQ ID NO: 4.

15. The method of claim 1, wherein the one or more polymorphic variations are detected at one or more positions selected from the group consisting of rs1478398, rs1478397, rs1160114, rs1160113, rs1382323, rs1160112, rs7709870, rs7710643, rs7730467, rs6579829, rs6579830, rs6579831, rs6896232, rs1351131, rs1038074, rs1478396, rs6880512, rs4958858, rs4958431, rs4958432, rs6898463, rs4958859, rs4130064, rs4130065, rs4133119, rs4958860, rs4958861, rs4437356, rs4958868, rs1478400, rs6889375, rs1600159, rs6875892, rs4608909, rs2345000, rs4516840, rs2054440, rs707141, rs707142, rs841236, rs707143, rs707144, rs6869405, rs707145, rs707146, rs707148, rs707150, rs5872184, rs3763015, rs2042235, rs3763013, rs2042236, rs1946234, rs1946235, rs1946236, rs8177402, rs8177403, rs8177404, rs8177405, rs8177406, rs8177407, rs8177408, rs8177409, rs6888961, rs8177410, rs8177411, rs8177412, rs8177413, rs870407, rs870406, rs6873202, rs8177414, rs8177415, rs3805435, rs8177416, rs3792799, rs3792798, rs3828599, rs8177417, rs3792797, rs8177418, rs8177419, rs8177420, rs8177421, rs4958872, rs3792796, rs8177422, rs8177423, rs4958434, rs8177424, rs8177425, rs8177426, rs8177427, rs8177429, rs6889737, rs3792795, rs8177430, rs8177431, rs4958873, rs8177432, rs8177433, rs8177434, rs8177435, rs3763011, rs8177436, rs8177437, rs4958874, rs8177439, rs8177440, rs8177441, rs8177442, rs8177443, rs869975, rs869976, rs8177444, rs8177445, rs7721469, rs8177446, rs7704191, rs8177447, rs11548, rs2230303, rs7722386, rs8177448, rs8177449, rs2070593, rs8177450, rs8177451, rs8177452, rs8177453, rs8177454, rs3763010, rs8177455, rs8177456, rs736775, rs2277940, rs8177458, rs8177834, rs3924, rs2233312, rs2233311, rs2233310, rs2233309, rs4958875, rs2233308, rs2233307, rs2233306, rs2233305, rs2233304, rs2233303, rs2233302, rs2287719, rs2287720, rs7727034, rs7727250, rs7709800, rs3840312, rs2287721, rs6875293, rs3805434, rs2080982, rs2080983, rs2287722, rs2233301, rs2233300, rs4958876, rs2233299, rs2233298, rs2287723, rs2161359, rs7734456, rs4292439, rs4958878, rs6862024, rs3834819, rs2233297, rs2233296, rs2233295, rs2233294, rs7713028, rs7713223, rs7713567, rs888989, rs2233293, rs3749657, rs2233292, rs2112635, rs871269, rs3792794, rs6579837, rs3805433, rs5872186, rs2233291, rs2233290, rs2233289, rs4958435, rs4958880, rs1422673, rs2042234, rs3805432, rs3805431, rs2233288, rs2233287, rs3815720, rs3792792, rs3792791, rs2303018, rs3792790, rs4958436, rs2233286, rs2233285, rs7732451, rs2233284, rs1422674, rs3792789, rs4562032, rs6865077, rs1559126, rs3792788, rs1559127, rs3792786, rs6880110, rs6861227, rs3805430, rs1862364, rs4958881, rs3792785, rs6869605, rs6870205, rs4246047, rs4958882, rs3792784, rs3792783 and rs5872188.

16. The method of claim 1, wherein the one or more polymorphic variations are detected at one or more positions selected from the group consisting of rs1478398, rs1160114, rs1160113, rs1160112, rs4958858, rs4958431, rs6898463, rs4958859, rs4958860, rs4608909, rs707144, rs2042235, rs3763013, rs2042236, rs8177404, rs8177426, rs8177427, rs8177429, rs3792795, rs4958873, rs8177437, rs869975, rs8177447, rs11548, rs2277940, rs8177834, rs2233311, rs2233302, rs7727034, rs7727250, rs3805434, rs7734456, rs7713028, rs7713223, rs888989, rs3792794, rs4958880, rs1422673, rs3805432 and rs4958436.

17. The method of claim 1, wherein the one or more polymorphic variations are detected in a region spanning positions 231 to 86,539 in SEQ ID NO: 5.

18. The method of claim 1, wherein the one or more polymorphic variations are detected at one or more positions in linkage disequilibrium with one or more positions selected from the grout consisting of rs7500979, rs2217332, rs8044804, rs2270835, rs2133783, rs247609, rs952440, rs881598, rs2291955, rs2518054, rs866038, rs1436425, rs173537, rs247611, rs166017, rs173538, rs193694, rs7205692, rs8048746, rs247618, rs183130, rs6499863, rs4783961, rs3816117, rs711752, rs708272, rs1864163, rs4369653, rs1864165, rs891141, rs891143, rs7205804, rs5885, rs1532625, rs1532624, rs289712, rs7499892, rs5883, rs289714, rs158480, rs289717, rs4344729, rs289718, rs289719, rs2033254, rs4784744, rs291044, rs8053613, rs5881, rs5880, rs7198026, rs5882, rs8045701, rs289741, rs1801706, rs289742, rs289743, rs289746, rs172337, rs289747, rs1566439, rs7205459, rs289749, rs289751, rs8059220, rs8058353, rs289735, rs289737, rs291042, rs1875236, rs821466, rs821465, rs4275846, rs289707, rs821463, rs289706, rs1167741, rs2052880, rs1167742, rs1183256, rs1651665, rs1651666, rs4784751, rs1651667, rs8052091, rs1684574, rs1684575, rs1672865, rs821470, rs1549669, rs291040 and rs289754.

19. The method of claim 1, wherein detecting the presence or absence of the one or more polymorphic variations comprises:
hybridizing an oligonucleotide to the nucleic acid sample, wherein the oligonucleotide is complementary to a nucleotide sequence in the nucleic acid and hybridizes to a region adjacent to the polymorphic variation;
extending the oligonucleotide in the presence of one or more nucleotides, yielding extension products; and
detecting the presence or absence of a polymorphic variation in the extension products.

20. The method of claim 1, wherein the subject is a human.

21-55. (canceled)
